(19) United States
(12) Patent Application Publication
(10) Pub. No.: US 2026/0078381 A1
(43) Pub. Date: Mar. 19, 2026

(54) Methods for Directing Lipid Nanoparticles in Vivo

(71) Applicant: Vertex Pharmaceuticals Incorporated, Boston, MA (US)

(72) Inventors: Gregoriy Aleksandrovich Dokshin, Boston, MA (US); Souvik Biswas, Stow, MA (US)

(73) Assignee: Vertex Pharmaceuticals Incorporated, Boston, MA (US)

(21) Appl. No.: 19/400,453

(22) Filed: Nov. 25, 2025

Related U.S. Application Data
(63) Continuation of application No. PCT/US24/31121, filed on May 24, 2024.
(60) Provisional application No. 63/469,156, filed on May 26, 2023.

(51) Int. Cl. C12N 15/113

(52) U.S. Cl. CPC C12N 15/1138; C12N 2310/14; C12N 2310/351; C12N 2310/3515

(57) ABSTRACT

Compositions and methods for enhancing payload-based gene therapy by increasing the percentage of payload delivered to a non-liver target in a subject by blocking binding of LDL to LDL receptors (LDLR) in the liver, and then administering a payload-based therapy targeting a non-liver tissue.

This application is a continuation of International Application No. PCT/US2024/031121, filed May 24, 2024, which claims the benefit of priority to U.S. Provisional Application No. 63/469,156, filed May 26, 2023, each of which is incorporated herein by reference in its entirety.

The application contains a Sequence Listing which has been submitted electronically in .XML format and is hereby incorporated by reference in its entirety. Said .XML copy, created on May 22, 2024, is named “01245-0041-00PCT.xml” and is 15,986,166 bytes in size. The sequence listing contained in this .XML file is part of the specification and is hereby incorporated by reference herein in its entirety.

### INTRODUCTION AND SUMMARY

Genetic modification as a therapeutic is becoming more common as pharmaceutically acceptable compositions such as small interfering RNA (siRNA), messenger RNA (mRNA), antisense oligonucleotides (ASOs), and CRISPR-Cas systems are successfully implemented in human subjects. However, such molecules and systems typically require the use of a delivery system to shield the therapeutic from in vivo degradation. A current problem with the current delivery systems is their inherent tropism for unwanted in vivo targets, such as, for example, the liver.

Adeno-associated viruses (AAV) vectors have been a leading candidate for in vivo gene therapy because of their broad tissue tropism, non-pathogenic nature and low immunogenicity. Currently twelve AAV serotypes and over 100 variants have been identified in human and nonhuman primate populations. Gene therapy vectors using AAV in vivo can infect both dividing and quiescent cells and persist in an extrachromosomal state without integrating into the genome of the host cell. These features make AAV an attractive candidate for use as delivery systems. However, AAV vectors are limited by several factors, including their small packaging size. Moreover, current limitations of using AAVs for gene transfer include potential safety concerns, including possible off-target toxicity. Following administration, in addition to localized and targeted delivery, most serotypes of AAV may also achieve off-target gene transfer, which can result in transduction and expression of the gene of interest in unwanted cells or tissues such as, for example, the liver. When AAV vectors reach the bloodstream, the circulatory system carries the vectors to the whole body, including to the liver, skeletal and cardiac muscles, pancreas and adrenal glands. While different AAV serotypes can have distinct tissue distribution patterns after administration, the liver is the most common organ harboring a large amount of mis-targeted AAV vectors. A recent biodistribution animal study found that, despite direct cerebrospinal fluid administration, biodistribution of vector DNA and green fluorescent protein (GFP) expression was widespread. See Meseck et al., doi.org/10.1101/2021.11.28.470258, BioRxiv.org (posted Nov. 28, 2021). In a portion of that study, the transduction and expression of scAAV9-CB-GFP in the CNS and peripheral tissues following a single intrathecal infusion into the cerebrospinal fluid was assessed. Vector DNA and GFP expression was found to be the greatest in the spinal cord, dorsal root ganglia, and systemic tissue (e.g., liver) with lower concentrations in many brain regions. Recently in 2020-2021, a gene therapy clinical trial using AAV gene therapy was paused due in part to deaths of subjects who developed complications from liver failure. See NCT03199469 (using an AAV serotype 8 vector). Thus, there exists a need in the art to reduce hepatotoxicity from delivery of genetic drugs via AAVs.

Non-viral delivery systems, including lipid nanoparticles (LNPs) and lipoplexes, provide an attractive and promising alternative to AAVs for genetic drug delivery in vivo. Specifically, LNPs are ideal candidates for at least the reason that LNPs provide for efficient biocompatibility, delivery of oligonucleotides into target cells, structural flexibility, rapid elimination after the target function is performed, low toxicity and immunity, and ease of large-scale preparation. However, LNPs are known to have some of the same disadvantages of AAV delivery systems, in particular, in off-target delivery to non-desired tissues, such as, for example, the liver. See, e.g., Dilliard, S A and Siegward, D J, 2022, Nature Biomedical Engineering, 6:106-107.

The present invention, in part, seeks to improve genetic therapies by blocking or reducing the off-target effects of delivery vehicles to the liver. For example, by knocking down or blocking low-density lipoprotein (LDL) binding to a low-density lipoprotein receptor (LDLR) using an agent (for example, an siRNA or anti-sense oligonucleotide), as a pre-conditioning therapy prior to or concurrently with receiving a therapeutic “payload” encapsulated or associated with an LNP, or lipoplex, one can block or reduce off-target liver delivery and toxicity. Because RNAi-based pre-conditioning can be administered prior to the LNP or lipoplex, carrying a therapeutic payload, and can later be cleared from the body, it can effectively and temporarily block LDL binding to LDLR in the liver, thereby enhancing the tropism of a payload to the intended non-liver target, without long term effects. The present compositions and methods can be used as a platform to enhance the tropism and thus efficacy and safety of gene therapies.

Accordingly, the following non-limiting embodiments are provided:
Embodiment 1 is a composition comprising an agent that blocks low-density lipoprotein (LDL) binding to a low-density lipoprotein receptor (LDLR) and a delivery molecule, wherein the delivery molecule is capable of delivering the agent to the liver.
Embodiment 2 is the composition of embodiment 1, further comprising a payload.
Embodiment 3 is the composition of embodiment 2, wherein the payload comprises a therapeutic agent.
Embodiment 4 is the composition of embodiment 2 or 3, wherein the payload comprises a component of a CRISPR/Cas system or a nucleic acid encoding one or more component of a CRISPR/Cas system, a biologic, or a small molecule, optionally wherein the component of a CRISPR/Cas system comprises a nucleic acid encoding one or more guide RNAs, one or more scaffolds, and/or one or more endonucleases.
Embodiment 5 is the composition of any one of embodiments 2-4, further comprising a lipid nanoparticle (LNP) or lipoplex, wherein the LNP or lipoplex encapsulates the payload.
Embodiment 6 is the composition of any one of embodiments 1-5, wherein the delivery molecule comprises a lipid nanoparticle (LNP) or lipoplex.
Embodiment 7 is the composition of any one of embodiments 1-5, wherein the delivery molecule comprises one or more of the following: lactose, galactose, N-acetylgalactosamine (GalNAc), galactosamine, N-formylgalactosamine, N-acetylgalactosamine, N-propionylgalactosamine, N-butanoylgalactosamine, N-isobutanoyl-galactosamine, and cholesterol, or a derivative thereof.
Embodiment 8 is the composition of embodiment 7, wherein the delivery molecule comprises N-acetylgalactosamine (GalNAc).
Embodiment 9 is the composition of any one of embodiments 1-8, wherein the agent comprises an RNAi.
Embodiment 10 is the composition of embodiment 9, wherein the RNAi is a small-interfering RNA (siRNA), an antisense oligonucleotide (ASO), microRNA (miRNA), double-stranded RNA (dsRNA), short hairpin RNA (shRNA) or an expression cassette encoding an RNA.
Embodiment 11 is the composition of embodiment 9, wherein the agent comprises a siRNA.
Embodiment 12 is the composition of embodiment 11, wherein the siRNA binds to a nucleic acid comprising the nucleotide sequence of any one of SEQ ID NOs: 350-352, or the reverse complement thereof.
Embodiment 13 is the composition of embodiment 11, wherein the siRNA comprises at least 13, at least 14, at least 15, at least 16, at least 17, at least 18, at least 19, at least 20, or at least 21 consecutive nucleic acids of any one of the nucleic acid sequences in Table 3A or 3B.
Embodiment 14 is the composition of embodiment 11, wherein the siRNA is at least 80%, at least 85%, at least 90%, or at least 95% identical to any one of the nucleic acid sequences in Table 3A or 3B.
Embodiment 15 is the composition of embodiment 11, wherein the siRNA comprises at least 13, at least 14, at least 15, at least 16, at least 17, at least 18, at least 19, at least 20, or at least 21 consecutive amino acids of an amino acid sequence selected from SEQ ID NOs: 4300-4309.
Embodiment 16 is the composition of embodiment 11, wherein the siRNA is at least 80%, at least 85%, at least 90%, or at least 95% identical to an amino acid sequence selected from SEQ ID NOs: 4300-4309.
Embodiment 17 is the composition of embodiment 12, wherein the agent comprises an antisense oligonucleotide (ASO).
Embodiment 18 is the composition of any one of embodiments 1-17, wherein the LDLR comprises an amino acid sequence that is at least 85%, 90%, 91%, 92%, 93%, 94%, 95%, 96%, 97%, 98%, 99% or 100% identical to any one of the sequences of SEQ ID NOs: 353-355.
Embodiment 19 is the composition of embodiment 4, wherein the component of the CRISPR/Cas system comprises a nucleic acid encoding one or more guide RNAs and a Cas9, wherein the nucleic acid molecule comprises:
   a. a first nucleic acid encoding one or more guide RNAs selected from any one of SEQ ID NOs: 1-35, 1000-1078, or 3000-3069 and a second nucleic acid encoding a Staphylococcus aureus Cas9 (SaCas9); or
   b. a first nucleic acid encoding one or more guide RNAs selected from any one of SEQ ID NOs: 100-225, 2000-2116, or 4000-4251 and a second nucleic acid encoding a Staphylococcus lugdunensis (SluCas9); or
   c. a first nucleic acid encoding one or more guide RNAs comprising at least 20 contiguous nucleotides of a guide RNA selected from any one of SEQ ID NOs: 1-35, 1000-1078, or 3000-3069 and a second nucleic acid encoding a Staphylococcus aureus Cas9 (SaCas9); or
   d. a first nucleic acid encoding one or more guide RNAs comprising at least 20 contiguous nucleotides of a guide RNA selected from any one of SEQ ID NOs: 100-225, 2000-2116, or 4000-4251 and a second nucleic acid encoding a Staphylococcus lugdunensis (SluCas9); or
   e. a first nucleic acid encoding one or more guide RNAs that is at least 90% identical to any one of SEQ ID NOs: 1-35, 1000-1078, or 3000-3069 and a second nucleic acid encoding a Staphylococcus aureus Cas9 (SaCas9); or
   f. a first nucleic acid encoding one or more guide RNAs that is at least 90% identical to any one of SEQ ID NOs: 100-225, 2000-2116, or 4000-4251 and a second nucleic acid encoding a Staphylococcus lugdunensis (SluCas9); or
   g. a first nucleic acid encoding a pair of guide RNAs comprising a first and second guide RNA selected from any one of the following pairs of guide RNAs: SEQ ID NOs: 1020 and 23; 1023 and 23; 1023 and 1037; 1024 and 1055; 1025 and 23; 1025 and 1055; 1026 and 23; 1028 and 1055; 1029 and 1055; 1029 and 1037; 1031 and 1037; 1032 and 1037; 101029 and 1027; 1037 and 1048; 1037 and 1051; 1037 and 1053; 20 and 23; 1038 and 23; 21 and 23; 1040 and 23; 1042 and 1037; 1043 and 1037; 1044 and 1037; 1045 and 1037; 1046 and 1037; 24 and 1037; 1047 and 1055; or 1055 and 1022; and a second nucleic acid encoding a Staphylococcus aureus Cas9 (SaCas9); or
   h. a first nucleic acid encoding a pair of guide RNAs comprising a first and second guide RNA selected from any one of the following pairs of guide RNAs: SEQ ID NOs: 170 and 179; 172 and 179; 179 and 183; 179 and 185; 179 and 187; 179 and 188; 179 and 189; 179 and 193; 179 and 195; 179 and 196; 179 and 197; 200 and 174; or 200 and 176; and a second nucleic acid encoding a Staphylococcus lugdunensis (SluCas9);
   i. a first nucleic acid encoding a pair of guide RNAs comprising a first and second guide RNA selected from any one of the following pairs of guide RNAs: SEQ ID NOs: 117 and 121; 117 and 122; 120 and 121; 120 and 123; 120 and 124; 120 and 125; 122 and 126; 122 and 123; 122 and 124; 122 and 125; or 122 and 126; and a second nucleic acid encoding a Staphylococcus lugdunensis (SluCas9); for targeting exon 44;
   j. a first nucleic acid encoding a pair of guide RNAs comprising a first and second guide RNA selected from any one of the following pairs of guide RNAs: 155 and 156; 155 and 158; 155 and 162; 155 and 163; 162 and 157; 162 and 159; 162 and 164; 162 and 166; or 162 and 167; and a second nucleic acid encoding a Staphylococcus lugdunensis (SluCas9); for targeting exon 50;
   k. a first nucleic acid encoding a pair of guide RNAs comprising a first and second guide RNA selected from any one of the following pairs of guide RNAs: SEQ ID NOs: 211 and 223; 211 and 225; 214 and 224; 216 and 223; 216 and 225; 220 and 224; 204 and 223; 223 and 224; or 204 and 225; and a second nucleic acid encoding a Staphylococcus lugdunensis (SluCas9); for targeting exon 53;
   l. a first nucleic acid encoding a pair of guide RNAs comprising a first and second guide RNA selected from any one of the following pairs of guide RNAs: SEQ ID NOs: 1068 and 32; 1069 and 32; 1070 and 1075; 1071 and 32; 29 and 1075; 1072 and 27; 1072 and 28; 1072 and 32; 1072 and 33; 1073 and 1076; 1073 and 35; 221 and 1077; 1074 and 27; 1074 and 28; 1074 and 33; 32 and 1077; 1075 and 1076; 1075 and 35; 1076 and 26; or 35 and 26; and a second nucleic acid encoding a Staphylococcus aureus Cas9 (SaCas9); for targeting exon 53;
   m. a first nucleic acid encoding a pair of guide RNAs comprising a first and second guide RNA selected from any one of the following pairs of guide RNAs: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
   n. a first nucleic acid encoding a pair of guide RNAs comprising a first and second guide RNA selected from any one of the following pairs of guide RNAs: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9).
Embodiment 20 is a method comprising (a) administering to a subject in need thereof an agent that blocks LDL binding to a low-density lipoprotein receptor (LDLR) and concurrently or sequentially (b) administering an LNP or lipoplex comprising a payload to the subject, wherein the agent reduces off-target delivery of the LNP or lipoplex to the liver.
Embodiment 21 is a method of increasing the percentage of payload delivered to a non-liver target in a subject, comprising (a) a pre-conditioning step comprising administering to the subject a composition comprising an agent that blocks LDL binding to a low-density lipoprotein receptor (LDLR) in the liver, and then (b) administering an LNP or lipoplex and a payload to the subject.
Embodiment 22 is the method of any one of embodiments 20-21, wherein step (a) comprises administering to the subject the composition of any one of embodiments 1, and 6-18.
Embodiment 23 is a method of decreasing liver tropism of a payload administered in an LNP or lipoplex in a subject comprising (a) administering to the subject a composition of any one of embodiments 1, and 6-18, and then (b) administering an LNP or lipoplex and a payload to the subject.
Embodiment 24 is the method of any of embodiments 20-23, wherein administering to the subject a composition comprising an agent that blocks LDL binding to a low-density lipoprotein receptor (LDLR) in the liver temporarily blocks the delivery molecule from interacting with the LDLR in the liver.
Embodiment 25 is the method of any of embodiments 20-24, wherein administering to the subject the composition and/or agent that blocks LDL binding to a low-density lipoprotein receptor (LDLR) in the liver occurs about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days prior to administering the LNP or lipoplex, and payload.
Embodiment 26 is the method of any one of embodiments 20-24, wherein administering to the subject the composition and/or agent that blocks LDL binding to a low-density lipoprotein receptor (LDLR) in the liver occurs about 1 day, about 2 days, about 3 days, about 4 days, about 5 days, about 6 days, about 7 days prior to administering the LNP or lipoplex and payload.
Embodiment 27 is the method of any of embodiments 20-24, wherein administering to the subject the composition and/or agent that blocks LDL binding to a low-density lipoprotein receptor (LDLR) in the liver occurs about 8 days, about 9 days, about 10 days, about 11 days, about 12 days, about 13 days, or about 14 days prior to administering the LNP or lipoplex and payload.
Embodiment 28 is the method of any of embodiments 20-24 wherein administering to the subject the composition of any one of embodiments 1, and 6-18 immediately precedes administering the LNP or lipoplex and payload.
Embodiment 29 is the method of any of embodiments 20-24, wherein the composition of any one of embodiments 1, and 6-18 and the LNP or lipoplex and payload are co-administered.
Embodiment 30 is the method of any of embodiments 20-24, wherein the delivery molecule is a lipid nanoparticle (LNP).
Embodiment 31 is the method of any of embodiments 20-24, wherein the payload is a component of a CRISPR/Cas system or a nucleic acid encoding one or more components thereof, a biologic, or a small molecule, optionally wherein the component of a CRISPR/Cas system comprises a nucleic acid encoding one or more guide RNAs, one or more scaffolds, and/or one or more endonucleases.
Embodiment 32 is the method of embodiment 31, wherein the component of a CRISPR/Cas system comprises a nucleic acid encoding one or more guide RNAs and a Cas9, wherein the nucleic acid molecule comprises:
   a. a first nucleic acid encoding one or more guide RNAs selected from any one of SEQ ID NOs: 1-35, 1000-1078, or 3000-3069 and a second nucleic acid encoding a Staphylococcus aureus Cas9 (SaCas9); or
   b. a first nucleic acid encoding one or more guide RNAs selected from any one of SEQ ID NOs: 100-225, 2000-2116, or 4000-4251 and a second nucleic acid encoding a Staphylococcus lugdunensis (SluCas9); or
   c. a first nucleic acid encoding one or more guide RNAs comprising at least 20 contiguous nucleotides of a guide RNA selected from any one of SEQ ID NOs: 1-35, 1000-1078, or 3000-3069 and a second nucleic acid encoding a Staphylococcus aureus Cas9 (SaCas9); or
   d. a first nucleic acid encoding one or more guide RNAs comprising at least 20 contiguous nucleotides of a guide RNA selected from any one of SEQ ID NOs: 100-225, 2000-2116, or 4000-4251 and a second nucleic acid encoding a Staphylococcus lugdunensis (SluCas9); or
   e. a first nucleic acid encoding one or more guide RNAs that is at least 90% identical to any one of SEQ ID NOs: 1-35, 1000-1078, or 3000-3069 and a second nucleic acid encoding a Staphylococcus aureus Cas9 (SaCas9); or
   f. a first nucleic acid encoding one or more guide RNAs that is at least 90% identical to any one of SEQ ID NOs: 100-225, 2000-2116, or 4000-4251 and a second nucleic acid encoding a Staphylococcus lugdunensis (SluCas9); or
   g. a first nucleic acid encoding a pair of guide RNAs comprising a first and second guide RNA selected from any one of the following pairs of guide RNAs: SEQ ID NOs: 1020 and 23; 1023 and 23; 1023 and 1037; 1024 and 1055; 1025 and 23; 1025 and 1055; 1026 and 23; 1028 and 1055; 1029 and 1055; 1029 and 1037; 1031 and 1037; 1032 and 1037; 101029 and 1027; 1037 and 1048; 1037 and 1051; 1037 and 1053; 20 and 23; 1038 and 23; 21 and 23; 1040 and 23; 1042 and 1037; 1043 and 1037; 1044 and 1037; 1045 and 1037; 1046 and 1037; 24 and 1037; 1047 and 1055; or 1055 and 1022; and a second nucleic acid encoding a Staphylococcus aureus Cas9 (SaCas9); or
   h. a first nucleic acid encoding a pair of guide RNAs comprising a first and second guide RNA selected from any one of the following pairs of guide RNAs: SEQ ID NOs: 170 and 179; 172 and 179; 179 and 183; 179 and 185; 179 and 187; 179 and 188; 179 and 189; 179 and 193; 179 and 195; 179 and 196; 179 and 197; 200 and 174; or 200 and 176; and a second nucleic acid encoding a Staphylococcus lugdunensis (SluCas9);
   i. a first nucleic acid encoding a pair of guide RNAs comprising a first and second guide RNA selected from any one of the following pairs of guide RNAs: SEQ ID NOs: 117 and 121; 117 and 122; 120 and 121; 120 and 123; 120 and 124; 120 and 125; 122 and 126; 122 and 123; 122 and 124; 122 and 125; or 122 and 126; and a second nucleic acid encoding a Staphylococcus lugdunensis (SluCas9); for targeting exon 44;
   j. a first nucleic acid encoding a pair of guide RNAs comprising a first and second guide RNA selected from any one of the following pairs of guide RNAs: 155 and 156; 155 and 158; 155 and 162; 155 and 163; 162 and 157; 162 and 159; 162 and 164; 162 and 166; or 162 and 167; and a second nucleic acid encoding a Staphylococcus lugdunensis (SluCas9); for targeting exon 50;
   k. a first nucleic acid encoding a pair of guide RNAs comprising a first and second guide RNA selected from any one of the following pairs of guide RNAs: SEQ ID NOs: 211 and 223; 211 and 225; 214 and 224; 216 and 223; 216 and 225; 220 and 224; 204 and 223; 223 and 224; or 204 and 225; and a second nucleic acid encoding a Staphylococcus lugdunensis (SluCas9); for targeting exon 53;
   l. a first nucleic acid encoding a pair of guide RNAs comprising a first and second guide RNA selected from any one of the following pairs of guide RNAs: SEQ ID NOs: 1068 and 32; 1069 and 32; 1070 and 1075; 1071 and 32; 29 and 1075; 1072 and 27; 1072 and 28; 1072 and 32; 1072 and 33; 1073 and 1076; 1073 and 35; 221 and 1077; 1074 and 27; 1074 and 28; 1074 and 33; 32 and 1077; 1075 and 1076; 1075 and 35; 1076 and 26; or 35 and 26; and a second nucleic acid encoding a Staphylococcus aureus Cas9 (SaCas9); for targeting exon 53;
   m. a first nucleic acid encoding a pair of guide RNAs comprising a first and second guide RNA selected from any one of the following pairs of guide RNAs: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
   n. a first nucleic acid encoding a pair of guide RNAs comprising a first and second guide RNA selected from any one of the following pairs of guide RNAs: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9).
Embodiment 33 is the method of any one of embodiments 20-32, wherein the payload is encapsulated within a lipid nanoparticle (LNP).
Embodiment 34 is the method of any of embodiments 20-33, wherein the method comprises co-administering other drugs that facilitate increased uptake of the agent in the liver.
Embodiment 35 is the method of any of any of embodiments 20-34 wherein the blocking of LDL to a low-density lipoprotein receptor (LDLR) in the liver in step (a) is not temporary.
Embodiment 36 is the method of any one of embodiments 20-35, wherein the payload is within an LNP, and the LNP targets the brain; spinal cord; eye; retina; bone; cardiac muscle, skeletal muscle, smooth muscle; lung; pancreas; heart; and/or kidney.
Embodiment 37 is the method of any one of embodiments 20-36, wherein administering the composition in step (a) increases the percentage of payload delivered to a non-liver target.
Embodiment 38 is the method of any one of embodiments 20-37, wherein the method results in at least a 10%, 30%, 50%, 75%, 100%, 125%, 150%, 200%, 250%, 300%, 350%, 400%, 450%, 500%, 600%, 700%, 800%, 900%, or 1000% increase of payload in a non-liver target as compared to the payload in the corresponding tissue of a control subject that received the payload but did not receive the agent that blocks LDL binding to a low-density lipoprotein receptor (LDLR) in the liver.
Embodiment 39 is the method of embodiment 38, wherein the method results in at least a 10%, 30%, 50%, 75%, 100%, 125%, 150%, 200%, 250%, 300%, 350%, 400%, 450%, 500%, 600%, 700%, 800%, 900%, or 1000% increase of payload in skeletal muscle as compared to the payload in the corresponding muscle of a control subject that received the payload but did not receive the agent that blocks LDL binding to a low-density lipoprotein receptor (LDLR) in the liver.
Embodiment 40 is the method of any one of embodiments 20-39, wherein the agent comprises an RNAi.
Embodiment 41 is the method of embodiment 40, wherein the RNAi is a small-interfering RNA (siRNA), an antisense oligonucleotide (ASO), microRNA (miRNA), double-stranded RNA (dsRNA), short hairpin RNA (shRNA) or an expression cassette encoding an RNA.
Embodiment 42 is the method of embodiment 41, wherein the agent comprises a siRNA.
Embodiment 43 is the method of embodiment 42, wherein the siRNA binds to a nucleic acid comprising the nucleotide sequence of any one of SEQ ID NOs: 350-352, or the reverse complement thereof.
Embodiment 44 is the method of embodiment 42, wherein the siRNA comprises at least 13, at least 14, at least 15, at least 16, at least 17, at least 18, at least 19, at least 20, or at least 21 consecutive nucleic acids of any one of the nucleic acid sequences in Table 3A or 3B.
Embodiment 45 is the method of embodiment 42, wherein the siRNA is at least 80%, at least 85%, at least 90%, or at least 95% identical to any one of the nucleic acid sequences in Table 3A or 3B.
Embodiment 46 is the method of embodiment 42, wherein the siRNA comprises at least 13, at least 14, at least 15, at least 16, at least 17, at least 18, at least 19, at least 20, or at least 21 consecutive amino acids of an amino acid sequence selected from SEQ ID NOs: 4300-4309.
Embodiment 47 is the method of embodiment 42, wherein the siRNA is at least 80%, at least 85%, at least 90%, or at least 95% identical to an amino acid sequence selected from SEQ ID NOs: 4300-4309.
Embodiment 48 is the method of embodiment 42, wherein the agent comprises an antisense oligonucleotide (ASO).
Embodiment 49 is the method of any of embodiments 20-48, wherein the subject is a human subject.
Embodiment 50 is the method of any one of embodiments 20-49, wherein the agent is administered intravenously or subcutaneously.
Embodiment 51 is a composition comprising a siRNA comprising a nucleotide sequence selected from SEQ ID NOs: 4300-15629.
Embodiment 52 is the composition of embodiment 51, wherein the siRNA comprises a nucleotide sequence selected from any one of SEQ ID NOs: 4300-4309.

### BRIEF DESCRIPTION OF THE DRAWINGS

FIGS. 1A-J show knockdown of LDLR mRNA by 10 different siRNAs in Hepa1-6 cells. The plots show 10-point dose response for each of the 10 siRNAs evaluated. FIG. 1K provides siRNA sequence identity, species conservation and potency values calculated from dose-response curves.

FIG. 2 shows knockdown of LDLR mRNA with different siRNA in Hepa1-6 cells. The plot trace shows fold change in LDLR mRNA levels relative to untreated samples, as measured by qPCR 48 hours after treatment with 2.5 nM (left column of modality), 50 nM siRNA (right column corresponding to treatment with one modality). The dashed black line indicates 50% mRNA expression level. The bar height represents the mean value of the technical replicates and error bars correspond to group standard deviation. Positive control is commercially available siRNA targeting LDLR (Invitrogen AM16708-“Invt”). All siRNa were transfected to cells using standard RNAi Max protocol. Negative control: non-targeting siRNA (51-01-19-08, Integrated DNA Technologies) used as a negative control. Not treated: samples were not treated with lipofectamine.

FIG. 3 shows a simplified graphical representation of the in vivo study design and analysis to examine LDLR protein knockdown kinetics.

FIG. 4 shows in vivo data for the LNP siRNA results in sustainable knockdown of LDLR mRNA through day 7.

FIGS. 5A-B show in vivo data wherein, compared to PBS, siRNA-LNP results in sustainable knockdown of LDLR protein through day 7. FIG. 5A shows representative image of Jess blot for selected samples from Day 1 and LDLR recombinant protein. FIG. 5B shows normalized LDLR protein expression level from mouse liver at each timepoint. Samples were normalized to mean value of PBS treated animals at a given timepoint.

FIG. 6 shows in vivo data, wherein LDL-c (low density lipoprotein cholesterol) acts as biomarker of LDLR knockdown. Animals dosed with LDLR-targeting articles show an increase in LDL-c levels. Each dot point on the graph represents the LDL-c level of a single animal. The bar height represents the mean result of the group, and error bars represent standard deviation of the group. Control PBS contains results from IV and SC ROA. The results from one animal were excluded due to a failed experiment.

FIG. 7 is a simplified graphical representation of an in vivo study design and analysis to examine LDLR-LNP doses, wherein mice will be dosed with a range of liver-tropic LDLR targeting siRNA-LNPs amounts and tissues will be harvested 4 to 48h after dosing to evaluate LDLR protein levels by western blot. The figure corresponds to the study in Example 3.

FIG. 8 is a simplified graphical representation of an in vivo study design and analysis to examine LDLR protein knockdown kinetics from optimal dose corresponding to the study in Example 4.

FIG. 9 is a simplified graphical representation of an in vivo proof of concept study design and analysis to examine the efficiency of LNP de-targeting strategy. The study corresponds to the study in Example 5.

### DETAILED DESCRIPTION

Reference will now be made in detail to certain embodiments of the invention. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a guide” includes a plurality of guides and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless stated otherwise, the following terms and phrases as used herein are intended to have the following meanings:

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA. The disclosure provides a number of exemplary nucleotide sequences herein, and contemplates reverse complements of these nucleotide sequences, as well as RNA and/or DNA equivalents of any of these sequences. For example, an RNA equivalent of any of the DNA sequences disclosed herein would comprise uracils in place of thymines in the sequence, whereas a DNA equivalent of any of the RNA sequences disclosed herein would comprise thymines in place of uracils.

As used herein, “CRISPR” systems and “RNA-targeted endonucleases” or “Cas-nucleases” includes the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. In particular embodiments, the RNA-targeted endonuclease is a type II CRISPR Cas enzyme. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., Nat. Rev. Microbiol., 9:467-477 (2011); Makarova et al., Nat. Rev. Microbiol., 13: 722-36 (2015); Shmakov et al., Molecular Cell, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

“Guide RNA”, “guide RNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “guide RNA” refers to each type. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues.

As used herein, a “spacer sequence,” sometimes also referred to herein and in the literature as a “spacer,” “protospacer,” “guide sequence,” or “targeting sequence” refers to a sequence within a guide RNA that is complementary to a target sequence and functions to direct a guide RNA to a target sequence for cleavage by a Cas9. A guide sequence can be 24, 23, 22, 21, 20 or fewer base pairs in length, e.g., in the case of Staphylococcus lugdunensis (i.e., SluCas9) or Staphylococcus aureus (i.e., SaCas9) and related Cas9 homologs/orthologs. In preferred embodiments, a guide/spacer sequence in the case of SluCas9 or SaCas9 is at least 20 base pairs in length, or more specifically, within 20-25 base pairs in length (see, e.g., Schmidt et al., 2021, Nature Communications, “Improved CRISPR genome editing using small highly active and specific engineered RNA-guided nucleases”). Shorter or longer sequences can also be used as guides, e.g., 15-, 16-, 17-, 18-, 19-, 20-, 21-, 22-, 23-, 24-, or 25-nucleotides in length. For example, in some embodiments, the guide sequence comprises at least 17, 18, 19, 20, 21, 22, 23, 24, or 25 contiguous nucleotides of a sequence selected from SEQ ID NOs: 1-35 (for SaCas9), and 100-225 (for SluCas9). In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides. In some embodiments, the guide sequence and the target region do not contain any mismatches.

As used herein, the terms “about” or “approximately” means an acceptable error for a particular value as determined by one of ordinary skill in the art, which depends in part on how the value is measured or determined.

As used herein, “AAV” refers to an adeno-associated virus vector. As used herein, “AAV” refers to any AAV serotype and variant, including but not limited to an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), AAV9 vector, AAV9P vector also known as AAVMYO (see, Weinmann et al., 2020, Nature Communications, 11:5432), and Myo-AAV vectors described in Tabebordbar et al., 2021, Cell, 184:1-20 (e.g., MyoAAV 1A, 2A, 3A, 4A, 4C, or 4E), wherein the number following AAV indicates the AAV serotype. The term “AAV” can also refer to any known AAV (vector) system. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., Gene Ther. 2001; 8:1248-54, Naso et al., BioDrugs 2017; 31:317-334, and references cited therein for detailed discussion of various AAV vectors. Structurally, AAVs are small (25 nm), single-DNA stranded non-enveloped viruses with an icosahedral capsid. As used herein, “AAV” can refer to naturally occurring or engineered AAV serotypes and recombinant AAVs (rAAVs) and variants that can differ in the composition and structure of their capsid protein have varying tropism, i.e., ability to transduce different cell types. When combined with active promoters, this tropism defines the site of gene expression.

As used herein “payload-based gene therapy” refers to the administration of a payload composition, including one or more Cas9 nucleases and one or more guide RNAs, or nucleic acids encoding one or more Cas9 nucleases and one or more guide RNAs, for gene editing comprising a tissue-specific promoter in facilitating administration of gene therapy, which can include any known gene editing system in the art. A promoter as described herein can also be “cell specific,” meaning that the particular promoter selected for the payload can direct expression of the selected transgene/nucleotide sequence of interest in a particular cell or cell type. In some embodiments, for example, the promoter is a muscle-specific promoter, including a muscle creatine kinase promoter, a desmin promoter, an MHCK7 promoter, or an SPc5-12 promoter. In some embodiments, the muscle-specific promoter is a CK8 promoter. In some embodiments, the muscle-specific promoter is a CK8e promoter. Muscle-specific promoters are described in detail, e.g., in US2004/0175727 A1; Wang et al., Expert Opin Drug Deliv. (2014) 11, 345-364; Wang et al., Gene Therapy (2008) 15, 1489-1499. In some embodiments, the tissue-specific promoter is a neuron-specific promoter, such as an enolase promoter. See, e.g., Naso et al., BioDrugs 2017; 31:317-334; Dashkoff et al., Mol Ther Methods C/in Dev. 2016; 3:16081, and references cited therein for detailed discussion of tissue-specific promoters including neuron-specific promoters. Any known promoters may be used in conjunction with the payloads to administer the gene therapy to the intended target tissues or cells. As used herein, “non-liver payload-based gene therapy” includes treating or preventing a disease or disorder using payload-based gene therapy that is not a disease or disorder of the liver.

As used herein, “low density lipoprotein receptor,” or “LDLR” or “LDL-R” or “LDL receptor” are used interchangeably to refer to LDL receptor protein. LDLR has a Uniprot Accession No. of H0YMD1. Synonyms for LDLR also include, FHC, LDLCQ2, and LDLR_HUMAN. LDLR is a protein that studies have shown is synthesized in the endoplasmic reticulum (ER), where it folds and is partially glycosylated. Next, studies have shown that LDLR is further glycosylated in the Golgi apparatus, rendering the mature protein. The LDLR is organized in five functionally distinct domains: the N-terminal ligand-binding domain, the epidermal growth factor (EGF)-precursor homology domain, the O-linked sugars containing domain, the trans-membrane and the C-terminal cytosolic domain.

As used herein, “blocks LDL binding to an LDLR” or the like, means temporarily or permanently reducing, inhibiting, or blocking the ability of an LDL molecule to bind to an LDL receptor. In some embodiments, “blocking” means downregulating gene expression of an LDLR such that less LDLR is expressed on a cell treated with a “blocking” agent (e.g., LDLR-specific siRNA) as compared to a control cell of the same cell type that is not treated with the “blocking” agent. In some embodiments, “blocking” means contacting the LDLR with an agent (e.g., an antibody or small molecule) that prohibits the binding of an LDL to the LDLR. In particular embodiments, an agent that blocks LDL binding to an LDLR also prevents LNP uptake into liver cells or liver tissue.

As used herein, “preventing LNP uptake into liver cells or tissue” or the like, means temporarily or permanently reducing, inhibiting, or blocking the ability of an LNP (e.g., an LNP carrying a payload) from being taken up by a liver cell or liver tissue by means of the LDL receptor. In particular embodiments, prevention of LNP uptake into liver cells or tissue is accomplished using an agent that blocks LDL binding to an LDLR. In some embodiments, preventing LNP uptake into liver cells or tissue is accomplished by using an agent (e.g., LDLR-specific siRNA or ASO) that downregulates expression of LDLR such that less LDLR is expressed on a cell treated with the agent than as compared to a control cell of the same cell type that is not treated with the agent.

As used herein, “RNAi compound,” “RNAi molecule,” or “RNAi” are used interchangeably and refer to inhibitory RNA. RNAi refers to an antisense compound that acts to modulate a target nucleic acid and/or protein encoded by a target nucleic acid. RNAi compounds include but are not limited to small interfering RNA (siRNA), single-stranded RNA (ssRNA), microRNA, including microRNA mimics, double-stranded RNA (dsRNA), short hairpin RNA (shRNA), and expression cassettes encoding RNA capable of inducing RNA interference. For example, RNAi expression cassettes can be transcribed in cells to produce siRNA, separate sense and anti-sense strand linear siRNA, or small hairpin RNAs that can function as miRNAs. As used herein, referencing “RNAi” and “siRNA” refers to the terms as used in the broadest sense and encompasses, for example, any siRNA that has been modified (e.g., chemical modification, attachment of at least one receptor-binding ligand or moiety) so long as the molecule retains the ability to bind to target nucleic acids in target cells, thereby reducing the target gene's expression. RNAi molecules are readily designed and generated by techniques known in the art.

As used herein, “antisense oligonucleotides” or “ASOs” refer to short strands of DNA or RNA that bind to a complementary RNA sequence, thereby inhibiting its function. ASOs can effectively downregulate or upregulate the production of certain downstream proteins by inhibiting specific RNA sequences, and can theoretically be used with both select loss of function and gain of function mutations. In the context of LDLR, an ASO may be used to downregulate expression of LDLR.

As used herein, “delivery molecule” or “liver targeting moiety” includes, but is not limited to, any molecule, ligand or conjugate that can be applied to an agent that blocks LDL binding to LDLR (e.g., siRNA, RNAi, ASO, or an anti-LDLR antibody) to enhance the agent's delivery and/or uptake by the liver, including any known liver-targeting conjugates, including, N-acetylgalactosamine (GalNAc) conjugates, and any other delivery system for liver or hepatic delivery of the agent (e.g., siRNA or RNAi). The selection of an appropriate molecule, ligand or conjugate for targeting siRNAs to particular body systems, organs, tissues or cells is considered to be within the ordinary skill of the art. For example, to target an siRNA to hepatocytes, cholesterol may be attached at one or more ends, including any combination of 5′- and 3′-ends, of an siRNA molecule. The resultant cholesterol-siRNA is delivered to hepatocytes in the liver, thereby providing a means to deliver siRNAs to this targeted location. Other ligands useful for targeting siRNAs to the liver include HBV surface antigen and low-density lipoprotein (LDL).

As used herein, “LNP” or “lipid nanoparticle” refers to a lipid-based delivery composition. LNPs are known in the art and refer to particles that comprises a plurality of (i.e., more than one) lipid molecules physically associated with each other by intermolecular forces. The LNPs may be, e.g., microspheres (including unilamellar and multilamellar vesicles, e.g., “liposomes”—lamellar phase lipid bilayers that, in some embodiments, are substantially spherical—and, in more particular embodiments, can comprise an aqueous core, e.g., comprising a substantial portion of RNA molecules), a dispersed phase in an emulsion, micelles, or an internal phase in a suspension. Emulsions, micelles, and suspensions may be suitable compositions for local and/or topical delivery. See also, e.g., WO2017173054A1, the contents of which are hereby incorporated by reference in their entirety. Any LNP known to those of skill in the art to be capable of delivering nucleotides, including siRNA or other RNAi, to subjects can be used herein.

As used herein, “lipoplex” (also used interchangeable with “lipid complex” or “lipid:vector complex”) refers to a heterogeneous complex, which self-assembles when nucleic acids (DNA, RNA) are mixed with cationic lipids. The lipid component of the lipoplex can facilitate the delivery of the nucleic acid to cells, as well as protect it from degradation in an extracellular environment. Non-limiting examples for use of a lipoplex include gene therapy and vaccine development.

As used herein, “antibody” is used in the broadest sense and encompasses various antibody structures, including but not limited to monoclonal antibodies, polyclonal antibodies, multispecific antibodies (e.g., bispecific antibodies), nanobodies, and antibody or antigen-binding fragments so long as they exhibit the desired antigen-binding activity. As used herein, “anti-LDLR antibody” refers to an antibody (as used in the broadest sense as set forth above) that blocks an interaction between LDL and LDLR.

The terms “composition” or “formulation” refer to a preparation which is in such form as to permit the biological activity of an active ingredient contained therein to be effective, and which contains no additional components which are unacceptably toxic to a subject to which the composition would be administered.

The terms “pharmaceutically acceptable carrier” or “pharmaceutically acceptable vehicle” refer to any diluent, adjuvant, excipient, or combinations thereof, in a pharmaceutical composition which allows, for example, facilitation of the administration of the active ingredient contained therein. Non-limiting examples of substances that can generally serve as pharmaceutically acceptable carriers include oils, glycols; polyols, such as glycerin, sorbitol, mannitol, and polyethylene glycol; esters; agar; buffering agents; water; isotonic saline solution; Ringer's solution; ethyl alcohol; pH buffer solution; and any other non-toxic compatible materials used in pharmaceutical preparations. Such carriers or vehicles should be non-toxic and should not interfere with the efficacy of the active ingredient. Pharmaceutically acceptable carriers are well known and will be adapted by the person skilled in the art as a function of the nature, route, and mode of administration.

As used herein, “treatment” (and variations thereof such as “treat” or “treating”) refers to any administration or application of a therapeutic for disease or disorder in a subject, and includes inhibiting the disease or development of the disease (which may occur before or after the disease is formally diagnosed, e.g., in cases where a subject has a genotype that has the potential or is likely to result in development of the disease), arresting its development, relieving one or more symptoms of the disease, curing the disease, or preventing reoccurrence of one or more symptoms of the disease. As used herein, “treatment” can include administrating a therapeutic or therapeutic regimen including optional adjuvant or pre-conditioning regimen to achieve a therapeutic or prophylactic benefit. As used herein, “treatment” also encompasses “ameliorating,” which refers to any beneficial effect on a phenotype or symptom, such as reducing its severity, slowing or delaying its development, arresting its development, or partially or completely reversing or eliminating it.

“Pre-conditioning,” “preconditioning,” or “conditioning” are used interchangeably herein and refer to the preparation of the subject in need of the non-liver payload-based gene therapy for a suitable condition, which includes blocking LDL binding to LDL receptors in the liver prior to the subject receiving the payload-based gene therapy.

As used herein, the term “administering” refers to the physical introduction of an agent to a subject, using any of the various methods and delivery systems known to those skilled in the art. Exemplary routes of administration for the agents disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the agents disclosed herein may be administered via a non-parenteral route, e.g., orally. Other non-parenteral routes include a topical, epidermal, or mucosal route of administration, for example, intranasally, vaginally, rectally, sublingually or topically. The phrase “systemic injection” as used herein non-exclusively relates to intravenous, intraperitoneally, subcutaneous, via nasal submucosa, lingual, via bronchoscopy, intravenous, intra-arterial, intra-muscular, intro-ocular, intra-striatal, subcutaneous, intradermal, by dermal patch, by skin patch, by patch, into the cerebrospinal fluid, into the portal vein, into the brain, into the lymphatic system, intra-pleural, retro-orbital, intra-dermal, into the spleen, intra-lymphatic, among others. “Co-administration,” as used herein, means that a plurality of substances are administered sufficiently close together in time so that the agents act together. Co-administration encompasses administering substances together in a single formulation and administering substances in separate formulations close enough in time so that the agents act together.

As used herein, “subject” may be a mammal, such as a primate, ungulate (e.g., cow, pig, horse), cat, dog, domestic pet or domesticated mammal. In some cases, the mammal may be a rabbit, pig, horse, sheep, cow, cat or dog, or a human. In some embodiments, the subject is a human. In some embodiments, the subject is an adult human. In some embodiments, the subject is a juvenile human. In some embodiments, the subject is greater than about 18 years old, greater than about 25 years old, or greater than about 35 years old. In some embodiments, the subject is less than about 18 years old, less than about 16 years old, less than about 14 years old, less than about 12 years old, less than about 10 years old, less than about 8 years old, less than about 6 years old, less than about 5 years old, less than about 4 years old, less than about 3 years old, less than about 2 years old, less than about 1 year old, or less than about 6 months old.

### II. Compositions

Disclosed herein are compositions comprising one or more agents to block and/or that are useful for blocking LDL binding to LDLR, and/or for preventing LNP uptake into liver cells. In some embodiments, the composition comprises an agent that blocks LDL binding to LDLR and a delivery molecule that delivers the agent to the liver. In some embodiments, the composition comprises a) an agent that blocks LDL binding to LDLR and/or for preventing LNP uptake into liver cells or tissue and b) a delivery molecule that delivers the agent to the liver. In some embodiments, the composition further comprises a payload comprising a therapeutic that is optionally encapsulated within or associated with an LNP or lipoplex.

In some embodiments, the composition is capable of temporarily blocking LDL binding to LDLR in the liver and/or for preventing LNP uptake into liver cells or tissue, including for about 48 hours to about 3 weeks. In some embodiments, the composition is capable of permanently blocking LDL binding to LDLR in the liver and/or for preventing LNP uptake into liver cells or tissue. In some embodiments, the composition is capable of blocking LDL binding to LDLR in the liver and/or for preventing LNP uptake into liver cells or tissue for about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days. In some embodiments, the composition is capable of blocking LDL binding to LDLR in the liver and/or for preventing LNP uptake into liver cells or tissue for about 7 days, about 8 days, about 9 days, about 10 days, about 11 days, about 12 days, about 13 days, or about 14 days. In some embodiments, the composition is capable of blocking LDL binding to LDLR in the liver and/or for preventing LNP uptake into liver cells or tissue for about 1-28 days, 7-28 days, 14-28 days, 21-28 days, 1-21 days, 7-21 days, 14-21 days, 1-14 days, 7-14 days, or about 1-7 days.

In some embodiments, a payload and an LNP or lipoplex is administered during or after the administration of the composition. In some embodiments, the composition is capable of long-term blocking of LDL binding to LDLR in the liver and/or for preventing LNP uptake into liver cells or tissue, including for longer than about 3 weeks, longer than about 4 weeks, longer than about 5 weeks, longer than about 6 weeks, longer than about 7 weeks, longer than about 8 weeks, longer than about 9 weeks, and longer than about 10 weeks. In some embodiments, the composition is capable of long-term blocking of LDL binding to LDLR in the liver and/or for preventing LNP uptake into liver cells or tissue, including for longer than 10 weeks. In some embodiments, the composition is capable of blocking LDL binding to LDLR in the liver and/or for preventing LNP uptake into liver cells or tissue for about 10-15 weeks, about 15-20 weeks, about 20-25 weeks, about 25-30 weeks, about 30-35 weeks, about 35-40 weeks, about 40-45 weeks, about 45-50 weeks, or about 50-55 weeks.

### A. RNAi as Agent

In some embodiments, RNA interference (RNAi) is the mechanism by which the agent uses to block LDL binding to LDLR in the liver and/or for preventing LNP uptake into liver cells or tissue. RNA interference refers to sequence- or gene-specific suppression of gene expression (protein synthesis) mediated by RNAi/siRNA in an organism without generally suppressing other protein synthesis. RNAi induces RNA interference through interaction with the RNA interference pathway in mammalian cells in order to degrade or inhibit the translation of messenger RNA (mRNA) transcripts of transgenes in a sequence-specific manner. RNAi directed toward major receptor proteins can lead to decreased protein expression of the target of the RNAi (e.g., LDLR). RNAi can be accomplished through the use of small interfering RNA (siRNA), microRNA (miRNA), double-stranded RNA (dsRNA), short hairpin RNA (shRNA), and expression cassettes encoding RNA capable of inducing RNA interference.

In some embodiments, the agent that blocks LDL binding to LDLR and/or for preventing LNP uptake into liver cells or tissue is small-interfering RNA (siRNA). Small interfering RNAs (siRNAs) are known for their ability to specifically interfere with protein expression in a target. siRNAs are designed to interact with a target ribonucleotide sequence, meaning they complement a target sequence sufficiently to bind to the target sequence. siRNAs generally contain 15-50 base pairs, preferably 21-25 base pairs, and encode a sequence of a target gene or RNA. The siRNA can have complete or partial identity (e.g., complementarity) with its target.

siRNA has been criticized for its transient nature, due to its instability in vivo. However, in this context, siRNA's temporal nature can be a benefit so that the agent is able to block LDL from binding to LDLR and/or for preventing LNP uptake into liver cells or tissue only for a short time during which a payload and LNP or lipoplex are delivered, thereby reducing off-target delivery of the payload to the liver.

In some embodiments, any of the agents, e.g., RNAi molecules and/or compositions disclosed herein, are capable of temporarily blocking LDL binding to LDL receptors (LDLR) and/or for preventing LNP uptake into liver cells or tissue, including for about 48 hours to about 3 weeks. In some embodiments, the agent, e.g., RNAi molecule and/or composition disclosed herein is capable of blocking LDL binding to LDLR and/or for preventing LNP uptake into liver cells or tissue for about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days. In some embodiments, the agent, e.g., RNAi molecule and/or composition disclosed herein is capable of blocking LDL binding to LDLR and/or for preventing LNP uptake into liver cells or tissue for about 7 days, about 8 days, about 9 days, about 10 days, about 11 days, about 12 days, about 13 days, about 14 days. In some embodiments, the agent, e.g., RNAi molecule and/or composition disclosed herein is capable of blocking LDL binding to LDLR and/or for preventing LNP uptake into liver cells or tissue for 1-28 days, 7-28 days, 14-28 days, 21-28 days, 1-21 days, 7-21 days, 14-21 days, 1-14 days, 7-14 days, or 1-7 days.

In some embodiments, the agent, e.g., RNAi molecule and/or composition disclosed herein targets an LDLR-encoding transcript to reduce or block its expression. In some embodiments, the LDLR-encoding transcript comprises a sequence that is at least 80%, 85%, 90%, 91%, 92%, 93%, 94%, 95%, 96%, 97%, 98%, 99% or 100% identical to the sequence of SEQ ID NO: 350, or a reverse complement thereof. In some embodiments, the agent, e.g., RNAi molecule and/or composition disclosed herein targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 350, or a reverse complement thereof. In some embodiments, the agent, e.g., RNAi molecule and/or composition disclosed herein targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 350, or a reverse complement thereof. SEQ ID NO: 350:

GTGCAATCGCGGGAAGCCAGGGTTTCCAGCTAGGACACAGCAGGTCGTGATCCGGGTCG
GGACACTGCCTGGCAGAGGCTGCGAGCATGGGGCCCTGGGGCTGGAAATTGCGCTGGAC
CGTCGCCTTGCTCCTCGCCGCGGCGGGGACTGCAGTGGGCGACAGATGCGAAAGAAACG
AGTTCCAGTGCCAAGACGGGAAATGCATCTCCTACAAGTGGGTCTGCGATGGCAGCGCT
GAGTGCCAGGATGGCTCTGATGAGTCCCAGGAGACGTGCTTGTCTGTCACCTGCAAATCC
GGGGACTTCAGCTGTGGGGGCCGTGTCAACCGCTGCATTCCTCAGTTCTGGAGGTGCGAT
GGCCAAGTGGACTGCGACAACGGCTCAGACGAGCAAGGCTGTCCCCCCAAGACGTGCTC
CCAGGACGAGTTTCGCTGCCACGATGGGAAGTGCATCTCTCGGCAGTTCGTCTGTGACTC
AGACCGGGACTGCTTGGACGGCTCAGACGAGGCCTCCTGCCCGGTGCTCACCTGTGGTCC
CGCCAGCTTCCAGTGCAACAGCTCCACCTGCATCCCCCAGCTGTGGGCCTGCGACAACGA
CCCCGACTGCGAAGATGGCTCGGATGAGTGGCCGCAGCGCTGTAGGGGTCTTTACGTGTT
CCAAGGGGACAGTAGCCCCTGCTCGGCCTTCGAGTTCCACTGCCTAAGTGGCGAGTGCAT
CCACTCCAGCTGGCGCTGTGATGGTGGCCCCGACTGCAAGGACAAATCTGACGAGGAAA
ACTGCGCTGTGGCCACCTGTCGCCCTGACGAATTCCAGTGCTCTGATGGAAACTGCATCC
ATGGCAGCCGGCAGTGTGACCGGGAATATGACTGCAAGGACATGAGCGATGAAGTTGGC
TGCGTTAATGTGACACTCTGCGAGGGACCCAACAAGTTCAAGTGTCACAGCGGCGAATG
CATCACCCTGGACAAAGTCTGCAACATGGCTAGAGACTGCCGGGACTGGTCAGATGAAC
CCATCAAAGAGTGCGGGACCAACGAATGCTTGGACAACAACGGCGGCTGTTCCCACGTC
TGCAATGACCTTAAGATCGGCTACGAGTGCCTGTGCCCCGACGGCTTCCAGCTGGTGGCC
CAGCGAAGATGCGAAGATATCGATGAGTGTCAGGATCCCGACACCTGCAGCCAGCTCTG
CGTGAACCTGGAGGGTGGCTACAAGTGCCAGTGTGAGGAAGGCTTCCAGCTGGACCCCC
ACACGAAGGCCTGCAAGGCTGTGGGCTCCATCGCCTACCTCTTCTTCACCAACCGGCACG
AGGTCAGGAAGATGACGCTGGACCGGAGCGAGTACACCAGCCTCATCCCCAACCTGAGG
AACGTGGTCGCTCTGGACACGGAGGTGGCCAGCAATAGAATCTACTGGTCTGACCTGTCC
CAGAGAATGATCTGCAGCACCCAGCTTGACAGAGCCCACGGCGTCTCTTCCTATGACACC
GTCATCAGCAGAGACATCCAGGCCCCCGACGGGCTGGCTGTGGACTGGATCCACAGCAA
CATCTACTGGACCGACTCTGTCCTGGGCACTGTCTCTGTTGCGGATACCAAGGGCGTGAA
GAGGAAAACGTTATTCAGGGAGAACGGCTCCAAGCCAAGGGCCATCGTGGTGGATCCTG
TTCATGGCTTCATGTACTGGACTGACTGGGGAACTCCCGCCAAGATCAAGAAAGGGGGC
CTGAATGGTGTGGACATCTACTCGCTGGTGACTGAAAACATTCAGTGGCCCAATGGCATC
ACCCTAGATCTCCTCAGTGGCCGCCTCTACTGGGTTGACTCCAAACTTCACTCCATCTCAA
GCATCGATGTCAACGGGGGCAACCGGAAGACCATCTTGGAGGATGAAAAGAGGCTGGCC
CACCCCTTCTCCTTGGCCGTCTTTGAGGACAAAGTATTTTGGACAGATATCATCAACGAA
GCCATTTTCAGTGCCAACCGCCTCACAGGTTCCGATGTCAACTTGTTGGCTGAAAACCTA
CTGTCCCCAGAGGATATGGTTCTCTTCCACAACCTCACCCAGCCAAGAGGAGTGAACTGG
TGTGAGAGGACCACCCTGAGCAATGGCGGCTGCCAGTATCTGTGCCTCCCTGCCCCGCAG
ATCAACCCCCACTCGCCCAAGTTTACCTGCGCCTGCCCGGACGGCATGCTGCTGGCCAGG
GACATGAGGAGCTGCCTCACAGAGGCTGAGGCTGCAGTGGCCACCCAGGAGACATCCAC
CGTCAGGCTAAAGGTCAGCTCCACAGCCGTAAGGACACAGCACACAACCACCCGACCTG
TTCCCGACACCTCCCGGCTGCCTGGGGCCACCCCTGGGCTCACCACGGTGGAGATAGTGA
CAATGTCTCACCAAGCTCTGGGCGACGTTGCTGGCAGAGGAAATGAGAAGAAGCCCAGT
AGCGTGAGGGCTCTGTCCATTGTCCTCCCCATCGTGCTCCTCGTCTTCCTTTGCCTGGGGG
TCTTCCTTCTATGGAAGAACTGGCGGCTTAAGAACATCAACAGCATCAACTTTGACAACC
CCGTCTATCAGAAGACCACAGAGGATGAGGTCCACATTTGCCACAACCAGGACGGCTAC
AGCTACCCCTCGAGACAGATGGTCAGTCTGGAGGATGACGTGGCGTGAACATCTGCCTG
GAGTCCCGTCCCTGCCCAGAACCCTTCCTGAGACCTCGCCGGCCTTGTTTTATTCAAAGA
CAGAGAAGACCAAAGCATTGCCTGCCAGAGCTTTGTTTTATATATTTATTCATCTGGGAG
GCAGAACAGGCTTCGGACAGTGCCCATGCAATGGCTTGGGTTGGGATTTTGGTTTCTTCC
TTTCCTCGTGAAGGATAAGAGAAACAGGCCCGGGGGGACCAGGATGACACCTCCATTTC
TCTCCAGGAAGTTTTGAGTTTCTCTCCACCGTGACACAATCCTCAAACATGGAAGATGAA
AGGGGAGGGGATGTCAGGCCCAGAGAAGCAAGTGGCTTTCAACACACAACAGCAGATG
GCACCAACGGGACCCCCTGGCCCTGCCTCATCCACCAATCTCTAAGCCAAACCCCTAAAC
TCAGGAGTCAACGTGTTTACCTCTTCTATGCAAGCCTTGCTAGACAGCCAGGTTAGCCTT
TGCCCTGTCACCCCCGAATCATGACCCACCCAGTGTCTTTCGAGGTGGGTTTGTACCTTCC
TTAAGCCAGGAAAGGGATTCATGGCGTCGGAAATGATCTGGCTGAATCCGTGGTGGCAC
CGAGACCAAACTCATTCACCAAATGATGCCACTTCCCAGAGGCAGAGCCTGAGTCACTG
GTCACCCTTAATATTTATTAAGTGCCTGAGACACCCGGTTACCTTGGCCGTGAGGACACG
TGGCCTGCACCCAGGTGTGGCTGTCAGGACACCAGCCTGGTGCCCATCCTCCCGACCCCT
ACCCACTTCCATTCCCGTGGTCTCCTTGCACTTTCTCAGTTCAGAGTTGTACACTGTGTAC
ATTTGGCATTTGTGTTATTATTTTGCACTGTTTTCTGTCGTGTGTGTTGGGATGGGATCCC
AGGCCAGGGAAAGCCCGTGTCAATGAATGCCGGGGACAGAGAGGGGCAGGTTGACCGG
GACTTCAAAGCCGTGATCGTGAATATCGAGAACTGCCATTGTCGTCTTTATGTCCGCCCA
CCTAGTGCTTCCACTTCTATGCAAATGCCTCCAAGCCATTCACTTCCCCAATCTTGTCGTT
GATGGGTATGTGTTTAAAACATGCACGGTGAGGCCGGGCGCAGTGGCTCACGCCTGTAA
TCCCAGCACTTTGGGAGGCCGAGGCGGGTGGATCATGAGGTCAGGAGATCGAGACCATC
CTGGCTAACACGTGAAACCCCGTCTCTACTAAAAATACAAAAAATTAGCCGGGCGTGGT
GGCGGGCACCTGTAGTCCCAGCTACTCGGGAGGCTGAGGCAGGAGAATGGTGTGAACCC
GGGAAGCGGAGCTTGCAGTGAGCCGAGATTGCGCCACTGCAGTCCGCAGTCTGGCCTGG
GCGACAGAGCGAGACTCCGTCTCAAAAAAAAAAAACAAAAAAAAACCATGCATGGTGC
ATCAGCAGCCCATGGCCTCTGGCCAGGCATGGCGAGGCTGAGGTGGGAGGATGGTTTGA
GCTCAGGCATTTGAGGCTGTCGTGAGCTATGATTATGCCACTGCTTTCCAGCCTGGGCAA
CATAGTAAGACCCCATCTCTTAAAAAATGAATTTGGCCAGACACAGGTGCCTCACGCCTG
TAATCCCAGCACTTTGGGAGGCTGAGCTGGATCACTTGAGTTCAGGAGTTGGAGACCAG
GCCTGAGCAACAAAGCGAGATCCCATCTCTACAAAAACCAAAAAGTTAAAAATCAGCTG
GGTACGGTGGCACGTGCCTGTGATCCCAGCTACTTGGGAGGCTGAGGCAGGAGGATCGC
CTGAGCCCAGGAGGTGGAGGTTGCAGTGAGCCATGATCGAGCCACTGCACTCCAGCCTG
GGCAACAGATGAAGACCCTATTTCAGAAATACAACTATAAAAAAATAAATAAATCCTCC
AGTCTGGATCGTTTGACGGGACTTCAGGTTCTTTCTGAAATCGCCGTGTTACTGTTGCACT
GATGTCCGGAGAGACAGTGACAGCCTCCGTCAGACTCCCGCGTGAAGATGTCACAAGGG
ATTGGCAATTGTCCCCAGGGACAAAACACTGTGTCCCCCCCAGTGCAGGGAACCGTGAT
AAGCCTTTCTGGTTTCGGAGCACGTAAATGCGTCCCTGTACAGATAGTGGGGATTTTTTG
TTATGTTTGCACTTTGTATATTGGTTGAAACTGTTATCACTTATATATATATATATACACA
CATATATATAAAATCTATTTATTTTTGCAAACCCTGGTTGCTGTATTTGTTCAGTGACTAT
TCTCGGGGCCCTGTGTAGGGGGTTATTGCCTCTGAAATGCCTCTTCTTTATGTACAAAGAT
TATTTGCACGAACTGGACTGTGTGCAACGCTTTTTGGGAGAATGATGTCCCCGTTGTATG
TATGAGTGGCTTCTGGGAGATGGGTGTCACTTTTTAAACCACTGTATAGAAGGTTTTTGT
AGCCTGAATGTCTTACTGTGATCAATTAAATTTCTTAAATGAACCAA.

In some embodiments, the agent, e.g., RNAi molecule and/or composition disclosed herein targets an LDLR-encoding transcript. In some embodiments, the LDLR-encoding transcript comprises a sequence that is at least 80%, 85%, 90%, 91%, 92%, 93%, 94%, 95%, 96%, 97%, 98%, 99% or 100% identical to the sequence of SEQ ID NO: 351, or a reverse complement thereof. In some embodiments, the agent, e.g., RNAi molecule and/or composition disclosed herein targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 351, or a reverse complement thereof. In some embodiments, the agent, e.g., RNAi molecule and/or composition disclosed herein targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 351, or a reverse complement thereof SEQ ID NO: 351:

GTGCAATCGCGGGAAGCCAGGGTTTCCAGCTAGGACACAGCAGGTCGTGATCCGGGTCG
GGACACTGCCTGGCAGAGGCTGCGAGCATGGGGCCCTGGGGCTGGAAATTGCGCTGGAC
CGTCGCCTTGCTCCTCGCCGCGGCGGGGACTGCAGTGGGCGACAGATGCGAAAGAAACG
AGTTCCAGTGCCAAGACGGGAAATGCATCTCCTACAAGTGGGTCTGCGATGGCAGCGCT
GAGTGCCAGGATGGCTCTGATGAGTCCCAGGAGACGTGCTTGTCTGTCACCTGCAAATCC
GGGGACTTCAGCTGTGGGGGCCGTGTCAACCGCTGCATTCCTCAGTTCTGGAGGTGCGAT
GGCCAAGTGGACTGCGACAACGGCTCAGACGAGCAAGGCTGTCCCCCCAAGACGTGCTC
CCAGGACGAGTTTCGCTGCCACGATGGGAAGTGCATCTCTCGGCAGTTCGTCTGTGACTC
AGACCGGGACTGCTTGGACGGCTCAGACGAGGCCTCCTGCCCGGTGCTCACCTGTGGTCC
CGCCAGCTTCCAGTGCAACAGCTCCACCTGCATCCCCCAGCTGTGGGCCTGCGACAACGA
CCCCGACTGCGAAGATGGCTCGGATGAGTGGCCGCAGCGCTGTAGGGGTCTTTACGTGTT
CCAAGGGGACAGTAGCCCCTGCTCGGCCTTCGAGTTCCACTGCCTAAGTGGCGAGTGCAT
CCACTCCAGCTGGCGCTGTGATGGTGGCCCCGACTGCAAGGACAAATCTGACGAGGAAA
ACTGCGCTGTGGCCACCTGTCGCCCTGACGAATTCCAGTGCTCTGATGGAAACTGCATCC
ATGGCAGCCGGCAGTGTGACCGGGAATATGACTGCAAGGACATGAGCGATGAAGTTGGC
TGCGTTAATGTGACACTCTGCGAGGGACCCAACAAGTTCAAGTGTCACAGCGGCGAATG
CATCACCCTGGACAAAGTCTGCAACATGGCTAGAGACTGCCGGGACTGGTCAGATGAAC
CCATCAAAGAGTGCGGGACCAACGAATGCTTGGACAACAACGGCGGCTGTTCCCACGTC
TGCAATGACCTTAAGATCGGCTACGAGTGCCTGTGCCCCGACGGCTTCCAGCTGGTGGCC
CAGCGAAGATGCGAAGATATCGATGAGTGTCAGGATCCCGACACCTGCAGCCAGCTCTG
CGTGAACCTGGAGGGTGGCTACAAGTGCCAGTGTGAGGAAGGCTTCCAGCTGGACCCCC
ACACGAAGGCCTGCAAGGCTGTGGGCTCCATCGCCTACCTCTTCTTCACCAACCGGCACG
AGGTCAGGAAGATGACGCTGGACCGGAGCGAGTACACCAGCCTCATCCCCAACCTGAGG
AACGTGGTCGCTCTGGACACGGAGGTGGCCAGCAATAGAATCTACTGGTCTGACCTGTCC
CAGAGAATGATCTGCAGCACCCAGCTTGACAGAGCCCACGGCGTCTCTTCCTATGACACC
GTCATCAGCAGAGACATCCAGGCCCCCGACGGGCTGGCTGTGGACTGGATCCACAGCAA
CATCTACTGGACCGACTCTGTCCTGGGCACTGTCTCTGTTGCGGATACCAAGGGCGTGAA
GAGGAAAACGTTATTCAGGGAGAACGGCTCCAAGCCAAGGGCCATCGTGGTGGATCCTG
TTCATGGCTTCATGTACTGGACTGACTGGGGAACTCCCGCCAAGATCAAGAAAGGGGGC
CTGAATGGTGTGGACATCTACTCGCTGGTGACTGAAAACATTCAGTGGCCCAATGGCATC
ACCCTAGATCTCCTCAGTGGCCGCCTCTACTGGGTTGACTCCAAACTTCACTCCATCTCAA
GCATCGATGTCAACGGGGGCAACCGGAAGACCATCTTGGAGGATGAAAAGAGGCTGGCC
CACCCCTTCTCCTTGGCCGTCTTTGAGGACAAAGTATTTTGGACAGATATCATCAACGAA
GCCATTTTCAGTGCCAACCGCCTCACAGGTTCCGATGTCAACTTGTTGGCTGAAAACCTA
CTGTCCCCAGAGGATATGGTTCTCTTCCACAACCTCACCCAGCCAAGAGGAGTGAACTGG
TGTGAGAGGACCACCCTGAGCAATGGCGGCTGCCAGTATCTGTGCCTCCCTGCCCCGCAG
ATCAACCCCCACTCGCCCAAGTTTACCTGCGCCTGCCCGGACGGCATGCTGCTGGCCAGG
GACATGAGGAGCTGCCTCACAGAGGCTGAGGCTGCAGTGGCCACCCAGGAGACATCCAC
CGTCAGGCTAAAGGTCAGCTCCACAGCCGTAAGGACACAGCACACAACCACCCGACCTG
TTCCCGACACCTCCCGGCTGCCTGGGGCCACCCCTGGGCTCACCACGGTGGAGATAGTGA
CAATGTCTCACCAAGCTCTGGGCGACGTTGCTGGCAGAGGAAATGAGAAGAAGCCCAGT
AGCGTGAGGGCTCTGTCCATTGTCCTCCCCATCGTGCTCCTCGTCTTCCTTTGCCTGGGGG
TCTTCCTTCTATGGAAGAACTGGCGGCTTAAGAACATCAACAGCATCAACTTTGACAACC
CCGTCTATCAGAAGACCACAGAGGATGAGGTCCACATTTGCCACAACCAGGACGGCTAC
AGCTACCCCTCGATGGTCAGTCTGGAGGATGACGTGGCGTGAACATCTGCCTGGAGTCCC
GTCCCTGCCCAGAACCCTTCCTGAGACCTCGCCGGCCTTGTTTTATTCAAAGACAGAGAA
GACCAAAGCATTGCCTGCCAGAGCTTTGTTTTATATATTTATTCATCTGGGAGGCAGAAC
AGGCTTCGGACAGTGCCCATGCAATGGCTTGGGTTGGGATTTTGGTTTCTTCCTTTCCTCG
TGAAGGATAAGAGAAACAGGCCCGGGGGGACCAGGATGACACCTCCATTTCTCTCCAGG
AAGTTTTGAGTTTCTCTCCACCGTGACACAATCCTCAAACATGGAAGATGAAAGGGGAG
GGGATGTCAGGCCCAGAGAAGCAAGTGGCTTTCAACACACAACAGCAGATGGCACCAAC
GGGACCCCCTGGCCCTGCCTCATCCACCAATCTCTAAGCCAAACCCCTAAACTCAGGAGT
CAACGTGTTTACCTCTTCTATGCAAGCCTTGCTAGACAGCCAGGTTAGCCTTTGCCCTGTC
ACCCCCGAATCATGACCCACCCAGTGTCTTTCGAGGTGGGTTTGTACCTTCCTTAAGCCA
GGAAAGGGATTCATGGCGTCGGAAATGATCTGGCTGAATCCGTGGTGGCACCGAGACCA
AACTCATTCACCAAATGATGCCACTTCCCAGAGGCAGAGCCTGAGTCACTGGTCACCCTT
AATATTTATTAAGTGCCTGAGACACCCGGTTACCTTGGCCGTGAGGACACGTGGCCTGCA
CCCAGGTGTGGCTGTCAGGACACCAGCCTGGTGCCCATCCTCCCGACCCCTACCCACTTC
CATTCCCGTGGTCTCCTTGCACTTTCTCAGTTCAGAGTTGTACACTGTGTACATTTGGCAT
TTGTGTTATTATTTTGCACTGTTTTCTGTCGTGTGTGTTGGGATGGGATCCCAGGCCAGGG
AAAGCCCGTGTCAATGAATGCCGGGGACAGAGAGGGGCAGGTTGACCGGGACTTCAAA
GCCGTGATCGTGAATATCGAGAACTGCCATTGTCGTCTTTATGTCCGCCCACCTAGTGCTT
CCACTTCTATGCAAATGCCTCCAAGCCATTCACTTCCCCAATCTTGTCGTTGATGGGTATG
TGTTTAAAACATGCACGGTGAGGCCGGGCGCAGTGGCTCACGCCTGTAATCCCAGCACTT
TGGGAGGCCGAGGCGGGTGGATCATGAGGTCAGGAGATCGAGACCATCCTGGCTAACAC
GTGAAACCCCGTCTCTACTAAAAATACAAAAAATTAGCCGGGCGTGGTGGCGGGCACCT
GTAGTCCCAGCTACTCGGGAGGCTGAGGCAGGAGAATGGTGTGAACCCGGGAAGCGGA
GCTTGCAGTGAGCCGAGATTGCGCCACTGCAGTCCGCAGTCTGGCCTGGGCGACAGAGC
GAGACTCCGTCTCAAAAAAAAAAAACAAAAAAAAACCATGCATGGTGCATCAGCAGCCC
ATGGCCTCTGGCCAGGCATGGCGAGGCTGAGGTGGGAGGATGGTTTGAGCTCAGGCATT
TGAGGCTGTCGTGAGCTATGATTATGCCACTGCTTTCCAGCCTGGGCAACATAGTAAGAC
CCCATCTCTTAAAAAATGAATTTGGCCAGACACAGGTGCCTCACGCCTGTAATCCCAGCA
CTTTGGGAGGCTGAGCTGGATCACTTGAGTTCAGGAGTTGGAGACCAGGCCTGAGCAAC
AAAGCGAGATCCCATCTCTACAAAAACCAAAAAGTTAAAAATCAGCTGGGTACGGTGGC
ACGTGCCTGTGATCCCAGCTACTTGGGAGGCTGAGGCAGGAGGATCGCCTGAGCCCAGG
AGGTGGAGGTTGCAGTGAGCCATGATCGAGCCACTGCACTCCAGCCTGGGCAACAGATG
AAGACCCTATTTCAGAAATACAACTATAAAAAAATAAATAAATCCTCCAGTCTGGATCGT
TTGACGGGACTTCAGGTTCTTTCTGAAATCGCCGTGTTACTGTTGCACTGATGTCCGGAG
AGACAGTGACAGCCTCCGTCAGACTCCCGCGTGAAGATGTCACAAGGGATTGGCAATTG
TCCCCAGGGACAAAACACTGTGTCCCCCCCAGTGCAGGGAACCGTGATAAGCCTTTCTGG
TTTCGGAGCACGTAAATGCGTCCCTGTACAGATAGTGGGGATTTTTTGTTATGTTTGCACT
TTGTATATTGGTTGAAACTGTTATCACTTATATATATATATATACACACATATATATAAAA
TCTATTTATTTTTGCAAACCCTGGTTGCTGTATTTGTTCAGTGACTATTCTCGGGGCCCTG
TGTAGGGGGTTATTGCCTCTGAAATGCCTCTTCTTTATGTACAAAGATTATTTGCACGAAC
TGGACTGTGTGCAACGCTTTTTGGGAGAATGATGTCCCCGTTGTATGTATGAGTGGCTTC
TGGGAGATGGGTGTCACTTTTTAAACCACTGTATAGAAGGTTTTTGTAGCCTGAATGTCT
TACTGTGATCAATTAAATTTCTTAAATGAACCAA.

In some embodiments, the agent, e.g., RNAi molecule and/or composition disclosed herein targets an LDLR-encoding transcript. In some embodiments, the LDLR-encoding transcript comprises a sequence that is at least 80%, 85%, 90%, 91%, 92%, 93%, 94%, 95%, 96%, 97%, 98%, 99% or 100% identical to the sequence of SEQ ID NO: 352, or a reverse complement thereof. In some embodiments, the agent, e.g., RNAi molecule and/or composition disclosed herein targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 352, or a reverse complement thereof. In some embodiments, the agent, e.g., RNAi molecule and/or composition disclosed herein targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 352, or a reverse complement thereof SEQ ID NO: 352:

GTGCAATCGCGGGAAGCCAGGGTTTCCAGCTAGGACACAGCAGGTCGTGATCCGGGTCG
GGACACTGCCTGGCAGAGGCTGCGAGCATGGGGCCCTGGGGCTGGAAATTGCGCTGGAC
CGTCGCCTTGCTCCTCGCCGCGGCGGGGACTGCAGTGGGCGACAGATGCGAAAGAAACG
AGTTCCAGTGCCAAGACGGGAAATGCATCTCCTACAAGTGGGTCTGCGATGGCAGCGCT
GAGTGCCAGGATGGCTCTGATGAGTCCCAGGAGACGTGCTTGTCTGTCACCTGCAAATCC
GGGGACTTCAGCTGTGGGGGCCGTGTCAACCGCTGCATTCCTCAGTTCTGGAGGTGCGAT
GGCCAAGTGGACTGCGACAACGGCTCAGACGAGCAAGGCTGTCCCCCCAAGACGTGCTC
CCAGGACGAGTTTCGCTGCCACGATGGGAAGTGCATCTCTCGGCAGTTCGTCTGTGACTC
AGACCGGGACTGCTTGGACGGCTCAGACGAGGCCTCCTGCCCGGTGCTCACCTGTGGTCC
CGCCAGCTTCCAGTGCAACAGCTCCACCTGCATCCCCCAGCTGTGGGCCTGCGACAACGA
CCCCGACTGCGAAGATGGCTCGGATGAGTGGCCGCAGCGCTGTAGGGGTCTTTACGTGTT
CCAAGGGGACAGTAGCCCCTGCTCGGCCTTCGAGTTCCACTGCCTAAGTGGCGAGTGCAT
CCACTCCAGCTGGCGCTGTGATGGTGGCCCCGACTGCAAGGACAAATCTGACGAGGAAA
ACTGCGCTGTGGCCACCTGTCGCCCTGACGAATTCCAGTGCTCTGATGGAAACTGCATCC
ATGGCAGCCGGCAGTGTGACCGGGAATATGACTGCAAGGACATGAGCGATGAAGTTGGC
TGCGTTAATGTGACACTCTGCGAGGGACCCAACAAGTTCAAGTGTCACAGCGGCGAATG
CATCACCCTGGACAAAGTCTGCAACATGGCTAGAGACTGCCGGGACTGGTCAGATGAAC
CCATCAAAGAGTGCGGGACCAACGAATGCTTGGACAACAACGGCGGCTGTTCCCACGTC
TGCAATGACCTTAAGATCGGCTACGAGTGCCTGTGCCCCGACGGCTTCCAGCTGGTGGCC
CAGCGAAGATGCGAAGATATCGATGAGTGTCAGGATCCCGACACCTGCAGCCAGCTCTG
CGTGAACCTGGAGGGTGGCTACAAGTGCCAGTGTGAGGAAGGCTTCCAGCTGGACCCCC
ACACGAAGGCCTGCAAGGCTGTGGGCTCCATCGCCTACCTCTTCTTCACCAACCGGCACG
AGGTCAGGAAGATGACGCTGGACCGGAGCGAGTACACCAGCCTCATCCCCAACCTGAGG
AACGTGGTCGCTCTGGACACGGAGGTGGCCAGCAATAGAATCTACTGGTCTGACCTGTCC
CAGAGAATGATCTGCAGCACCCAGCTTGACAGAGCCCACGGCGTCTCTTCCTATGACACC
GTCATCAGCAGAGACATCCAGGCCCCCGACGGGCTGGCTGTGGACTGGATCCACAGCAA
CATCTACTGGACCGACTCTGTCCTGGGCACTGTCTCTGTTGCGGATACCAAGGGCGTGAA
GAGGAAAACGTTATTCAGGGAGAACGGCTCCAAGCCAAGGGCCATCGTGGTGGATCCTG
TTCATGGCTTCATGTACTGGACTGACTGGGGAACTCCCGCCAAGATCAAGAAAGGGGGC
CTGAATGGTGTGGACATCTACTCGCTGGTGACTGAAAACATTCAGTGGCCCAATGGCATC
ACCCTAGATCTCCTCAGTGGCCGCCTCTACTGGGTTGACTCCAAACTTCACTCCATCTCAA
GCATCGATGTCAACGGGGGCAACCGGAAGACCATCTTGGAGGATGAAAAGAGGCTGGCC
CACCCCTTCTCCTTGGCCGTCTTTGAGGACAAAGTATTTTGGACAGATATCATCAACGAA
GCCATTTTCAGTGCCAACCGCCTCACAGGTTCCGATGTCAACTTGTTGGCTGAAAACCTA
CTGTCCCCAGAGGATATGGTTCTCTTCCACAACCTCACCCAGCCAAGAGGAGTGAACTGG
TGTGAGAGGACCACCCTGAGCAATGGCGGCTGCCAGTATCTGTGCCTCCCTGCCCCGCAG
ATCAACCCCCACTCGCCCAAGTTTACCTGCGCCTGCCCGGACGGCATGCTGCTGGCCAGG
GACATGAGGAGCTGCCTCACAGAGGCTGAGGCTGCAGTGGCCACCCAGGAGACATCCAC
CGTCAGGCTAAAGGTCAGCTCCACAGCCGTAAGGACACAGCACACAACCACCCGACCTG
TTCCCGACACCTCCCGGCTGCCTGGGGCCACCCCTGGGCTCACCACGGTGGAGATAGTGA
CAATGTCTCACCAAGTGCTCCTCGTCTTCCTTTGCCTGGGGGTCTTCCTTCTATGGAAGAA
CTGGCGGCTTAAGAACATCAACAGCATCAACTTTGACAACCCCGTCTATCAGAAGACCA
CAGAGGATGAGGTCCACATTTGCCACAACCAGGACGGCTACAGCTACCCCTCGAGACAG
ATGGTCAGTCTGGAGGATGACGTGGCGTGAACATCTGCCTGGAGTCCCGTCCCTGCCCAG
AACCCTTCCTGAGACCTCGCCGGCCTTGTTTTATTCAAAGACAGAGAAGACCAAAGCATT
GCCTGCCAGAGCTTTGTTTTATATATTTATTCATCTGGGAGGCAGAACAGGCTTCGGACA
GTGCCCATGCAATGGCTTGGGTTGGGATTTTGGTTTCTTCCTTTCCTCGTGAAGGATAAGA
GAAACAGGCCCGGGGGGACCAGGATGACACCTCCATTTCTCTCCAGGAAGTTTTGAGTTT
CTCTCCACCGTGACACAATCCTCAAACATGGAAGATGAAAGGGGAGGGGATGTCAGGCC
CAGAGAAGCAAGTGGCTTTCAACACACAACAGCAGATGGCACCAACGGGACCCCCTGGC
CCTGCCTCATCCACCAATCTCTAAGCCAAACCCCTAAACTCAGGAGTCAACGTGTTTACC
TCTTCTATGCAAGCCTTGCTAGACAGCCAGGTTAGCCTTTGCCCTGTCACCCCCGAATCAT
GACCCACCCAGTGTCTTTCGAGGTGGGTTTGTACCTTCCTTAAGCCAGGAAAGGGATTCA
TGGCGTCGGAAATGATCTGGCTGAATCCGTGGTGGCACCGAGACCAAACTCATTCACCA
AATGATGCCACTTCCCAGAGGCAGAGCCTGAGTCACTGGTCACCCTTAATATTTATTAAG
TGCCTGAGACACCCGGTTACCTTGGCCGTGAGGACACGTGGCCTGCACCCAGGTGTGGCT
GTCAGGACACCAGCCTGGTGCCCATCCTCCCGACCCCTACCCACTTCCATTCCCGTGGTC
TCCTTGCACTTTCTCAGTTCAGAGTTGTACACTGTGTACATTTGGCATTTGTGTTATTATTT
TGCACTGTTTTCTGTCGTGTGTGTTGGGATGGGATCCCAGGCCAGGGAAAGCCCGTGTCA
ATGAATGCCGGGGACAGAGAGGGGCAGGTTGACCGGGACTTCAAAGCCGTGATCGTGAA
TATCGAGAACTGCCATTGTCGTCTTTATGTCCGCCCACCTAGTGCTTCCACTTCTATGCAA
ATGCCTCCAAGCCATTCACTTCCCCAATCTTGTCGTTGATGGGTATGTGTTTAAAACATGC
ACGGTGAGGCCGGGCGCAGTGGCTCACGCCTGTAATCCCAGCACTTTGGGAGGCCGAGG
CGGGTGGATCATGAGGTCAGGAGATCGAGACCATCCTGGCTAACACGTGAAACCCCGTC
TCTACTAAAAATACAAAAAATTAGCCGGGCGTGGTGGCGGGCACCTGTAGTCCCAGCTA
CTCGGGAGGCTGAGGCAGGAGAATGGTGTGAACCCGGGAAGCGGAGCTTGCAGTGAGC
CGAGATTGCGCCACTGCAGTCCGCAGTCTGGCCTGGGCGACAGAGCGAGACTCCGTCTC
AAAAAAAAAAAACAAAAAAAAACCATGCATGGTGCATCAGCAGCCCATGGCCTCTGGCC
AGGCATGGCGAGGCTGAGGTGGGAGGATGGTTTGAGCTCAGGCATTTGAGGCTGTCGTG
AGCTATGATTATGCCACTGCTTTCCAGCCTGGGCAACATAGTAAGACCCCATCTCTTAAA
AAATGAATTTGGCCAGACACAGGTGCCTCACGCCTGTAATCCCAGCACTTTGGGAGGCTG
AGCTGGATCACTTGAGTTCAGGAGTTGGAGACCAGGCCTGAGCAACAAAGCGAGATCCC
ATCTCTACAAAAACCAAAAAGTTAAAAATCAGCTGGGTACGGTGGCACGTGCCTGTGAT
CCCAGCTACTTGGGAGGCTGAGGCAGGAGGATCGCCTGAGCCCAGGAGGTGGAGGTTGC
AGTGAGCCATGATCGAGCCACTGCACTCCAGCCTGGGCAACAGATGAAGACCCTATTTC
AGAAATACAACTATAAAAAAATAAATAAATCCTCCAGTCTGGATCGTTTGACGGGACTT
CAGGTTCTTTCTGAAATCGCCGTGTTACTGTTGCACTGATGTCCGGAGAGACAGTGACAG
CCTCCGTCAGACTCCCGCGTGAAGATGTCACAAGGGATTGGCAATTGTCCCCAGGGACA
AAACACTGTGTCCCCCCCAGTGCAGGGAACCGTGATAAGCCTTTCTGGTTTCGGAGCACG
TAAATGCGTCCCTGTACAGATAGTGGGGATTTTTTGTTATGTTTGCACTTTGTATATTGGT
TGAAACTGTTATCACTTATATATATATATATACACACATATATATAAAATCTATTTATTTT
TGCAAACCCTGGTTGCTGTATTTGTTCAGTGACTATTCTCGGGGCCCTGTGTAGGGGGTT
ATTGCCTCTGAAATGCCTCTTCTTTATGTACAAAGATTATTTGCACGAACTGGACTGTGTG
CAACGCTTTTTGGGAGAATGATGTCCCCGTTGTATGTATGAGTGGCTTCTGGGAGATGGG
TGTCACTTTTTAAACCACTGTATAGAAGGTTTTTGTAGCCTGAATGTCTTACTGTGATCAA
TTAAATTTCTTAAATGAACCAA.

In some embodiments, binding to low-density lipoprotein receptor (LDLR) is blocked by a composition comprising an agent that blocks low-density lipoprotein (LDL) binding to LDLR and/or that prevents LNP uptake into liver cells or tissue. In some embodiments, the LDLR comprises an amino acid sequence that is at least 85%, 90%, 91%, 92%, 93%, 94%, 95%, 96%, 97%, 98%, 99% or 100% identical to the sequence of SEQ ID NO: 353. SEQ ID NO: 353:

MGPWGWKLRWTVALLLAAAGTAVGDRCERNEFQCQDGKCISYKWVCDGSAECQDGSDES
QETCLSVTCKSGDFSCGGRVNRCIPQFWRCDGQVDCDNGSDEQGCPPKTCSQDEFRCHDGK
CISRQFVCDSDRDCLDGSDEASCPVLTCGPASFQCNSSTCIPQLWACDNDPDCEDGSDEWPQ
RCRGLYVFQGDSSPCSAFEFHCLSGECIHSSWRCDGGPDCKDKSDEENCAVATCRPDEFQCS
DGNCIHGSRQCDREYDCKDMSDEVGCVNVTLCEGPNKFKCHSGECITLDKVCNMARDCRD
WSDEPIKECGTNECLDNNGGCSHVCNDLKIGYECLCPDGFQLVAQRRCEDIDECQDPDTCSQ
LCVNLEGGYKCQCEEGFQLDPHTKACKAVGSIAYLFFTNRHEVRKMTLDRSEYTSLIPNLRN
VVALDTEVASNRIYWSDLSQRMICSTQLDRAHGVSSYDTVISRDIQAPDGLAVDWIHSNIYW
TDSVLGTVSVADTKGVKRKTLFRENGSKPRAIVVDPVHGFMYWTDWGTPAKIKKGGLNGV
DIYSLVTENIQWPNGITLDLLSGRLYWVDSKLHSISSIDVNGGNRKTILEDEKRLAHPFSLAVF
EDKVFWTDIINEAIFSANRLTGSDVNLLAENLLSPEDMVLFHNLTQPRGVNWCERTTLSNGG
CQYLCLPAPQINPHSPKFTCACPDGMLLARDMRSCLTEAEAAVATQETSTVRLKVSSTAVRT
QHTTTRPVPDTSRLPGATPGLTTVEIVTMSHQALGDVAGRGNEKKPSSVRALSIVLPIATELG
LWSRGQLCDRACLSLQCSSSSFAWGSSFYGRTGGLRTSTASTLTTPSIRRPQRMRSTFATTRT
ATATPRDRWSVWRMTWREHLPGVPSLPRTLPETSPALFYSKTEKTKALPARALFYIFIHLGGR
TGFGQCPCNGLGWDFGFFLSS.

In some embodiments, binding to low-density lipoprotein receptor (LDLR) is blocked by a composition comprising an agent that blocks low-density lipoprotein (LDL) binding to LDLR and/or that prevents LNP uptake into liver cells or tissue. In some embodiments, the LDLR comprises an amino acid sequence that is at least 85%, 90%, 91%, 92%, 93%, 94%, 95%, 96%, 97%, 98%, 99% or 100% identical to the sequence of SEQ ID NO: 354. SEQ ID NO: 354:

MGPWGWKLRWTVALLLAAAGTAVGDRCERNEFQCQDGKCISYKWVCDGSAECQDGSDES
QETCLSVTCKSGDFSCGGRVNRCIPQFWRCDGQVDCDNGSDEQGCPPKTCSQDEFRCHDGK
CISRQFVCDSDRDCLDGSDEASCPVLTCGPASFQCNSSTCIPQLWACDNDPDCEDGSDEWPQ
RCRGLYVFQGDSSPCSAFEFHCLSGECIHSSWRCDGGPDCKDKSDEENCAVATCRPDEFQCS
DGNCIHGSRQCDREYDCKDMSDEVGCVNVTLCEGPNKFKCHSGECITLDKVCNMARDCRD
WSDEPIKECGTNECLDNNGGCSHVCNDLKIGYECLCPDGFQLVAQRRCEDIDECQDPDTCSQ
LCVNLEGGYKCQCEEGFQLDPHTKACKAVGSIAYLFFTNRHEVRKMTLDRSEYTSLIPNLRN
VVALDTEVASNRIYWSDLSQRMICSTQLDRAHGVSSYDTVISRDIQAPDGLAVDWIHSNIYW
TDSVLGTVSVADTKGVKRKTLFRENGSKPRAIVVDPVHGFMYWTDWGTPAKIKKGGLNGV
DIYSLVTENIQWPNGITLDLLSGRLYWVDSKLHSISSIDVNGGNRKTILEDEKRLAHPFSLAVF
EDKVFWTDIINEAIFSANRLTGSDVNLLAENLLSPEDMVLFHNLTQPRGVNWCERTTLSNGG
CQYLCLPAPQINPHSPKFTCACPDGMLLARDMRSCLTEAEAAVATQETSTVRLKVSSTAVRT
QHTTTRPVPDTSRLPGATPGLTTVEIVTMSHQALGDVAGRGNEKKPSSVRALSIVLPIVLLVFL
CLGVFLLWKNWRLKNINSINFDNPVYQKTTEDEVHICHNQDGYSYPSMVSLEDDVA.

In some embodiments, binding to low-density lipoprotein receptor (LDLR) is blocked by a composition comprising an agent that blocks low-density lipoprotein (LDL) binding to LDLR and/or that prevents LNP uptake into liver cells or tissue. In some embodiments, the LDLR comprises an amino acid sequence that is at least 85%, 90%, 91%, 92%, 93%, 94%, 95%, 96%, 97%, 98%, 99% or 100% identical to the sequence of SEQ ID NO: 355. SEQ ID NO: 355:

MGPWGWKLRWTVALLLAAAGTAVGDRCERNEFQCQDGKCISYKWVCDGSAECQDGSDES
QETCLSVTCKSGDFSCGGRVNRCIPQFWRCDGQVDCDNGSDEQGCPPKTCSQDEFRCHDGK
CISRQFVCDSDRDCLDGSDEASCPVLTCGPASFQCNSSTCIPQLWACDNDPDCEDGSDEWPQ
RCRGLYVFQGDSSPCSAFEFHCLSGECIHSSWRCDGGPDCKDKSDEENCAVATCRPDEFQCS
DGNCIHGSRQCDREYDCKDMSDEVGCVNVTLCEGPNKFKCHSGECITLDKVCNMARDCRD
WSDEPIKECGTNECLDNNGGCSHVCNDLKIGYECLCPDGFQLVAQRRCEDIDECQDPDTCSQ
LCVNLEGGYKCQCEEGFQLDPHTKACKAVGSIAYLFFTNRHEVRKMTLDRSEYTSLIPNLRN
VVALDTEVASNRIYWSDLSQRMICSTQLDRAHGVSSYDTVISRDIQAPDGLAVDWIHSNIYW
TDSVLGTVSVADTKGVKRKTLFRENGSKPRAIVVDPVHGFMYWTDWGTPAKIKKGGLNGV
DIYSLVTENIQWPNGITLDLLSGRLYWVDSKLHSISSIDVNGGNRKTILEDEKRLAHPFSLAVF
EDKVFWTDIINEAIFSANRLTGSDVNLLAENLLSPEDMVLFHNLTQPRGVNWCERTTLSNGG
CQYLCLPAPQINPHSPKFTCACPDGMLLARDMRSCLTEAEAAVATQETSTVRLKVSSTAVRT
QHTTTRPVPDTSRLPGATPGLTTVEIVTMSHQVLLVFLCLGVFLLWKNWRLKNINSINFDNPV
YQKTTEDEVHICHNQDGYSYPSRQMVSLEDDVA.

In some embodiments, the agent, e.g., RNAi molecule and/or composition disclosed herein comprises an siRNA that is capable of knocking down LDLR. In some embodiments, the siRNA comprises a ribonucleotide sequence at least 80% identical to a ribonucleotide sequence from the LDLR. Preferably, the siRNA is at least 90%, 95%, 96%, 97%, 98%, 99% or 100% identical to the ribonucleotide sequence of the target. In some embodiments, the siRNA is 100% identical to the nucleotide sequence of the target. However, siRNA molecules with insertions, deletions or single point mutations relative to a target may also be effective. In some embodiments, the siRNA comprises a single strand. In some embodiments, the siRNA comprises a duplex siRNA. In some embodiments, the siRNA comprises a duplex siRNA comprising a sense (“passenger”) and antisense (“guide”) strand. In some embodiments, the siRNA targets LDLR in the liver. Tools to assist siRNA design are readily available to the public and are known in the art.

In some embodiments, the composition comprises an RNAi molecule that is between 18-31 nucleotides in length. In some embodiments, the RNAi (e.g., siRNA) is between 19-27 nucleotides in length. In some embodiments, the RNAi (e.g., siRNA) is between 19-25 nucleotides in length. In some embodiments, the RNAi (e.g., siRNA) is between 19-23 nucleotides in length. In some embodiments, the RNAi (e.g., siRNA) is between 19-21 nucleotides in length. In some embodiments, the RNAi (e.g., siRNA) is no more than 21, 25, or 31 nucleotides in length. In some embodiments, the RNAi (e.g., siRNA) is 21 nucleotides in length. In some embodiments, the RNAi is siRNA and comprises the sequence of any of the sequences in Table 3A (SEQ ID NOs: 4300-9964). In some embodiments, the RNA is siRNA and comprises the sequence of any one of SEQ ID NOs: 4300-4309 or 9965-9974. In some embodiments, the RNAi is a duplex siRNA and comprises a pair of sequences of any one of SEQ ID NOs: 4300 and 9965, 4301 and 9966, 4302 and 9967, 4303 and 9968, 4304 and 9969, 4305 and 9970, 4306 and 9971, 4307 and 9972, 4308 and 9973, or 4309 and 9974. In some embodiments, the siRNA comprises no more than and no fewer than 19 contiguous nucleotides of any of the sequences in Table 3A or 3B. In some embodiments, the siRNA comprises no more than and no fewer than 19 contiguous nucleotides of any of the sequences of any one of SEQ ID NOs: 4300-4309 or 9965-9974. In some embodiments, the RNAi is a duplex siRNA and comprises a pair of sequences wherein each sequence comprises no more than and no fewer than 19 contiguous nucleotides of any of the sequences of SEQ ID NOs: 4300 and 9965, 4301 and 9966, 4302 and 9967, 4303 and 9968, 4304 and 9969, 4305 and 9970, 4306 and 9971, 4307 and 9972, 4308 and 9973, or 4309 and 9974. In some embodiments, the siRNA comprises no more than and no fewer than 20 contiguous nucleotides of any of the sequences in Table 3A or 3B. In some embodiments, the siRNA comprises no more than and no fewer than 20 contiguous nucleotides of any of the sequences of any one of SEQ ID NOs: 4300-4309 or 9965-9974. In some embodiments, the RNAi is a duplex siRNA and comprises a pair of sequences wherein each sequence comprises no more than and no fewer than 20 contiguous nucleotides of any of the sequences of SEQ ID NOs: 4300 and 9965, 4301 and 9966, 4302 and 9967, 4303 and 9968, 4304 and 9969, 4305 and 9970, 4306 and 9971, 4307 and 9972, 4308 and 9973, or 4309 and 9974. In some embodiments, the siRNA comprises no more than and no fewer than 21 contiguous nucleotides of any of the sequences in Table 3A or 3B. In some embodiments, the siRNA comprises no more than and no fewer than 21 contiguous nucleotides of any of the sequences of any one of SEQ ID NOs: 4300-4309 or 9965-9974. In some embodiments, the RNAi is a duplex siRNA and comprises a pair of sequences wherein each sequence comprises no more than and no fewer than 21 contiguous nucleotides of any of the sequences of SEQ ID NOs: 4300 and 9965, 4301 and 9966, 4302 and 9967, 4303 and 9968, 4304 and 9969, 4305 and 9970, 4306 and 9971, 4307 and 9972, 4308 and 9973, or 4309 and 9974. SEQ ID NOs: 4300-9964 in Table 3A reflect exemplary sequences for the sense or antisense 5′ to 3′ strands of siRNA.

In some embodiments the composition comprises a RNAi molecule (e.g., siRNA) that comprises between 13-21 consecutive nucleic acids of any one of the nucleic acid sequences in Table 3A or 3B. In some embodiments, the RNAi (e.g., siRNA) comprises between 14-21 consecutive nucleic acids of any one of the nucleic acid sequences in Table 3. In some embodiments, the RNAi (e.g., siRNA) comprises a sequence that is at least 80%, 85%, 90%, 91%, 92%, 93%, 94%, 95%, 96%, 97%, 98%, 99% or 100% identical to any one of the nucleic acid sequences of Table 3A or 3B. In some embodiments, the RNAi (e.g., siRNA) comprises a sequence that is at least 80%, 85%, 90%, 91%, 92%, 93%, 94%, 95%, 96%, 97%, 98%, 99% or 100% identical to any one of SEQ ID NOs: 4300-4309 or 9965-9974. In some embodiments, the RNAi (e.g., siRNA) comprises a pair of sequences wherein each sequence is at least 80%, 85%, 90%, 91%, 92%, 93%, 94%, 95%, 96%, 97%, 98%, 99% or 100% identical to each nucleic acid sequence of the pair of SEQ ID NOs: 4300 and 9965, 4301 and 9966, 4302 and 9967, 4303 and 9968, 4304 and 9969, 4305 and 9970, 4306 and 9971, 4307 and 9972, 4308 and 9973, or 4309 and 9974. In some embodiments, the RNAi (e.g., siRNA) comprises at least 13, at least 14, at least 15, at least 16, at least 17, at least 18, at least 19, at least 20, or at least 21 consecutive nucleic acids of a sequence that is at least 80%, 85%, 90%, 91%, 92%, 93%, 94%, 95%, 96%, 97%, 98%, 99% or 100% identical to any one of the nucleic acid sequences of Table 3A or 3B. In some embodiments, the RNAi (e.g., siRNA) comprises at least 13, at least 14, at least 15, at least 16, at least 17, at least 18, at least 19, at least 20, or at least 21 consecutive nucleic acids of a sequence that is at least 80%, 85%, 90%, 91%, 92%, 93%, 94%, 95%, 96%, 97%, 98%, 99% or 100% identical to any one of SEQ ID NOs: 4300-4309 or 9965-9974. In some embodiments, the RNAi (e.g., siRNA) comprises a pair of sequences wherein each sequence comprises at least 13, at least 14, at least 15, at least 16, at least 17, at least 18, at least 19, at least 20, or at least 21 consecutive nucleic acids of a sequence that is at least 80%, 85%, 90%, 91%, 92%, 93%, 94%, 95%, 96%, 97%, 98%, 99% or 100% identical to each nucleic acid sequence of the pair of SEQ ID NOs: 4300 and 9965, 4301 and 9966, 4302 and 9967, 4303 and 9968, 4304 and 9969, 4305 and 9970, 4306 and 9971, 4307 and 9972, 4308 and 9973, or 4309 and 9974. In some embodiments, the RNAi (e.g., siRNA) comprises at least 13, at least 14, at least 15, at least 16, at least 17, at least 18, at least 19, at least 20, or at least 21 consecutive nucleic acids of a sequence that is identical to any one of the nucleic acid sequences of Table 3A or 3B. In some embodiments, the RNAi (e.g., siRNA) comprises at least 13, at least 14, at least 15, at least 16, at least 17, at least 18, at least 19, at least 20, or at least 21 consecutive nucleic acids of a sequence that is identical to any one of SEQ ID NOs: 4300-4309 or 9965-9974. In some embodiments, the RNAi (e.g., siRNA) comprises a pair of sequences wherein each sequence comprises at least 13, at least 14, at least 15, at least 16, at least 17, at least 18, at least 19, at least 20, or at least 21 consecutive nucleic acids of a sequence that is identical to each nucleic acid sequence of the pair of SEQ ID NOs: 4300 and 9965, 4301 and 9966, 4302 and 9967, 4303 and 9968, 4304 and 9969, 4305 and 9970, 4306 and 9971, 4307 and 9972, 4308 and 9973, or 4309 and 9974. In some embodiments, the RNAi (e.g., siRNA) targets 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to any one of the nucleic acid sequences of Table 3A or 3B. In some embodiments, the RNAi (e.g., siRNA) targets 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to any one of the nucleic acid sequences of SEQ ID NOs: 4300-4309 or SEQ ID NOs: 9965-9974. In some embodiments, the RNAi (e.g., siRNA) comprises a pair of sequences wherein each sequence targets 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to each nucleic acid sequence of the pair of SEQ ID NOs: 4300 and 9965, 4301 and 9966, 4302 and 9967, 4303 and 9968, 4304 and 9969, 4305 and 9970, 4306 and 9971, 4307 and 9972, 4308 and 9973, or 4309 and 9974. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4300. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4301. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4302. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4303. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4304. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4305. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4306. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4307. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4308. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 4309. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9965. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9966. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9967. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9968. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9969. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9970. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9971. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9972. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9973. In some embodiments, the RNAi (e.g., siRNA) comprises 13-19, 13-20, 13-21, 13-22, 13-23, 13-24, 13-25, 14-19, 14-20, 14-21, 14-22, 14-23, 14-24, or 14-25 consecutive nucleic acids of a sequence that is at least 80%, 85%, 90%, 91%, 92%, 93%, 94%, 95%, 96%, 97%, 98%, 99% or 100% identical to SEQ ID NO: 9974.

In some embodiments, the LDLR-encoding transcript comprises a sequence that is at least 80%, 85%, 90%, 91%, 92%, 93%, 94%, 95%, 96%, 97%, 98%, 99% or 100% identical to the sequence of SEQ ID NO: 350, or a reverse complement thereof. In some embodiments, the RNAi (e.g., siRNA) targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 350, or a reverse complement thereof. In some embodiments, the RNAi (e.g., siRNA) targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 350, or a reverse complement thereof.

In some embodiments, the LDLR-encoding transcript comprises a sequence that is at least 80%, 85%, 90%, 91%, 92%, 93%, 94%, 95%, 96%, 97%, 98%, 99% or 100% identical to the sequence of SEQ ID NO: 351, or a reverse complement thereof. In some embodiments, the RNAi molecule targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 351, or a reverse complement thereof. In some embodiments, the RNAi molecule targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 351, or a reverse complement thereof.

In some embodiments, the LDLR-encoding transcript comprises a sequence that is at least 80%, 85%, 90%, 91%, 92%, 93%, 94%, 95%, 96%, 97%, 98%, 99% or 100% identical to the sequence of SEQ ID NO: 352, or a reverse complement thereof. In some embodiments, the RNAi molecule targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 352, or a reverse complement thereof. In some embodiments, the RNAi molecule targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 352, or a reverse complement thereof.

In some embodiments, the RNAi molecule is single-stranded. In some embodiments, the RNAi molecule is double-stranded. It should be noted that, any nucleotide lengths of any RNAi molecules recited in this application refer to a single strand of the RNAi molecule, even if that single strand is a member of a double-stranded RNAi molecule. For example, if an RNAi molecule is 21 nucleotides in length and is double stranded (without overhangs), the molecule would comprise a total of 42 nucleotides (21 nucleotides in each strand). In some embodiments, the RNAi molecule is double stranded and comprises blunt ends. In some embodiments, the RNAi molecule is double-stranded and comprises overhangs of one or more nucleotides. In some embodiments, the RNAi molecule is double stranded for only a portion of the molecule. For example, in some embodiments, a double-stranded RNAi molecule comprises overhangs on the sense and/or antisense strand of 1, 2, 3, 4, or 5 or more nucleotides. In some embodiments, a double-stranded RNAi molecule comprises overhangs on the sense and/or antisense strand of 1, 2 or 3 nucleotides.

In some embodiments, any of the RNAi molecules (e.g., siRNA) disclosed herein comprises a nucleotide sequence that shares complementarity (e.g., 100% complementarity) with a target sequence in an LDLR RNA transcript. In some embodiments, the RNAi molecule comprises at least 17, 18, 19, 20, or 21 nucleotides that are complementary to a target sequence in an LDLR RNA transcript. In some embodiments, the RNAi molecule comprises at least 17, 18, 19, 20, or 21 nucleotides that are complementary to a target sequence in an LDLR RNA transcript, but wherein one or more nucleotides on the 3′ end of the RNAi molecule are not complementary to the target sequence in the LDLR RNA transcript. In some embodiments, the RNAi molecule comprises at least 17, 18, 19, 20, or 21 nucleotides that are complementary to a target sequence in an LDLR RNA transcript, but wherein one or more nucleotides on the 5′ end of the RNAi molecule are not complementary to the target sequence in the LDLR RNA transcript. In some embodiments, the RNAi molecule comprises at least 17, 18, 19, 20, or 21 nucleotides that are complementary to a target sequence in an LDLR RNA transcript, but wherein one or more nucleotides on the 3′ end and the 5′ end of the RNAi molecule are not complementary to the target sequence in the LDLR RNA transcript.

### B. Antisense Oligonucleotides as Agent

In some embodiments, antisense oligonucleotides (ASOs) are the agent used to block LDL binding to LDLR in the liver and/or for preventing LNP uptake into liver cells or tissue. The ASOs can be a single or double stranded DNA or RNA, or chimeric mixtures or derivatives or modified versions thereof. As is known in the art, for antisense oligonucleotides to sufficiently inhibit their target sequence as efficiently as possible, there should be a degree of complementarity between the antisense oligonucleotides and the corresponding target sequence. Chemical modifications of ASOs are known in the art to increase their resistance to various nucleases, as well as their binding affinity to RNA targets. Phosphorothioate (PS) modification, in which a non-bridging oxygen is replaced by a sulfur atom in the phosphate backbone; 2′-O-methyl (2′-O-Me), 2′-O-methoxyethyl (MOE) modification; constrained ethyl (cEt) modification; and bicyclic nucleoside modifications such as 2′,4′-methylene bridged nucleic acids, commonly called locked nucleic acid (LNA) modification, are non-limiting examples. Accordingly, in some embodiments, the antisense oligonucleotide comprises a modified sequence. In some embodiments, the ASO contains MOE or LNA modifications.

In some embodiments, the antisense oligonucleotide is linked to ligands or conjugates known in the art and/or described herein or delivered by non-viral tissue-specific delivery vehicles, which may be used, e.g., to increase the cellular uptake of antisense oligonucleotides.

In some embodiments, any of the ASOs disclosed herein are administered “naked”, i.e., without a molecule intended for a cell or tissue-specific delivery. For example, in some embodiments, the agent is administered “naked” in a pharmaceutically acceptable buffer, e.g., a buffered saline solution such as PBS.

In some embodiments, any of the ASOs disclosed herein is at least 14, 15, 16, 17, 18, 19, 20, 21, 22, 23, 24, or 25 nucleotides in length. In some embodiments, any of the ASOs disclosed herein is no more than 35, 34, 33, 32, 31, 30, 29, 28, 27, 26 or 25 nucleotides in length. In some embodiments, any of the ASOs disclosed herein is between 14-35, 14-30, 14-25, 14-20, 20-35, 20-30, 20-25, 25-35, or 25-30 nucleotides in length.

In some embodiments, the antisense oligonucleotide is administered without conjugation and without a non-viral tissue-specific delivery vehicle. In some embodiments, the antisense oligonucleotides are administered without a non-viral tissue-specific delivery vehicle and are administered in a composition comprising a pharmaceutically acceptable carrier.

In some embodiments, the antisense oligonucleotide targets an LDLR-encoding transcript. In some embodiments, the LDLR-encoding transcript comprises a sequence that is at least 80%, 85%, 90%, 91%, 92%, 93%, 94%, 95%, 96%, 97%, 98%, 99% or 100% identical to the sequence of SEQ ID NO: 350, or a reverse complement thereof. In some embodiments, the LDLR-encoding transcript comprises a sequence that is at least 80%, 85%, 90%, 91%, 92%, 93%, 94%, 95%, 96%, 97%, 98%, 99% or 100% identical to the sequence of SEQ ID NO: 351, or a reverse complement thereof. In some embodiments, the LDLR-encoding transcript comprises a sequence that is at least 80%, 85%, 90%, 91%, 92%, 93%, 94%, 95%, 96%, 97%, 98%, 99% or 100% identical to the sequence of SEQ ID NO: 352, or a reverse complement thereof.

In some embodiments, the antisense oligonucleotide targets 16, 17, 18, 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of any one of SEQ ID NOs: 350, 351, or 352, or a reverse complement thereof. In some embodiments, the antisense oligonucleotide targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 350, or a reverse complement thereof. In some embodiments, the antisense oligonucleotide targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 351, or a reverse complement thereof. In some embodiments, the antisense oligonucleotide targets 19, 20, 21, 22, 23, 24, 25, 26, 27, 28, 29, 30, 31 or 32 contiguous nucleotides of a sequence that is at least 80%, 85%, 90%, 91%, 92%, 93%, 94%, 95%, 96%, 97%, 98%, 99% or 100% identical to the sequence of SEQ ID NO: 352, or a reverse complement thereof.

In some embodiments, the antisense oligonucleotide targets 16-32 contiguous nucleotides of a sequence that is at least 80%, 85%, 90%, 91%, 92%, 93%, 94%, 95%, 96%, 97%, 98%, 99% or 100% identical to the sequence of any one of SEQ ID NOs: 350, 351, or 352, or a reverse complement thereof. In some embodiments, the antisense oligonucleotide targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 350, or a reverse complement thereof. In some embodiments, the antisense oligonucleotide targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 351, or a reverse complement thereof. In some embodiments, the antisense oligonucleotide targets 19-32, 19-25, 19-22, or 20-21 contiguous nucleotides of a sequence that is at least 80%, 85%, 90%, 91%, 92%, 93%, 94%, 95%, 96%, 97%, 98%, 99% or 100% identical to the sequence of SEQ ID NO: 352, or a reverse complement thereof.

### C. Non-RNAi Agents

In some embodiments, the agent that blocks LDL binding to LDLR and/or otherwise inhibit or reduce the interaction between LDL and the receptor and/or for preventing LNP uptake into liver cells or tissue is a small molecule, antibody, or soluble receptor. In some embodiments, the non-RNAi agent targets the LDLR.

### D. Agent Delivery

In some embodiments, the compositions comprise: a) an agent that blocks LDL binding to an LDL receptor (LDLR) and/or for prevents LNP uptake into liver cells or tissue and b) a delivery molecule that delivers the agent to the liver. In some embodiments, the delivery molecule comprises a lipid nanoparticle (LNP), a lipoplex, an adenovirus vector, or an AAV. In some embodiments, the delivery molecule comprises an AAV, and the AAV is an AAV1, AAV2, AAV3, AAV4, AAVS, AAV6, AAV7, AAV8, AAVrh10, AAVrh74, AAV9, AAV9P, or Myo-AAV. In some embodiments, the delivery molecule comprises a lipoplex. In some embodiments, the delivery molecule comprises an LNP. In some embodiments, the LNP is a liver-tropic LNP. In some embodiments, the LNP comprises LP-01. In some embodiments, the LNP comprise MC3. In some embodiments, the LNP comprises an ionizable lipid. In some embodiments, the LNP comprises a helper lipid (e.g., 1,2-distearoyl-sn-glycero-3-phosphorylcholine (DSPC) or N-(hexadecanoyl)-sphing-4-enine-1-phosphocholine (egg sphingomyelin [ESM])). In some embodiments, the LNP comprises a PEGylated-lipid. In some embodiments, the LNP comprises ionizable lipid/helper lipid/cholesterol/PEG-lipid in molar ratios of 50:10:38.5:1.5, respectively. In some embodiments, the LNP comprises ionizable lipid/helper lipid/cholesterol/PEG-lipid in molar ratios of 20:44.25:34.25:1.5 mol/mol, 30:38.5:30:1.5, 40:33:25.5:1.5, 50:27.25:21.25:1.5 mol/mol, or 55:24.5:19:1.5 mol/mol. In some embodiments, the LNP comprises DSPC. In some embodiments, the LNP comprises cholesterol. In some embodiments, the LNP comprises myristoyl diglyceride. In some embodiments, the LNP comprises PEG-ylated myristoyl diglyceride. In some embodiments, the LNP comprises DMG-PEG2000. In some embodiments, the LNP comprises LP-01 (BP-26809, BroadPharm), DSPC (850365, Avanti Polar Lipids), Cholesterol (C3045, Milipore Sigma), and DMG-PEG2000 (880151, Avanti Polar Lipids). In some embodiments, the LNP comprises about 50% LP-01, about 39% cholesterol, about 9% DSPC, and about 2% DMG-PEG2000. In some embodiments, the LNP comprises PEG-carbamoyl-1,2-dimyristyloxy-propylamine, DLin-DMA, DSPC, and cholesterol at a molar ratio of 2:40:10:4. In some embodiments, the LNP comprises lipidoid 98N12-5(1)4HCl, cholesterol, and mPEG2000-DMG at a molar ratio of 42:48:10. In some embodiments, the LNP comprises DLin-MC3-DMA, cholesterol, and mPEG2000-DMG at a molar ratio of 42:48:10. In some embodiments, the LNP is any of the LNPs described in Xu et al., 2023, ACS Nano, 17, 5, 4942-4957, which is incorporated herein in its entirety.

In some embodiments, the delivery molecule is conjugated to the agent (e.g., RNAi, ASO, or siRNA), and functions to deliver the agent to the liver. As used herein, “liver targeting moiety” includes but is not limited to, any conjugate that can be applied to any of the agents that block LDL binding to an LDLR (e.g., siRNAs, ASOs or RNAi) and/or for prevent LNP uptake into liver cells or tissue to enhance their delivery and/or uptake by the liver, including any known conjugates. Lipid moieties (e.g., lipid-conjugated siRNAs or ASOs), such as cholesterol-conjugated siRNAs or ASOs, and any other conjugates groups or moieties that are known in the art to effectively target the liver can also be used. In some embodiments, the delivery molecule comprises a lipid. In some embodiments, the compositions comprise lipid-conjugated agents (e.g., siRNAs or ASOs).

In some embodiments, the delivery molecule comprises at least one galactose or galactose derivative. In some embodiments, the compositions comprise siRNA conjugated to at least one galactose or galactose derivative. Galactose or galactose derivatives can target hepatocytes via their binding to the asialo glycoprotein receptor that is unique to and is highly expressed on the surface of hepatocytes (ASGPr). Binding of galactose moieties to ASGPr facilitates intracellular entry of the cell-specific target of the transferring polymer into the hepatocyte and the delivery polymer to the hepatocyte. Exemplary galactose or galactose derivatives include lactose, galactose, N-acetylgalactosamine (GalNAc), GalNAc-6 (doi: 10.1038/s41467-023-37465-1), galactosamine, N-formylgalactosamine, N-acetylgalactosamine, N-propionylgalactosamine, Nn-butanoylgalactosamine, and N-isobutanoyl-galactosamine (Iobst, S T and Drickamer, K. JBC 1996, 271, 6686). In some embodiments, the delivery molecule comprises N-acetylgalactosamine (GalNAc). In some embodiments, the delivery molecule comprises GalNAc-6. In some embodiments, the compositions comprise GalNAc-conjugated agents (e.g., siRNAs or ASOs).

As is also known in the art, the agent that blocks LDL binding to an LDLR and/or prevents LNP uptake into liver cells or tissue can be delivered by non-viral tissue-specific delivery vehicles. In some embodiments, the delivery molecule comprises nanoparticles, liposomes, ribonucleoproteins, positively charged peptides, small molecule RNA-conjugates, aptamer-RNA chimeras, and RNA-fusion protein complexes. In some embodiments, the delivery molecule comprises a lipid nanoparticle (LNP).

A LNP refers to any particle having a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm.

LNPs can be made from cationic, anionic, or neutral lipids. Neutral lipids, such as the fusogenic phospholipid DOPE or the membrane component cholesterol, can be included in LNPs as ‘helper lipids’ to enhance transfection activity and nanoparticle stability. Limitations of cationic lipids include low efficacy owing to poor stability and rapid clearance, as well as the generation of inflammatory or anti-inflammatory responses. LNPs can also be comprised of hydrophobic lipids, hydrophilic lipids, or both hydrophobic and hydrophilic lipids. Any lipid or combination of lipids that are known in the art can be used to produce an LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20. In particular embodiments, the LNP comprises DOSPA (2,3-dioleoyloxy-N-[2(sperminecarboxamido)ethyl]-N,N-dimethyl-1-propaniminium trifluoroacetate) and DOPE (dioleoylphosphatidylethanolamine). The lipids can be combined in any number of molar ratios to produce an LNP. In some embodiments, the LNP comprises a 3:1 mixture of DOSPA and DOPE. In addition, the polynucleotide(s) can be combined with lipid(s) in a wide range of molar ratios to produce an LNP. In some embodiments, the LNP is an “off-the-shelf” LNP, such as Lipofectamine.

### E. Payload

In some embodiments, the agent that blocks LDL binding to an LDLR and/or prevents LNP uptake into liver cells or tissue and that is delivered by a delivery molecule to the liver further comprises a payload. In some embodiments, the payload comprises a therapeutic agent. In some embodiments, the payload is administered with an LNP, or lipoplex. In some compositions, the payload is encapsulated in a lipid nanoparticle (LNP). In some embodiments, the payload is encapsulated in a lipoplex.

In some embodiments, administration of any of the agents that block LDL binding to LDLR and/or prevents LNP uptake into liver cells or tissue increases the percentage of payload delivered to a non-liver target. In some embodiments, administration of any of the agents that block LDL binding to LDLR and/or prevents LNP uptake into liver cells or tissue decreases the percentage of payload delivered to the liver. Accordingly, the present invention contemplates administering a single payload or multiple payloads, optionally in one or more LNPs, that have a non-liver target and/or compositions thereof. In particular embodiments, the agents that block LDL binding to the LDLR and/or that prevent LNP uptake into liver cells or tissue are administered separately from the payload (e.g., in separate LNPs).

In particular embodiments, the payload is associated with an LNP or lipoplex. Lipid nanoparticles (LNPs) and lipoplexes are a known means for delivery of nucleotide and protein cargo, and may be used for delivery of the payloads, guide RNAs, compositions, or pharmaceutical formulations disclosed herein. In some embodiments, the LNPs or lipoplexes deliver nucleic acid, protein, or nucleic acid together with protein.

In some embodiments, the payload comprises CRISPR-Cas components, any of which are known in the art. In some embodiments, the payload comprises a component of a CRISPR/Cas system or a nucleic acid encoding one or more component of a CRISPR/Cas system, a biologic, or a small molecule, optionally wherein the component of a CRISPR/Cas system comprises a nucleic acid encoding one or more a guide RNAs, one or more scaffolds, and/or one or more endonucleases. In some embodiments, the payload comprises a nucleic acid encoding a Cas9 protein. Such embodiments include for example, a payload comprising a nucleic acid encoding Streptococcus pyogenes (SpCas9), Staphylococcus aureus (SaCas9) and/or Staphylococcus lugdunensis (SluCas9) and further comprising a nucleic acid encoding one or more guide RNAs. In such embodiments, the nucleic acid encoding the Cas9 protein is under the control of a CK8e promoter. In some embodiments, the nucleic acid encoding the guide RNA sequence is under the control of a hU6c promoter. In some embodiments, in addition to guide RNA and Cas9 sequences, the one or more vectors further comprise nucleic acids that do not encode guide RNAs. Nucleic acids that do not encode guide RNA and Cas9 include, but are not limited to, promoters, enhancers, and regulatory sequences. In some embodiments, the payload comprises one or more nucleotide sequence(s) encoding a crRNA, a trRNA, or a crRNA and trRNA.

In some embodiments, the muscle-cell cell specific promoter is a variant of the CK8 promoter, called CK8e. In some embodiments, the size of the CK8e promoter is 436 bp. In some embodiments, the CK8e promoter comprises a nucleotide sequence that is at least 90%, 91%, 92%, 93%, 94%, 95%, 96%, 97%, 98%, 99% or 100% identical to the sequence of SEQ ID NO: 302:

| | |
|---|---|
| 1 | TGCCCATGTA AGGAGGCAAG GCCTGGGGAC ACCCGAGATG CCTGGTTATA ATTAACCCAG |
| 61 | ACATGTGGCT GCCCCCCCCC CCCCAACACC TGCTGCCTCT AAAAATAACC CTGCATGCCA |
| 121 | TGTTCCCGGC GAAGGGCCAG CTGTCCCCCG CCAGCTAGAC TCAGCACTTA GTTTAGGAAC |
| 181 | CAGTGAGCAA GTCAGCCCTT GGGGCAGCCC ATACAAGGCC ATGGGGCTGG GCAAGCTGCA |
| 241 | CGCCTGGGTC CGGGGTGGGC ACGGTGCCCG GGCAACGAGC TGAAAGCTCA TCTGCTCTCA |
| 361 | GGGGCCCCTC CCTGGGGACA GCCCCTCCTG GCTAGTCACA CCCTGTAGGC TCCTCTATAT |
| 361 | AACCCAGGGG CACAGGGGCT GCCCTCATTC TACCACCACC TCCACAGCAC AGACAGACAC |
| 421 | TCAGGAGCCA GCCAGC |

In some embodiments, the promoter for expression of any of the nucleic acids disclosed herein is a U6 promoter. In some embodiments, the U6 promoter is a hU6c promoter and comprises a nucleotide sequence that is at least 90%, 91%, 92%, 93%, 94%, 95%, 96%, 97%, 98%, 99% or 100% identical to the sequence of SEQ ID NO: 303:

GAGGGCCTATTTCCCATGATTCCTTCATATTTGCATATACGATACAAGG
CTGTTAGAGAGATAATTGGAATTAATTTGACTGTAAACACAAAGATATT
AGTACAAAATACGTGACGTAGAAAGTAATAATTTCTTGGGTAGTTTGCA
GTTTTAAAATTATGTTTTAAAATGGACTATCATATGCTTACCGTAACTT
GAAAGTATTTCGATTTCTTGGCTTTATATATCTTGTGGAAAGGACGAAA
CACC.

In some embodiments, the promoter for expression of any of the nucleic acids disclosed herein is a H1 promoter. In some embodiments, the H1 promoter comprises a nucleotide sequence that is at least 90%, 91%, 92%, 93%, 94%, 95%, 96%, 97%, 98%, 99% or 100% identical to the sequence of SEQ ID NO: 304:

| | |
|---|---|
| gctcggcgcg cccatatttg catgtcgcta tgtgttctgg gaaatcacca taaacgtgaa | 60 |
| atgtctttgg atttgggaat cttataagtt ctgtatgaga ccacggta | 108 |

In some embodiments, the promoter for expression of any of the nucleic acids disclosed herein is a 7SK2 promoter. In some embodiments, the 7SK promoter is a 7SK2 promoter and comprises a nucleotide sequence that is at least 90%, 91%, 92%, 93%, 94%, 95%, 96%, 97%, 98%, 99% or 100% identical to the sequence of SEQ ID NO: 305:

CTGCAGTATTTAGCATGCCCCACCCATCTGCAAGGCATTCTGGATAGTG
TCAAAACAGCCGGAAATCAAGTCCGTTTATCTCAAACTTTAGCATTTTG
GGAATAAATGATATTTGCTATGCTGGTTAAATTAGATTTTAGTTAAATT
TCCTGCTGAAGCTCTAGTACGATAAGCAACTTGACCTAAGTGTAAAGTT
GAGACTTCCTTCAGGTTTATATAGCTTGTGCGCCGCTTGGGTACCTC.

In some embodiments, the payload comprises one or more guide RNAs. In some embodiments, the guide RNA is chemically modified. A guide RNA comprising one or more modified nucleosides or nucleotides is called a “modified” guide RNA or “chemically modified” guide RNA, to describe the presence of one or more non-naturally and/or naturally occurring components or configurations that are used instead of or in addition to the canonical A, G, C, and U residues. A discussion of modified guide RNAs can be found in WO2022/056000, which is incorporated herein in its entirety. In some embodiments, the guide RNAs are unmodified.

In some embodiments, the payload comprises multiple nucleic acids encoding more than one guide RNA. In some embodiments, the payload comprises two nucleic acids encoding two guide RNA sequences.

In some embodiments, the payload comprises a nucleic acid encoding a Cas9 protein (e.g., an SaCas9 protein or SluCas9 protein), a nucleic acid encoding a first guide RNA, and a nucleic acid encoding a second guide RNA. In some embodiments, the payload does not comprise a nucleic acid encoding more than two guide RNAs. In some embodiments, the nucleic acid encoding the first guide RNA is the same as the nucleic acid encoding the second guide RNA. In some embodiments, the nucleic acid encoding the first guide RNA is different from the nucleic acid encoding the second guide RNA. In some embodiments, the payload comprises a single nucleic acid molecule, wherein the single nucleic acid molecule comprises a nucleic acid encoding a Cas9 protein, a nucleic acid encoding a first guide RNA, and a nucleic acid that is the reverse complement to the coding sequence for the second guide RNA. In some embodiments, the payload comprises a single nucleic acid molecule, wherein the single nucleic acid molecule comprises a nucleic acid encoding a Cas9 protein, a nucleic acid that is the reverse complement to the coding sequence for the first guide RNA, and a nucleic acid that is the reverse complement to the coding sequence for the second guide RNA. In some embodiments, the nucleic acid encoding a Cas9 protein (e.g., an SaCas9 or SluCas9 protein) is under the control of the CK8e promoter. In some embodiments, the first guide is under the control of the 7SK2 promoter, and the second guide is under the control of the H1m promoter. In some embodiments, the first guide is under the control of the H1m promoter, and the second guide is under the control of the 7SK2 promoter. In some embodiments, the first guide is under the control of the hU6c promoter, and the second guide is under the control of the H1m promoter. In some embodiments, the first guide is under the control of the H1m promoter, and the second guide is under the control of the hU6c promoter. In some embodiments, the nucleic acid encoding the Cas9 protein is: a) between the nucleic acids encoding the guide RNAs, b) between the nucleic acids that are the reverse complement to the coding sequences for the guide RNAs, c) between the nucleic acid encoding the first guide RNA and the nucleic acid that is the reverse complement to the coding sequence for the second guide RNA, d) between the nucleic acid encoding the second guide RNA and the nucleic acid that is the reverse complement to the coding sequence for the first guide RNA, e) 5′ to the nucleic acids encoding the guide RNAs, f) 5′ to the nucleic acids that are the reverse complements to the coding sequences for the guide RNAs, g) 5′ to a nucleic acid encoding one of the guide RNAs and 5′ to a nucleic acid that is the reverse complement to the coding sequence for the other guide RNA, h) 3′ to the nucleic acids encoding the guide RNAs, i) 3′ to the nucleic acids that are the reverse complements to the coding sequences for the guide RNAs, or j) 3′ to a nucleic acid encoding one of the guide RNAs and 3′ to a nucleic acid that is the reverse complement to the coding sequence for the other guide RNA.

In some embodiments, the payload comprises a nucleic acid encoding at least a first guide RNA and a second guide RNA. In some embodiments, the nucleic acid comprises a spacer-encoding sequence for the first guide RNA, a scaffold-encoding sequence for the first guide RNA, a spacer-encoding sequence for the second guide RNA, and a scaffold-encoding sequence of the second guide RNA. In some embodiments, the spacer-encoding sequence (e.g., encoding any of the spacer sequences disclosed herein) for the first guide RNA is identical to the spacer-encoding sequence for the second guide RNA. In some embodiments, the spacer-encoding sequence (e.g., encoding any of the spacer sequences disclosed herein) for the first guide RNA is different from the spacer-encoding sequence for the second guide RNA. In some embodiments, the scaffold-encoding sequence for the first guide RNA is identical to the scaffold-encoding sequence for the second guide RNA. In some embodiments, the scaffold-encoding sequence for the first guide RNA is different from the scaffold-encoding sequence for the nucleic acid encoding the second guide RNA.

In some embodiments, the payload comprises from 5′ to 3′ with respect to the plus strand: the reverse complement of a first sgRNA scaffold sequence, the reverse complement of a nucleic acid encoding a first sgRNA guide sequence, the reverse complement of a promoter for expression of the nucleic acid encoding the first sgRNA, a promoter for expression of a nucleic acid encoding SaCas9 (e.g., CK8e), a nucleic acid encoding SaCas9, a polyadenylation sequence, a promoter for expression of a second sgRNA, a second sgRNA guide sequence, and a second sgRNA scaffold sequence. In some embodiments the promoter for expression of the nucleic acid encoding the first and/or second sgRNA is a hU6c promoter or a 7SK2 promoter. In some embodiments the promoter for expression of the nucleic acid encoding the second sgRNA is a H1m promoter. In some embodiments, the promoter for SaCas9 is the CK8e promoter. In some embodiments, the nucleic acid sequence encoding SaCas9 is fused to a nucleic acid sequence encoding a nuclear localization sequence (NLS). In some embodiments, the nucleic acid sequence encoding SaCas9 is fused to two nucleic acid sequences each encoding a nuclear localization sequence (NLS). In some embodiments, the nucleic acid sequence encoding SaCas9 is fused to three nucleic acid sequences each encoding a nuclear localization sequence (NLS). In some embodiments, the one or more NLSs is an SV40 NLS. In some embodiments, the one or more NLSs is a c-Myc NLS. In some embodiments, the NLS is fused to the SaCas9 with a linker.

In some embodiments, the payload is directed to a liver and non-liver target. In some embodiments, the non-liver target is the muscle. In such embodiments, the non-liver payload-based gene therapy is used to treat DMD.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 320 (designated herein as SpCas9):

MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVDE
VAYHEKYPTIYHLRKKLVDSTDKADLRLIYLALAHMIKFRGHFLIEGDLNPDNSDVDKLFIQL
VQTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNE
KVLPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVKVMGRH
KPENIVIEMARENQTTQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQILDSRM
NTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKKDW
DPKKYGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGD.

In some embodiments, the nucleic acid encoding SaCas9 encodes an SaCas9 comprising an amino acid sequence that is at least 80%, 85%, 90%, 91%, 92%, 93%, 94%, 95%, 96%, 97%, 98%, 99% or 100% identical to the sequence of SEQ ID NO: 306:

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRELINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYNNDLIKINGELYRVIGVNNDLLNRIEVNMIDITYREYLENMN
DKRPPRIIKTIASKTQSIKKYSTDILGNLYEVKSKKHPQIIKKG.

In some embodiments, the SaCas9 comprises an amino acid sequence that is at least 80%, 85%, 90%, 91%, 92%, 93%, 94%, 95%, 96%, 97%, 98%, 99% or 100% identical to the sequence of SEQ ID NO: 307 (designated herein as SaCas9-KKH or SACAS9KKH):

KRNYILGLDIGITSVGYGIIDYETRDVIDAGVRLFKEANVENNEGRRSKRGARRLKRRRRHRI
QRVKKLLFDYNLLTDHSELSGINPYEARVKGLSQKLSEEEFSAALLHLAKRRGVHNVNEVEE
DTGNELSTKEQISRNSKALEEKYVAELQLERLKKDGEVRGSINRFKTSDYVKEAKQLLKVQK
AYHQLDQSFIDTYIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIAKEILVNEEDIKGY
RVTSTGKPEFTNLKVYHDIKDITARKEIIENAELLDQIAKILTIYQSSEDIQEELTNLNSELTQEE
IEQISNLKGYTGTHNLSLKAINLILDELWHTNDNQIAIFNRLKLVPKKVDLSQQKEIPTTLVDD
FILSPVVKRSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSFNNKVL
VKQEENSKKGNRTPFQYLSSSDSKISYETFKKHILNLAKGKGRISKTKKEYLLEERDINRFSVQ
KDFINRNLVDTRYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQEYKEIFITPHQIKHI
KDFKDYKYSHRVDKKPNRKLINDTLYSTRKDDKGNTLIVNNLNGLYDKDNDKLKKLINKSP
EKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKC
YEEAKKLKKISNQAEFIASFYKNDLIKINGELYRVIGVNNDLLNRIEVNMIDITYREYLENMN
DKRPPHIIKTIASKTQSIKKYSTDILGNLYEVKSKKHPQIIKKG.

In some embodiments, the nucleic acid encoding SluCas9 encodes a SluCas9 comprising an amino acid sequence that is at least 80%, 85%, 90%, 91%, 92%, 93%, 94%, 95%, 96%, 97%, 98%, 99% or 100% identical to the sequence of SEQ ID NO: 308:

NQKFILGLDIGITSVGYGLIDYETKNIIDAGVRLFPEANVENNEGRRSKRGSRRLKRRRIHRLE
RVKKLLEDYNLLDQSQIPQSTNPYAIRVKGLSEALSKDELVIALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGKGSPYGWEGDPKAWYETLMGHCTYFPDELRSVKYAY
SADLFNALNDLNNLVIQRDGLSKLEYHEKYHIIENVFKQKKKPTLKQIANEINVNPEDIKGYRI
TKSGKPQFTEFKLYHDLKSVLFDQSILENEDVLDQIAEILTIYQDKDSIKSKLTELDILLNEEDK
ENIAQLTGYTGTHRLSLKCIRLVLEEQWYSSRNQMEIFTHLNIKPKKINLTAANKIPKAMIDEF
ILSPVVKRTFGQAINLINKIIEKYGVPEDIIIELARENNSKDKQKFINEMQKKNENTRKRINEIIG
KYGNQNAKRLVEKIRLHDEQEGKCLYSLESIPLEDLLNNPNHYEVDHIIPRSVSFDNSYHNKV
LVKQSENSKKSNLTPYQYFNSGKSKLSYNQFKQHILNLSKSQDRISKKKKEYLLEERDINKFE
VQKEFINRNLVDTRYATRELTNYLKAYFSANNMNVKVKTINGSFTDYLRKVWKFKKERNH
GYKHHAEDALIIANADFLFKENKKLKAVNSVLEKPEIETKQLDIQVDSEDNYSEMFIIPKQVQ
DIKDFRNFKYSHRVDKKPNRQLINDTLYSTRKKDNSTYIVQTIKDIYAKDNTTLKKQFDKSPE
KFLMYQHDPRTFEKLEVIMKQYANEKNPLAKYHEETGEYLTKYSKKNNGPIVKSLKYIGNK
LGSHLDVTHQFKSSTKKLVKLSIKPYRFDVYLTDKGYKFITISYLDVLKKDNYYYIPEQKYDK
LKLGKAIDKNAKFIASFYKNDLIKLDGEIYKIIGVNSDTRNMIELDLPDIRYKEYCELNNIKGEP
RIKKTIGKKVNSIEKLTTDVLGNVFTNTQYTKPQLLFKRGN.

In some embodiments, the Cas protein is any of the engineered Cas proteins disclosed in Schmidt et al., 2021, Nature Communications, “Improved CRISPR genome editing using small highly active and specific engineered RNA-guided nucleases.”

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 309 (designated herein as sRGN1):

MNQKFILGLDIGITSVGYGLIDYETKNIIDAGVRLFPEANVENNEGRRSKRGSRRLKRRRIHRL
DRVKHLLAEYDLLDLTNIPKSTNPYQTRVKGLNEKLSKDELVIALLHIAKRRGIHNVDVAAD
KEETASDSLSTKDQINKNAKFLESRYVCELQKERLENEGHVRGVENRFLTKDIVREAKKIIDT
QMQYYPEIDETFKEKYISLVETRREYFEGPGKGSPFGWEGNIKKWFEQMMGHCTYFPEELRS
VKYSYSAELFNALNDLNNLVITRDEDAKLNYGEKFQIIENVFKQKKTPNLKQIAIEIGVHETEI
KGYRVNKSGTPEFTEFKLYHDLKSIVFDKSILENEAILDQIAEILTIYQDEQSIKEELNKLPEILN
EQDKAEIAKLIGYNGTHRLSLKCIHLINEELWQTSRNQMEIFNYLNIKPNKVDLSEQNKIPKD
MVNDFILSPVVKRTFIQSINVINKVIEKYGIPEDIIIELARENNSDDRKKFINNLQKKNEATRKRI
NEIIGQTGNQNAKRIVEKIRLHDQQEGKCLYSLKDIPLEDLLRNPNNYDIDHIIPRSVSFDDSM
HNKVLVRREQNAKKNNQTPYQYLTSGYADIKYSVFKQHVLNLAENKDRMTKKKREYLLEE
RDINKFEVQKEFINRNLVDTRYATRELTNYLKAYFSANNMNVKVKTINGSFTDYLRKVWKF
KKERNHGYKHHAEDALIIANADFLFKENKKLKAVNSVLEKPEIETKQLDIQVDSEDNYSEMFI
IPKQVQDIKDFRNFKYSHRVDKKPNRQLINDTLYSTRKKDNSTYIVQTIKDIYAKDNTTLKKQ
FDKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 310 (designated herein as sRGN2):

MNQKFILGLDIGITSVGYGLIDYETKNIIDAGVRLFPEANVENNEGRRSKRGSRRLKRRRIHRL
ERVKSLLSEYKIISGLAPTNNQPYNIRVKGLTEQLTKDELAVALLHIAKRRGIHKIDVIDSNDD
VGNELSTKEQLNKNSKLLKDKFVCQIQLERMNEGQVRGEKNRFKTADIIKEIIQLLNVQKNFH
QLDENFINKYIELVEMRREYFEGPGQGSPFGWNGDLKKWYEMLMGHCTYFPQELRSVKYA
YSADLFNALNDLNNLIIQRDNSEKLEYHEKYHIIENVFKQKKKPTLKQIAKEIGVNPEDIKGYR
ITKSGTPEFTEFKLYHDLKSVLFDQSILENEDVLDQIAEILTIYQDKDSIKSKLTELDILLNEEDK
ENIAQLTGYNGTHRLSLKCIRLVLEEQWYSSRNQMEIFTHLNIKPKKINLTAANKIPKAMIDEF
ILSPVVKRTFIQSINVINKVIEKYGIPEDIIIELARENNSDDRKKFINNLQKKNEATRKRINEIIGQ
TGNQNAKRIVEKIRLHDQQEGKCLYSLESIALMDLLNNPQNYEVDHIIPRSVAFDNSIHNKVL
VKQIENSKKGNRTPYQYLNSSDAKLSYNQFKQHILNLSKSKDRISKKKKDYLLEERDINKFEV
QKEFINRNLVDTRYATRELTSYLKAYFSANNMDVKVKTINGSFTNHLRKVWRFDKYRNHGY
KHHAEDALIIANADFLFKENKKLKAVNSVLEKPEIETKQLDIQVDSEDNYSEMFIIPKQVQDIK
DFRNFKYSHRVDKKPNRQLINDTLYSTRKKDNSTYIVQTIKDIYAKDNTTLKKQFDKSPEKFL
MYQHDPRTFEKLEVIMKQYANEKNPLAKYHEETGEYLTKYSKKNNGPIVKSLKYIGNKLGS
HLDVTHQFKSSTKKLVKLSIKPYRFDVYLTDKGYKFITISYLDVLKKDNYYYIPEQKYDKLKL
GKAIDKNAKFIASFYKNDLIKLDGEIYKIIGVNSDTRNMIELDLPDIRYKEYCELNNIKGEPRIK
KTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 311 (designated herein as sRGN3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKPYRFDVYLTDKGYKFITISYLDVLKKDNYYYIPE
QKYDKLKLGKAIDKNAKFIASFYKNDLIKLDGEIYKIIGVNSDTRNMIELDLPDIRYKEYCELN
NIKGEPRIKKTIGKKVNSIEKLTTDVLGNVFTNTQYTKPQLLFKRGN.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 312 (designated herein as sRGN3.1):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKYSHRVDKKPNRQLINDTLYSTRKKDNSTYIVQTIKDIYAKDNTTLKKQF
DKSPEKFLMYQHDPRTFEKLEVIMKQYANEKNPLAKYHEETGEYLTKYSKKNNGPIVKSLK
YIGNKLGSHLDVTHQFKSSTKKLVKLSIKNYRFDVYLTEKGYKFVTIAYLNVFKKDNYYYIP
KDKYQELKEKKKIKDTDQFIASFYKNDLIKLNGDLYKIIGVNSDDRNIIELDYYDIKYKDYCEI
NNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 313 (designated herein as sRGN3.2):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTNYLKAYFSANNMNVKVKTINGSFTDYLRKVWKFK
KERNHGYKHHAEDALIIANADFLFKENKKLKAVNSVLEKPEIETKQLDIQVDSEDNYSEMFII
PKQVQDIKDFRNFKFSHRVDKKPNRQLINDTLYSTRMKDEHDYIVQTITDIYGKDNTNLKKQ
FNKNPEKFLMYQNDPKTFEKLSIIMKQYSDEKNPLAKYYEETGEYLTKYSKKNNGPIVKKIK
LLGNKVGNHLDVTNKYENSTKKLVKLSIKNYRFDVYLTEKGYKFVTIAYLNVFKKDNYYYI
PKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 314 (designated herein as sRGN3.3):

MNQKFILGLDIGITSVGYGLIDYETKNIIDAGVRLFPEANVENNEGRRSKRGSRRLKRRRIHRL
ERVKLLLTEYDLINKEQIPTSNNPYQIRVKGLSEILSKDELAIALLHLAKRRGIHNVDVAADKE
ETASDSLSTKDQINKNAKFLESRYVCELQKERLENEGHVRGVENRFLTKDIVREAKKIIDTQM
QYYPEIDETFKEKYISLVETRREYFEGPGQGSPFGWNGDLKKWYEMLMGHCTYFPQELRSV
KYAYSADLFNALNDLNNLIIQRDNSEKLEYHEKYHIIENVFKQKKKPTLKQIAKEIGVNPEDIK
GYRITKSGTPEFTSFKLFHDLKKVVKDHAILDDIDLLNQIAEILTIYQDKDSIVAELGQLEYLM
SEADKQSISELTGYTGTHSLSLKCMNMIIDELWHSSMNQMEVFTYLNMRPKKYELKGYQRIP
TDMIDDAILSPVVKRTFIQSINVINKVIEKYGIPEDIIIELARENNSDDRKKFINNLQKKNEATRK
RINEIIGQTGNQNAKRIVEKIRLHDQQEGKCLYSLESIPLEDLLNNPNHYEVDHIIPRSVSFDNS
YHNKVLVKQSENSKKSNLTPYQYFNSGKSKLSYNQFKQHILNLSKSQDRISKKKKEYLLEER
DINKFEVQKEFINRNLVDTRYATRELTSYLKAYFSANNMDVKVKTINGSFTNHLRKVWRFD
KYRNHGYKHHAEDALIIANADFLFKENKKLQNTNKILEKPTIENNTKKVTVEKEEDYNNVFE
TPKLVEDIKQYRDYKFSHRVDKKPNRQLINDTLYSTRMKDEHDYIVQTITDIYGKDNTNLKK
QFNKNPEKFLMYQNDPKTFEKLSIIMKQYSDEKNPLAKYYEETGEYLTKYSKKNNGPIVKKI
KLLGNKVGNHLDVTNKYENSTKKLVKLSIKNYRFDVYLTEKGYKFVTIAYLNVFKKDNYYY
IPKDKYQELKEKKKIKDTDQFIASFYKNDLIKLNGDLYKIIGVNSDDRNIIELDYYDIKYKDYC
EINNIKGEPRIKKTIGKKTESIEKFTTDVLGNLYLHSTEKAPQLIFKRGL.

In some embodiments, the Cas9 comprises an amino acid sequence that is at least 80%, 85%, 90%, 91%, 92%, 93%, 94%, 95%, 96%, 97%, 98%, 99% or 100% identical to the sequence of SEQ ID NO: 315 (designated herein as sRGN4):

MNQKFILGLDIGITSVGYGLIDYETKNIIDAGVRLFPEANVENNEGRRSKRGSRRLKRRRIHRL
ERVKKLLEDYNLLDQSQIPQSTNPYAIRVKGLSEALSKDELVIALLHIAKRRGIHNINVSSEDE
DASNELSTKEQINRNNKLLKDKYVCEVQLQRLKEGQIRGEKNRFKTTDILKEIDQLLKVQKD
YHNLDIDFINQYKEIVETRREYFEGPGKGSPYGWEGDPKAWYETLMGHCTYFPDELRSVKY
AYSADLFNALNDLNNLVIQRDGLSKLEYHEKYHIIENVFKQKKKPTLKQIANEINVNPEDIKG
YRITKSGKPEFTSFKLFHDLKKVVKDHAILDDIDLLNQIAEILTIYQDKDSIVAELGQLEYLMS
EADKQSISELTGYTGTHSLSLKCMNMIIDELWHSSMNQMEVFTYLNMRPKKYELKGYQRIPT
DMIDDAILSPVVKRTFIQSINVINKVIEKYGIPEDIIIELARENNSDDRKKFINNLQKKNEATRKR
INEIIGQTGNQNAKRIVEKIRLHDQQEGKCLYSLESIPLEDLLNNPNHYEVDHIIPRSVSFDNSY
HNKVLVKQSENSKKSNLTPYQYFNSGKSKLSYNQFKQHILNLSKSQDRISKKKKEYLLEERDI
NKFEVQKEFINRNLVDTRYATRELTNYLKAYFSANNMNVKVKTINGSFTDYLRKVWKFKKE
RNHGYKHHAEDALIIANADFLFKENKKLKAVNSVLEKPEIETKQLDIQVDSEDNYSEMFIIPK
QVQDIKDFRNFKYSHRVDKKPNRQLINDTLYSTRKKDNSTYIVQTIKDIYAKDNTTLKKQFD
KSPEKFLMYQHDPRTFEKLEVIMKQYANEKNPLAKYHEETGEYLTKYSKKNNGPIVKSLKYI
GNKLGSHLDVTHQFKSSTKKLVKLSIKPYRFDVYLTDKGYKFITISYLDVLKKDNYYYIPEQK
YDKLKLGKAIDKNAKFIASFYKNDLIKLDGEIYKIIGVNSDTRNMIELDLPDIRYKEYCELNNI
KGEPRIKKTIGKKVNSIEKLTTDVLGNVFTNTQYTKPQLLFKRGN.

In some embodiments, the payload comprises a single-molecule guide RNA (sgRNA). An sgRNA can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and/or an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins. In particular embodiments, the disclosure provides for an sgRNA comprising a spacer sequence and a tracrRNA sequence.

An exemplary scaffold sequence suitable for use with SaCas9 to follow the guide sequence at its 3′ end is: GTTTAAGTACTCTGTGCTGGAAACAGCACAGAATCTACTTAAACAAGGCAAAATGCCGT GTTTATCTCGTCAACTTGTTGGCGAGA (SEQ ID NO: 500) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 500, or a sequence that differs from SEQ ID NO: 500 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV1” and is: GTTTTAGTACTCTGGAAACAGAATCTACTAAAACAAGGCAAAATGCCGTGTTTATCTCGT CAACTTGTTGGCGAGAT (SEQ ID NO: 501) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 910, or a sequence that differs from SEQ ID NO: 910 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV2” and is: GTTTAAGTACTCTGTGCTGGAAACAGCACAGAATCTACTTAAACAAGGCAAAATGCCGT GTTTATCTCGTCAACTTGTTGGCGAGAT (SEQ ID NO: 502) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 911, or a sequence that differs from SEQ ID NO: 911 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV3” and is: GTTTAAGTACTCTGGAAACAGAATCTACTTAAACAAGGCAAAATGCCGTGTTTATCTCGT CAACTTGTTGGCGAGAT (SEQ ID NO: 503) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 912, or a sequence that differs from SEQ ID NO: 912 by no more than 1, 2, 3, 4, 5, 10, 15, 20, or 25 nucleotides.

In some embodiments, a variant of an SaCas9 scaffold sequence may be used. In some embodiments, the SaCas9 scaffold to follow the guide sequence at its 3′ end is referred to as “SaScaffoldV5” and is: GTTTCAGTACTCTGGAAACAGAATCTACTGAAACAAGGCAAAATGCCGTGTTTATCTCGT CAACTTGTTGGCGAGAT (SEQ ID NO: 932) in 5′ to 3′ orientation. In some embodiments, an exemplary scaffold sequence for use with SaCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 932, or a sequence that differs from SEQ ID NO: 932 by no more than 1, 2, 3, 4, 5, 10, 15, 20, or 25 nucleotides.

Two exemplary scaffold sequences suitable for use with SluCas9 to follow the guide sequence at its 3′ end is: GTTTTAGTACTCTGGAAACAGAATCTACTGAAACAAGACAATATGTCGTGTTTATCCCAT CAATTTATTGGTGGGAT (SEQ ID NO: 900), and GTTTAAGTACTCTGTGCTGGAAACAGCACAGAATCTACTGAAACAAGACAATATGTCGT GTTTATCCCATCAATTTATTGGTGGGA (SEQ ID NO: 601) in 5′ to 3′ orientation. In some embodiments, an exemplary sequence for use with SluCas9 to follow the 3′ end of the guide sequence is a sequence that is at least 80%, 85%, 90%, 91%, 92%, 93%, 94%, 95%, 96%, 97%, 98%, 99% or 100% identical to SEQ ID NO: 900 or SEQ ID NO: 601, or a sequence that differs from SEQ ID NO: 900 or SEQ ID NO: 601 by no more than 1, 2, 3, 4, 5, 10, 15, 20, or 25 nucleotides.

Exemplary scaffold sequences suitable for use with SluCas9 to follow the guide sequence at its 3′ end are also shown below in the 5′ to 3′ orientation:

| | | | | Streak of |
|---|---|---|---|---|
| | | | | Homology to |
| Scaffold | SEQ | | Homology | Slu v5 |
| ID | ID NO | Scaffold Sequence (5′ to 3′) | to Slu v5 | (# nucleotides) |
| Wildtype | 900 | GTTTTAGTACTCTGGAAACAGAATCTACTGAA | N/A | N/A |
| | | ACAAGACAATATGTCGTGTTTATCCCATCAAT | | |
| | | TTATTGGTGGGAT | | |
| Slu- | 601 | GTTTAAGTACTCTGTGCTGGAAACAGCACAG | N/A | N/A |
| VCGT- | | AATCTACTGAAACAAGACAATATGTCGTGTTT | | |
| 4.5 | | ATCCCATCAATTTATTGGTGGGA | | |
| Slu_v5 | 901 | GTTTCAGTACTCTGGAAACAGAATCTACTGAA | 100.00% | 77 |
| | | ACAAGACAATATGTCGTGTTTATCCCATCAAT | | |
| | | TTATTGGTGGGAT | | |
| Slu_v5-1 | 902 | GTTTggTaACcTaGGAAACTagATCTTaccAAACA | 87.50% | 47 |
| | | AGACAATATGTCGTGTTTATCCCATCAATTTA | | |
| | | TTGGTGGGAT | | |
| Slu_v5-2 | 903 | GTTTCAGTACTCTGGAAACAGAATCTACTGAA | 96.10% | 37 |
| | | ACAAGgCAAaATGcCGTGTTTATCCCATCAATT | | |
| | | TATTGGTGGGAT | | |
| Slu_v5-3 | 904 | GTTTCAGTACTCTGGAAACAGAATCTACTGAA | 94.81% | 48 |
| | | ACAAGACAATATGTCGcgcccaTCCCATCAATTT | | |
| | | ATTGGTGGGAT | | |
| Slu_v5-4 | 905 | GTTTCAGTACTCTGGAAACAGAATCTACTGAA | 91.55% | 55 |
| | | ACAAGACAATATGTCGTGTTTATgggTTgAATT | | |
| | | TATTcGacccAT | | |
| Slu_v5-5 | 906 | GTTTggTaACcTaGGAAACTagATCTTaccAAACA | 83.75% | 31 |
| | | AGgCAAaATGcCGTGTTTATCCCATCAATTTAT | | |
| | | TGGTGGGAT | | |
| Slu_v5-6 | 907 | GTTTggTaACcTaGGAAACTagATCTTaccAAACA | 82.50% | 23 |
| | | AGACAATATGTCGcgcccaTCCCATCAATTTATT | | |
| | | GGTGGGAT | | |
| Slu_v5-7 | 908 | GTTTggTaACcTaGGAAACTagATCTTaccAAACA | 78.38% | 25 |
| | | AGACAATATGTCGTGTTTATgggTTgAATTTAT | | |
| | | TcGacccAT | | |
| Slu_v5-8 | 909 | GTTTCAGTACTCTGGAAACAGAATCTACTGAA | 90.91% | 37 |
| | | ACAAGgCAAaATGcCGcgcccaTCCCATCAATTTA | | |
| | | TTGGTGGGAT | | |
| Slu_v5-9 | 910 | GTTTCAGTACTCTGGAAACAGAATCTACTGAA | 87.32% | 37 |
| | | ACAAGgCAAaATGcCGTGTTTATgggTTgAATTT | | |
| | | ATTcGacccAT | | |
| Slu_v5- | 911 | GTTTCAGTACTCTGGAAACAGAATCTACTGAA | 82.89% | 48 |
| 10 | | ACAAGACAATATGTCGcgcccaTgggTTgAATTTA | | |
| | | TTcGacccAT | | |
| Slu_v5- | 912 | GTTTggTaACcTaGGAAACTagATCTTaccAAACA | 78.75% | 23 |
| 11 | | AGgCAAaATGcCGcgcccaTCCCATCAATTTATTG | | |
| | | GTGGGAT | | |
| Slu_v5- | 913 | GTTTggTaACcTaGGAAACTagATCTTaccAAACA | 74.32% | 9 |
| 12 | | AGgCAAaATGcCGTGTTTATgggTTgAATTTATTc | | |
| | | GacccAT | | |
| Slu_v5- | 914 | GTTTggTaACcTaGGAAACTagATCTTaccAAACA | 70.89% | 18 |
| 13 | | AGACAATATGTCGcgcccaTgggTTgAATTTATTc | | |
| | | GacccAT | | |
| Slu_v5- | 915 | GTTTCAGTACTCTGGAAACAGAATCTACTGAA | 78.95% | 37 |
| 14 | | ACAAGgCAAaATGcCGcgcccaTgggTTgAATTTAT | | |
| | | TcGacccAT | | |
| Slu_v5- | 916 | GTTTggTaACcTaGGAAACTagATCTTaccAAACA | 67.09% | 8 |
| 15 | | AGgCAAaATGcCGcgcccaTgggTTgAATTTATTcGa | | |
| | | cccAT | | |
| Slu_v4 | 917 | GTTTCAGTACTCTGTGCTGGAAACAGCACAGA | N/A | N/A |
| | | ATCTACTGAAACAAGACAATATGTCGTGTTTA | | |
| | | TCCCATCAATTTATTGGTGGGAT | | |

In some embodiments, the scaffold sequence suitable for use with SluCas9 to follow the guide sequence at its 3′ end is selected from any one of SEQ ID NOs: 900 or 601, or 901-917 in 5′ to 3 orientation (see below). In some embodiments, an exemplary sequence for use with SluCas9 to follow the 3′ end of the guide sequence is a sequence that is at least 65% o, 70%, 75%, 80%, 85% o, 90%, 91%, 92% o, 93%, 94%, 95%, 96% o, 97%, 98% o, 99% o or 100% o identical to any one off SEQ ID NOs: 900 or 601, or 901-917, or a sequence that differs from any one of SEQ ID NOs: 900 or 601, or 901-917 by no more than 1, 2, 3, 4, 5, 10, 15, 20, or 25 nucleotides.

In some embodiments, the scaffold sequence suitable for use with SluCas9 to follow the guide sequence at its 3′ end is selected from any one of SEQ ID NOs: 901-917 in 5′ to 3 orientation (see below). In some embodiments, an exemplary sequence for use with SluCas9 to follow the 3′ end of the guide sequence is a sequence that is at least 65%, 70%, 75%, 80%, 85%, 90%, 91%, 92%, 93%, 94%, 95%, 96%, 97%, 98%, 99% or 100% identical to any one off SEQ ID NOs: 901-917, or a sequence that differs from any one of SEQ ID NOs: 901-917 by no more than 1, 2, 3, 4, 5, 10, 15, 20, or 25 nucleotides.

In some embodiments, the nucleic acid encoding the gRNA or the nucleic acid encoding the pair of gRNAs comprises a sequence comprising SEQ ID NO: 900. In some embodiments, the nucleic acid encoding the gRNA or the nucleic acid encoding the pair of gRNAs comprises a sequence comprising SEQ ID NO: 601. In some embodiments, the nucleic acid encoding the gRNA or the nucleic acid encoding the pair of gRNAs comprises a sequence comprising SEQ ID NO: 900. In some embodiments, the nucleic acid encoding the gRNA or the nucleic acid encoding the pair of gRNAs comprises a sequence comprising SEQ ID NO: 901. In some embodiments, the nucleic acid encoding the gRNA or the nucleic acid encoding the pair of gRNAs comprises a sequence comprising SEQ ID NO: 902. In some embodiments, the nucleic acid encoding the gRNA or the nucleic acid encoding the pair of gRNAs comprises a sequence comprising SEQ ID NO: 903. In some embodiments, the nucleic acid encoding the gRNA or the nucleic acid encoding the pair of gRNAs comprises a sequence comprising SEQ ID NO: 904. In some embodiments, the nucleic acid encoding the gRNA or the nucleic acid encoding the pair of gRNAs comprises a sequence comprising SEQ ID NO: 905. In some embodiments, the nucleic acid encoding the gRNA or the nucleic acid encoding the pair of gRNAs comprises a sequence comprising SEQ ID NO: 906. In some embodiments, the nucleic acid encoding the gRNA or the nucleic acid encoding the pair of gRNAs comprises a sequence comprising SEQ ID NO: 907. In some embodiments, the nucleic acid encoding the gRNA or the nucleic acid encoding the pair of gRNAs comprises a sequence comprising SEQ ID NO: 908. In some embodiments, the nucleic acid encoding the gRNA or the nucleic acid encoding the pair of gRNAs comprises a sequence comprising SEQ ID NO: 909. In some embodiments, the nucleic acid encoding the gRNA or the nucleic acid encoding the pair of gRNAs comprises a sequence comprising SEQ ID NO: 910. In some embodiments, the nucleic acid encoding the gRNA or the nucleic acid encoding the pair of gRNAs comprises a sequence comprising SEQ ID NO: 911. In some embodiments, the nucleic acid encoding the gRNA or the nucleic acid encoding the pair of gRNAs comprises a sequence comprising SEQ ID NO: 912. In some embodiments, the nucleic acid encoding the gRNA or the nucleic acid encoding the pair of gRNAs comprises a sequence comprising SEQ ID NO: 913. In some embodiments, the nucleic acid encoding the gRNA or the nucleic acid encoding the pair of gRNAs comprises a sequence comprising SEQ ID NO: 914. In some embodiments, the nucleic acid encoding the gRNA or the nucleic acid encoding the pair of gRNAs comprises a sequence comprising SEQ ID NO: 915. In some embodiments, the nucleic acid encoding the gRNA or the nucleic acid encoding the pair of gRNAs comprises a sequence comprising SEQ ID NO: 916. In some embodiments, the nucleic acid encoding the gRNA or the nucleic acid encoding the pair of gRNAs comprises a sequence comprising SEQ ID NO: 917. In some embodiments, comprising a pair of gRNAs, one of the gRNAs comprises a sequence selected from any one of SEQ ID NOs: 900 or 601, or 901-917. In some embodiments, comprising a pair of gRNAs, both of the gRNAs comprise a sequence selected from any one of SEQ ID NOs: 900 or 601, or 901-917. In some embodiments, comprising a pair of gRNAs, the nucleotides 3′ of the guide sequence of the gRNAs are the same sequence. In some embodiments, comprising a pair of gRNAs, the nucleotides 3′ of the guide sequence of the gRNAs are different sequences.

In some embodiments, the scaffold sequence comprises one or more alterations in the stem loop 1 as compared to the stem loop 1 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the stem loop 2 as compared to the stem loop 2 of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the tetraloop as compared to the tetraloop of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the repeat region as compared to the repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the anti-repeat region as compared to the anti-repeat region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). In some embodiments, the scaffold sequence comprises one or more alterations in the linker region as compared to the linker region of a wildtype SluCas9 scaffold sequence (e.g., a scaffold comprising the sequence of SEQ ID NO: 900) or a reference SluCas9 scaffold sequence (e.g., a scaffold comprising the sequence of SEQ ID NO: 901). See, e.g., Nishimasu et al., 2015, Cell, 162:1113-1126 for description of regions of a scaffold.

Where a tracrRNA is used, in some embodiments, it comprises (5′ to 3′) a second complementary domain and a proximal domain. In the case of a sgRNA, guide sequences together with additional nucleotides (e.g., SEQ ID Nos: 500, or 910-912 (for SaCas9), and 900 or 601, or 901-917 (for SluCas9)) form or encode a sgRNA. In some embodiments, an sgRNA comprises (5′ to 3′) at least a spacer sequence, a first complementary domain, a linking domain, a second complementary domain, and a proximal domain. A sgRNA or tracrRNA may further comprise a tail domain. The linking domain may be hairpin-forming. See, e.g., US 2017/0007679 for detailed discussion and examples of crRNA and gRNA domains, including second complementarity domains, linking domains, proximal domains, and tail domains.

In some embodiments, the payload comprises a nucleic acid molecule comprising at least two guide RNAs, wherein once expressed in vivo or in vivo, the guide RNAs excise a portion of the exon, wherein the size of excised portion of the exon is between 8 and 167 nucleotides.

In some embodiments, the guide RNAs comprise as non-limiting examples the guide sequences disclosed in Tables 1A, 1B, and Table 2 below. For example, when the payload comprises SaCas9, one or more guide sequences is selected from any one of SEQ ID NOs: 1-35, 1000-1078, and 3000-3069; or when the payload comprises SluCas9, one or more guide sequences selected from any one of SEQ ID NOs: 100-225, 2000-2116, and 4000-4251 from the tables below. Additional exemplary payload compositions, including varieties of RNP complexes (comprising one or more guide RNAs comprising and saCas9 or sluCas9, or a mutant Cas9 protein), are disclosed elsewhere in WO2022/056000, which is incorporated herein in its entirety.

In some embodiments, the payload comprises a nucleic acid molecule encoding one or more guide RNAs and a Cas9, wherein the nucleic acid molecule comprises a first nucleic acid encoding one or more guide RNAs selected from any one of SEQ ID NOs: 1-35, 1000-1078, or 3000-3069 and a second nucleic acid encoding a Staphylococcus aureus Cas9 (SaCas9).

In some embodiments, the payload comprises a nucleic acid molecule encoding one or more guide RNAs and a Cas9, wherein the nucleic acid molecule comprises a first nucleic acid encoding one or more guide RNAs selected from any one of SEQ ID NOs: 100-225, 2000-2116, or 4000-4251 and a second nucleic acid encoding a Staphylococcus lugdunensis (SluCas9).

In some embodiments, the payload comprises a nucleic acid molecule encoding one or more guide RNAs and a Cas9, wherein the nucleic acid molecule comprises a first nucleic acid encoding one or more guide RNAs comprising at least 20 contiguous nucleotides of a guide RNA selected from any one of SEQ ID NOs: 1-35, 1000-1078, or 3000-3069 and a second nucleic acid encoding a Staphylococcus aureus Cas9 (SaCas9).

In some embodiments, the payload comprises a nucleic acid molecule encoding one or more guide RNAs and a Cas9, wherein the nucleic acid molecule comprises a first nucleic acid encoding one or more guide RNAs comprising at least 20 contiguous nucleotides of a guide RNA selected from any one of SEQ ID NOs: 100-225, 2000-2116, or 4000-4251 and a second nucleic acid encoding a Staphylococcus lugdunensis (SluCas9).

In some embodiments, the payload comprises a nucleic acid molecule encoding one or more guide RNAs and a Cas9, wherein the nucleic acid molecule comprises a first nucleic acid encoding one or more guide RNAs that is at least 90% identical to any one of SEQ ID NOs: 1-35, 1000-1078, or 3000-3069 and a second nucleic acid encoding a Staphylococcus aureus Cas9 (SaCas9).

In some embodiments, the payload comprises a nucleic acid molecule encoding one or more guide RNAs and a Cas9, wherein the nucleic acid molecule comprises a first nucleic acid encoding one or more guide RNAs that is at least 90% identical to any one of SEQ ID NOs: 100-225, 2000-2116, or 4000-4251 and a second nucleic acid encoding a Staphylococcus lugdunensis (SluCas9).

In some embodiments, the payload comprises a nucleic acid molecule encoding one or more guide RNAs and a Cas9, wherein the nucleic acid molecule comprises a first nucleic acid encoding a pair of guide RNAs comprising a first and second guide RNA selected from any one of the following pairs of guide RNAs: SEQ ID NOs: 1020 and 23; 1023 and 23; 1023 and 1037; 1024 and 1055; 1025 and 23; 1025 and 1055; 1026 and 23; 1028 and 1055; 1029 and 1055; 1029 and 1037; 1031 and 1037; 1032 and 1037; 101029 and 1027; 1037 and 1048; 1037 and 1051; 1037 and 1053; 20 and 23; 1038 and 23; 21 and 23; 1040 and 23; 1042 and 1037; 1043 and 1037; 1044 and 1037; 1045 and 1037; 1046 and 1037; 24 and 1037; 1047 and 1055; or 1055 and 1022; and a second nucleic acid encoding a Staphylococcus aureus Cas9 (SaCas9).

In some embodiments, the payload comprises a nucleic acid molecule encoding one or more guide RNAs and a Cas9, wherein the nucleic acid molecule comprises a first nucleic acid encoding a pair of guide RNAs comprising a first and second guide RNA selected from any one of the following pairs of guide RNAs: SEQ ID NOs: 170 and 179; 172 and 179; 179 and 183; 179 and 185; 179 and 187; 179 and 188; 179 and 189; 179 and 193; 179 and 195; 179 and 196; 179 and 197; 200 and 174; or 200 and 176; and a second nucleic acid encoding a Staphylococcus lugdunensis (SluCas9).

In some embodiments, the payload comprises a nucleic acid molecule encoding one or more guide RNAs and a Cas9, wherein the nucleic acid molecule comprises a first nucleic acid encoding a pair of guide RNAs comprising a first and second guide RNA selected from any one of the following pairs of guide RNAs: SEQ ID NOs: 117 and 121; 117 and 122; 120 and 121; 120 and 123; 120 and 124; 120 and 125; 122 and 126; 122 and 123; 122 and 124; 122 and 125; or 122 and 126; and a second nucleic acid encoding a Staphylococcus lugdunensis (SluCas9); for targeting exon 44.

In some embodiments, the payload comprises a nucleic acid molecule encoding one or more guide RNAs and a Cas9, wherein the nucleic acid molecule comprises a first nucleic acid encoding a pair of guide RNAs comprising a first and second guide RNA selected from any one of the following pairs of guide RNAs: 155 and 156; 155 and 158; 155 and 162; 155 and 163; 162 and 157; 162 and 159; 162 and 164; 162 and 166; or 162 and 167; and a second nucleic acid encoding a Staphylococcus lugdunensis (SluCas9); for targeting exon 50.

In some embodiments, the payload comprises a nucleic acid molecule encoding one or more guide RNAs and a Cas9, wherein the nucleic acid molecule comprises a first nucleic acid encoding a pair of guide RNAs comprising a first and second guide RNA selected from any one of the following pairs of guide RNAs: SEQ ID NOs: 211 and 223; 211 and 225; 214 and 224; 216 and 223; 216 and 225; 220 and 224; 204 and 223; 223 and 224; or 204 and 225; and a second nucleic acid encoding a Staphylococcus lugdunensis (SluCas9); for targeting exon 53.

In some embodiments, the payload comprises a nucleic acid molecule encoding one or more guide RNAs and a Cas9, wherein the nucleic acid molecule comprises a first nucleic acid encoding a pair of guide RNAs comprising a first and second guide RNA selected from any one of the following pairs of guide RNAs: SEQ ID NOs: 1068 and 32; 1069 and 32; 1070 and 1075; 1071 and 32; 29 and 1075; 1072 and 27; 1072 and 28; 1072 and 32; 1072 and 33; 1073 and 1076; 1073 and 35; 221 and 1077; 1074 and 27; 1074 and 28; 1074 and 33; 32 and 1077; 1075 and 1076; 1075 and 35; 1076 and 26; or 35 and 26; and a second nucleic acid encoding a Staphylococcus aureus Cas9 (SaCas9); for targeting exon 53.

In some embodiments, the payload comprises a nucleic acid molecule encoding one or more guide RNAs and a Cas9, wherein the nucleic acid molecule comprises a first nucleic acid encoding a pair of guide RNAs comprising a first and second guide RNA selected from any one of the following pairs of guide RNAs: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9).

In some embodiments, the payload comprises a nucleic acid molecule encoding one or more guide RNAs and a Cas9, wherein the nucleic acid molecule comprises a first nucleic acid encoding a pair of guide RNAs comprising a first and second guide RNA selected from any one of the following pairs of guide RNAs: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9).

In general, in the case of a DNA nucleic acid construct encoding a guide RNA, the U residues in any of the RNA sequences described herein may be replaced with T residues, and in the case of a guide RNA construct encoded by a DNA, the T residues may be replaced with U residues.

TABLE 1A
Exemplary DMD guide sequences (human-hg38.p12)

| Sequence | | | | | |
|---|---|---|---|---|---|
| ID No. | | | | | |
| of Guide | | | | | |
| Sequence | EXON | CAS9 | strand | Guide sequence | pam |
| 1 | EXON43 | SACAS9 | + | GCAATGCTGCTGTCTTCTTGCT | ATGAAT |
| 2 | EXON43 | SACAS9 | − | AACAAAATGTACAAGGACCGAC | AAGGGT |
| 3 | EXON43 | SACAS9 | − | TGCAAAGTGCAACGCCTGTGGA | AAGGGT |
| 4 | EXON43 | SACAS9 | − | ATAGTCTACAACAAAGCTCAGG | TCGGAT |
| 5 | EXON43 | SACAS9 | − | CTGTTTTAAAATTTTTATATTA | CAGAAT |
| 6 | EXON44 | SACAS9 | + | ATTTAGCATGTTCCCAATTCTC | AGGAAT |
| 7 | EXON44 | SACAS9 | + | AATCGCCTGCAGGTAAAAGCAT | ATGGAT |
| 8 | EXON44 | SACAS9 | − | TCTCAGAAAGACACAAATTCCT | GAGAAT |
| 9 | EXON45 | SACAS9 | + | TCAGGCTTCCCAATTTTTCCTG | TAGAAT |
| 10 | EXON45 | SACAS9 | + | TAGAATACTGGCATCTGTTTTT | GAGGAT |
| 11 | EXON45 | SACAS9 | + | TGGCATCTGTTTTTGAGGATTG | CTGAAT |
| 12 | EXON45 | SACAS9 | + | TTGCCGCTGCCCAATGCCATCC | TGGAGT |
| 13 | EXON45 | SACAS9 | − | GAGGTAGGGCGACAGATCTAAT | AGGAAT |
| 14 | EXON45 | SACAS9 | − | TCTACAGGAAAAATTGGGAAGC | CTGAAT |
| 15 | EXON45 | SACAS9 | − | GCGGCAAACTGTTGTCAGAACA | TTGAAT |
| 16 | EXON45 | SACAS9 | − | TTTTGGTATCTTACAGGAACTC | CAGGAT |
| 17 | EXON50 | SACAS9 | − | ACTATTGGAGCCTGTAAGTATA | CTGGAT |
| 18 | EXON50 | SACAS9 | − | AGGAAGTTAGAAGATCTGAGCT | CTGAGT |
| 19 | EXON51 | SACAS9 | + | TAGTAACCACAGGTTGTGTCAC | CAGAGT |
| 20 | EXON51 | SACAS9 | + | GTTGTGTCACCAGAGTAACAGT | CTGAGT |
| 21 | EXON51 | SACAS9 | + | TCTGAGTAGGAGCTAAAATATT | TTGGGT |
| 22 | EXON51 | SACAS9 | − | GAGGGTGATGGTGGGTGACCTT | GAGGAT |
| 23 | EXON51 | SACAS9 | − | TATAAAATCACAGAGGGTGATG | GTGGGT |
| 24 | EXON51 | SACAS9 | − | TTGATCAAGTTATAAAATCACA | GAGGGT |
| 25 | EXON53 | SACAS9 | + | CCTTGGTTTCTGTGATTTTCTT | TTGGAT |
| 26 | EXON53 | SACAS9 | + | TCCTTAGCTTCCAGCCATTGTG | TTGAAT |
| 27 | EXON53 | SACAS9 | + | CTTGTACTTCATCCCACTGATT | CTGAAT |
| 28 | EXON53 | SACAS9 | + | ACTGATTCTGAATTCTTTCAAC | TAGAAT |
| 29 | EXON53 | SACAS9 | − | AGCCAAGCTTGAGTCATGGAAG | GAGGGT |
| 30 | EXON53 | SACAS9 | − | TTAGGACAGGCCAGAGCCAAGC | TTGAGT |
| 31 | EXON53 | SACAS9 | − | GCAACAGTTGAATGAAATGTTA | AAGGAT |
| 32 | EXON53 | SACAS9 | − | CCTTCAGAACCGGAGGCAACAG | TTGAAT |
| 33 | EXON53 | SACAS9 | − | AGTTGAAAGAATTCAGAATCAG | TGGGAT |
| 34 | EXON53 | SACAS9 | − | TTTTATTCTAGTTGAAAGAATT | CAGAAT |
| 35 | EXON53 | SACAS9 | − | TTTTTCCTTTTATTCTAGTTGA | AAGAAT |
| 100 | EXON43 | SLUCAS9 | + | ATATATGTGTTACCTACCCTTG | TCGG |
| 101 | EXON43 | SLUCAS9 | + | ACATTTTGTTAACTTTTTCCCA | TTGG |
| 102 | EXON43 | SLUCAS9 | + | CTTTTTCCCATTGGAAATCAAG | CTGG |
| 103 | EXON43 | SLUCAS9 | + | TTTTTCCCATTGGAAATCAAGC | TGGG |
| 104 | EXON43 | SLUCAS9 | + | TCCTGTAGCTTCACCCTTTCCA | CAGG |
| 105 | EXON43 | SLUCAS9 | − | AATGTACAAGGACCGACAAGGG | TAGG |
| 106 | EXON43 | SLUCAS9 | − | ACAAAATGTACAAGGACCGACA | AGGG |
| 107 | EXON43 | SLUCAS9 | − | AACAAAATGTACAAGGACCGAC | AAGG |
| 108 | EXON43 | SLUCAS9 | − | GGAAAAAGTTAACAAAATGTAC | AAGG |
| 109 | EXON43 | SLUCAS9 | − | TCTCTCCCAGCTTGATTTCCAA | TGGG |
| 110 | EXON43 | SLUCAS9 | − | CTCTCTCCCAGCTTGATTTCCA | ATGG |
| 111 | EXON43 | SLUCAS9 | − | GCCTGTGGAAAGGGTGAAGCTA | CAGG |
| 112 | EXON43 | SLUCAS9 | − | GCAAAGTGCAACGCCTGTGGAA | AGGG |
| 113 | EXON43 | SLUCAS9 | − | TGCAAAGTGCAACGCCTGTGGA | AAGG |
| 114 | EXON43 | SLUCAS9 | − | AGCATTGCAAAGTGCAACGCCT | GTGG |
| 115 | EXON43 | SLUCAS9 | − | ATAGTCTACAACAAAGCTCAGG | TCGG |
| 116 | EXON43 | SLUCAS9 | − | AAAGATAGTCTACAACAAAGCT | CAGG |
| 117 | EXON44 | SLUCAS9 | + | TATTTAGCATGTTCCCAATTCT | CAGG |
| 118 | EXON44 | SLUCAS9 | + | AACAGATCTGTCAAATCGCCTG | CAGG |
| 119 | EXON44 | SLUCAS9 | + | AATCGCCTGCAGGTAAAAGCAT | ATGG |
| 120 | EXON44 | SLUCAS9 | − | TGCTAAATACAAATGGTATCTT | AAGG |
| 121 | EXON44 | SLUCAS9 | − | ATTGGGAACATGCTAAATACAA | ATGG |
| 122 | EXON44 | SLUCAS9 | − | AAAGACACAAATTCCTGAGAAT | TGGG |
| 123 | EXON44 | SLUCAS9 | − | GAAAGACACAAATTCCTGAGAA | TTGG |
| 124 | EXON44 | SLUCAS9 | − | ATGATATAAAGATATTTAATCA | GTGG |
| 125 | EXON44 | SLUCAS9 | − | TTGACAGATCTGTTGAGAAATG | GCGG |
| 126 | EXON44 | SLUCAS9 | − | GATTTGACAGATCTGTTGAGAA | ATGG |
| 127 | EXON44 | SLUCAS9 | − | TTGATCCATATGCTTTTACCTG | CAGG |
| 128 | EXON45 | SLUCAS9 | + | AGACCTCCTGCCACCGCAGATT | CAGG |
| 129 | EXON45 | SLUCAS9 | + | TCCCAATTTTTCCTGTAGAATA | CTGG |
| 130 | EXON45 | SLUCAS9 | + | TAGAATACTGGCATCTGTTTTT | GAGG |
| 131 | EXON45 | SLUCAS9 | + | TTTGCCGCTGCCCAATGCCATC | CTGG |
| 132 | EXON45 | SLUCAS9 | + | GGAGTTCCTGTAAGATACCAAA | AAGG |
| 133 | EXON45 | SLUCAS9 | − | AGAGGTAGGGCGACAGATCTAA | TAGG |
| 134 | EXON45 | SLUCAS9 | − | CTGTCAGACAGAAAAAAGAGGT | AGGG |
| 135 | EXON45 | SLUCAS9 | − | GCTGTCAGACAGAAAAAAGAGG | TAGG |
| 136 | EXON45 | SLUCAS9 | − | AACAGCTGTCAGACAGAAAAAA | GAGG |
| 137 | EXON45 | SLUCAS9 | − | AAGCCTGAATCTGCGGTGGCAG | GAGG |
| 138 | EXON45 | SLUCAS9 | − | GGGAAGCCTGAATCTGCGGTGG | CAGG |
| 139 | EXON45 | SLUCAS9 | − | AATTGGGAAGCCTGAATCTGCG | GTGG |
| 140 | EXON45 | SLUCAS9 | − | AAAAATTGGGAAGCCTGAATCT | GCGG |
| 141 | EXON45 | SLUCAS9 | − | GCCAGTATTCTACAGGAAAAAT | TGGG |
| 142 | EXON45 | SLUCAS9 | − | TGCCAGTATTCTACAGGAAAAA | TTGG |
| 143 | EXON45 | SLUCAS9 | − | AAAAACAGATGCCAGTATTCTA | CAGG |
| 144 | EXON45 | SLUCAS9 | − | TGTCAGAACATTGAATGCAACT | GGGG |
| 145 | EXON45 | SLUCAS9 | − | TTGTCAGAACATTGAATGCAAC | TGGG |
| 146 | EXON45 | SLUCAS9 | − | GTTGTCAGAACATTGAATGCAA | CTGG |
| 147 | EXON45 | SLUCAS9 | − | AACTCCAGGATGGCATTGGGCA | GCGG |
| 148 | EXON45 | SLUCAS9 | − | TACAGGAACTCCAGGATGGCAT | TGGG |
| 149 | EXON45 | SLUCAS9 | − | TTACAGGAACTCCAGGATGGCA | TTGG |
| 150 | EXON45 | SLUCAS9 | − | GGTATCTTACAGGAACTCCAGG | ATGG |
| 151 | EXON45 | SLUCAS9 | − | TTTTGGTATCTTACAGGAACTC | CAGG |
| 152 | EXON45 | SLUCAS9 | − | GTTTTGCCTTTTTGGTATCTTA | CAGG |
| 153 | EXON45 | SLUCAS9 | − | TCTTTTCTCAAATAAAAAGACA | TGGG |
| 154 | EXON50 | SLUCAS9 | + | AGAATGGGATCCAGTATACTTA | CAGG |
| 155 | EXON50 | SLUCAS9 | + | AGTATACTTACAGGCTCCAATA | GTGG |
| 156 | EXON50 | SLUCAS9 | + | CAGGCTCCAATAGTGGTCAGTC | CAGG |
| 157 | EXON50 | SLUCAS9 | + | CAATAGTGGTCAGTCCAGGAGC | TAGG |
| 158 | EXON50 | SLUCAS9 | + | GTGGTCAGTCCAGGAGCTAGGT | CAGG |
| 159 | EXON50 | SLUCAS9 | + | TTGCCCTCAGCTCTTGAAGTAA | ACGG |
| 160 | EXON50 | SLUCAS9 | − | AGTATACTGGATCCCATTCTCT | TTGG |
| 161 | EXON50 | SLUCAS9 | − | ACTATTGGAGCCTGTAAGTATA | CTGG |
| 162 | EXON50 | SLUCAS9 | − | CTAGCTCCTGGACTGACCACTA | TTGG |
| 163 | EXON50 | SLUCAS9 | − | GCAAAGCAGCCTGACCTAGCTC | CTGG |
| 164 | EXON50 | SLUCAS9 | − | AAACCGTTTACTTCAAGAGCTG | AGGG |
| 165 | EXON50 | SLUCAS9 | − | TAAACCGTTTACTTCAAGAGCT | GAGG |
| 166 | EXON50 | SLUCAS9 | − | AGATCTGAGCTCTGAGTGGAAG | GCGG |
| 167 | EXON50 | SLUCAS9 | − | AGAAGATCTGAGCTCTGAGTGG | AAGG |
| 168 | EXON50 | SLUCAS9 | − | AGTTAGAAGATCTGAGCTCTGA | GTGG |
| 169 | EXON50 | SLUCAS9 | − | ATGTGTATGCTTTTCTGTTAAA | GAGG |
| 170 | EXON51 | SLUCAS9 | + | TGATCATCTCGTTGATATCCTC | AAGG |
| 171 | EXON51 | SLUCAS9 | + | TTGATCAAGCAGAGAAAGCCAG | TCGG |
| 172 | EXON51 | SLUCAS9 | + | AGTCGGTAAGTTCTGTCCAAGC | CCGG |
| 173 | EXON51 | SLUCAS9 | + | GCCCGGTTGAAATCTGCCAGAG | CAGG |
| 174 | EXON51 | SLUCAS9 | + | CAGAGCAGGTACCTCCAACATC | AAGG |
| 175 | EXON51 | SLUCAS9 | + | GGTACCTCCAACATCAAGGAAG | ATGG |
| 176 | EXON51 | SLUCAS9 | + | CAAGGAAGATGGCATTTCTAGT | TTGG |
| 177 | EXON51 | SLUCAS9 | + | AGATGGCATTTCTAGTTTGGAG | ATGG |
| 178 | EXON51 | SLUCAS9 | + | ATGGCAGTTTCCTTAGTAACCA | CAGG |
| 179 | EXON51 | SLUCAS9 | + | GTCACCAGAGTAACAGTCTGAG | TAGG |
| 180 | EXON51 | SLUCAS9 | + | TCTGAGTAGGAGCTAAAATATT | TTGG |
| 181 | EXON51 | SLUCAS9 | + | CTGAGTAGGAGCTAAAATATTT | TGGG |
| 182 | EXON51 | SLUCAS9 | + | AAATATTTTGGGTTTTTGCAAA | AAGG |
| 183 | EXON51 | SLUCAS9 | − | GTATGAGAAAAAATGATAAAAG | TTGG |
| 184 | EXON51 | SLUCAS9 | − | CAACGAGATGATCATCAAGCAG | AAGG |
| 185 | EXON51 | SLUCAS9 | − | GAGGGTGATGGTGGGTGACCTT | GAGG |
| 186 | EXON51 | SLUCAS9 | − | ATAAAATCACAGAGGGTGATGG | TGGG |
| 187 | EXON51 | SLUCAS9 | − | TATAAAATCACAGAGGGTGATG | GTGG |
| 188 | EXON51 | SLUCAS9 | − | AGTTATAAAATCACAGAGGGTG | ATGG |
| 189 | EXON51 | SLUCAS9 | − | TGATCAAGTTATAAAATCACAG | AGGG |
| 190 | EXON51 | SLUCAS9 | − | TTGATCAAGTTATAAAATCACA | GAGG |
| 191 | EXON51 | SLUCAS9 | − | GGGCTTGGACAGAACTTACCGA | CTGG |
| 192 | EXON51 | SLUCAS9 | − | CTCTGGCAGATTTCAACCGGGC | TTGG |
| 193 | EXON51 | SLUCAS9 | − | ACCTGCTCTGGCAGATTTCAAC | CGGG |
| 194 | EXON51 | SLUCAS9 | − | TACCTGCTCTGGCAGATTTCAA | CCGG |
| 195 | EXON51 | SLUCAS9 | − | CTTGATGTTGGAGGTACCTGCT | CTGG |
| 196 | EXON51 | SLUCAS9 | − | AATGCCATCTTCCTTGATGTTG | GAGG |
| 197 | EXON51 | SLUCAS9 | − | AGAAATGCCATCTTCCTTGATG | TTGG |
| 198 | EXON51 | SLUCAS9 | − | GGTGACACAACCTGTGGTTACT | AAGG |
| 199 | EXON51 | SLUCAS9 | − | TGTTACTCTGGTGACACAACCT | GTGG |
| 200 | EXON51 | SLUCAS9 | − | AGCTCCTACTCAGACTGTTACT | CTGG |
| 201 | EXON53 | SLUCAS9 | + | AAAGGTATCTTTGATACTAACC | TTGG |
| 202 | EXON53 | SLUCAS9 | + | CCTTGGTTTCTGTGATTTTCTT | TTGG |
| 203 | EXON53 | SLUCAS9 | + | CTTTTGGATTGCATCTACTGTA | TAGG |
| 204 | EXON53 | SLUCAS9 | + | TTTTGGATTGCATCTACTGTAT | AGGG |
| 205 | EXON53 | SLUCAS9 | + | ACCCTCCTTCCATGACTCAAGC | TTGG |
| 206 | EXON53 | SLUCAS9 | + | CTTCCATGACTCAAGCTTGGCT | CTGG |
| 207 | EXON53 | SLUCAS9 | + | ACATTTCATTCAACTGTTGCCT | CCGG |
| 208 | EXON53 | SLUCAS9 | + | TCAACTGTTGCCTCCGGTTCTG | AAGG |
| 209 | EXON53 | SLUCAS9 | + | TGAATTCTTTCAACTAGAATAA | AAGG |
| 210 | EXON53 | SLUCAS9 | − | CCAAAAGAAAATCACAGAAACC | AAGG |
| 211 | EXON53 | SLUCAS9 | − | GCCAAGCTTGAGTCATGGAAGG | AGGG |
| 212 | EXON53 | SLUCAS9 | − | AGCCAAGCTTGAGTCATGGAAG | GAGG |
| 213 | EXON53 | SLUCAS9 | − | CAGAGCCAAGCTTGAGTCATGG | AAGG |
| 214 | EXON53 | SLUCAS9 | − | AGGCCAGAGCCAAGCTTGAGTC | ATGG |
| 215 | EXON53 | SLUCAS9 | − | AGAAGCTGAGCAGGTCTTAGGA | CAGG |
| 216 | EXON53 | SLUCAS9 | − | AAGGAAGAAGCTGAGCAGGTCT | TAGG |
| 217 | EXON53 | SLUCAS9 | − | GGAAGCTAAGGAAGAAGCTGAG | CAGG |
| 218 | EXON53 | SLUCAS9 | − | TTCAACACAATGGCTGGAAGCT | AAGG |
| 219 | EXON53 | SLUCAS9 | − | GTTAAAGGATTCAACACAATGG | CTGG |
| 220 | EXON53 | SLUCAS9 | − | AAATGTTAAAGGATTCAACACA | ATGG |
| 221 | EXON53 | SLUCAS9 | − | GCAACAGTTGAATGAAATGTTA | AAGG |
| 222 | EXON53 | SLUCAS9 | − | TACAAGAACACCTTCAGAACCG | GAGG |
| 223 | EXON53 | SLUCAS9 | − | AAGTACAAGAACACCTTCAGAA | CCGG |
| 224 | EXON53 | SLUCAS9 | − | AGTTGAAAGAATTCAGAATCAG | TGGG |
| 225 | EXON53 | SLUCAS9 | − | TAGTTGAAAGAATTCAGAATCA | GTGG |

TABLE 1B
Exemplary DMD guide sequences (20-nucleotides and 21-nucleotides)

| Sequence | | | | |
|---|---|---|---|---|
| ID No. of | | | | |
| Guide | | | | |
| Sequence | EXON | CAS9 | Strand | Guide sequence |
| 3000 | EXON43 | SACAS9 | + | AATGCTGCTGTCTTCTTGCT |
| 3001 | EXON43 | SACAS9 | + | CAATGCTGCTGTCTTCTTGCT |
| 1 | EXON43 | SACAS9 | + | GCAATGCTGCTGTCTTCTTGCT |
| 3002 | EXON43 | SACAS9 | − | AACAAAATGTACAAGGACCG |
| 3003 | EXON43 | SACAS9 | − | AACAAAATGTACAAGGACCGA |
| 2 | EXON43 | SACAS9 | − | AACAAAATGTACAAGGACCGAC |
| 3004 | EXON43 | SACAS9 | − | TGCAAAGTGCAACGCCTGTG |
| 3005 | EXON43 | SACAS9 | − | TGCAAAGTGCAACGCCTGTGG |
| 3 | EXON43 | SACAS9 | − | TGCAAAGTGCAACGCCTGTGGA |
| 3006 | EXON43 | SACAS9 | − | ATAGTCTACAACAAAGCTCA |
| 3007 | EXON43 | SACAS9 | − | ATAGTCTACAACAAAGCTCAG |
| 4 | EXON43 | SACAS9 | − | ATAGTCTACAACAAAGCTCAGG |
| 3008 | EXON43 | SACAS9 | − | CTGTTTTAAAATTTTTATAT |
| 3009 | EXON43 | SACAS9 | − | CTGTTTTAAAATTTTTATATT |
| 5 | EXON43 | SACAS9 | − | CTGTTTTAAAATTTTTATATTA |
| 3010 | EXON44 | SACAS9 | + | TTAGCATGTTCCCAATTCTC |
| 3011 | EXON44 | SACAS9 | + | TTTAGCATGTTCCCAATTCTC |
| 6 | EXON44 | SACAS9 | + | ATTTAGCATGTTCCCAATTCTC |
| 3012 | EXON44 | SACAS9 | + | TCGCCTGCAGGTAAAAGCAT |
| 3013 | EXON44 | SACAS9 | + | ATCGCCTGCAGGTAAAAGCAT |
| 7 | EXON44 | SACAS9 | + | AATCGCCTGCAGGTAAAAGCAT |
| 3014 | EXON44 | SACAS9 | − | TCTCAGAAAGACACAAATTC |
| 3015 | EXON44 | SACAS9 | − | TCTCAGAAAGACACAAATTCC |
| 8 | EXON44 | SACAS9 | − | TCTCAGAAAGACACAAATTCCT |
| 3016 | EXON45 | SACAS9 | + | AGGCTTCCCAATTTTTCCTG |
| 3017 | EXON45 | SACAS9 | + | CAGGCTTCCCAATTTTTCCTG |
| 9 | EXON45 | SACAS9 | + | TCAGGCTTCCCAATTTTTCCTG |
| 3018 | EXON45 | SACAS9 | + | GAATACTGGCATCTGTTTTT |
| 3019 | EXON45 | SACAS9 | + | AGAATACTGGCATCTGTTTTT |
| 10 | EXON45 | SACAS9 | + | TAGAATACTGGCATCTGTTTTT |
| 3020 | EXON45 | SACAS9 | + | GCATCTGTTTTTGAGGATTG |
| 3021 | EXON45 | SACAS9 | + | GGCATCTGTTTTTGAGGATTG |
| 11 | EXON45 | SACAS9 | + | TGGCATCTGTTTTTGAGGATTG |
| 3022 | EXON45 | SACAS9 | + | GCCGCTGCCCAATGCCATCC |
| 3023 | EXON45 | SACAS9 | + | TGCCGCTGCCCAATGCCATCC |
| 12 | EXON45 | SACAS9 | + | TTGCCGCTGCCCAATGCCATCC |
| 3024 | EXON45 | SACAS9 | − | GAGGTAGGGCGACAGATCTA |
| 3025 | EXON45 | SACAS9 | − | GAGGTAGGGCGACAGATCTAA |
| 13 | EXON45 | SACAS9 | − | GAGGTAGGGCGACAGATCTAAT |
| 3026 | EXON45 | SACAS9 | − | TCTACAGGAAAAATTGGGAA |
| 3027 | EXON45 | SACAS9 | − | TCTACAGGAAAAATTGGGAAG |
| 14 | EXON45 | SACAS9 | − | TCTACAGGAAAAATTGGGAAGC |
| 3028 | EXON45 | SACAS9 | − | GCGGCAAACTGTTGTCAGAA |
| 3029 | EXON45 | SACAS9 | − | GCGGCAAACTGTTGTCAGAAC |
| 15 | EXON45 | SACAS9 | − | GCGGCAAACTGTTGTCAGAACA |
| 3030 | EXON45 | SACAS9 | − | TTTTGGTATCTTACAGGAAC |
| 3031 | EXON45 | SACAS9 | − | TTTTGGTATCTTACAGGAACT |
| 16 | EXON45 | SACAS9 | − | TTTTGGTATCTTACAGGAACTC |
| 3032 | EXON50 | SACAS9 | − | ACTATTGGAGCCTGTAAGTA |
| 3033 | EXON50 | SACAS9 | − | ACTATTGGAGCCTGTAAGTAT |
| 17 | EXON50 | SACAS9 | − | ACTATTGGAGCCTGTAAGTATA |
| 3034 | EXON50 | SACAS9 | − | AGGAAGTTAGAAGATCTGAG |
| 3035 | EXON50 | SACAS9 | − | AGGAAGTTAGAAGATCTGAGC |
| 18 | EXON50 | SACAS9 | − | AGGAAGTTAGAAGATCTGAGCT |
| 3036 | EXON51 | SACAS9 | + | GTAACCACAGGTTGTGTCAC |
| 3037 | EXON51 | SACAS9 | + | AGTAACCACAGGTTGTGTCAC |
| 19 | EXON51 | SACAS9 | + | TAGTAACCACAGGTTGTGTCAC |
| 3038 | EXON51 | SACAS9 | + | TGTGTCACCAGAGTAACAGT |
| 3039 | EXON51 | SACAS9 | + | TTGTGTCACCAGAGTAACAGT |
| 20 | EXON51 | SACAS9 | + | GTTGTGTCACCAGAGTAACAGT |
| 3040 | EXON51 | SACAS9 | + | TGAGTAGGAGCTAAAATATT |
| 3041 | EXON51 | SACAS9 | + | CTGAGTAGGAGCTAAAATATT |
| 21 | EXON51 | SACAS9 | + | TCTGAGTAGGAGCTAAAATATT |
| 3042 | EXON51 | SACAS9 | − | GAGGGTGATGGTGGGTGACC |
| 3043 | EXON51 | SACAS9 | − | GAGGGTGATGGTGGGTGACCT |
| 22 | EXON51 | SACAS9 | − | GAGGGTGATGGTGGGTGACCTT |
| 3044 | EXON51 | SACAS9 | − | TATAAAATCACAGAGGGTGA |
| 3045 | EXON51 | SACAS9 | − | TATAAAATCACAGAGGGTGAT |
| 23 | EXON51 | SACAS9 | − | TATAAAATCACAGAGGGTGATG |
| 3046 | EXON51 | SACAS9 | − | TTGATCAAGTTATAAAATCA |
| 3047 | EXON51 | SACAS9 | − | TTGATCAAGTTATAAAATCAC |
| 24 | EXON51 | SACAS9 | − | TTGATCAAGTTATAAAATCACA |
| 3048 | EXON53 | SACAS9 | + | TTGGTTTCTGTGATTTTCTT |
| 3049 | EXON53 | SACAS9 | + | CTTGGTTTCTGTGATTTTCTT |
| 25 | EXON53 | SACAS9 | + | CCTTGGTTTCTGTGATTTTCTT |
| 3050 | EXON53 | SACAS9 | + | CTTAGCTTCCAGCCATTGTG |
| 3051 | EXON53 | SACAS9 | + | CCTTAGCTTCCAGCCATTGTG |
| 26 | EXON53 | SACAS9 | + | TCCTTAGCTTCCAGCCATTGTG |
| 3052 | EXON53 | SACAS9 | + | TGTACTTCATCCCACTGATT |
| 3053 | EXON53 | SACAS9 | + | TTGTACTTCATCCCACTGATT |
| 27 | EXON53 | SACAS9 | + | CTTGTACTTCATCCCACTGATT |
| 3054 | EXON53 | SACAS9 | + | TGATTCTGAATTCTTTCAAC |
| 3055 | EXON53 | SACAS9 | + | CTGATTCTGAATTCTTTCAAC |
| 28 | EXON53 | SACAS9 | + | ACTGATTCTGAATTCTTTCAAC |
| 3056 | EXON53 | SACAS9 | − | AGCCAAGCTTGAGTCATGGA |
| 3057 | EXON53 | SACAS9 | − | AGCCAAGCTTGAGTCATGGAA |
| 29 | EXON53 | SACAS9 | − | AGCCAAGCTTGAGTCATGGAAG |
| 3058 | EXON53 | SACAS9 | − | TTAGGACAGGCCAGAGCCAA |
| 3059 | EXON53 | SACAS9 | − | TTAGGACAGGCCAGAGCCAAG |
| 30 | EXON53 | SACAS9 | − | TTAGGACAGGCCAGAGCCAAGC |
| 3060 | EXON53 | SACAS9 | − | GCAACAGTTGAATGAAATGT |
| 3061 | EXON53 | SACAS9 | − | GCAACAGTTGAATGAAATGTT |
| 31 | EXON53 | SACAS9 | − | GCAACAGTTGAATGAAATGTTA |
| 3062 | EXON53 | SACAS9 | − | CCTTCAGAACCGGAGGCAAC |
| 3063 | EXON53 | SACAS9 | − | CCTTCAGAACCGGAGGCAACA |
| 32 | EXON53 | SACAS9 | − | CCTTCAGAACCGGAGGCAACAG |
| 3064 | EXON53 | SACAS9 | − | AGTTGAAAGAATTCAGAATC |
| 3065 | EXON53 | SACAS9 | − | AGTTGAAAGAATTCAGAATCA |
| 33 | EXON53 | SACAS9 | − | AGTTGAAAGAATTCAGAATCAG |
| 3066 | EXON53 | SACAS9 | − | TTTTATTCTAGTTGAAAGAA |
| 3067 | EXON53 | SACAS9 | − | TTTTATTCTAGTTGAAAGAAT |
| 34 | EXON53 | SACAS9 | − | TTTTATTCTAGTTGAAAGAATT |
| 3068 | EXON53 | SACAS9 | − | TTTTTCCTTTTATTCTAGTT |
| 3069 | EXON53 | SACAS9 | − | TTTTTCCTTTTATTCTAGTTG |
| 35 | EXON53 | SACAS9 | − | TTTTTCCTTTTATTCTAGTTGA |
| 4000 | EXON43 | SLUCAS9 | + | ATATGTGTTACCTACCCTTG |
| 4001 | EXON43 | SLUCAS9 | + | TATATGTGTTACCTACCCTTG |
| 100 | EXON43 | SLUCAS9 | + | ATATATGTGTTACCTACCCTTG |
| 4002 | EXON43 | SLUCAS9 | + | ATTTTGTTAACTTTTTCCCA |
| 4003 | EXON43 | SLUCAS9 | + | CATTTTGTTAACTTTTTCCCA |
| 101 | EXON43 | SLUCAS9 | + | ACATTTTGTTAACTTTTTCCCA |
| 4004 | EXON43 | SLUCAS9 | + | TTTTCCCATTGGAAATCAAG |
| 4005 | EXON43 | SLUCAS9 | + | TTTTTCCCATTGGAAATCAAG |
| 102 | EXON43 | SLUCAS9 | + | CTTTTTCCCATTGGAAATCAAG |
| 4006 | EXON43 | SLUCAS9 | + | TTTCCCATTGGAAATCAAGC |
| 4007 | EXON43 | SLUCAS9 | + | TTTTCCCATTGGAAATCAAGC |
| 103 | EXON43 | SLUCAS9 | + | TTTTTCCCATTGGAAATCAAGC |
| 4008 | EXON43 | SLUCAS9 | + | CTGTAGCTTCACCCTTTCCA |
| 4009 | EXON43 | SLUCAS9 | + | CCTGTAGCTTCACCCTTTCCA |
| 104 | EXON43 | SLUCAS9 | + | TCCTGTAGCTTCACCCTTTCCA |
| 4010 | EXON43 | SLUCAS9 | − | AATGTACAAGGACCGACAAG |
| 4011 | EXON43 | SLUCAS9 | − | AATGTACAAGGACCGACAAGG |
| 105 | EXON43 | SLUCAS9 | − | AATGTACAAGGACCGACAAGGG |
| 4012 | EXON43 | SLUCAS9 | − | ACAAAATGTACAAGGACCGA |
| 4013 | EXON43 | SLUCAS9 | − | ACAAAATGTACAAGGACCGAC |
| 106 | EXON43 | SLUCAS9 | − | ACAAAATGTACAAGGACCGACA |
| 4014 | EXON43 | SLUCAS9 | − | AACAAAATGTACAAGGACCG |
| 4015 | EXON43 | SLUCAS9 | − | AACAAAATGTACAAGGACCGA |
| 107 | EXON43 | SLUCAS9 | − | AACAAAATGTACAAGGACCGAC |
| 4016 | EXON43 | SLUCAS9 | − | GGAAAAAGTTAACAAAATGT |
| 4017 | EXON43 | SLUCAS9 | − | GGAAAAAGTTAACAAAATGTA |
| 108 | EXON43 | SLUCAS9 | − | GGAAAAAGTTAACAAAATGTAC |
| 4018 | EXON43 | SLUCAS9 | − | TCTCTCCCAGCTTGATTTCC |
| 4019 | EXON43 | SLUCAS9 | − | TCTCTCCCAGCTTGATTTCCA |
| 109 | EXON43 | SLUCAS9 | − | TCTCTCCCAGCTTGATTTCCAA |
| 4020 | EXON43 | SLUCAS9 | − | CTCTCTCCCAGCTTGATTTC |
| 4021 | EXON43 | SLUCAS9 | − | CTCTCTCCCAGCTTGATTTCC |
| 110 | EXON43 | SLUCAS9 | − | CTCTCTCCCAGCTTGATTTCCA |
| 4022 | EXON43 | SLUCAS9 | − | GCCTGTGGAAAGGGTGAAGC |
| 4023 | EXON43 | SLUCAS9 | − | GCCTGTGGAAAGGGTGAAGCT |
| 111 | EXON43 | SLUCAS9 | − | GCCTGTGGAAAGGGTGAAGCTA |
| 4024 | EXON43 | SLUCAS9 | − | GCAAAGTGCAACGCCTGTGG |
| 4025 | EXON43 | SLUCAS9 | − | GCAAAGTGCAACGCCTGTGGA |
| 112 | EXON43 | SLUCAS9 | − | GCAAAGTGCAACGCCTGTGGAA |
| 4026 | EXON43 | SLUCAS9 | − | TGCAAAGTGCAACGCCTGTG |
| 4027 | EXON43 | SLUCAS9 | − | TGCAAAGTGCAACGCCTGTGG |
| 113 | EXON43 | SLUCAS9 | − | TGCAAAGTGCAACGCCTGTGGA |
| 4028 | EXON43 | SLUCAS9 | − | AGCATTGCAAAGTGCAACGC |
| 4029 | EXON43 | SLUCAS9 | − | AGCATTGCAAAGTGCAACGCC |
| 114 | EXON43 | SLUCAS9 | − | AGCATTGCAAAGTGCAACGCCT |
| 4030 | EXON43 | SLUCAS9 | − | ATAGTCTACAACAAAGCTCA |
| 4031 | EXON43 | SLUCAS9 | − | ATAGTCTACAACAAAGCTCAG |
| 115 | EXON43 | SLUCAS9 | − | ATAGTCTACAACAAAGCTCAGG |
| 4032 | EXON43 | SLUCAS9 | − | AAAGATAGTCTACAACAAAG |
| 4033 | EXON43 | SLUCAS9 | − | AAAGATAGTCTACAACAAAGC |
| 116 | EXON43 | SLUCAS9 | − | AAAGATAGTCTACAACAAAGCT |
| 4034 | EXON44 | SLUCAS9 | + | TTTAGCATGTTCCCAATTCT |
| 4035 | EXON44 | SLUCAS9 | + | ATTTAGCATGTTCCCAATTCT |
| 117 | EXON44 | SLUCAS9 | + | TATTTAGCATGTTCCCAATTCT |
| 4036 | EXON44 | SLUCAS9 | + | CAGATCTGTCAAATCGCCTG |
| 4037 | EXON44 | SLUCAS9 | + | ACAGATCTGTCAAATCGCCTG |
| 118 | EXON44 | SLUCAS9 | + | AACAGATCTGTCAAATCGCCTG |
| 4038 | EXON44 | SLUCAS9 | + | TCGCCTGCAGGTAAAAGCAT |
| 4039 | EXON44 | SLUCAS9 | + | ATCGCCTGCAGGTAAAAGCAT |
| 119 | EXON44 | SLUCAS9 | + | AATCGCCTGCAGGTAAAAGCAT |
| 4040 | EXON44 | SLUCAS9 | − | TGCTAAATACAAATGGTATC |
| 4041 | EXON44 | SLUCAS9 | − | TGCTAAATACAAATGGTATCT |
| 120 | EXON44 | SLUCAS9 | − | TGCTAAATACAAATGGTATCTT |
| 4042 | EXON44 | SLUCAS9 | − | ATTGGGAACATGCTAAATAC |
| 4043 | EXON44 | SLUCAS9 | − | ATTGGGAACATGCTAAATACA |
| 121 | EXON44 | SLUCAS9 | − | ATTGGGAACATGCTAAATACAA |
| 4044 | EXON44 | SLUCAS9 | − | AAAGACACAAATTCCTGAGA |
| 4045 | EXON44 | SLUCAS9 | − | AAAGACACAAATTCCTGAGAA |
| 122 | EXON44 | SLUCAS9 | − | AAAGACACAAATTCCTGAGAAT |
| 4046 | EXON44 | SLUCAS9 | − | GAAAGACACAAATTCCTGAG |
| 4047 | EXON44 | SLUCAS9 | − | GAAAGACACAAATTCCTGAGA |
| 123 | EXON44 | SLUCAS9 | − | GAAAGACACAAATTCCTGAGAA |
| 4048 | EXON44 | SLUCAS9 | − | ATGATATAAAGATATTTAAT |
| 4049 | EXON44 | SLUCAS9 | − | ATGATATAAAGATATTTAATC |
| 124 | EXON44 | SLUCAS9 | − | ATGATATAAAGATATTTAATCA |
| 4050 | EXON44 | SLUCAS9 | − | TTGACAGATCTGTTGAGAAA |
| 4051 | EXON44 | SLUCAS9 | − | TTGACAGATCTGTTGAGAAAT |
| 125 | EXON44 | SLUCAS9 | − | TTGACAGATCTGTTGAGAAATG |
| 4052 | EXON44 | SLUCAS9 | − | GATTTGACAGATCTGTTGAG |
| 4053 | EXON44 | SLUCAS9 | − | GATTTGACAGATCTGTTGAGA |
| 126 | EXON44 | SLUCAS9 | − | GATTTGACAGATCTGTTGAGAA |
| 4054 | EXON44 | SLUCAS9 | − | TTGATCCATATGCTTTTACC |
| 4055 | EXON44 | SLUCAS9 | − | TTGATCCATATGCTTTTACCT |
| 127 | EXON44 | SLUCAS9 | − | TTGATCCATATGCTTTTACCTG |
| 4056 | EXON45 | SLUCAS9 | + | ACCTCCTGCCACCGCAGATT |
| 4057 | EXON45 | SLUCAS9 | + | GACCTCCTGCCACCGCAGATT |
| 128 | EXON45 | SLUCAS9 | + | AGACCTCCTGCCACCGCAGATT |
| 4058 | EXON45 | SLUCAS9 | + | CCAATTTTTCCTGTAGAATA |
| 4059 | EXON45 | SLUCAS9 | + | CCCAATTTTTCCTGTAGAATA |
| 129 | EXON45 | SLUCAS9 | + | TCCCAATTTTTCCTGTAGAATA |
| 4060 | EXON45 | SLUCAS9 | + | GAATACTGGCATCTGTTTTT |
| 4061 | EXON45 | SLUCAS9 | + | AGAATACTGGCATCTGTTTTT |
| 130 | EXON45 | SLUCAS9 | + | TAGAATACTGGCATCTGTTTTT |
| 4062 | EXON45 | SLUCAS9 | + | TGCCGCTGCCCAATGCCATC |
| 4063 | EXON45 | SLUCAS9 | + | TTGCCGCTGCCCAATGCCATC |
| 131 | EXON45 | SLUCAS9 | + | TTTGCCGCTGCCCAATGCCATC |
| 4064 | EXON45 | SLUCAS9 | + | AGTTCCTGTAAGATACCAAA |
| 4065 | EXON45 | SLUCAS9 | + | GAGTTCCTGTAAGATACCAAA |
| 132 | EXON45 | SLUCAS9 | + | GGAGTTCCTGTAAGATACCAAA |
| 4066 | EXON45 | SLUCAS9 | − | AGAGGTAGGGCGACAGATCT |
| 4067 | EXON45 | SLUCAS9 | − | AGAGGTAGGGCGACAGATCTA |
| 133 | EXON45 | SLUCAS9 | − | AGAGGTAGGGCGACAGATCTAA |
| 4068 | EXON45 | SLUCAS9 | − | CTGTCAGACAGAAAAAAGAG |
| 4069 | EXON45 | SLUCAS9 | − | CTGTCAGACAGAAAAAAGAGG |
| 134 | EXON45 | SLUCAS9 | − | CTGTCAGACAGAAAAAAGAGGT |
| 4070 | EXON45 | SLUCAS9 | − | GCTGTCAGACAGAAAAAAGA |
| 4071 | EXON45 | SLUCAS9 | − | GCTGTCAGACAGAAAAAAGAG |
| 135 | EXON45 | SLUCAS9 | − | GCTGTCAGACAGAAAAAAGAGG |
| 4072 | EXON45 | SLUCAS9 | − | AACAGCTGTCAGACAGAAAA |
| 4073 | EXON45 | SLUCAS9 | − | AACAGCTGTCAGACAGAAAAA |
| 136 | EXON45 | SLUCAS9 | − | AACAGCTGTCAGACAGAAAAAA |
| 4074 | EXON45 | SLUCAS9 | − | AAGCCTGAATCTGCGGTGGC |
| 4075 | EXON45 | SLUCAS9 | − | AAGCCTGAATCTGCGGTGGCA |
| 137 | EXON45 | SLUCAS9 | − | AAGCCTGAATCTGCGGTGGCAG |
| 4076 | EXON45 | SLUCAS9 | − | GGGAAGCCTGAATCTGCGGT |
| 4077 | EXON45 | SLUCAS9 | − | GGGAAGCCTGAATCTGCGGTG |
| 138 | EXON45 | SLUCAS9 | − | GGGAAGCCTGAATCTGCGGTGG |
| 4078 | EXON45 | SLUCAS9 | − | AATTGGGAAGCCTGAATCTG |
| 4079 | EXON45 | SLUCAS9 | − | AATTGGGAAGCCTGAATCTGC |
| 139 | EXON45 | SLUCAS9 | − | AATTGGGAAGCCTGAATCTGCG |
| 4080 | EXON45 | SLUCAS9 | − | AAAAATTGGGAAGCCTGAAT |
| 4081 | EXON45 | SLUCAS9 | − | AAAAATTGGGAAGCCTGAATC |
| 140 | EXON45 | SLUCAS9 | − | AAAAATTGGGAAGCCTGAATCT |
| 4082 | EXON45 | SLUCAS9 | − | GCCAGTATTCTACAGGAAAA |
| 4083 | EXON45 | SLUCAS9 | − | GCCAGTATTCTACAGGAAAAA |
| 141 | EXON45 | SLUCAS9 | − | GCCAGTATTCTACAGGAAAAAT |
| 4084 | EXON45 | SLUCAS9 | − | TGCCAGTATTCTACAGGAAA |
| 4085 | EXON45 | SLUCAS9 | − | TGCCAGTATTCTACAGGAAAA |
| 142 | EXON45 | SLUCAS9 | − | TGCCAGTATTCTACAGGAAAAA |
| 4086 | EXON45 | SLUCAS9 | − | AAAAACAGATGCCAGTATTC |
| 4087 | EXON45 | SLUCAS9 | − | AAAAACAGATGCCAGTATTCT |
| 143 | EXON45 | SLUCAS9 | − | AAAAACAGATGCCAGTATTCTA |
| 4088 | EXON45 | SLUCAS9 | − | TGTCAGAACATTGAATGCAA |
| 4089 | EXON45 | SLUCAS9 | − | TGTCAGAACATTGAATGCAAC |
| 144 | EXON45 | SLUCAS9 | − | TGTCAGAACATTGAATGCAACT |
| 4090 | EXON45 | SLUCAS9 | − | TTGTCAGAACATTGAATGCA |
| 4091 | EXON45 | SLUCAS9 | − | TTGTCAGAACATTGAATGCAA |
| 145 | EXON45 | SLUCAS9 | − | TTGTCAGAACATTGAATGCAAC |
| 4092 | EXON45 | SLUCAS9 | − | GTTGTCAGAACATTGAATGC |
| 4093 | EXON45 | SLUCAS9 | − | GTTGTCAGAACATTGAATGCA |
| 146 | EXON45 | SLUCAS9 | − | GTTGTCAGAACATTGAATGCAA |
| 4094 | EXON45 | SLUCAS9 | − | AACTCCAGGATGGCATTGGG |
| 4095 | EXON45 | SLUCAS9 | − | AACTCCAGGATGGCATTGGGC |
| 147 | EXON45 | SLUCAS9 | − | AACTCCAGGATGGCATTGGGCA |
| 4096 | EXON45 | SLUCAS9 | − | TACAGGAACTCCAGGATGGC |
| 4097 | EXON45 | SLUCAS9 | − | TACAGGAACTCCAGGATGGCA |
| 148 | EXON45 | SLUCAS9 | − | TACAGGAACTCCAGGATGGCAT |
| 4098 | EXON45 | SLUCAS9 | − | TTACAGGAACTCCAGGATGG |
| 4099 | EXON45 | SLUCAS9 | − | TTACAGGAACTCCAGGATGGC |
| 149 | EXON45 | SLUCAS9 | − | TTACAGGAACTCCAGGATGGCA |
| 4100 | EXON45 | SLUCAS9 | − | GGTATCTTACAGGAACTCCA |
| 4101 | EXON45 | SLUCAS9 | − | GGTATCTTACAGGAACTCCAG |
| 150 | EXON45 | SLUCAS9 | − | GGTATCTTACAGGAACTCCAGG |
| 4102 | EXON45 | SLUCAS9 | − | TTTTGGTATCTTACAGGAAC |
| 4103 | EXON45 | SLUCAS9 | − | TTTTGGTATCTTACAGGAACT |
| 151 | EXON45 | SLUCAS9 | − | TTTTGGTATCTTACAGGAACTC |
| 4104 | EXON45 | SLUCAS9 | − | GTTTTGCCTTTTTGGTATCT |
| 4105 | EXON45 | SLUCAS9 | − | GTTTTGCCTTTTTGGTATCTT |
| 152 | EXON45 | SLUCAS9 | − | GTTTTGCCTTTTTGGTATCTTA |
| 4106 | EXON45 | SLUCAS9 | − | TCTTTTCTCAAATAAAAAGA |
| 4107 | EXON45 | SLUCAS9 | − | TCTTTTCTCAAATAAAAAGAC |
| 153 | EXON45 | SLUCAS9 | − | TCTTTTCTCAAATAAAAAGACA |
| 4108 | EXON50 | SLUCAS9 | + | AATGGGATCCAGTATACTTA |
| 4109 | EXON50 | SLUCAS9 | + | GAATGGGATCCAGTATACTTA |
| 154 | EXON50 | SLUCAS9 | + | AGAATGGGATCCAGTATACTTA |
| 4110 | EXON50 | SLUCAS9 | + | TATACTTACAGGCTCCAATA |
| 4111 | EXON50 | SLUCAS9 | + | GTATACTTACAGGCTCCAATA |
| 155 | EXON50 | SLUCAS9 | + | AGTATACTTACAGGCTCCAATA |
| 4112 | EXON50 | SLUCAS9 | + | GGCTCCAATAGTGGTCAGTC |
| 4113 | EXON50 | SLUCAS9 | + | AGGCTCCAATAGTGGTCAGTC |
| 156 | EXON50 | SLUCAS9 | + | CAGGCTCCAATAGTGGTCAGTC |
| 4114 | EXON50 | SLUCAS9 | + | ATAGTGGTCAGTCCAGGAGC |
| 4115 | EXON50 | SLUCAS9 | + | AATAGTGGTCAGTCCAGGAGC |
| 157 | EXON50 | SLUCAS9 | + | CAATAGTGGTCAGTCCAGGAGC |
| 4116 | EXON50 | SLUCAS9 | + | GGTCAGTCCAGGAGCTAGGT |
| 4117 | EXON50 | SLUCAS9 | + | TGGTCAGTCCAGGAGCTAGGT |
| 158 | EXON50 | SLUCAS9 | + | GTGGTCAGTCCAGGAGCTAGGT |
| 4118 | EXON50 | SLUCAS9 | + | GCCCTCAGCTCTTGAAGTAA |
| 4119 | EXON50 | SLUCAS9 | + | TGCCCTCAGCTCTTGAAGTAA |
| 159 | EXON50 | SLUCAS9 | + | TTGCCCTCAGCTCTTGAAGTAA |
| 4120 | EXON50 | SLUCAS9 | − | AGTATACTGGATCCCATTCT |
| 4121 | EXON50 | SLUCAS9 | − | AGTATACTGGATCCCATTCTC |
| 160 | EXON50 | SLUCAS9 | − | AGTATACTGGATCCCATTCTCT |
| 4122 | EXON50 | SLUCAS9 | − | ACTATTGGAGCCTGTAAGTA |
| 4123 | EXON50 | SLUCAS9 | − | ACTATTGGAGCCTGTAAGTAT |
| 161 | EXON50 | SLUCAS9 | − | ACTATTGGAGCCTGTAAGTATA |
| 4124 | EXON50 | SLUCAS9 | − | CTAGCTCCTGGACTGACCAC |
| 4125 | EXON50 | SLUCAS9 | − | CTAGCTCCTGGACTGACCACT |
| 162 | EXON50 | SLUCAS9 | − | CTAGCTCCTGGACTGACCACTA |
| 4126 | EXON50 | SLUCAS9 | − | GCAAAGCAGCCTGACCTAGC |
| 4127 | EXON50 | SLUCAS9 | − | GCAAAGCAGCCTGACCTAGCT |
| 163 | EXON50 | SLUCAS9 | − | GCAAAGCAGCCTGACCTAGCTC |
| 4128 | EXON50 | SLUCAS9 | − | AAACCGTTTACTTCAAGAGC |
| 4129 | EXON50 | SLUCAS9 | − | AAACCGTTTACTTCAAGAGCT |
| 164 | EXON50 | SLUCAS9 | − | AAACCGTTTACTTCAAGAGCTG |
| 4130 | EXON50 | SLUCAS9 | − | TAAACCGTTTACTTCAAGAG |
| 4131 | EXON50 | SLUCAS9 | − | TAAACCGTTTACTTCAAGAGC |
| 165 | EXON50 | SLUCAS9 | − | TAAACCGTTTACTTCAAGAGCT |
| 4132 | EXON50 | SLUCAS9 | − | AGATCTGAGCTCTGAGTGGA |
| 4133 | EXON50 | SLUCAS9 | − | AGATCTGAGCTCTGAGTGGAA |
| 166 | EXON50 | SLUCAS9 | − | AGATCTGAGCTCTGAGTGGAAG |
| 4134 | EXON50 | SLUCAS9 | − | AGAAGATCTGAGCTCTGAGT |
| 4135 | EXON50 | SLUCAS9 | − | AGAAGATCTGAGCTCTGAGTG |
| 167 | EXON50 | SLUCAS9 | − | AGAAGATCTGAGCTCTGAGTGG |
| 4136 | EXON50 | SLUCAS9 | − | AGTTAGAAGATCTGAGCTCT |
| 4137 | EXON50 | SLUCAS9 | − | AGTTAGAAGATCTGAGCTCTG |
| 168 | EXON50 | SLUCAS9 | − | AGTTAGAAGATCTGAGCTCTGA |
| 4138 | EXON50 | SLUCAS9 | − | ATGTGTATGCTTTTCTGTTA |
| 4139 | EXON50 | SLUCAS9 | − | ATGTGTATGCTTTTCTGTTAA |
| 169 | EXON50 | SLUCAS9 | − | ATGTGTATGCTTTTCTGTTAAA |
| 4140 | EXON51 | SLUCAS9 | + | ATCATCTCGTTGATATCCTC |
| 4141 | EXON51 | SLUCAS9 | + | GATCATCTCGTTGATATCCTC |
| 170 | EXON51 | SLUCAS9 | + | TGATCATCTCGTTGATATCCTC |
| 4142 | EXON51 | SLUCAS9 | + | GATCAAGCAGAGAAAGCCAG |
| 4143 | EXON51 | SLUCAS9 | + | TGATCAAGCAGAGAAAGCCAG |
| 171 | EXON51 | SLUCAS9 | + | TTGATCAAGCAGAGAAAGCCAG |
| 4144 | EXON51 | SLUCAS9 | + | TCGGTAAGTTCTGTCCAAGC |
| 4145 | EXON51 | SLUCAS9 | + | GTCGGTAAGTTCTGTCCAAGC |
| 172 | EXON51 | SLUCAS9 | + | AGTCGGTAAGTTCTGTCCAAGC |
| 4146 | EXON51 | SLUCAS9 | + | CCGGTTGAAATCTGCCAGAG |
| 4147 | EXON51 | SLUCAS9 | + | CCCGGTTGAAATCTGCCAGAG |
| 173 | EXON51 | SLUCAS9 | + | GCCCGGTTGAAATCTGCCAGAG |
| 4148 | EXON51 | SLUCAS9 | + | GAGCAGGTACCTCCAACATC |
| 4149 | EXON51 | SLUCAS9 | + | AGAGCAGGTACCTCCAACATC |
| 174 | EXON51 | SLUCAS9 | + | CAGAGCAGGTACCTCCAACATC |
| 4150 | EXON51 | SLUCAS9 | + | TACCTCCAACATCAAGGAAG |
| 4151 | EXON51 | SLUCAS9 | + | GTACCTCCAACATCAAGGAAG |
| 175 | EXON51 | SLUCAS9 | + | GGTACCTCCAACATCAAGGAAG |
| 4152 | EXON51 | SLUCAS9 | + | AGGAAGATGGCATTTCTAGT |
| 4153 | EXON51 | SLUCAS9 | + | AAGGAAGATGGCATTTCTAGT |
| 176 | EXON51 | SLUCAS9 | + | CAAGGAAGATGGCATTTCTAGT |
| 4154 | EXON51 | SLUCAS9 | + | ATGGCATTTCTAGTTTGGAG |
| 4155 | EXON51 | SLUCAS9 | + | GATGGCATTTCTAGTTTGGAG |
| 177 | EXON51 | SLUCAS9 | + | AGATGGCATTTCTAGTTTGGAG |
| 4156 | EXON51 | SLUCAS9 | + | GGCAGTTTCCTTAGTAACCA |
| 4157 | EXON51 | SLUCAS9 | + | TGGCAGTTTCCTTAGTAACCA |
| 178 | EXON51 | SLUCAS9 | + | ATGGCAGTTTCCTTAGTAACCA |
| 4158 | EXON51 | SLUCAS9 | + | CACCAGAGTAACAGTCTGAG |
| 4159 | EXON51 | SLUCAS9 | + | TCACCAGAGTAACAGTCTGAG |
| 179 | EXON51 | SLUCAS9 | + | GTCACCAGAGTAACAGTCTGAG |
| 4160 | EXON51 | SLUCAS9 | + | TGAGTAGGAGCTAAAATATT |
| 4161 | EXON51 | SLUCAS9 | + | CTGAGTAGGAGCTAAAATATT |
| 180 | EXON51 | SLUCAS9 | + | TCTGAGTAGGAGCTAAAATATT |
| 4162 | EXON51 | SLUCAS9 | + | GAGTAGGAGCTAAAATATTT |
| 4163 | EXON51 | SLUCAS9 | + | TGAGTAGGAGCTAAAATATTT |
| 181 | EXON51 | SLUCAS9 | + | CTGAGTAGGAGCTAAAATATTT |
| 4164 | EXON51 | SLUCAS9 | + | ATATTTTGGGTTTTTGCAAA |
| 4165 | EXON51 | SLUCAS9 | + | AATATTTTGGGTTTTTGCAAA |
| 182 | EXON51 | SLUCAS9 | + | AAATATTTTGGGTTTTTGCAAA |
| 4166 | EXON51 | SLUCAS9 | − | GTATGAGAAAAAATGATAAA |
| 4167 | EXON51 | SLUCAS9 | − | GTATGAGAAAAAATGATAAAA |
| 183 | EXON51 | SLUCAS9 | − | GTATGAGAAAAAATGATAAAAG |
| 4168 | EXON51 | SLUCAS9 | − | CAACGAGATGATCATCAAGC |
| 4169 | EXON51 | SLUCAS9 | − | CAACGAGATGATCATCAAGCA |
| 184 | EXON51 | SLUCAS9 | − | CAACGAGATGATCATCAAGCAG |
| 4170 | EXON51 | SLUCAS9 | − | GAGGGTGATGGTGGGTGACC |
| 4171 | EXON51 | SLUCAS9 | − | GAGGGTGATGGTGGGTGACCT |
| 185 | EXON51 | SLUCAS9 | − | GAGGGTGATGGTGGGTGACCTT |
| 4172 | EXON51 | SLUCAS9 | − | ATAAAATCACAGAGGGTGAT |
| 4173 | EXON51 | SLUCAS9 | − | ATAAAATCACAGAGGGTGATG |
| 186 | EXON51 | SLUCAS9 | − | ATAAAATCACAGAGGGTGATGG |
| 4174 | EXON51 | SLUCAS9 | − | TATAAAATCACAGAGGGTGA |
| 4175 | EXON51 | SLUCAS9 | − | TATAAAATCACAGAGGGTGAT |
| 187 | EXON51 | SLUCAS9 | − | TATAAAATCACAGAGGGTGATG |
| 4176 | EXON51 | SLUCAS9 | − | AGTTATAAAATCACAGAGGG |
| 4177 | EXON51 | SLUCAS9 | − | AGTTATAAAATCACAGAGGGT |
| 188 | EXON51 | SLUCAS9 | − | AGTTATAAAATCACAGAGGGTG |
| 4178 | EXON51 | SLUCAS9 | − | TGATCAAGTTATAAAATCAC |
| 4179 | EXON51 | SLUCAS9 | − | TGATCAAGTTATAAAATCACA |
| 189 | EXON51 | SLUCAS9 | − | TGATCAAGTTATAAAATCACAG |
| 4180 | EXON51 | SLUCAS9 | − | TTGATCAAGTTATAAAATCA |
| 4181 | EXON51 | SLUCAS9 | − | TTGATCAAGTTATAAAATCAC |
| 190 | EXON51 | SLUCAS9 | − | TTGATCAAGTTATAAAATCACA |
| 4182 | EXON51 | SLUCAS9 | − | GGGCTTGGACAGAACTTACC |
| 4183 | EXON51 | SLUCAS9 | − | GGGCTTGGACAGAACTTACCG |
| 191 | EXON51 | SLUCAS9 | − | GGGCTTGGACAGAACTTACCGA |
| 4184 | EXON51 | SLUCAS9 | − | CTCTGGCAGATTTCAACCGG |
| 4185 | EXON51 | SLUCAS9 | − | CTCTGGCAGATTTCAACCGGG |
| 192 | EXON51 | SLUCAS9 | − | CTCTGGCAGATTTCAACCGGGC |
| 4186 | EXON51 | SLUCAS9 | − | ACCTGCTCTGGCAGATTTCA |
| 4187 | EXON51 | SLUCAS9 | − | ACCTGCTCTGGCAGATTTCAA |
| 193 | EXON51 | SLUCAS9 | − | ACCTGCTCTGGCAGATTTCAAC |
| 4188 | EXON51 | SLUCAS9 | − | TACCTGCTCTGGCAGATTTC |
| 4189 | EXON51 | SLUCAS9 | − | TACCTGCTCTGGCAGATTTCA |
| 194 | EXON51 | SLUCAS9 | − | TACCTGCTCTGGCAGATTTCAA |
| 4190 | EXON51 | SLUCAS9 | − | CTTGATGTTGGAGGTACCTG |
| 4191 | EXON51 | SLUCAS9 | − | CTTGATGTTGGAGGTACCTGC |
| 195 | EXON51 | SLUCAS9 | − | CTTGATGTTGGAGGTACCTGCT |
| 4192 | EXON51 | SLUCAS9 | − | AATGCCATCTTCCTTGATGT |
| 4193 | EXON51 | SLUCAS9 | − | AATGCCATCTTCCTTGATGTT |
| 196 | EXON51 | SLUCAS9 | − | AATGCCATCTTCCTTGATGTTG |
| 4194 | EXON51 | SLUCAS9 | − | AGAAATGCCATCTTCCTTGA |
| 4195 | EXON51 | SLUCAS9 | − | AGAAATGCCATCTTCCTTGAT |
| 197 | EXON51 | SLUCAS9 | − | AGAAATGCCATCTTCCTTGATG |
| 4196 | EXON51 | SLUCAS9 | − | GGTGACACAACCTGTGGTTA |
| 4197 | EXON51 | SLUCAS9 | − | GGTGACACAACCTGTGGTTAC |
| 198 | EXON51 | SLUCAS9 | − | GGTGACACAACCTGTGGTTACT |
| 4198 | EXON51 | SLUCAS9 | − | TGTTACTCTGGTGACACAAC |
| 4199 | EXON51 | SLUCAS9 | − | TGTTACTCTGGTGACACAACC |
| 199 | EXON51 | SLUCAS9 | − | TGTTACTCTGGTGACACAACCT |
| 4200 | EXON51 | SLUCAS9 | − | AGCTCCTACTCAGACTGTTA |
| 4201 | EXON51 | SLUCAS9 | − | AGCTCCTACTCAGACTGTTAC |
| 200 | EXON51 | SLUCAS9 | − | AGCTCCTACTCAGACTGTTACT |
| 4202 | EXON53 | SLUCAS9 | + | AGGTATCTTTGATACTAACC |
| 4203 | EXON53 | SLUCAS9 | + | AAGGTATCTTTGATACTAACC |
| 201 | EXON53 | SLUCAS9 | + | AAAGGTATCTTTGATACTAACC |
| 4204 | EXON53 | SLUCAS9 | + | TTGGTTTCTGTGATTTTCTT |
| 4205 | EXON53 | SLUCAS9 | + | CTTGGTTTCTGTGATTTTCTT |
| 202 | EXON53 | SLUCAS9 | + | CCTTGGTTTCTGTGATTTTCTT |
| 4206 | EXON53 | SLUCAS9 | + | TTTGGATTGCATCTACTGTA |
| 4207 | EXON53 | SLUCAS9 | + | TTTTGGATTGCATCTACTGTA |
| 203 | EXON53 | SLUCAS9 | + | CTTTTGGATTGCATCTACTGTA |
| 4208 | EXON53 | SLUCAS9 | + | TTGGATTGCATCTACTGTAT |
| 4209 | EXON53 | SLUCAS9 | + | TTTGGATTGCATCTACTGTAT |
| 204 | EXON53 | SLUCAS9 | + | TTTTGGATTGCATCTACTGTAT |
| 4210 | EXON53 | SLUCAS9 | + | CCTCCTTCCATGACTCAAGC |
| 4211 | EXON53 | SLUCAS9 | + | CCCTCCTTCCATGACTCAAGC |
| 205 | EXON53 | SLUCAS9 | + | ACCCTCCTTCCATGACTCAAGC |
| 4212 | EXON53 | SLUCAS9 | + | TCCATGACTCAAGCTTGGCT |
| 4213 | EXON53 | SLUCAS9 | + | TTCCATGACTCAAGCTTGGCT |
| 206 | EXON53 | SLUCAS9 | + | CTTCCATGACTCAAGCTTGGCT |
| 4214 | EXON53 | SLUCAS9 | + | ATTTCATTCAACTGTTGCCT |
| 4215 | EXON53 | SLUCAS9 | + | CATTTCATTCAACTGTTGCCT |
| 207 | EXON53 | SLUCAS9 | + | ACATTTCATTCAACTGTTGCCT |
| 4216 | EXON53 | SLUCAS9 | + | AACTGTTGCCTCCGGTTCTG |
| 4217 | EXON53 | SLUCAS9 | + | CAACTGTTGCCTCCGGTTCTG |
| 208 | EXON53 | SLUCAS9 | + | TCAACTGTTGCCTCCGGTTCTG |
| 4218 | EXON53 | SLUCAS9 | + | AATTCTTTCAACTAGAATAA |
| 4219 | EXON53 | SLUCAS9 | + | GAATTCTTTCAACTAGAATAA |
| 209 | EXON53 | SLUCAS9 | + | TGAATTCTTTCAACTAGAATAA |
| 4220 | EXON53 | SLUCAS9 | − | CCAAAAGAAAATCACAGAAA |
| 4221 | EXON53 | SLUCAS9 | − | CCAAAAGAAAATCACAGAAAC |
| 210 | EXON53 | SLUCAS9 | − | CCAAAAGAAAATCACAGAAACC |
| 4222 | EXON53 | SLUCAS9 | − | GCCAAGCTTGAGTCATGGAA |
| 4223 | EXON53 | SLUCAS9 | − | GCCAAGCTTGAGTCATGGAAG |
| 211 | EXON53 | SLUCAS9 | − | GCCAAGCTTGAGTCATGGAAGG |
| 4224 | EXON53 | SLUCAS9 | − | AGCCAAGCTTGAGTCATGGA |
| 4225 | EXON53 | SLUCAS9 | − | AGCCAAGCTTGAGTCATGGAA |
| 212 | EXON53 | SLUCAS9 | − | AGCCAAGCTTGAGTCATGGAAG |
| 4226 | EXON53 | SLUCAS9 | − | CAGAGCCAAGCTTGAGTCAT |
| 4227 | EXON53 | SLUCAS9 | − | CAGAGCCAAGCTTGAGTCATG |
| 213 | EXON53 | SLUCAS9 | − | CAGAGCCAAGCTTGAGTCATGG |
| 4228 | EXON53 | SLUCAS9 | − | AGGCCAGAGCCAAGCTTGAG |
| 4229 | EXON53 | SLUCAS9 | − | AGGCCAGAGCCAAGCTTGAGT |
| 214 | EXON53 | SLUCAS9 | − | AGGCCAGAGCCAAGCTTGAGTC |
| 4230 | EXON53 | SLUCAS9 | − | AGAAGCTGAGCAGGTCTTAG |
| 4231 | EXON53 | SLUCAS9 | − | AGAAGCTGAGCAGGTCTTAGG |
| 215 | EXON53 | SLUCAS9 | − | AGAAGCTGAGCAGGTCTTAGGA |
| 4232 | EXON53 | SLUCAS9 | − | AAGGAAGAAGCTGAGCAGGT |
| 4233 | EXON53 | SLUCAS9 | − | AAGGAAGAAGCTGAGCAGGTC |
| 216 | EXON53 | SLUCAS9 | − | AAGGAAGAAGCTGAGCAGGTCT |
| 4234 | EXON53 | SLUCAS9 | − | GGAAGCTAAGGAAGAAGCTG |
| 4235 | EXON53 | SLUCAS9 | − | GGAAGCTAAGGAAGAAGCTGA |
| 217 | EXON53 | SLUCAS9 | − | GGAAGCTAAGGAAGAAGCTGAG |
| 4236 | EXON53 | SLUCAS9 | − | TTCAACACAATGGCTGGAAG |
| 4237 | EXON53 | SLUCAS9 | − | TTCAACACAATGGCTGGAAGC |
| 218 | EXON53 | SLUCAS9 | − | TTCAACACAATGGCTGGAAGCT |
| 4238 | EXON53 | SLUCAS9 | − | GTTAAAGGATTCAACACAAT |
| 4239 | EXON53 | SLUCAS9 | − | GTTAAAGGATTCAACACAATG |
| 219 | EXON53 | SLUCAS9 | − | GTTAAAGGATTCAACACAATGG |
| 4240 | EXON53 | SLUCAS9 | − | AAATGTTAAAGGATTCAACA |
| 4241 | EXON53 | SLUCAS9 | − | AAATGTTAAAGGATTCAACAC |
| 220 | EXON53 | SLUCAS9 | − | AAATGTTAAAGGATTCAACACA |
| 4242 | EXON53 | SLUCAS9 | − | GCAACAGTTGAATGAAATGT |
| 4243 | EXON53 | SLUCAS9 | − | GCAACAGTTGAATGAAATGTT |
| 221 | EXON53 | SLUCAS9 | − | GCAACAGTTGAATGAAATGTTA |
| 4244 | EXON53 | SLUCAS9 | − | TACAAGAACACCTTCAGAAC |
| 4245 | EXON53 | SLUCAS9 | − | TACAAGAACACCTTCAGAACC |
| 222 | EXON53 | SLUCAS9 | − | TACAAGAACACCTTCAGAACCG |
| 4246 | EXON53 | SLUCAS9 | − | AAGTACAAGAACACCTTCAG |
| 4247 | EXON53 | SLUCAS9 | − | AAGTACAAGAACACCTTCAGA |
| 223 | EXON53 | SLUCAS9 | − | AAGTACAAGAACACCTTCAGAA |
| 4248 | EXON53 | SLUCAS9 | − | AGTTGAAAGAATTCAGAATC |
| 4249 | EXON53 | SLUCAS9 | − | AGTTGAAAGAATTCAGAATCA |
| 224 | EXON53 | SLUCAS9 | − | AGTTGAAAGAATTCAGAATCAG |
| 4250 | EXON53 | SLUCAS9 | − | TAGTTGAAAGAATTCAGAAT |
| 4251 | EXON53 | SLUCAS9 | − | TAGTTGAAAGAATTCAGAATC |
| 225 | EXON53 | SLUCAS9 | − | TAGTTGAAAGAATTCAGAATCA |

TABLE 2
Additional DMD Guide Sequences (human-hg38.p12)

| | | | | Genomic | | | | | |
|---|---|---|---|---|---|---|---|---|---|
| | | | | coordinate | | SEQ | | | |
| | | | Genomic | chrX stop | | ID | | | |
| | | | coor- | (includes | | NO | | | |
| | | | dinate | PAM | | Guide | | | Off |
| | | | chrX_ | coor- | | se- | | | targets_ |
| EXON | CAS9 | ID | start | dinates) | Strand | quence | Guide sequence | PAM | grouped |
| 45 | SACAS9 | E45SaCas | 31968359 | 31968387 | + | 1000 | TGTTTGCAGACCTCCTGCCACC | GCAGAT | 20 |
| | KKH | 9KKH1 | | | | | | | |
| 45 | SACAS9 | E45SaCas | 31968373 | 31968401 | + | 1001 | CTGCCACCGCAGATTCAGGCTT | CCCAAT | 9 |
| | KKH | 9KKH2 | | | | | | | |
| 45 | SACAS9 | E45SaCas | 31968387 | 31968415 | + | 9 | TCAGGCTTCCCAATTTTTCCTG | TAGAAT | 37 |
| | KKH | 9KKH3 | | | | | | | |
| 45 | SACAS9 | E45SaCas | 31968409 | 31968437 | + | 10 | TAGAATACTGGCATCTGTTTTT | GAGGAT | 63 |
| | KKH | 9KKH4 | | | | | | | |
| 45 | SACAS9 | E45SaCas | 31968417 | 31968445 | + | 11 | TGGCATCTGTTTTTGAGGATTG | CTGAAT | 35 |
| | KKH | 9KKH5 | | | | | | | |
| 45 | SACAS9 | E45SaCas | 31968432 | 31968460 | + | 1002 | AGGATTGCTGAATTATTTCTTC | CCCAGT | 42 |
| | KKH | 9KKH6 | | | | | | | |
| 45 | SACAS9 | E45SaCas | 31968442 | 31968470 | + | 1003 | AATTATTTCTTCCCCAGTTGCA | TTCAAT | 68 |
| | KKH | 9KKH7 | | | | | | | |
| 45 | SACAS9 | E45SaCas | 31968456 | 31968484 | + | 1004 | CAGTTGCATTCAATGTTCTGAC | AACAGT | 17 |
| | KKH | 9KKH8 | | | | | | | |
| 45 | SACAS9 | E45SaCas | 31968471 | 31968499 | + | 1005 | TTCTGACAACAGTTTGCCGCTG | CCCAAT | 6 |
| | KKH | 9KKH9 | | | | | | | |
| 45 | SACAS9 | E45SaCas | 31968484 | 31968512 | + | 12 | TTGCCGCTGCCCAATGCCATCC | TGGAGT | 5 |
| | KKH | 9KKH10 | | | | | | | |
| 45 | SACAS9 | E45SaCas | 31968495 | 31968523 | + | 1006 | CAATGCCATCCTGGAGTTCCTG | TAAGAT | 27 |
| | KKH | 9KKH11 | | | | | | | |
| 45 | SACAS9 | E45SaCas | 31968517 | 31968545 | + | 1007 | TAAGATACCAAAAAGGCAAAAC | AAAAAT | 96 |
| | KKH | 9KKH12 | | | | | | | |
| 45 | SACAS9 | E45SaCas | 31968313 | 31968341 | − | 13 | GAGGTAGGGCGACAGATCTAAT | AGGAAT | 10 |
| | KKH | 9KKH13 | | | | | | | |
| 45 | SACAS9 | E45SaCas | 31968319 | 31968347 | − | 1008 | AAAAAAGAGGTAGGGCGACAGA | TCTAAT | 75 |
| | KKH | 9KKH14 | | | | | | | |
| 45 | SACAS9 | E45SaCas | 31968324 | 31968352 | − | 1009 | GACAGAAAAAAGAGGTAGGGCG | ACAGAT | 51 |
| | KKH | 9KKH15 | | | | | | | |
| 45 | SACAS9 | E45SaCas | 31968336 | 31968364 | − | 1010 | AAACAGCTGTCAGACAGAAAAA | AGAGGT | 123 |
| | KKH | 9KKH16 | | | | | | | |
| 45 | SACAS9 | E45SaCas | 31968368 | 31968396 | − | 1011 | GAAGCCTGAATCTGCGGTGGCA | GGAGGT | 19 |
| | KKH | 9KKH17 | | | | | | | |
| 45 | SACAS9 | E45SaCas | 31968378 | 31968406 | − | 1012 | GAAAAATTGGGAAGCCTGAATC | TGCGGT | 29 |
| | KKH | 9KKH18 | | | | | | | |
| 45 | SACAS9 | E45SaCas | 31968385 | 31968413 | − | 14 | TCTACAGGAAAAATTGGGAAGC | CTGAAT | 64 |
| | KKH | 9KKH19 | | | | | | | |
| 45 | SACAS9 | E45SaCas | 31968399 | 31968427 | − | 1013 | ACAGATGCCAGTATTCTACAGG | AAAAAT | 22 |
| | KKH | 9KKH20 | | | | | | | |
| 45 | SACAS9 | E45SaCas | 31968415 | 31968443 | − | 1014 | TCAGCAATCCTCAAAAACAGAT | GCCAGT | 50 |
| | KKH | 9KKH21 | | | | | | | |
| 45 | SACAS9 | E45SaCas | 31968421 | 31968449 | − | 1015 | AATAATTCAGCAATCCTCAAAA | ACAGAT | 99 |
| | KKH | 9KKH22 | | | | | | | |
| 45 | SACAS9 | E45SaCas | 31968435 | 31968463 | − | 1016 | GCAACTGGGGAAGAAATAATTC | AGCAAT | 43 |
| | KKH | 9KKH23 | | | | | | | |
| 45 | SACAS9 | E45SaCas | 31968443 | 31968471 | − | 1017 | CATTGAATGCAACTGGGGAAGA | AATAAT | 54 |
| | KKH | 9KKH24 | | | | | | | |
| 45 | SACAS9 | E45SaCas | 31968446 | 31968474 | − | 1018 | GAACATTGAATGCAACTGGGGA | AGAAAT | 23 |
| | KKH | 9KKH25 | | | | | | | |
| 45 | SACAS9 | E45SaCas | 31968463 | 31968491 | − | 15 | GCGGCAAACTGTTGTCAGAACA | TTGAAT | 5 |
| | KKH | 9KKH26 | | | | | | | |
| 45 | SACAS9 | E45SaCas | 31968502 | 31968530 | − | 16 | TTTTGGTATCTTACAGGAACTC | CAGGAT | 52 |
| | KKH | 9KKH27 | | | | | | | |
| 45 | SLUCAS | E45SLCas | 31968332 | 31968358 | + | 2000 | CCCTACCTCTTTTTTCTGTCTG | ACAG | 23 |
| | 9KH | 9KH1 | | | | | | | |
| 45 | SLUCAS | E45SLCas | 31968342 | 31968368 | + | 2001 | TTTTTCTGTCTGACAGCTGTTT | GCAG | 22 |
| | 9KH | 9KH2 | | | | | | | |
| 45 | SLUCAS | E45SLCas | 31968359 | 31968385 | + | 2002 | TGTTTGCAGACCTCCTGCCACC | GCAG | 9 |
| | 9KH | 9KH3 | | | | | | | |
| 45 | SLUCAS | E45SLCas | 31968365 | 31968391 | + | 2003 | CAGACCTCCTGCCACCGCAGAT | TCAG | 9 |
| | 9KH | 9KH4 | | | | | | | |
| 45 | SLUCAS | E45SLCas | 31968366 | 31968392 | + | 128 | AGACCTCCTGCCACCGCAGATT | CAGG | 10 |
| | 9KH | 9KH5 | | | | | | | |
| 45 | SLUCAS | E45SLCas | 31968386 | 31968412 | + | 2004 | TTCAGGCTTCCCAATTTTTCCT | GTAG | 11 |
| | 9KH | 9KH6 | | | | | | | |
| 45 | SLUCAS | E45SLCas | 31968394 | 31968420 | + | 129 | TCCCAATTTTTCCTGTAGAATA | CTGG | 19 |
| | 9KH | 9KH7 | | | | | | | |
| 45 | SLUCAS | E45SLCas | 31968408 | 31968434 | + | 2005 | GTAGAATACTGGCATCTGTTTT | TGAG | 16 |
| | 9KH | 9KH8 | | | | | | | |
| 45 | SLUCAS | E45SLCas | 31968409 | 31968435 | + | 130 | TAGAATACTGGCATCTGTTTTT | GAGG | 19 |
| | 9KH | 9KH9 | | | | | | | |
| 45 | SLUCAS | E45SLCas | 31968433 | 31968459 | + | 2006 | GGATTGCTGAATTATTTCTTCC | CCAG | 18 |
| | 9KH | 9KH10 | | | | | | | |
| 45 | SLUCAS | E45SLCas | 31968457 | 31968483 | + | 2007 | AGTTGCATTCAATGTTCTGACA | ACAG | 10 |
| | 9KH | 9KH11 | | | | | | | |
| 45 | SLUCAS | E45SLCas | 31968483 | 31968509 | + | 131 | TTTGCCGCTGCCCAATGCCATC | CTGG | 2 |
| | 9KH | 9KH12 | | | | | | | |
| 45 | SLUCAS | E45SLCas | 31968485 | 31968511 | + | 2008 | TGCCGCTGCCCAATGCCATCCT | GGAG | 7 |
| | 9KH | 9KH13 | | | | | | | |
| 45 | SLUCAS | E45SLCas | 31968495 | 31968521 | + | 2009 | CAATGCCATCCTGGAGTTCCTG | TAAG | 14 |
| | 9KH | 9KH14 | | | | | | | |
| 45 | SLUCAS | E45SLCas | 31968506 | 31968532 | + | 2010 | TGGAGTTCCTGTAAGATACCAA | AAAG | 10 |
| | 9KH | 9KH15 | | | | | | | |
| 45 | SLUCAS | E45SLCas | 31968507 | 31968533 | + | 132 | GGAGTTCCTGTAAGATACCAAA | AAGG | 9 |
| | 9KH | 9KH16 | | | | | | | |
| 45 | SLUCAS | E45SLCas | 31968316 | 31968342 | − | 133 | AGAGGTAGGGCGACAGATCTAA | TAGG | 6 |
| | 9KH | 9KH17 | | | | | | | |
| 45 | SLUCAS | E45SLCas | 31968317 | 31968343 | − | 2011 | AAGAGGTAGGGCGACAGATCTA | ATAG | 5 |
| | 9KH | 9KH18 | | | | | | | |
| 45 | SLUCAS | E45SLCas | 31968326 | 31968352 | − | 2012 | GACAGAAAAAAGAGGTAGGGCG | ACAG | 31 |
| | 9KH | 9KH19 | | | | | | | |
| 45 | SLUCAS | E45SLCas | 31968332 | 31968358 | − | 134 | CTGTCAGACAGAAAAAAGAGGT | AGGG | 28 |
| | 9KH | 9KH20 | | | | | | | |
| 45 | SLUCAS | E45SLCas | 31968333 | 31968359 | − | 135 | GCTGTCAGACAGAAAAAAGAGG | TAGG | 43 |
| | 9KH | 9KH21 | | | | | | | |
| 45 | SLUCAS | E45SLCas | 31968334 | 31968360 | − | 2013 | AGCTGTCAGACAGAAAAAAGAG | GTAG | 67 |
| | 9KH | 9KH22 | | | | | | | |
| 45 | SLUCAS | E45SLCas | 31968337 | 31968363 | − | 136 | AACAGCTGTCAGACAGAAAAAA | GAGG | 55 |
| | 9KH | 9KH23 | | | | | | | |
| 45 | SLUCAS | E45SLCas | 31968338 | 31968364 | − | 2014 | AAACAGCTGTCAGACAGAAAAA | AGAG | 37 |
| | 9KH | 9KH24 | | | | | | | |
| 45 | SLUCAS | E45SLCas | 31968340 | 31968366 | − | 2015 | GCAAACAGCTGTCAGACAGAAA | AAAG | 27 |
| | 9KH | 9KH25 | | | | | | | |
| 45 | SLUCAS | E45SLCas | 31968347 | 31968373 | − | 2016 | GAGGTCTGCAAACAGCTGTCAG | ACAG | 23 |
| | 9KH | 9KH26 | | | | | | | |
| 45 | SLUCAS | E45SLCas | 31968351 | 31968377 | − | 2017 | GCAGGAGGTCTGCAAACAGCTG | TCAG | 16 |
| | 9KH | 9KH27 | | | | | | | |
| 45 | SLUCAS | E45SLCas | 31968358 | 31968384 | − | 2018 | TGCGGTGGCAGGAGGTCTGCAA | ACAG | 14 |
| | 9KH | 9KH28 | | | | | | | |
| 45 | SLUCAS | E45SLCas | 31968369 | 31968395 | − | 137 | AAGCCTGAATCTGCGGTGGCAG | GAGG | 11 |
| | 9KH | 9KH29 | | | | | | | |
| 45 | SLUCAS | E45SLCas | 31968370 | 31968396 | − | 2019 | GAAGCCTGAATCTGCGGTGGCA | GGAG | 15 |
| | 9KH | 9KH30 | | | | | | | |
| 45 | SLUCAS | E45SLCas | 31968372 | 31968398 | − | 138 | GGGAAGCCTGAATCTGCGGTGG | CAGG | 8 |
| | 9KH | 9KH31 | | | | | | | |
| 45 | SLUCAS | E45SLCas | 31968373 | 31968399 | − | 2020 | TGGGAAGCCTGAATCTGCGGTG | GCAG | 6 |
| | 9KH | 9KH32 | | | | | | | |
| 45 | SLUCAS | E45SLCas | 31968376 | 31968402 | − | 139 | AATTGGGAAGCCTGAATCTGCG | GTGG | 7 |
| | 9KH | 9KH33 | | | | | | | |
| 45 | SLUCAS | E45SLCas | 31968379 | 31968405 | − | 140 | AAAAATTGGGAAGCCTGAATCT | GCGG | 26 |
| | 9KH | 9KH34 | | | | | | | |
| 45 | SLUCAS | E45SLCas | 31968392 | 31968418 | − | 2021 | AGTATTCTACAGGAAAAATTGG | GAAG | 28 |
| | 9KH | 9KH35 | | | | | | | |
| 45 | SLUCAS | E45SLCas | 31968395 | 31968421 | − | 141 | GCCAGTATTCTACAGGAAAAAT | TGGG | 16 |
| | 9KH | 9KH36 | | | | | | | |
| 45 | SLUCAS | E45SLCas | 31968396 | 31968422 | − | 142 | TGCCAGTATTCTACAGGAAAAA | TTGG | 17 |
| | 9KH | 9KH37 | | | | | | | |
| 45 | SLUCAS | E45SLCas | 31968405 | 31968431 | − | 143 | AAAAACAGATGCCAGTATTCTA | CAGG | 21 |
| | 9KH | 9KH38 | | | | | | | |
| 45 | SLUCAS | E45SLCas | 31968406 | 31968432 | − | 2022 | CAAAAACAGATGCCAGTATTCT | ACAG | 28 |
| | 9KH | 9KH39 | | | | | | | |
| 45 | SLUCAS | E45SLCas | 31968416 | 31968442 | − | 2023 | CAGCAATCCTCAAAAACAGATG | CCAG | 23 |
| | 9KH | 9KH40 | | | | | | | |
| 45 | SLUCAS | E45SLCas | 31968423 | 31968449 | − | 2024 | AATAATTCAGCAATCCTCAAAA | ACAG | 31 |
| | 9KH | 9KH41 | | | | | | | |
| 45 | SLUCAS | E45SLCas | 31968439 | 31968465 | − | 2025 | ATGCAACTGGGGAAGAAATAAT | TCAG | 33 |
| | 9KH | 9KH42 | | | | | | | |
| 45 | SLUCAS | E45SLCas | 31968450 | 31968476 | − | 2026 | CAGAACATTGAATGCAACTGGG | GAAG | 11 |
| | 9KH | 9KH43 | | | | | | | |
| 45 | SLUCAS | E45SLCas | 31968453 | 31968479 | − | 144 | TGTCAGAACATTGAATGCAACT | GGGG | 12 |
| | 9KH | 9KH44 | | | | | | | |
| 45 | SLUCAS | E45SLCas | 31968454 | 31968480 | − | 145 | TTGTCAGAACATTGAATGCAAC | TGGG | 30 |
| | 9KH | 9KH45 | | | | | | | |
| 45 | SLUCAS | E45SLCas | 31968455 | 31968481 | − | 146 | GTTGTCAGAACATTGAATGCAA | CTGG | 12 |
| | 9KH | 9KH46 | | | | | | | |
| 45 | SLUCAS | E45SLCas | 31968473 | 31968499 | − | 2027 | ATTGGGCAGCGGCAAACTGTTG | TCAG | 3 |
| | 9KH | 9KH47 | | | | | | | |
| 45 | SLUCAS | E45SLCas | 31968487 | 31968513 | − | 147 | AACTCCAGGATGGCATTGGGCA | GCGG | 9 |
| | 9KH | 9KH48 | | | | | | | |
| 45 | SLUCAS | E45SLCas | 31968490 | 31968516 | − | 2028 | AGGAACTCCAGGATGGCATTGG | GCAG | 16 |
| | 9KH | 9KH49 | | | | | | | |
| 45 | SLUCAS | E45SLCas | 31968493 | 31968519 | − | 148 | TACAGGAACTCCAGGATGGCAT | TGGG | 10 |
| | 9KH | 9KH50 | | | | | | | |
| 45 | SLUCAS | E45SLCas | 31968494 | 31968520 | − | 149 | TTACAGGAACTCCAGGATGGCA | TTGG | 13 |
| | 9KH | 9KH51 | | | | | | | |
| 45 | SLUCAS | E45SLCas | 31968500 | 31968526 | − | 150 | GGTATCTTACAGGAACTCCAGG | ATGG | 7 |
| | 9KH | 9KH52 | | | | | | | |
| 45 | SLUCAS | E45SLCas | 31968504 | 31968530 | − | 151 | TTTTGGTATCTTACAGGAACTC | CAGG | 35 |
| | 9KH | 9KH53 | | | | | | | |
| 45 | SLUCAS | E45SLCas | 31968505 | 31968531 | − | 2029 | TTTTTGGTATCTTACAGGAACT | CCAG | 21 |
| | 9KH | 9KH54 | | | | | | | |
| 45 | SLUCAS | E45SLCas | 31968513 | 31968539 | − | 152 | GTTTTGCCTTTTTGGTATCTTA | CAGG | 32 |
| | 9KH | 9KH55 | | | | | | | |
| 45 | SLUCAS | E45SLCas | 31968514 | 31968540 | − | 2030 | TGTTTTGCCTTTTTGGTATCTT | ACAG | 58 |
| | 9KH | 9KH56 | | | | | | | |
| 51 | SACAS9 | E51SaCas | 31773943 | 31773971 | + | 1019 | TCATTTTTTCTCATACCTTCTG | CTTGAT | 91 |
| | KKH | 9KKH1 | | | | | | | |
| 51 | SACAS9 | E51SaCas | 31773946 | 31773974 | + | 1020 | TTTTTTCTCATACCTTCTGCTT | GATGAT | 132 |
| | KKH | 9KKH2 | | | | | | | |
| 51 | SACAS9 | E51SaCas | 31773958 | 31773986 | + | 1021 | CCTTCTGCTTGATGATCATCTC | GTTGAT | 23 |
| | KKH | 9KKH3 | | | | | | | |
| 51 | SACAS9 | E51SaCas | 31773969 | 31773997 | + | 1022 | ATGATCATCTCGTTGATATCCT | CAAGGT | 15 |
| | KKH | 9KKH4 | | | | | | | |
| 51 | SACAS9 | E51SaCas | 31773992 | 31774020 | + | 1023 | AAGGTCACCCACCATCACCCTC | TGTGAT | 33 |
| | KKH | 9KKH5 | | | | | | | |
| 51 | SACAS9 | E51SaCas | 31774005 | 31774033 | + | 1024 | ATCACCCTCTGTGATTTTATAA | CTTGAT | 45 |
| | KKH | 9KKH6 | | | | | | | |
| 51 | SACAS9 | E51SaCas | 31774023 | 31774051 | + | 1025 | ATAACTTGATCAAGCAGAGAAA | GCCAGT | 101 |
| | KKH | 9KKH7 | | | | | | | |
| 51 | SACAS9 | E51SaCas | 31774027 | 31774055 | + | 1026 | CTTGATCAAGCAGAGAAAGCCA | GTCGGT | 55 |
| | KKH | 9KKH8 | | | | | | | |
| 51 | SACAS9 | E51SaCas | 31774031 | 31774059 | + | 1027 | ATCAAGCAGAGAAAGCCAGTCG | GTAAGT | 16 |
| | KKH | 9KKH9 | | | | | | | |
| 51 | SACAS9 | E51SaCas | 31774047 | 31774075 | + | 1028 | CAGTCGGTAAGTTCTGTCCAAG | CCCGGT | 7 |
| | KKH | 9KKH10 | | | | | | | |
| 51 | SACAS9 | E51SaCas | 31774053 | 31774081 | 1 | 1029 | GTAAGTTCTGTCCAAGCCCGGT | TGAAAT | 4 |
| | KKH | 9KKH11 | | | | | | | |
| 51 | SACAS9 | E51SaCas | 31774067 | 31774095 | + | 1030 | AGCCCGGTTGAAATCTGCCAGA | GCAGGT | 7 |
| | KKH | 9KKH12 | | | | | | | |
| 51 | SACAS9 | E51SaCas | 31774088 | 31774116 | + | 1031 | AGCAGGTACCTCCAACATCAAG | GAAGAT | 23 |
| | KKH | 9KKH13 | | | | | | | |
| 51 | SACAS9 | E51SaCas | 31774100 | 31774128 | + | 1032 | CAACATCAAGGAAGATGGCATT | TCTAGT | 54 |
| | KKH | 9KKH14 | | | | | | | |
| 51 | SACAS9 | E51SaCas | 31774108 | 31774136 | + | 1033 | AGGAAGATGGCATTTCTAGTTT | GGAGAT | 62 |
| | KKH | 9KKH15 | | | | | | | |
| 51 | SACAS9 | E51SaCas | 31774114 | 31774142 | + | 1034 | ATGGCATTTCTAGTTTGGAGAT | GGCAGT | 56 |
| | KKH | 9KKH16 | | | | | | | |
| 51 | SACAS9 | E51SaCas | 31774123 | 31774151 | + | 1035 | CTAGTTTGGAGATGGCAGTTTC | CTTAGT | 32 |
| | KKH | 9KKH17 | | | | | | | |
| 51 | SACAS9 | E51SaCas | 31774133 | 31774161 | + | 1036 | GATGGCAGTTTCCTTAGTAACC | ACAGGT | 24 |
| | KKH | 9KKH18 | | | | | | | |
| 51 | SACAS9 | E51SaCas | 31774147 | 31774175 | + | 19 | TAGTAACCACAGGTTGTGTCAC | CAGAGT | 18 |
| | KKH | 9KKH19 | | | | | | | |
| 51 | SACAS9 | E51SaCas | 31774153 | 31774181 | + | 1037 | CCACAGGTTGTGTCACCAGAGT | AACAGT | 20 |
| | KKH | 9KKH20 | | | | | | | |
| 51 | SACAS9 | E51SaCas | 31774159 | 31774187 | + | 20 | GTTGTGTCACCAGAGTAACAGT | CTGAGT | 21 |
| | KKH | 9KKH21 | | | | | | | |
| 51 | SACAS9 | E51SaCas | 31774171 | 31774199 | + | 1038 | GAGTAACAGTCTGAGTAGGAGC | TAAAAT | 18 |
| | KKH | 9KKH22 | | | | | | | |
| 51 | SACAS9 | E51SaCas | 31774180 | 31774208 | + | 21 | TCTGAGTAGGAGCTAAAATATT | TTGGGT | 38 |
| | KKH | 9KKH23 | | | | | | | |
| 51 | SACAS9 | E51SaCas | 31773936 | 31773964 | − | 1039 | AGAAGGTATGAGAAAAAATGAT | AAAAGT | 236 |
| | KKH | 9KKH24 | | | | | | | |
| 51 | SACAS9 | E51SaCas | 31773942 | 31773970 | − | 1040 | TCAAGCAGAAGGTATGAGAAAA | AATGAT | 99 |
| | KKH | 9KKH25 | | | | | | | |
| 51 | SACAS9 | E51SaCas | 31773945 | 31773973 | − | 1041 | TCATCAAGCAGAAGGTATGAGA | AAAAAT | 43 |
| | KKH | 9KKH26 | | | | | | | |
| 51 | SACAS9 | E51SaCas | 31773957 | 31773985 | − | 1042 | TCAACGAGATGATCATCAAGCA | GAAGGT | 10 |
| | KKH | 9KKH27 | | | | | | | |
| 51 | SACAS9 | E51SaCas | 31773972 | 31774000 | − | 1043 | GTGACCTTGAGGATATCAACGA | GATGAT | 7 |
| | KKH | 9KKH28 | | | | | | | |
| 51 | SACAS9 | E51SaCas | 31773975 | 31774003 | − | 1044 | TGGGTGACCTTGAGGATATCAA | CGAGAT | 68 |
| | KKH | 9KKH29 | | | | | | | |
| 51 | SACAS9 | E51SaCas | 31773986 | 31774014 | − | 22 | GAGGGTGATGGTGGGTGACCTT | GAGGAT | 46 |
| | KKH | 9KKH30 | | | | | | | |
| 51 | SACAS9 | E51SaCas | 31773998 | 31774026 | − | 23 | TATAAAATCACAGAGGGTGATG | GTGGGT | 42 |
| | KKH | 9KKH31 | | | | | | | |
| 51 | SACAS9 | E51SaCas | 31774002 | 31774030 | − | 1045 | AAGTTATAAAATCACAGAGGGT | GATGGT | 77 |
| | KKH | 9KKH32 | | | | | | | |
| 51 | SACAS9 | E51SaCas | 31774005 | 31774033 | | 1046 | ATCAAGTTATAAAATCACAGAG | GGTGAT | 90 |
| | KKH | 9KKH33 | | | | | | | |
| 51 | SACAS9 | E51SaCas | 31774008 | 31774036 | − | 24 | TTGATCAAGTTATAAAATCACA | GAGGGT | 86 |
| | KKH | 9KKH34 | | | | | | | |
| 51 | SACAS9 | E51SaCas | 31774018 | 31774046 | − | 1047 | CTTTCTCTGCTTGATCAAGTTA | TAAAAT | 45 |
| | KKH | 9KKH35 | | | | | | | |
| 51 | SACAS9 | E51SaCas | 31774026 | 31774054 | − | 1048 | CCGACTGGCTTTCTCTGCTTGA | TCAAGT | 18 |
| | KKH | 9KKH36 | | | | | | | |
| 51 | SACAS9 | E51SaCas | 31774031 | 31774059 | − | 1049 | ACTTACCGACTGGCTTTCTCTG | CTTGAT | 11 |
| | KKH | 9KKH37 | | | | | | | |
| 51 | SACAS9 | E51SaCas | 31774079 | 31774107 | − | 1050 | GATGTTGGAGGTACCTGCTCTG | GCAGAT | 22 |
| | KKH | 9KKH38 | | | | | | | |
| 51 | SACAS9 | E51SaCas | 31774095 | 31774123 | − | 1051 | AAATGCCATCTTCCTTGATGTT | GGAGGT | 57 |
| | KKH | 9KKH39 | | | | | | | |
| 51 | SACAS9 | E51SaCas | 31774104 | 31774132 | − | 1052 | CCAAACTAGAAATGCCATCTTC | CTTGAT | 46 |
| | KKH | 9KKH40 | | | | | | | |
| 51 | SACAS9 | E51SaCas | 31774119 | 31774147 | − | 1053 | AGGAAACTGCCATCTCCAAACT | AGAAAT | 63 |
| | KKH | 9KKH41 | | | | | | | |
| 51 | SACAS9 | E51SaCas | 31774152 | 31774180 | − | 1054 | CTGTTACTCTGGTGACACAACC | TGTGGT | 27 |
| | KKH | 9KKH42 | | | | | | | |
| 51 | SACAS9 | E51SaCas | 31774167 | 31774195 | − | 1055 | TAGCTCCTACTCAGACTGTTAC | TCTGGT | 13 |
| | KKH | 9KKH43 | | | | | | | |
| 51 | SLUCAS | E51SLCas | 31773969 | 31773995 | + | 2031 | ATGATCATCTCGTTGATATCCT | CAAG | 6 |
| | 9KH | 9KH1 | | | | | | | |
| 51 | SLUCAS | E51SLCas | 31773970 | 31773996 | + | 170 | TGATCATCTCGTTGATATCCTC | AAGG | 6 |
| | 9KH | 9KH2 | | | | | | | |
| 51 | SLUCAS | E51SLCas | 31774011 | 31774037 | + | 2032 | CTCTGTGATTTTATAACTTGAT | CAAG | 18 |
| | 9KH | 9KH3 | | | | | | | |
| 51 | SLUCAS | E51SLCas | 31774014 | 31774040 | + | 2033 | TGTGATTTTATAACTTGATCAA | GCAG | 29 |
| | 9KH | 9KH4 | | | | | | | |
| 51 | SLUCAS | E51SLCas | 31774016 | 31774042 | + | 2034 | TGATTTTATAACTTGATCAAGC | AGAG | 15 |
| | 9KH | 9KH5 | | | | | | | |
| 51 | SLUCAS | E51SLCas | 31774020 | 31774046 | + | 2035 | TTTATAACTTGATCAAGCAGAG | AAAG | 7 |
| | 9KH | 9KH6 | | | | | | | |
| 51 | SLUCAS | E51SLCas | 31774024 | 31774050 | + | 2036 | TAACTTGATCAAGCAGAGAAAG | CCAG | 24 |
| | 9KH | 9KH7 | | | | | | | |
| 51 | SLUCAS | E51SLCas | 31774028 | 31774054 | + | 171 | TTGATCAAGCAGAGAAAGCCAG | TCGG | 23 |
| | 9KH | 9KH8 | | | | | | | |
| 51 | SLUCAS | E51SLCas | 31774032 | 31774058 | + | 2037 | TCAAGCAGAGAAAGCCAGTCGG | TAAG | 7 |
| | 9KH | 9KH9 | | | | | | | |
| 51 | SLUCAS | E51SLCas | 31774043 | 31774069 | + | 2038 | AAGCCAGTCGGTAAGTTCTGTC | CAAG | 7 |
| | 9KH | 9KH10 | | | | | | | |
| 51 | SLUCAS | E51SLCas | 31774048 | 31774074 | + | 172 | AGTCGGTAAGTTCTGTCCAAGC | CCGG | 2 |
| | 9KH | 9KH11 | | | | | | | |
| 51 | SLUCAS | E51SLCas | 31774062 | 31774088 | + | 2039 | GTCCAAGCCCGGTTGAAATCTG | CCAG | 1 |
| | 9KH | 9KH12 | | | | | | | |
| 51 | SLUCAS | E51SLCas | 31774064 | 31774090 | + | 2040 | CCAAGCCCGGTTGAAATCTGCC | AGAG | 2 |
| | 9KH | 9KH13 | | | | | | | |
| 51 | SLUCAS | E51SLCas | 31774067 | 31774093 | + | 2041 | AGCCCGGTTGAAATCTGCCAGA | GCAG | 4 |
| | 9KH | 9KH14 | | | | | | | |
| 51 | SLUCAS | E51SLCas | 31774068 | 31774094 | + | 173 | GCCCGGTTGAAATCTGCCAGAG | CAGG | 5 |
| | 9KH | 9KH15 | | | | | | | |
| 51 | SLUCAS | E51SLCas | 31774084 | 31774110 | + | 2042 | CCAGAGCAGGTACCTCCAACAT | CAAG | 14 |
| | 9KH | 9KH16 | | | | | | | |
| 51 | SLUCAS | E51SLCas | 31774085 | 31774111 | + | 174 | CAGAGCAGGTACCTCCAACATC | AAGG | 13 |
| | 9KH | 9KH17 | | | | | | | |
| 51 | SLUCAS | E51SLCas | 31774088 | 31774114 | + | 2043 | AGCAGGTACCTCCAACATCAAG | GAAG | 12 |
| | 9KH | 9KH18 | | | | | | | |
| 51 | SLUCAS | E51SLCas | 31774092 | 31774118 | + | 175 | GGTACCTCCAACATCAAGGAAG | ATGG | 6 |
| | 9KH | 9KH19 | | | | | | | |
| 51 | SLUCAS | E51SLCas | 31774101 | 31774127 | + | 2044 | AACATCAAGGAAGATGGCATTT | CTAG | 32 |
| | 9KH | 9KH20 | | | | | | | |
| 51 | SLUCAS | E51SLCas | 31774106 | 31774132 | + | 176 | CAAGGAAGATGGCATTTCTAGT | TTGG | 21 |
| | 9KH | 9KH21 | | | | | | | |
| 51 | SLUCAS | E51SLCas | 31774108 | 31774134 | + | 2045 | AGGAAGATGGCATTTCTAGTTT | GGAG | 50 |
| | 9KH | 9KH22 | | | | | | | |
| 51 | SLUCAS | E51SLCas | 31774112 | 31774138 | + | 177 | AGATGGCATTTCTAGTTTGGAG | ATGG | 10 |
| | 9KH | 9KH23 | | | | | | | |
| 51 | SLUCAS | E51SLCas | 31774115 | 31774141 | + | 2046 | TGGCATTTCTAGTTTGGAGATG | GCAG | 22 |
| | 9KH | 9KH24 | | | | | | | |
| 51 | SLUCAS | E51SLCas | 31774124 | 31774150 | + | 2047 | TAGTTTGGAGATGGCAGTTTCC | TTAG | 20 |
| | 9KH | 9KH25 | | | | | | | |
| 51 | SLUCAS | E51SLCas | 31774133 | 31774159 | + | 2048 | GATGGCAGTTTCCTTAGTAACC | ACAG | 11 |
| | 9KH | 9KH26 | | | | | | | |
| 51 | SLUCAS | E51SLCas | 31774134 | 31774160 | + | 178 | ATGGCAGTTTCCTTAGTAACCA | CAGG | 14 |
| | 9KH | 9KH27 | | | | | | | |
| 51 | SLUCAS | E51SLCas | 31774146 | 31774172 | + | 2049 | TTAGTAACCACAGGTTGTGTCA | CCAG | 7 |
| | 9KH | 9KH28 | | | | | | | |
| 51 | SLUCAS | E51SLCas | 31774148 | 31774174 | + | 2050 | AGTAACCACAGGTTGTGTCACC | AGAG | 7 |
| | 9KH | 9KH29 | | | | | | | |
| 51 | SLUCAS | E51SLCas | 31774154 | 31774180 | + | 2051 | CACAGGTTGTGTCACCAGAGTA | ACAG | 14 |
| | 9KH | 9KH30 | | | | | | | |
| 51 | SLUCAS | E51SLCas | 31774160 | 31774186 | + | 2052 | TTGTGTCACCAGAGTAACAGTC | TGAG | 9 |
| | 9KH | 9KH31 | | | | | | | |
| 51 | SLUCAS | E51SLCas | 31774163 | 31774189 | + | 2053 | TGTCACCAGAGTAACAGTCTGA | GTAG | 9 |
| | 9KH | 9KH32 | | | | | | | |
| 51 | SLUCAS | E51SLCas | 31774164 | 31774190 | + | 179 | GTCACCAGAGTAACAGTCTGAG | TAGG | 14 |
| | 9KH | 9KH33 | | | | | | | |
| 51 | SLUCAS | E51SLCas | 31774166 | 31774192 | + | 2054 | CACCAGAGTAACAGTCTGAGTA | GGAG | 11 |
| | 9KH | 9KH34 | | | | | | | |
| 51 | SLUCAS | E51SLCas | 31774180 | 31774206 | + | 180 | TCTGAGTAGGAGCTAAAATATT | TTGG | 11 |
| | 9KH | 9KH35 | | | | | | | |
| 51 | SLUCAS | E51SLCas | 31774181 | 31774207 | + | 181 | CTGAGTAGGAGCTAAAATATTT | TGGG | 21 |
| | 9KH | 9KH36 | | | | | | | |
| 51 | SLUCAS | E51SLCas | 31774194 | 31774220 | + | 2055 | AAAATATTTTGGGTTTTTGCAA | AAAG | 56 |
| | 9KH | 9KH37 | | | | | | | |
| 51 | SLUCAS | E51SLCas | 31774195 | 31774221 | + | 182 | AAATATTTTGGGTTTTTGCAAA | AAGG | 61 |
| | 9KH | 9KH38 | | | | | | | |
| 51 | SLUCAS | E51SLCas | 31773927 | 31773953 | − | 2056 | GAAAAAATGATAAAAGTTGGCA | GAAG | 77 |
| | 9KH | 9KH39 | | | | | | | |
| 51 | SLUCAS | E51SLCas | 31773930 | 31773956 | − | 2057 | TGAGAAAAAATGATAAAAGTTG | GCAG | 124 |
| | 9KH | 9KH40 | | | | | | | |
| 51 | SLUCAS | E51SLCas | 31773933 | 31773959 | − | 183 | GTATGAGAAAAAATGATAAAAG | TTGG | 99 |
| | 9KH | 9KH41 | | | | | | | |
| 51 | SLUCAS | E51SLCas | 31773937 | 31773963 | − | 2058 | GAAGGTATGAGAAAAAATGATA | AAAG | 64 |
| | 9KH | 9KH42 | | | | | | | |
| 51 | SLUCAS | E51SLCas | 31773952 | 31773978 | − | 2059 | GATGATCATCAAGCAGAAGGTA | TGAG | 11 |
| | 9KH | 9KH43 | | | | | | | |
| 51 | SLUCAS | E51SLCas | 31773958 | 31773984 | − | 184 | CAACGAGATGATCATCAAGCAG | AAGG | 7 |
| | 9KH | 9KH44 | | | | | | | |
| 51 | SLUCAS | E51SLCas | 31773959 | 31773985 | − | 2060 | TCAACGAGATGATCATCAAGCA | GAAG | 3 |
| | 9KH | 9KH45 | | | | | | | |
| 51 | SLUCAS | E51SLCas | 31773962 | 31773988 | − | 2061 | ATATCAACGAGATGATCATCAA | GCAG | 7 |
| | 9KH | 9KH46 | | | | | | | |
| 51 | SLUCAS | E51SLCas | 31773965 | 31773991 | − | 2062 | AGGATATCAACGAGATGATCAT | CAAG | 7 |
| | 9KH | 9KH47 | | | | | | | |
| 51 | SLUCAS | E51SLCas | 31773977 | 31774003 | − | 2063 | TGGGTGACCTTGAGGATATCAA | CGAG | 17 |
| | 9KH | 9KH48 | | | | | | | |
| 51 | SLUCAS | E51SLCas | 31773988 | 31774014 | − | 185 | GAGGGTGATGGTGGGTGACCTT | GAGG | 22 |
| | 9KH | 9KH49 | | | | | | | |
| 51 | SLUCAS | E51SLCas | 31773989 | 31774015 | − | 2064 | AGAGGGTGATGGTGGGTGACCT | TGAG | 48 |
| | 9KH | 9KH50 | | | | | | | |
| 51 | SLUCAS | E51SLCas | 31773999 | 31774025 | − | 186 | ATAAAATCACAGAGGGTGATGG | TGGG | 27 |
| | 9KH | 9KH51 | | | | | | | |
| 51 | SLUCAS | E51SLCas | 31774000 | 31774026 | − | 187 | TATAAAATCACAGAGGGTGATG | GTGG | 23 |
| | 9KH | 9KH52 | | | | | | | |
| 51 | SLUCAS | E51SLCas | 31774003 | 31774029 | − | 188 | AGTTATAAAATCACAGAGGGTG | ATGG | 20 |
| | 9KH | 9KH53 | | | | | | | |
| 51 | SLUCAS | E51SLCas | 31774009 | 31774035 | − | 189 | TGATCAAGTTATAAAATCACAG | AGGG | 29 |
| | 9KH | 9KH54 | | | | | | | |
| 51 | SLUCAS | E51SLCas | 31774010 | 31774036 | − | 190 | TTGATCAAGTTATAAAATCACA | GAGG | 30 |
| | 9KH | 9KH55 | | | | | | | |
| 51 | SLUCAS | E51SLCas | 31774011 | 31774037 | − | 2065 | CTTGATCAAGTTATAAAATCAC | AGAG | 14 |
| | 9KH | 9KH56 | | | | | | | |
| 51 | SLUCAS | E51SLCas | 31774013 | 31774039 | − | 2066 | TGCTTGATCAAGTTATAAAATC | ACAG | 11 |
| | 9KH | 9KH57 | | | | | | | |
| 51 | SLUCAS | E51SLCas | 31774027 | 31774053 | − | 2067 | CGACTGGCTTTCTCTGCTTGAT | CAAG | 8 |
| | 9KH | 9KH58 | | | | | | | |
| 51 | SLUCAS | E51SLCas | 31774046 | 31774072 | − | 191 | GGGCTTGGACAGAACTTACCGA | CTGG | 2 |
| | 9KH | 9KH59 | | | | | | | |
| 51 | SLUCAS | E51SLCas | 31774060 | 31774086 | − | 2068 | GGCAGATTTCAACCGGGCTTGG | ACAG | 5 |
| | 9KH | 9KH60 | | | | | | | |
| 51 | SLUCAS | E51SLCas | 31774064 | 31774090 | − | 192 | CTCTGGCAGATTTCAACCGGGC | TTGG | 1 |
| | 9KH | 9KH61 | | | | | | | |
| 51 | SLUCAS | E51SLCas | 31774069 | 31774095 | − | 193 | ACCTGCTCTGGCAGATTTCAAC | CGGG | 10 |
| | 9KH | 9KH62 | | | | | | | |
| 51 | SLUCAS | E51SLCas | 31774070 | 31774096 | − | 194 | TACCTGCTCTGGCAGATTTCAA | CCGG | 10 |
| | 9KH | 9KH63 | | | | | | | |
| 51 | SLUCAS | E51SLCas | 31774081 | 31774107 | − | 2069 | GATGTTGGAGGTACCTGCTCTG | GCAG | 7 |
| | 9KH | 9KH64 | | | | | | | |
| 51 | SLUCAS | E51SLCas | 31774084 | 31774110 | − | 195 | CTTGATGTTGGAGGTACCTGCT | CTGG | 7 |
| | 9KH | 9KH65 | | | | | | | |
| 51 | SLUCAS | E51SLCas | 31774096 | 31774122 | − | 196 | AATGCCATCTTCCTTGATGTTG | GAGG | 22 |
| | 9KH | 9KH66 | | | | | | | |
| 51 | SLUCAS | E51SLCas | 31774097 | 31774123 | − | 2070 | AAATGCCATCTTCCTTGATGTT | GGAG | 19 |
| | 9KH | 9KH67 | | | | | | | |
| 51 | SLUCAS | E51SLCas | 31774099 | 31774125 | − | 19 | AGAAATGCCATCTTCCTTGATG | TTGG | 26 |
| | 9KH | 9KH68 | | | | | | | |
| 51 | SLUCAS | E51SLCas | 31774123 | 31774149 | − | 2071 | TAAGGAAACTGCCATCTCCAAA | CTAG | 41 |
| | 9KH | 9KH69 | | | | | | | |
| 51 | SLUCAS | E51SLCas | 31774144 | 31774170 | − | 198 | GGTGACACAACCTGTGGTTACT | AAGG | 6 |
| | 9KH | 9KH70 | | | | | | | |
| 51 | SLUCAS | E51SLCas | 31774145 | 31774171 | − | 2072 | TGGTGACACAACCTGTGGTTAC | TAAG | 10 |
| | 9KH | 9KH71 | | | | | | | |
| 51 | SLUCAS | E51SLCas | 31774153 | 31774179 | − | 199 | TGTTACTCTGGTGACACAACCT | GTGG | 22 |
| | 9KH | 9KH72 | | | | | | | |
| 51 | SLUCAS | E51SLCas | 31774168 | 31774194 | − | 200 | AGCTCCTACTCAGACTGTTACT | CTGG | 2 |
| | 9KH | 9KH73 | | | | | | | |
| 51 | SLUCAS | E51SLCas | 31774181 | 31774207 | − | 2073 | CCCAAAATATTTTAGCTCCTAC | TCAG | 10 |
| | 9KH | 9KH74 | | | | | | | |
| 51 | SLUCAS | E51SLCas | 31774192 | 31774218 | − | 2074 | TTTTGCAAAAACCCAAAATATT | TTAG | 43 |
| | 9KH | 9KH75 | | | | | | | |
| 53 | SACAS9 | E53SaCas | 31679352 | 31679380 | + | 1056 | AAAAGGTATCTTTGATACTAAC | CTTGGT | 45 |
| | KKH | 9KKH1 | | | | | | | |
| 53 | SACAS9 | E53SaCas | 31679361 | 31679389 | + | 1057 | CTTTGATACTAACCTTGGTTTC | TGTGAT | 20 |
| | KKH | 9KKH2 | | | | | | | |
| 53 | SACAS9 | E53SaCas | 31679373 | 31679401 | + | 25 | CCTTGGTTTCTGTGATTTTCTT | TTGGAT | 122 |
| | KKH | 9KKH3 | | | | | | | |
| 53 | SACAS9 | E53SaCas | 31679477 | 31679505 | + | 26 | TCCTTAGCTTCCAGCCATTGTG | TTGAAT | 42 |
| | KKH | 9KKH4 | | | | | | | |
| 53 | SACAS9 | E53SaCas | 31679510 | 31679538 | + | 1058 | AACATTTCATTCAACTGTTGCC | TCCGGT | 46 |
| | KKH | 9KKH5 | | | | | | | |
| 53 | SACAS9 | E53SaCas | 31679519 | 31679547 | + | 1059 | TTCAACTGTTGCCTCCGGTTCT | GAAGGT | 9 |
| | KKH | 9KKH6 | | | | | | | |
| 53 | SACAS9 | E53SaCas | 31679543 | 31679571 | + | 1060 | AGGTGTTCTTGTACTTCATCCC | ACTGAT | 16 |
| | KKH | 9KKH7 | | | | | | | |
| 53 | SACAS9 | E53SaCas | 31679550 | 31679578 | + | 27 | CTTGTACTTCATCCCACTGATT | CTGAAT | 58 |
| | KKH | 9KKH8 | | | | | | | |
| 53 | SACAS9 | E53SaCas | 31679565 | 31679593 | + | 28 | ACTGATTCTGAATTCTTTCAAC | TAGAAT | 78 |
| | KKH | 9KKH9 | | | | | | | |
| 53 | SACAS9 | E53SaCas | 31679577 | 31679605 | + | 1061 | TTCTTTCAACTAGAATAAAAGG | AAAAAT | 86 |
| | KKH | 9KKH10 | | | | | | | |
| 53 | SACAS9 | E53SaCas | 31679581 | 31679609 | + | 1062 | TTCAACTAGAATAAAAGGAAAA | ATAAAT | 420 |
| | KKH | 9KKH11 | | | | | | | |
| 53 | SACAS9 | E53SaCas | 31679588 | 31679616 | + | 1063 | AGAATAAAAGGAAAAATAAATA | TATAGT | 5266 |
| | KKH | 9KKH12 | | | | | | | |
| 53 | SACAS9 | E53SaCas | 31679346 | 31679374 | − | 1064 | GTTAGTATCAAAGATACCTTTT | TAAAAT | 31 |
| | KKH | 9KKH13 | | | | | | | |
| 53 | SACAS9 | E53SaCas | 31679359 | 31679387 | − | 1065 | CACAGAAACCAAGGTTAGTATC | AAAGAT | 33 |
| | KKH | 9KKH14 | | | | | | | |
| 53 | SACAS9 | E53SaCas | 31679368 | 31679396 | − | 1066 | AAAGAAAATCACAGAAACCAAG | GTTAGT | 456 |
| | KKH | 9KKH15 | | | | | | | |
| 53 | SACAS9 | E53SaCas | 31679372 | 31679400 | − | 1067 | TCCAAAAGAAAATCACAGAAAC | CAAGGT | 24 |
| | KKH | 9KKH16 | | | | | | | |
| 53 | SACAS9 | E53SaCas | 31679387 | 31679415 | − | 1068 | ATACAGTAGATGCAATCCAAAA | GAAAAT | 49 |
| | KKH | 9KKH17 | | | | | | | |
| 53 | SACAS9 | E53SaCas | 31679399 | 31679427 | − | 1069 | AGGAGGGTCCCTATACAGTAGA | TGCAAT | 13 |
| | KKH | 9KKH18 | | | | | | | |
| 53 | SACAS9 | E53SaCas | 31679404 | 31679432 | − | 1070 | ATGGAAGGAGGGTCCCTATACA | GTAGAT | 20 |
| | KKH | 9KKH19 | | | | | | | |
| 53 | SACAS9 | E53SaCas | 31679408 | 31679436 | − | 1071 | AGTCATGGAAGGAGGGTCCCTA | TACAGT | 27 |
| | KKH | 9KKH20 | | | | | | | |
| 53 | SACAS9 | E53SaCas | 31679419 | 31679447 | − | 29 | AGCCAAGCTTGAGTCATGGAAG | GAGGGT | 36 |
| | KKH | 9KKH21 | | | | | | | |
| 53 | SACAS9 | E53SaCas | 31679433 | 31679461 | − | 30 | TTAGGACAGGCCAGAGCCAAGC | TTGAGT | 30 |
| | KKH | 9KKH22 | | | | | | | |
| 53 | SACAS9 | E53SaCas | 31679462 | 31679490 | − | 1072 | TGGAAGCTAAGGAAGAAGCTGA | GCAGGT | 103 |
| | KKH | 9KKH23 | | | | | | | |
| 53 | SACAS9 | E53SaCas | 31679493 | 31679521 | − | 1073 | AATGAAATGTTAAAGGATTCAA | CACAAT | 200 |
| | KKH | 9KKH24 | | | | | | | |
| 53 | SACAS9 | E53SaCas | 31679503 | 31679531 | − | 31 | GCAACAGTTGAATGAAATGTTA | AAGGAT | 155 |
| | KKH | 9KKH25 | | | | | | | |
| 53 | SACAS9 | E53SaCas | 31679513 | 31679541 | − | 1074 | AGAACCGGAGGCAACAGTTGAA | TGAAAT | 11 |
| | KKH | 9KKH26 | | | | | | | |
| 53 | SACAS9 | E53SaCas | 31679518 | 31679546 | − | 32 | CCTTCAGAACCGGAGGCAACAG | TTGAAT | 8 |
| | KKH | 9KKH27 | | | | | | | |
| 53 | SACAS9 | E53SaCas | 31679523 | 31679551 | − | 1075 | GAACACCTTCAGAACCGGAGGC | AACAGT | 10 |
| | KKH | 9KKH28 | | | | | | | |
| 53 | SACAS9 | E53SaCas | 31679555 | 31679583 | − | 1076 | AAAGAATTCAGAATCAGTGGGA | TGAAGT | 106 |
| | KKH | 9KKH29 | | | | | | | |
| 53 | SACAS9 | E53SaCas | 31679560 | 31679588 | − | 33 | AGTTGAAAGAATTCAGAATCAG | TGGGAT | 110 |
| | KKH | 9KKH30 | | | | | | | |
| 53 | SACAS9 | E53SaCas | 31679565 | 31679593 | − | 1077 | ATTCTAGTTGAAAGAATTCAGA | ATCAGT | 134 |
| | KKH | 9KKH31 | | | | | | | |
| 53 | SACAS9 | E53SaCas | 31679569 | 31679597 | − | 34 | TTTTATTCTAGTTGAAAGAATT | CAGAAT | 399 |
| | KKH | 9KKH32 | | | | | | | |
| 53 | SACAS9 | E53SaCas | 31679576 | 31679604 | − | 35 | TTTTTCCTTTTATTCTAGTTGA | AAGAAT | 475 |
| | KKH | 9KKH33 | | | | | | | |
| 53 | SACAS9 | E53SaCas | 31679585 | 31679613 | − | 1078 | ATATATTTATTTTTCCTTTTAT | TCTAGT | 2133 |
| | KKH | 9KKH34 | | | | | | | |
| 53 | SLUCAS | E53SLCas | 31679353 | 31679379 | + | 201 | AAAGGTATCTTTGATACTAACC | TTGG | 13 |
| | 9KH | 9KH1 | | | | | | | |
| 53 | SLUCAS | E53SLCas | 31679373 | 31679399 | + | 202 | CCTTGGTTTCTGTGATTTTCTT | TTGG | 51 |
| | 9KH | 9KH2 | | | | | | | |
| 53 | SLUCAS | E53SLCas | 31679391 | 31679417 | + | 2075 | TCTTTTGGATTGCATCTACTGT | ATAG | 18 |
| | 9KH | 9KH3 | | | | | | | |
| 53 | SLUCAS | E53SLCas | 31679392 | 31679418 | + | 203 | CTTTTGGATTGCATCTACTGTA | TAGG | 12 |
| | 9KH | 9KH4 | | | | | | | |
| 53 | SLUCAS | E53SLCas | 31679393 | 31679419 | + | 204 | TTTTGGATTGCATCTACTGTAT | AGGG | 15 |
| | 9KH | 9KH5 | | | | | | | |
| 53 | SLUCAS | E53SLCas | 31679414 | 31679440 | + | 2076 | TAGGGACCCTCCTTCCATGACT | CAAG | 11 |
| | 9KH | 9KH6 | | | | | | | |
| 53 | SLUCAS | E53SLCas | 31679419 | 31679445 | + | 205 | ACCCTCCTTCCATGACTCAAGC | TTGG | 11 |
| | 9KH | 9KH7 | | | | | | | |
| 53 | SLUCAS | E53SLCas | 31679425 | 31679451 | + | 206 | CTTCCATGACTCAAGCTTGGCT | CTGG | 10 |
| | 9KH | 9KH8 | | | | | | | |
| 53 | SLUCAS | E53SLCas | 31679436 | 31679462 | + | 2077 | CAAGCTTGGCTCTGGCCTGTCC | TAAG | 16 |
| | 9KH | 9KH9 | | | | | | | |
| 53 | SLUCAS | E53SLCas | 31679446 | 31679472 | + | 2078 | TCTGGCCTGTCCTAAGACCTGC | TCAG | 18 |
| | 9KH | 9KH10 | | | | | | | |
| 53 | SLUCAS | E53SLCas | 31679458 | 31679484 | + | 2079 | TAAGACCTGCTCAGCTTCTTCC | TTAG | 16 |
| | 9KH | 9KH11 | | | | | | | |
| 53 | SLUCAS | E53SLCas | 31679465 | 31679491 | + | 2080 | TGCTCAGCTTCTTCCTTAGCTT | CCAG | 24 |
| | 9KH | 9KH12 | | | | | | | |
| 53 | SLUCAS | E53SLCas | 31679511 | 31679537 | + | 207 | ACATTTCATTCAACTGTTGCCT | CCGG | 15 |
| | 9KH | 9KH13 | | | | | | | |
| 53 | SLUCAS | E53SLCas | 31679519 | 31679545 | + | 2081 | TTCAACTGTTGCCTCCGGTTCT | GAAG | 6 |
| | 9KH | 9KH14 | | | | | | | |
| 53 | SLUCAS | E53SLCas | 31679520 | 31679546 | + | 208 | TCAACTGTTGCCTCCGGTTCTG | AAGG | 4 |
| | 9KH | 9KH15 | | | | | | | |
| 53 | SLUCAS | E53SLCas | 31679564 | 31679590 | + | 2082 | CACTGATTCTGAATTCTTTCAA | CTAG | 32 |
| | 9KH | 9KH16 | | | | | | | |
| 53 | SLUCAS | E53SLCas | 31679572 | 31679598 | + | 2083 | CTGAATTCTTTCAACTAGAATA | AAAG | 71 |
| | 9KH | 9KH17 | | | | | | | |
| 53 | SLUCAS | E53SLCas | 31679573 | 31679599 | + | 209 | TGAATTCTTTCAACTAGAATAA | AAGG | 48 |
| | 9KH | 9KH18 | | | | | | | |
| 53 | SLUCAS | E53SLCas | 31679589 | 31679615 | + | 2084 | GAATAAAAGGAAAAATAAATAT | ATAG | 574 |
| | 9KH | 9KH19 | | | | | | | |
| 53 | SLUCAS | E53SLCas | 31679592 | 31679618 | + | 2085 | TAAAAGGAAAAATAAATATATA | GTAG | 765 |
| | 9KH | 9KH20 | | | | | | | |
| 53 | SLUCAS | E53SLCas | 31679361 | 31679387 | − | 2086 | CACAGAAACCAAGGTTAGTATC | AAAG | 6 |
| | 9KH | 9KH21 | | | | | | | |
| 53 | SLUCAS | E53SLCas | 31679369 | 31679395 | − | 2087 | AAGAAAATCACAGAAACCAAGG | TTAG | 88 |
| | 9KH | 9KH22 | | | | | | | |
| 53 | SLUCAS | E53SLCas | 31679373 | 31679399 | − | 210 | CCAAAAGAAAATCACAGAAACC | AAGG | 52 |
| | 9KH | 9KH23 | | | | | | | |
| 53 | SLUCAS | E53SLCas | 31679374 | 31679400 | − | 2088 | TCCAAAAGAAAATCACAGAAAC | CAAG | 75 |
| | 9KH | 9KH24 | | | | | | | |
| 53 | SLUCAS | E53SLCas | 31679382 | 31679408 | − | 2089 | AGATGCAATCCAAAAGAAAATC | ACAG | 89 |
| | 9KH | 9KH25 | | | | | | | |
| 53 | SLUCAS | E53SLCas | 31679392 | 31679418 | − | 2090 | CCTATACAGTAGATGCAATCCA | AAAG | 4 |
| | 9KH | 9KH26 | | | | | | | |
| 53 | SLUCAS | E53SLCas | 31679406 | 31679432 | − | 2091 | ATGGAAGGAGGGTCCCTATACA | GTAG | 8 |
| | 9KH | 9KH27 | | | | | | | |
| 53 | SLUCAS | E53SLCas | 31679409 | 31679435 | − | 2092 | GTCATGGAAGGAGGGTCCCTAT | ACAG | 12 |
| | 9KH | 9KH28 | | | | | | | |
| 53 | SLUCAS | E53SLCas | 31679420 | 31679446 | − | 211 | GCCAAGCTTGAGTCATGGAAGG | AGGG | 10 |
| | 9KH | 9KH29 | | | | | | | |
| 53 | SLUCAS | E53SLCas | 31679421 | 31679447 | − | 212 | AGCCAAGCTTGAGTCATGGAAG | GAGG | 21 |
| | 9KH | 9KH30 | | | | | | | |
| 53 | SLUCAS | E53SLCas | 31679422 | 31679448 | − | 2093 | GAGCCAAGCTTGAGTCATGGAA | GGAG | 14 |
| | 9KH | 9KH31 | | | | | | | |
| 53 | SLUCAS | E53SLCas | 31679424 | 31679450 | − | 213 | CAGAGCCAAGCTTGAGTCATGG | AAGG | 18 |
| | 9KH | 9KH32 | | | | | | | |
| 53 | SLUCAS | E53SLCas | 31679425 | 31679451 | − | 2094 | CCAGAGCCAAGCTTGAGTCATG | GAAG | 18 |
| | 9KH | 9KH33 | | | | | | | |
| 53 | SLUCAS | E53SLCas | 31679428 | 31679454 | − | 214 | AGGCCAGAGCCAAGCTTGAGTC | ATGG | 23 |
| | 9KH | 9KH34 | | | | | | | |
| 53 | SLUCAS | E53SLCas | 31679434 | 31679460 | − | 2095 | TAGGACAGGCCAGAGCCAAGCT | TGAG | 17 |
| | 9KH | 9KH35 | | | | | | | |
| 53 | SLUCAS | E53SLCas | 31679440 | 31679466 | − | 2096 | AGGTCTTAGGACAGGCCAGAGC | CAAG | 23 |
| | 9KH | 9KH36 | | | | | | | |
| 53 | SLUCAS | E53SLCas | 31679445 | 31679471 | − | 2097 | TGAGCAGGTCTTAGGACAGGCC | AGAG | 27 |
| | 9KH | 9KH37 | | | | | | | |
| 53 | SLUCAS | E53SLCas | 31679447 | 31679473 | − | 2098 | GCTGAGCAGGTCTTAGGACAGG | CCAG | 35 |
| | 9KH | 9KH38 | | | | | | | |
| 53 | SLUCAS | E53SLCas | 31679451 | 31679477 | − | 215 | AGAAGCTGAGCAGGTCTTAGGA | CAGG | 44 |
| | 9KH | 9KH39 | | | | | | | |
| 53 | SLUCAS | E53SLCas | 31679452 | 31679478 | − | 2099 | AAGAAGCTGAGCAGGTCTTAGG | ACAG | 16 |
| | 9KH | 9KH40 | | | | | | | |
| 53 | SLUCAS | E53SLCas | 31679456 | 31679482 | − | 216 | AAGGAAGAAGCTGAGCAGGTCT | TAGG | 53 |
| | 9KH | 9KH41 | | | | | | | |
| 53 | SLUCAS | E53SLCas | 31679457 | 31679483 | − | 2100 | TAAGGAAGAAGCTGAGCAGGTC | TTAG | 25 |
| | 9KH | 9KH42 | | | | | | | |
| 53 | SLUCAS | E53SLCas | 31679463 | 31679489 | − | 217 | GGAAGCTAAGGAAGAAGCTGAG | CAGG | 70 |
| | 9KH | 9KH43 | | | | | | | |
| 53 | SLUCAS | E53SLCas | 31679464 | 31679490 | − | 2101 | TGGAAGCTAAGGAAGAAGCTGA | GCAG | 52 |
| | 9KH | 9KH44 | | | | | | | |
| 53 | SLUCAS | E53SLCas | 31679467 | 31679493 | − | 2102 | GGCTGGAAGCTAAGGAAGAAGC | TGAG | 41 |
| | 9KH | 9KH45 | | | | | | | |
| 53 | SLUCAS | E53SLCas | 31679472 | 31679498 | − | 2103 | ACAATGGCTGGAAGCTAAGGAA | GAAG | 27 |
| | 9KH | 9KH46 | | | | | | | |
| 53 | SLUCAS | E53SLCas | 31679475 | 31679501 | − | 2104 | AACACAATGGCTGGAAGCTAAG | GAAG | 18 |
| | 9KH | 9KH47 | | | | | | | |
| 53 | SLUCAS | E53SLCas | 31679478 | 31679504 | − | 218 | TTCAACACAATGGCTGGAAGCT | AAGG | 87 |
| | 9KH | 9KH48 | | | | | | | |
| 53 | SLUCAS | E53SLCas | 31679479 | 31679505 | − | 2105 | ATTCAACACAATGGCTGGAAGC | TAAG | 24 |
| | 9KH | 9KH49 | | | | | | | |
| 53 | SLUCAS | E53SLCas | 31679484 | 31679510 | − | 2106 | AAAGGATTCAACACAATGGCTG | GAAG | 12 |
| | 9KH | 9KH50 | | | | | | | |
| 53 | SLUCAS | E53SLCas | 31679487 | 31679513 | − | 219 | GTTAAAGGATTCAACACAATGG | CTGG | 12 |
| | 9KH | 9KH51 | | | | | | | |
| 53 | SLUCAS | E53SLCas | 31679491 | 31679517 | − | 220 | AAATGTTAAAGGATTCAACACA | ATGG | 37 |
| | 9KH | 9KH52 | | | | | | | |
| 53 | SLUCAS | E53SLCas | 31679505 | 31679531 | − | 221 | GCAACAGTTGAATGAAATGTTA | AAGG | 28 |
| | 9KH | 9KH53 | | | | | | | |
| 53 | SLUCAS | E53SLCas | 31679506 | 31679532 | − | 2107 | GGCAACAGTTGAATGAAATGTT | AAAG | 25 |
| | 9KH | 9KH54 | | | | | | | |
| 53 | SLUCAS | E53SLCas | 31679524 | 31679550 | − | 2108 | AACACCTTCAGAACCGGAGGCA | ACAG | 7 |
| | 9KH | 9KH55 | | | | | | | |
| 53 | SLUCAS | E53SLCas | 31679530 | 31679556 | − | 222 | TACAAGAACACCTTCAGAACCG | GAGG | 10 |
| | 9KH | 9KH56 | | | | | | | |
| 53 | SLUCAS | E53SLCas | 31679531 | 31679557 | − | 2109 | GTACAAGAACACCTTCAGAACC | GGAG | 10 |
| | 9KH | 9KH57 | | | | | | | |
| 53 | SLUCAS | E53SLCas | 31679533 | 31679559 | − | 223 | AAGTACAAGAACACCTTCAGAA | CCGG | 17 |
| | 9KH | 9KH58 | | | | | | | |
| 53 | SLUCAS | E53SLCas | 31679539 | 31679565 | − | 2110 | GGGATGAAGTACAAGAACACCT | TCAG | 18 |
| | 9KH | 9KH59 | | | | | | | |
| 53 | SLUCAS | E53SLCas | 31679550 | 31679576 | − | 2111 | TCAGAATCAGTGGGATGAAGTA | CAAG | 27 |
| | 9KH | 9KH60 | | | | | | | |
| 53 | SLUCAS | E53SLCas | 31679556 | 31679582 | − | 2112 | AAGAATTCAGAATCAGTGGGAT | GAAG | 27 |
| | 9KH | 9KH61 | | | | | | | |
| 53 | SLUCAS | E53SLCas | 31679562 | 31679588 | − | 224 | AGTTGAAAGAATTCAGAATCAG | TGGG | 95 |
| | 9KH | 9KH62 | | | | | | | |
| 53 | SLUCAS | E53SLCas | 31679563 | 31679589 | − | 225 | TAGTTGAAAGAATTCAGAATCA | GTGG | 33 |
| | 9KH | 9KH63 | | | | | | | |
| 53 | SLUCAS | E53SLCas | 31679566 | 31679592 | − | 2113 | TTCTAGTTGAAAGAATTCAGAA | TCAG | 43 |
| | 9KH | 9KH64 | | | | | | | |
| 53 | SLUCAS | E53SLCas | 31679572 | 31679598 | − | 2114 | CTTTTATTCTAGTTGAAAGAAT | TCAG | 61 |
| | 9KH | 9KH65 | | | | | | | |
| 53 | SLUCAS | E53SLCas | 31679579 | 31679605 | − | 2115 | ATTTTTCCTTTTATTCTAGTTG | AAAG | 122 |
| | 9KH | 9KH66 | | | | | | | |
| 53 | SLUCAS | E53SLCas | 31679586 | 31679612 | − | 2116 | TATATTTATTTTTCCTTTTATT | CTAG | 978 |
| | 9KH | 9KH67 | | | | | | | |

In some embodiments, the payload comprises a single nucleic acid molecule comprising: i) a nucleic acid encoding Staphylococcus aureus Cas9 (SaCas9) or Staphylococcus lugdunensis (SluCas9) and at least one, at least two, or at least three guide RNAs; or ii) a nucleic acid encoding Staphylococcus aureus Cas9 (SaCas9) or Staphylococcus lugdunensis (SluCas9) and from one to n guide RNAs, wherein n is no more than the maximum number of guide RNAs that can be expressed from said nucleic acid; or iii) a nucleic acid encoding Staphylococcus aureus Cas9 (SaCas9) or Staphylococcus lugdunensis (SluCas9) and one to three guide RNAs.

In some embodiments, the payload comprises at least two nucleic acid molecules comprising a first nucleic acid encoding Staphylococcus aureus Cas9 (SaCas9) or Staphylococcus lugdunensis (SluCas9); and a second nucleic acid that does not encode a SaCas9 or SluCas9 and encodes any one of i) at least one, at least two, at least three, at least four, at least five, or at least six guide RNAs; or ii) from one to n guide RNAs, wherein n is no more than the maximum number of guide RNAs that can be expressed from said nucleic acid; or iii) from one to six guide RNAs.

In some embodiments, the payload comprises at least two nucleic acid molecules comprising a first nucleic acid encoding Staphylococcus aureus Cas9 (SaCas9) or Staphylococcus lugdunensis (SluCas9) and i) at least one, at least two, or at least three guide RNAs; or ii) from one to n guide RNAs, wherein n is no more than the maximum number of guide RNAs that can be expressed from said nucleic acid; or iii) one to three guide RNAs; and a second nucleic acid that does not encode a SaCas9 or SluCas9, optionally wherein the second nucleic acid comprises any one of i) at least one, at least two, at least three, at least four, at least five, or at least six guide RNAs; or ii) from one to n guide RNAs, wherein n is no more than the maximum number of guide RNAs that can be expressed from said nucleic acid; or iii) from one to six guide RNAs.

In some embodiments, the payload comprises at least two nucleic acid molecules comprising a first nucleic acid encoding Staphylococcus aureus Cas9 (SaCas9) or Staphylococcus lugdunensis (SluCas9) and at least one, at least two, or at least three guide RNAs; and a second nucleic acid that does not encode a SaCas9 or SluCas9 and encodes from one to six guide RNAs.

In some embodiments, the payload comprises at least two nucleic acid molecules comprising a first nucleic acid encoding Staphylococcus aureus Cas9 (SaCas9) or Staphylococcus lugdunensis (SluCas9) and at least two guide RNAs, wherein at least one guide RNA binds upstream of sequence to be excised and at least one guide RNA binds downstream of sequence to be excised; and a second nucleic acid that does not encode a SaCas9 or SluCas9 and encodes at least one additional copy of the guide RNAs encoded in the first nucleic acid. In some embodiments, the guide RNA excises a portion of a DMD gene, optionally an exon, intron, or exon/intron junction.

In some embodiments, a payload is provided comprising, consisting of, or consisting essentially of at least two nucleic acid molecules comprising a first nucleic acid encoding Staphylococcus aureus Cas9 (SaCas9) or Staphylococcus lugdunensis (SluCas9) and a first and a second guide RNA that function to excise a portion of a DMD gene; and a second nucleic acid encoding at least 2 or at least 3 copies of the first guide RNA and at least 2 or at least 3 copies of the second guide RNA.

In some embodiments, a payload is provided comprising, consisting of, or consisting essentially of one or more nucleic acid molecules encoding an endonuclease and a pair of guide RNAs, wherein each guide RNA targets a different sequence in a DMD gene, wherein the endonuclease and pair of guide RNAs are capable of excising a target sequence in DNA that is between 5-250 nucleotides in length. In some embodiments, the endonuclease is a class 2, type II Cas endonuclease. In some embodiments, the class 2, type II Cas endonuclease is SpCas9, SaCas9, or SluCas9. In some embodiments, the endonuclease is not a class 2, type V Cas endonuclease. In some embodiments, the excised target sequence comprises a splice acceptor site or a splice donor site. In some embodiments, the excised target sequence comprises a premature stop codon in the DMD gene. In some embodiments, the excised target sequence does not comprise an entire exon of the DMD gene. In some embodiments, any of the methods and/or ribonucleoprotein complexes disclosed herein do not destroy/specifically alter the sequence of a splice acceptor site, splice donor site, or premature stop codon site.

### III. Methods

Disclosed herein are methods comprising (a) administering to a subject in need thereof an agent that blocks LDL binding to a low-density lipoprotein receptor (LDLR) and/or prevents LNP uptake into liver cells or tissue and concurrently or sequentially (b) administering an LNP or lipoplex, comprising a payload to the subject, wherein the agent reduces off-target delivery of the LNP or lipoplex to the liver.

Disclosed herein are methods of increasing the percentage of payload delivered to anon-liver target in a subject, comprising (a) a pre-conditioning step comprising administering to the subject a composition comprising an agent that blocks LDL binding to a low-density lipoprotein receptor (LDLR) in the liver, and then (b) administering an LNP, or lipoplex, and a payload to the subject.

Disclosed herein are methods of decreasing liver tropism of a payload administered in an LNP or lipoplex in a subject comprising (a) administering to the subject a composition comprising i) an agent that blocks low-density lipoprotein (LDL) binding to a low-density lipoprotein receptor (LDLR) and/or prevents LNP uptake into liver cells or tissue and ii) a delivery molecule, wherein the delivery molecule delivers the agent to the liver, and then (b) administering an LNP or lipoplex and a payload to the subject.

In some embodiments, administration of the composition comprising the agent that blocks LDL binding to LDLR (e.g., an ASO) temporarily blocks the delivery molecule (e.g., LNP) from interacting with the LDLR in the liver. In some embodiments, administration of the composition comprising the agent that blocks LDL binding to LDLR (e.g., ASO) increases the percentage of the agent delivered to the non-liver target.

In some embodiments, the methods comprise administering an agent that blocks LDL binding to an LDL receptor (LDLR) and a delivery molecule that delivers the agent to the liver and induces long-term blocking of LDL binding to LDLR, e.g., longer than about 3 weeks. In some embodiments, the blocking of LDL binding to LDL receptors in the liver is not temporary. In some embodiments, the blocking of LDL binding to LDL receptors in the liver is temporary.

In some embodiments, administering to a subject in need thereof a composition for blocking LDL binding to LDL receptors and/or for preventing LNP uptake into liver cells or tissue occurs prior to administering the LNP or lipoplex, and payload, for example at least about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days prior to administering the LNP or lipoplex and payload. In some embodiments, the disclosure provides for a method of administering to a subject in need thereof a composition for blocking LDL binding to LDL receptors and/or for preventing LNP uptake into liver cells or tissue occurs prior to administering the LNP or lipoplex and payload, for example, at 1-2, 1-3, 2-5, 4-7, 6-9, 8-11, 10-13 or 12-15 days prior to administering the LNP or lipoplex. In some embodiments, the disclosure provides for a method of administering to a subject in need thereof a composition for blocking LDL binding to LDL receptors and/or for preventing LNP uptake into liver cells or tissue occurs prior to administering the LNP or lipoplex and payload, for example, at least 2-21 days, at least 2-3 days, at least 3-5 days, at least 7-14 days, at least 10-14 days, or at least 14-21 days prior to administering the LNP or lipoplex and payload. In some embodiments, the subject is administered more than one dose (e.g., 2, 3 or 4) of the agent that blocks LDL binding to the LDLR and/or prevents LNP uptake into liver cells or tissue prior to being administered the payload. In some embodiments, administration of the composition for blocking LDL binding to LDL receptors and/or for preventing LNP uptake into liver cells or tissue immediately precedes the administration of the LNP or lipoplex and payload. In some embodiments, the composition for blocking LDL binding to LDL receptors and/or for preventing LNP uptake into liver cells or tissue is co-administered with the LNP or lipoplex and payload. In some embodiments, the composition comprising an agent that blocks LDL binding to an LDL receptor (LDLR) and/or prevents LNP uptake into liver cells or tissue and the LNP or lipoplex, and payload are administered concurrently. In some embodiments, the composition comprising an agent that blocks LDL binding to an LDL receptor (LDLR) and/or for preventing LNP uptake into liver cells or tissue and the LNP or lipoplex and payload are administered sequentially.

In some embodiments, the composition that blocks LDL binding to an LDL receptor and/or prevents LNP uptake into liver cells or tissue (LDLR) comprises an RNAi (e.g., siRNA or ASO). In some embodiments, the composition that blocks LDL binding to an LDL receptor (LDLR) and/or prevents LNP uptake into liver cells or tissue comprises an antisense oligonucleotide (ASO). In some embodiments, the composition that blocks LDL binding to an LDL receptor (LDLR) and/or prevents LNP uptake into liver cells or tissue comprises an siRNA. In some embodiments, the composition that blocks LDL binding to an LDL receptor (LDLR) and/or prevents LNP uptake into liver cells or tissue comprises a non-RNAi, such as a small molecule, antibody, soluble receptor, or non-antibody inhibitor.

The method results in the non-liver payload being more effectively routed to the intended target by way of the decreased tropism to the liver.

In some embodiments, systemically or locally delivered agents are delivered to induce a temporary gene expression knockdown effect of about 50%, about 60%, about 70%, or about 80% from about 24 hours to about 48 hours to greater than or about 10 days. After the agent is administered and temporary knockdown of the LDLR is induced, non-liver payload-based therapy can be administered.

In some embodiments, the method includes administering an agent that is capable of temporarily blocking LDL binding to LDLR receptors and/or preventing LNP uptake into liver cells or tissue, including for about 48 hours to 3 weeks. In some embodiments, the RNAi molecules (e.g., any of the siRNAs or ASOs disclosed herein) and/or compositions are capable of blocking LDL binding to LDLR and/or preventing LNP uptake into liver cells or tissue for about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days. In some embodiments, the RNAi molecules (e.g., any of the siRNAs or ASOs disclosed herein) and/or compositions are capable of blocking LDL binding to LDLR and/or preventing LNP uptake into liver cells or tissue for about 7 days, about 8 days, about 9 days, about 10 days, about 11 days, about 12 days, about 13 days, about 14 days. In some embodiments, the agent, e.g., RNAi molecule and/or composition disclosed herein is capable of blocking LDL binding to LDL receptors and/or preventing LNP uptake into liver cells or tissue in the liver for 1-28 days, 2-28 days, 3-28 days, 7-28 days, 10-28 days, 14-28 days, 21-28 days, 1-21 days, 2-21 days, 3-21 days, 7-21 days, 10-21 days, 14-21 days, 1-14 days, 2-14 days, 3-14 days, 7-14 days, 1-10 days, 2-10 days, 3-10 days, 7-10 days, 1-7 days, 2-7 days, 3-7 days, 1-4 days, 1-3 days, or about 24 hours to about 48 hours.

In some embodiments, the agent (e.g., RNAi molecule and/or composition disclosed herein) knocks down LDLR and comprises a ribonucleotide sequence at least 80% identical to a ribonucleotide sequence from the LDLR. Preferably, the agent is at least 90%, 95%, 96%, 97%, 98%, 99% or 100% identical to the ribonucleotide sequence of the target, or complement thereof. In some embodiments, an agent will be 100% identical to the nucleotide sequence or complement thereof of a target sequence. However, RNAi agents with insertions, deletions or single point mutations relative to a target may also be effective. Tools to assist siRNA design are readily available to the public and are known in the art.

In some embodiments, the subject is administered an amount of the agent that knocks down the levels of LDLR in the liver by at least 10%, 20%, 30%, 40%, 50%, 60%, 70%, 80%, 90%, 95%, or 99% as compared to a control subject not administered the agent. In some embodiments, the subject is administered an amount of the agent that knocks down the levels of LDLR in the liver by 80-100%, 80-95%, 10-90%, 10-70%, 10-50%, 10-30%, 30-90%, 30-70%, 30-50%, 50-90%, 50-70%, or 70-90% as compared to a control subject not administered the agent.

In some embodiments, the subject is administered an amount of the agent that knocks down the levels of LDLR in the liver for 1-28 days, 2-28 days, 3-28 days, 7-28 days, 10-28 days, 14-28 days, 21-28 days, 1-21 days, 2-21 days, 3-21 days, 7-21 days, 10-21 days, 14-21 days, 1-14 days, 2-14 days, 3-14 days, 7-14 days, 1-10 days, 2-10 days, 3-10 days, 7-10 days, 1-7 days, 2-7 days, 3-7 days, 1-4 days, 1-3 days, or about 24 hours to about 48 hours.

In some embodiments, the subject is administered an amount of an ASO or siRNA that knocks down the levels of LDLR in the liver by at least 10%, 20%, 30%, 40%, 50%, 60%, 70%, 80%, 90%, 95%, or 99% as compared to a control subject not administered the ASO or siRNA. In some embodiments, the subject is administered an amount of an ASO that knocks down the levels of LDLR in the liver by 80-100%, 80-95%, 10-90%, 10-70%, 10-50%, 10-30%, 30-90%, 30-70%, 30-50%, 50-90%, 50-70%, or 70-90% as compared to a control subject not administered the ASO or siRNA.

In some embodiments, the subject is administered an amount of the ASO or siRNA that knocks down the levels of LDLR in the liver for 1-28 days, 2-28 days, 3-28 days, 7-28 days, 10-28 days, 14-28 days, 21-28 days, 1-21 days, 2-21 days, 3-21 days, 7-21 days, 10-21 days, 14-21 days, 1-14 days, 2-14 days, 3-14 days, 7-14 days, 1-10 days, 2-10 days, 3-10 days, 7-10 days, 1-7 days, 2-7 days, 3-7 days, 1-4 days, 1-3 days, or about 24 hours to about 48 hours. In some embodiments, the subject is administered an amount of the ASO or siRNA that knocks down the levels of LDLR in the liver for 1-14 days, 2-14 days, 3-14 days, 7-14 days, 1-10 days, 2-10 days, 3-10 days, 7-10 days, 1-7 days, 2-7 days, 3-7 days, 1-4 days, 1-3 days, or about 24 hours to about 48 hours.

In some embodiments, the method includes administering an agent (e.g., any of the siRNAs or ASOs disclosed herein) in a delivery molecule. In some embodiments, the delivery molecule is lipid nanoparticle (LNP), a lipoplex, an adenoviral vector, or an AAV, optionally wherein the AAV is an AAV1, AAV2, AAV3, AAV4, AAV5, AAV6, AAV7, AAV8, AAVrh10, AAVrh74, AAV9, AAV9P, or Myo-AAV. In some embodiments, the method enhances their delivery and/or uptake by the liver. In some embodiments, the method includes administering siRNA conjugated to a lipid, such as cholesterol-conjugated siRNAs. In some embodiments, the method includes administering siRNA conjugated to at least one galactose or galactose derivative, including but not limited to lactose, galactose, N-acetylgalactosamine (GalNAc), GalNAc-6 (doi: 10.1038/s41467-023-37465-1), galactosamine, N-formylgalactosamine, N-acetylgalactosamine, N-propionylgalactosamine, Nn-butanoylgalactosamine, and N-isobutanoyl-galactosamine (Iobst, S T and Drickamer, K. JBC 1996, 271, 6686). In some embodiments, the method includes administering GalNAc-conjugated siRNAs. In some embodiments, the method includes administering GalNAc-6-conjugated siRNAs.

In some embodiments, the method includes administering a composition comprising siRNA delivered by non-viral tissue-specific delivery vehicles including but not limited to nanoparticles, liposomes, ribonucleoproteins, positively charged peptides, small molecule RNA-conjugates, aptamer-RNA chimeras, and RNA-fusion protein complexes.

Exemplary modes of administration of the composition for blocking LDL binding to LDL receptors in the liver, e.g., a conjugated siRNA or siRNA-LNP delivery system, include oral administration, parenteral administration, administration by injection (e.g., intravenous, subcutaneous, intramuscular, intrathecal (IT), intracerebroventricular (ICV), etc.), and any other suitable mode of administration. In some embodiments, administration of the agent for blocking LDL binding to LDL receptors in the liver (e.g., siRNA) and compositions thereof include intraocular administration, such as intravitreal, intraretinal, subretinal, subtenon, peri- and retro-orbital, trans-corneal and trans-scleral administration. In some embodiments, siRNA may be administered to a patient by intravenous injection, subcutaneous injection, oral delivery, liposome delivery or intranasal delivery. The siRNA may then accumulate in a target body system, organ, tissue or cell type of the patient.

In some embodiments, other drugs that facilitate increased uptake of an agent (e.g., siRNA) in the liver may also be co-administered with the agent (e.g., siRNA) conjugated to a liver-targeting moiety. In some embodiments, the method comprises co-administering a cholesterol-conjugated agent (e.g., siRNA) with a statin drug to block LDL binding to LDLR in the liver. In some embodiments, a statin drug can be co-administered with the cholesterol-agent to enhance uptake of cholesterol-conjugated agent in the liver. See US20150361432A1 for a general discussion regarding co-administering statin drugs with cholesterol-siRNA to increase the expression of LDL receptors on the surface of liver hepatocytes. As a consequence of the increase in LDL receptor expression, the level of cholesterol is lowered in plasma. Without wishing to be bound by theory, by administering a statin drug, the level of competing cholesterol in plasma is reduced and the level of LDL receptors for binding cholesterol-agent in the liver are increased, allowing for more efficient uptake of cholesterol labeled agent by hepatocytes. The statin can be administered before, with or after the administration of the cholesterol-agent.

In some embodiments, the method includes administering an LNP or lipoplex comprising a payload to the subject, including any of the payloads described herein. In some embodiments, the payload targets a non-liver tissue. In some embodiments, the method comprises administering to a subject the composition comprising an agent that blocks LDL binding to an LDL receptor (LDLR) and/or prevents LNP uptake into liver cells or tissue as part of the pre-conditioning treatment prior to receiving or administering the payload. In some embodiments, the payload is a component of a CRISPR/Cas system or a nucleic acid encoding one or more components thereof, a biologic, or a small molecule, optionally wherein the component of a CRISPR/Cas system comprises one or more guide RNAs, one or more scaffolds, and/or one or more endonucleases. In some embodiments, the payload is for gene therapy directed to the non-liver target tissue. In some embodiments, the composition for blocking LDL binding to LDL receptors in the liver and/or for preventing LNP uptake into liver cells or tissue is administered to a subject at least once before the administration of the payload directed to the non-liver target tissue. In some embodiments, the composition for blocking LDL binding to LDL receptors in the liver and/or for preventing LNP uptake into liver cells or tissue is administered to a subject at least once before the payload-based gene therapy as part of a pre-conditioning regimen that may include administering other agents.

In some embodiments, the payload that is administered comprises a component of a CRISPR/Cas system or a nucleic acid encoding one or more components thereof, a biologic, or a small molecule, optionally wherein the component of a CRISPR/Cas system comprises a nucleic acid encoding a guide RNA and/or an endonuclease for gene editing. The non-liver payload-based gene therapy includes treating or preventing a disease or disorder, such as a genetic disease or disorder, in a subject in need thereof that is not a disease or disorder of the liver. In some embodiments, the payload is intended for the brain; central nervous system; spinal cord; eye; retina; bone; cardiac muscle, skeletal muscle, and/or smooth muscle; lung; pancreas; heart; and/or kidney. In some embodiments, the payload is intended for cardiac muscle, skeletal muscle, and/or smooth muscle.

In particular embodiments, the payload that is administered is in a lipid nanoparticle (LNP). In some embodiments, the LNP targets the brain; spinal cord; eye; retina; bone; cardiac muscle, skeletal muscle, and/or smooth muscle; lung; pancreas; heart; and/or kidney. In some embodiments, the LNP targets cardiac muscle, skeletal muscle, and/or smooth muscle.

In some embodiments, the method results in an increased percentage of payload delivered to the non-liver target. In some embodiments, the method results in at least a 10%, 30%, 50%, 75%, 100%, 125%, 150%, 200%, 250%, 300%, 350%, 400%, 450%, 500%, 600%, 700%, 800%, 900%, or 1000% increase of payload in a non-liver target, including the brain, spinal cord, eye, retina, bone, cardiac muscle, skeletal muscle, and/or smooth muscle, lung, pancreas, heart, and/or kidney of the subject receiving the agent that blocks LDL binding to an LDL receptor (LDLR) in the liver and/or prevents LNP uptake into liver cells or tissue and subsequently a payload, as compared to the payload in the corresponding tissue of a control subject that received the payload but did not receive the agent that blocks LDL binding to an LDL receptor (LDLR) in the liver and/or prevents LNP uptake into liver cells or tissue. In some embodiments, the method results in a 10-50%, 50-100%, 100-250%, 250-500%, 500-750%, 750-1000%, or 1000-2000% increase of payload in a non-liver target, including the brain, central nervous system, spinal cord, eye, retina, bone, cardiac muscle, skeletal muscle, smooth muscle, lung, pancreas, heart, and/or kidney of the subject receiving the agent that blocks LDL binding to an LDL receptor (LDLR) in the liver and/or prevents LNP uptake into liver cells or tissue and subsequently a payload as compared to the payload in the corresponding tissue of a control subject that received the payload but did not receive the agent that blocks LDL binding to an LDLR receptor (LDLR) in the liver and/or prevents LNP uptake into liver cells or tissue.

In some embodiments, the method results in at least a 10%, 30%, 50%, 75%, 100%, 125%, 150%, 200%, 250%, 300%, 350%, 400%, 450%, 500%, 600%, 700%, 800%, 900%, or 1000% increase of payload in skeletal muscle of the subject receiving the agent that blocks LDL binding to an LDL receptor (LDLR) in the liver and/or prevents LNP uptake into liver cells or tissue and subsequently a payload, as compared to the payload in the muscle of a control subject that received the payload but did not receive the agent. In some embodiments, the method results in a 10-50%, 50-100%, 100-250%, 250-500%, 500-750%, 750-1000%, or 1000-2000% increase of payload in skeletal muscle of the subject receiving the agent that blocks LDL binding to an LDL receptor (LDLR) in the liver and subsequently a payload, as compared to the payload in the muscle of a control subject that received the payload but did not receive the agent.

In some embodiments, the non-liver payload-based therapy is used to treat a genetic disease or disorder, where the disorder is a muscle disease or disorder. The muscle disease or disorder may be selected from, for example, Duchenne Muscular Dystrophy (DMD), Becker muscular dystrophy (BMD), Emery-Dreifuss dystrophy, myotonic dystrophy, limb-girdle muscular dystrophy, oculopharyngeal muscular dystrophy, congenital dystrophy, familial periodic paralysis. In some embodiments, the muscle disease or disorder may be mitochondrial oxidative phosphorylation disorder, or a glycogen storage disease (e.g., von Gierke's disease, Pompe's disease, Forbes-Cori disease, Andersen's disease, McArdle's disease, Hers' disease, Tarui's disease, or Fanconi-Bickel syndrome.) In particular embodiments, the non-liver payload-based gene therapy is used to treat DMD. In some embodiments, the non-liver payload-based therapy is used to treat myotonic dystrophy.

In some embodiments, the method includes administering a single payload or multiple payloads that have a non-liver target. In some embodiments, the payload-based therapy involves administering CRISPR-Cas components, any of which are known in the art. In some embodiments, the method includes administering one or more payloads comprising a nucleic acid encoding a Cas9 protein. Such embodiments include for example, one or more nucleic acids encoding Staphylococcus aureus (SaCas9) and/or Staphylococcus lugdunensis (SluCas9) and further comprising a nucleic acid encoding one or more guide RNAs. In such embodiments, the nucleic acid encoding the Cas9 protein is under the control of a CK8e promoter. In some embodiments, the nucleic acid encoding the guide RNA sequence is under the control of a hU6c promoter. In some embodiments, in addition to guide RNA and Cas9 sequences, the payloads further comprise nucleic acids that do not encode guide RNAs. In some embodiments, the payload vector comprises one or more nucleotide sequence(s) encoding a crRNA, a trRNA, or a crRNA and trRNA. A discussion of different payload compositions useful in the present methods, including exemplary guide RNAs, promoters, and particular spacer sequences are disclosed in WO2022/056000 and elsewhere herein.

In particular embodiments, the non-liver target is the muscle, and the method comprises administering a payload to the subject subsequent to administering to a subject an agent that blocks LDL binding to an LDL receptor (LDLR) in the liver and/or prevents LNP uptake into liver cells or tissue. In particular embodiments, the method comprises administering a payload targeting the muscle subsequent to a pre-conditioning step comprising administering to the subject a composition comprising an agent that blocks LDL binding to an LDL receptor (LDLR) in the liver and/or prevents LNP uptake into liver cells or tissue, wherein the pre-conditioning step increases the percentage of payload delivered to the non-liver target. In particular embodiments, the non-liver payload-based gene therapy is used to treat DMD. In such embodiments, the guide RNAs comprise as non-limiting examples the guide sequences disclosed in Tables 1A, 1B, and Table 2. For example, when the payload comprises SaCas9, one or more spacer sequences is selected from any one of SEQ ID NOs: 1-35, 1000-1078, and 3000-3069; or when the payload vector comprises SluCas9, one or more spacer sequences selected from any one of SEQ ID NOs: 100-225, 2000-2116, and 4000-4251.

In some embodiments, the methods include administering a payload that further comprises molecules for enhancing tropism for the target host cells or tissue. Uptake of the payload by vascular endothelial and other target cell types can further be enhanced by using payloads that further comprise one or more molecules for enhancing tropism of the vector for particular target host cells. In some embodiments, the one or more molecules for enhancing tropism are proteins. In some embodiments, the one or more molecules for enhancing tropism of the viral vector are peptides. In some embodiments, the one or more peptides target the viral vector to proteins upregulated in cells associated with the particular genetic disease or disorder to be treated. Such peptides and proteins are known in the art for enhancing tropism toward target host cells and may be incorporated into the payload through any of various methodologies known in the art.

Exemplary modes of administration of the non-liver payload-based gene therapy include oral, rectal, transmucosal, topical, transdermal, inhalation, parenteral (e.g., intravenous, subcutaneous, intradermal, intramuscular, and intra-articular, as well as direct tissue or organ injection, alternatively, intrathecal, direct intramuscular, intraventricular, intravenous, intraperitoneal, intranasal, or intraocular injections. Injectables can be prepared in conventional forms, either as liquid solutions or suspensions, solid forms suitable for solution or suspension in liquid prior to injection, or as emulsions. Alternatively, a virus may be administered locally, for example in a depot or sustained-release formulation.

In some embodiments, the subject is a human subject. In some embodiments, the subject is being treated for a genetic disease or a disorder that is not a disease or disorder of the liver. In some embodiments, the subject is being treated for a muscle disease or disorder. In some embodiments, the subject has been or is being treated with a non-liver payload-based gene therapy.

### EXAMPLES

The following examples are provided to illustrate certain disclosed embodiments and are not to be construed as limiting the scope of this disclosure in any way.

### 1. Example 1: Study of the Effect of LDLR mRNA Knockdown in Liver Cells

To evaluate the knockdown effects of anti-LDLR siRNA in liver cells, liver cell models were transfected with siRNAs targeting LDLR (anti-LDLR siRNA). Cells were lysed for mRNA and protein extraction followed by qRT-PCR (mRNA) analysis to assess the degree of LDLR mRNA knockdown.

### a) Dose Response (CRO)

Hepa1-6 cells were seeded at a density of 20 k cells per well in 96-well tissue culture plates. Cells were immediately transfected with siRNAs targeting mmLDLR (Mus musculus low density lipoprotein receptor, Gene ID: 16835) at 10 different doses using Lipofectamine 2000 (Invitrogen 11668027). A Quantigene 2.0 branched DNA (bDNA) probe set was designed for the target mRNAs. Relative mmLdlr/mmGAPDH ratios were normalized to the respective mean ratio in mock treated cells and cells were transfected with a control siRNA targeting.

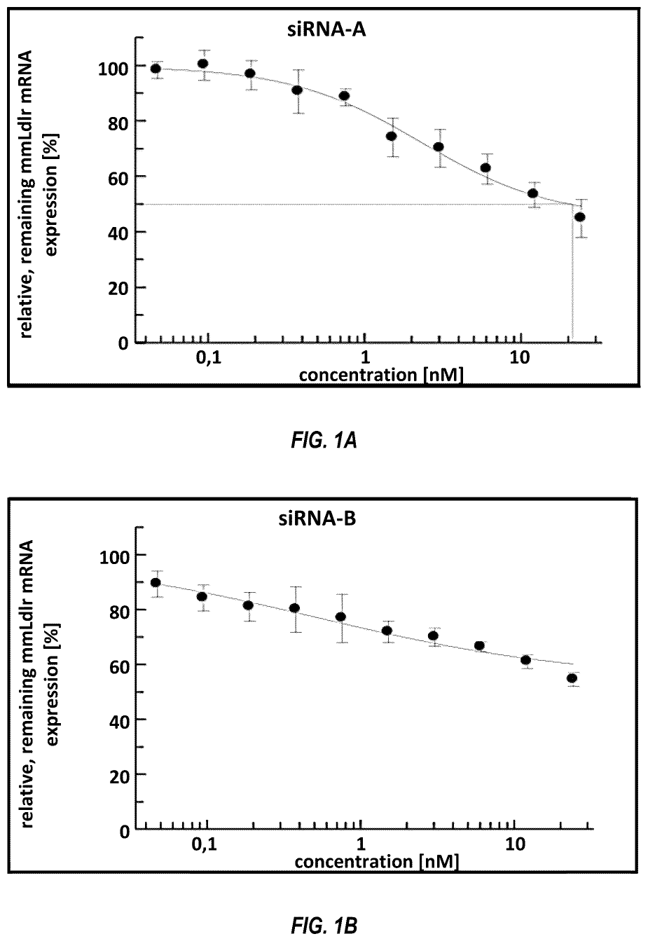

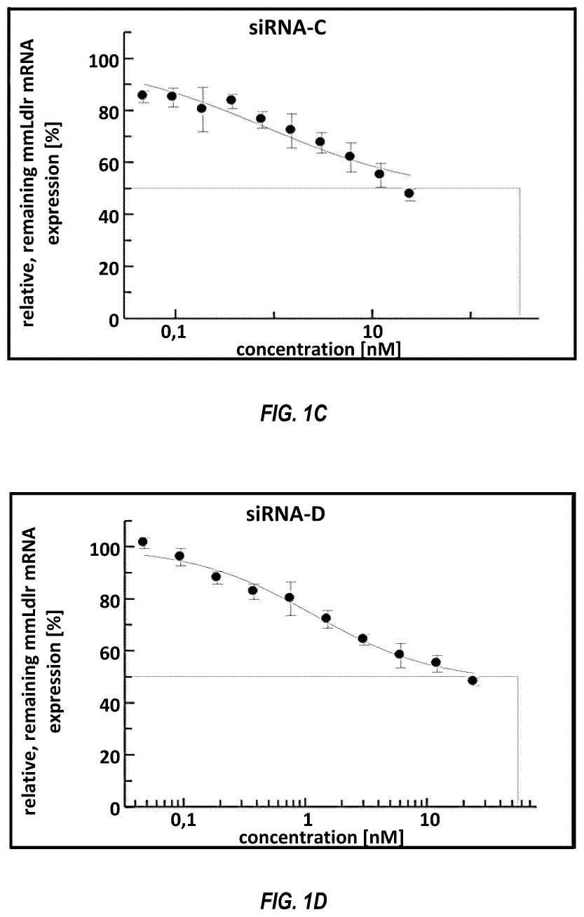

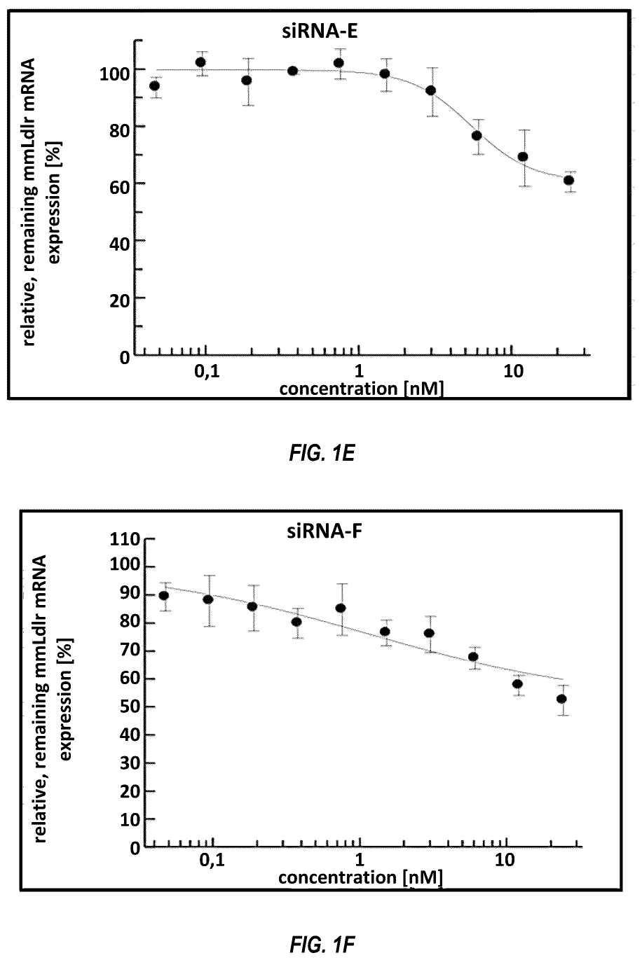

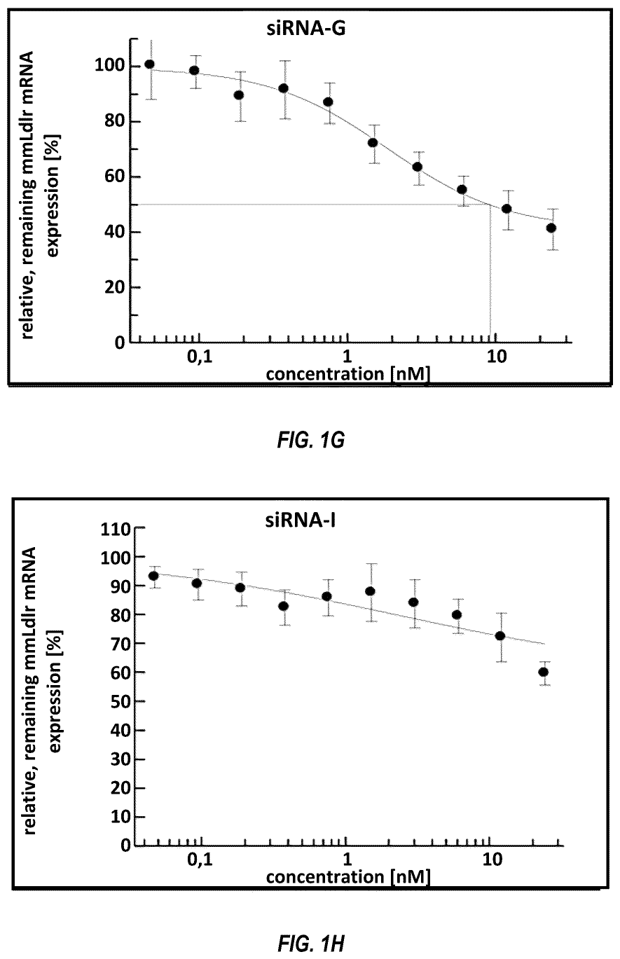

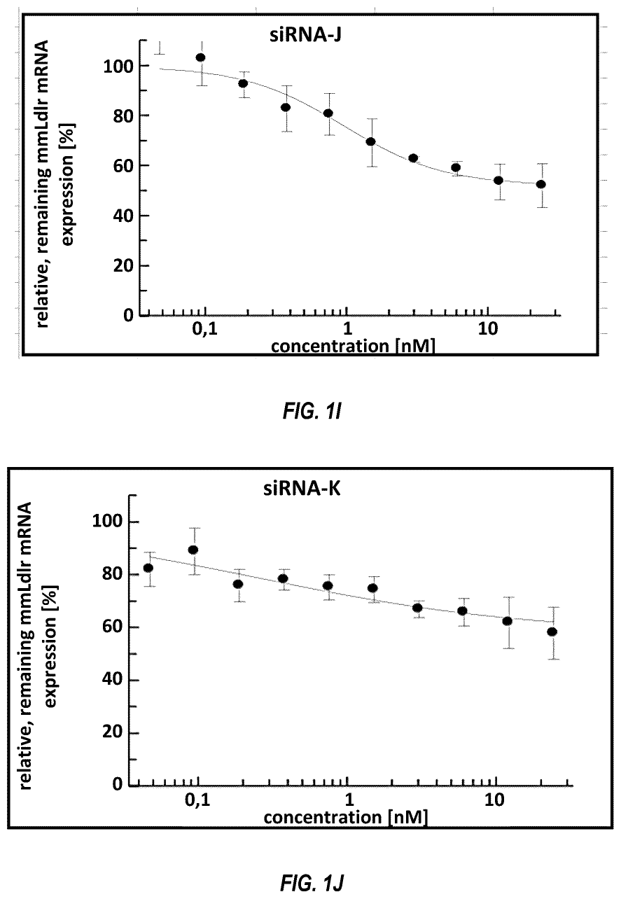

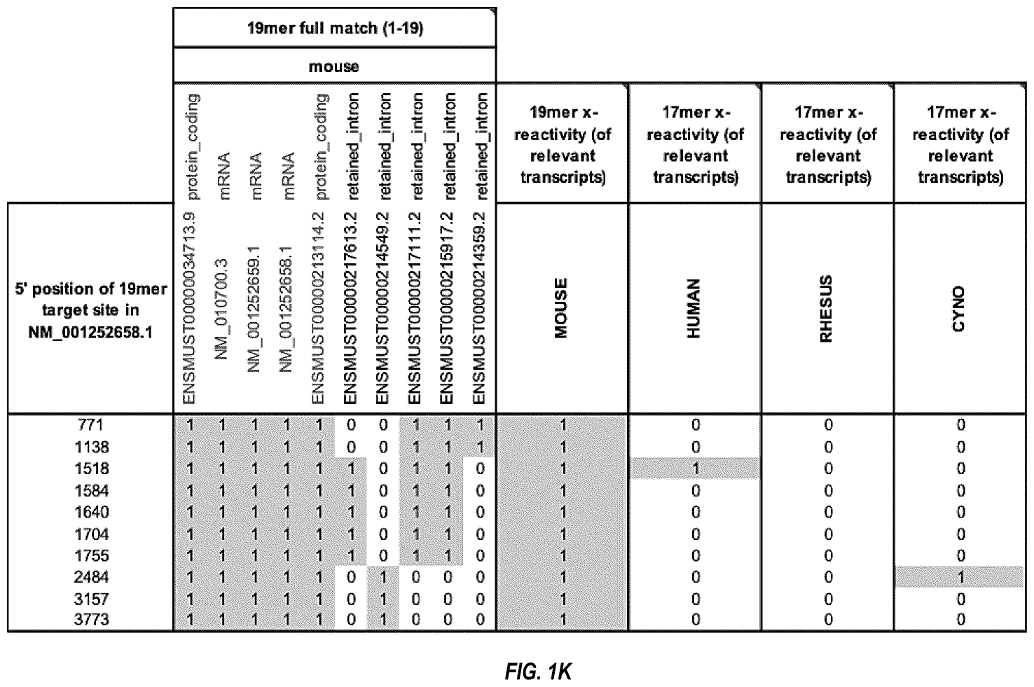

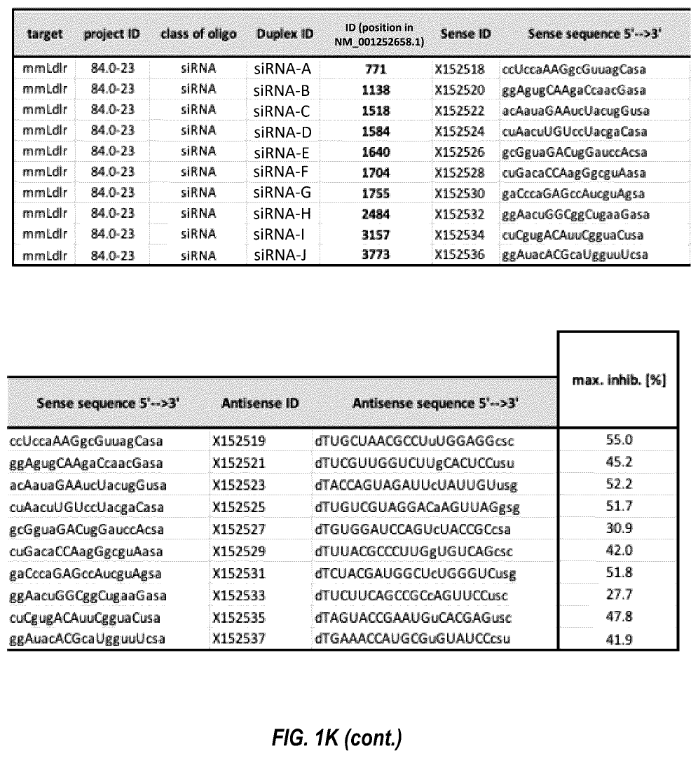

FIGS. 1A-1J show the effects of siRNA concentration on relative mRNA expression and show 10-point dose response curves for each of the 10 siRNA sequences evaluated, set forth in Table 3A (wherein each duplex siRNA shown comprises a pair of amino acid sequences of SEQ ID NOs: 4300 and 9965 [FIG. 1A], 4301 and 9966 [FIG. 1B], 4302 and 9967 [FIG. 1C], 4303 and 9968 [FIG. 1D], 4304 and 9969 [FIG. 1E], 4305 and 9970 [FIG. 1F], 4306 and 9971 [FIG. 1G], 4307 and 9972 [FIG. 1H], 4308 and 9973 [FIG. 1I], or 4309 and 9974 [FIG. 1J]). The top performing mmLDLR-targeting siRNAs were used in subsequent studies.

b) Candidate siRNA Screening LDLR in Hepa1-6 Cells

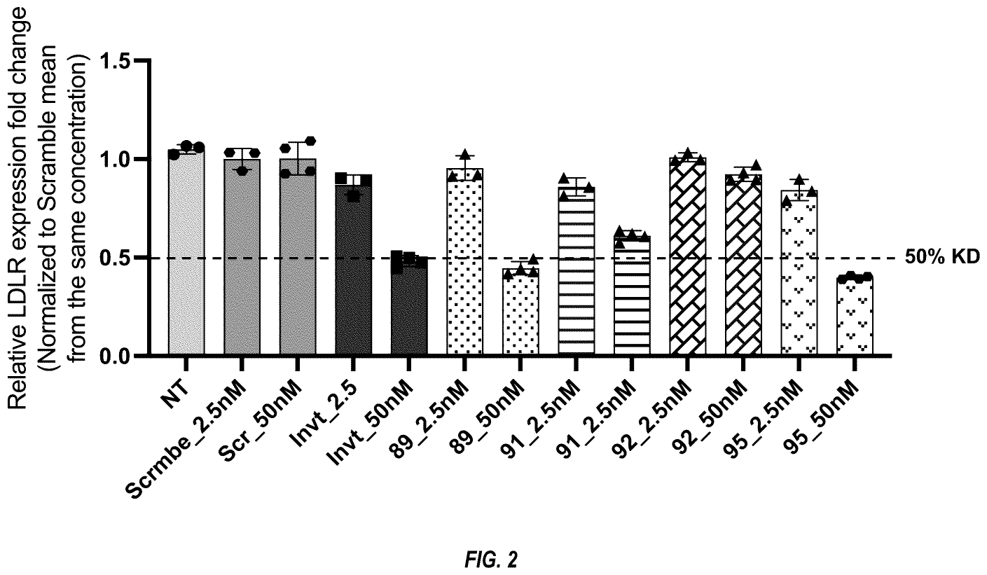

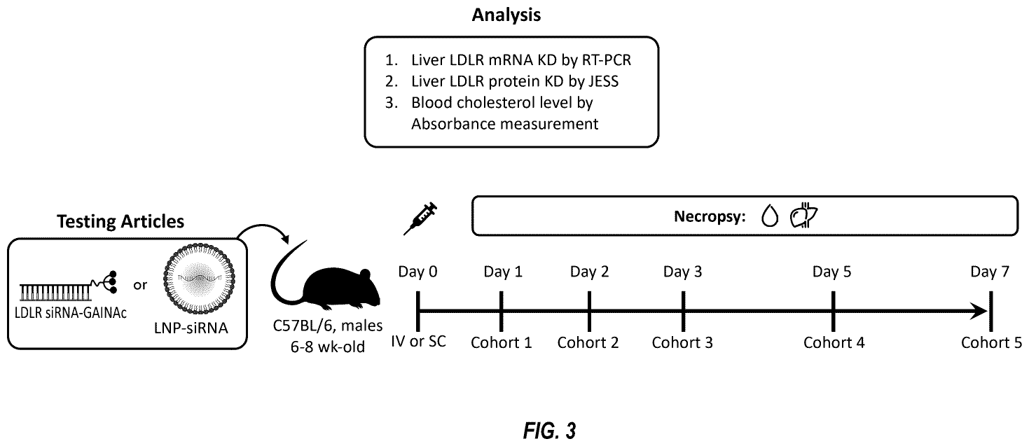

Hepa1-6 cells were seeded at 15 k per well in a 96-well tissue culture plate and grown for 48 h in growth media DMEM supplemented with 10% FBS and PenStrep. The next day, cells were treated with 2.5 nM (left column of modality in FIG. 2) or 50 nM (right column of modality in FIG. 2) of siRNA mixed with Opti-MEM (GIBCO, 31985062) and combined with mixture of Lipofectamine RNAiMAx Transfection reagent (Invitrogen, 13778150) and incubated for 15 min at room temperature as per the manufacturer procedure. Untreated cells were left until the media was changed.

After 48 hours post-dosing, cells were frozen at −80 degrees C., and then lysed using lysis buffer from Cells-to-CT 1-step TaqMan Kit (Invitrogen A25603). qPCR was run on Quantstudio 6 flex using mastermix from Cells-to-CT 1-step TaqMan Kit (Invitrogen A25603). 20 ul reactions were conducted in technical triplicate or quadruplicates for each RNA sample according to user manual. 2 ul of lysate was added to each reaction and Taqman probes (Mus muscus): LDLR: FAM Mm01151337_m1 and Act-B: VIC Mm02619580_g1 were used. Delta Delta Ct analysis was conducted to find fold change in relation to untreated control samples.

FIG. 2 shows the amount of relative LDLR expression fold change normalized to scramble mean from the same concentration.

### 2. Example 2: In Vivo Study to Determine Kinetics of Liver LDLR Knockdown by GalNAc-Conjugated siRNA or siRNA-LNP

To evaluate the effects of anti-LDLR siRNA administration on liver LDLR knockdown in a mouse model, wild-type mice was pre-conditioned with either a siRNA targeting LDLR (anti-LDLR) with GalNAc (siRNA-GalNAc) or a liver-targeted siRNA-LNP.

A single dose of SiRNA-LNP or siRNA-GalNac targeting LDLR was administrated via subcutaneous (siRNA-GalNAc) or intravenous (siRNA-LNP), routs of administration at the dose of 20 mg/kg for siRNA-GalNAc and 3 mg/kg for siRNA-LNP, and the mice were euthanized at 1-, 2-, 3-, 5-, or 7-day post injection for sample analysis.

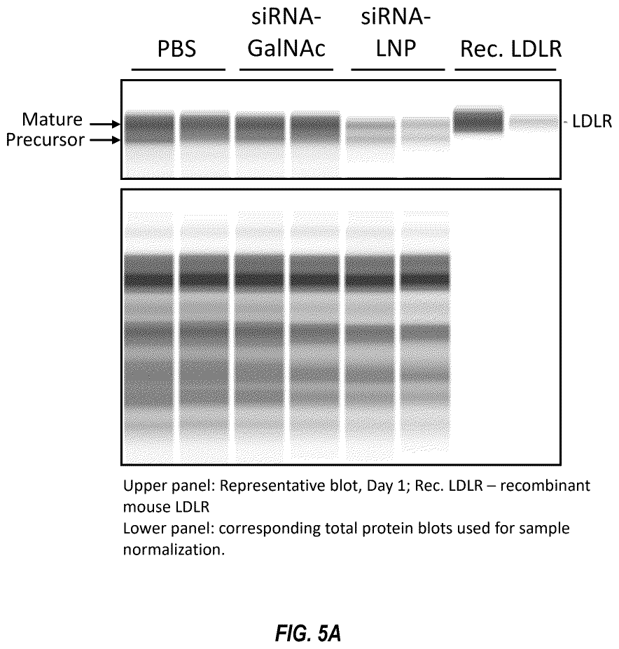

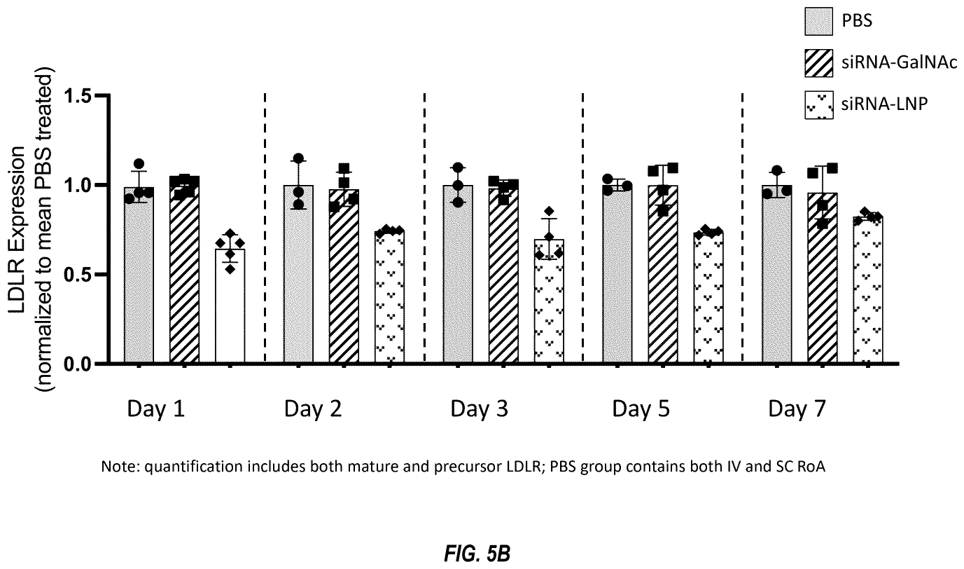

As described below, the level of LDL receptor (LDLR) protein in liver was assessed via the capillary gel electrophoresis system ProteinSimple “Jess” and relative RT-qPCR measurement of LDLR mRNA. LDL-cholesterol level was measured using Fujifilm Wako colorimetric kit. A representative image of Jess blot for selected samples from Day 1 and LDLR recombinant protein is shown in FIG. 5A.

### a) LNP Formulation

The LNP lipid composition by mole fraction was: 50% LP-01 (BP-26809, BroadPharm) as the ionizable lipid, 9% DSPC (850365, Avanti Polar Lipids), 39% Cholesterol (C3045, Milipore Sigma), and 2% DMG-PEG2000 (880151, Avanti Polar Lipids). The duplex siRNA with a sense sequence comprising SEQ ID NO: 4306 and an antisense sequence comprising SEQ ID NO: 9971 was loaded as cargo.

### b) Formulation

The siRNA solution in 50 mM Citrate buffer at pH 4.5 buffer (aqueous phase) and lipid mixture in ethanol (lipid phase) was loaded onto microfluidic mixing device (Ignite, NanoAssembler, Precision Nanosystems) and run at 3:1 (RNA to lipid) flow rate with total flow rate of 12 mL/min. The LNP were collected and left at 4 degrees C. for 30 min for maturation, dialyzed in 50 mM Tris Buffer (pH 7.5) and concentrated and frozen in 100 mM sucrose at −80 C.

c) LDLR mRNA Knockdown

Mouse liver tissues, prepared as described above, were frozen at −80 degrees C. at corresponding time points. RNA was extracted from tissues using homogenization and lysis buffer from Cells-to-CT 1-step TaqMan Kit (Invitrogen A25603). qPCR was run on Quantstudio 6 flex using mastermix from Cells-to-CT 1-step TaqMan Kit (Invitrogen A25603). 20 ul reactions were conducted in technical triplicates for each RNA sample according to the user manual. 2 ul of lysate was added to each reaction and Taqman probes targeting: LDL-r Mm01151337_m1 LDLR, and B-actin (house keeping gene): Mm02619580_g1 were used. Delta Delta Ct analysis was conducted to find fold change in relation to untreated control sample.

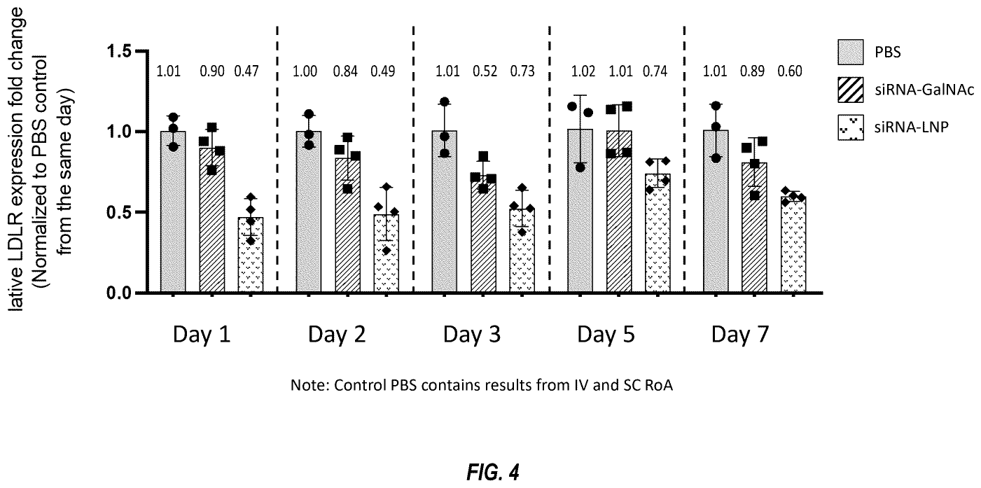

Results are shown in FIG. 4.

### d) LDLR Protein Knockdown

The LDLR levels in protein lysate derived from mouse liver tissue, prepared as described above, were quantitated on a capillary western system, which is an automated plate-based, transfer-free system.

Briefly, protein was extracted from frozen tissue (10-20 mg) using a BeadRuptor bead mill homogenizer submerged in 10% SDS buffer (1 mM EDTA pH8, 100 mM NaCl, 62.5 mM Tris pH 6.5, 10% SDS, 10% Glycerol, and water) supplemented with HALT Protease Inhibitor, 3 homogenization cycles run for 20 s at 6 m/s. The extracted protein lysate concentration was determined using a BCA Protein Concentration kit (Thermofisher, 23225) according to the manufacturers' recommendation. Following protein concentration determination, samples were prepared for Jess Abby & Wes Separation module (“Jess”), according to manufacturer protocol from SM-W001. The lysates were diluted to 0.5 mg/mL with 0.1× sample buffer and mixed with a fluorescent master mix to a final concentration of 0.4 mg/mL. A loading volume of 3 μL (1.2 pg total protein) per sample was then added onto the plate. The biotinylated molecular weight ladder (12-230 kDa), antibodies, and total protein detection reagents were prepared and pipetted onto the plate per protocol before placing the plate into Jess. LDLR was detected with commercial primary antibody (Abcam ab30532) at 1:25 dilution and used with matching ProteinSimple secondary antibody (Anti-rabbit HRP, ProteinSimple, 042-206) at ready to use dilution. A recombinant Mouse LDLR Protein was used as a surrogate for an assay control (R&D Systems, 2255-LD) with two QC levels at 750 ng/mL and 375 ng/mL.

Voltage was applied to induce protein migration and separation based on molecular weight, followed by detection with primary/secondary antibodies and total protein via electrochemiluminescence (ECL). The Area Under the Curve (AUC) of the LDLR signal for all samples was normalized to the AUC of a total protein AUC. LDLR signal from test article-dosed groups was then normalized against the mean LDLR signal of the vehicle-treated group.

Results are shown in FIGS. 5A-B. Compared to PBS, siRNA-LNP results in sustainable knockdown of LDLR protein through day 7.

### e) Blood Cholesterol Biomarker

Briefly, 210 μL of Reagent 1 (993-00404, FUJIFILM Wako Chemicals) and 2.4 L of sample (fresh blood collected from tail vein), calibrator (990-28011, FUJIFILM Wako Chemicals), or blank (0.9% saline) into each well (as needed) of a 96-well microplate, mixed and incubated at 37 degrees C. for 5 min following Absorbance measurement at 600 nm wavelength (microplate reader). Followed by addition of 70 L of Reagent 2 (993-00504, FUJIFILM Wako Chemicals), 5 min incubation in 37 degree c. and second Absorbance measurement at 600 nm. The LDL-c concentration in the sample was calculated by comparison of the result from Calibrator and normalized to mean PBS value of untreated animals from given timepoint.

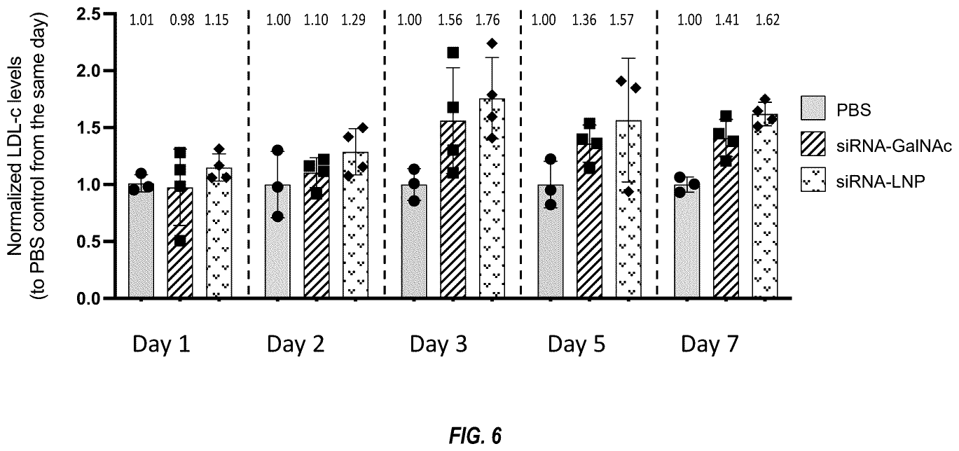

Results are shown in FIG. 6.

### 3. Example 3: In Vivo Dose Range Study

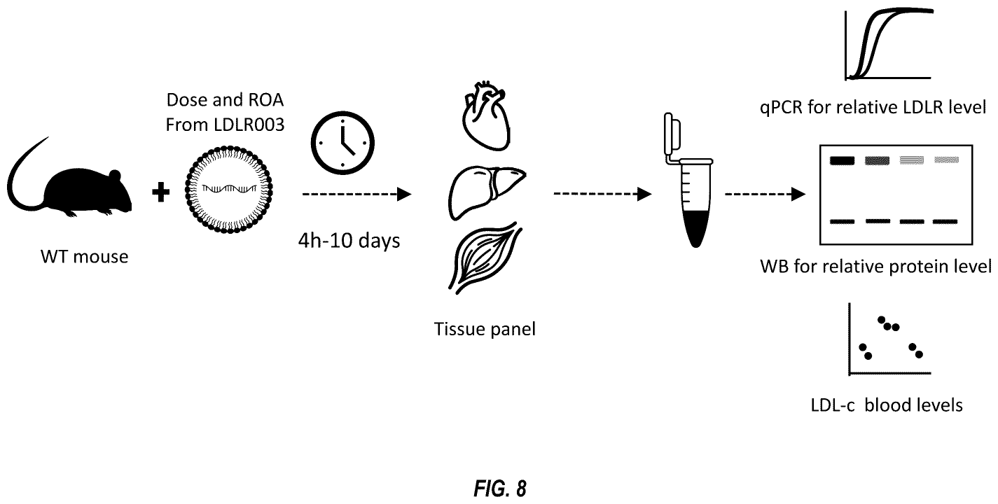

As shown in FIG. 8, the efficacy of LDLR KD will be evaluated in 6-8 weeks old Wildtype (WT) mice for dose selection. A single dose liver tropic LDLR targeting siRNA-LNP will be administrated at the dose range between 0.1 to 25 mg/kg, and the mice will be euthanized 4-48 h post injection for sample analysis.

The level of LDL receptor (LDLR) protein in liver will be assessed via the capillary gel electrophoresis system ProteinSimple “Jess”; the mRNA expression level of LDLR in liver will be evaluated using RT-qPCR. LDL-cholesterol level in blood will be measured using Colorimetric Absorbance assay using FUJIFILM Wako Chemicals USA, Corp.

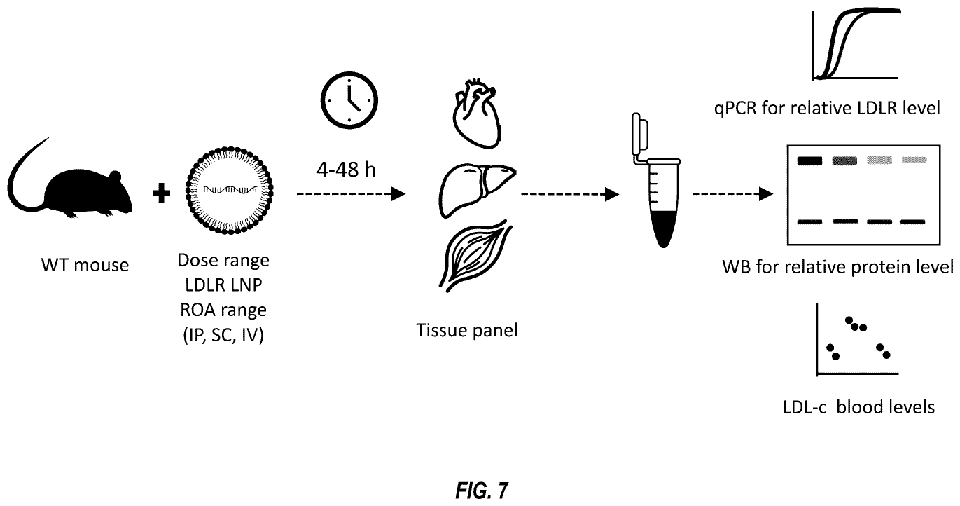

A graphical representation of the study is shown in FIG. 7.

### 4. Example 4: In Vivo Kinetic Study

An in vivo study will be conducted to examine LDLR protein knockdown kinetics. The kinetic of LDLR KD will be evaluated in 6-8 weeks old wild-type (WT) mice. A single dose of LNP with LDLR targeting cargo will be administrated at the dose selected from the dose range in vivo study, described above in Example 3, and the mice will be euthanized at 4 h to 10-day post injection for sample analysis.

The level of LDL receptor (LDLR) protein in liver will be assessed via the capillary gel electrophoresis system ProteinSimple “Jess”; the mRNA expression level of LDLR in liver will be evaluated using RT-qPCR. LDL-cholesterol level in blood will be measured using Colorimetric Absorbance assay using FUJIFILM Wako Chemicals USA, Corp.

A graphical representation of the study is shown in FIG. 8.

### 5. Example 5: Proof-of-Concept Study

Briefly, wild-type (WT) mice will be dosed with a liver tropic LNP with cargo targeting LDLR, dose and ROA determined from the dose range in vivo study, described above in Example 3, followed up by administration of therapeutic LNP at a time point based on the most efficient LDLR knock down time determined from the in vivo kinetic study, described above in Example 4.

The animals will be euthanized for tissue panel collection and analysis of LDLR KD and therapeutic LNP biodistribution between 4 hours and 10 days post-dosing with therapeutic LNP. Control animals will be dosed with PBS in place of liver tropic LNP with cargo targeting LDLR and/or therapeutic LNP and sacrificed at a given time point.

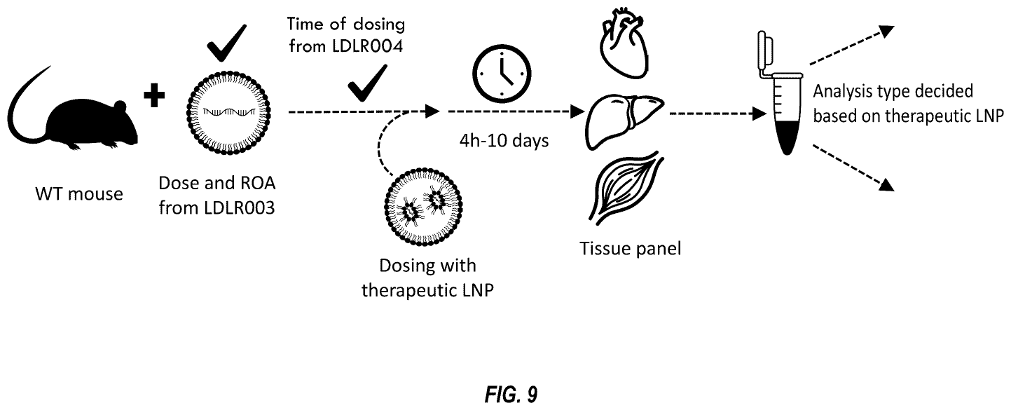

A graphical representation of the study is shown in FIG. 9.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed item.

In Table 3A, the following monikers are used to describe the components of specific siRNA sequences. These bases and modifications are taken to be non-limiting. Uppercase characters (e.g., A, U, C, G) represent RNA bases. Lowercase characters (e.g., a, u, c, g) represent 2′ O-methyl RNA bases. Characters preceded by “d” (“d_”) represent DNA bases. Characters preceded by “s” (“s_”) represent phosphorothioate linkages.

TABLE 3A
Exemplary siRNA Sequences

| SEQ | | SEQ | |
|---|---|---|---|
| ID | Sense Sequence | ID | Antisense Sequence |
| NO. | (5′ to 3′) | NO. | (5′ to 3′) |
| 4300 | ccUccaAAGgcGuuagCasa | 9965 | dTUGCUAACGCCUuUGGAGGcsc |
| 4301 | ggAgugCAAgaCcaacGasa | 9966 | dTUCGUUGGUCUUgCACUCCusu |
| 4302 | acAauaGAAucUacugGusa | 9967 | dTACCAGUAGAUUcUAUUGUusg |
| 4303 | cuAacuUGUccUacgaCasa | 9968 | dTUGUCGUAGGACaAGUUAGgsg |
| 4304 | gcGguaGACugGauccAcsa | 9969 | dTGUGGAUCCAGUCUACCGCcsa |
| 4305 | cuGacaCCAagGgcguAasa | 9970 | dTUUACGCCCUUGgUGUCAGcsc |
| 4306 | gaCccaGAGccAucguAgsa | 9971 | dTCUACGAUGGCUCUGGGUCusg |
| 4307 | ggAacuGGCggCugaaGasa | 9972 | dTUCUUCAGCCGCcAGUUCCusc |
| 4308 | cuCgugACAuuCgguaCusa | 9973 | dTAGUACCGAAUGuCACGAGusc |
| 4309 | ggAuacACGcaUgguuUcsa | 9974 | dTGAAACCAUGCGuGUAUCCcsu |

TABLE 3B
Exemplary siRNA Sequences

| SEQ ID | | SEQ ID | |
|---|---|---|---|
| NO. | antisense_5to3_rna | NO. | antisense_5to3_dna |
| 4310 | AAAAAAUCCCCACUAUCUGUA | 9975 | AAAAAATCCCCACTATCTGTA |
| 4311 | AAAAACCUUCUAUACAGUGGU | 9976 | AAAAACCTTCTATACAGTGGT |
| 4312 | AAAAAGCGUUGCACACAGUCC | 9977 | AAAAAGCGTTGCACACAGTCC |
| 4313 | AAAAAGUGACACCCAUCUCCC | 9978 | AAAAAGTGACACCCATCTCCC |
| 4314 | AAAAAUAAAUAGAUUUUAUAU | 9979 | AAAAATAAATAGATTTTATAT |
| 4315 | AAAAAUCCCCACUAUCUGUAC | 9980 | AAAAATCCCCACTATCTGTAC |
| 4316 | AAAACAAAGCUCUGGCAGGCA | 9981 | AAAACAAAGCTCTGGCAGGCA |
| 4317 | AAAACAAGGCCGGCGAGGUCU | 9982 | AAAACAAGGCCGGCGAGGTCT |
| 4318 | AAAACAGUGCAAAAUAAUAAC | 9983 | AAAACAGTGCAAAATAATAAC |
| 4319 | AAAACCUUCUAUACAGUGGUU | 9984 | AAAACCTTCTATACAGTGGTT |
| 4320 | AAAACUUCCUGGAGAGAAAUG | 9985 | AAAACTTCCTGGAGAGAAATG |
| 4321 | AAAAGCGUUGCACACAGUCCA | 9986 | AAAAGCGTTGCACACAGTCCA |
| 4322 | AAAAGUGACACCCAUCUCCCA | 9987 | AAAAGTGACACCCATCTCCCA |
| 4323 | AAAAUAAAUAGAUUUUAUAUA | 9988 | AAAATAAATAGATTTTATATA |
| 4324 | AAAAUAAUAACACAAAUGCCA | 9989 | AAAATAATAACACAAATGCCA |
| 4325 | AAAAUACUUUGUCCUCAAAGA | 9990 | AAAATACTTTGTCCTCAAAGA |
| 4326 | AAAAUCCCAACCCAAGCCAUU | 9991 | AAAATCCCAACCCAAGCCATT |
| 4327 | AAAAUCCCCACUAUCUGUACA | 9992 | AAAATCCCCACTATCTGTACA |
| 4328 | AAAAUGGCUUCGUUGAUGAUA | 9993 | AAAATGGCTTCGTTGATGATA |
| 4329 | AAACAAAGCUCUGGCAGGCAA | 9994 | AAACAAAGCTCTGGCAGGCAA |
| 4330 | AAACAAGGCCGGCGAGGUCUC | 9995 | AAACAAGGCCGGCGAGGTCTC |
| 4331 | AAACACAUACCCAUCAACGAC | 9996 | AAACACATACCCATCAACGAC |
| 4332 | AAACACGUUGACUCCUGAGUU | 9997 | AAACACGTTGACTCCTGAGTT |
| 4333 | AAACAGUGCAAAAUAAUAACA | 9998 | AAACAGTGCAAAATAATAACA |
| 4334 | AAACAUAACAAAAAAUCCCCA | 9999 | AAACATAACAAAAAATCCCCA |
| 4335 | AAACCAAAAUCCCAACCCAAG | 10000 | AAACCAAAATCCCAACCCAAG |
| 4336 | AAACCAGAAAGGCUUAUCACG | 10001 | AAACCAGAAAGGCTTATCACG |
| 4337 | AAACCAUCCUCCCACCUCAGC | 10002 | AAACCATCCTCCCACCTCAGC |
| 4338 | AAACCCACCUCGAAAGACACU | 10003 | AAACCCACCTCGAAAGACACT |
| 4339 | AAACCCUGGCUUCCCGCGAUU | 10004 | AAACCCTGGCTTCCCGCGATT |
| 4340 | AAACCGGGGACUUCGCCAGGA | 10005 | AAACCGGGGACTTCGCCAGGA |
| 4341 | AAACCUUCUAUACAGUGGUUU | 10006 | AAACCTTCTATACAGTGGTTT |
| 4342 | AAACGAUCCAGACUGGAGGAU | 10007 | AAACGATCCAGACTGGAGGAT |
| 4343 | AAACUCAAAACUUCCUGGAGA | 10008 | AAACTCAAAACTTCCTGGAGA |
| 4344 | AAACUCGUCCUGGGAGCACGU | 10009 | AAACTCGTCCTGGGAGCACGT |
| 4345 | AAACUUCCUGGAGAGAAAUGG | 10010 | AAACTTCCTGGAGAGAAATGG |
| 4346 | AAACUUGGGCGAGUGGGGGUU | 10011 | AAACTTGGGCGAGTGGGGGTT |
| 4347 | AAAGAACCUGAAGUCCCGUCA | 10012 | AAAGAACCTGAAGTCCCGTCA |
| 4348 | AAAGAAGAGGCAUUUCAGAGG | 10013 | AAAGAAGAGGCATTTCAGAGG |
| 4349 | AAAGACACUGGGUGGGUCAUG | 10014 | AAAGACACTGGGTGGGTCATG |
| 4350 | AAAGACCCCUACAGCGCUGCG | 10015 | AAAGACCCCTACAGCGCTGCG |
| 4351 | AAAGACGACAAUGGCAGUUCU | 10016 | AAAGACGACAATGGCAGTTCT |
| 4352 | AAAGACGGCCAAGGAGAAGGG | 10017 | AAAGACGGCCAAGGAGAAGGG |
| 4353 | AAAGCAGUGGCAUAAUCAUAG | 10018 | AAAGCAGTGGCATAATCATAG |
| 4354 | AAAGCCACUUGCUUCUCUGGG | 10019 | AAAGCCACTTGCTTCTCTGGG |
| 4355 | AAAGCGUUGCACACAGUCCAG | 10020 | AAAGCGTTGCACACAGTCCAG |
| 4356 | AAAGCUCUGGCAGGCAAUGCU | 10021 | AAAGCTCTGGCAGGCAATGCT |
| 4357 | AAAGGAAGAAACCAAAAUCCC | 10022 | AAAGGAAGAAACCAAAATCCC |
| 4358 | AAAGGAAGACGAGGAGCACGA | 10023 | AAAGGAAGACGAGGAGCACGA |
| 4359 | AAAGGAAGACGAGGAGCACUG | 10024 | AAAGGAAGACGAGGAGCACTG |
| 4360 | AAAGGCUAACCUGGCUGUCUA | 10025 | AAAGGCTAACCTGGCTGTCTA |
| 4361 | AAAGGCUUAUCACGGUUCCCU | 10026 | AAAGGCTTATCACGGTTCCCT |
| 4362 | AAAGUGACACCCAUCUCCCAG | 10027 | AAAGTGACACCCATCTCCCAG |
| 4363 | AAAGUGCAAACAUAACAAAAA | 10028 | AAAGTGCAAACATAACAAAAA |
| 4364 | AAAGUGCAAGGAGACCACGGG | 10029 | AAAGTGCAAGGAGACCACGGG |
| 4365 | AAAGUGCUGGGAUUACAGGCG | 10030 | AAAGTGCTGGGATTACAGGCG |
| 4366 | AAAGUUGAUGCUGUUGAUGUU | 10031 | AAAGTTGATGCTGTTGATGTT |
| 4367 | AAAUAAAUAGAUUUUAUAUAU | 10032 | AAATAAATAGATTTTATATAT |
| 4368 | AAAUAAUAACACAAAUGCCAA | 10033 | AAATAATAACACAAATGCCAA |
| 4369 | AAAUAAUCUUUGUACAUAAAG | 10034 | AAATAATCTTTGTACATAAAG |
| 4370 | AAAUACAGCAACCAGGGUUUG | 10035 | AAATACAGCAACCAGGGTTTG |
| 4371 | AAAUACUUUGUCCUCAAAGAC | 10036 | AAATACTTTGTCCTCAAAGAC |
| 4372 | AAAUAGAUUUUAUAUAUAUGU | 10037 | AAATAGATTTTATATATATGT |
| 4373 | AAAUAGGGUCUUCAUCUGUUG | 10038 | AAATAGGGTCTTCATCTGTTG |
| 4374 | AAAUAUAUAAAACAAAGCUCU | 10039 | AAATATATAAAACAAAGCTCT |
| 4375 | AAAUAUUAAGGGUGACCAGUG | 10040 | AAATATTAAGGGTGACCAGTG |
| 4376 | AAAUCCCAACCCAAGCCAUUG | 10041 | AAATCCCAACCCAAGCCATTG |
| 4377 | AAAUCCCCACUAUCUGUACAG | 10042 | AAATCCCCACTATCTGTACAG |
| 4378 | AAAUGCCAAAUGUACACAGUG | 10043 | AAATGCCAAATGTACACAGTG |
| 4379 | AAAUGCCUGAGCUCAAACCAU | 10044 | AAATGCCTGAGCTCAAACCAT |
| 4380 | AAAUGGAGGUGUCAUCCUGGU | 10045 | AAATGGAGGTGTCATCCTGGT |
| 4381 | AAAUGGCUUCGUUGAUGAUAU | 10046 | AAATGGCTTCGTTGATGATAT |
| 4382 | AAAUGUACACAGUGUACAACU | 10047 | AAATGTACACAGTGTACAACT |
| 4383 | AAAUGUGGACCUCAUCCUCUG | 10048 | AAATGTGGACCTCATCCTCTG |
| 4384 | AAAUUCAUUUUUUAAGAGAUG | 10049 | AAATTCATTTTTTAAGAGATG |
| 4385 | AAAUUUAAUUGAUCACAGUAA | 10050 | AAATTTAATTGATCACAGTAA |
| 4386 | AACAAAAAAUCCCCACUAUCU | 10051 | AACAAAAAATCCCCACTATCT |
| 4387 | AACAAAGCUCUGGCAGGCAAU | 10052 | AACAAAGCTCTGGCAGGCAAT |
| 4388 | AACAAAUACAGCAACCAGGGU | 10053 | AACAAATACAGCAACCAGGGT |
| 4389 | AACAAGGCCGGCGAGGUCUCA | 10054 | AACAAGGCCGGCGAGGTCTCA |
| 4390 | AACAAGUUGACAUCGGAACCU | 10055 | AACAAGTTGACATCGGAACCT |
| 4391 | AACACAAAUGCCAAAUGUACA | 10056 | AACACAAATGCCAAATGTACA |
| 4392 | AACACACACGACAGAAAACAG | 10057 | AACACACACGACAGAAAACAG |
| 4393 | AACACAUACCCAUCAACGACA | 10058 | AACACATACCCATCAACGACA |
| 4394 | AACACGGCGAUUUCAGAAAGA | 10059 | AACACGGCGATTTCAGAAAGA |
| 4395 | AACACGUAAAGACCCCUACAG | 10060 | AACACGTAAAGACCCCTACAG |
| 4396 | AACACGUUGACUCCUGAGUUU | 10061 | AACACGTTGACTCCTGAGTTT |
| 4397 | AACAGAGACAGUGCCCAGGAC | 10062 | AACAGAGACAGTGCCCAGGAC |
| 4398 | AACAGCCGCCGUUGUUGUCCA | 10063 | AACAGCCGCCGTTGTTGTCCA |
| 4399 | AACAGGAUCCACCACGAUGGC | 10064 | AACAGGATCCACCACGATGGC |
| 4400 | AACAGGUCGGGUGGUUGUGUG | 10065 | AACAGGTCGGGTGGTTGTGTG |
| 4401 | AACAGUAACACGGCGAUUUCA | 10066 | AACAGTAACACGGCGATTTCA |
| 4402 | AACAGUGCAAAAUAAUAACAC | 10067 | AACAGTGCAAAATAATAACAC |
| 4403 | AACAGUUUCAACCAAUAUACA | 10068 | AACAGTTTCAACCAATATACA |
| 4404 | AACAUAACAAAAAAUCCCCAC | 10069 | AACATAACAAAAAATCCCCAC |
| 4405 | AACCAAAAUCCCAACCCAAGC | 10070 | AACCAAAATCCCAACCCAAGC |
| 4406 | AACCAAUAUACAAAGUGCAAA | 10071 | AACCAATATACAAAGTGCAAA |
| 4407 | AACCAGAAAGGCUUAUCACGG | 10072 | AACCAGAAAGGCTTATCACGG |
| 4408 | AACCAGGGUUUGCAAAAAUAA | 10073 | AACCAGGGTTTGCAAAAATAA |
| 4409 | AACCAUAUCCUCUGGGGACAG | 10074 | AACCATATCCTCTGGGGACAG |
| 4410 | AACCAUCCUCCCACCUCAGCC | 10075 | AACCATCCTCCCACCTCAGCC |
| 4411 | AACCCAAGCCAUUGCAUGGGC | 10076 | AACCCAAGCCATTGCATGGGC |
| 4412 | AACCCACCUCGAAAGACACUG | 10077 | AACCCACCTCGAAAGACACTG |
| 4413 | AACCCAGUAGAGGCGGCCACU | 10078 | AACCCAGTAGAGGCGGCCACT |
| 4414 | AACCCCCUACACAGGGCCCCG | 10079 | AACCCCCTACACAGGGCCCCG |
| 4415 | AACCCUGGCUUCCCGCGAUUG | 10080 | AACCCTGGCTTCCCGCGATTG |
| 4416 | AACCGAGGCCUUGAGGGUGGC | 10081 | AACCGAGGCCTTGAGGGTGGC |
| 4417 | AACCGCGGAGGAACGCCAGGC | 10082 | AACCGCGGAGGAACGCCAGGC |
| 4418 | AACCGGGGACUUCGCCAGGAG | 10083 | AACCGGGGACTTCGCCAGGAG |
| 4419 | AACCGGGUGUCUCAGGCACUU | 10084 | AACCGGGTGTCTCAGGCACTT |
| 4420 | AACCUCCACCUCCUGGGCUCA | 10085 | AACCTCCACCTCCTGGGCTCA |
| 4421 | AACCUCGUAAGGAACCGAGGC | 10086 | AACCTCGTAAGGAACCGAGGC |
| 4422 | AACCUGAAGUCCCGUCAAACG | 10087 | AACCTGAAGTCCCGTCAAACG |
| 4423 | AACCUGCCCCUCUCUGUCCCC | 10088 | AACCTGCCCCTCTCTGTCCCC |
| 4424 | AACCUGGCUGUCUAGCAAGGC | 10089 | AACCTGGCTGTCTAGCAAGGC |
| 4425 | AACCUGUGAGGCGGUUGGCAC | 10090 | AACCTGTGAGGCGGTTGGCAC |
| 4426 | AACCUUCUAUACAGUGGUUUA | 10091 | AACCTTCTATACAGTGGTTTA |
| 4427 | AACGACAAGAUUGGGGAAGUG | 10092 | AACGACAAGATTGGGGAAGTG |
| 4428 | AACGAUCCAGACUGGAGGAUU | 10093 | AACGATCCAGACTGGAGGATT |
| 4429 | AACGCAGCCAACUUCAUCGCU | 10094 | AACGCAGCCAACTTCATCGCT |
| 4430 | AACGGAGGGCGACUCGUGUAU | 10095 | AACGGAGGGCGACTCGTGTAT |
| 4431 | AACGGGGACAUCAUUCUCCCA | 10096 | AACGGGGACATCATTCTCCCA |
| 4432 | AACGUCGCCCAGAGCUUGGUG | 10097 | AACGTCGCCCAGAGCTTGGTG |
| 4433 | AACGUUUCAACCUCGUAAGGA | 10098 | AACGTTTCAACCTCGTAAGGA |
| 4434 | AACGUUUUCCUCUUCACGCCC | 10099 | AACGTTTTCCTCTTCACGCCC |
| 4435 | AACUCAAAACUUCCUGGAGAG | 10100 | AACTCAAAACTTCCTGGAGAG |
| 4436 | AACUCAAGUGAUCCAGCUCAG | 10101 | AACTCAAGTGATCCAGCTCAG |
| 4437 | AACUCCUGAACUCAAGUGAUC | 10102 | AACTCCTGAACTCAAGTGATC |
| 4438 | AACUCGAAGGCCGAGCAGGGG | 10103 | AACTCGAAGGCCGAGCAGGGG |
| 4439 | AACUCGUCCUGGGAGCACGUC | 10104 | AACTCGTCCTGGGAGCACGTC |
| 4440 | AACUCGUUUCUUUCGCAUCUG | 10105 | AACTCGTTTCTTTCGCATCTG |
| 4441 | AACUCUGAACUGAGAAAGUGC | 10106 | AACTCTGAACTGAGAAAGTGC |
| 4442 | AACUGAGAAAGUGCAAGGAGA | 10107 | AACTGAGAAAGTGCAAGGAGA |
| 4443 | AACUGAGGAAUGCAGCGGUUG | 10108 | AACTGAGGAATGCAGCGGTTG |
| 4444 | AACUGCCGAGAGAUGCACUUC | 10109 | AACTGCCGAGAGATGCACTTC |
| 4445 | AACUUCAUCGCUCAUGUCCUU | 10110 | AACTTCATCGCTCATGTCCTT |
| 4446 | AACUUCCUGGAGAGAAAUGGA | 10111 | AACTTCCTGGAGAGAAATGGA |
| 4447 | AACUUGAUCGGGGCUGGUGGG | 10112 | AACTTGATCGGGGCTGGTGGG |
| 4448 | AACUUGGGCGAGUGGGGGUUG | 10113 | AACTTGGGCGAGTGGGGGTTG |
| 4449 | AACUUGUUGGGUCCCUCGCAG | 10114 | AACTTGTTGGGTCCCTCGCAG |
| 4450 | AACUUUUUGGUUUUUGUAGAG | 10115 | AACTTTTTGGTTTTTGTAGAG |
| 4451 | AAGAAACCAAAAUCCCAACCC | 10116 | AAGAAACCAAAATCCCAACCC |
| 4452 | AAGAAAUUUAAUUGAUCACAG | 10117 | AAGAAATTTAATTGATCACAG |
| 4453 | AAGAACCUGAAGUCCCGUCAA | 10118 | AAGAACCTGAAGTCCCGTCAA |
| 4454 | AAGAAGAGGCAUUUCAGAGGC | 10119 | AAGAAGAGGCATTTCAGAGGC |
| 4455 | AAGAAGAGGUAGGCGAUGGAG | 10120 | AAGAAGAGGTAGGCGATGGAG |
| 4456 | AAGACACUGGGUGGGUCAUGA | 10121 | AAGACACTGGGTGGGTCATGA |
| 4457 | AAGACAUUCAGGCUACAAAAA | 10122 | AAGACATTCAGGCTACAAAAA |
| 4458 | AAGACCCCCAGGCAAAGGAAG | 10123 | AAGACCCCCAGGCAAAGGAAG |
| 4459 | AAGACCCCUACAGCGCUGCGG | 10124 | AAGACCCCTACAGCGCTGCGG |
| 4460 | AAGACGACAAUGGCAGUUCUC | 10125 | AAGACGACAATGGCAGTTCTC |
| 4461 | AAGACGAGGAGCACGAUGGGG | 10126 | AAGACGAGGAGCACGATGGGG |
| 4462 | AAGACGAGGAGCACUGUAGGG | 10127 | AAGACGAGGAGCACTGTAGGG |
| 4463 | AAGACGGCCAAGGAGAAGGGG | 10128 | AAGACGGCCAAGGAGAAGGGG |
| 4464 | AAGAGAACCAUAUCCUCUGGG | 10129 | AAGAGAACCATATCCTCTGGG |
| 4465 | AAGAGACGCCGUGGGCUCUGU | 10130 | AAGAGACGCCGTGGGCTCTGT |
| 4466 | AAGAGAUGGGGUCUUACUAUG | 10131 | AAGAGATGGGGTCTTACTATG |
| 4467 | AAGAGGCAUUUCAGAGGCAAU | 10132 | AAGAGGCATTTCAGAGGCAAT |
| 4468 | AAGAGGUAAACACGUUGACUC | 10133 | AAGAGGTAAACACGTTGACTC |
| 4469 | AAGAGGUAGGCGAUGGAGCCC | 10134 | AAGAGGTAGGCGATGGAGCCC |
| 4470 | AAGAUCUCGGCUGCAGACCAA | 10135 | AAGATCTCGGCTGCAGACCAA |
| 4471 | AAGAUGGUCUUCCGGUUGCCC | 10136 | AAGATGGTCTTCCGGTTGCCC |
| 4472 | AAGAUUGGGGAAGUGAAUGGC | 10137 | AAGATTGGGGAAGTGAATGGC |
| 4473 | AAGCACGUCUCCUGGGACUCA | 10138 | AAGCACGTCTCCTGGGACTCA |
| 4474 | AAGCACUAGGUGGGCGGACAU | 10139 | AAGCACTAGGTGGGCGGACAT |
| 4475 | AAGCAGUCCCGGUCUGAGUCA | 10140 | AAGCAGTCCCGGTCTGAGTCA |
| 4476 | AAGCAGUGGCAUAAUCAUAGC | 10141 | AAGCAGTGGCATAATCATAGC |
| 4477 | AAGCAUUCGUUGGUCCCGCAC | 10142 | AAGCATTCGTTGGTCCCGCAC |
| 4478 | AAGCCACUCAUACAUACAACG | 10143 | AAGCCACTCATACATACAACG |
| 4479 | AAGCCACUUGCUUCUCUGGGC | 10144 | AAGCCACTTGCTTCTCTGGGC |
| 4480 | AAGCCAUGAACAGGAUCCACC | 10145 | AAGCCATGAACAGGATCCACC |
| 4481 | AAGCCAUUGCAUGGGCACUGU | 10146 | AAGCCATTGCATGGGCACTGT |
| 4482 | AAGCCGCCAGUUCUUCCAUAG | 10147 | AAGCCGCCAGTTCTTCCATAG |
| 4483 | AAGCCGUCGGGGCACAGGCAC | 10148 | AAGCCGTCGGGGCACAGGCAC |
| 4484 | AAGCCUGUUCUGCCUCCCAGA | 10149 | AAGCCTGTTCTGCCTCCCAGA |
| 4485 | AAGCCUUCCUCACACUGGCAC | 10150 | AAGCCTTCCTCACACTGGCAC |
| 4486 | AAGCGUUGCACACAGUCCAGU | 10151 | AAGCGTTGCACACAGTCCAGT |
| 4487 | AAGCUCCGCUUCCCGGGUUCA | 10152 | AAGCTCCGCTTCCCGGGTTCA |
| 4488 | AAGCUCUGGCAGGCAAUGCUU | 10153 | AAGCTCTGGCAGGCAATGCTT |
| 4489 | AAGCUGGCGGGACCACAGGUG | 10154 | AAGCTGGCGGGACCACAGGTG |
| 4490 | AAGCUGGGUGCUGCAGAUCAU | 10155 | AAGCTGGGTGCTGCAGATCAT |
| 4491 | AAGGAACCGAGGCCUUGAGGG | 10156 | AAGGAACCGAGGCCTTGAGGG |
| 4492 | AAGGAAGAAACCAAAAUCCCA | 10157 | AAGGAAGAAACCAAAATCCCA |
| 4493 | AAGGAAGACCCCCAGGCAAAG | 10158 | AAGGAAGACCCCCAGGCAAAG |
| 4494 | AAGGAAGACGAGGAGCACGAU | 10159 | AAGGAAGACGAGGAGCACGAT |
| 4495 | AAGGAAGACGAGGAGCACUGU | 10160 | AAGGAAGACGAGGAGCACTGT |
| 4496 | AAGGAAGGUACAAACCCACCU | 10161 | AAGGAAGGTACAAACCCACCT |
| 4497 | AAGGAGAAGGGGUGGGCCAGC | 10162 | AAGGAGAAGGGGTGGGCCAGC |
| 4498 | AAGGAGACCACGGGAAUGGAA | 10163 | AAGGAGACCACGGGAATGGAA |
| 4499 | AAGGAGGGGGGGGGAGAUUCU | 10164 | AAGGAGGGGGGGGGAGATTCT |
| 4500 | AAGGCCGAGCAGGGGCUACUG | 10165 | AAGGCCGAGCAGGGGCTACTG |
| 4501 | AAGGCCGGCGAGGUCUCAGGA | 10166 | AAGGCCGGCGAGGTCTCAGGA |
| 4502 | AAGGCGACGGUCCAGCGCAAU | 10167 | AAGGCGACGGTCCAGCGCAAT |
| 4503 | AAGGCUAACCUGGCUGUCUAG | 10168 | AAGGCTAACCTGGCTGTCTAG |
| 4504 | AAGGCUUAUCACGGUUCCCUG | 10169 | AAGGCTTATCACGGTTCCCTG |
| 4505 | AAGGCUUGCAUAGAAGAGGUA | 10170 | AAGGCTTGCATAGAAGAGGTA |
| 4506 | AAGGGGUGGGCCAGCCUCUUU | 10171 | AAGGGGTGGGCCAGCCTCTTT |
| 4507 | AAGGGUGACCAGUGACUCAGG | 10172 | AAGGGTGACCAGTGACTCAGG |
| 4508 | AAGGGUUCUGGGCAGGGACGG | 10173 | AAGGGTTCTGGGCAGGGACGG |
| 4509 | AAGGUAACCGGGUGUCUCAGG | 10174 | AAGGTAACCGGGTGTCTCAGG |
| 4510 | AAGGUACAAACCCACCUCGAA | 10175 | AAGGTACAAACCCACCTCGAA |
| 4511 | AAGGUCAUUGCAGACGUGGGA | 10176 | AAGGTCATTGCAGACGTGGGA |
| 4512 | AAGGUGGGGACGGAGGUGUCG | 10177 | AAGGTGGGGACGGAGGTGTCG |
| 4513 | AAGUAGCUGGGAUCACAGGCA | 10178 | AAGTAGCTGGGATCACAGGCA |
| 4514 | AAGUCCCCGGAUUUGCAGGUG | 10179 | AAGTCCCCGGATTTGCAGGTG |
| 4515 | AAGUCCCGGUCAACCUGCCCC | 10180 | AAGTCCCGGTCAACCTGCCCC |
| 4516 | AAGUCCCGUCAAACGAUCCAG | 10181 | AAGTCCCGTCAAACGATCCAG |
| 4517 | AAGUGAAUGGCUUGGAGGCAU | 10182 | AAGTGAATGGCTTGGAGGCAT |
| 4518 | AAGUGACACCCAUCUCCCAGA | 10183 | AAGTGACACCCATCTCCCAGA |
| 4519 | AAGUGAUAACAGUUUCAACCA | 10184 | AAGTGATAACAGTTTCAACCA |
| 4520 | AAGUGAUCCAGCUCAGCCUCC | 10185 | AAGTGATCCAGCTCAGCCTCC |
| 4521 | AAGUGCAAACAUAACAAAAAA | 10186 | AAGTGCAAACATAACAAAAAA |
| 4522 | AAGUGCAAGGAGACCACGGGA | 10187 | AAGTGCAAGGAGACCACGGGA |
| 4523 | AAGUGCUGGGAUUACAGGCGU | 10188 | AAGTGCTGGGATTACAGGCGT |
| 4524 | AAGUGGAAGCACUAGGUGGGC | 10189 | AAGTGGAAGCACTAGGTGGGC |
| 4525 | AAGUGGCAUCAUUUGGUGAAU | 10190 | AAGTGGCATCATTTGGTGAAT |
| 4526 | AAGUGGGUAGGGGUCGGGAGG | 10191 | AAGTGGGTAGGGGTCGGGAGG |
| 4527 | AAGUUGACAUCGGAACCUGUG | 10192 | AAGTTGACATCGGAACCTGTG |
| 4528 | AAGUUGAUGCUGUUGAUGUUC | 10193 | AAGTTGATGCTGTTGATGTTC |
| 4529 | AAGUUUGGAGUCAACCCAGUA | 10194 | AAGTTTGGAGTCAACCCAGTA |
| 4530 | AAUAAAACAAGGCCGGCGAGG | 10195 | AATAAAACAAGGCCGGCGAGG |
| 4531 | AAUAAAUAGAUUUUAUAUAUA | 10196 | AATAAATAGATTTTATATATA |
| 4532 | AAUAAAUAUAUAAAACAAAGC | 10197 | AATAAATATATAAAACAAAGC |
| 4533 | AAUAAAUAUUAAGGGUGACCA | 10198 | AATAAATATTAAGGGTGACCA |
| 4534 | AAUAACACAAAUGCCAAAUGU | 10199 | AATAACACAAATGCCAAATGT |
| 4535 | AAUAACCCCCUACACAGGGCC | 10200 | AATAACCCCCTACACAGGGCC |
| 4536 | AAUAACGUUUUCCUCUUCACG | 10201 | AATAACGTTTTCCTCTTCACG |
| 4537 | AAUAAUAACACAAAUGCCAAA | 10202 | AATAATAACACAAATGCCAAA |
| 4538 | AAUAAUCUUUGUACAUAAAGA | 10203 | AATAATCTTTGTACATAAAGA |
| 4539 | AAUACAGCAACCAGGGUUUGC | 10204 | AATACAGCAACCAGGGTTTGC |
| 4540 | AAUACUUUGUCCUCAAAGACG | 10205 | AATACTTTGTCCTCAAAGACG |
| 4541 | AAUAGAUUUUAUAUAUAUGUG | 10206 | AATAGATTTTATATATATGTG |
| 4542 | AAUAGGGUCUUCAUCUGUUGC | 10207 | AATAGGGTCTTCATCTGTTGC |
| 4543 | AAUAGUCACUGAACAAAUACA | 10208 | AATAGTCACTGAACAAATACA |
| 4544 | AAUAUACAAAGUGCAAACAUA | 10209 | AATATACAAAGTGCAAACATA |
| 4545 | AAUAUAUAAAACAAAGCUCUG | 10210 | AATATATAAAACAAAGCTCTG |
| 4546 | AAUAUUAAGGGUGACCAGUGA | 10211 | AATATTAAGGGTGACCAGTGA |
| 4547 | AAUCAUAGCUCACGACAGCCU | 10212 | AATCATAGCTCACGACAGCCT |
| 4548 | AAUCCCAACCCAAGCCAUUGC | 10213 | AATCCCAACCCAAGCCATTGC |
| 4549 | AAUCCCCACUAUCUGUACAGG | 10214 | AATCCCCACTATCTGTACAGG |
| 4550 | AAUCCCUUGUGACAUCUUCAC | 10215 | AATCCCTTGTGACATCTTCAC |
| 4551 | AAUCCCUUUCCUGGCUUAAGG | 10216 | AATCCCTTTCCTGGCTTAAGG |
| 4552 | AAUCUCGGCUCACUGCAAGCU | 10217 | AATCTCGGCTCACTGCAAGCT |
| 4553 | AAUCUUUGUACAUAAAGAAGA | 10218 | AATCTTTGTACATAAAGAAGA |
| 4554 | AAUGAGUUUGGUCUCGGUGCC | 10219 | AATGAGTTTGGTCTCGGTGCC |
| 4555 | AAUGCAGCGGUUGACACGGCC | 10220 | AATGCAGCGGTTGACACGGCC |
| 4556 | AAUGCCAAAUGUACACAGUGU | 10221 | AATGCCAAATGTACACAGTGT |
| 4557 | AAUGCCUGAGCUCAAACCAUC | 10222 | AATGCCTGAGCTCAAACCATC |
| 4558 | AAUGCUUUGGUCUUCUCUGUC | 10223 | AATGCTTTGGTCTTCTCTGTC |
| 4559 | AAUGGAAGUGGGUAGGGGUCG | 10224 | AATGGAAGTGGGTAGGGGTCG |
| 4560 | AAUGGACAGAGCCCUCACGCU | 10225 | AATGGACAGAGCCCTCACGCT |
| 4561 | AAUGGAGGUGUCAUCCUGGUC | 10226 | AATGGAGGTGTCATCCTGGTC |
| 4562 | AAUGGCAGUUCUCGAUAUUCA | 10227 | AATGGCAGTTCTCGATATTCA |
| 4563 | AAUGGCUUCGUUGAUGAUAUC | 10228 | AATGGCTTCGTTGATGATATC |
| 4564 | AAUGGCUUGGAGGCAUUUGCA | 10229 | AATGGCTTGGAGGCATTTGCA |
| 4565 | AAUGUACACAGUGUACAACUC | 10230 | AATGTACACAGTGTACAACTC |
| 4566 | AAUGUGGACCUCAUCCUCUGU | 10231 | AATGTGGACCTCATCCTCTGT |
| 4567 | AAUGUUUUCAGUCACCAGCGA | 10232 | AATGTTTTCAGTCACCAGCGA |
| 4568 | AAUUCAUUUUUUAAGAGAUGG | 10233 | AATTCATTTTTTAAGAGATGG |
| 4569 | AAUUCGUCAGGGCGACAGGUG | 10234 | AATTCGTCAGGGCGACAGGTG |
| 4570 | AAUUGAUCACAGUAAGACAUU | 10235 | AATTGATCACAGTAAGACATT |
| 4571 | AAUUGCCAAUCCCUUGUGACA | 10236 | AATTGCCAATCCCTTGTGACA |
| 4572 | AAUUUAAUUGAUCACAGUAAG | 10237 | AATTTAATTGATCACAGTAAG |
| 4573 | AAUUUCCAGCCCCAGGGCCCC | 10238 | AATTTCCAGCCCCAGGGCCCC |
| 4574 | AAUUUUUUGUAUUUUUAGUAG | 10239 | AATTTTTTGTATTTTTAGTAG |
| 4575 | ACAAAAAAUCCCCACUAUCUG | 10240 | ACAAAAAATCCCCACTATCTG |
| 4576 | ACAAAAACCUUCUAUACAGUG | 10241 | ACAAAAACCTTCTATACAGTG |
| 4577 | ACAAACCCACCUCGAAAGACA | 10242 | ACAAACCCACCTCGAAAGACA |
| 4578 | ACAAAGCUCUGGCAGGCAAUG | 10243 | ACAAAGCTCTGGCAGGCAATG |
| 4579 | ACAAAGUGCAAACAUAACAAA | 10244 | ACAAAGTGCAAACATAACAAA |
| 4580 | ACAAAUACAGCAACCAGGGUU | 10245 | ACAAATACAGCAACCAGGGTT |
| 4581 | ACAAAUGCCAAAUGUACACAG | 10246 | ACAAATGCCAAATGTACACAG |
| 4582 | ACAACGGGGACAUCAUUCUCC | 10247 | ACAACGGGGACATCATTCTCC |
| 4583 | ACAACUCUGAACUGAGAAAGU | 10248 | ACAACTCTGAACTGAGAAAGT |
| 4584 | ACAAGAUUGGGGAAGUGAAUG | 10249 | ACAAGATTGGGGAAGTGAATG |
| 4585 | ACAAGCACGUCUCCUGGGACU | 10250 | ACAAGCACGTCTCCTGGGACT |
| 4586 | ACAAGGCCGGCGAGGUCUCAG | 10251 | ACAAGGCCGGCGAGGTCTCAG |
| 4587 | ACAAGGUGGGGACGGAGGUGU | 10252 | ACAAGGTGGGGACGGAGGTGT |
| 4588 | ACAAGUUGACAUCGGAACCUG | 10253 | ACAAGTTGACATCGGAACCTG |
| 4589 | ACAAUGGACAGAGCCCUCACG | 10254 | ACAATGGACAGAGCCCTCACG |
| 4590 | ACAAUGGCAGUUCUCGAUAUU | 10255 | ACAATGGCAGTTCTCGATATT |
| 4591 | ACAAUUGCCAAUCCCUUGUGA | 10256 | ACAATTGCCAATCCCTTGTGA |
| 4592 | ACACAAAUGCCAAAUGUACAC | 10257 | ACACAAATGCCAAATGTACAC |
| 4593 | ACACACACGACAGAAAACAGU | 10258 | ACACACACGACAGAAAACAGT |
| 4594 | ACACACGACAGAAAACAGUGC | 10259 | ACACACGACAGAAAACAGTGC |
| 4595 | ACACAGCUGCCCCCGAGACCA | 10260 | ACACAGCTGCCCCCGAGACCA |
| 4596 | ACACAGGGCCCCGAGAAUAGU | 10261 | ACACAGGGCCCCGAGAATAGT |
| 4597 | ACACAGUCCAGUUCGUGCAAA | 10262 | ACACAGTCCAGTTCGTGCAAA |
| 4598 | ACACAGUGUACAACUCUGAAC | 10263 | ACACAGTGTACAACTCTGAAC |
| 4599 | ACACAGUGUUUUGUCCCUGGG | 10264 | ACACAGTGTTTTGTCCCTGGG |
| 4600 | ACACAUACCCAUCAACGACAA | 10265 | ACACATACCCATCAACGACAA |
| 4601 | ACACCAGUUCACUCCUCUUGG | 10266 | ACACCAGTTCACTCCTCTTGG |
| 4602 | ACACCAUUCAGGCCCCCUUUC | 10267 | ACACCATTCAGGCCCCCTTTC |
| 4603 | ACACCAUUCUCCUGCCUCAGC | 10268 | ACACCATTCTCCTGCCTCAGC |
| 4604 | ACACCCAUCUCCCAGAAGCCA | 10269 | ACACCCATCTCCCAGAAGCCA |
| 4605 | ACACCUGGGUGCAGGCCACGU | 10270 | ACACCTGGGTGCAGGCCACGT |
| 4606 | ACACGACAGAAAACAGUGCAA | 10271 | ACACGACAGAAAACAGTGCAA |
| 4607 | ACACGGCCCCCACAGCUGAAG | 10272 | ACACGGCCCCCACAGCTGAAG |
| 4608 | ACACGGCGAUUUCAGAAAGAA | 10273 | ACACGGCGATTTCAGAAAGAA |
| 4609 | ACACGGGCUUUCCCUGGCCUG | 10274 | ACACGGGCTTTCCCTGGCCTG |
| 4610 | ACACGUAAAGACCCCUACAGC | 10275 | ACACGTAAAGACCCCTACAGC |
| 4611 | ACACGUUGACUCCUGAGUUUA | 10276 | ACACGTTGACTCCTGAGTTTA |
| 4612 | ACACUCAUCGAUAUCGCACUC | 10277 | ACACTCATCGATATCGCACTC |
| 4613 | ACACUCAUCGAUAUCUUCGCA | 10278 | ACACTCATCGATATCTTCGCA |
| 4614 | ACACUGCCGGCUGCCAUGGAU | 10279 | ACACTGCCGGCTGCCATGGAT |
| 4615 | ACACUGGCACUUGUAGCCACC | 10280 | ACACTGGCACTTGTAGCCACC |
| 4616 | ACACUGGGUGGGUCAUGAUUC | 10281 | ACACTGGGTGGGTCATGATTC |
| 4617 | ACACUUGAACUUGUUGGGUCC | 10282 | ACACTTGAACTTGTTGGGTCC |
| 4618 | ACAGAAAACAGUGCAAAAUAA | 10283 | ACAGAAAACAGTGCAAAATAA |
| 4619 | ACAGACAAGCACGUCUCCUGG | 10284 | ACAGACAAGCACGTCTCCTGG |
| 4620 | ACAGACGAACUGCCGAGAGAU | 10285 | ACAGACGAACTGCCGAGAGAT |
| 4621 | ACAGAGACAGUGCCCAGGACA | 10286 | ACAGAGACAGTGCCCAGGACA |
| 4622 | ACAGAGCACAGCGGAAACCGG | 10287 | ACAGAGCACAGCGGAAACCGG |
| 4623 | ACAGAGCCCUCACGCUACUGG | 10288 | ACAGAGCCCTCACGCTACTGG |
| 4624 | ACAGAGUCGGUCCAGUAGAUG | 10289 | ACAGAGTCGGTCCAGTAGATG |
| 4625 | ACAGAUACUGGCAGCCGCCAU | 10290 | ACAGATACTGGCAGCCGCCAT |
| 4626 | ACAGCAACCAGGGUUUGCAAA | 10291 | ACAGCAACCAGGGTTTGCAAA |
| 4627 | ACAGCCACACCUGGGUGCAGG | 10292 | ACAGCCACACCTGGGTGCAGG |
| 4628 | ACAGCCAGCCCGUCGGGGGCC | 10293 | ACAGCCAGCCCGTCGGGGGCC |
| 4629 | ACAGCCGCCGUUGUUGUCCAA | 10294 | ACAGCCGCCGTTGTTGTCCAA |
| 4630 | ACAGCCUCAAAUGCCUGAGCU | 10295 | ACAGCCTCAAATGCCTGAGCT |
| 4631 | ACAGCCUUGCAGGCCUUCGUG | 10296 | ACAGCCTTGCAGGCCTTCGTG |
| 4632 | ACAGCCUUGCUCGUCUGAGCC | 10297 | ACAGCCTTGCTCGTCTGAGCC |
| 4633 | ACAGCGCAGUUUUCCUCGUCA | 10298 | ACAGCGCAGTTTTCCTCGTCA |
| 4634 | ACAGCGCCAGCUGGAGUGGAU | 10299 | ACAGCGCCAGCTGGAGTGGAT |
| 4635 | ACAGCGCUGCGGCCACUCAUC | 10300 | ACAGCGCTGCGGCCACTCATC |
| 4636 | ACAGCGGAAACCGGGGACUUC | 10301 | ACAGCGGAAACCGGGGACTTC |
| 4637 | ACAGCUGAAGUCCCCGGAUUU | 10302 | ACAGCTGAAGTCCCCGGATTT |
| 4638 | ACAGCUGCCCCCGAGACCAGA | 10303 | ACAGCTGCCCCCGAGACCAGA |
| 4639 | ACAGCUGGGGGAUGCAGGUGG | 10304 | ACAGCTGGGGGATGCAGGTGG |
| 4640 | ACAGGAACCGCGGAGGAACGC | 10305 | ACAGGAACCGCGGAGGAACGC |
| 4641 | ACAGGACAGCCUUGCUCGUCU | 10306 | ACAGGACAGCCTTGCTCGTCT |
| 4642 | ACAGGAUCCACCACGAUGGCC | 10307 | ACAGGATCCACCACGATGGCC |
| 4643 | ACAGGCACGUGCCACCGUACC | 10308 | ACAGGCACGTGCCACCGTACC |
| 4644 | ACAGGCACUCGUAGCCGAUCU | 10309 | ACAGGCACTCGTAGCCGATCT |
| 4645 | ACAGGCGUGAGCCACUGCGCC | 10310 | ACAGGCGTGAGCCACTGCGCC |
| 4646 | ACAGGCGUGAGGCACCUGUGU | 10311 | ACAGGCGTGAGGCACCTGTGT |
| 4647 | ACAGGGACGCAUUUACGUGCU | 10312 | ACAGGGACGCATTTACGTGCT |
| 4648 | ACAGGGCAAAGGCUAACCUGG | 10313 | ACAGGGCAAAGGCTAACCTGG |
| 4649 | ACAGGGCCCCGAGAAUAGUCA | 10314 | ACAGGGCCCCGAGAATAGTCA |
| 4650 | ACAGGUCAGACCAGUAGAUUC | 10315 | ACAGGTCAGACCAGTAGATTC |
| 4651 | ACAGGUCGGGUGGUUGUGUGC | 10316 | ACAGGTCGGGTGGTTGTGTGC |
| 4652 | ACAGGUGAGCACCGGGCAGGA | 10317 | ACAGGTGAGCACCGGGCAGGA |
| 4653 | ACAGGUGCCCGCCACCACGCC | 10318 | ACAGGTGCCCGCCACCACGCC |
| 4654 | ACAGGUGGCCACAGCGCAGUU | 10319 | ACAGGTGGCCACAGCGCAGTT |
| 4655 | ACAGGUGGCCACAGGACAGCC | 10320 | ACAGGTGGCCACAGGACAGCC |
| 4656 | ACAGUAACACGGCGAUUUCAG | 10321 | ACAGTAACACGGCGATTTCAG |
| 4657 | ACAGUAAGACAUUCAGGCUAC | 10322 | ACAGTAAGACATTCAGGCTAC |
| 4658 | ACAGUAGGUUUUCAGCCAACA | 10323 | ACAGTAGGTTTTCAGCCAACA |
| 4659 | ACAGUCCAGUUCGUGCAAAUA | 10324 | ACAGTCCAGTTCGTGCAAATA |
| 4660 | ACAGUGCAAAAUAAUAACACA | 10325 | ACAGTGCAAAATAATAACACA |
| 4661 | ACAGUGCCCAGGACAGAGUCG | 10326 | ACAGTGCCCAGGACAGAGTCG |
| 4662 | ACAGUGGUUUAAAAAGUGACA | 10327 | ACAGTGGTTTAAAAAGTGACA |
| 4663 | ACAGUGUACAACUCUGAACUG | 10328 | ACAGTGTACAACTCTGAACTG |
| 4664 | ACAGUGUUUUGUCCCUGGGGA | 10329 | ACAGTGTTTTGTCCCTGGGGA |
| 4665 | ACAGUUUCAACCAAUAUACAA | 10330 | ACAGTTTCAACCAATATACAA |
| 4666 | ACAUAAAGAAGAGGCAUUUCA | 10331 | ACATAAAGAAGAGGCATTTCA |
| 4667 | ACAUAAAGACGACAAUGGCAG | 10332 | ACATAAAGACGACAATGGCAG |
| 4668 | ACAUAACAAAAAAUCCCCACU | 10333 | ACATAACAAAAAATCCCCACT |
| 4669 | ACAUACAACGGGGACAUCAUU | 10334 | ACATACAACGGGGACATCATT |
| 4670 | ACAUACCCAUCAACGACAAGA | 10335 | ACATACCCATCAACGACAAGA |
| 4671 | ACAUCAGUGCAACAGUAACAC | 10336 | ACATCAGTGCAACAGTAACAC |
| 4672 | ACAUCAUUCUCCCAAAAAGCG | 10337 | ACATCATTCTCCCAAAAAGCG |
| 4673 | ACAUCCCCUCCCCUUUCAUCU | 10338 | ACATCCCCTCCCCTTTCATCT |
| 4674 | ACAUCGAUGCUUGAGAUGGAG | 10339 | ACATCGATGCTTGAGATGGAG |
| 4675 | ACAUCGGAACCUGUGAGGCGG | 10340 | ACATCGGAACCTGTGAGGCGG |
| 4676 | ACAUCUUCACGCGGGAGUCUG | 10341 | ACATCTTCACGCGGGAGTCTG |
| 4677 | ACAUGAAGCCAUGAACAGGAU | 10342 | ACATGAAGCCATGAACAGGAT |
| 4678 | ACAUUAACGCAGCCAACUUCA | 10343 | ACATTAACGCAGCCAACTTCA |
| 4679 | ACAUUCAGGCUACAAAAACCU | 10344 | ACATTCAGGCTACAAAAACCT |
| 4680 | ACAUUGUCACUAUCUCCACCG | 10345 | ACATTGTCACTATCTCCACCG |
| 4681 | ACCAAAAUCCCAACCCAAGCC | 10346 | ACCAAAATCCCAACCCAAGCC |
| 4682 | ACCAAGGAGGGGGGGGAGAU | 10347 | ACCAAGGAGGGGCGGGGAGAT |
| 4683 | ACCAAUAUACAAAGUGCAAAC | 10348 | ACCAATATACAAAGTGCAAAC |
| 4684 | ACCACAGGUGAGCACCGGGCA | 10349 | ACCACAGGTGAGCACCGGGCA |
| 4685 | ACCACGAUGGCCCUUGGCUUG | 10350 | ACCACGATGGCCCTTGGCTTG |
| 4686 | ACCACGCCCGGCUAAUUUUUU | 10351 | ACCACGCCCGGCTAATTTTTT |
| 4687 | ACCACGGAUUCAGCCAGAUCA | 10352 | ACCACGGATTCAGCCAGATCA |
| 4688 | ACCACGGGAAUGGAAGUGGGU | 10353 | ACCACGGGAATGGAAGTGGGT |
| 4689 | ACCACGUUCCUCAGGUUGGGG | 10354 | ACCACGTTCCTCAGGTTGGGG |
| 4690 | ACCAGAAAGGCUUAUCACGGU | 10355 | ACCAGAAAGGCTTATCACGGT |
| 4691 | ACCAGAGACCCAGCUCCGUGG | 10356 | ACCAGAGACCCAGCTCCGTGG |
| 4692 | ACCAGCGAGUAGAUGUCCACA | 10357 | ACCAGCGAGTAGATGTCCACA |
| 4693 | ACCAGCUGGAAGCCGUCGGGG | 10358 | ACCAGCTGGAAGCCGTCGGGG |
| 4694 | ACCAGGCUGGUGUCCUGACAG | 10359 | ACCAGGCTGGTGTCCTGACAG |
| 4695 | ACCAGGGUUUGCAAAAAUAAA | 10360 | ACCAGGGTTTGCAAAAATAAA |
| 4696 | ACCAGUAGAUUCUAUUGCUGG | 10361 | ACCAGTAGATTCTATTGCTGG |
| 4697 | ACCAGUCCCGGCAGUCUCUAG | 10362 | ACCAGTCCCGGCAGTCTCTAG |
| 4698 | ACCAGUGACUCAGGCUCUGCC | 10363 | ACCAGTGACTCAGGCTCTGCC |
| 4699 | ACCAGUUCACUCCUCUUGGCU | 10364 | ACCAGTTCACTCCTCTTGGCT |
| 4700 | ACCAUAUCCUCUGGGGACAGU | 10365 | ACCATATCCTCTGGGGACAGT |
| 4701 | ACCAUCACAGCGCCAGCUGGA | 10366 | ACCATCACAGCGCCAGCTGGA |
| 4702 | ACCAUCCUCCCACCUCAGCCU | 10367 | ACCATCCTCCCACCTCAGCCT |
| 4703 | ACCAUCGAGGGGUAGCUGUAG | 10368 | ACCATCGAGGGGTAGCTGTAG |
| 4704 | ACCAUCUGUCUCGAGGGGUAG | 10369 | ACCATCTGTCTCGAGGGGTAG |
| 4705 | ACCAUGCAUGGUUUUUUUUUG | 10370 | ACCATGCATGGTTTTTTTTTG |
| 4706 | ACCAUUCAGGCCCCCUUUCUU | 10371 | ACCATTCAGGCCCCCTTTCTT |
| 4707 | ACCAUUCUCCUGCCUCAGCCU | 10372 | ACCATTCTCCTGCCTCAGCCT |
| 4708 | ACCCAAGCCAUUGCAUGGGCA | 10373 | ACCCAAGCCATTGCATGGGCA |
| 4709 | ACCCACCUCGAAAGACACUGG | 10374 | ACCCACCTCGAAAGACACTGG |
| 4710 | ACCCACUUGUAGGAGAUGCAU | 10375 | ACCCACTTGTAGGAGATGCAT |
| 4711 | ACCCAGCUCCGUGGCGAUGGG | 10376 | ACCCAGCTCCGTGGCGATGGG |
| 4712 | ACCCAGCUGAUUUUUAACUUU | 10377 | ACCCAGCTGATTTTTAACTTT |
| 4713 | ACCCAGUAGAGGCGGCCACUG | 10378 | ACCCAGTAGAGGCGGCCACTG |
| 4714 | ACCCAUCAACGACAAGAUUGG | 10379 | ACCCATCAACGACAAGATTGG |
| 4715 | ACCCAUCUCCCAGAAGCCACU | 10380 | ACCCATCTCCCAGAAGCCACT |
| 4716 | ACCCCCAGGCAAAGGAAGACG | 10381 | ACCCCCAGGCAAAGGAAGACG |
| 4717 | ACCCCCUACACAGGGCCCCGA | 10382 | ACCCCCTACACAGGGCCCCGA |
| 4718 | ACCCCUACAGCGCUGCGGCCA | 10383 | ACCCCTACAGCGCTGCGGCCA |
| 4719 | ACCCGCCUCGGCCUCCCAAAG | 10384 | ACCCGCCTCGGCCTCCCAAAG |
| 4720 | ACCCGGAUCACGACCUGCUGU | 10385 | ACCCGGATCACGACCTGCTGT |
| 4721 | ACCCGGGACUUGUCGUCGCCU | 10386 | ACCCGGGACTTGTCGTCGCCT |
| 4722 | ACCCUCCAGGUUCACGCAGAG | 10387 | ACCCTCCAGGTTCACGCAGAG |
| 4723 | ACCCUGGCUUCCCGCGAUUGC | 10388 | ACCCTGGCTTCCCGCGATTGC |
| 4724 | ACCGAGGCCUUGAGGGUGGCC | 10389 | ACCGAGGCCTTGAGGGTGGCC |
| 4725 | ACCGCGGAGGAACGCCAGGCC | 10390 | ACCGCGGAGGAACGCCAGGCC |
| 4726 | ACCGGGCAGGAGGCCUCGUCU | 10391 | ACCGGGCAGGAGGCCTCGTCT |
| 4727 | ACCGGGGACUUCGCCAGGAGG | 10392 | ACCGGGGACTTCGCCAGGAGG |
| 4728 | ACCGGGUGUCUCAGGCACUUA | 10393 | ACCGGGTGTCTCAGGCACTTA |
| 4729 | ACCGUACCCAGCUGAUUUUUA | 10394 | ACCGTACCCAGCTGATTTTTA |
| 4730 | ACCGUGCAUGUUUUAAACACA | 10395 | ACCGTGCATGTTTTAAACACA |
| 4731 | ACCGUGGUGAGCCCAGGGGUG | 10396 | ACCGTGGTGAGCCCAGGGGTG |
| 4732 | ACCUCAGCCUCGCCAUGCCUG | 10397 | ACCTCAGCCTCGCCATGCCTG |
| 4733 | ACCUCAUCCUCUGUGGUCUUC | 10398 | ACCTCATCCTCTGTGGTCTTC |
| 4734 | ACCUCAUGAUCCACCCGCCUC | 10399 | ACCTCATGATCCACCCGCCTC |
| 4735 | ACCUCCACCUCCUGGGCUCAG | 10400 | ACCTCCACCTCCTGGGCTCAG |
| 4736 | ACCUCCAGAACUGAGGAAUGC | 10401 | ACCTCCAGAACTGAGGAATGC |
| 4737 | ACCUCCGUGUCCAGAGCGACC | 10402 | ACCTCCGTGTCCAGAGCGACC |
| 4738 | ACCUCCUGGGCUCAGGCGAUC | 10403 | ACCTCCTGGGCTCAGGCGATC |
| 4739 | ACCUCGAAAGACACUGGGUGG | 10404 | ACCTCGAAAGACACTGGGTGG |
| 4740 | ACCUCGUAAGGAACCGAGGCC | 10405 | ACCTCGTAAGGAACCGAGGCC |
| 4741 | ACCUCGUGCCGGUUGGUGAAG | 10406 | ACCTCGTGCCGGTTGGTGAAG |
| 4742 | ACCUGAAGUCCCGUCAAACGA | 10407 | ACCTGAAGTCCCGTCAAACGA |
| 4743 | ACCUGCCCCUCUCUGUCCCCG | 10408 | ACCTGCCCCTCTCTGTCCCCG |
| 4744 | ACCUGCUGUGUCCUAGCUGGA | 10409 | ACCTGCTGTGTCCTAGCTGGA |
| 4745 | ACCUGGCUGUCUAGCAAGGCU | 10410 | ACCTGGCTGTCTAGCAAGGCT |
| 4746 | ACCUGGGUGCAGGCCACGUGU | 10411 | ACCTGGGTGCAGGCCACGTGT |
| 4747 | ACCUGUGAGGCGGUUGGCACU | 10412 | ACCTGTGAGGCGGTTGGCACT |
| 4748 | ACCUGUGUCUGGCCAAAUUCA | 10413 | ACCTGTGTCTGGCCAAATTCA |
| 4749 | ACCUUCUAUACAGUGGUUUAA | 10414 | ACCTTCTATACAGTGGTTTAA |
| 4750 | ACCUUUAGCCUGACGGUGGAU | 10415 | ACCTTTAGCCTGACGGTGGAT |
| 4751 | ACGAACUGCCGAGAGAUGCAC | 10416 | ACGAACTGCCGAGAGATGCAC |
| 4752 | ACGACAAGAUUGGGGAAGUGA | 10417 | ACGACAAGATTGGGGAAGTGA |
| 4753 | ACGACAAUGGCAGUUCUCGAU | 10418 | ACGACAATGGCAGTTCTCGAT |
| 4754 | ACGACAGAAAACAGUGCAAAA | 10419 | ACGACAGAAAACAGTGCAAAA |
| 4755 | ACGACAGCCUCAAAUGCCUGA | 10420 | ACGACAGCCTCAAATGCCTGA |
| 4756 | ACGACCUGCUGUGUCCUAGCU | 10421 | ACGACCTGCTGTGTCCTAGCT |
| 4757 | ACGAGGAAAGGAAGAAACCAA | 10422 | ACGAGGAAAGGAAGAAACCAA |
| 4758 | ACGAGGAGCACGAUGGGGAGG | 10423 | ACGAGGAGCACGATGGGGAGG |
| 4759 | ACGAGGAGCACUGUAGGGAGA | 10424 | ACGAGGAGCACTGTAGGGAGA |
| 4760 | ACGAUCACGGCUUUGAAGUCC | 10425 | ACGATCACGGCTTTGAAGTCC |
| 4761 | ACGAUCCAGACUGGAGGAUUU | 10426 | ACGATCCAGACTGGAGGATTT |
| 4762 | ACGAUGGCCCUUGGCUUGGAG | 10427 | ACGATGGCCCTTGGCTTGGAG |
| 4763 | ACGAUGGGGAGGACAAUGGAC | 10428 | ACGATGGGGAGGACAATGGAC |
| 4764 | ACGCAGAGCUGGCUGCAGGUG | 10429 | ACGCAGAGCTGGCTGCAGGTG |
| 4765 | ACGCAGCCAACUUCAUCGCUC | 10430 | ACGCAGCCAACTTCATCGCTC |
| 4766 | ACGCAUUUACGUGCUCCGAAA | 10431 | ACGCATTTACGTGCTCCGAAA |
| 4767 | ACGCCACGUCAUCCUCCAGAC | 10432 | ACGCCACGTCATCCTCCAGAC |
| 4768 | ACGCCAUGAAUCCCUUUCCUG | 10433 | ACGCCATGAATCCCTTTCCTG |
| 4769 | ACGCCCGGCUAAUUUUUUGUA | 10434 | ACGCCCGGCTAATTTTTTGTA |
| 4770 | ACGCCCUUGGUAUCCGCAACA | 10435 | ACGCCCTTGGTATCCGCAACA |
| 4771 | ACGCCGUGGGCUCUGUCAAGC | 10436 | ACGCCGTGGGCTCTGTCAAGC |
| 4772 | ACGCGGGAGUCUGACGGAGGC | 10437 | ACGCGGGAGTCTGACGGAGGC |
| 4773 | ACGCUACUGGGCUUCUUCUCA | 10438 | ACGCTACTGGGCTTCTTCTCA |
| 4774 | ACGCUCUGUCACACAGCUGCC | 10439 | ACGCTCTGTCACACAGCTGCC |
| 4775 | ACGGAGGCUGUCACUGUCUCU | 10440 | ACGGAGGCTGTCACTGTCTCT |
| 4776 | ACGGAGGGCGACUCGUGUAUG | 10441 | ACGGAGGGCGACTCGTGTATG |
| 4777 | ACGGAGGUGUCGCCACAGAGC | 10442 | ACGGAGGTGTCGCCACAGAGC |
| 4778 | ACGGAGUCUCGCUCUGUCGCC | 10443 | ACGGAGTCTCGCTCTGTCGCC |
| 4779 | ACGGAUUCAGCCAGAUCAUUU | 10444 | ACGGATTCAGCCAGATCATTT |
| 4780 | ACGGCCAAGGAGAAGGGGUGG | 10445 | ACGGCCAAGGAGAAGGGGTGG |
| 4781 | ACGGCCAAGGUAACCGGGUGU | 10446 | ACGGCCAAGGTAACCGGGTGT |
| 4782 | ACGGCCCCCACAGCUGAAGUC | 10447 | ACGGCCCCCACAGCTGAAGTC |
| 4783 | ACGGCGAUUUCAGAAAGAACC | 10448 | ACGGCGATTTCAGAAAGAACC |
| 4784 | ACGGCUGUGGAGCUGACCUUU | 10449 | ACGGCTGTGGAGCTGACCTTT |
| 4785 | ACGGCUUUGAAGUCCCGGUCA | 10450 | ACGGCTTTGAAGTCCCGGTCA |
| 4786 | ACGGGAAUGGAAGUGGGUAGG | 10451 | ACGGGAATGGAAGTGGGTAGG |
| 4787 | ACGGGACUCCAGGCAGAUGUU | 10452 | ACGGGACTCCAGGCAGATGTT |
| 4788 | ACGGGCUUUCCCUGGCCUGGG | 10453 | ACGGGCTTTCCCTGGCCTGGG |
| 4789 | ACGGGGACAUCAUUCUCCCAA | 10454 | ACGGGGACATCATTCTCCCAA |
| 4790 | ACGGGGUUGUCAAAGUUGAUG | 10455 | ACGGGGTTGTCAAAGTTGATG |
| 4791 | ACGGGGUUUCACGUGUUAGCC | 10456 | ACGGGGTTTCACGTGTTAGCC |
| 4792 | ACGGUCCAGCGCAAUUUCCAG | 10457 | ACGGTCCAGCGCAATTTCCAG |
| 4793 | ACGGUGGAGAGAAACUCAAAA | 10458 | ACGGTGGAGAGAAACTCAAAA |
| 4794 | ACGGUGGAUGUCUCCUGGGUG | 10459 | ACGGTGGATGTCTCCTGGGTG |
| 4795 | ACGGUGUCAUAGGAAGAGACG | 10460 | ACGGTGTCATAGGAAGAGACG |
| 4796 | ACGGUUCCCUGCACUGGGGGG | 10461 | ACGGTTCCCTGCACTGGGGGG |
| 4797 | ACGUAAAGACCCCUACAGCGC | 10462 | ACGTAAAGACCCCTACAGCGC |
| 4798 | ACGUCAUCCUCCAGACUGACC | 10463 | ACGTCATCCTCCAGACTGACC |
| 4799 | ACGUCGCCCAGAGCUUGGUGA | 10464 | ACGTCGCCCAGAGCTTGGTGA |
| 4800 | ACGUCUCCUGGGACUCAUCAG | 10465 | ACGTCTCCTGGGACTCATCAG |
| 4801 | ACGUCUUGGGGGAGCACGUCU | 10466 | ACGTCTTGGGGGAGCACGTCT |
| 4802 | ACGUCUUGGGGGGACAGCCUU | 10467 | ACGTCTTGGGGGGACAGCCTT |
| 4803 | ACGUGCCACCGUACCCAGCUG | 10468 | ACGTGCCACCGTACCCAGCTG |
| 4804 | ACGUGCUCCGAAACCAGAAAG | 10469 | ACGTGCTCCGAAACCAGAAAG |
| 4805 | ACGUGGCUGAAGAUCUCGGCU | 10470 | ACGTGGCTGAAGATCTCGGCT |
| 4806 | ACGUGGGAACAGCCGCCGUUG | 10471 | ACGTGGGAACAGCCGCCGTTG |
| 4807 | ACGUGUCCUCACGGCCAAGGU | 10472 | ACGTGTCCTCACGGCCAAGGT |
| 4808 | ACGUGUUAGCCAGGAUGGUCU | 10473 | ACGTGTTAGCCAGGATGGTCT |
| 4809 | ACGUUCCUCAGGUUGGGGAUG | 10474 | ACGTTCCTCAGGTTGGGGATG |
| 4810 | ACGUUGACUCCUGAGUUUAGG | 10475 | ACGTTGACTCCTGAGTTTAGG |
| 4811 | ACGUUUCAACCUCGUAAGGAA | 10476 | ACGTTTCAACCTCGTAAGGAA |
| 4812 | ACGUUUUCCUCUUCACGCCCU | 10477 | ACGTTTTCCTCTTCACGCCCT |
| 4813 | ACUACAGGUGCCCGCCACCAC | 10478 | ACTACAGGTGCCCGCCACCAC |
| 4814 | ACUAGGUGGGCGGACAUAAAG | 10479 | ACTAGGTGGGCGGACATAAAG |
| 4815 | ACUAUCUCCACCGUGGUGAGC | 10480 | ACTATCTCCACCGTGGTGAGC |
| 4816 | ACUAUCUGUACAGGGACGCAU | 10481 | ACTATCTGTACAGGGACGCAT |
| 4817 | ACUAUGUUGCCCAGGCUGGAA | 10482 | ACTATGTTGCCCAGGCTGGAA |
| 4818 | ACUCAAAACUUCCUGGAGAGA | 10483 | ACTCAAAACTTCCTGGAGAGA |
| 4819 | ACUCAAGUGAUCCAGCUCAGC | 10484 | ACTCAAGTGATCCAGCTCAGC |
| 4820 | ACUCAGCGCUGCCAUCGCAGA | 10485 | ACTCAGCGCTGCCATCGCAGA |
| 4821 | ACUCAGGCUCUGCCUCUGGGA | 10486 | ACTCAGGCTCTGCCTCTGGGA |
| 4822 | ACUCAUACAUACAACGGGGAC | 10487 | ACTCATACATACAACGGGGAC |
| 4823 | ACUCAUCAGAGCCAUCCUGGC | 10488 | ACTCATCAGAGCCATCCTGGC |
| 4824 | ACUCAUCCGAGCCAUCUUCGC | 10489 | ACTCATCCGAGCCATCTTCGC |
| 4825 | ACUCAUCGAUAUCGCACUCUU | 10490 | ACTCATCGATATCGCACTCTT |
| 4826 | ACUCAUCGAUAUCUUCGCAUC | 10491 | ACTCATCGATATCTTCGCATC |
| 4827 | ACUCCAGGCAGAUGUUCACGC | 10492 | ACTCCAGGCAGATGTTCACGC |
| 4828 | ACUCCUCUUGGCUGGGUGAGG | 10493 | ACTCCTCTTGGCTGGGTGAGG |
| 4829 | ACUCCUGAACUCAAGUGAUCC | 10494 | ACTCCTGAACTCAAGTGATCC |
| 4830 | ACUCCUGAGUUUAGGGGUUUG | 10495 | ACTCCTGAGTTTAGGGGTTTG |
| 4831 | ACUCGAAGGCCGAGCAGGGGC | 10496 | ACTCGAAGGCCGAGCAGGGGC |
| 4832 | ACUCGCCACUUAGGCAGUGGA | 10497 | ACTCGCCACTTAGGCAGTGGA |
| 4833 | ACUCGCUCCGGUCCAGCGUCA | 10498 | ACTCGCTCCGGTCCAGCGTCA |
| 4834 | ACUCGUAGCCGAUCUUAAGGU | 10499 | ACTCGTAGCCGATCTTAAGGT |
| 4835 | ACUCGUCCUGGGAGCACGUCU | 10500 | ACTCGTCCTGGGAGCACGTCT |
| 4836 | ACUCGUGUAUGUCGCGGAGGC | 10501 | ACTCGTGTATGTCGCGGAGGC |
| 4837 | ACUCGUUUCUUUCGCAUCUGU | 10502 | ACTCGTTTCTTTCGCATCTGT |
| 4838 | ACUCUGAACUGAGAAAGUGCA | 10503 | ACTCTGAACTGAGAAAGTGCA |
| 4839 | ACUCUUUGAUGGGUUCAUCUG | 10504 | ACTCTTTGATGGGTTCATCTG |
| 4840 | ACUGAAAAUGGCUUCGUUGAU | 10505 | ACTGAAAATGGCTTCGTTGAT |
| 4841 | ACUGAACAAAUACAGCAACCA | 10506 | ACTGAACAAATACAGCAACCA |
| 4842 | ACUGAAUGUUUUCAGUCACCA | 10507 | ACTGAATGTTTTCAGTCACCA |
| 4843 | ACUGACCAUCGAGGGGUAGCU | 10508 | ACTGACCATCGAGGGGTAGCT |
| 4844 | ACUGACCAUCUGUCUCGAGGG | 10509 | ACTGACCATCTGTCTCGAGGG |
| 4845 | ACUGAGAAAGUGCAAGGAGAC | 10510 | ACTGAGAAAGTGCAAGGAGAC |
| 4846 | ACUGAGGAAUGCAGCGGUUGA | 10511 | ACTGAGGAATGCAGCGGTTGA |
| 4847 | ACUGAGGAGAUCUAGGGUGAU | 10512 | ACTGAGGAGATCTAGGGTGAT |
| 4848 | ACUGCAACCUCCACCUCCUGG | 10513 | ACTGCAACCTCCACCTCCTGG |
| 4849 | ACUGCAAGCUCCGCUUCCCGG | 10514 | ACTGCAAGCTCCGCTTCCCGG |
| 4850 | ACUGCAGCCUCAGCCUCUCUU | 10515 | ACTGCAGCCTCAGCCTCTCTT |
| 4851 | ACUGCAGCCUCAGCCUCUGUG | 10516 | ACTGCAGCCTCAGCCTCTGTG |
| 4852 | ACUGCAGUCCCCGCCGCGGCG | 10517 | ACTGCAGTCCCCGCCGCGGCG |
| 4853 | ACUGCAGUGGCGCAAUCUCGG | 10518 | ACTGCAGTGGCGCAATCTCGG |
| 4854 | ACUGCCGAGAGAUGCACUUCC | 10519 | ACTGCCGAGAGATGCACTTCC |
| 4855 | ACUGCCGGCUGCCAUGGAUGC | 10520 | ACTGCCGGCTGCCATGGATGC |
| 4856 | ACUGCGCCCGGCCUCACCGUG | 10521 | ACTGCGCCCGGCCTCACCGTG |
| 4857 | ACUGCGGACUGCAGUGGCGCA | 10522 | ACTGCGGACTGCAGTGGCGCA |
| 4858 | ACUGGAACUCGUUUCUUUCGC | 10523 | ACTGGAACTCGTTTCTTTCGC |
| 4859 | ACUGGAAGCUGGCGGGACCAC | 10524 | ACTGGAAGCTGGCGGGACCAC |
| 4860 | ACUGGAAUUCGUCAGGGCGAC | 10525 | ACTGGAATTCGTCAGGGCGAC |
| 4861 | ACUGGAGGAUUUAUUUAUUUU | 10526 | ACTGGAGGATTTATTTATTTT |
| 4862 | ACUGGCACUUGUAGCCACCCU | 10527 | ACTGGCACTTGTAGCCACCCT |
| 4863 | ACUGGCAGCCGCCAUUGCUCA | 10528 | ACTGGCAGCCGCCATTGCTCA |
| 4864 | ACUGGGCUUCUUCUCAUUUCC | 10529 | ACTGGGCTTCTTCTCATTTCC |
| 4865 | ACUGGGGGGGACACAGUGUUU | 10530 | ACTGGGGGGGACACAGTGTTT |
| 4866 | ACUGGGUGGGUCAUGAUUCGG | 10531 | ACTGGGTGGGTCATGATTCGG |
| 4867 | ACUGUAGGGAGAGGCACGCUC | 10532 | ACTGTAGGGAGAGGCACGCTC |
| 4868 | ACUGUCCCCUUGGAACACGUA | 10533 | ACTGTCCCCTTGGAACACGTA |
| 4869 | ACUGUCCGAAGCCUGUUCUGC | 10534 | ACTGTCCGAAGCCTGTTCTGC |
| 4870 | ACUGUCUCUCCGGACAUCAGU | 10535 | ACTGTCTCTCCGGACATCAGT |
| 4871 | ACUUAAUAAAUAUUAAGGGUG | 10536 | ACTTAATAAATATTAAGGGTG |
| 4872 | ACUUAGGCAGUGGAACUCGAA | 10537 | ACTTAGGCAGTGGAACTCGAA |
| 4873 | ACUUCAUCGCUCAUGUCCUUG | 10538 | ACTTCATCGCTCATGTCCTTG |
| 4874 | ACUUCCCAUCGUGGCAGCGAA | 10539 | ACTTCCCATCGTGGCAGCGAA |
| 4875 | ACUUCCUGGAGAGAAAUGGAG | 10540 | ACTTCCTGGAGAGAAATGGAG |
| 4876 | ACUUCGCCAGGAGGGCCCAGG | 10541 | ACTTCGCCAGGAGGGCCCAGG |
| 4877 | ACUUGAACUUGUUGGGUCCCU | 10542 | ACTTGAACTTGTTGGGTCCCT |
| 4878 | ACUUGAUCGGGGCUGGUGGGG | 10543 | ACTTGATCGGGGCTGGTGGGG |
| 4879 | ACUUGCUUCUCUGGGCCUGAC | 10544 | ACTTGCTTCTCTGGGCCTGAC |
| 4880 | ACUUGGCCAUCGCACCUCCAG | 10545 | ACTTGGCCATCGCACCTCCAG |
| 4881 | ACUUGGGCGAGUGGGGGUUGA | 10546 | ACTTGGGCGAGTGGGGGTTGA |
| 4882 | ACUUGUAGCCACCCUCCAGGU | 10547 | ACTTGTAGCCACCCTCCAGGT |
| 4883 | ACUUGUAGGAGAUGCAUUUCC | 10548 | ACTTGTAGGAGATGCATTTCC |
| 4884 | ACUUGUCGUCGCCUUUGUGAC | 10549 | ACTTGTCGTCGCCTTTGTGAC |
| 4885 | ACUUGUUGGGUCCCUCGCAGA | 10550 | ACTTGTTGGGTCCCTCGCAGA |
| 4886 | ACUUUGUCCAGGGUGAUGCAU | 10551 | ACTTTGTCCAGGGTGATGCAT |
| 4887 | ACUUUGUCCUCAAAGACGGCC | 10552 | ACTTTGTCCTCAAAGACGGCC |
| 4888 | ACUUUUUGGUUUUUGUAGAGA | 10553 | ACTTTTTGGTTTTTGTAGAGA |
| 4889 | AGAAAACAGUGCAAAAUAAUA | 10554 | AGAAAACAGTGCAAAATAATA |
| 4890 | AGAAACCAAAAUCCCAACCCA | 10555 | AGAAACCAAAATCCCAACCCA |
| 4891 | AGAAACUCAAAACUUCCUGGA | 10556 | AGAAACTCAAAACTTCCTGGA |
| 4892 | AGAAAGAACCUGAAGUCCCGU | 10557 | AGAAAGAACCTGAAGTCCCGT |
| 4893 | AGAAAGGCUUAUCACGGUUCC | 10558 | AGAAAGGCTTATCACGGTTCC |
| 4894 | AGAAAGUGCAAGGAGACCACG | 10559 | AGAAAGTGCAAGGAGACCACG |
| 4895 | AGAAAUGGAGGUGUCAUCCUG | 10560 | AGAAATGGAGGTGTCATCCTG |
| 4896 | AGAAAUUUAAUUGAUCACAGU | 10561 | AGAAATTTAATTGATCACAGT |
| 4897 | AGAACCAUAUCCUCUGGGGAC | 10562 | AGAACCATATCCTCTGGGGAC |
| 4898 | AGAACCUGAAGUCCCGUCAAA | 10563 | AGAACCTGAAGTCCCGTCAAA |
| 4899 | AGAACUGAGGAAUGCAGCGGU | 10564 | AGAACTGAGGAATGCAGCGGT |
| 4900 | AGAAGAGGCAUUUCAGAGGCA | 10565 | AGAAGAGGCATTTCAGAGGCA |
| 4901 | AGAAGAGGUAAACACGUUGAC | 10566 | AGAAGAGGTAAACACGTTGAC |
| 4902 | AGAAGAGGUAGGCGAUGGAGC | 10567 | AGAAGAGGTAGGCGATGGAGC |
| 4903 | AGAAGCCACUCAUACAUACAA | 10568 | AGAAGCCACTCATACATACAA |
| 4904 | AGAAGGAAGACCCCCAGGCAA | 10569 | AGAAGGAAGACCCCCAGGCAA |
| 4905 | AGAAGGGGUGGGCCAGCCUCU | 10570 | AGAAGGGGTGGGCCAGCCTCT |
| 4906 | AGAAGUGGAAGCACUAGGUGG | 10571 | AGAAGTGGAAGCACTAGGTGG |
| 4907 | AGAAUAGUCACUGAACAAAUA | 10572 | AGAATAGTCACTGAACAAATA |
| 4908 | AGACAAGCACGUCUCCUGGGA | 10573 | AGACAAGCACGTCTCCTGGGA |
| 4909 | AGACACUGGGUGGGUCAUGAU | 10574 | AGACACTGGGTGGGTCATGAT |
| 4910 | AGACAGCCUUGCUCGUCUGAG | 10575 | AGACAGCCTTGCTCGTCTGAG |
| 4911 | AGACAGUGCCCAGGACAGAGU | 10576 | AGACAGTGCCCAGGACAGAGT |
| 4912 | AGACAUUCAGGCUACAAAAAC | 10577 | AGACATTCAGGCTACAAAAAC |
| 4913 | AGACAUUGUCACUAUCUCCAC | 10578 | AGACATTGTCACTATCTCCAC |
| 4914 | AGACCAAGGAGGGGGGGGGAG | 10579 | AGACCAAGGAGGGGCGGGGAG |
| 4915 | AGACCACGGGAAUGGAAGUGG | 10580 | AGACCACGGGAATGGAAGTGG |
| 4916 | AGACCAGAGACCCAGCUCCGU | 10581 | AGACCAGAGACCCAGCTCCGT |
| 4917 | AGACCAGUAGAUUCUAUUGCU | 10582 | AGACCAGTAGATTCTATTGCT |
| 4918 | AGACCCACUUGUAGGAGAUGC | 10583 | AGACCCACTTGTAGGAGATGC |
| 4919 | AGACCCAGCUCCGUGGCGAUG | 10584 | AGACCCAGCTCCGTGGCGATG |
| 4920 | AGACCCCCAGGCAAAGGAAGA | 10585 | AGACCCCCAGGCAAAGGAAGA |
| 4921 | AGACCCCUACAGCGCUGCGGC | 10586 | AGACCCCTACAGCGCTGCGGC |
| 4922 | AGACGAACUGCCGAGAGAUGC | 10587 | AGACGAACTGCCGAGAGATGC |
| 4923 | AGACGACAAUGGCAGUUCUCG | 10588 | AGACGACAATGGCAGTTCTCG |
| 4924 | AGACGAGGAGCACGAUGGGGA | 10589 | AGACGAGGAGCACGATGGGGA |
| 4925 | AGACGAGGAGCACUGUAGGGA | 10590 | AGACGAGGAGCACTGTAGGGA |
| 4926 | AGACGCCGUGGGCUCUGUCAA | 10591 | AGACGCCGTGGGCTCTGTCAA |
| 4927 | AGACGGAGUCUCGCUCUGUCG | 10592 | AGACGGAGTCTCGCTCTGTCG |
| 4928 | AGACGGCCAAGGAGAAGGGGU | 10593 | AGACGGCCAAGGAGAAGGGGT |
| 4929 | AGACGGGGUUGUCAAAGUUGA | 10594 | AGACGGGGTTGTCAAAGTTGA |
| 4930 | AGACGGGGUUUCACGUGUUAG | 10595 | AGACGGGGTTTCACGTGTTAG |
| 4931 | AGACGUGGGAACAGCCGCCGU | 10596 | AGACGTGGGAACAGCCGCCGT |
| 4932 | AGACUGACCAUCGAGGGGUAG | 10597 | AGACTGACCATCGAGGGGTAG |
| 4933 | AGACUGACCAUCUGUCUCGAG | 10598 | AGACTGACCATCTGTCTCGAG |
| 4934 | AGACUGCGGACUGCAGUGGCG | 10599 | AGACTGCGGACTGCAGTGGCG |
| 4935 | AGACUGGAGGAUUUAUUUAUU | 10600 | AGACTGGAGGATTTATTTATT |
| 4936 | AGACUUUGUCCAGGGUGAUGC | 10601 | AGACTTTGTCCAGGGTGATGC |
| 4937 | AGAGAAACUCAAAACUUCCUG | 10602 | AGAGAAACTCAAAACTTCCTG |
| 4938 | AGAGAAAUGGAGGUGUCAUCC | 10603 | AGAGAAATGGAGGTGTCATCC |
| 4939 | AGAGAACCAUAUCCUCUGGGG | 10604 | AGAGAACCATATCCTCTGGGG |
| 4940 | AGAGACAGUGCCCAGGACAGA | 10605 | AGAGACAGTGCCCAGGACAGA |
| 4941 | AGAGACCCAGCUCCGUGGCGA | 10606 | AGAGACCCAGCTCCGTGGCGA |
| 4942 | AGAGACGCCGUGGGCUCUGUC | 10607 | AGAGACGCCGTGGGCTCTGTC |
| 4943 | AGAGACGGGGUUUCACGUGUU | 10608 | AGAGACGGGGTTTCACGTGTT |
| 4944 | AGAGAUGCACUUCCCAUCGUG | 10609 | AGAGATGCACTTCCCATCGTG |
| 4945 | AGAGAUGGGAUCUCGCUUUGU | 10610 | AGAGATGGGATCTCGCTTTGT |
| 4946 | AGAGAUGGGGUCUUACUAUGU | 10611 | AGAGATGGGGTCTTACTATGT |
| 4947 | AGAGAUUGGUGGAUGAGGCAG | 10612 | AGAGATTGGTGGATGAGGCAG |
| 4948 | AGAGCACAGCGGAAACCGGGG | 10613 | AGAGCACAGCGGAAACCGGGG |
| 4949 | AGAGCACUGGAAUUCGUCAGG | 10614 | AGAGCACTGGAATTCGTCAGG |
| 4950 | AGAGCCAUCCUGGCACUCAGC | 10615 | AGAGCCATCCTGGCACTCAGC |
| 4951 | AGAGCCCUCACGCUACUGGGC | 10616 | AGAGCCCTCACGCTACTGGGC |
| 4952 | AGAGCGACCACGUUCCUCAGG | 10617 | AGAGCGACCACGTTCCTCAGG |
| 4953 | AGAGCUGGCUGCAGGUGUCGG | 10618 | AGAGCTGGCTGCAGGTGTCGG |
| 4954 | AGAGCUUGGUGAGACAUUGUC | 10619 | AGAGCTTGGTGAGACATTGTC |
| 4955 | AGAGGCAAUAACCCCCUACAC | 10620 | AGAGGCAATAACCCCCTACAC |
| 4956 | AGAGGCACGCUCUGUCACACA | 10621 | AGAGGCACGCTCTGTCACACA |
| 4957 | AGAGGCAUUUCAGAGGCAAUA | 10622 | AGAGGCATTTCAGAGGCAATA |
| 4958 | AGAGGCCAUGGGCUGCUGAUG | 10623 | AGAGGCCATGGGCTGCTGATG |
| 4959 | AGAGGCGGCCACUGAGGAGAU | 10624 | AGAGGCGGCCACTGAGGAGAT |
| 4960 | AGAGGUAAACACGUUGACUCC | 10625 | AGAGGTAAACACGTTGACTCC |
| 4961 | AGAGGUAGGCGAUGGAGCCCA | 10626 | AGAGGTAGGCGATGGAGCCCA |
| 4962 | AGAGUCGGUCCAGUAGAUGUU | 10627 | AGAGTCGGTCCAGTAGATGTT |
| 4963 | AGAGUGUCACAUUAACGCAGC | 10628 | AGAGTGTCACATTAACGCAGC |
| 4964 | AGAGUGUCAGACAGCCUUGCU | 10629 | AGAGTGTCAGACAGCCTTGCT |
| 4965 | AGAUACUGGCAGCCGCCAUUG | 10630 | AGATACTGGCAGCCGCCATTG |
| 4966 | AGAUCAUUCUCUGGGACAGGU | 10631 | AGATCATTCTCTGGGACAGGT |
| 4967 | AGAUCAUUUCCGACGCCAUGA | 10632 | AGATCATTTCCGACGCCATGA |
| 4968 | AGAUCUAGGGUGAUGCCAUUG | 10633 | AGATCTAGGGTGATGCCATTG |
| 4969 | AGAUCUCGGCUGCAGACCAAG | 10634 | AGATCTCGGCTGCAGACCAAG |
| 4970 | AGAUGAAUAAAUAUAUAAAAC | 10635 | AGATGAATAAATATATAAAAC |
| 4971 | AGAUGCACUUCCCAUCGUGGC | 10636 | AGATGCACTTCCCATCGTGGC |
| 4972 | AGAUGCAUUUCCCGUCUUGGC | 10637 | AGATGCATTTCCCGTCTTGGC |
| 4973 | AGAUGGAGUGAAGUUUGGAGU | 10638 | AGATGGAGTGAAGTTTGGAGT |
| 4974 | AGAUGGGAUCUCGCUUUGUUG | 10639 | AGATGGGATCTCGCTTTGTTG |
| 4975 | AGAUGGGGUCUUACUAUGUUG | 10640 | AGATGGGGTCTTACTATGTTG |
| 4976 | AGAUGGUCUUCCGGUUGCCCC | 10641 | AGATGGTCTTCCGGTTGCCCC |
| 4977 | AGAUGUCCACACCAUUCAGGC | 10642 | AGATGTCCACACCATTCAGGC |
| 4978 | AGAUGUUCACGCCACGUCAUC | 10643 | AGATGTTCACGCCACGTCATC |
| 4979 | AGAUGUUGCUGUGGAUCCAGU | 10644 | AGATGTTGCTGTGGATCCAGT |
| 4980 | AGAUUCUAUUGCUGGCCACCU | 10645 | AGATTCTATTGCTGGCCACCT |
| 4981 | AGAUUCUGAGGCAACGUUUCA | 10646 | AGATTCTGAGGCAACGTTTCA |
| 4982 | AGAUUGGGGAAGUGAAUGGCU | 10647 | AGATTGGGGAAGTGAATGGCT |
| 4983 | AGAUUGGUGGAUGAGGCAGGG | 10648 | AGATTGGTGGATGAGGCAGGG |
| 4984 | AGAUUUGUCCUUGCAGUCGGG | 10649 | AGATTTGTCCTTGCAGTCGGG |
| 4985 | AGAUUUUAUAUAUAUGUGUGU | 10650 | AGATTTTATATATATGTGTGT |
| 4986 | AGCAACCAGGGUUUGCAAAAA | 10651 | AGCAACCAGGGTTTGCAAAAA |
| 4987 | AGCAACGUCGCCCAGAGCUUG | 10652 | AGCAACGTCGCCCAGAGCTTG |
| 4988 | AGCAAGGCGACGGUCCAGCGC | 10653 | AGCAAGGCGACGGTCCAGCGC |
| 4989 | AGCAAGGCUUGCAUAGAAGAG | 10654 | AGCAAGGCTTGCATAGAAGAG |
| 4990 | AGCACAGCGGAAACCGGGGAC | 10655 | AGCACAGCGGAAACCGGGGAC |
| 4991 | AGCACCGGGCAGGAGGCCUCG | 10656 | AGCACCGGGCAGGAGGCCTCG |
| 4992 | AGCACGAUGGGGAGGACAAUG | 10657 | AGCACGATGGGGAGGACAATG |
| 4993 | AGCACGUCUCCUGGGACUCAU | 10658 | AGCACGTCTCCTGGGACTCAT |
| 4994 | AGCACGUCUUGGGGGAGCACG | 10659 | AGCACGTCTTGGGGGAGCACG |
| 4995 | AGCACGUCUUGGGGGGACAGC | 10660 | AGCACGTCTTGGGGGGACAGC |
| 4996 | AGCACUAGGUGGGCGGACAUA | 10661 | AGCACTAGGTGGGCGGACATA |
| 4997 | AGCACUGGAAUUCGUCAGGGC | 10662 | AGCACTGGAATTCGTCAGGGC |
| 4998 | AGCACUGUAGGGAGAGGCACG | 10663 | AGCACTGTAGGGAGAGGCACG |
| 4999 | AGCAGCAUGCCGUCCGGGCAG | 10664 | AGCAGCATGCCGTCCGGGCAG |
| 5000 | AGCAGGGGCUACUGUCCCCUU | 10665 | AGCAGGGGCTACTGTCCCCTT |
| 5001 | AGCAGUCCCGGUCUGAGUCAC | 10666 | AGCAGTCCCGGTCTGAGTCAC |
| 5002 | AGCAGUGGCAUAAUCAUAGCU | 10667 | AGCAGTGGCATAATCATAGCT |
| 5003 | AGCAUGCCGUCCGGGCAGGCG | 10668 | AGCATGCCGTCCGGGCAGGCG |
| 5004 | AGCAUUCGUUGGUCCCGCACU | 10669 | AGCATTCGTTGGTCCCGCACT |
| 5005 | AGCCAACAAGUUGACAUCGGA | 10670 | AGCCAACAAGTTGACATCGGA |
| 5006 | AGCCAACUUCAUCGCUCAUGU | 10671 | AGCCAACTTCATCGCTCATGT |
| 5007 | AGCCACACCUGGGUGCAGGCC | 10672 | AGCCACACCTGGGTGCAGGCC |
| 5008 | AGCCACCCUCCAGGUUCACGC | 10673 | AGCCACCCTCCAGGTTCACGC |
| 5009 | AGCCACUCAUACAUACAACGG | 10674 | AGCCACTCATACATACAACGG |
| 5010 | AGCCACUGCGCCCGGCCUCAC | 10675 | AGCCACTGCGCCCGGCCTCAC |
| 5011 | AGCCACUUGCUUCUCUGGGCC | 10676 | AGCCACTTGCTTCTCTGGGCC |
| 5012 | AGCCAGAUCAUUUCCGACGCC | 10677 | AGCCAGATCATTTCCGACGCC |
| 5013 | AGCCAGCCCGUCGGGGGCCUG | 10678 | AGCCAGCCCGTCGGGGGCCTG |
| 5014 | AGCCAGGAUGGUCUCGAUCUC | 10679 | AGCCAGGATGGTCTCGATCTC |
| 5015 | AGCCAUCCUGGCACUCAGCGC | 10680 | AGCCATCCTGGCACTCAGCGC |
| 5016 | AGCCAUCUUCGCAGUCGGGGU | 10681 | AGCCATCTTCGCAGTCGGGGT |
| 5017 | AGCCAUGAACAGGAUCCACCA | 10682 | AGCCATGAACAGGATCCACCA |
| 5018 | AGCCAUGUUGCAGACUUUGUC | 10683 | AGCCATGTTGCAGACTTTGTC |
| 5019 | AGCCAUUGCAUGGGCACUGUC | 10684 | AGCCATTGCATGGGCACTGTC |
| 5020 | AGCCCACAGCCUUGCAGGCCU | 10685 | AGCCCACAGCCTTGCAGGCCT |
| 5021 | AGCCCAGGGGUGGCCCCAGGC | 10686 | AGCCCAGGGGTGGCCCCAGGC |
| 5022 | AGCCCCAGGGCCCCAUGCUCG | 10687 | AGCCCCAGGGCCCCATGCTCG |
| 5023 | AGCCCGUCGGGGGCCUGGAUG | 10688 | AGCCCGTCGGGGGCCTGGATG |
| 5024 | AGCCCUCACGCUACUGGGCUU | 10689 | AGCCCTCACGCTACTGGGCTT |
| 5025 | AGCCGAUCUUAAGGUCAUUGC | 10690 | AGCCGATCTTAAGGTCATTGC |
| 5026 | AGCCGCCAGUUCUUCCAUAGA | 10691 | AGCCGCCAGTTCTTCCATAGA |
| 5027 | AGCCGCCAUUGCUCAGGGUGG | 10692 | AGCCGCCATTGCTCAGGGTGG |
| 5028 | AGCCGCCGUUGUUGUCCAAGC | 10693 | AGCCGCCGTTGTTGTCCAAGC |
| 5029 | AGCCGGGAGGUGUCGGGAACA | 10694 | AGCCGGGAGGTGTCGGGAACA |
| 5030 | AGCCGUCCAAGCAGUCCCGGU | 10695 | AGCCGTCCAAGCAGTCCCGGT |
| 5031 | AGCCGUCCUGGUUGUGGCAAA | 10696 | AGCCGTCCTGGTTGTGGCAAA |
| 5032 | AGCCGUCGGGGCACAGGCACU | 10697 | AGCCGTCGGGGCACAGGCACT |
| 5033 | AGCCGUUCUCCCUGAAUAACG | 10698 | AGCCGTTCTCCCTGAATAACG |
| 5034 | AGCCGUUGUCGCAGUCCACUU | 10699 | AGCCGTTGTCGCAGTCCACTT |
| 5035 | AGCCUCAAAUGCCUGAGCUCA | 10700 | AGCCTCAAATGCCTGAGCTCA |
| 5036 | AGCCUCAGCCUCUCUUGGCUG | 10701 | AGCCTCAGCCTCTCTTGGCTG |
| 5037 | AGCCUCAGCCUCUGUGAGGCA | 10702 | AGCCTCAGCCTCTGTGAGGCA |
| 5038 | AGCCUCCCAAAGUGCUGGGAU | 10703 | AGCCTCCCAAAGTGCTGGGAT |
| 5039 | AGCCUCCCAAGUAGCUGGGAU | 10704 | AGCCTCCCAAGTAGCTGGGAT |
| 5040 | AGCCUCCCGAGUAGCUGGGAC | 10705 | AGCCTCCCGAGTAGCTGGGAC |
| 5041 | AGCCUCGCCAUGCCUGGCCAG | 10706 | AGCCTCGCCATGCCTGGCCAG |
| 5042 | AGCCUCUCUUGGCUGGGUGAG | 10707 | AGCCTCTCTTGGCTGGGTGAG |
| 5043 | AGCCUCUGCCAGGCAGUGUCC | 10708 | AGCCTCTGCCAGGCAGTGTCC |
| 5044 | AGCCUCUGUGAGGCAGCUCCU | 10709 | AGCCTCTGTGAGGCAGCTCCT |
| 5045 | AGCCUCUUUUCAUCCUCCAAG | 10710 | AGCCTCTTTTCATCCTCCAAG |
| 5046 | AGCCUGACGGUGGAUGUCUCC | 10711 | AGCCTGACGGTGGATGTCTCC |
| 5047 | AGCCUGUUCUGCCUCCCAGAU | 10712 | AGCCTGTTCTGCCTCCCAGAT |
| 5048 | AGCCUUCCUCACACUGGCACU | 10713 | AGCCTTCCTCACACTGGCACT |
| 5049 | AGCCUUGCAGGCCUUCGUGUG | 10714 | AGCCTTGCAGGCCTTCGTGTG |
| 5050 | AGCCUUGCUCGUCUGAGCCGU | 10715 | AGCCTTGCTCGTCTGAGCCGT |
| 5051 | AGCGAAACUCGUCCUGGGAGC | 10716 | AGCGAAACTCGTCCTGGGAGC |
| 5052 | AGCGACCACGUUCCUCAGGUU | 10717 | AGCGACCACGTTCCTCAGGTT |
| 5053 | AGCGAGUAGAUGUCCACACCA | 10718 | AGCGAGTAGATGTCCACACCA |
| 5054 | AGCGCAAUUUCCAGCCCCAGG | 10719 | AGCGCAATTTCCAGCCCCAGG |
| 5055 | AGCGCAGUUUUCCUCGUCAGA | 10720 | AGCGCAGTTTTCCTCGTCAGA |
| 5056 | AGCGCCAGCUGGAGUGGAUGC | 10721 | AGCGCCAGCTGGAGTGGATGC |
| 5057 | AGCGCUGCCAUCGCAGACCCA | 10722 | AGCGCTGCCATCGCAGACCCA |
| 5058 | AGCGCUGCGGCCACUCAUCCG | 10723 | AGCGCTGCGGCCACTCATCCG |
| 5059 | AGCGGAAACCGGGGACUUCGC | 10724 | AGCGGAAACCGGGGACTTCGC |
| 5060 | AGCGGUUGACACGGCCCCCAC | 10725 | AGCGGTTGACACGGCCCCCAC |
| 5061 | AGCGUCAUCUUCCUGACCUCG | 10726 | AGCGTCATCTTCCTGACCTCG |
| 5062 | AGCGUUGCACACAGUCCAGUU | 10727 | AGCGTTGCACACAGTCCAGTT |
| 5063 | AGCUCAAACCAUCCUCCCACC | 10728 | AGCTCAAACCATCCTCCCACC |
| 5064 | AGCUCACGACAGCCUCAAAUG | 10729 | AGCTCACGACAGCCTCAAATG |
| 5065 | AGCUCAGCCUCCCAAAGUGCU | 10730 | AGCTCAGCCTCCCAAAGTGCT |
| 5066 | AGCUCCGCUUCCCGGGUUCAC | 10731 | AGCTCCGCTTCCCGGGTTCAC |
| 5067 | AGCUCCGUGGCGAUGGGGAGG | 10732 | AGCTCCGTGGCGATGGGGAGG |
| 5068 | AGCUCCUCAUGUCCCUGGCCA | 10733 | AGCTCCTCATGTCCCTGGCCA |
| 5069 | AGCUCUGGCAGGCAAUGCUUU | 10734 | AGCTCTGGCAGGCAATGCTTT |
| 5070 | AGCUGAAGUCCCCGGAUUUGC | 10735 | AGCTGAAGTCCCCGGATTTGC |
| 5071 | AGCUGACCUUUAGCCUGACGG | 10736 | AGCTGACCTTTAGCCTGACGG |
| 5072 | AGCUGAUUUUUAACUUUUUGG | 10737 | AGCTGATTTTTAACTTTTTGG |
| 5073 | AGCUGCCCCCGAGACCAGAGA | 10738 | AGCTGCCCCCGAGACCAGAGA |
| 5074 | AGCUGGAAACCCUGGCUUCCC | 10739 | AGCTGGAAACCCTGGCTTCCC |
| 5075 | AGCUGGAAGCCGUCGGGGCAC | 10740 | AGCTGGAAGCCGTCGGGGCAC |
| 5076 | AGCUGGAAGCCUUCCUCACAC | 10741 | AGCTGGAAGCCTTCCTCACAC |
| 5077 | AGCUGGAGUGGAUGCACUCGC | 10742 | AGCTGGAGTGGATGCACTCGC |
| 5078 | AGCUGGCGGGACCACAGGUGA | 10743 | AGCTGGCGGGACCACAGGTGA |
| 5079 | AGCUGGCUGCAGGUGUCGGGA | 10744 | AGCTGGCTGCAGGTGTCGGGA |
| 5080 | AGCUGGGACUACAGGUGCCCG | 10745 | AGCTGGGACTACAGGTGCCCG |
| 5081 | AGCUGGGAUCACAGGCACGUG | 10746 | AGCTGGGATCACAGGCACGTG |
| 5082 | AGCUGGGGGAUGCAGGUGGAG | 10747 | AGCTGGGGGATGCAGGTGGAG |
| 5083 | AGCUGGGUGCUGCAGAUCAUU | 10748 | AGCTGGGTGCTGCAGATCATT |
| 5084 | AGCUGUAGCCGUCCUGGUUGU | 10749 | AGCTGTAGCCGTCCTGGTTGT |
| 5085 | AGCUGUUGCACUGGAAGCUGG | 10750 | AGCTGTTGCACTGGAAGCTGG |
| 5086 | AGCUUGGUGAGACAUUGUCAC | 10751 | AGCTTGGTGAGACATTGTCAC |
| 5087 | AGGAAAGGAAGAAACCAAAAU | 10752 | AGGAAAGGAAGAAACCAAAAT |
| 5088 | AGGAACCGAGGCCUUGAGGGU | 10753 | AGGAACCGAGGCCTTGAGGGT |
| 5089 | AGGAACCGCGGAGGAACGCCA | 10754 | AGGAACCGCGGAGGAACGCCA |
| 5090 | AGGAAGAAACCAAAAUCCCAA | 10755 | AGGAAGAAACCAAAATCCCAA |
| 5091 | AGGAAGACCCCCAGGCAAAGG | 10756 | AGGAAGACCCCCAGGCAAAGG |
| 5092 | AGGAAGACGAGGAGCACGAUG | 10757 | AGGAAGACGAGGAGCACGATG |
| 5093 | AGGAAGACGAGGAGCACUGUA | 10758 | AGGAAGACGAGGAGCACTGTA |
| 5094 | AGGAAGAGACGCCGUGGGCUC | 10759 | AGGAAGAGACGCCGTGGGCTC |
| 5095 | AGGAAGGGUUCUGGGCAGGGA | 10760 | AGGAAGGGTTCTGGGCAGGGA |
| 5096 | AGGAAGGUACAAACCCACCUC | 10761 | AGGAAGGTACAAACCCACCTC |
| 5097 | AGGAAUGCAGCGGUUGACACG | 10762 | AGGAATGCAGCGGTTGACACG |
| 5098 | AGGACAAGGUGGGGACGGAGG | 10763 | AGGACAAGGTGGGGACGGAGG |
| 5099 | AGGACAAUGGACAGAGCCCUC | 10764 | AGGACAATGGACAGAGCCCTC |
| 5100 | AGGACAGAGUCGGUCCAGUAG | 10765 | AGGACAGAGTCGGTCCAGTAG |
| 5101 | AGGACAGCCUUGCUCGUCUGA | 10766 | AGGACAGCCTTGCTCGTCTGA |
| 5102 | AGGAGAAGGGGUGGGCCAGCC | 10767 | AGGAGAAGGGGTGGGCCAGCC |
| 5103 | AGGAGACCACGGGAAUGGAAG | 10768 | AGGAGACCACGGGAATGGAAG |
| 5104 | AGGAGAUCUAGGGUGAUGCCA | 10769 | AGGAGATCTAGGGTGATGCCA |
| 5105 | AGGAGAUGCAUUUCCCGUCUU | 10770 | AGGAGATGCATTTCCCGTCTT |
| 5106 | AGGAGCAAGGCGACGGUCCAG | 10771 | AGGAGCAAGGCGACGGTCCAG |
| 5107 | AGGAGCACGAUGGGGAGGACA | 10772 | AGGAGCACGATGGGGAGGACA |
| 5108 | AGGAGCACUGUAGGGAGAGGC | 10773 | AGGAGCACTGTAGGGAGAGGC |
| 5109 | AGGAGGCCUCGUCUGAGCCGU | 10774 | AGGAGGCCTCGTCTGAGCCGT |
| 5110 | AGGAGGGCCCAGGAUAACGGA | 10775 | AGGAGGGCCCAGGATAACGGA |
| 5111 | AGGAGGGGGGGGGAGAUUCUG | 10776 | AGGAGGGGGGGGGAGATTCTG |
| 5112 | AGGAUAACGGAGGGCGACUCG | 10777 | AGGATAACGGAGGGCGACTCG |
| 5113 | AGGAUCCACCACGAUGGCCCU | 10778 | AGGATCCACCACGATGGCCCT |
| 5114 | AGGAUGGGCACCAGGCUGGUG | 10779 | AGGATGGGCACCAGGCTGGTG |
| 5115 | AGGAUGGUCUCGAUCUCCUGA | 10780 | AGGATGGTCTCGATCTCCTGA |
| 5116 | AGGAUUGUGUCACGGUGGAGA | 10781 | AGGATTGTGTCACGGTGGAGA |
| 5117 | AGGAUUUAUUUAUUUUUUUAU | 10782 | AGGATTTATTTATTTTTTTAT |
| 5118 | AGGCAAAGGAAGACGAGGAGC | 10783 | AGGCAAAGGAAGACGAGGAGC |
| 5119 | AGGCAACGUUUCAACCUCGUA | 10784 | AGGCAACGTTTCAACCTCGTA |
| 5120 | AGGCAAUAACCCCCUACACAG | 10785 | AGGCAATAACCCCCTACACAG |
| 5121 | AGGCAAUGCUUUGGUCUUCUC | 10786 | AGGCAATGCTTTGGTCTTCTC |
| 5122 | AGGCACAGAUACUGGCAGCCG | 10787 | AGGCACAGATACTGGCAGCCG |
| 5123 | AGGCACCUGUGUCUGGCCAAA | 10788 | AGGCACCTGTGTCTGGCCAAA |
| 5124 | AGGCACGCUCUGUCACACAGC | 10789 | AGGCACGCTCTGTCACACAGC |
| 5125 | AGGCACGUGCCACCGUACCCA | 10790 | AGGCACGTGCCACCGTACCCA |
| 5126 | AGGCACUCGUAGCCGAUCUUA | 10791 | AGGCACTCGTAGCCGATCTTA |
| 5127 | AGGCACUUAAUAAAUAUUAAG | 10792 | AGGCACTTAATAAATATTAAG |
| 5128 | AGGCAGAUGUUCACGCCACGU | 10793 | AGGCAGATGTTCACGCCACGT |
| 5129 | AGGCAGCCGGGAGGUGUCGGG | 10794 | AGGCAGCCGGGAGGTGTCGGG |
| 5130 | AGGCAGCUCCUCAUGUCCCUG | 10795 | AGGCAGCTCCTCATGTCCCTG |
| 5131 | AGGCAGGGCCAGGGGGUCCCG | 10796 | AGGCAGGGCCAGGGGGTCCCG |
| 5132 | AGGCAGUGGAACUCGAAGGCC | 10797 | AGGCAGTGGAACTCGAAGGCC |
| 5133 | AGGCAGUGUCCCGACCCGGAU | 10798 | AGGCAGTGTCCCGACCCGGAT |
| 5134 | AGGCAUUUCAGAGGCAAUAAC | 10799 | AGGCATTTCAGAGGCAATAAC |
| 5135 | AGGCAUUUGCAUAGAAGUGGA | 10800 | AGGCATTTGCATAGAAGTGGA |
| 5136 | AGGCCACGUGUCCUCACGGCC | 10801 | AGGCCACGTGTCCTCACGGCC |
| 5137 | AGGCCAGACUGCGGACUGCAG | 10802 | AGGCCAGACTGCGGACTGCAG |
| 5138 | AGGCCAUGGGCUGCUGAUGCA | 10803 | AGGCCATGGGCTGCTGATGCA |
| 5139 | AGGCCCACAGCUGGGGGAUGC | 10804 | AGGCCCACAGCTGGGGGATGC |
| 5140 | AGGCCCCCUUUCUUGAUCUUG | 10805 | AGGCCCCCTTTCTTGATCTTG |
| 5141 | AGGCCGAGCAGGGGCUACUGU | 10806 | AGGCCGAGCAGGGGCTACTGT |
| 5142 | AGGCCGGCGAGGUCUCAGGAA | 10807 | AGGCCGGCGAGGTCTCAGGAA |
| 5143 | AGGCCUCGUCUGAGCCGUCCA | 10808 | AGGCCTCGTCTGAGCCGTCCA |
| 5144 | AGGCCUGGUCUCCAACUCCUG | 10809 | AGGCCTGGTCTCCAACTCCTG |
| 5145 | AGGCCUUCGUGUGGGGGUCCA | 10810 | AGGCCTTCGTGTGGGGGTCCA |
| 5146 | AGGCCUUGAGGGUGGCCGGGG | 10811 | AGGCCTTGAGGGTGGCCGGGG |
| 5147 | AGGCGACGGUCCAGCGCAAUU | 10812 | AGGCGACGGTCCAGCGCAATT |
| 5148 | AGGCGAUCCUCCUGCCUCAGC | 10813 | AGGCGATCCTCCTGCCTCAGC |
| 5149 | AGGCGAUGGAGCCCACAGCCU | 10814 | AGGCGATGGAGCCCACAGCCT |
| 5150 | AGGCGCAGGUAAACUUGGGCG | 10815 | AGGCGCAGGTAAACTTGGGCG |
| 5151 | AGGCGGCCACUGAGGAGAUCU | 10816 | AGGCGGCCACTGAGGAGATCT |
| 5152 | AGGCGGCUCCGGGGACCCGGG | 10817 | AGGCGGCTCCGGGGACCCGGG |
| 5153 | AGGCGGUUGGCACUGAAAAUG | 10818 | AGGCGGTTGGCACTGAAAATG |
| 5154 | AGGCGUGAGCCACUGCGCCCG | 10819 | AGGCGTGAGCCACTGCGCCCG |
| 5155 | AGGCGUGAGGCACCUGUGUCU | 10820 | AGGCGTGAGGCACCTGTGTCT |
| 5156 | AGGCUAACCUGGCUGUCUAGC | 10821 | AGGCTAACCTGGCTGTCTAGC |
| 5157 | AGGCUACAAAAACCUUCUAUA | 10822 | AGGCTACAAAAACCTTCTATA |
| 5158 | AGGCUCUGCCUCUGGGAAGUG | 10823 | AGGCTCTGCCTCTGGGAAGTG |
| 5159 | AGGCUGGAAAGCAGUGGCAUA | 10824 | AGGCTGGAAAGCAGTGGCATA |
| 5160 | AGGCUGGAGUGCAGUGGCUCG | 10825 | AGGCTGGAGTGCAGTGGCTCG |
| 5161 | AGGCUGGUGUACUCGCUCCGG | 10826 | AGGCTGGTGTACTCGCTCCGG |
| 5162 | AGGCUGGUGUCCUGACAGCCA | 10827 | AGGCTGGTGTCCTGACAGCCA |
| 5163 | AGGCUGUCACUGUCUCUCCGG | 10828 | AGGCTGTCACTGTCTCTCCGG |
| 5164 | AGGCUUAUCACGGUUCCCUGC | 10829 | AGGCTTATCACGGTTCCCTGC |
| 5165 | AGGCUUGCAUAGAAGAGGUAA | 10830 | AGGCTTGCATAGAAGAGGTAA |
| 5166 | AGGGACGCAUUUACGUGCUCC | 10831 | AGGGACGCATTTACGTGCTCC |
| 5167 | AGGGACGGGACUCCAGGCAGA | 10832 | AGGGACGGGACTCCAGGCAGA |
| 5168 | AGGGAGAGGCACGCUCUGUCA | 10833 | AGGGAGAGGCACGCTCTGTCA |
| 5169 | AGGGAGGCACAGAUACUGGCA | 10834 | AGGGAGGCACAGATACTGGCA |
| 5170 | AGGGCAAAGGCUAACCUGGCU | 10835 | AGGGCAAAGGCTAACCTGGCT |
| 5171 | AGGGCCAGGGGGUCCCGUUGG | 10836 | AGGGCCAGGGGGTCCCGTTGG |
| 5172 | AGGGCCCAGGAUAACGGAGGG | 10837 | AGGGCCCAGGATAACGGAGGG |
| 5173 | AGGGCCCCAUGCUCGCAGCCU | 10838 | AGGGCCCCATGCTCGCAGCCT |
| 5174 | AGGGCCCCGAGAAUAGUCACU | 10839 | AGGGCCCCGAGAATAGTCACT |
| 5175 | AGGGCGACAGGUGGCCACAGC | 10840 | AGGGCGACAGGTGGCCACAGC |
| 5176 | AGGGCGACAGGUGGCCACAGG | 10841 | AGGGCGACAGGTGGCCACAGG |
| 5177 | AGGGCGACUCGUGUAUGUCGC | 10842 | AGGGCGACTCGTGTATGTCGC |
| 5178 | AGGGCGCCCCCCAGGACAAGG | 10843 | AGGGCGCCCCCCAGGACAAGG |
| 5179 | AGGGGGGGGAGAUUCUGAGG | 10844 | AGGGGCGGGGAGATTCTGAGG |
| 5180 | AGGGGCUACUGUCCCCUUGGA | 10845 | AGGGGCTACTGTCCCCTTGGA |
| 5181 | AGGGGGUCCCGUUGGUGCCAU | 10846 | AGGGGGTCCCGTTGGTGCCAT |
| 5182 | AGGGGUAGCUGUAGCCGUCCU | 10847 | AGGGGTAGCTGTAGCCGTCCT |
| 5183 | AGGGGUCGGGAGGAUGGGCAC | 10848 | AGGGGTCGGGAGGATGGGCAC |
| 5184 | AGGGGUGGCCCCAGGCAGCCG | 10849 | AGGGGTGGCCCCAGGCAGCCG |
| 5185 | AGGGGUGGGCCAGCCUCUUUU | 10850 | AGGGGTGGGCCAGCCTCTTTT |
| 5186 | AGGGGUUUGGCUUAGAGAUUG | 10851 | AGGGGTTTGGCTTAGAGATTG |
| 5187 | AGGGUCUUCAUCUGUUGCCCA | 10852 | AGGGTCTTCATCTGTTGCCCA |
| 5188 | AGGGUGACCAGUGACUCAGGC | 10853 | AGGGTGACCAGTGACTCAGGC |
| 5189 | AGGGUGAUGCAUUCGCCGCUG | 10854 | AGGGTGATGCATTCGCCGCTG |
| 5190 | AGGGUGAUGCCAUUGGGCCAC | 10855 | AGGGTGATGCCATTGGGCCAC |
| 5191 | AGGGUGGCCGGGGGCCCCUCU | 10856 | AGGGTGGCCGGGGGCCCCTCT |
| 5192 | AGGGUGGUCCUCUCACACCAG | 10857 | AGGGTGGTCCTCTCACACCAG |
| 5193 | AGGGUUCUGGGCAGGGACGGG | 10858 | AGGGTTCTGGGCAGGGACGGG |
| 5194 | AGGGUUUGCAAAAAUAAAUAG | 10859 | AGGGTTTGCAAAAATAAATAG |
| 5195 | AGGUAAACACGUUGACUCCUG | 10860 | AGGTAAACACGTTGACTCCTG |
| 5196 | AGGUAAACUUGGGCGAGUGGG | 10861 | AGGTAAACTTGGGCGAGTGGG |
| 5197 | AGGUAACCGGGUGUCUCAGGC | 10862 | AGGTAACCGGGTGTCTCAGGC |
| 5198 | AGGUACAAACCCACCUCGAAA | 10863 | AGGTACAAACCCACCTCGAAA |
| 5199 | AGGUAGGCGAUGGAGCCCACA | 10864 | AGGTAGGCGATGGAGCCCACA |
| 5200 | AGGUCAGACCAGUAGAUUCUA | 10865 | AGGTCAGACCAGTAGATTCTA |
| 5201 | AGGUCAUUGCAGACGUGGGAA | 10866 | AGGTCATTGCAGACGTGGGAA |
| 5202 | AGGUCGGGUGGUUGUGUGCUG | 10867 | AGGTCGGGTGGTTGTGTGCTG |
| 5203 | AGGUCUCAGGAAGGGUUCUGG | 10868 | AGGTCTCAGGAAGGGTTCTGG |
| 5204 | AGGUGACAGACAAGCACGUCU | 10869 | AGGTGACAGACAAGCACGTCT |
| 5205 | AGGUGAGCACCGGGCAGGAGG | 10870 | AGGTGAGCACCGGGCAGGAGG |
| 5206 | AGGUGCCCGCCACCACGCCCG | 10871 | AGGTGCCCGCCACCACGCCCG |
| 5207 | AGGUGGAGCUGUUGCACUGGA | 10872 | AGGTGGAGCTGTTGCACTGGA |
| 5208 | AGGUGGCCACAGCGCAGUUUU | 10873 | AGGTGGCCACAGCGCAGTTTT |
| 5209 | AGGUGGCCACAGGACAGCCUU | 10874 | AGGTGGCCACAGGACAGCCTT |
| 5210 | AGGUGGGCGGACAUAAAGACG | 10875 | AGGTGGGCGGACATAAAGACG |
| 5211 | AGGUGGGGACGGAGGUGUCGC | 10876 | AGGTGGGGACGGAGGTGTCGC |
| 5212 | AGGUGUCAUCCUGGUCCCCCC | 10877 | AGGTGTCATCCTGGTCCCCCC |
| 5213 | AGGUGUCGCCACAGAGCACAG | 10878 | AGGTGTCGCCACAGAGCACAG |
| 5214 | AGGUGUCGGGAACAGGUCGGG | 10879 | AGGTGTCGGGAACAGGTCGGG |
| 5215 | AGGUGUCGGGAUCCUGACACU | 10880 | AGGTGTCGGGATCCTGACACT |
| 5216 | AGGUUCACGCAGAGCUGGCUG | 10881 | AGGTTCACGCAGAGCTGGCTG |
| 5217 | AGGUUGGGGAUGAGGCUGGUG | 10882 | AGGTTGGGGATGAGGCTGGTG |
| 5218 | AGGUUGUGGAAGAGAACCAUA | 10883 | AGGTTGTGGAAGAGAACCATA |
| 5219 | AGGUUUUCAGCCAACAAGUUG | 10884 | AGGTTTTCAGCCAACAAGTTG |
| 5220 | AGUAACACGGCGAUUUCAGAA | 10885 | AGTAACACGGCGATTTCAGAA |
| 5221 | AGUAAGACAUUCAGGCUACAA | 10886 | AGTAAGACATTCAGGCTACAA |
| 5222 | AGUACAUGAAGCCAUGAACAG | 10887 | AGTACATGAAGCCATGAACAG |
| 5223 | AGUAGAGACGGGGUUUCACGU | 10888 | AGTAGAGACGGGGTTTCACGT |
| 5224 | AGUAGAGGCGGCCACUGAGGA | 10889 | AGTAGAGGCGGCCACTGAGGA |
| 5225 | AGUAGAUGUCCACACCAUUCA | 10890 | AGTAGATGTCCACACCATTCA |
| 5226 | AGUAGAUGUUGCUGUGGAUCC | 10891 | AGTAGATGTTGCTGTGGATCC |
| 5227 | AGUAGAUUCUAUUGCUGGCCA | 10892 | AGTAGATTCTATTGCTGGCCA |
| 5228 | AGUAGCUGGGACUACAGGUGC | 10893 | AGTAGCTGGGACTACAGGTGC |
| 5229 | AGUAGCUGGGAUCACAGGCAC | 10894 | AGTAGCTGGGATCACAGGCAC |
| 5230 | AGUAGGUUUUCAGCCAACAAG | 10895 | AGTAGGTTTTCAGCCAACAAG |
| 5231 | AGUCAACCCAGUAGAGGCGGC | 10896 | AGTCAACCCAGTAGAGGCGGC |
| 5232 | AGUCACAGACGAACUGCCGAG | 10897 | AGTCACAGACGAACTGCCGAG |
| 5233 | AGUCACCAGCGAGUAGAUGUC | 10898 | AGTCACCAGCGAGTAGATGTC |
| 5234 | AGUCACUGAACAAAUACAGCA | 10899 | AGTCACTGAACAAATACAGCA |
| 5235 | AGUCAGUCCAGUACAUGAAGC | 10900 | AGTCAGTCCAGTACATGAAGC |
| 5236 | AGUCAUAUUCCCGGUCACACU | 10901 | AGTCATATTCCCGGTCACACT |
| 5237 | AGUCCACAGCCAGCCCGUCGG | 10902 | AGTCCACAGCCAGCCCGTCGG |
| 5238 | AGUCCACUUGGCCAUCGCACC | 10903 | AGTCCACTTGGCCATCGCACC |
| 5239 | AGUCCAGUACAUGAAGCCAUG | 10904 | AGTCCAGTACATGAAGCCATG |
| 5240 | AGUCCAGUUCGUGCAAAUAAU | 10905 | AGTCCAGTTCGTGCAAATAAT |
| 5241 | AGUCCCCGCCGCGGCGAGGAG | 10906 | AGTCCCCGCCGCGGCGAGGAG |
| 5242 | AGUCCCCGGAUUUGCAGGUGA | 10907 | AGTCCCCGGATTTGCAGGTGA |
| 5243 | AGUCCCGGCAGUCUCUAGCCA | 10908 | AGTCCCGGCAGTCTCTAGCCA |
| 5244 | AGUCCCGGUCAACCUGCCCCU | 10909 | AGTCCCGGTCAACCTGCCCCT |
| 5245 | AGUCCCGGUCUGAGUCACAGA | 10910 | AGTCCCGGTCTGAGTCACAGA |
| 5246 | AGUCCCGUCAAACGAUCCAGA | 10911 | AGTCCCGTCAAACGATCCAGA |
| 5247 | AGUCGGGGCCACCAUCACAGC | 10912 | AGTCGGGGCCACCATCACAGC |
| 5248 | AGUCGGGGUCGUUGUCGCAGG | 10913 | AGTCGGGGTCGTTGTCGCAGG |
| 5249 | AGUCGGUCCAGUAGAUGUUGC | 10914 | AGTCGGTCCAGTAGATGTTGC |
| 5250 | AGUCUCGCUCUGUCGCCCAGG | 10915 | AGTCTCGCTCTGTCGCCCAGG |
| 5251 | AGUCUCUAGCCAUGUUGCAGA | 10916 | AGTCTCTAGCCATGTTGCAGA |
| 5252 | AGUCUGACGGAGGCUGUCACU | 10917 | AGTCTGACGGAGGCTGTCACT |
| 5253 | AGUGAAGUUUGGAGUCAACCC | 10918 | AGTGAAGTTTGGAGTCAACCC |
| 5254 | AGUGAAUGGCUUGGAGGCAUU | 10919 | AGTGAATGGCTTGGAGGCATT |
| 5255 | AGUGACACCCAUCUCCCAGAA | 10920 | AGTGACACCCATCTCCCAGAA |
| 5256 | AGUGACUCAGGCUCUGCCUCU | 10921 | AGTGACTCAGGCTCTGCCTCT |
| 5257 | AGUGAUAACAGUUUCAACCAA | 10922 | AGTGATAACAGTTTCAACCAA |
| 5258 | AGUGAUCCAGCUCAGCCUCCC | 10923 | AGTGATCCAGCTCAGCCTCCC |
| 5259 | AGUGCAAAAUAAUAACACAAA | 10924 | AGTGCAAAATAATAACACAAA |
| 5260 | AGUGCAAACAUAACAAAAAAU | 10925 | AGTGCAAACATAACAAAAAAT |
| 5261 | AGUGCAACAGUAACACGGCGA | 10926 | AGTGCAACAGTAACACGGCGA |
| 5262 | AGUGCAAGGAGACCACGGGAA | 10927 | AGTGCAAGGAGACCACGGGAA |
| 5263 | AGUGCAGUGGCUCGAUCAUGG | 10928 | AGTGCAGTGGCTCGATCATGG |
| 5264 | AGUGCCCAGGACAGAGUCGGU | 10929 | AGTGCCCAGGACAGAGTCGGT |
| 5265 | AGUGCUGGGAUUACAGGCGUG | 10930 | AGTGCTGGGATTACAGGCGTG |
| 5266 | AGUGGAACUCGAAGGCCGAGC | 10931 | AGTGGAACTCGAAGGCCGAGC |
| 5267 | AGUGGAAGCACUAGGUGGGCG | 10932 | AGTGGAAGCACTAGGTGGGCG |
| 5268 | AGUGGAUGCACUCGCCACUUA | 10933 | AGTGGATGCACTCGCCACTTA |
| 5269 | AGUGGCAUAAUCAUAGCUCAC | 10934 | AGTGGCATAATCATAGCTCAC |
| 5270 | AGUGGCAUCAUUUGGUGAAUG | 10935 | AGTGGCATCATTTGGTGAATG |
| 5271 | AGUGGCGCAAUCUCGGCUCAC | 10936 | AGTGGCGCAATCTCGGCTCAC |
| 5272 | AGUGGCUCGAUCAUGGCUCAC | 10937 | AGTGGCTCGATCATGGCTCAC |
| 5273 | AGUGGGGGUUGAUCUGCGGGG | 10938 | AGTGGGGGTTGATCTGCGGGG |
| 5274 | AGUGGGUAGGGGUCGGGAGGA | 10939 | AGTGGGTAGGGGTCGGGAGGA |
| 5275 | AGUGGUUUAAAAAGUGACACC | 10940 | AGTGGTTTAAAAAGTGACACC |
| 5276 | AGUGUACAACUCUGAACUGAG | 10941 | AGTGTACAACTCTGAACTGAG |
| 5277 | AGUGUCACAUUAACGCAGCCA | 10942 | AGTGTCACATTAACGCAGCCA |
| 5278 | AGUGUCAGACAGCCUUGCUCG | 10943 | AGTGTCAGACAGCCTTGCTCG |
| 5279 | AGUGUCCCGACCCGGAUCACG | 10944 | AGTGTCCCGACCCGGATCACG |
| 5280 | AGUGUUUUGUCCCUGGGGACA | 10945 | AGTGTTTTGTCCCTGGGGACA |
| 5281 | AGUUCACUCCUCUUGGCUGGG | 10946 | AGTTCACTCCTCTTGGCTGGG |
| 5282 | AGUUCCCCAGUCAGUCCAGUA | 10947 | AGTTCCCCAGTCAGTCCAGTA |
| 5283 | AGUUCGUGCAAAUAAUCUUUG | 10948 | AGTTCGTGCAAATAATCTTTG |
| 5284 | AGUUCUCGAUAUUCACGAUCA | 10949 | AGTTCTCGATATTCACGATCA |
| 5285 | AGUUCUUCCAUAGAAGGAAGA | 10950 | AGTTCTTCCATAGAAGGAAGA |
| 5286 | AGUUGACAUCGGAACCUGUGA | 10951 | AGTTGACATCGGAACCTGTGA |
| 5287 | AGUUGAUGCUGUUGAUGUUCU | 10952 | AGTTGATGCTGTTGATGTTCT |
| 5288 | AGUUGUAUUUCUGAAAUAGGG | 10953 | AGTTGTATTTCTGAAATAGGG |
| 5289 | AGUUUAGGGGUUUGGCUUAGA | 10954 | AGTTTAGGGGTTTGGCTTAGA |
| 5290 | AGUUUCAACCAAUAUACAAAG | 10955 | AGTTTCAACCAATATACAAAG |
| 5291 | AGUUUCCAUCAGAGCACUGGA | 10956 | AGTTTCCATCAGAGCACTGGA |
| 5292 | AGUUUGGAGUCAACCCAGUAG | 10957 | AGTTTGGAGTCAACCCAGTAG |
| 5293 | AGUUUGGUCUCGGUGCCACCA | 10958 | AGTTTGGTCTCGGTGCCACCA |
| 5294 | AGUUUUCCUCGUCAGAUUUGU | 10959 | AGTTTTCCTCGTCAGATTTGT |
| 5295 | AUAAAACAAAGCUCUGGCAGG | 10960 | ATAAAACAAAGCTCTGGCAGG |
| 5296 | AUAAAACAAGGCCGGCGAGGU | 10961 | ATAAAACAAGGCCGGCGAGGT |
| 5297 | AUAAAGAAGAGGCAUUUCAGA | 10962 | ATAAAGAAGAGGCATTTCAGA |
| 5298 | AUAAAGACGACAAUGGCAGUU | 10963 | ATAAAGACGACAATGGCAGTT |
| 5299 | AUAAAUAGAUUUUAUAUAUAU | 10964 | ATAAATAGATTTTATATATAT |
| 5300 | AUAAAUAUAUAAAACAAAGCU | 10965 | ATAAATATATAAAACAAAGCT |
| 5301 | AUAAAUAUUAAGGGUGACCAG | 10966 | ATAAATATTAAGGGTGACCAG |
| 5302 | AUAACAAAAAAUCCCCACUAU | 10967 | ATAACAAAAAATCCCCACTAT |
| 5303 | AUAACACAAAUGCCAAAUGUA | 10968 | ATAACACAAATGCCAAATGTA |
| 5304 | AUAACAGUUUCAACCAAUAUA | 10969 | ATAACAGTTTCAACCAATATA |
| 5305 | AUAACCCCCUACACAGGGCCC | 10970 | ATAACCCCCTACACAGGGCCC |
| 5306 | AUAACGGAGGGCGACUCGUGU | 10971 | ATAACGGAGGGCGACTCGTGT |
| 5307 | AUAACGUUUUCCUCUUCACGC | 10972 | ATAACGTTTTCCTCTTCACGC |
| 5308 | AUAAGUGAUAACAGUUUCAAC | 10973 | ATAAGTGATAACAGTTTCAAC |
| 5309 | AUAAUAACACAAAUGCCAAAU | 10974 | ATAATAACACAAATGCCAAAT |
| 5310 | AUAAUCAUAGCUCACGACAGC | 10975 | ATAATCATAGCTCACGACAGC |
| 5311 | AUAAUCUUUGUACAUAAAGAA | 10976 | ATAATCTTTGTACATAAAGAA |
| 5312 | AUACAAAGUGCAAACAUAACA | 10977 | ATACAAAGTGCAAACATAACA |
| 5313 | AUACAACGGGGACAUCAUUCU | 10978 | ATACAACGGGGACATCATTCT |
| 5314 | AUACAGCAACCAGGGUUUGCA | 10979 | ATACAGCAACCAGGGTTTGCA |
| 5315 | AUACAGUGGUUUAAAAAGUGA | 10980 | ATACAGTGGTTTAAAAAGTGA |
| 5316 | AUACAUACAACGGGGACAUCA | 10981 | ATACATACAACGGGGACATCA |
| 5317 | AUACCCAUCAACGACAAGAUU | 10982 | ATACCCATCAACGACAAGATT |
| 5318 | AUACUGGCAGCCGCCAUUGCU | 10983 | ATACTGGCAGCCGCCATTGCT |
| 5319 | AUACUUUGUCCUCAAAGACGG | 10984 | ATACTTTGTCCTCAAAGACGG |
| 5320 | AUAGAAGAGGUAAACACGUUG | 10985 | ATAGAAGAGGTAAACACGTTG |
| 5321 | AUAGAAGGAAGACCCCCAGGC | 10986 | ATAGAAGGAAGACCCCCAGGC |
| 5322 | AUAGAAGUGGAAGCACUAGGU | 10987 | ATAGAAGTGGAAGCACTAGGT |
| 5323 | AUAGACGGGGUUGUCAAAGUU | 10988 | ATAGACGGGGTTGTCAAAGTT |
| 5324 | AUAGAUUUUAUAUAUAUGUGU | 10989 | ATAGATTTTATATATATGTGT |
| 5325 | AUAGCUCACGACAGCCUCAAA | 10990 | ATAGCTCACGACAGCCTCAAA |
| 5326 | AUAGGAAGAGACGCCGUGGGC | 10991 | ATAGGAAGAGACGCCGTGGGC |
| 5327 | AUAGGGUCUUCAUCUGUUGCC | 10992 | ATAGGGTCTTCATCTGTTGCC |
| 5328 | AUAGUCACUGAACAAAUACAG | 10993 | ATAGTCACTGAACAAATACAG |
| 5329 | AUAGUUGUAUUUCUGAAAUAG | 10994 | ATAGTTGTATTTCTGAAATAG |
| 5330 | AUAUAAAACAAAGCUCUGGCA | 10995 | ATATAAAACAAAGCTCTGGCA |
| 5331 | AUAUAAGUGAUAACAGUUUCA | 10996 | ATATAAGTGATAACAGTTTCA |
| 5332 | AUAUACAAAGUGCAAACAUAA | 10997 | ATATACAAAGTGCAAACATAA |
| 5333 | AUAUAUAAAACAAAGCUCUGG | 10998 | ATATATAAAACAAAGCTCTGG |
| 5334 | AUAUAUAAGUGAUAACAGUUU | 10999 | ATATATAAGTGATAACAGTTT |
| 5335 | AUAUAUAUAAGUGAUAACAGU | 11000 | ATATATATAAGTGATAACAGT |
| 5336 | AUAUAUAUAUAAGUGAUAACA | 11001 | ATATATATATAAGTGATAACA |
| 5337 | AUAUAUAUAUAUAAGUGAUAA | 11002 | ATATATATATATAAGTGATAA |
| 5338 | AUAUAUAUAUAUAUAAGUGAU | 11003 | ATATATATATATATAAGTGAT |
| 5339 | AUAUAUAUGUGUGUAUAUAUA | 11004 | ATATATATGTGTGTATATATA |
| 5340 | AUAUAUGUGUGUAUAUAUAUA | 11005 | ATATATGTGTGTATATATATA |
| 5341 | AUAUCCUCUGGGGACAGUAGG | 11006 | ATATCCTCTGGGGACAGTAGG |
| 5342 | AUAUCGCACUCUUUGAUGGGU | 11007 | ATATCGCACTCTTTGATGGGT |
| 5343 | AUAUCUGUCCAAAAUACUUUG | 11008 | ATATCTGTCCAAAATACTTTG |
| 5344 | AUAUCUUCGCAUCUUCGCUGG | 11009 | ATATCTTCGCATCTTCGCTGG |
| 5345 | AUAUGUGUGUAUAUAUAUAUA | 11010 | ATATGTGTGTATATATATATA |
| 5346 | AUAUUAAGGGUGACCAGUGAC | 11011 | ATATTAAGGGTGACCAGTGAC |
| 5347 | AUAUUCACGAUCACGGCUUUG | 11012 | ATATTCACGATCACGGCTTTG |
| 5348 | AUAUUCCCGGUCACACUGCCG | 11013 | ATATTCCCGGTCACACTGCCG |
| 5349 | AUCAACGACAAGAUUGGGGAA | 11014 | ATCAACGACAAGATTGGGGAA |
| 5350 | AUCACAGCGCCAGCUGGAGUG | 11015 | ATCACAGCGCCAGCTGGAGTG |
| 5351 | AUCACAGGCACGUGCCACCGU | 11016 | ATCACAGGCACGTGCCACCGT |
| 5352 | AUCACAGUAAGACAUUCAGGC | 11017 | ATCACAGTAAGACATTCAGGC |
| 5353 | AUCACGACCUGCUGUGUCCUA | 11018 | ATCACGACCTGCTGTGTCCTA |
| 5354 | AUCACGGCUUUGAAGUCCCGG | 11019 | ATCACGGCTTTGAAGTCCCGG |
| 5355 | AUCACGGUUCCCUGCACUGGG | 11020 | ATCACGGTTCCCTGCACTGGG |
| 5356 | AUCAGAGCACUGGAAUUCGUC | 11021 | ATCAGAGCACTGGAATTCGTC |
| 5357 | AUCAGAGCCAUCCUGGCACUC | 11022 | ATCAGAGCCATCCTGGCACTC |
| 5358 | AUCAGUGCAACAGUAACACGG | 11023 | ATCAGTGCAACAGTAACACGG |
| 5359 | AUCAUAGCUCACGACAGCCUC | 11024 | ATCATAGCTCACGACAGCCTC |
| 5360 | AUCAUGGCUCACUGCAACCUC | 11025 | ATCATGGCTCACTGCAACCTC |
| 5361 | AUCAUUCUCCCAAAAAGCGUU | 11026 | ATCATTCTCCCAAAAAGCGTT |
| 5362 | AUCAUUCUCUGGGACAGGUCA | 11027 | ATCATTCTCTGGGACAGGTCA |
| 5363 | AUCAUUUCCGACGCCAUGAAU | 11028 | ATCATTTCCGACGCCATGAAT |
| 5364 | AUCAUUUGGUGAAUGAGUUUG | 11029 | ATCATTTGGTGAATGAGTTTG |
| 5365 | AUCCACCACGAUGGCCCUUGG | 11030 | ATCCACCACGATGGCCCTTGG |
| 5366 | AUCCACCCGCCUCGGCCUCCC | 11031 | ATCCACCCGCCTCGGCCTCCC |
| 5367 | AUCCAGACUGGAGGAUUUAUU | 11032 | ATCCAGACTGGAGGATTTATT |
| 5368 | AUCCAGCUCAGCCUCCCAAAG | 11033 | ATCCAGCTCAGCCTCCCAAAG |
| 5369 | AUCCAGUCCACAGCCAGCCCG | 11034 | ATCCAGTCCACAGCCAGCCCG |
| 5370 | AUCCCAACACACACGACAGAA | 11035 | ATCCCAACACACACGACAGAA |
| 5371 | AUCCCAACCCAAGCCAUUGCA | 11036 | ATCCCAACCCAAGCCATTGCA |
| 5372 | AUCCCAUCCCAACACACACGA | 11037 | ATCCCATCCCAACACACACGA |
| 5373 | AUCCCCACUAUCUGUACAGGG | 11038 | ATCCCCACTATCTGTACAGGG |
| 5374 | AUCCCCUCCCCUUUCAUCUUC | 11039 | ATCCCCTCCCCTTTCATCTTC |
| 5375 | AUCCCUUGUGACAUCUUCACG | 11040 | ATCCCTTGTGACATCTTCACG |
| 5376 | AUCCCUUUCCUGGCUUAAGGA | 11041 | ATCCCTTTCCTGGCTTAAGGA |
| 5377 | AUCCGAGCCAUCUUCGCAGUC | 11042 | ATCCGAGCCATCTTCGCAGTC |
| 5378 | AUCCGCAACAGAGACAGUGCC | 11043 | ATCCGCAACAGAGACAGTGCC |
| 5379 | AUCCUCCAAGAUGGUCUUCCG | 11044 | ATCCTCCAAGATGGTCTTCCG |
| 5380 | AUCCUCCAGACUGACCAUCGA | 11045 | ATCCTCCAGACTGACCATCGA |
| 5381 | AUCCUCCAGACUGACCAUCUG | 11046 | ATCCTCCAGACTGACCATCTG |
| 5382 | AUCCUCCCACCUCAGCCUCGC | 11047 | ATCCTCCCACCTCAGCCTCGC |
| 5383 | AUCCUCCUGCCUCAGCCUCCC | 11048 | ATCCTCCTGCCTCAGCCTCCC |
| 5384 | AUCCUCUGGGGACAGUAGGUU | 11049 | ATCCTCTGGGGACAGTAGGTT |
| 5385 | AUCCUCUGUGGUCUUCUGAUA | 11050 | ATCCTCTGTGGTCTTCTGATA |
| 5386 | AUCCUGACACUCAUCGAUAUC | 11051 | ATCCTGACACTCATCGATATC |
| 5387 | AUCCUGGCACUCAGCGCUGCC | 11052 | ATCCTGGCACTCAGCGCTGCC |
| 5388 | AUCCUGGUCCCCCCGGGCCUG | 11053 | ATCCTGGTCCCCCCGGGCCTG |
| 5389 | AUCCUUCACGAGGAAAGGAAG | 11054 | ATCCTTCACGAGGAAAGGAAG |
| 5390 | AUCGAGGGGUAGCUGUAGCCG | 11055 | ATCGAGGGGTAGCTGTAGCCG |
| 5391 | AUCGAUAUCGCACUCUUUGAU | 11056 | ATCGATATCGCACTCTTTGAT |
| 5392 | AUCGAUAUCUUCGCAUCUUCG | 11057 | ATCGATATCTTCGCATCTTCG |
| 5393 | AUCGAUGCUUGAGAUGGAGUG | 11058 | ATCGATGCTTGAGATGGAGTG |
| 5394 | AUCGCACCUCCAGAACUGAGG | 11059 | ATCGCACCTCCAGAACTGAGG |
| 5395 | AUCGCACUCUUUGAUGGGUUC | 11060 | ATCGCACTCTTTGATGGGTTC |
| 5396 | AUCGCAGACCCACUUGUAGGA | 11061 | ATCGCAGACCCACTTGTAGGA |
| 5397 | AUCGCUCAUGUCCUUGCAGUC | 11062 | ATCGCTCATGTCCTTGCAGTC |
| 5398 | AUCGGAACCUGUGAGGCGGUU | 11063 | ATCGGAACCTGTGAGGCGGTT |
| 5399 | AUCGGGGCUGGUGGGGCGAGG | 11064 | ATCGGGGCTGGTGGGGCGAGG |
| 5400 | AUCGUGGCAGCGAAACUCGUC | 11065 | ATCGTGGCAGCGAAACTCGTC |
| 5401 | AUCUAGGGUGAUGCCAUUGGG | 11066 | ATCTAGGGTGATGCCATTGGG |
| 5402 | AUCUCCACCGUGGUGAGCCCA | 11067 | ATCTCCACCGTGGTGAGCCCA |
| 5403 | AUCUCCCAGAAGCCACUCAUA | 11068 | ATCTCCCAGAAGCCACTCATA |
| 5404 | AUCUCCUGACCUCAUGAUCCA | 11069 | ATCTCCTGACCTCATGATCCA |
| 5405 | AUCUCGCUUUGUUGCUCAGGC | 11070 | ATCTCGCTTTGTTGCTCAGGC |
| 5406 | AUCUCGGCUCACUGCAAGCUC | 11071 | ATCTCGGCTCACTGCAAGCTC |
| 5407 | AUCUCGGCUGCAGACCAAGGA | 11072 | ATCTCGGCTGCAGACCAAGGA |
| 5408 | AUCUGACCAGUCCCGGCAGUC | 11073 | ATCTGACCAGTCCCGGCAGTC |
| 5409 | AUCUGCGGGGCAGGGAGGCAC | 11074 | ATCTGCGGGGCAGGGAGGCAC |
| 5410 | AUCUGCUGUUGUGUGUUGAAA | 11075 | ATCTGCTGTTGTGTGTTGAAA |
| 5411 | AUCUGUACAGGGACGCAUUUA | 11076 | ATCTGTACAGGGACGCATTTA |
| 5412 | AUCUGUCCAAAAUACUUUGUC | 11077 | ATCTGTCCAAAATACTTTGTC |
| 5413 | AUCUGUCGCCCACGUGGCUGA | 11078 | ATCTGTCGCCCACGTGGCTGA |
| 5414 | AUCUGUCGCCCACUGCAGUCC | 11079 | ATCTGTCGCCCACTGCAGTCC |
| 5415 | AUCUGUCUCGAGGGGUAGCUG | 11080 | ATCTGTCTCGAGGGGTAGCTG |
| 5416 | AUCUGUUGCCCAGGCUGGAGU | 11081 | ATCTGTTGCCCAGGCTGGAGT |
| 5417 | AUCUUAAGGUCAUUGCAGACG | 11082 | ATCTTAAGGTCATTGCAGACG |
| 5418 | AUCUUCACGCGGGAGUCUGAC | 11083 | ATCTTCACGCGGGAGTCTGAC |
| 5419 | AUCUUCCAUGUUUGAGGAUUG | 11084 | ATCTTCCATGTTTGAGGATTG |
| 5420 | AUCUUCCUGACCUCGUGCCGG | 11085 | ATCTTCCTGACCTCGTGCCGG |
| 5421 | AUCUUCGCAGUCGGGGUCGUU | 11086 | ATCTTCGCAGTCGGGGTCGTT |
| 5422 | AUCUUCGCAUCUUCGCUGGGC | 11087 | ATCTTCGCATCTTCGCTGGGC |
| 5423 | AUCUUCGCUGGGCCACCAGCU | 11088 | ATCTTCGCTGGGCCACCAGCT |
| 5424 | AUCUUGGCGGGAGUUCCCCAG | 11089 | ATCTTGGCGGGAGTTCCCCAG |
| 5425 | AUCUUUGUACAUAAAGAAGAG | 11090 | ATCTTTGTACATAAAGAAGAG |
| 5426 | AUGAACAGGAUCCACCACGAU | 11091 | ATGAACAGGATCCACCACGAT |
| 5427 | AUGAAGCCAUGAACAGGAUCC | 11092 | ATGAAGCCATGAACAGGATCC |
| 5428 | AUGAAUAAAUAUAUAAAACAA | 11093 | ATGAATAAATATATAAAACAA |
| 5429 | AUGAAUCCCUUUCCUGGCUUA | 11094 | ATGAATCCCTTTCCTGGCTTA |
| 5430 | AUGACGGUGUCAUAGGAAGAG | 11095 | ATGACGGTGTCATAGGAAGAG |
| 5431 | AUGAGGCAGGGCCAGGGGGUC | 11096 | ATGAGGCAGGGCCAGGGGGTC |
| 5432 | AUGAGGCUGGUGUACUCGCUC | 11097 | ATGAGGCTGGTGTACTCGCTC |
| 5433 | AUGAGUUUGGUCUCGGUGCCA | 11098 | ATGAGTTTGGTCTCGGTGCCA |
| 5434 | AUGAUAUCUGUCCAAAAUACU | 11099 | ATGATATCTGTCCAAAATACT |
| 5435 | AUGAUCCACCCGCCUCGGCCU | 11100 | ATGATCCACCCGCCTCGGCCT |
| 5436 | AUGAUUCGGGGGUGACAGGGC | 11101 | ATGATTCGGGGGTGACAGGGC |
| 5437 | AUGCACCAUGCAUGGUUUUUU | 11102 | ATGCACCATGCATGGTTTTTT |
| 5438 | AUGCACUCGCCACUUAGGCAG | 11103 | ATGCACTCGCCACTTAGGCAG |
| 5439 | AUGCACUUCCCAUCGUGGCAG | 11104 | ATGCACTTCCCATCGTGGCAG |
| 5440 | AUGCAGCGGUUGACACGGCCC | 11105 | ATGCAGCGGTTGACACGGCCC |
| 5441 | AUGCAGGUGGAGCUGUUGCAC | 11106 | ATGCAGGTGGAGCTGTTGCAC |
| 5442 | AUGCAGUUUCCAUCAGAGCAC | 11107 | ATGCAGTTTCCATCAGAGCAC |
| 5443 | AUGCAUGGUUUUUUUUUGUUU | 11108 | ATGCATGGTTTTTTTTTGTTT |
| 5444 | AUGCAUUCGCCGCUGUGACAC | 11109 | ATGCATTCGCCGCTGTGACAC |
| 5445 | AUGCAUUUCCCGUCUUGGCAC | 11110 | ATGCATTTCCCGTCTTGGCAC |
| 5446 | AUGCCAAAUGUACACAGUGUA | 11111 | ATGCCAAATGTACACAGTGTA |
| 5447 | AUGCCAUUGGGCCACUGAAUG | 11112 | ATGCCATTGGGCCACTGAATG |
| 5448 | AUGCCGUCCGGGCAGGCGCAG | 11113 | ATGCCGTCCGGGCAGGCGCAG |
| 5449 | AUGCCUGAGCUCAAACCAUCC | 11114 | ATGCCTGAGCTCAAACCATCC |
| 5450 | AUGCCUGGCCAGAGGCCAUGG | 11115 | ATGCCTGGCCAGAGGCCATGG |
| 5451 | AUGCUCGCAGCCUCUGCCAGG | 11116 | ATGCTCGCAGCCTCTGCCAGG |
| 5452 | AUGCUGUUGAUGUUCUUAAGC | 11117 | ATGCTGTTGATGTTCTTAAGC |
| 5453 | AUGCUUGAGAUGGAGUGAAGU | 11118 | ATGCTTGAGATGGAGTGAAGT |
| 5454 | AUGCUUUGGUCUUCUCUGUCU | 11119 | ATGCTTTGGTCTTCTCTGTCT |
| 5455 | AUGGAAGUGGGUAGGGGUCGG | 11120 | ATGGAAGTGGGTAGGGGTCGG |
| 5456 | AUGGACAGAGCCCUCACGCUA | 11121 | ATGGACAGAGCCCTCACGCTA |
| 5457 | AUGGAGCCCACAGCCUUGCAG | 11122 | ATGGAGCCCACAGCCTTGCAG |
| 5458 | AUGGAGGUGUCAUCCUGGUCC | 11123 | ATGGAGGTGTCATCCTGGTCC |
| 5459 | AUGGAGUGAAGUUUGGAGUCA | 11124 | ATGGAGTGAAGTTTGGAGTCA |
| 5460 | AUGGAUGCAGUUUCCAUCAGA | 11125 | ATGGATGCAGTTTCCATCAGA |
| 5461 | AUGGCAGUUCUCGAUAUUCAC | 11126 | ATGGCAGTTCTCGATATTCAC |
| 5462 | AUGGCCCUUGGCUUGGAGCCG | 11127 | ATGGCCCTTGGCTTGGAGCCG |
| 5463 | AUGGCUCACUGCAACCUCCAC | 11128 | ATGGCTCACTGCAACCTCCAC |
| 5464 | AUGGCUUCGUUGAUGAUAUCU | 11129 | ATGGCTTCGTTGATGATATCT |
| 5465 | AUGGCUUGGAGGCAUUUGCAU | 11130 | ATGGCTTGGAGGCATTTGCAT |
| 5466 | AUGGGAUCUCGCUUUGUUGCU | 11131 | ATGGGATCTCGCTTTGTTGCT |
| 5467 | AUGGGCACCAGGCUGGUGUCC | 11132 | ATGGGCACCAGGCTGGTGTCC |
| 5468 | AUGGGCACUGUCCGAAGCCUG | 11133 | ATGGGCACTGTCCGAAGCCTG |
| 5469 | AUGGGCUGCUGAUGCACCAUG | 11134 | ATGGGCTGCTGATGCACCATG |
| 5470 | AUGGGGAGGACAAUGGACAGA | 11135 | ATGGGGAGGACAATGGACAGA |
| 5471 | AUGGGGUCUUACUAUGUUGCC | 11136 | ATGGGGTCTTACTATGTTGCC |
| 5472 | AUGGGUUCAUCUGACCAGUCC | 11137 | ATGGGTTCATCTGACCAGTCC |
| 5473 | AUGGUCUCGAUCUCCUGACCU | 11138 | ATGGTCTCGATCTCCTGACCT |
| 5474 | AUGGUCUUCCGGUUGCCCCCG | 11139 | ATGGTCTTCCGGTTGCCCCCG |
| 5475 | AUGGUUUUUUUUUGUUUUUUU | 11140 | ATGGTTTTTTTTTGTTTTTTT |
| 5476 | AUGUACACAGUGUACAACUCU | 11141 | ATGTACACAGTGTACAACTCT |
| 5477 | AUGUCCACACCAUUCAGGCCC | 11142 | ATGTCCACACCATTCAGGCCC |
| 5478 | AUGUCCCUGGCCAGCAGCAUG | 11143 | ATGTCCCTGGCCAGCAGCATG |
| 5479 | AUGUCCUUGCAGUCAUAUUCC | 11144 | ATGTCCTTGCAGTCATATTCC |
| 5480 | AUGUCGCGGAGGCGGCUCCGG | 11145 | ATGTCGCGGAGGCGGCTCCGG |
| 5481 | AUGUCUCCUGGGUGGCCACUG | 11146 | ATGTCTCCTGGGTGGCCACTG |
| 5482 | AUGUCUCUGCUGAUGACGGUG | 11147 | ATGTCTCTGCTGATGACGGTG |
| 5483 | AUGUGGACCUCAUCCUCUGUG | 11148 | ATGTGGACCTCATCCTCTGTG |
| 5484 | AUGUGUGUAUAUAUAUAUAUA | 11149 | ATGTGTGTATATATATATATA |
| 5485 | AUGUUCACGCCACGUCAUCCU | 11150 | ATGTTCACGCCACGTCATCCT |
| 5486 | AUGUUCUUAAGCCGCCAGUUC | 11151 | ATGTTCTTAAGCCGCCAGTTC |
| 5487 | AUGUUGCAGACUUUGUCCAGG | 11152 | ATGTTGCAGACTTTGTCCAGG |
| 5488 | AUGUUGCCCAGGCUGGAAAGC | 11153 | ATGTTGCCCAGGCTGGAAAGC |
| 5489 | AUGUUGCUGUGGAUCCAGUCC | 11154 | ATGTTGCTGTGGATCCAGTCC |
| 5490 | AUGUUUGAGGAUUGUGUCACG | 11155 | ATGTTTGAGGATTGTGTCACG |
| 5491 | AUGUUUUAAACACAUACCCAU | 11156 | ATGTTTTAAACACATACCCAT |
| 5492 | AUGUUUUCAGUCACCAGCGAG | 11157 | ATGTTTTCAGTCACCAGCGAG |
| 5493 | AUUAACGCAGCCAACUUCAUC | 11158 | ATTAACGCAGCCAACTTCATC |
| 5494 | AUUAAGGGUGACCAGUGACUC | 11159 | ATTAAGGGTGACCAGTGACTC |
| 5495 | AUUACAGGCGUGAGCCACUGC | 11160 | ATTACAGGCGTGAGCCACTGC |
| 5496 | AUUACAGGCGUGAGGCACCUG | 11161 | ATTACAGGCGTGAGGCACCTG |
| 5497 | AUUCACGAUCACGGCUUUGAA | 11162 | ATTCACGATCACGGCTTTGAA |
| 5498 | AUUCAGCCAGAUCAUUUCCGA | 11163 | ATTCAGCCAGATCATTTCCGA |
| 5499 | AUUCAGGCCCCCUUUCUUGAU | 11164 | ATTCAGGCCCCCTTTCTTGAT |
| 5500 | AUUCAGGCUACAAAAACCUUC | 11165 | ATTCAGGCTACAAAAACCTTC |
| 5501 | AUUCAUUGACACGGGCUUUCC | 11166 | ATTCATTGACACGGGCTTTCC |
| 5502 | AUUCAUUUUUUAAGAGAUGGG | 11167 | ATTCATTTTTTAAGAGATGGG |
| 5503 | AUUCCCGGUCACACUGCCGGC | 11168 | ATTCCCGGTCACACTGCCGGC |
| 5504 | AUUCGCCGCUGUGACACUUGA | 11169 | ATTCGCCGCTGTGACACTTGA |
| 5505 | AUUCGGGGGUGACAGGGCAAA | 11170 | ATTCGGGGGTGACAGGGCAAA |
| 5506 | AUUCGUCAGGGCGACAGGUGG | 11171 | ATTCGTCAGGGCGACAGGTGG |
| 5507 | AUUCGUUGGUCCCGCACUCUU | 11172 | ATTCGTTGGTCCCGCACTCTT |
| 5508 | AUUCUAUUGCUGGCCACCUCC | 11173 | ATTCTATTGCTGGCCACCTCC |
| 5509 | AUUCUCCCAAAAAGCGUUGCA | 11174 | ATTCTCCCAAAAAGCGTTGCA |
| 5510 | AUUCUCCUGCCUCAGCCUCCC | 11175 | ATTCTCCTGCCTCAGCCTCCC |
| 5511 | AUUCUCUGGGACAGGUCAGAC | 11176 | ATTCTCTGGGACAGGTCAGAC |
| 5512 | AUUCUGAGGCAACGUUUCAAC | 11177 | ATTCTGAGGCAACGTTTCAAC |
| 5513 | AUUGACACGGGCUUUCCCUGG | 11178 | ATTGACACGGGCTTTCCCTGG |
| 5514 | AUUGAUCACAGUAAGACAUUC | 11179 | ATTGATCACAGTAAGACATTC |
| 5515 | AUUGCAGACGUGGGAACAGCC | 11180 | ATTGCAGACGTGGGAACAGCC |
| 5516 | AUUGCAUGGGCACUGUCCGAA | 11181 | ATTGCATGGGCACTGTCCGAA |
| 5517 | AUUGCCAAUCCCUUGUGACAU | 11182 | ATTGCCAATCCCTTGTGACAT |
| 5518 | AUUGCUCAGGGUGGUCCUCUC | 11183 | ATTGCTCAGGGTGGTCCTCTC |
| 5519 | AUUGCUGGCCACCUCCGUGUC | 11184 | ATTGCTGGCCACCTCCGTGTC |
| 5520 | AUUGGGCCACUGAAUGUUUUC | 11185 | ATTGGGCCACTGAATGTTTTC |
| 5521 | AUUGGGGAAGUGAAUGGCUUG | 11186 | ATTGGGGAAGTGAATGGCTTG |
| 5522 | AUUGGUGGAUGAGGCAGGGCC | 11187 | ATTGGTGGATGAGGCAGGGCC |
| 5523 | AUUGUCACUAUCUCCACCGUG | 11188 | ATTGTCACTATCTCCACCGTG |
| 5524 | AUUGUGUCACGGUGGAGAGAA | 11189 | ATTGTGTCACGGTGGAGAGAA |
| 5525 | AUUUAAGAAAUUUAAUUGAUC | 11190 | ATTTAAGAAATTTAATTGATC |
| 5526 | AUUUAAUUGAUCACAGUAAGA | 11191 | ATTTAATTGATCACAGTAAGA |
| 5527 | AUUUACGUGCUCCGAAACCAG | 11192 | ATTTACGTGCTCCGAAACCAG |
| 5528 | AUUUAUUUAUUUUUUUAUAGU | 11193 | ATTTATTTATTTTTTTATAGT |
| 5529 | AUUUAUUUUUUUAUAGUUGUA | 11194 | ATTTATTTTTTTATAGTTGTA |
| 5530 | AUUUCAGAAAGAACCUGAAGU | 11195 | ATTTCAGAAAGAACCTGAAGT |
| 5531 | AUUUCAGAGGCAAUAACCCCC | 11196 | ATTTCAGAGGCAATAACCCCC |
| 5532 | AUUUCCAGCCCCAGGGCCCCA | 11197 | ATTTCCAGCCCCAGGGCCCCA |
| 5533 | AUUUCCCGUCUUGGCACUGGA | 11198 | ATTTCCCGTCTTGGCACTGGA |
| 5534 | AUUUCCGACGCCAUGAAUCCC | 11199 | ATTTCCGACGCCATGAATCCC |
| 5535 | AUUUCCUCUGCCAGCAACGUC | 11200 | ATTTCCTCTGCCAGCAACGTC |
| 5536 | AUUUCUGAAAUAGGGUCUUCA | 11201 | ATTTCTGAAATAGGGTCTTCA |
| 5537 | AUUUGCAGGUGACAGACAAGC | 11202 | ATTTGCAGGTGACAGACAAGC |
| 5538 | AUUUGCAUAGAAGUGGAAGCA | 11203 | ATTTGCATAGAAGTGGAAGCA |
| 5539 | AUUUGGUGAAUGAGUUUGGUC | 11204 | ATTTGGTGAATGAGTTTGGTC |
| 5540 | AUUUGUCCUUGCAGUCGGGGC | 11205 | ATTTGTCCTTGCAGTCGGGGC |
| 5541 | AUUUUAUAUAUAUGUGUGUAU | 11206 | ATTTTATATATATGTGTGTAT |
| 5542 | AUUUUUAACUUUUUGGUUUUU | 11207 | ATTTTTAACTTTTTGGTTTTT |
| 5543 | AUUUUUAGUAGAGACGGGGUU | 11208 | ATTTTTAGTAGAGACGGGGTT |
| 5544 | AUUUUUUAAGAGAUGGGGUCU | 11209 | ATTTTTTAAGAGATGGGGTCT |
| 5545 | AUUUUUUGUAUUUUUAGUAGA | 11210 | ATTTTTTGTATTTTTAGTAGA |
| 5546 | AUUUUUUUAUAGUUGUAUUUC | 11211 | ATTTTTTTATAGTTGTATTTC |
| 5547 | CAAAAAAUCCCCACUAUCUGU | 11212 | CAAAAAATCCCCACTATCTGT |
| 5548 | CAAAAACCUUCUAUACAGUGG | 11213 | CAAAAACCTTCTATACAGTGG |
| 5549 | CAAAAAGCGUUGCACACAGUC | 11214 | CAAAAAGCGTTGCACACAGTC |
| 5550 | CAAAAAUAAAUAGAUUUUAUA | 11215 | CAAAAATAAATAGATTTTATA |
| 5551 | CAAAACUUCCUGGAGAGAAAU | 11216 | CAAAACTTCCTGGAGAGAAAT |
| 5552 | CAAAAUAAUAACACAAAUGCC | 11217 | CAAAATAATAACACAAATGCC |
| 5553 | CAAAAUACUUUGUCCUCAAAG | 11218 | CAAAATACTTTGTCCTCAAAG |
| 5554 | CAAAAUCCCAACCCAAGCCAU | 11219 | CAAAATCCCAACCCAAGCCAT |
| 5555 | CAAACAUAACAAAAAAUCCCC | 11220 | CAAACATAACAAAAAATCCCC |
| 5556 | CAAACCAUCCUCCCACCUCAG | 11221 | CAAACCATCCTCCCACCTCAG |
| 5557 | CAAACCCACCUCGAAAGACAC | 11222 | CAAACCCACCTCGAAAGACAC |
| 5558 | CAAACGAUCCAGACUGGAGGA | 11223 | CAAACGATCCAGACTGGAGGA |
| 5559 | CAAAGACGGCCAAGGAGAAGG | 11224 | CAAAGACGGCCAAGGAGAAGG |
| 5560 | CAAAGCUCUGGCAGGCAAUGC | 11225 | CAAAGCTCTGGCAGGCAATGC |
| 5561 | CAAAGGAAGACGAGGAGCACG | 11226 | CAAAGGAAGACGAGGAGCACG |
| 5562 | CAAAGGAAGACGAGGAGCACU | 11227 | CAAAGGAAGACGAGGAGCACT |
| 5563 | CAAAGGCUAACCUGGCUGUCU | 11228 | CAAAGGCTAACCTGGCTGTCT |
| 5564 | CAAAGUGCAAACAUAACAAAA | 11229 | CAAAGTGCAAACATAACAAAA |
| 5565 | CAAAGUGCUGGGAUUACAGGC | 11230 | CAAAGTGCTGGGATTACAGGC |
| 5566 | CAAAGUUGAUGCUGUUGAUGU | 11231 | CAAAGTTGATGCTGTTGATGT |
| 5567 | CAAAUAAUCUUUGUACAUAAA | 11232 | CAAATAATCTTTGTACATAAA |
| 5568 | CAAAUACAGCAACCAGGGUUU | 11233 | CAAATACAGCAACCAGGGTTT |
| 5569 | CAAAUGCCAAAUGUACACAGU | 11234 | CAAATGCCAAATGTACACAGT |
| 5570 | CAAAUGCCUGAGCUCAAACCA | 11235 | CAAATGCCTGAGCTCAAACCA |
| 5571 | CAAAUGUACACAGUGUACAAC | 11236 | CAAATGTACACAGTGTACAAC |
| 5572 | CAAAUGUGGACCUCAUCCUCU | 11237 | CAAATGTGGACCTCATCCTCT |
| 5573 | CAAAUUCAUUUUUUAAGAGAU | 11238 | CAAATTCATTTTTTAAGAGAT |
| 5574 | CAACAAGUUGACAUCGGAACC | 11239 | CAACAAGTTGACATCGGAACC |
| 5575 | CAACACACACGACAGAAAACA | 11240 | CAACACACACGACAGAAAACA |
| 5576 | CAACAGAGACAGUGCCCAGGA | 11241 | CAACAGAGACAGTGCCCAGGA |
| 5577 | CAACAGUAACACGGCGAUUUC | 11242 | CAACAGTAACACGGCGATTTC |
| 5578 | CAACCAAUAUACAAAGUGCAA | 11243 | CAACCAATATACAAAGTGCAA |
| 5579 | CAACCAGGGUUUGCAAAAAUA | 11244 | CAACCAGGGTTTGCAAAAATA |
| 5580 | CAACCCAAGCCAUUGCAUGGG | 11245 | CAACCCAAGCCATTGCATGGG |
| 5581 | CAACCCAGUAGAGGCGGCCAC | 11246 | CAACCCAGTAGAGGCGGCCAC |
| 5582 | CAACCUCCACCUCCUGGGCUC | 11247 | CAACCTCCACCTCCTGGGCTC |
| 5583 | CAACCUCGUAAGGAACCGAGG | 11248 | CAACCTCGTAAGGAACCGAGG |
| 5584 | CAACCUGCCCCUCUCUGUCCC | 11249 | CAACCTGCCCCTCTCTGTCCC |
| 5585 | CAACGACAAGAUUGGGGAAGU | 11250 | CAACGACAAGATTGGGGAAGT |
| 5586 | CAACGGGGACAUCAUUCUCCC | 11251 | CAACGGGGACATCATTCTCCC |
| 5587 | CAACGUCGCCCAGAGCUUGGU | 11252 | CAACGTCGCCCAGAGCTTGGT |
| 5588 | CAACGUUUCAACCUCGUAAGG | 11253 | CAACGTTTCAACCTCGTAAGG |
| 5589 | CAACUCCUGAACUCAAGUGAU | 11254 | CAACTCCTGAACTCAAGTGAT |
| 5590 | CAACUCUGAACUGAGAAAGUG | 11255 | CAACTCTGAACTGAGAAAGTG |
| 5591 | CAACUUCAUCGCUCAUGUCCU | 11256 | CAACTTCATCGCTCATGTCCT |
| 5592 | CAAGAUGGUCUUCCGGUUGCC | 11257 | CAAGATGGTCTTCCGGTTGCC |
| 5593 | CAAGAUUGGGGAAGUGAAUGG | 11258 | CAAGATTGGGGAAGTGAATGG |
| 5594 | CAAGCACGUCUCCUGGGACUC | 11259 | CAAGCACGTCTCCTGGGACTC |
| 5595 | CAAGCAGUCCCGGUCUGAGUC | 11260 | CAAGCAGTCCCGGTCTGAGTC |
| 5596 | CAAGCAUUCGUUGGUCCCGCA | 11261 | CAAGCATTCGTTGGTCCCGCA |
| 5597 | CAAGCCAUUGCAUGGGCACUG | 11262 | CAAGCCATTGCATGGGCACTG |
| 5598 | CAAGCUCCGCUUCCCGGGUUC | 11263 | CAAGCTCCGCTTCCCGGGTTC |
| 5599 | CAAGCUGGGUGCUGCAGAUCA | 11264 | CAAGCTGGGTGCTGCAGATCA |
| 5600 | CAAGGAGAAGGGGUGGGCCAG | 11265 | CAAGGAGAAGGGGTGGGCCAG |
| 5601 | CAAGGAGACCACGGGAAUGGA | 11266 | CAAGGAGACCACGGGAATGGA |
| 5602 | CAAGGAGGGGGGGGGAGAUUC | 11267 | CAAGGAGGGGGGGGGAGATTC |
| 5603 | CAAGGCCGGCGAGGUCUCAGG | 11268 | CAAGGCCGGCGAGGTCTCAGG |
| 5604 | CAAGGCGACGGUCCAGCGCAA | 11269 | CAAGGCGACGGTCCAGCGCAA |
| 5605 | CAAGGCUUGCAUAGAAGAGGU | 11270 | CAAGGCTTGCATAGAAGAGGT |
| 5606 | CAAGGUAACCGGGUGUCUCAG | 11271 | CAAGGTAACCGGGTGTCTCAG |
| 5607 | CAAGGUGGGGACGGAGGUGUC | 11272 | CAAGGTGGGGACGGAGGTGTC |
| 5608 | CAAGUAGCUGGGAUCACAGGC | 11273 | CAAGTAGCTGGGATCACAGGC |
| 5609 | CAAGUGAUCCAGCUCAGCCUC | 11274 | CAAGTGATCCAGCTCAGCCTC |
| 5610 | CAAGUUGACAUCGGAACCUGU | 11275 | CAAGTTGACATCGGAACCTGT |
| 5611 | CAAUAACCCCCUACACAGGGC | 11276 | CAATAACCCCCTACACAGGGC |
| 5612 | CAAUAUACAAAGUGCAAACAU | 11277 | CAATATACAAAGTGCAAACAT |
| 5613 | CAAUCCCUUGUGACAUCUUCA | 11278 | CAATCCCTTGTGACATCTTCA |
| 5614 | CAAUCUCGGCUCACUGCAAGC | 11279 | CAATCTCGGCTCACTGCAAGC |
| 5615 | CAAUGCUUUGGUCUUCUCUGU | 11280 | CAATGCTTTGGTCTTCTCTGT |
| 5616 | CAAUGGACAGAGCCCUCACGC | 11281 | CAATGGACAGAGCCCTCACGC |
| 5617 | CAAUGGCAGUUCUCGAUAUUC | 11282 | CAATGGCAGTTCTCGATATTC |
| 5618 | CAAUUGCCAAUCCCUUGUGAC | 11283 | CAATTGCCAATCCCTTGTGAC |
| 5619 | CAAUUUCCAGCCCCAGGGCCC | 11284 | CAATTTCCAGCCCCAGGGCCC |
| 5620 | CACAAAUGCCAAAUGUACACA | 11285 | CACAAATGCCAAATGTACACA |
| 5621 | CACACACGACAGAAAACAGUG | 11286 | CACACACGACAGAAAACAGTG |
| 5622 | CACACAGCUGCCCCCGAGACC | 11287 | CACACAGCTGCCCCCGAGACC |
| 5623 | CACACAGUCCAGUUCGUGCAA | 11288 | CACACAGTCCAGTTCGTGCAA |
| 5624 | CACACCAGUUCACUCCUCUUG | 11289 | CACACCAGTTCACTCCTCTTG |
| 5625 | CACACCAUUCAGGCCCCCUUU | 11290 | CACACCATTCAGGCCCCCTTT |
| 5626 | CACACCAUUCUCCUGCCUCAG | 11291 | CACACCATTCTCCTGCCTCAG |
| 5627 | CACACCUGGGUGCAGGCCACG | 11292 | CACACCTGGGTGCAGGCCACG |
| 5628 | CACACGACAGAAAACAGUGCA | 11293 | CACACGACAGAAAACAGTGCA |
| 5629 | CACACUGCCGGCUGCCAUGGA | 11294 | CACACTGCCGGCTGCCATGGA |
| 5630 | CACACUGGCACUUGUAGCCAC | 11295 | CACACTGGCACTTGTAGCCAC |
| 5631 | CACAGACGAACUGCCGAGAGA | 11296 | CACAGACGAACTGCCGAGAGA |
| 5632 | CACAGAGCACAGCGGAAACCG | 11297 | CACAGAGCACAGCGGAAACCG |
| 5633 | CACAGAUACUGGCAGCCGCCA | 11298 | CACAGATACTGGCAGCCGCCA |
| 5634 | CACAGCCAGCCCGUCGGGGGC | 11299 | CACAGCCAGCCCGTCGGGGGC |
| 5635 | CACAGCCUUGCAGGCCUUCGU | 11300 | CACAGCCTTGCAGGCCTTCGT |
| 5636 | CACAGCGCAGUUUUCCUCGUC | 11301 | CACAGCGCAGTTTTCCTCGTC |
| 5637 | CACAGCGCCAGCUGGAGUGGA | 11302 | CACAGCGCCAGCTGGAGTGGA |
| 5638 | CACAGCGGAAACCGGGGACUU | 11303 | CACAGCGGAAACCGGGGACTT |
| 5639 | CACAGCUGAAGUCCCCGGAUU | 11304 | CACAGCTGAAGTCCCCGGATT |
| 5640 | CACAGCUGCCCCCGAGACCAG | 11305 | CACAGCTGCCCCCGAGACCAG |
| 5641 | CACAGCUGGGGGAUGCAGGUG | 11306 | CACAGCTGGGGGATGCAGGTG |
| 5642 | CACAGGACAGCCUUGCUCGUC | 11307 | CACAGGACAGCCTTGCTCGTC |
| 5643 | CACAGGCACGUGCCACCGUAC | 11308 | CACAGGCACGTGCCACCGTAC |
| 5644 | CACAGGCACUCGUAGCCGAUC | 11309 | CACAGGCACTCGTAGCCGATC |
| 5645 | CACAGGGCCCCGAGAAUAGUC | 11310 | CACAGGGCCCCGAGAATAGTC |
| 5646 | CACAGGUGAGCACCGGGCAGG | 11311 | CACAGGTGAGCACCGGGCAGG |
| 5647 | CACAGUAAGACAUUCAGGCUA | 11312 | CACAGTAAGACATTCAGGCTA |
| 5648 | CACAGUCCAGUUCGUGCAAAU | 11313 | CACAGTCCAGTTCGTGCAAAT |
| 5649 | CACAGUGUACAACUCUGAACU | 11314 | CACAGTGTACAACTCTGAACT |
| 5650 | CACAGUGUUUUGUCCCUGGGG | 11315 | CACAGTGTTTTGTCCCTGGGG |
| 5651 | CACAUACCCAUCAACGACAAG | 11316 | CACATACCCATCAACGACAAG |
| 5652 | CACAUUAACGCAGCCAACUUC | 11317 | CACATTAACGCAGCCAACTTC |
| 5653 | CACCACGAUGGCCCUUGGCUU | 11318 | CACCACGATGGCCCTTGGCTT |
| 5654 | CACCACGCCCGGCUAAUUUUU | 11319 | CACCACGCCCGGCTAATTTTT |
| 5655 | CACCACGGAUUCAGCCAGAUC | 11320 | CACCACGGATTCAGCCAGATC |
| 5656 | CACCAGCGAGUAGAUGUCCAC | 11321 | CACCAGCGAGTAGATGTCCAC |
| 5657 | CACCAGCUGGAAGCCGUCGGG | 11322 | CACCAGCTGGAAGCCGTCGGG |
| 5658 | CACCAGGCUGGUGUCCUGACA | 11323 | CACCAGGCTGGTGTCCTGACA |
| 5659 | CACCAGUUCACUCCUCUUGGC | 11324 | CACCAGTTCACTCCTCTTGGC |
| 5660 | CACCAUCACAGCGCCAGCUGG | 11325 | CACCATCACAGCGCCAGCTGG |
| 5661 | CACCAUGCAUGGUUUUUUUUU | 11326 | CACCATGCATGGTTTTTTTTT |
| 5662 | CACCAUUCAGGCCCCCUUUCU | 11327 | CACCATTCAGGCCCCCTTTCT |
| 5663 | CACCAUUCUCCUGCCUCAGCC | 11328 | CACCATTCTCCTGCCTCAGCC |
| 5664 | CACCCAUCUCCCAGAAGCCAC | 11329 | CACCCATCTCCCAGAAGCCAC |
| 5665 | CACCCGCCUCGGCCUCCCAAA | 11330 | CACCCGCCTCGGCCTCCCAAA |
| 5666 | CACCCUCCAGGUUCACGCAGA | 11331 | CACCCTCCAGGTTCACGCAGA |
| 5667 | CACCGGGCAGGAGGCCUCGUC | 11332 | CACCGGGCAGGAGGCCTCGTC |
| 5668 | CACCGUACCCAGCUGAUUUUU | 11333 | CACCGTACCCAGCTGATTTTT |
| 5669 | CACCGUGCAUGUUUUAAACAC | 11334 | CACCGTGCATGTTTTAAACAC |
| 5670 | CACCGUGGUGAGCCCAGGGGU | 11335 | CACCGTGGTGAGCCCAGGGGT |
| 5671 | CACCUCAGCCUCGCCAUGCCU | 11336 | CACCTCAGCCTCGCCATGCCT |
| 5672 | CACCUCCAGAACUGAGGAAUG | 11337 | CACCTCCAGAACTGAGGAATG |
| 5673 | CACCUCCGUGUCCAGAGCGAC | 11338 | CACCTCCGTGTCCAGAGCGAC |
| 5674 | CACCUCCUGGGCUCAGGCGAU | 11339 | CACCTCCTGGGCTCAGGCGAT |
| 5675 | CACCUCGAAAGACACUGGGUG | 11340 | CACCTCGAAAGACACTGGGTG |
| 5676 | CACCUGGGUGCAGGCCACGUG | 11341 | CACCTGGGTGCAGGCCACGTG |
| 5677 | CACCUGUGUCUGGCCAAAUUC | 11342 | CACCTGTGTCTGGCCAAATTC |
| 5678 | CACGACAGAAAACAGUGCAAA | 11343 | CACGACAGAAAACAGTGCAAA |
| 5679 | CACGACAGCCUCAAAUGCCUG | 11344 | CACGACAGCCTCAAATGCCTG |
| 5680 | CACGACCUGCUGUGUCCUAGC | 11345 | CACGACCTGCTGTGTCCTAGC |
| 5681 | CACGAGGAAAGGAAGAAACCA | 11346 | CACGAGGAAAGGAAGAAACCA |
| 5682 | CACGAUCACGGCUUUGAAGUC | 11347 | CACGATCACGGCTTTGAAGTC |
| 5683 | CACGAUGGCCCUUGGCUUGGA | 11348 | CACGATGGCCCTTGGCTTGGA |
| 5684 | CACGAUGGGGAGGACAAUGGA | 11349 | CACGATGGGGAGGACAATGGA |
| 5685 | CACGCAGAGCUGGCUGCAGGU | 11350 | CACGCAGAGCTGGCTGCAGGT |
| 5686 | CACGCCACGUCAUCCUCCAGA | 11351 | CACGCCACGTCATCCTCCAGA |
| 5687 | CACGCCCGGCUAAUUUUUUGU | 11352 | CACGCCCGGCTAATTTTTTGT |
| 5688 | CACGCCCUUGGUAUCCGCAAC | 11353 | CACGCCCTTGGTATCCGCAAC |
| 5689 | CACGCGGGAGUCUGACGGAGG | 11354 | CACGCGGGAGTCTGACGGAGG |
| 5690 | CACGCUACUGGGCUUCUUCUC | 11355 | CACGCTACTGGGCTTCTTCTC |
| 5691 | CACGCUCUGUCACACAGCUGC | 11356 | CACGCTCTGTCACACAGCTGC |
| 5692 | CACGGAUUCAGCCAGAUCAUU | 11357 | CACGGATTCAGCCAGATCATT |
| 5693 | CACGGCCAAGGUAACCGGGUG | 11358 | CACGGCCAAGGTAACCGGGTG |
| 5694 | CACGGCCCCCACAGCUGAAGU | 11359 | CACGGCCCCCACAGCTGAAGT |
| 5695 | CACGGCGAUUUCAGAAAGAAC | 11360 | CACGGCGATTTCAGAAAGAAC |
| 5696 | CACGGCUUUGAAGUCCCGGUC | 11361 | CACGGCTTTGAAGTCCCGGTC |
| 5697 | CACGGGAAUGGAAGUGGGUAG | 11362 | CACGGGAATGGAAGTGGGTAG |
| 5698 | CACGGGCUUUCCCUGGCCUGG | 11363 | CACGGGCTTTCCCTGGCCTGG |
| 5699 | CACGGUGGAGAGAAACUCAAA | 11364 | CACGGTGGAGAGAAACTCAAA |
| 5700 | CACGGUUCCCUGCACUGGGGG | 11365 | CACGGTTCCCTGCACTGGGGG |
| 5701 | CACGUAAAGACCCCUACAGCG | 11366 | CACGTAAAGACCCCTACAGCG |
| 5702 | CACGUCAUCCUCCAGACUGAC | 11367 | CACGTCATCCTCCAGACTGAC |
| 5703 | CACGUCUCCUGGGACUCAUCA | 11368 | CACGTCTCCTGGGACTCATCA |
| 5704 | CACGUCUUGGGGGAGCACGUC | 11369 | CACGTCTTGGGGGAGCACGTC |
| 5705 | CACGUCUUGGGGGGACAGCCU | 11370 | CACGTCTTGGGGGGACAGCCT |
| 5706 | CACGUGCCACCGUACCCAGCU | 11371 | CACGTGCCACCGTACCCAGCT |
| 5707 | CACGUGGCUGAAGAUCUCGGC | 11372 | CACGTGGCTGAAGATCTCGGC |
| 5708 | CACGUGUCCUCACGGCCAAGG | 11373 | CACGTGTCCTCACGGCCAAGG |
| 5709 | CACGUGUUAGCCAGGAUGGUC | 11374 | CACGTGTTAGCCAGGATGGTC |
| 5710 | CACGUUCCUCAGGUUGGGGAU | 11375 | CACGTTCCTCAGGTTGGGGAT |
| 5711 | CACGUUGACUCCUGAGUUUAG | 11376 | CACGTTGACTCCTGAGTTTAG |
| 5712 | CACUAGGUGGGGGACAUAAA | 11377 | CACTAGGTGGGCGGACATAAA |
| 5713 | CACUAUCUCCACCGUGGUGAG | 11378 | CACTATCTCCACCGTGGTGAG |
| 5714 | CACUAUCUGUACAGGGACGCA | 11379 | CACTATCTGTACAGGGACGCA |
| 5715 | CACUCAGCGCUGCCAUCGCAG | 11380 | CACTCAGCGCTGCCATCGCAG |
| 5716 | CACUCAUACAUACAACGGGGA | 11381 | CACTCATACATACAACGGGGA |
| 5717 | CACUCAUCCGAGCCAUCUUCG | 11382 | CACTCATCCGAGCCATCTTCG |
| 5718 | CACUCAUCGAUAUCGCACUCU | 11383 | CACTCATCGATATCGCACTCT |
| 5719 | CACUCAUCGAUAUCUUCGCAU | 11384 | CACTCATCGATATCTTCGCAT |
| 5720 | CACUCCUCUUGGCUGGGUGAG | 11385 | CACTCCTCTTGGCTGGGTGAG |
| 5721 | CACUCGCCACUUAGGCAGUGG | 11386 | CACTCGCCACTTAGGCAGTGG |
| 5722 | CACUCGUAGCCGAUCUUAAGG | 11387 | CACTCGTAGCCGATCTTAAGG |
| 5723 | CACUCUUUGAUGGGUUCAUCU | 11388 | CACTCTTTGATGGGTTCATCT |
| 5724 | CACUGAAAAUGGCUUCGUUGA | 11389 | CACTGAAAATGGCTTCGTTGA |
| 5725 | CACUGAACAAAUACAGCAACC | 11390 | CACTGAACAAATACAGCAACC |
| 5726 | CACUGAAUGUUUUCAGUCACC | 11391 | CACTGAATGTTTTCAGTCACC |
| 5727 | CACUGAGGAGAUCUAGGGUGA | 11392 | CACTGAGGAGATCTAGGGTGA |
| 5728 | CACUGCAACCUCCACCUCCUG | 11393 | CACTGCAACCTCCACCTCCTG |
| 5729 | CACUGCAAGCUCCGCUUCCCG | 11394 | CACTGCAAGCTCCGCTTCCCG |
| 5730 | CACUGCAGCCUCAGCCUCUCU | 11395 | CACTGCAGCCTCAGCCTCTCT |
| 5731 | CACUGCAGCCUCAGCCUCUGU | 11396 | CACTGCAGCCTCAGCCTCTGT |
| 5732 | CACUGCAGUCCCCGCCGCGGC | 11397 | CACTGCAGTCCCCGCCGCGGC |
| 5733 | CACUGCCGGCUGCCAUGGAUG | 11398 | CACTGCCGGCTGCCATGGATG |
| 5734 | CACUGCGCCCGGCCUCACCGU | 11399 | CACTGCGCCCGGCCTCACCGT |
| 5735 | CACUGGAACUCGUUUCUUUCG | 11400 | CACTGGAACTCGTTTCTTTCG |
| 5736 | CACUGGAAGCUGGCGGGACCA | 11401 | CACTGGAAGCTGGCGGGACCA |
| 5737 | CACUGGAAUUCGUCAGGGCGA | 11402 | CACTGGAATTCGTCAGGGCGA |
| 5738 | CACUGGCACUUGUAGCCACCC | 11403 | CACTGGCACTTGTAGCCACCC |
| 5739 | CACUGGGGGGGACACAGUGUU | 11404 | CACTGGGGGGGACACAGTGTT |
| 5740 | CACUGGGUGGGUCAUGAUUCG | 11405 | CACTGGGTGGGTCATGATTCG |
| 5741 | CACUGUAGGGAGAGGCACGCU | 11406 | CACTGTAGGGAGAGGCACGCT |
| 5742 | CACUGUCCGAAGCCUGUUCUG | 11407 | CACTGTCCGAAGCCTGTTCTG |
| 5743 | CACUGUCUCUCCGGACAUCAG | 11408 | CACTGTCTCTCCGGACATCAG |
| 5744 | CACUUAAUAAAUAUUAAGGGU | 11409 | CACTTAATAAATATTAAGGGT |
| 5745 | CACUUAGGCAGUGGAACUCGA | 11410 | CACTTAGGCAGTGGAACTCGA |
| 5746 | CACUUCCCAUCGUGGCAGCGA | 11411 | CACTTCCCATCGTGGCAGCGA |
| 5747 | CACUUGAACUUGUUGGGUCCC | 11412 | CACTTGAACTTGTTGGGTCCC |
| 5748 | CACUUGCUUCUCUGGGCCUGA | 11413 | CACTTGCTTCTCTGGGCCTGA |
| 5749 | CACUUGGCCAUCGCACCUCCA | 11414 | CACTTGGCCATCGCACCTCCA |
| 5750 | CACUUGUAGCCACCCUCCAGG | 11415 | CACTTGTAGCCACCCTCCAGG |
| 5751 | CACUUGUAGGAGAUGCAUUUC | 11416 | CACTTGTAGGAGATGCATTTC |
| 5752 | CAGAAAACAGUGCAAAAUAAU | 11417 | CAGAAAACAGTGCAAAATAAT |
| 5753 | CAGAAAGAACCUGAAGUCCCG | 11418 | CAGAAAGAACCTGAAGTCCCG |
| 5754 | CAGAAAGGCUUAUCACGGUUC | 11419 | CAGAAAGGCTTATCACGGTTC |
| 5755 | CAGAACUGAGGAAUGCAGCGG | 11420 | CAGAACTGAGGAATGCAGCGG |
| 5756 | CAGAAGCCACUCAUACAUACA | 11421 | CAGAAGCCACTCATACATACA |
| 5757 | CAGACAAGCACGUCUCCUGGG | 11422 | CAGACAAGCACGTCTCCTGGG |
| 5758 | CAGACAGCCUUGCUCGUCUGA | 11423 | CAGACAGCCTTGCTCGTCTGA |
| 5759 | CAGACCAAGGAGGGGGGGGGA | 11424 | CAGACCAAGGAGGGGCGGGGA |
| 5760 | CAGACCAGUAGAUUCUAUUGC | 11425 | CAGACCAGTAGATTCTATTGC |
| 5761 | CAGACCCACUUGUAGGAGAUG | 11426 | CAGACCCACTTGTAGGAGATG |
| 5762 | CAGACGAACUGCCGAGAGAUG | 11427 | CAGACGAACTGCCGAGAGATG |
| 5763 | CAGACGUGGGAACAGCCGCCG | 11428 | CAGACGTGGGAACAGCCGCCG |
| 5764 | CAGACUGACCAUCGAGGGGUA | 11429 | CAGACTGACCATCGAGGGGTA |
| 5765 | CAGACUGACCAUCUGUCUCGA | 11430 | CAGACTGACCATCTGTCTCGA |
| 5766 | CAGACUGCGGACUGCAGUGGC | 11431 | CAGACTGCGGACTGCAGTGGC |
| 5767 | CAGACUGGAGGAUUUAUUUAU | 11432 | CAGACTGGAGGATTTATTTAT |
| 5768 | CAGACUUUGUCCAGGGUGAUG | 11433 | CAGACTTTGTCCAGGGTGATG |
| 5769 | CAGAGACAGUGCCCAGGACAG | 11434 | CAGAGACAGTGCCCAGGACAG |
| 5770 | CAGAGACCCAGCUCCGUGGCG | 11435 | CAGAGACCCAGCTCCGTGGCG |
| 5771 | CAGAGCACAGCGGAAACCGGG | 11436 | CAGAGCACAGCGGAAACCGGG |
| 5772 | CAGAGCACUGGAAUUCGUCAG | 11437 | CAGAGCACTGGAATTCGTCAG |
| 5773 | CAGAGCCAUCCUGGCACUCAG | 11438 | CAGAGCCATCCTGGCACTCAG |
| 5774 | CAGAGCCCUCACGCUACUGGG | 11439 | CAGAGCCCTCACGCTACTGGG |
| 5775 | CAGAGCGACCACGUUCCUCAG | 11440 | CAGAGCGACCACGTTCCTCAG |
| 5776 | CAGAGCUGGCUGCAGGUGUCG | 11441 | CAGAGCTGGCTGCAGGTGTCG |
| 5777 | CAGAGCUUGGUGAGACAUUGU | 11442 | CAGAGCTTGGTGAGACATTGT |
| 5778 | CAGAGGCAAUAACCCCCUACA | 11443 | CAGAGGCAATAACCCCCTACA |
| 5779 | CAGAGGCCAUGGGCUGCUGAU | 11444 | CAGAGGCCATGGGCTGCTGAT |
| 5780 | CAGAGUCGGUCCAGUAGAUGU | 11445 | CAGAGTCGGTCCAGTAGATGT |
| 5781 | CAGAGUGUCACAUUAACGCAG | 11446 | CAGAGTGTCACATTAACGCAG |
| 5782 | CAGAGUGUCAGACAGCCUUGC | 11447 | CAGAGTGTCAGACAGCCTTGC |
| 5783 | CAGAUACUGGCAGCCGCCAUU | 11448 | CAGATACTGGCAGCCGCCATT |
| 5784 | CAGAUCAUUCUCUGGGACAGG | 11449 | CAGATCATTCTCTGGGACAGG |
| 5785 | CAGAUCAUUUCCGACGCCAUG | 11450 | CAGATCATTTCCGACGCCATG |
| 5786 | CAGAUGAAUAAAUAUAUAAAA | 11451 | CAGATGAATAAATATATAAAA |
| 5787 | CAGAUGUUCACGCCACGUCAU | 11452 | CAGATGTTCACGCCACGTCAT |
| 5788 | CAGAUUUGUCCUUGCAGUCGG | 11453 | CAGATTTGTCCTTGCAGTCGG |
| 5789 | CAGCAACCAGGGUUUGCAAAA | 11454 | CAGCAACCAGGGTTTGCAAAA |
| 5790 | CAGCAACGUCGCCCAGAGCUU | 11455 | CAGCAACGTCGCCCAGAGCTT |
| 5791 | CAGCAGCAUGCCGUCCGGGCA | 11456 | CAGCAGCATGCCGTCCGGGCA |
| 5792 | CAGCAUGCCGUCCGGGCAGGC | 11457 | CAGCATGCCGTCCGGGCAGGC |
| 5793 | CAGCCAACAAGUUGACAUCGG | 11458 | CAGCCAACAAGTTGACATCGG |
| 5794 | CAGCCAACUUCAUCGCUCAUG | 11459 | CAGCCAACTTCATCGCTCATG |
| 5795 | CAGCCACACCUGGGUGCAGGC | 11460 | CAGCCACACCTGGGTGCAGGC |
| 5796 | CAGCCAGAUCAUUUCCGACGC | 11461 | CAGCCAGATCATTTCCGACGC |
| 5797 | CAGCCAGCCCGUCGGGGGCCU | 11462 | CAGCCAGCCCGTCGGGGGCCT |
| 5798 | CAGCCCCAGGGCCCCAUGCUC | 11463 | CAGCCCCAGGGCCCCATGCTC |
| 5799 | CAGCCCGUCGGGGGCCUGGAU | 11464 | CAGCCCGTCGGGGGCCTGGAT |
| 5800 | CAGCCGCCAUUGCUCAGGGUG | 11465 | CAGCCGCCATTGCTCAGGGTG |
| 5801 | CAGCCGCCGUUGUUGUCCAAG | 11466 | CAGCCGCCGTTGTTGTCCAAG |
| 5802 | CAGCCGGGAGGUGUCGGGAAC | 11467 | CAGCCGGGAGGTGTCGGGAAC |
| 5803 | CAGCCUCAAAUGCCUGAGCUC | 11468 | CAGCCTCAAATGCCTGAGCTC |
| 5804 | CAGCCUCAGCCUCUCUUGGCU | 11469 | CAGCCTCAGCCTCTCTTGGCT |
| 5805 | CAGCCUCAGCCUCUGUGAGGC | 11470 | CAGCCTCAGCCTCTGTGAGGC |
| 5806 | CAGCCUCCCAAAGUGCUGGGA | 11471 | CAGCCTCCCAAAGTGCTGGGA |
| 5807 | CAGCCUCCCAAGUAGCUGGGA | 11472 | CAGCCTCCCAAGTAGCTGGGA |
| 5808 | CAGCCUCCCGAGUAGCUGGGA | 11473 | CAGCCTCCCGAGTAGCTGGGA |
| 5809 | CAGCCUCGCCAUGCCUGGCCA | 11474 | CAGCCTCGCCATGCCTGGCCA |
| 5810 | CAGCCUCUCUUGGCUGGGUGA | 11475 | CAGCCTCTCTTGGCTGGGTGA |
| 5811 | CAGCCUCUGCCAGGCAGUGUC | 11476 | CAGCCTCTGCCAGGCAGTGTC |
| 5812 | CAGCCUCUGUGAGGCAGCUCC | 11477 | CAGCCTCTGTGAGGCAGCTCC |
| 5813 | CAGCCUCUUUUCAUCCUCCAA | 11478 | CAGCCTCTTTTCATCCTCCAA |
| 5814 | CAGCCUUGCAGGCCUUCGUGU | 11479 | CAGCCTTGCAGGCCTTCGTGT |
| 5815 | CAGCCUUGCUCGUCUGAGCCG | 11480 | CAGCCTTGCTCGTCTGAGCCG |
| 5816 | CAGCGAAACUCGUCCUGGGAG | 11481 | CAGCGAAACTCGTCCTGGGAG |
| 5817 | CAGCGAGUAGAUGUCCACACC | 11482 | CAGCGAGTAGATGTCCACACC |
| 5818 | CAGCGCAAUUUCCAGCCCCAG | 11483 | CAGCGCAATTTCCAGCCCCAG |
| 5819 | CAGCGCAGUUUUCCUCGUCAG | 11484 | CAGCGCAGTTTTCCTCGTCAG |
| 5820 | CAGCGCCAGCUGGAGUGGAUG | 11485 | CAGCGCCAGCTGGAGTGGATG |
| 5821 | CAGCGCUGCCAUCGCAGACCC | 11486 | CAGCGCTGCCATCGCAGACCC |
| 5822 | CAGCGCUGCGGCCACUCAUCC | 11487 | CAGCGCTGCGGCCACTCATCC |
| 5823 | CAGCGGAAACCGGGGACUUCG | 11488 | CAGCGGAAACCGGGGACTTCG |
| 5824 | CAGCGGUUGACACGGCCCCCA | 11489 | CAGCGGTTGACACGGCCCCCA |
| 5825 | CAGCGUCAUCUUCCUGACCUC | 11490 | CAGCGTCATCTTCCTGACCTC |
| 5826 | CAGCUCAGCCUCCCAAAGUGC | 11491 | CAGCTCAGCCTCCCAAAGTGC |
| 5827 | CAGCUCCGUGGCGAUGGGGAG | 11492 | CAGCTCCGTGGCGATGGGGAG |
| 5828 | CAGCUCCUCAUGUCCCUGGCC | 11493 | CAGCTCCTCATGTCCCTGGCC |
| 5829 | CAGCUGAAGUCCCCGGAUUUG | 11494 | CAGCTGAAGTCCCCGGATTTG |
| 5830 | CAGCUGAUUUUUAACUUUUUG | 11495 | CAGCTGATTTTTAACTTTTTG |
| 5831 | CAGCUGCCCCCGAGACCAGAG | 11496 | CAGCTGCCCCCGAGACCAGAG |
| 5832 | CAGCUGGAAGCCGUCGGGGCA | 11497 | CAGCTGGAAGCCGTCGGGGCA |
| 5833 | CAGCUGGAAGCCUUCCUCACA | 11498 | CAGCTGGAAGCCTTCCTCACA |
| 5834 | CAGCUGGAGUGGAUGCACUCG | 11499 | CAGCTGGAGTGGATGCACTCG |
| 5835 | CAGCUGGGGGAUGCAGGUGGA | 11500 | CAGCTGGGGGATGCAGGTGGA |
| 5836 | CAGGAACCGCGGAGGAACGCC | 11501 | CAGGAACCGCGGAGGAACGCC |
| 5837 | CAGGAAGGGUUCUGGGCAGGG | 11502 | CAGGAAGGGTTCTGGGCAGGG |
| 5838 | CAGGACAAGGUGGGGACGGAG | 11503 | CAGGACAAGGTGGGGACGGAG |
| 5839 | CAGGACAGAGUCGGUCCAGUA | 11504 | CAGGACAGAGTCGGTCCAGTA |
| 5840 | CAGGACAGCCUUGCUCGUCUG | 11505 | CAGGACAGCCTTGCTCGTCTG |
| 5841 | CAGGAGGCCUCGUCUGAGCCG | 11506 | CAGGAGGCCTCGTCTGAGCCG |
| 5842 | CAGGAGGGCCCAGGAUAACGG | 11507 | CAGGAGGGCCCAGGATAACGG |
| 5843 | CAGGAUAACGGAGGGCGACUC | 11508 | CAGGATAACGGAGGGCGACTC |
| 5844 | CAGGAUCCACCACGAUGGCCC | 11509 | CAGGATCCACCACGATGGCCC |
| 5845 | CAGGAUGGUCUCGAUCUCCUG | 11510 | CAGGATGGTCTCGATCTCCTG |
| 5846 | CAGGCAAAGGAAGACGAGGAG | 11511 | CAGGCAAAGGAAGACGAGGAG |
| 5847 | CAGGCAAUGCUUUGGUCUUCU | 11512 | CAGGCAATGCTTTGGTCTTCT |
| 5848 | CAGGCACGUGCCACCGUACCC | 11513 | CAGGCACGTGCCACCGTACCC |
| 5849 | CAGGCACUCGUAGCCGAUCUU | 11514 | CAGGCACTCGTAGCCGATCTT |
| 5850 | CAGGCACUUAAUAAAUAUUAA | 11515 | CAGGCACTTAATAAATATTAA |
| 5851 | CAGGCAGAUGUUCACGCCACG | 11516 | CAGGCAGATGTTCACGCCACG |
| 5852 | CAGGCAGCCGGGAGGUGUCGG | 11517 | CAGGCAGCCGGGAGGTGTCGG |
| 5853 | CAGGCAGUGUCCCGACCCGGA | 11518 | CAGGCAGTGTCCCGACCCGGA |
| 5854 | CAGGCCACGUGUCCUCACGGC | 11519 | CAGGCCACGTGTCCTCACGGC |
| 5855 | CAGGCCAGACUGCGGACUGCA | 11520 | CAGGCCAGACTGCGGACTGCA |
| 5856 | CAGGCCCACAGCUGGGGGAUG | 11521 | CAGGCCCACAGCTGGGGGATG |
| 5857 | CAGGCCCCCUUUCUUGAUCUU | 11522 | CAGGCCCCCTTTCTTGATCTT |
| 5858 | CAGGCCUGGUCUCCAACUCCU | 11523 | CAGGCCTGGTCTCCAACTCCT |
| 5859 | CAGGCCUUCGUGUGGGGGUCC | 11524 | CAGGCCTTCGTGTGGGGGTCC |
| 5860 | CAGGCGAUCCUCCUGCCUCAG | 11525 | CAGGCGATCCTCCTGCCTCAG |
| 5861 | CAGGCGCAGGUAAACUUGGGC | 11526 | CAGGCGCAGGTAAACTTGGGC |
| 5862 | CAGGCGUGAGCCACUGCGCCC | 11527 | CAGGCGTGAGCCACTGCGCCC |
| 5863 | CAGGCGUGAGGCACCUGUGUC | 11528 | CAGGCGTGAGGCACCTGTGTC |
| 5864 | CAGGCUACAAAAACCUUCUAU | 11529 | CAGGCTACAAAAACCTTCTAT |
| 5865 | CAGGCUCUGCCUCUGGGAAGU | 11530 | CAGGCTCTGCCTCTGGGAAGT |
| 5866 | CAGGCUGGAAAGCAGUGGCAU | 11531 | CAGGCTGGAAAGCAGTGGCAT |
| 5867 | CAGGCUGGAGUGCAGUGGCUC | 11532 | CAGGCTGGAGTGCAGTGGCTC |
| 5868 | CAGGCUGGUGUCCUGACAGCC | 11533 | CAGGCTGGTGTCCTGACAGCC |
| 5869 | CAGGGACGCAUUUACGUGCUC | 11534 | CAGGGACGCATTTACGTGCTC |
| 5870 | CAGGGACGGGACUCCAGGCAG | 11535 | CAGGGACGGGACTCCAGGCAG |
| 5871 | CAGGGAGGCACAGAUACUGGC | 11536 | CAGGGAGGCACAGATACTGGC |
| 5872 | CAGGGCAAAGGCUAACCUGGC | 11537 | CAGGGCAAAGGCTAACCTGGC |
| 5873 | CAGGGCCAGGGGGUCCCGUUG | 11538 | CAGGGCCAGGGGGTCCCGTTG |
| 5874 | CAGGGCCCCAUGCUCGCAGCC | 11539 | CAGGGCCCCATGCTCGCAGCC |
| 5875 | CAGGGCCCCGAGAAUAGUCAC | 11540 | CAGGGCCCCGAGAATAGTCAC |
| 5876 | CAGGGCGACAGGUGGCCACAG | 11541 | CAGGGCGACAGGTGGCCACAG |
| 5877 | CAGGGGCUACUGUCCCCUUGG | 11542 | CAGGGGCTACTGTCCCCTTGG |
| 5878 | CAGGGGGUCCCGUUGGUGCCA | 11543 | CAGGGGGTCCCGTTGGTGCCA |
| 5879 | CAGGGGUGGCCCCAGGCAGCC | 11544 | CAGGGGTGGCCCCAGGCAGCC |
| 5880 | CAGGGUGAUGCAUUCGCCGCU | 11545 | CAGGGTGATGCATTCGCCGCT |
| 5881 | CAGGGUGGUCCUCUCACACCA | 11546 | CAGGGTGGTCCTCTCACACCA |
| 5882 | CAGGGUUUGCAAAAAUAAAUA | 11547 | CAGGGTTTGCAAAAATAAATA |
| 5883 | CAGGUAAACUUGGGCGAGUGG | 11548 | CAGGTAAACTTGGGCGAGTGG |
| 5884 | CAGGUCAGACCAGUAGAUUCU | 11549 | CAGGTCAGACCAGTAGATTCT |
| 5885 | CAGGUCGGGUGGUUGUGUGCU | 11550 | CAGGTCGGGTGGTTGTGTGCT |
| 5886 | CAGGUGACAGACAAGCACGUC | 11551 | CAGGTGACAGACAAGCACGTC |
| 5887 | CAGGUGAGCACCGGGCAGGAG | 11552 | CAGGTGAGCACCGGGCAGGAG |
| 5888 | CAGGUGCCCGCCACCACGCCC | 11553 | CAGGTGCCCGCCACCACGCCC |
| 5889 | CAGGUGGAGCUGUUGCACUGG | 11554 | CAGGTGGAGCTGTTGCACTGG |
| 5890 | CAGGUGGCCACAGCGCAGUUU | 11555 | CAGGTGGCCACAGCGCAGTTT |
| 5891 | CAGGUGGCCACAGGACAGCCU | 11556 | CAGGTGGCCACAGGACAGCCT |
| 5892 | CAGGUGUCGGGAUCCUGACAC | 11557 | CAGGTGTCGGGATCCTGACAC |
| 5893 | CAGGUUCACGCAGAGCUGGCU | 11558 | CAGGTTCACGCAGAGCTGGCT |
| 5894 | CAGGUUGGGGAUGAGGCUGGU | 11559 | CAGGTTGGGGATGAGGCTGGT |
| 5895 | CAGUAACACGGCGAUUUCAGA | 11560 | CAGTAACACGGCGATTTCAGA |
| 5896 | CAGUAAGACAUUCAGGCUACA | 11561 | CAGTAAGACATTCAGGCTACA |
| 5897 | CAGUACAUGAAGCCAUGAACA | 11562 | CAGTACATGAAGCCATGAACA |
| 5898 | CAGUAGAGGCGGCCACUGAGG | 11563 | CAGTAGAGGCGGCCACTGAGG |
| 5899 | CAGUAGAUGUUGCUGUGGAUC | 11564 | CAGTAGATGTTGCTGTGGATC |
| 5900 | CAGUAGAUUCUAUUGCUGGCC | 11565 | CAGTAGATTCTATTGCTGGCC |
| 5901 | CAGUAGGUUUUCAGCCAACAA | 11566 | CAGTAGGTTTTCAGCCAACAA |
| 5902 | CAGUCACCAGCGAGUAGAUGU | 11567 | CAGTCACCAGCGAGTAGATGT |
| 5903 | CAGUCAGUCCAGUACAUGAAG | 11568 | CAGTCAGTCCAGTACATGAAG |
| 5904 | CAGUCAUAUUCCCGGUCACAC | 11569 | CAGTCATATTCCCGGTCACAC |
| 5905 | CAGUCCACAGCCAGCCCGUCG | 11570 | CAGTCCACAGCCAGCCCGTCG |
| 5906 | CAGUCCACUUGGCCAUCGCAC | 11571 | CAGTCCACTTGGCCATCGCAC |
| 5907 | CAGUCCAGUACAUGAAGCCAU | 11572 | CAGTCCAGTACATGAAGCCAT |
| 5908 | CAGUCCAGUUCGUGCAAAUAA | 11573 | CAGTCCAGTTCGTGCAAATAA |
| 5909 | CAGUCCCCGCCGCGGCGAGGA | 11574 | CAGTCCCCGCCGCGGCGAGGA |
| 5910 | CAGUCCCGGCAGUCUCUAGCC | 11575 | CAGTCCCGGCAGTCTCTAGCC |
| 5911 | CAGUCCCGGUCUGAGUCACAG | 11576 | CAGTCCCGGTCTGAGTCACAG |
| 5912 | CAGUCGGGGCCACCAUCACAG | 11577 | CAGTCGGGGCCACCATCACAG |
| 5913 | CAGUCGGGGUCGUUGUCGCAG | 11578 | CAGTCGGGGTCGTTGTCGCAG |
| 5914 | CAGUCUCUAGCCAUGUUGCAG | 11579 | CAGTCTCTAGCCATGTTGCAG |
| 5915 | CAGUGACUCAGGCUCUGCCUC | 11580 | CAGTGACTCAGGCTCTGCCTC |
| 5916 | CAGUGCAAAAUAAUAACACAA | 11581 | CAGTGCAAAATAATAACACAA |
| 5917 | CAGUGCAACAGUAACACGGCG | 11582 | CAGTGCAACAGTAACACGGCG |
| 5918 | CAGUGCCCAGGACAGAGUCGG | 11583 | CAGTGCCCAGGACAGAGTCGG |
| 5919 | CAGUGGAACUCGAAGGCCGAG | 11584 | CAGTGGAACTCGAAGGCCGAG |
| 5920 | CAGUGGCAUAAUCAUAGCUCA | 11585 | CAGTGGCATAATCATAGCTCA |
| 5921 | CAGUGGCGCAAUCUCGGCUCA | 11586 | CAGTGGCGCAATCTCGGCTCA |
| 5922 | CAGUGGCUCGAUCAUGGCUCA | 11587 | CAGTGGCTCGATCATGGCTCA |
| 5923 | CAGUGGUUUAAAAAGUGACAC | 11588 | CAGTGGTTTAAAAAGTGACAC |
| 5924 | CAGUGUACAACUCUGAACUGA | 11589 | CAGTGTACAACTCTGAACTGA |
| 5925 | CAGUGUCCCGACCCGGAUCAC | 11590 | CAGTGTCCCGACCCGGATCAC |
| 5926 | CAGUGUUUUGUCCCUGGGGAC | 11591 | CAGTGTTTTGTCCCTGGGGAC |
| 5927 | CAGUUCACUCCUCUUGGCUGG | 11592 | CAGTTCACTCCTCTTGGCTGG |
| 5928 | CAGUUCGUGCAAAUAAUCUUU | 11593 | CAGTTCGTGCAAATAATCTTT |
| 5929 | CAGUUCUCGAUAUUCACGAUC | 11594 | CAGTTCTCGATATTCACGATC |
| 5930 | CAGUUCUUCCAUAGAAGGAAG | 11595 | CAGTTCTTCCATAGAAGGAAG |
| 5931 | CAGUUUCAACCAAUAUACAAA | 11596 | CAGTTTCAACCAATATACAAA |
| 5932 | CAGUUUCCAUCAGAGCACUGG | 11597 | CAGTTTCCATCAGAGCACTGG |
| 5933 | CAGUUUUCCUCGUCAGAUUUG | 11598 | CAGTTTTCCTCGTCAGATTTG |
| 5934 | CAUAAAGAAGAGGCAUUUCAG | 11599 | CATAAAGAAGAGGCATTTCAG |
| 5935 | CAUAAAGACGACAAUGGCAGU | 11600 | CATAAAGACGACAATGGCAGT |
| 5936 | CAUAACAAAAAAUCCCCACUA | 11601 | CATAACAAAAAATCCCCACTA |
| 5937 | CAUAAUCAUAGCUCACGACAG | 11602 | CATAATCATAGCTCACGACAG |
| 5938 | CAUACAACGGGGACAUCAUUC | 11603 | CATACAACGGGGACATCATTC |
| 5939 | CAUACAUACAACGGGGACAUC | 11604 | CATACATACAACGGGGACATC |
| 5940 | CAUACCCAUCAACGACAAGAU | 11605 | CATACCCATCAACGACAAGAT |
| 5941 | CAUAGAAGAGGUAAACACGUU | 11606 | CATAGAAGAGGTAAACACGTT |
| 5942 | CAUAGAAGGAAGACCCCCAGG | 11607 | CATAGAAGGAAGACCCCCAGG |
| 5943 | CAUAGAAGUGGAAGCACUAGG | 11608 | CATAGAAGTGGAAGCACTAGG |
| 5944 | CAUAGCUCACGACAGCCUCAA | 11609 | CATAGCTCACGACAGCCTCAA |
| 5945 | CAUAGGAAGAGACGCCGUGGG | 11610 | CATAGGAAGAGACGCCGTGGG |
| 5946 | CAUAUCCUCUGGGGACAGUAG | 11611 | CATATCCTCTGGGGACAGTAG |
| 5947 | CAUAUUCCCGGUCACACUGCC | 11612 | CATATTCCCGGTCACACTGCC |
| 5948 | CAUCAACGACAAGAUUGGGGA | 11613 | CATCAACGACAAGATTGGGGA |
| 5949 | CAUCACAGCGCCAGCUGGAGU | 11614 | CATCACAGCGCCAGCTGGAGT |
| 5950 | CAUCAGAGCACUGGAAUUCGU | 11615 | CATCAGAGCACTGGAATTCGT |
| 5951 | CAUCAGAGCCAUCCUGGCACU | 11616 | CATCAGAGCCATCCTGGCACT |
| 5952 | CAUCAGUGCAACAGUAACACG | 11617 | CATCAGTGCAACAGTAACACG |
| 5953 | CAUCAUUCUCCCAAAAAGCGU | 11618 | CATCATTCTCCCAAAAAGCGT |
| 5954 | CAUCAUUUGGUGAAUGAGUUU | 11619 | CATCATTTGGTGAATGAGTTT |
| 5955 | CAUCCCAACACACACGACAGA | 11620 | CATCCCAACACACACGACAGA |
| 5956 | CAUCCCCUCCCCUUUCAUCUU | 11621 | CATCCCCTCCCCTTTCATCTT |
| 5957 | CAUCCGAGCCAUCUUCGCAGU | 11622 | CATCCGAGCCATCTTCGCAGT |
| 5958 | CAUCCUCCAAGAUGGUCUUCC | 11623 | CATCCTCCAAGATGGTCTTCC |
| 5959 | CAUCCUCCAGACUGACCAUCG | 11624 | CATCCTCCAGACTGACCATCG |
| 5960 | CAUCCUCCAGACUGACCAUCU | 11625 | CATCCTCCAGACTGACCATCT |
| 5961 | CAUCCUCCCACCUCAGCCUCG | 11626 | CATCCTCCCACCTCAGCCTCG |
| 5962 | CAUCCUCUGUGGUCUUCUGAU | 11627 | CATCCTCTGTGGTCTTCTGAT |
| 5963 | CAUCCUGGCACUCAGCGCUGC | 11628 | CATCCTGGCACTCAGCGCTGC |
| 5964 | CAUCCUGGUCCCCCCGGGCCU | 11629 | CATCCTGGTCCCCCCGGGCCT |
| 5965 | CAUCGAGGGGUAGCUGUAGCC | 11630 | CATCGAGGGGTAGCTGTAGCC |
| 5966 | CAUCGAUAUCGCACUCUUUGA | 11631 | CATCGATATCGCACTCTTTGA |
| 5967 | CAUCGAUAUCUUCGCAUCUUC | 11632 | CATCGATATCTTCGCATCTTC |
| 5968 | CAUCGAUGCUUGAGAUGGAGU | 11633 | CATCGATGCTTGAGATGGAGT |
| 5969 | CAUCGCACCUCCAGAACUGAG | 11634 | CATCGCACCTCCAGAACTGAG |
| 5970 | CAUCGCAGACCCACUUGUAGG | 11635 | CATCGCAGACCCACTTGTAGG |
| 5971 | CAUCGCUCAUGUCCUUGCAGU | 11636 | CATCGCTCATGTCCTTGCAGT |
| 5972 | CAUCGGAACCUGUGAGGCGGU | 11637 | CATCGGAACCTGTGAGGCGGT |
| 5973 | CAUCGUGGCAGCGAAACUCGU | 11638 | CATCGTGGCAGCGAAACTCGT |
| 5974 | CAUCUCCCAGAAGCCACUCAU | 11639 | CATCTCCCAGAAGCCACTCAT |
| 5975 | CAUCUGACCAGUCCCGGCAGU | 11640 | CATCTGACCAGTCCCGGCAGT |
| 5976 | CAUCUGCUGUUGUGUGUUGAA | 11641 | CATCTGCTGTTGTGTGTTGAA |
| 5977 | CAUCUGUCGCCCACGUGGCUG | 11642 | CATCTGTCGCCCACGTGGCTG |
| 5978 | CAUCUGUCGCCCACUGCAGUC | 11643 | CATCTGTCGCCCACTGCAGTC |
| 5979 | CAUCUGUCUCGAGGGGUAGCU | 11644 | CATCTGTCTCGAGGGGTAGCT |
| 5980 | CAUCUGUUGCCCAGGCUGGAG | 11645 | CATCTGTTGCCCAGGCTGGAG |
| 5981 | CAUCUUCACGCGGGAGUCUGA | 11646 | CATCTTCACGCGGGAGTCTGA |
| 5982 | CAUCUUCCAUGUUUGAGGAUU | 11647 | CATCTTCCATGTTTGAGGATT |
| 5983 | CAUCUUCCUGACCUCGUGCCG | 11648 | CATCTTCCTGACCTCGTGCCG |
| 5984 | CAUCUUCGCAGUCGGGGUCGU | 11649 | CATCTTCGCAGTCGGGGTCGT |
| 5985 | CAUCUUCGCUGGGCCACCAGC | 11650 | CATCTTCGCTGGGCCACCAGC |
| 5986 | CAUGAACAGGAUCCACCACGA | 11651 | CATGAACAGGATCCACCACGA |
| 5987 | CAUGAAGCCAUGAACAGGAUC | 11652 | CATGAAGCCATGAACAGGATC |
| 5988 | CAUGAAUCCCUUUCCUGGCUU | 11653 | CATGAATCCCTTTCCTGGCTT |
| 5989 | CAUGAUCCACCCGCCUCGGCC | 11654 | CATGATCCACCCGCCTCGGCC |
| 5990 | CAUGAUUCGGGGGUGACAGGG | 11655 | CATGATTCGGGGGTGACAGGG |
| 5991 | CAUGCAUGGUUUUUUUUUGUU | 11656 | CATGCATGGTTTTTTTTTGTT |
| 5992 | CAUGCCGUCCGGGCAGGCGCA | 11657 | CATGCCGTCCGGGCAGGCGCA |
| 5993 | CAUGCCUGGCCAGAGGCCAUG | 11658 | CATGCCTGGCCAGAGGCCATG |
| 5994 | CAUGCUCGCAGCCUCUGCCAG | 11659 | CATGCTCGCAGCCTCTGCCAG |
| 5995 | CAUGGAUGCAGUUUCCAUCAG | 11660 | CATGGATGCAGTTTCCATCAG |
| 5996 | CAUGGCUCACUGCAACCUCCA | 11661 | CATGGCTCACTGCAACCTCCA |
| 5997 | CAUGGGCACUGUCCGAAGCCU | 11662 | CATGGGCACTGTCCGAAGCCT |
| 5998 | CAUGGGCUGCUGAUGCACCAU | 11663 | CATGGGCTGCTGATGCACCAT |
| 5999 | CAUGGUUUUUUUUUGUUUUUU | 11664 | CATGGTTTTTTTTTGTTTTTT |
| 6000 | CAUGUCCCUGGCCAGCAGCAU | 11665 | CATGTCCCTGGCCAGCAGCAT |
| 6001 | CAUGUCCUUGCAGUCAUAUUC | 11666 | CATGTCCTTGCAGTCATATTC |
| 6002 | CAUGUUGCAGACUUUGUCCAG | 11667 | CATGTTGCAGACTTTGTCCAG |
| 6003 | CAUGUUUGAGGAUUGUGUCAC | 11668 | CATGTTTGAGGATTGTGTCAC |
| 6004 | CAUGUUUUAAACACAUACCCA | 11669 | CATGTTTTAAACACATACCCA |
| 6005 | CAUUAACGCAGCCAACUUCAU | 11670 | CATTAACGCAGCCAACTTCAT |
| 6006 | CAUUCAGGCCCCCUUUCUUGA | 11671 | CATTCAGGCCCCCTTTCTTGA |
| 6007 | CAUUCAGGCUACAAAAACCUU | 11672 | CATTCAGGCTACAAAAACCTT |
| 6008 | CAUUCAUUGACACGGGCUUUC | 11673 | CATTCATTGACACGGGCTTTC |
| 6009 | CAUUCGCCGCUGUGACACUUG | 11674 | CATTCGCCGCTGTGACACTTG |
| 6010 | CAUUCGUUGGUCCCGCACUCU | 11675 | CATTCGTTGGTCCCGCACTCT |
| 6011 | CAUUCUCCCAAAAAGCGUUGC | 11676 | CATTCTCCCAAAAAGCGTTGC |
| 6012 | CAUUCUCCUGCCUCAGCCUCC | 11677 | CATTCTCCTGCCTCAGCCTCC |
| 6013 | CAUUCUCUGGGACAGGUCAGA | 11678 | CATTCTCTGGGACAGGTCAGA |
| 6014 | CAUUGACACGGGCUUUCCCUG | 11679 | CATTGACACGGGCTTTCCCTG |
| 6015 | CAUUGCAGACGUGGGAACAGC | 11680 | CATTGCAGACGTGGGAACAGC |
| 6016 | CAUUGCAUGGGCACUGUCCGA | 11681 | CATTGCATGGGCACTGTCCGA |
| 6017 | CAUUGCUCAGGGUGGUCCUCU | 11682 | CATTGCTCAGGGTGGTCCTCT |
| 6018 | CAUUGGGCCACUGAAUGUUUU | 11683 | CATTGGGCCACTGAATGTTTT |
| 6019 | CAUUGUCACUAUCUCCACCGU | 11684 | CATTGTCACTATCTCCACCGT |
| 6020 | CAUUUAAGAAAUUUAAUUGAU | 11685 | CATTTAAGAAATTTAATTGAT |
| 6021 | CAUUUACGUGCUCCGAAACCA | 11686 | CATTTACGTGCTCCGAAACCA |
| 6022 | CAUUUCAGAGGCAAUAACCCC | 11687 | CATTTCAGAGGCAATAACCCC |
| 6023 | CAUUUCCCGUCUUGGCACUGG | 11688 | CATTTCCCGTCTTGGCACTGG |
| 6024 | CAUUUCCGACGCCAUGAAUCC | 11689 | CATTTCCGACGCCATGAATCC |
| 6025 | CAUUUCCUCUGCCAGCAACGU | 11690 | CATTTCCTCTGCCAGCAACGT |
| 6026 | CAUUUGCAUAGAAGUGGAAGC | 11691 | CATTTGCATAGAAGTGGAAGC |
| 6027 | CAUUUGGUGAAUGAGUUUGGU | 11692 | CATTTGGTGAATGAGTTTGGT |
| 6028 | CAUUUUUUAAGAGAUGGGGUC | 11693 | CATTTTTTAAGAGATGGGGTC |
| 6029 | CCAAAAAGCGUUGCACACAGU | 11694 | CCAAAAAGCGTTGCACACAGT |
| 6030 | CCAAAAUACUUUGUCCUCAAA | 11695 | CCAAAATACTTTGTCCTCAAA |
| 6031 | CCAAAAUCCCAACCCAAGCCA | 11696 | CCAAAATCCCAACCCAAGCCA |
| 6032 | CCAAAGUGCUGGGAUUACAGG | 11697 | CCAAAGTGCTGGGATTACAGG |
| 6033 | CCAAAUGUACACAGUGUACAA | 11698 | CCAAATGTACACAGTGTACAA |
| 6034 | CCAAAUUCAUUUUUUAAGAGA | 11699 | CCAAATTCATTTTTTAAGAGA |
| 6035 | CCAACAAGUUGACAUCGGAAC | 11700 | CCAACAAGTTGACATCGGAAC |
| 6036 | CCAACACACACGACAGAAAAC | 11701 | CCAACACACACGACAGAAAAC |
| 6037 | CCAACCCAAGCCAUUGCAUGG | 11702 | CCAACCCAAGCCATTGCATGG |
| 6038 | CCAACUCCUGAACUCAAGUGA | 11703 | CCAACTCCTGAACTCAAGTGA |
| 6039 | CCAACUUCAUCGCUCAUGUCC | 11704 | CCAACTTCATCGCTCATGTCC |
| 6040 | CCAAGAUGGUCUUCCGGUUGC | 11705 | CCAAGATGGTCTTCCGGTTGC |
| 6041 | CCAAGCAGUCCCGGUCUGAGU | 11706 | CCAAGCAGTCCCGGTCTGAGT |
| 6042 | CCAAGCAUUCGUUGGUCCCGC | 11707 | CCAAGCATTCGTTGGTCCCGC |
| 6043 | CCAAGCCAUUGCAUGGGCACU | 11708 | CCAAGCCATTGCATGGGCACT |
| 6044 | CCAAGGAGAAGGGGUGGGCCA | 11709 | CCAAGGAGAAGGGGTGGGCCA |
| 6045 | CCAAGGAGGGGGGGGGAGAUU | 11710 | CCAAGGAGGGGGGGGGAGATT |
| 6046 | CCAAGGUAACCGGGUGUCUCA | 11711 | CCAAGGTAACCGGGTGTCTCA |
| 6047 | CCAAGUAGCUGGGAUCACAGG | 11712 | CCAAGTAGCTGGGATCACAGG |
| 6048 | CCAAUAUACAAAGUGCAAACA | 11713 | CCAATATACAAAGTGCAAACA |
| 6049 | CCAAUCCCUUGUGACAUCUUC | 11714 | CCAATCCCTTGTGACATCTTC |
| 6050 | CCACACCAUUCAGGCCCCCUU | 11715 | CCACACCATTCAGGCCCCCTT |
| 6051 | CCACACCUGGGUGCAGGCCAC | 11716 | CCACACCTGGGTGCAGGCCAC |
| 6052 | CCACAGAGCACAGCGGAAACC | 11717 | CCACAGAGCACAGCGGAAACC |
| 6053 | CCACAGCCAGCCCGUCGGGGG | 11718 | CCACAGCCAGCCCGTCGGGGG |
| 6054 | CCACAGCCUUGCAGGCCUUCG | 11719 | CCACAGCCTTGCAGGCCTTCG |
| 6055 | CCACAGCGCAGUUUUCCUCGU | 11720 | CCACAGCGCAGTTTTCCTCGT |
| 6056 | CCACAGCUGAAGUCCCCGGAU | 11721 | CCACAGCTGAAGTCCCCGGAT |
| 6057 | CCACAGCUGGGGGAUGCAGGU | 11722 | CCACAGCTGGGGGATGCAGGT |
| 6058 | CCACAGGACAGCCUUGCUCGU | 11723 | CCACAGGACAGCCTTGCTCGT |
| 6059 | CCACAGGUGAGCACCGGGCAG | 11724 | CCACAGGTGAGCACCGGGCAG |
| 6060 | CCACCACGAUGGCCCUUGGCU | 11725 | CCACCACGATGGCCCTTGGCT |
| 6061 | CCACCACGCCCGGCUAAUUUU | 11726 | CCACCACGCCCGGCTAATTTT |
| 6062 | CCACCACGGAUUCAGCCAGAU | 11727 | CCACCACGGATTCAGCCAGAT |
| 6063 | CCACCAGCUGGAAGCCGUCGG | 11728 | CCACCAGCTGGAAGCCGTCGG |
| 6064 | CCACCAUCACAGCGCCAGCUG | 11729 | CCACCATCACAGCGCCAGCTG |
| 6065 | CCACCCGCCUCGGCCUCCCAA | 11730 | CCACCCGCCTCGGCCTCCCAA |
| 6066 | CCACCCUCCAGGUUCACGCAG | 11731 | CCACCCTCCAGGTTCACGCAG |
| 6067 | CCACCGUACCCAGCUGAUUUU | 11732 | CCACCGTACCCAGCTGATTTT |
| 6068 | CCACCGUGGUGAGCCCAGGGG | 11733 | CCACCGTGGTGAGCCCAGGGG |
| 6069 | CCACCUCAGCCUCGCCAUGCC | 11734 | CCACCTCAGCCTCGCCATGCC |
| 6070 | CCACCUCCGUGUCCAGAGCGA | 11735 | CCACCTCCGTGTCCAGAGCGA |
| 6071 | CCACCUCCUGGGCUCAGGCGA | 11736 | CCACCTCCTGGGCTCAGGCGA |
| 6072 | CCACCUCGAAAGACACUGGGU | 11737 | CCACCTCGAAAGACACTGGGT |
| 6073 | CCACGAUGGCCCUUGGCUUGG | 11738 | CCACGATGGCCCTTGGCTTGG |
| 6074 | CCACGCCCGGCUAAUUUUUUG | 11739 | CCACGCCCGGCTAATTTTTTG |
| 6075 | CCACGGAUUCAGCCAGAUCAU | 11740 | CCACGGATTCAGCCAGATCAT |
| 6076 | CCACGGGAAUGGAAGUGGGUA | 11741 | CCACGGGAATGGAAGTGGGTA |
| 6077 | CCACGUCAUCCUCCAGACUGA | 11742 | CCACGTCATCCTCCAGACTGA |
| 6078 | CCACGUGGCUGAAGAUCUCGG | 11743 | CCACGTGGCTGAAGATCTCGG |
| 6079 | CCACGUGUCCUCACGGCCAAG | 11744 | CCACGTGTCCTCACGGCCAAG |
| 6080 | CCACGUUCCUCAGGUUGGGGA | 11745 | CCACGTTCCTCAGGTTGGGGA |
| 6081 | CCACUAUCUGUACAGGGACGC | 11746 | CCACTATCTGTACAGGGACGC |
| 6082 | CCACUCAUACAUACAACGGGG | 11747 | CCACTCATACATACAACGGGG |
| 6083 | CCACUCAUCCGAGCCAUCUUC | 11748 | CCACTCATCCGAGCCATCTTC |
| 6084 | CCACUGAAUGUUUUCAGUCAC | 11749 | CCACTGAATGTTTTCAGTCAC |
| 6085 | CCACUGAGGAGAUCUAGGGUG | 11750 | CCACTGAGGAGATCTAGGGTG |
| 6086 | CCACUGCAGCCUCAGCCUCUC | 11751 | CCACTGCAGCCTCAGCCTCTC |
| 6087 | CCACUGCAGCCUCAGCCUCUG | 11752 | CCACTGCAGCCTCAGCCTCTG |
| 6088 | CCACUGCAGUCCCCGCCGCGG | 11753 | CCACTGCAGTCCCCGCCGCGG |
| 6089 | CCACUGCGCCCGGCCUCACCG | 11754 | CCACTGCGCCCGGCCTCACCG |
| 6090 | CCACUUAGGCAGUGGAACUCG | 11755 | CCACTTAGGCAGTGGAACTCG |
| 6091 | CCACUUGCUUCUCUGGGCCUG | 11756 | CCACTTGCTTCTCTGGGCCTG |
| 6092 | CCACUUGGCCAUCGCACCUCC | 11757 | CCACTTGGCCATCGCACCTCC |
| 6093 | CCACUUGUAGGAGAUGCAUUU | 11758 | CCACTTGTAGGAGATGCATTT |
| 6094 | CCAGAAAGGCUUAUCACGGUU | 11759 | CCAGAAAGGCTTATCACGGTT |
| 6095 | CCAGAACUGAGGAAUGCAGCG | 11760 | CCAGAACTGAGGAATGCAGCG |
| 6096 | CCAGAAGCCACUCAUACAUAC | 11761 | CCAGAAGCCACTCATACATAC |
| 6097 | CCAGACUGACCAUCGAGGGGU | 11762 | CCAGACTGACCATCGAGGGGT |
| 6098 | CCAGACUGACCAUCUGUCUCG | 11763 | CCAGACTGACCATCTGTCTCG |
| 6099 | CCAGACUGCGGACUGCAGUGG | 11764 | CCAGACTGCGGACTGCAGTGG |
| 6100 | CCAGACUGGAGGAUUUAUUUA | 11765 | CCAGACTGGAGGATTTATTTA |
| 6101 | CCAGAGACCCAGCUCCGUGGC | 11766 | CCAGAGACCCAGCTCCGTGGC |
| 6102 | CCAGAGCGACCACGUUCCUCA | 11767 | CCAGAGCGACCACGTTCCTCA |
| 6103 | CCAGAGCUUGGUGAGACAUUG | 11768 | CCAGAGCTTGGTGAGACATTG |
| 6104 | CCAGAGGCCAUGGGCUGCUGA | 11769 | CCAGAGGCCATGGGCTGCTGA |
| 6105 | CCAGAUCAUUUCCGACGCCAU | 11770 | CCAGATCATTTCCGACGCCAT |
| 6106 | CCAGAUGAAUAAAUAUAUAAA | 11771 | CCAGATGAATAAATATATAAA |
| 6107 | CCAGCAACGUCGCCCAGAGCU | 11772 | CCAGCAACGTCGCCCAGAGCT |
| 6108 | CCAGCAGCAUGCCGUCCGGGC | 11773 | CCAGCAGCATGCCGTCCGGGC |
| 6109 | CCAGCCCCAGGGCCCCAUGCU | 11774 | CCAGCCCCAGGGCCCCATGCT |
| 6110 | CCAGCCCGUCGGGGGCCUGGA | 11775 | CCAGCCCGTCGGGGGCCTGGA |
| 6111 | CCAGCCUCUUUUCAUCCUCCA | 11776 | CCAGCCTCTTTTCATCCTCCA |
| 6112 | CCAGCGAGUAGAUGUCCACAC | 11777 | CCAGCGAGTAGATGTCCACAC |
| 6113 | CCAGCGCAAUUUCCAGCCCCA | 11778 | CCAGCGCAATTTCCAGCCCCA |
| 6114 | CCAGCGUCAUCUUCCUGACCU | 11779 | CCAGCGTCATCTTCCTGACCT |
| 6115 | CCAGCUCAGCCUCCCAAAGUG | 11780 | CCAGCTCAGCCTCCCAAAGTG |
| 6116 | CCAGCUCCGUGGCGAUGGGGA | 11781 | CCAGCTCCGTGGCGATGGGGA |
| 6117 | CCAGCUGAUUUUUAACUUUUU | 11782 | CCAGCTGATTTTTAACTTTTT |
| 6118 | CCAGCUGGAAGCCGUCGGGGC | 11783 | CCAGCTGGAAGCCGTCGGGGC |
| 6119 | CCAGCUGGAAGCCUUCCUCAC | 11784 | CCAGCTGGAAGCCTTCCTCAC |
| 6120 | CCAGCUGGAGUGGAUGCACUC | 11785 | CCAGCTGGAGTGGATGCACTC |
| 6121 | CCAGGACAAGGUGGGGACGGA | 11786 | CCAGGACAAGGTGGGGACGGA |
| 6122 | CCAGGACAGAGUCGGUCCAGU | 11787 | CCAGGACAGAGTCGGTCCAGT |
| 6123 | CCAGGAGGGCCCAGGAUAACG | 11788 | CCAGGAGGGCCCAGGATAACG |
| 6124 | CCAGGAUAACGGAGGGCGACU | 11789 | CCAGGATAACGGAGGGCGACT |
| 6125 | CCAGGAUGGUCUCGAUCUCCU | 11790 | CCAGGATGGTCTCGATCTCCT |
| 6126 | CCAGGCAAAGGAAGACGAGGA | 11791 | CCAGGCAAAGGAAGACGAGGA |
| 6127 | CCAGGCAGAUGUUCACGCCAC | 11792 | CCAGGCAGATGTTCACGCCAC |
| 6128 | CCAGGCAGCCGGGAGGUGUCG | 11793 | CCAGGCAGCCGGGAGGTGTCG |
| 6129 | CCAGGCAGUGUCCCGACCCGG | 11794 | CCAGGCAGTGTCCCGACCCGG |
| 6130 | CCAGGCCAGACUGCGGACUGC | 11795 | CCAGGCCAGACTGCGGACTGC |
| 6131 | CCAGGCUGGAAAGCAGUGGCA | 11796 | CCAGGCTGGAAAGCAGTGGCA |
| 6132 | CCAGGCUGGAGUGCAGUGGCU | 11797 | CCAGGCTGGAGTGCAGTGGCT |
| 6133 | CCAGGCUGGUGUCCUGACAGC | 11798 | CCAGGCTGGTGTCCTGACAGC |
| 6134 | CCAGGGCCCCAUGCUCGCAGC | 11799 | CCAGGGCCCCATGCTCGCAGC |
| 6135 | CCAGGGGGUCCCGUUGGUGCC | 11800 | CCAGGGGGTCCCGTTGGTGCC |
| 6136 | CCAGGGGUGGCCCCAGGCAGC | 11801 | CCAGGGGTGGCCCCAGGCAGC |
| 6137 | CCAGGGUGAUGCAUUCGCCGC | 11802 | CCAGGGTGATGCATTCGCCGC |
| 6138 | CCAGGGUUUGCAAAAAUAAAU | 11803 | CCAGGGTTTGCAAAAATAAAT |
| 6139 | CCAGGUUCACGCAGAGCUGGC | 11804 | CCAGGTTCACGCAGAGCTGGC |
| 6140 | CCAGUACAUGAAGCCAUGAAC | 11805 | CCAGTACATGAAGCCATGAAC |
| 6141 | CCAGUAGAGGCGGCCACUGAG | 11806 | CCAGTAGAGGCGGCCACTGAG |
| 6142 | CCAGUAGAUGUUGCUGUGGAU | 11807 | CCAGTAGATGTTGCTGTGGAT |
| 6143 | CCAGUAGAUUCUAUUGCUGGC | 11808 | CCAGTAGATTCTATTGCTGGC |
| 6144 | CCAGUCAGUCCAGUACAUGAA | 11809 | CCAGTCAGTCCAGTACATGAA |
| 6145 | CCAGUCCACAGCCAGCCCGUC | 11810 | CCAGTCCACAGCCAGCCCGTC |
| 6146 | CCAGUCCCGGCAGUCUCUAGC | 11811 | CCAGTCCCGGCAGTCTCTAGC |
| 6147 | CCAGUGACUCAGGCUCUGCCU | 11812 | CCAGTGACTCAGGCTCTGCCT |
| 6148 | CCAGUUCACUCCUCUUGGCUG | 11813 | CCAGTTCACTCCTCTTGGCTG |
| 6149 | CCAGUUCGUGCAAAUAAUCUU | 11814 | CCAGTTCGTGCAAATAATCTT |
| 6150 | CCAGUUCUUCCAUAGAAGGAA | 11815 | CCAGTTCTTCCATAGAAGGAA |
| 6151 | CCAUAGAAGGAAGACCCCCAG | 11816 | CCATAGAAGGAAGACCCCCAG |
| 6152 | CCAUAUCCUCUGGGGACAGUA | 11817 | CCATATCCTCTGGGGACAGTA |
| 6153 | CCAUCAACGACAAGAUUGGGG | 11818 | CCATCAACGACAAGATTGGGG |
| 6154 | CCAUCACAGCGCCAGCUGGAG | 11819 | CCATCACAGCGCCAGCTGGAG |
| 6155 | CCAUCAGAGCACUGGAAUUCG | 11820 | CCATCAGAGCACTGGAATTCG |
| 6156 | CCAUCCCAACACACACGACAG | 11821 | CCATCCCAACACACACGACAG |
| 6157 | CCAUCCUCCCACCUCAGCCUC | 11822 | CCATCCTCCCACCTCAGCCTC |
| 6158 | CCAUCCUGGCACUCAGCGCUG | 11823 | CCATCCTGGCACTCAGCGCTG |
| 6159 | CCAUCGAGGGGUAGCUGUAGC | 11824 | CCATCGAGGGGTAGCTGTAGC |
| 6160 | CCAUCGCACCUCCAGAACUGA | 11825 | CCATCGCACCTCCAGAACTGA |
| 6161 | CCAUCGCAGACCCACUUGUAG | 11826 | CCATCGCAGACCCACTTGTAG |
| 6162 | CCAUCGUGGCAGCGAAACUCG | 11827 | CCATCGTGGCAGCGAAACTCG |
| 6163 | CCAUCUCCCAGAAGCCACUCA | 11828 | CCATCTCCCAGAAGCCACTCA |
| 6164 | CCAUCUGCUGUUGUGUGUUGA | 11829 | CCATCTGCTGTTGTGTGTTGA |
| 6165 | CCAUCUGUCUCGAGGGGUAGC | 11830 | CCATCTGTCTCGAGGGGTAGC |
| 6166 | CCAUCUUCGCAGUCGGGGUCG | 11831 | CCATCTTCGCAGTCGGGGTCG |
| 6167 | CCAUGAACAGGAUCCACCACG | 11832 | CCATGAACAGGATCCACCACG |
| 6168 | CCAUGAAUCCCUUUCCUGGCU | 11833 | CCATGAATCCCTTTCCTGGCT |
| 6169 | CCAUGCAUGGUUUUUUUUUGU | 11834 | CCATGCATGGTTTTTTTTTGT |
| 6170 | CCAUGCCUGGCCAGAGGCCAU | 11835 | CCATGCCTGGCCAGAGGCCAT |
| 6171 | CCAUGCUCGCAGCCUCUGCCA | 11836 | CCATGCTCGCAGCCTCTGCCA |
| 6172 | CCAUGGAUGCAGUUUCCAUCA | 11837 | CCATGGATGCAGTTTCCATCA |
| 6173 | CCAUGGGCUGCUGAUGCACCA | 11838 | CCATGGGCTGCTGATGCACCA |
| 6174 | CCAUGUUGCAGACUUUGUCCA | 11839 | CCATGTTGCAGACTTTGTCCA |
| 6175 | CCAUGUUUGAGGAUUGUGUCA | 11840 | CCATGTTTGAGGATTGTGTCA |
| 6176 | CCAUUCAGGCCCCCUUUCUUG | 11841 | CCATTCAGGCCCCCTTTCTTG |
| 6177 | CCAUUCUCCUGCCUCAGCCUC | 11842 | CCATTCTCCTGCCTCAGCCTC |
| 6178 | CCAUUGCAUGGGCACUGUCCG | 11843 | CCATTGCATGGGCACTGTCCG |
| 6179 | CCAUUGCUCAGGGUGGUCCUC | 11844 | CCATTGCTCAGGGTGGTCCTC |
| 6180 | CCAUUGGGCCACUGAAUGUUU | 11845 | CCATTGGGCCACTGAATGTTT |
| 6181 | CCCAAAAAGCGUUGCACACAG | 11846 | CCCAAAAAGCGTTGCACACAG |
| 6182 | CCCAAAGUGCUGGGAUUACAG | 11847 | CCCAAAGTGCTGGGATTACAG |
| 6183 | CCCAACACACACGACAGAAAA | 11848 | CCCAACACACACGACAGAAAA |
| 6184 | CCCAACCCAAGCCAUUGCAUG | 11849 | CCCAACCCAAGCCATTGCATG |
| 6185 | CCCAAGCCAUUGCAUGGGCAC | 11850 | CCCAAGCCATTGCATGGGCAC |
| 6186 | CCCAAGUAGCUGGGAUCACAG | 11851 | CCCAAGTAGCTGGGATCACAG |
| 6187 | CCCACAGCCUUGCAGGCCUUC | 11852 | CCCACAGCCTTGCAGGCCTTC |
| 6188 | CCCACAGCUGAAGUCCCCGGA | 11853 | CCCACAGCTGAAGTCCCCGGA |
| 6189 | CCCACAGCUGGGGGAUGCAGG | 11854 | CCCACAGCTGGGGGATGCAGG |
| 6190 | CCCACCUCAGCCUCGCCAUGC | 11855 | CCCACCTCAGCCTCGCCATGC |
| 6191 | CCCACCUCGAAAGACACUGGG | 11856 | CCCACCTCGAAAGACACTGGG |
| 6192 | CCCACGUGGCUGAAGAUCUCG | 11857 | CCCACGTGGCTGAAGATCTCG |
| 6193 | CCCACUAUCUGUACAGGGACG | 11858 | CCCACTATCTGTACAGGGACG |
| 6194 | CCCACUGCAGUCCCCGCCGCG | 11859 | CCCACTGCAGTCCCCGCCGCG |
| 6195 | CCCACUUGUAGGAGAUGCAUU | 11860 | CCCACTTGTAGGAGATGCATT |
| 6196 | CCCAGAAGCCACUCAUACAUA | 11861 | CCCAGAAGCCACTCATACATA |
| 6197 | CCCAGAGCUUGGUGAGACAUU | 11862 | CCCAGAGCTTGGTGAGACATT |
| 6198 | CCCAGAUGAAUAAAUAUAUAA | 11863 | CCCAGATGAATAAATATATAA |
| 6199 | CCCAGCUCCGUGGCGAUGGGG | 11864 | CCCAGCTCCGTGGCGATGGGG |
| 6200 | CCCAGCUGAUUUUUAACUUUU | 11865 | CCCAGCTGATTTTTAACTTTT |
| 6201 | CCCAGGACAAGGUGGGGACGG | 11866 | CCCAGGACAAGGTGGGGACGG |
| 6202 | CCCAGGACAGAGUCGGUCCAG | 11867 | CCCAGGACAGAGTCGGTCCAG |
| 6203 | CCCAGGAUAACGGAGGGCGAC | 11868 | CCCAGGATAACGGAGGGCGAC |
| 6204 | CCCAGGCAAAGGAAGACGAGG | 11869 | CCCAGGCAAAGGAAGACGAGG |
| 6205 | CCCAGGCAGCCGGGAGGUGUC | 11870 | CCCAGGCAGCCGGGAGGTGTC |
| 6206 | CCCAGGCCAGACUGCGGACUG | 11871 | CCCAGGCCAGACTGCGGACTG |
| 6207 | CCCAGGCUGGAAAGCAGUGGC | 11872 | CCCAGGCTGGAAAGCAGTGGC |
| 6208 | CCCAGGCUGGAGUGCAGUGGC | 11873 | CCCAGGCTGGAGTGCAGTGGC |
| 6209 | CCCAGGGCCCCAUGCUCGCAG | 11874 | CCCAGGGCCCCATGCTCGCAG |
| 6210 | CCCAGGGGUGGCCCCAGGCAG | 11875 | CCCAGGGGTGGCCCCAGGCAG |
| 6211 | CCCAGUAGAGGCGGCCACUGA | 11876 | CCCAGTAGAGGCGGCCACTGA |
| 6212 | CCCAGUCAGUCCAGUACAUGA | 11877 | CCCAGTCAGTCCAGTACATGA |
| 6213 | CCCAUCAACGACAAGAUUGGG | 11878 | CCCATCAACGACAAGATTGGG |
| 6214 | CCCAUCCCAACACACACGACA | 11879 | CCCATCCCAACACACACGACA |
| 6215 | CCCAUCGUGGCAGCGAAACUC | 11880 | CCCATCGTGGCAGCGAAACTC |
| 6216 | CCCAUCUCCCAGAAGCCACUC | 11881 | CCCATCTCCCAGAAGCCACTC |
| 6217 | CCCAUGCUCGCAGCCUCUGCC | 11882 | CCCATGCTCGCAGCCTCTGCC |
| 6218 | CCCCACAGCUGAAGUCCCCGG | 11883 | CCCCACAGCTGAAGTCCCCGG |
| 6219 | CCCCACUAUCUGUACAGGGAC | 11884 | CCCCACTATCTGTACAGGGAC |
| 6220 | CCCCAGGACAAGGUGGGGACG | 11885 | CCCCAGGACAAGGTGGGGACG |
| 6221 | CCCCAGGCAAAGGAAGACGAG | 11886 | CCCCAGGCAAAGGAAGACGAG |
| 6222 | CCCCAGGCAGCCGGGAGGUGU | 11887 | CCCCAGGCAGCCGGGAGGTGT |
| 6223 | CCCCAGGGCCCCAUGCUCGCA | 11888 | CCCCAGGGCCCCATGCTCGCA |
| 6224 | CCCCAGUCAGUCCAGUACAUG | 11889 | CCCCAGTCAGTCCAGTACATG |
| 6225 | CCCCAUGCUCGCAGCCUCUGC | 11890 | CCCCATGCTCGCAGCCTCTGC |
| 6226 | CCCCCACAGCUGAAGUCCCCG | 11891 | CCCCCACAGCTGAAGTCCCCG |
| 6227 | CCCCCAGGACAAGGUGGGGAC | 11892 | CCCCCAGGACAAGGTGGGGAC |
| 6228 | CCCCCAGGCAAAGGAAGACGA | 11893 | CCCCCAGGCAAAGGAAGACGA |
| 6229 | CCCCCCAGGACAAGGUGGGGA | 11894 | CCCCCCAGGACAAGGTGGGGA |
| 6230 | CCCCCCGGGCCUGUUUCUCUU | 11895 | CCCCCCGGGCCTGTTTCTCTT |
| 6231 | CCCCCGAGACCAGAGACCCAG | 11896 | CCCCCGAGACCAGAGACCCAG |
| 6232 | CCCCCGGGCCUGUUUCUCUUA | 11897 | CCCCCGGGCCTGTTTCTCTTA |
| 6233 | CCCCCGUUGACAUCGAUGCUU | 11898 | CCCCCGTTGACATCGATGCTT |
| 6234 | CCCCCUACACAGGGCCCCGAG | 11899 | CCCCCTACACAGGGCCCCGAG |
| 6235 | CCCCCUUUCUUGAUCUUGGCG | 11900 | CCCCCTTTCTTGATCTTGGCG |
| 6236 | CCCCGAGAAUAGUCACUGAAC | 11901 | CCCCGAGAATAGTCACTGAAC |
| 6237 | CCCCGAGACCAGAGACCCAGC | 11902 | CCCCGAGACCAGAGACCCAGC |
| 6238 | CCCCGCCGCGGCGAGGAGCAA | 11903 | CCCCGCCGCGGCGAGGAGCAA |
| 6239 | CCCCGGAUUUGCAGGUGACAG | 11904 | CCCCGGATTTGCAGGTGACAG |
| 6240 | CCCCGGCAUUCAUUGACACGG | 11905 | CCCCGGCATTCATTGACACGG |
| 6241 | CCCCGGGCCUGUUUCUCUUAU | 11906 | CCCCGGGCCTGTTTCTCTTAT |
| 6242 | CCCCGUUGACAUCGAUGCUUG | 11907 | CCCCGTTGACATCGATGCTTG |
| 6243 | CCCCUACACAGGGCCCCGAGA | 11908 | CCCCTACACAGGGCCCCGAGA |
| 6244 | CCCCUACAGCGCUGCGGCCAC | 11909 | CCCCTACAGCGCTGCGGCCAC |
| 6245 | CCCCUCCCCUUUCAUCUUCCA | 11910 | CCCCTCCCCTTTCATCTTCCA |
| 6246 | CCCCUCUCUGUCCCCGGCAUU | 11911 | CCCCTCTCTGTCCCCGGCATT |
| 6247 | CCCCUCUGUGAACUUGAUCGG | 11912 | CCCCTCTGTGAACTTGATCGG |
| 6248 | CCCCUUGGAACACGUAAAGAC | 11913 | CCCCTTGGAACACGTAAAGAC |
| 6249 | CCCCUUUCAUCUUCCAUGUUU | 11914 | CCCCTTTCATCTTCCATGTTT |
| 6250 | CCCCUUUCUUGAUCUUGGCGG | 11915 | CCCCTTTCTTGATCTTGGCGG |
| 6251 | CCCGACCCGGAUCACGACCUG | 11916 | CCCGACCCGGATCACGACCTG |
| 6252 | CCCGAGAAUAGUCACUGAACA | 11917 | CCCGAGAATAGTCACTGAACA |
| 6253 | CCCGAGACCAGAGACCCAGCU | 11918 | CCCGAGACCAGAGACCCAGCT |
| 6254 | CCCGAGUAGCUGGGACUACAG | 11919 | CCCGAGTAGCTGGGACTACAG |
| 6255 | CCCGCACUCUUUGAUGGGUUC | 11920 | CCCGCACTCTTTGATGGGTTC |
| 6256 | CCCGCCACCACGCCCGGCUAA | 11921 | CCCGCCACCACGCCCGGCTAA |
| 6257 | CCCGCCGCGGCGAGGAGCAAG | 11922 | CCCGCCGCGGCGAGGAGCAAG |
| 6258 | CCCGCCUCGGCCUCCCAAAGU | 11923 | CCCGCCTCGGCCTCCCAAAGT |
| 6259 | CCCGGAUCACGACCUGCUGUG | 11924 | CCCGGATCACGACCTGCTGTG |
| 6260 | CCCGGAUUUGCAGGUGACAGA | 11925 | CCCGGATTTGCAGGTGACAGA |
| 6261 | CCCGGCAGUCUCUAGCCAUGU | 11926 | CCCGGCAGTCTCTAGCCATGT |
| 6262 | CCCGGCAUUCAUUGACACGGG | 11927 | CCCGGCATTCATTGACACGGG |
| 6263 | CCCGGCCUCACCGUGCAUGUU | 11928 | CCCGGCCTCACCGTGCATGTT |
| 6264 | CCCGGCUAAUUUUUUGUAUUU | 11929 | CCCGGCTAATTTTTTGTATTT |
| 6265 | CCCGGGACUUGUCGUCGCCUU | 11930 | CCCGGGACTTGTCGTCGCCTT |
| 6266 | CCCGGGCCUGUUUCUCUUAUC | 11931 | CCCGGGCCTGTTTCTCTTATC |
| 6267 | CCCGGGUUCACACCAUUCUCC | 11932 | CCCGGGTTCACACCATTCTCC |
| 6268 | CCCGGUCAACCUGCCCCUCUC | 11933 | CCCGGTCAACCTGCCCCTCTC |
| 6269 | CCCGGUCACACUGCCGGCUGC | 11934 | CCCGGTCACACTGCCGGCTGC |
| 6270 | CCCGGUCUGAGUCACAGACGA | 11935 | CCCGGTCTGAGTCACAGACGA |
| 6271 | CCCGUCAAACGAUCCAGACUG | 11936 | CCCGTCAAACGATCCAGACTG |
| 6272 | CCCGUCGGGGGCCUGGAUGUC | 11937 | CCCGTCGGGGGCCTGGATGTC |
| 6273 | CCCGUCUUGGCACUGGAACUC | 11938 | CCCGTCTTGGCACTGGAACTC |
| 6274 | CCCGUUGACAUCGAUGCUUGA | 11939 | CCCGTTGACATCGATGCTTGA |
| 6275 | CCCGUUGGUGCCAUCUGCUGU | 11940 | CCCGTTGGTGCCATCTGCTGT |
| 6276 | CCCUACACAGGGCCCCGAGAA | 11941 | CCCTACACAGGGCCCCGAGAA |
| 6277 | CCCUACAGCGCUGCGGCCACU | 11942 | CCCTACAGCGCTGCGGCCACT |
| 6278 | CCCUCACGCUACUGGGCUUCU | 11943 | CCCTCACGCTACTGGGCTTCT |
| 6279 | CCCUCCAGGUUCACGCAGAGC | 11944 | CCCTCCAGGTTCACGCAGAGC |
| 6280 | CCCUCCCCUUUCAUCUUCCAU | 11945 | CCCTCCCCTTTCATCTTCCAT |
| 6281 | CCCUCGCAGAGUGUCACAUUA | 11946 | CCCTCGCAGAGTGTCACATTA |
| 6282 | CCCUCGCAGAGUGUCAGACAG | 11947 | CCCTCGCAGAGTGTCAGACAG |
| 6283 | CCCUCUCUGUCCCCGGCAUUC | 11948 | CCCTCTCTGTCCCCGGCATTC |
| 6284 | CCCUCUGUGAACUUGAUCGGG | 11949 | CCCTCTGTGAACTTGATCGGG |
| 6285 | CCCUGAAUAACGUUUUCCUCU | 11950 | CCCTGAATAACGTTTTCCTCT |
| 6286 | CCCUGCACUGGGGGGGACACA | 11951 | CCCTGCACTGGGGGGGACACA |
| 6287 | CCCUGGCCAGCAGCAUGCCGU | 11952 | CCCTGGCCAGCAGCATGCCGT |
| 6288 | CCCUGGCCUGGGAUCCCAUCC | 11953 | CCCTGGCCTGGGATCCCATCC |
| 6289 | CCCUGGCUUCCCGCGAUUGCA | 11954 | CCCTGGCTTCCCGCGATTGCA |
| 6290 | CCCUGGGGACAAUUGCCAAUC | 11955 | CCCTGGGGACAATTGCCAATC |
| 6291 | CCCUUGGAACACGUAAAGACC | 11956 | CCCTTGGAACACGTAAAGACC |
| 6292 | CCCUUGGCUUGGAGCCGUUCU | 11957 | CCCTTGGCTTGGAGCCGTTCT |
| 6293 | CCCUUGGUAUCCGCAACAGAG | 11958 | CCCTTGGTATCCGCAACAGAG |
| 6294 | CCCUUGUGACAUCUUCACGCG | 11959 | CCCTTGTGACATCTTCACGCG |
| 6295 | CCCUUUCAUCUUCCAUGUUUG | 11960 | CCCTTTCATCTTCCATGTTTG |
| 6296 | CCCUUUCCUGGCUUAAGGAAG | 11961 | CCCTTTCCTGGCTTAAGGAAG |
| 6297 | CCCUUUCUUGAUCUUGGCGGG | 11962 | CCCTTTCTTGATCTTGGCGGG |
| 6298 | CCGAAACCAGAAAGGCUUAUC | 11963 | CCGAAACCAGAAAGGCTTATC |
| 6299 | CCGAAGCCUGUUCUGCCUCCC | 11964 | CCGAAGCCTGTTCTGCCTCCC |
| 6300 | CCGACCCGGAUCACGACCUGC | 11965 | CCGACCCGGATCACGACCTGC |
| 6301 | CCGACGCCAUGAAUCCCUUUC | 11966 | CCGACGCCATGAATCCCTTTC |
| 6302 | CCGAGAAUAGUCACUGAACAA | 11967 | CCGAGAATAGTCACTGAACAA |
| 6303 | CCGAGACCAGAGACCCAGCUC | 11968 | CCGAGACCAGAGACCCAGCTC |
| 6304 | CCGAGAGAUGCACUUCCCAUC | 11969 | CCGAGAGATGCACTTCCCATC |
| 6305 | CCGAGCAGGGGCUACUGUCCC | 11970 | CCGAGCAGGGGCTACTGTCCC |
| 6306 | CCGAGCCAUCUUCGCAGUCGG | 11971 | CCGAGCCATCTTCGCAGTCGG |
| 6307 | CCGAGGCCUUGAGGGUGGCCG | 11972 | CCGAGGCCTTGAGGGTGGCCG |
| 6308 | CCGAGUAGCUGGGACUACAGG | 11973 | CCGAGTAGCTGGGACTACAGG |
| 6309 | CCGAUCUUAAGGUCAUUGCAG | 11974 | CCGATCTTAAGGTCATTGCAG |
| 6310 | CCGCAACAGAGACAGUGCCCA | 11975 | CCGCAACAGAGACAGTGCCCA |
| 6311 | CCGCACUCUUUGAUGGGUUCA | 11976 | CCGCACTCTTTGATGGGTTCA |
| 6312 | CCGCCACCACGCCCGGCUAAU | 11977 | CCGCCACCACGCCCGGCTAAT |
| 6313 | CCGCCAGUUCUUCCAUAGAAG | 11978 | CCGCCAGTTCTTCCATAGAAG |
| 6314 | CCGCCAUUGCUCAGGGUGGUC | 11979 | CCGCCATTGCTCAGGGTGGTC |
| 6315 | CCGCCGCGGCGAGGAGCAAGG | 11980 | CCGCCGCGGCGAGGAGCAAGG |
| 6316 | CCGCCGUUGUUGUCCAAGCAU | 11981 | CCGCCGTTGTTGTCCAAGCAT |
| 6317 | CCGCCUCGGCCUCCCAAAGUG | 11982 | CCGCCTCGGCCTCCCAAAGTG |
| 6318 | CCGCGGAGGAACGCCAGGCCU | 11983 | CCGCGGAGGAACGCCAGGCCT |
| 6319 | CCGCGGCGAGGAGCAAGGCGA | 11984 | CCGCGGCGAGGAGCAAGGCGA |
| 6320 | CCGCUGUGACACUUGAACUUG | 11985 | CCGCTGTGACACTTGAACTTG |
| 6321 | CCGCUUCCCGGGUUCACACCA | 11986 | CCGCTTCCCGGGTTCACACCA |
| 6322 | CCGGACAUCAGUGCAACAGUA | 11987 | CCGGACATCAGTGCAACAGTA |
| 6323 | CCGGAUCACGACCUGCUGUGU | 11988 | CCGGATCACGACCTGCTGTGT |
| 6324 | CCGGAUUUGCAGGUGACAGAC | 11989 | CCGGATTTGCAGGTGACAGAC |
| 6325 | CCGGCAGUCUCUAGCCAUGUU | 11990 | CCGGCAGTCTCTAGCCATGTT |
| 6326 | CCGGCAUUCAUUGACACGGGC | 11991 | CCGGCATTCATTGACACGGGC |
| 6327 | CCGGCCUCACCGUGCAUGUUU | 11992 | CCGGCCTCACCGTGCATGTTT |
| 6328 | CCGGCGAGGUCUCAGGAAGGG | 11993 | CCGGCGAGGTCTCAGGAAGGG |
| 6329 | CCGGCUAAUUUUUUGUAUUUU | 11994 | CCGGCTAATTTTTTGTATTTT |
| 6330 | CCGGCUGCCAUGGAUGCAGUU | 11995 | CCGGCTGCCATGGATGCAGTT |
| 6331 | CCGGGACUUGUCGUCGCCUUU | 11996 | CCGGGACTTGTCGTCGCCTTT |
| 6332 | CCGGGAGGUGUCGGGAACAGG | 11997 | CCGGGAGGTGTCGGGAACAGG |
| 6333 | CCGGGCAGGAGGCCUCGUCUG | 11998 | CCGGGCAGGAGGCCTCGTCTG |
| 6334 | CCGGGCAGGCGCAGGUAAACU | 11999 | CCGGGCAGGCGCAGGTAAACT |
| 6335 | CCGGGCCUGUUUCUCUUAUCC | 12000 | CCGGGCCTGTTTCTCTTATCC |
| 6336 | CCGGGGACCCGGGACUUGUCG | 12001 | CCGGGGACCCGGGACTTGTCG |
| 6337 | CCGGGGACUUCGCCAGGAGGG | 12002 | CCGGGGACTTCGCCAGGAGGG |
| 6338 | CCGGGGGCCCCUCUGUGAACU | 12003 | CCGGGGGCCCCTCTGTGAACT |
| 6339 | CCGGGUGUCUCAGGCACUUAA | 12004 | CCGGGTGTCTCAGGCACTTAA |
| 6340 | CCGGGUUCACACCAUUCUCCU | 12005 | CCGGGTTCACACCATTCTCCT |
| 6341 | CCGGUCAACCUGCCCCUCUCU | 12006 | CCGGTCAACCTGCCCCTCTCT |
| 6342 | CCGGUCACACUGCCGGCUGCC | 12007 | CCGGTCACACTGCCGGCTGCC |
| 6343 | CCGGUCCAGCGUCAUCUUCCU | 12008 | CCGGTCCAGCGTCATCTTCCT |
| 6344 | CCGGUCUGAGUCACAGACGAA | 12009 | CCGGTCTGAGTCACAGACGAA |
| 6345 | CCGGUUGCCCCCGUUGACAUC | 12010 | CCGGTTGCCCCCGTTGACATC |
| 6346 | CCGGUUGGUGAAGAAGAGGUA | 12011 | CCGGTTGGTGAAGAAGAGGTA |
| 6347 | CCGUACCCAGCUGAUUUUUAA | 12012 | CCGTACCCAGCTGATTTTTAA |
| 6348 | CCGUCAAACGAUCCAGACUGG | 12013 | CCGTCAAACGATCCAGACTGG |
| 6349 | CCGUCCAAGCAGUCCCGGUCU | 12014 | CCGTCCAAGCAGTCCCGGTCT |
| 6350 | CCGUCCGGGCAGGCGCAGGUA | 12015 | CCGTCCGGGCAGGCGCAGGTA |
| 6351 | CCGUCCUGGUUGUGGCAAAUG | 12016 | CCGTCCTGGTTGTGGCAAATG |
| 6352 | CCGUCGGGGCACAGGCACUCG | 12017 | CCGTCGGGGCACAGGCACTCG |
| 6353 | CCGUCGGGGGCCUGGAUGUCU | 12018 | CCGTCGGGGGCCTGGATGTCT |
| 6354 | CCGUCUUGGCACUGGAACUCG | 12019 | CCGTCTTGGCACTGGAACTCG |
| 6355 | CCGUGCAUGUUUUAAACACAU | 12020 | CCGTGCATGTTTTAAACACAT |
| 6356 | CCGUGGCGAUGGGGAGGACAA | 12021 | CCGTGGCGATGGGGAGGACAA |
| 6357 | CCGUGGGCUCUGUCAAGCUGG | 12022 | CCGTGGGCTCTGTCAAGCTGG |
| 6358 | CCGUGGUGAGCCCAGGGGUGG | 12023 | CCGTGGTGAGCCCAGGGGTGG |
| 6359 | CCGUGUCCAGAGCGACCACGU | 12024 | CCGTGTCCAGAGCGACCACGT |
| 6360 | CCGUUCUCCCUGAAUAACGUU | 12025 | CCGTTCTCCCTGAATAACGTT |
| 6361 | CCGUUGACAUCGAUGCUUGAG | 12026 | CCGTTGACATCGATGCTTGAG |
| 6362 | CCGUUGGUGCCAUCUGCUGUU | 12027 | CCGTTGGTGCCATCTGCTGTT |
| 6363 | CCGUUGUCGCAGUCCACUUGG | 12028 | CCGTTGTCGCAGTCCACTTGG |
| 6364 | CCGUUGUUGUCCAAGCAUUCG | 12029 | CCGTTGTTGTCCAAGCATTCG |
| 6365 | CCUACACAGGGCCCCGAGAAU | 12030 | CCTACACAGGGCCCCGAGAAT |
| 6366 | CCUACAGCGCUGCGGCCACUC | 12031 | CCTACAGCGCTGCGGCCACTC |
| 6367 | CCUAGCUGGAAACCCUGGCUU | 12032 | CCTAGCTGGAAACCCTGGCTT |
| 6368 | CCUCAAAGACGGCCAAGGAGA | 12033 | CCTCAAAGACGGCCAAGGAGA |
| 6369 | CCUCAAAUGCCUGAGCUCAAA | 12034 | CCTCAAATGCCTGAGCTCAAA |
| 6370 | CCUCACACUGGCACUUGUAGC | 12035 | CCTCACACTGGCACTTGTAGC |
| 6371 | CCUCACCGUGCAUGUUUUAAA | 12036 | CCTCACCGTGCATGTTTTAAA |
| 6372 | CCUCACGCUACUGGGCUUCUU | 12037 | CCTCACGCTACTGGGCTTCTT |
| 6373 | CCUCACGGCCAAGGUAACCGG | 12038 | CCTCACGGCCAAGGTAACCGG |
| 6374 | CCUCAGCCUCCCAAGUAGCUG | 12039 | CCTCAGCCTCCCAAGTAGCTG |
| 6375 | CCUCAGCCUCCCGAGUAGCUG | 12040 | CCTCAGCCTCCCGAGTAGCTG |
| 6376 | CCUCAGCCUCGCCAUGCCUGG | 12041 | CCTCAGCCTCGCCATGCCTGG |
| 6377 | CCUCAGCCUCUCUUGGCUGGG | 12042 | CCTCAGCCTCTCTTGGCTGGG |
| 6378 | CCUCAGCCUCUGUGAGGCAGC | 12043 | CCTCAGCCTCTGTGAGGCAGC |
| 6379 | CCUCAGGUUGGGGAUGAGGCU | 12044 | CCTCAGGTTGGGGATGAGGCT |
| 6380 | CCUCAUCCUCUGUGGUCUUCU | 12045 | CCTCATCCTCTGTGGTCTTCT |
| 6381 | CCUCAUGAUCCACCCGCCUCG | 12046 | CCTCATGATCCACCCGCCTCG |
| 6382 | CCUCAUGUCCCUGGCCAGCAG | 12047 | CCTCATGTCCCTGGCCAGCAG |
| 6383 | CCUCCAAGAUGGUCUUCCGGU | 12048 | CCTCCAAGATGGTCTTCCGGT |
| 6384 | CCUCCACCUCCUGGGCUCAGG | 12049 | CCTCCACCTCCTGGGCTCAGG |
| 6385 | CCUCCAGAACUGAGGAAUGCA | 12050 | CCTCCAGAACTGAGGAATGCA |
| 6386 | CCUCCAGACUGACCAUCGAGG | 12051 | CCTCCAGACTGACCATCGAGG |
| 6387 | CCUCCAGACUGACCAUCUGUC | 12052 | CCTCCAGACTGACCATCTGTC |
| 6388 | CCUCCAGGUUCACGCAGAGCU | 12053 | CCTCCAGGTTCACGCAGAGCT |
| 6389 | CCUCCCAAAGUGCUGGGAUUA | 12054 | CCTCCCAAAGTGCTGGGATTA |
| 6390 | CCUCCCAAGUAGCUGGGAUCA | 12055 | CCTCCCAAGTAGCTGGGATCA |
| 6391 | CCUCCCACCUCAGCCUCGCCA | 12056 | CCTCCCACCTCAGCCTCGCCA |
| 6392 | CCUCCCAGAUGAAUAAAUAUA | 12057 | CCTCCCAGATGAATAAATATA |
| 6393 | CCUCCCCUUUCAUCUUCCAUG | 12058 | CCTCCCCTTTCATCTTCCATG |
| 6394 | CCUCCCGAGUAGCUGGGACUA | 12059 | CCTCCCGAGTAGCTGGGACTA |
| 6395 | CCUCCGUGUCCAGAGCGACCA | 12060 | CCTCCGTGTCCAGAGCGACCA |
| 6396 | CCUCCUGCCUCAGCCUCCCAA | 12061 | CCTCCTGCCTCAGCCTCCCAA |
| 6397 | CCUCCUGGGCUCAGGCGAUCC | 12062 | CCTCCTGGGCTCAGGCGATCC |
| 6398 | CCUCGAAAGACACUGGGUGGG | 12063 | CCTCGAAAGACACTGGGTGGG |
| 6399 | CCUCGCAGAGUGUCACAUUAA | 12064 | CCTCGCAGAGTGTCACATTAA |
| 6400 | CCUCGCAGAGUGUCAGACAGC | 12065 | CCTCGCAGAGTGTCAGACAGC |
| 6401 | CCUCGCCAUGCCUGGCCAGAG | 12066 | CCTCGCCATGCCTGGCCAGAG |
| 6402 | CCUCGGCCUCCCAAAGUGCUG | 12067 | CCTCGGCCTCCCAAAGTGCTG |
| 6403 | CCUCGUAAGGAACCGAGGCCU | 12068 | CCTCGTAAGGAACCGAGGCCT |
| 6404 | CCUCGUCAGAUUUGUCCUUGC | 12069 | CCTCGTCAGATTTGTCCTTGC |
| 6405 | CCUCGUCUGAGCCGUCCAAGC | 12070 | CCTCGTCTGAGCCGTCCAAGC |
| 6406 | CCUCGUGCCGGUUGGUGAAGA | 12071 | CCTCGTGCCGGTTGGTGAAGA |
| 6407 | CCUCUCACACCAGUUCACUCC | 12072 | CCTCTCACACCAGTTCACTCC |
| 6408 | CCUCUCUGUCCCCGGCAUUCA | 12073 | CCTCTCTGTCCCCGGCATTCA |
| 6409 | CCUCUCUUGGCUGGGUGAGGU | 12074 | CCTCTCTTGGCTGGGTGAGGT |
| 6410 | CCUCUGCCAGCAACGUCGCCC | 12075 | CCTCTGCCAGCAACGTCGCCC |
| 6411 | CCUCUGCCAGGCAGUGUCCCG | 12076 | CCTCTGCCAGGCAGTGTCCCG |
| 6412 | CCUCUGGGAAGUGGCAUCAUU | 12077 | CCTCTGGGAAGTGGCATCATT |
| 6413 | CCUCUGGGGACAGUAGGUUUU | 12078 | CCTCTGGGGACAGTAGGTTTT |
| 6414 | CCUCUGUGAACUUGAUCGGGG | 12079 | CCTCTGTGAACTTGATCGGGG |
| 6415 | CCUCUGUGAGGCAGCUCCUCA | 12080 | CCTCTGTGAGGCAGCTCCTCA |
| 6416 | CCUCUGUGGUCUUCUGAUAGA | 12081 | CCTCTGTGGTCTTCTGATAGA |
| 6417 | CCUCUUCACGCCCUUGGUAUC | 12082 | CCTCTTCACGCCCTTGGTATC |
| 6418 | CCUCUUGGCUGGGUGAGGUUG | 12083 | CCTCTTGGCTGGGTGAGGTTG |
| 6419 | CCUCUUUUCAUCCUCCAAGAU | 12084 | CCTCTTTTCATCCTCCAAGAT |
| 6420 | CCUGAACUCAAGUGAUCCAGC | 12085 | CCTGAACTCAAGTGATCCAGC |
| 6421 | CCUGAAGUCCCGUCAAACGAU | 12086 | CCTGAAGTCCCGTCAAACGAT |
| 6422 | CCUGAAUAACGUUUUCCUCUU | 12087 | CCTGAATAACGTTTTCCTCTT |
| 6423 | CCUGACACUCAUCGAUAUCGC | 12088 | CCTGACACTCATCGATATCGC |
| 6424 | CCUGACACUCAUCGAUAUCUU | 12089 | CCTGACACTCATCGATATCTT |
| 6425 | CCUGACAGCCACACCUGGGUG | 12090 | CCTGACAGCCACACCTGGGTG |
| 6426 | CCUGACAUCCCCUCCCCUUUC | 12091 | CCTGACATCCCCTCCCCTTTC |
| 6427 | CCUGACCUCAUGAUCCACCCG | 12092 | CCTGACCTCATGATCCACCCG |
| 6428 | CCUGACCUCGUGCCGGUUGGU | 12093 | CCTGACCTCGTGCCGGTTGGT |
| 6429 | CCUGACGGUGGAUGUCUCCUG | 12094 | CCTGACGGTGGATGTCTCCTG |
| 6430 | CCUGAGCUCAAACCAUCCUCC | 12095 | CCTGAGCTCAAACCATCCTCC |
| 6431 | CCUGAGUUUAGGGGUUUGGCU | 12096 | CCTGAGTTTAGGGGTTTGGCT |
| 6432 | CCUGCACUGGGGGGGACACAG | 12097 | CCTGCACTGGGGGGGACACAG |
| 6433 | CCUGCCCCUCUCUGUCCCCGG | 12098 | CCTGCCCCTCTCTGTCCCCGG |
| 6434 | CCUGCCUCAGCCUCCCAAGUA | 12099 | CCTGCCTCAGCCTCCCAAGTA |
| 6435 | CCUGCCUCAGCCUCCCGAGUA | 12100 | CCTGCCTCAGCCTCCCGAGTA |
| 6436 | CCUGCUGUGUCCUAGCUGGAA | 12101 | CCTGCTGTGTCCTAGCTGGAA |
| 6437 | CCUGGAGAGAAAUGGAGGUGU | 12102 | CCTGGAGAGAAATGGAGGTGT |
| 6438 | CCUGGAUGUCUCUGCUGAUGA | 12103 | CCTGGATGTCTCTGCTGATGA |
| 6439 | CCUGGCACUCAGCGCUGCCAU | 12104 | CCTGGCACTCAGCGCTGCCAT |
| 6440 | CCUGGCCAGAGGCCAUGGGCU | 12105 | CCTGGCCAGAGGCCATGGGCT |
| 6441 | CCUGGCCAGCAGCAUGCCGUC | 12106 | CCTGGCCAGCAGCATGCCGTC |
| 6442 | CCUGGCCUGGGAUCCCAUCCC | 12107 | CCTGGCCTGGGATCCCATCCC |
| 6443 | CCUGGCUGUCUAGCAAGGCUU | 12108 | CCTGGCTGTCTAGCAAGGCTT |
| 6444 | CCUGGCUUAAGGAAGGUACAA | 12109 | CCTGGCTTAAGGAAGGTACAA |
| 6445 | CCUGGCUUCCCGCGAUUGCAC | 12110 | CCTGGCTTCCCGCGATTGCAC |
| 6446 | CCUGGGACUCAUCAGAGCCAU | 12111 | CCTGGGACTCATCAGAGCCAT |
| 6447 | CCUGGGAGCACGUCUUGGGGG | 12112 | CCTGGGAGCACGTCTTGGGGG |
| 6448 | CCUGGGAUCCCAUCCCAACAC | 12113 | CCTGGGATCCCATCCCAACAC |
| 6449 | CCUGGGCUCAGGCGAUCCUCC | 12114 | CCTGGGCTCAGGCGATCCTCC |
| 6450 | CCUGGGGACAAUUGCCAAUCC | 12115 | CCTGGGGACAATTGCCAATCC |
| 6451 | CCUGGGUGCAGGCCACGUGUC | 12116 | CCTGGGTGCAGGCCACGTGTC |
| 6452 | CCUGGGUGGCCACUGCAGCCU | 12117 | CCTGGGTGGCCACTGCAGCCT |
| 6453 | CCUGGUCCCCCCGGGCCUGUU | 12118 | CCTGGTCCCCCCGGGCCTGTT |
| 6454 | CCUGGUCUCCAACUCCUGAAC | 12119 | CCTGGTCTCCAACTCCTGAAC |
| 6455 | CCUGGUUGUGGCAAAUGUGGA | 12120 | CCTGGTTGTGGCAAATGTGGA |
| 6456 | CCUGUGAGGCGGUUGGCACUG | 12121 | CCTGTGAGGCGGTTGGCACTG |
| 6457 | CCUGUGUCUGGCCAAAUUCAU | 12122 | CCTGTGTCTGGCCAAATTCAT |
| 6458 | CCUGUUCUGCCUCCCAGAUGA | 12123 | CCTGTTCTGCCTCCCAGATGA |
| 6459 | CCUGUUUCUCUUAUCCUUCAC | 12124 | CCTGTTTCTCTTATCCTTCAC |
| 6460 | CCUUACGGCUGUGGAGCUGAC | 12125 | CCTTACGGCTGTGGAGCTGAC |
| 6461 | CCUUCACGAGGAAAGGAAGAA | 12126 | CCTTCACGAGGAAAGGAAGAA |
| 6462 | CCUUCCUCACACUGGCACUUG | 12127 | CCTTCCTCACACTGGCACTTG |
| 6463 | CCUUCGUGUGGGGGUCCAGCU | 12128 | CCTTCGTGTGGGGGTCCAGCT |
| 6464 | CCUUCUAUACAGUGGUUUAAA | 12129 | CCTTCTATACAGTGGTTTAAA |
| 6465 | CCUUGAGGGUGGCCGGGGGCC | 12130 | CCTTGAGGGTGGCCGGGGGCC |
| 6466 | CCUUGCAGGCCUUCGUGUGGG | 12131 | CCTTGCAGGCCTTCGTGTGGG |
| 6467 | CCUUGCAGUCAUAUUCCCGGU | 12132 | CCTTGCAGTCATATTCCCGGT |
| 6468 | CCUUGCAGUCGGGGCCACCAU | 12133 | CCTTGCAGTCGGGGCCACCAT |
| 6469 | CCUUGCUCGUCUGAGCCGUUG | 12134 | CCTTGCTCGTCTGAGCCGTTG |
| 6470 | CCUUGGAACACGUAAAGACCC | 12135 | CCTTGGAACACGTAAAGACCC |
| 6471 | CCUUGGCUUGGAGCCGUUCUC | 12136 | CCTTGGCTTGGAGCCGTTCTC |
| 6472 | CCUUGGUAUCCGCAACAGAGA | 12137 | CCTTGGTATCCGCAACAGAGA |
| 6473 | CCUUGUGACAUCUUCACGCGG | 12138 | CCTTGTGACATCTTCACGCGG |
| 6474 | CCUUUAGCCUGACGGUGGAUG | 12139 | CCTTTAGCCTGACGGTGGATG |
| 6475 | CCUUUCAUCUUCCAUGUUUGA | 12140 | CCTTTCATCTTCCATGTTTGA |
| 6476 | CCUUUCCUGGCUUAAGGAAGG | 12141 | CCTTTCCTGGCTTAAGGAAGG |
| 6477 | CCUUUCUUGAUCUUGGCGGGA | 12142 | CCTTTCTTGATCTTGGCGGGA |
| 6478 | CCUUUGUGACAGGAACCGCGG | 12143 | CCTTTGTGACAGGAACCGCGG |
| 6479 | CGAAACCAGAAAGGCUUAUCA | 12144 | CGAAACCAGAAAGGCTTATCA |
| 6480 | CGAAACUCGUCCUGGGAGCAC | 12145 | CGAAACTCGTCCTGGGAGCAC |
| 6481 | CGAAAGACACUGGGUGGGUCA | 12146 | CGAAAGACACTGGGTGGGTCA |
| 6482 | CGAACUGCCGAGAGAUGCACU | 12147 | CGAACTGCCGAGAGATGCACT |
| 6483 | CGAAGCCUGUUCUGCCUCCCA | 12148 | CGAAGCCTGTTCTGCCTCCCA |
| 6484 | CGAAGGCCGAGCAGGGGCUAC | 12149 | CGAAGGCCGAGCAGGGGCTAC |
| 6485 | CGACAAGAUUGGGGAAGUGAA | 12150 | CGACAAGATTGGGGAAGTGAA |
| 6486 | CGACAAUGGCAGUUCUCGAUA | 12151 | CGACAATGGCAGTTCTCGATA |
| 6487 | CGACAGAAAACAGUGCAAAAU | 12152 | CGACAGAAAACAGTGCAAAAT |
| 6488 | CGACAGCCUCAAAUGCCUGAG | 12153 | CGACAGCCTCAAATGCCTGAG |
| 6489 | CGACAGGUGGCCACAGCGCAG | 12154 | CGACAGGTGGCCACAGCGCAG |
| 6490 | CGACAGGUGGCCACAGGACAG | 12155 | CGACAGGTGGCCACAGGACAG |
| 6491 | CGACCACGUUCCUCAGGUUGG | 12156 | CGACCACGTTCCTCAGGTTGG |
| 6492 | CGACCCGGAUCACGACCUGCU | 12157 | CGACCCGGATCACGACCTGCT |
| 6493 | CGACCUGCUGUGUCCUAGCUG | 12158 | CGACCTGCTGTGTCCTAGCTG |
| 6494 | CGACGCCAUGAAUCCCUUUCC | 12159 | CGACGCCATGAATCCCTTTCC |
| 6495 | CGACGGUCCAGCGCAAUUUCC | 12160 | CGACGGTCCAGCGCAATTTCC |
| 6496 | CGACUCGUGUAUGUCGCGGAG | 12161 | CGACTCGTGTATGTCGCGGAG |
| 6497 | CGAGAAUAGUCACUGAACAAA | 12162 | CGAGAATAGTCACTGAACAAA |
| 6498 | CGAGACCAGAGACCCAGCUCC | 12163 | CGAGACCAGAGACCCAGCTCC |
| 6499 | CGAGAGAUGCACUUCCCAUCG | 12164 | CGAGAGATGCACTTCCCATCG |
| 6500 | CGAGCAGGGGCUACUGUCCCC | 12165 | CGAGCAGGGGCTACTGTCCCC |
| 6501 | CGAGCCAUCUUCGCAGUCGGG | 12166 | CGAGCCATCTTCGCAGTCGGG |
| 6502 | CGAGGAAAGGAAGAAACCAAA | 12167 | CGAGGAAAGGAAGAAACCAAA |
| 6503 | CGAGGAGCAAGGCGACGGUCC | 12168 | CGAGGAGCAAGGCGACGGTCC |
| 6504 | CGAGGAGCACGAUGGGGAGGA | 12169 | CGAGGAGCACGATGGGGAGGA |
| 6505 | CGAGGAGCACUGUAGGGAGAG | 12170 | CGAGGAGCACTGTAGGGAGAG |
| 6506 | CGAGGCCUUGAGGGUGGCCGG | 12171 | CGAGGCCTTGAGGGTGGCCGG |
| 6507 | CGAGGGCGCCCCCCAGGACAA | 12172 | CGAGGGCGCCCCCCAGGACAA |
| 6508 | CGAGGGGUAGCUGUAGCCGUC | 12173 | CGAGGGGTAGCTGTAGCCGTC |
| 6509 | CGAGGUCUCAGGAAGGGUUCU | 12174 | CGAGGTCTCAGGAAGGGTTCT |
| 6510 | CGAGUAGAUGUCCACACCAUU | 12175 | CGAGTAGATGTCCACACCATT |
| 6511 | CGAGUAGCUGGGACUACAGGU | 12176 | CGAGTAGCTGGGACTACAGGT |
| 6512 | CGAGUGGGGGUUGAUCUGCGG | 12177 | CGAGTGGGGGTTGATCTGCGG |
| 6513 | CGAUAUCGCACUCUUUGAUGG | 12178 | CGATATCGCACTCTTTGATGG |
| 6514 | CGAUAUCUUCGCAUCUUCGCU | 12179 | CGATATCTTCGCATCTTCGCT |
| 6515 | CGAUAUUCACGAUCACGGCUU | 12180 | CGATATTCACGATCACGGCTT |
| 6516 | CGAUCACGGCUUUGAAGUCCC | 12181 | CGATCACGGCTTTGAAGTCCC |
| 6517 | CGAUCAUGGCUCACUGCAACC | 12182 | CGATCATGGCTCACTGCAACC |
| 6518 | CGAUCCAGACUGGAGGAUUUA | 12183 | CGATCCAGACTGGAGGATTTA |
| 6519 | CGAUCCUCCUGCCUCAGCCUC | 12184 | CGATCCTCCTGCCTCAGCCTC |
| 6520 | CGAUCUCCUGACCUCAUGAUC | 12185 | CGATCTCCTGACCTCATGATC |
| 6521 | CGAUCUUAAGGUCAUUGCAGA | 12186 | CGATCTTAAGGTCATTGCAGA |
| 6522 | CGAUGCUUGAGAUGGAGUGAA | 12187 | CGATGCTTGAGATGGAGTGAA |
| 6523 | CGAUGGAGCCCACAGCCUUGC | 12188 | CGATGGAGCCCACAGCCTTGC |
| 6524 | CGAUGGCCCUUGGCUUGGAGC | 12189 | CGATGGCCCTTGGCTTGGAGC |
| 6525 | CGAUGGGGAGGACAAUGGACA | 12190 | CGATGGGGAGGACAATGGACA |
| 6526 | CGAUUUCAGAAAGAACCUGAA | 12191 | CGATTTCAGAAAGAACCTGAA |
| 6527 | CGCAACAGAGACAGUGCCCAG | 12192 | CGCAACAGAGACAGTGCCCAG |
| 6528 | CGCAAUCUCGGCUCACUGCAA | 12193 | CGCAATCTCGGCTCACTGCAA |
| 6529 | CGCAAUUUCCAGCCCCAGGGC | 12194 | CGCAATTTCCAGCCCCAGGGC |
| 6530 | CGCACCUCCAGAACUGAGGAA | 12195 | CGCACCTCCAGAACTGAGGAA |
| 6531 | CGCACUCUUUGAUGGGUUCAU | 12196 | CGCACTCTTTGATGGGTTCAT |
| 6532 | CGCAGACCCACUUGUAGGAGA | 12197 | CGCAGACCCACTTGTAGGAGA |
| 6533 | CGCAGAGCUGGCUGCAGGUGU | 12198 | CGCAGAGCTGGCTGCAGGTGT |
| 6534 | CGCAGAGUGUCACAUUAACGC | 12199 | CGCAGAGTGTCACATTAACGC |
| 6535 | CGCAGAGUGUCAGACAGCCUU | 12200 | CGCAGAGTGTCAGACAGCCTT |
| 6536 | CGCAGCCAACUUCAUCGCUCA | 12201 | CGCAGCCAACTTCATCGCTCA |
| 6537 | CGCAGCCUCUGCCAGGCAGUG | 12202 | CGCAGCCTCTGCCAGGCAGTG |
| 6538 | CGCAGGCCCACAGCUGGGGGA | 12203 | CGCAGGCCCACAGCTGGGGGA |
| 6539 | CGCAGGUAAACUUGGGCGAGU | 12204 | CGCAGGTAAACTTGGGCGAGT |
| 6540 | CGCAGUCCACUUGGCCAUCGC | 12205 | CGCAGTCCACTTGGCCATCGC |
| 6541 | CGCAGUCGGGGUCGUUGUCGC | 12206 | CGCAGTCGGGGTCGTTGTCGC |
| 6542 | CGCAGUUUUCCUCGUCAGAUU | 12207 | CGCAGTTTTCCTCGTCAGATT |
| 6543 | CGCAUCUGUCGCCCACGUGGC | 12208 | CGCATCTGTCGCCCACGTGGC |
| 6544 | CGCAUCUGUCGCCCACUGCAG | 12209 | CGCATCTGTCGCCCACTGCAG |
| 6545 | CGCAUCUUCGCUGGGCCACCA | 12210 | CGCATCTTCGCTGGGCCACCA |
| 6546 | CGCAUUUACGUGCUCCGAAAC | 12211 | CGCATTTACGTGCTCCGAAAC |
| 6547 | CGCCACAGAGCACAGCGGAAA | 12212 | CGCCACAGAGCACAGCGGAAA |
| 6548 | CGCCACCACGCCCGGCUAAUU | 12213 | CGCCACCACGCCCGGCTAATT |
| 6549 | CGCCACGUCAUCCUCCAGACU | 12214 | CGCCACGTCATCCTCCAGACT |
| 6550 | CGCCACUUAGGCAGUGGAACU | 12215 | CGCCACTTAGGCAGTGGAACT |
| 6551 | CGCCAGCUGGAGUGGAUGCAC | 12216 | CGCCAGCTGGAGTGGATGCAC |
| 6552 | CGCCAGGAGGGCCCAGGAUAA | 12217 | CGCCAGGAGGGCCCAGGATAA |
| 6553 | CGCCAGUUCUUCCAUAGAAGG | 12218 | CGCCAGTTCTTCCATAGAAGG |
| 6554 | CGCCAUGAAUCCCUUUCCUGG | 12219 | CGCCATGAATCCCTTTCCTGG |
| 6555 | CGCCAUGCCUGGCCAGAGGCC | 12220 | CGCCATGCCTGGCCAGAGGCC |
| 6556 | CGCCAUUGCUCAGGGUGGUCC | 12221 | CGCCATTGCTCAGGGTGGTCC |
| 6557 | CGCCCACGUGGCUGAAGAUCU | 12222 | CGCCCACGTGGCTGAAGATCT |
| 6558 | CGCCCACUGCAGUCCCCGCCG | 12223 | CGCCCACTGCAGTCCCCGCCG |
| 6559 | CGCCCAGAGCUUGGUGAGACA | 12224 | CGCCCAGAGCTTGGTGAGACA |
| 6560 | CGCCCAGGCCAGACUGCGGAC | 12225 | CGCCCAGGCCAGACTGCGGAC |
| 6561 | CGCCCCCCAGGACAAGGUGGG | 12226 | CGCCCCCCAGGACAAGGTGGG |
| 6562 | CGCCCGGCCUCACCGUGCAUG | 12227 | CGCCCGGCCTCACCGTGCATG |
| 6563 | CGCCCGGCUAAUUUUUUGUAU | 12228 | CGCCCGGCTAATTTTTTGTAT |
| 6564 | CGCCCUUGGUAUCCGCAACAG | 12229 | CGCCCTTGGTATCCGCAACAG |
| 6565 | CGCCGCGGCGAGGAGCAAGGC | 12230 | CGCCGCGGCGAGGAGCAAGGC |
| 6566 | CGCCGCUGUGACACUUGAACU | 12231 | CGCCGCTGTGACACTTGAACT |
| 6567 | CGCCGUGGGCUCUGUCAAGCU | 12232 | CGCCGTGGGCTCTGTCAAGCT |
| 6568 | CGCCGUUGUUGUCCAAGCAUU | 12233 | CGCCGTTGTTGTCCAAGCATT |
| 6569 | CGCCUCGGCCUCCCAAAGUGC | 12234 | CGCCTCGGCCTCCCAAAGTGC |
| 6570 | CGCCUUUGUGACAGGAACCGC | 12235 | CGCCTTTGTGACAGGAACCGC |
| 6571 | CGCGGAGGAACGCCAGGCCUC | 12236 | CGCGGAGGAACGCCAGGCCTC |
| 6572 | CGCGGAGGCGGCUCCGGGGAC | 12237 | CGCGGAGGCGGCTCCGGGGAC |
| 6573 | CGCGGCGAGGAGCAAGGCGAC | 12238 | CGCGGCGAGGAGCAAGGCGAC |
| 6574 | CGCGGGAGUCUGACGGAGGCU | 12239 | CGCGGGAGTCTGACGGAGGCT |
| 6575 | CGCUACUGGGCUUCUUCUCAU | 12240 | CGCTACTGGGCTTCTTCTCAT |
| 6576 | CGCUCAUGUCCUUGCAGUCAU | 12241 | CGCTCATGTCCTTGCAGTCAT |
| 6577 | CGCUCCGGUCCAGCGUCAUCU | 12242 | CGCTCCGGTCCAGCGTCATCT |
| 6578 | CGCUCUGUCACACAGCUGCCC | 12243 | CGCTCTGTCACACAGCTGCCC |
| 6579 | CGCUCUGUCGCCCAGGCCAGA | 12244 | CGCTCTGTCGCCCAGGCCAGA |
| 6580 | CGCUGCCAUCGCAGACCCACU | 12245 | CGCTGCCATCGCAGACCCACT |
| 6581 | CGCUGCGGCCACUCAUCCGAG | 12246 | CGCTGCGGCCACTCATCCGAG |
| 6582 | CGCUGGGCCACCAGCUGGAAG | 12247 | CGCTGGGCCACCAGCTGGAAG |
| 6583 | CGCUGUGACACUUGAACUUGU | 12248 | CGCTGTGACACTTGAACTTGT |
| 6584 | CGCUUCCCGGGUUCACACCAU | 12249 | CGCTTCCCGGGTTCACACCAT |
| 6585 | CGCUUUGUUGCUCAGGCCUGG | 12250 | CGCTTTGTTGCTCAGGCCTGG |
| 6586 | CGGAAACCGGGGACUUCGCCA | 12251 | CGGAAACCGGGGACTTCGCCA |
| 6587 | CGGAACCUGUGAGGCGGUUGG | 12252 | CGGAACCTGTGAGGCGGTTGG |
| 6588 | CGGACAUAAAGACGACAAUGG | 12253 | CGGACATAAAGACGACAATGG |
| 6589 | CGGACAUCAGUGCAACAGUAA | 12254 | CGGACATCAGTGCAACAGTAA |
| 6590 | CGGACUGCAGUGGCGCAAUCU | 12255 | CGGACTGCAGTGGCGCAATCT |
| 6591 | CGGAGGAACGCCAGGCCUCCC | 12256 | CGGAGGAACGCCAGGCCTCCC |
| 6592 | CGGAGGCGGCUCCGGGGACCC | 12257 | CGGAGGCGGCTCCGGGGACCC |
| 6593 | CGGAGGCUGUCACUGUCUCUC | 12258 | CGGAGGCTGTCACTGTCTCTC |
| 6594 | CGGAGGGCGACUCGUGUAUGU | 12259 | CGGAGGGCGACTCGTGTATGT |
| 6595 | CGGAGGUGUCGCCACAGAGCA | 12260 | CGGAGGTGTCGCCACAGAGCA |
| 6596 | CGGAGUCUCGCUCUGUCGCCC | 12261 | CGGAGTCTCGCTCTGTCGCCC |
| 6597 | CGGAUCACGACCUGCUGUGUC | 12262 | CGGATCACGACCTGCTGTGTC |
| 6598 | CGGAUUCAGCCAGAUCAUUUC | 12263 | CGGATTCAGCCAGATCATTTC |
| 6599 | CGGAUUUGCAGGUGACAGACA | 12264 | CGGATTTGCAGGTGACAGACA |
| 6600 | CGGCAGUCUCUAGCCAUGUUG | 12265 | CGGCAGTCTCTAGCCATGTTG |
| 6601 | CGGCAUUCAUUGACACGGGCU | 12266 | CGGCATTCATTGACACGGGCT |
| 6602 | CGGCCAAGGAGAAGGGGUGGG | 12267 | CGGCCAAGGAGAAGGGGTGGG |
| 6603 | CGGCCAAGGUAACCGGGUGUC | 12268 | CGGCCAAGGTAACCGGGTGTC |
| 6604 | CGGCCACUCAUCCGAGCCAUC | 12269 | CGGCCACTCATCCGAGCCATC |
| 6605 | CGGCCACUGAGGAGAUCUAGG | 12270 | CGGCCACTGAGGAGATCTAGG |
| 6606 | CGGCCCCCACAGCUGAAGUCC | 12271 | CGGCCCCCACAGCTGAAGTCC |
| 6607 | CGGCCUCACCGUGCAUGUUUU | 12272 | CGGCCTCACCGTGCATGTTTT |
| 6608 | CGGCCUCCCAAAGUGCUGGGA | 12273 | CGGCCTCCCAAAGTGCTGGGA |
| 6609 | CGGCGAGGAGCAAGGCGACGG | 12274 | CGGCGAGGAGCAAGGCGACGG |
| 6610 | CGGCGAGGUCUCAGGAAGGGU | 12275 | CGGCGAGGTCTCAGGAAGGGT |
| 6611 | CGGCGAUUUCAGAAAGAACCU | 12276 | CGGCGATTTCAGAAAGAACCT |
| 6612 | CGGCUAAUUUUUUGUAUUUUU | 12277 | CGGCTAATTTTTTGTATTTTT |
| 6613 | CGGCUCACUGCAAGCUCCGCU | 12278 | CGGCTCACTGCAAGCTCCGCT |
| 6614 | CGGCUCCGGGGACCCGGGACU | 12279 | CGGCTCCGGGGACCCGGGACT |
| 6615 | CGGCUGCAGACCAAGGAGGGG | 12280 | CGGCTGCAGACCAAGGAGGGG |
| 6616 | CGGCUGCCAUGGAUGCAGUUU | 12281 | CGGCTGCCATGGATGCAGTTT |
| 6617 | CGGCUGUGGAGCUGACCUUUA | 12282 | CGGCTGTGGAGCTGACCTTTA |
| 6618 | CGGCUUUGAAGUCCCGGUCAA | 12283 | CGGCTTTGAAGTCCCGGTCAA |
| 6619 | CGGGAACAGGUCGGGUGGUUG | 12284 | CGGGAACAGGTCGGGTGGTTG |
| 6620 | CGGGAAUGGAAGUGGGUAGGG | 12285 | CGGGAATGGAAGTGGGTAGGG |
| 6621 | CGGGACCACAGGUGAGCACCG | 12286 | CGGGACCACAGGTGAGCACCG |
| 6622 | CGGGACUCCAGGCAGAUGUUC | 12287 | CGGGACTCCAGGCAGATGTTC |
| 6623 | CGGGACUUGUCGUCGCCUUUG | 12288 | CGGGACTTGTCGTCGCCTTTG |
| 6624 | CGGGAGGAUGGGCACCAGGCU | 12289 | CGGGAGGATGGGCACCAGGCT |
| 6625 | CGGGAGGUGUCGGGAACAGGU | 12290 | CGGGAGGTGTCGGGAACAGGT |
| 6626 | CGGGAGUCUGACGGAGGCUGU | 12291 | CGGGAGTCTGACGGAGGCTGT |
| 6627 | CGGGAGUUCCCCAGUCAGUCC | 12292 | CGGGAGTTCCCCAGTCAGTCC |
| 6628 | CGGGAUCCUGACACUCAUCGA | 12293 | CGGGATCCTGACACTCATCGA |
| 6629 | CGGGCAGGAGGCCUCGUCUGA | 12294 | CGGGCAGGAGGCCTCGTCTGA |
| 6630 | CGGGCAGGCGCAGGUAAACUU | 12295 | CGGGCAGGCGCAGGTAAACTT |
| 6631 | CGGGCCUGUUUCUCUUAUCCU | 12296 | CGGGCCTGTTTCTCTTATCCT |
| 6632 | CGGGCUUUCCCUGGCCUGGGA | 12297 | CGGGCTTTCCCTGGCCTGGGA |
| 6633 | CGGGGACAUCAUUCUCCCAAA | 12298 | CGGGGACATCATTCTCCCAAA |
| 6634 | CGGGGACCCGGGACUUGUCGU | 12299 | CGGGGACCCGGGACTTGTCGT |
| 6635 | CGGGGACUUCGCCAGGAGGGC | 12300 | CGGGGACTTCGCCAGGAGGGC |
| 6636 | CGGGGAGAUUCUGAGGCAACG | 12301 | CGGGGAGATTCTGAGGCAACG |
| 6637 | CGGGGCACAGGCACUCGUAGC | 12302 | CGGGGCACAGGCACTCGTAGC |
| 6638 | CGGGGCAGGGAGGCACAGAUA | 12303 | CGGGGCAGGGAGGCACAGATA |
| 6639 | CGGGGCCACCAUCACAGCGCC | 12304 | CGGGGCCACCATCACAGCGCC |
| 6640 | CGGGGCUGGUGGGGCGAGGGC | 12305 | CGGGGCTGGTGGGGCGAGGGC |
| 6641 | CGGGGGCCCCUCUGUGAACUU | 12306 | CGGGGGCCCCTCTGTGAACTT |
| 6642 | CGGGGGCCUGGAUGUCUCUGC | 12307 | CGGGGGCCTGGATGTCTCTGC |
| 6643 | CGGGGGUGACAGGGCAAAGGC | 12308 | CGGGGGTGACAGGGCAAAGGC |
| 6644 | CGGGGUCGUUGUCGCAGGCCC | 12309 | CGGGGTCGTTGTCGCAGGCCC |
| 6645 | CGGGGUUGUCAAAGUUGAUGC | 12310 | CGGGGTTGTCAAAGTTGATGC |
| 6646 | CGGGGUUUCACGUGUUAGCCA | 12311 | CGGGGTTTCACGTGTTAGCCA |
| 6647 | CGGGUGGUUGUGUGCUGUGUC | 12312 | CGGGTGGTTGTGTGCTGTGTC |
| 6648 | CGGGUGUCUCAGGCACUUAAU | 12313 | CGGGTGTCTCAGGCACTTAAT |
| 6649 | CGGGUUCACACCAUUCUCCUG | 12314 | CGGGTTCACACCATTCTCCTG |
| 6650 | CGGUCAACCUGCCCCUCUCUG | 12315 | CGGTCAACCTGCCCCTCTCTG |
| 6651 | CGGUCACACUGCCGGCUGCCA | 12316 | CGGTCACACTGCCGGCTGCCA |
| 6652 | CGGUCCAGCGCAAUUUCCAGC | 12317 | CGGTCCAGCGCAATTTCCAGC |
| 6653 | CGGUCCAGCGUCAUCUUCCUG | 12318 | CGGTCCAGCGTCATCTTCCTG |
| 6654 | CGGUCCAGUAGAUGUUGCUGU | 12319 | CGGTCCAGTAGATGTTGCTGT |
| 6655 | CGGUCUGAGUCACAGACGAAC | 12320 | CGGTCTGAGTCACAGACGAAC |
| 6656 | CGGUGCCACCACGGAUUCAGC | 12321 | CGGTGCCACCACGGATTCAGC |
| 6657 | CGGUGGAGAGAAACUCAAAAC | 12322 | CGGTGGAGAGAAACTCAAAAC |
| 6658 | CGGUGGAUGUCUCCUGGGUGG | 12323 | CGGTGGATGTCTCCTGGGTGG |
| 6659 | CGGUGUCAUAGGAAGAGACGC | 12324 | CGGTGTCATAGGAAGAGACGC |
| 6660 | CGGUUCCCUGCACUGGGGGGG | 12325 | CGGTTCCCTGCACTGGGGGGG |
| 6661 | CGGUUGACACGGCCCCCACAG | 12326 | CGGTTGACACGGCCCCCACAG |
| 6662 | CGGUUGCCCCCGUUGACAUCG | 12327 | CGGTTGCCCCCGTTGACATCG |
| 6663 | CGGUUGGCACUGAAAAUGGCU | 12328 | CGGTTGGCACTGAAAATGGCT |
| 6664 | CGGUUGGUGAAGAAGAGGUAG | 12329 | CGGTTGGTGAAGAAGAGGTAG |
| 6665 | CGUAAAGACCCCUACAGCGCU | 12330 | CGTAAAGACCCCTACAGCGCT |
| 6666 | CGUAAGGAACCGAGGCCUUGA | 12331 | CGTAAGGAACCGAGGCCTTGA |
| 6667 | CGUACCCAGCUGAUUUUUAAC | 12332 | CGTACCCAGCTGATTTTTAAC |
| 6668 | CGUAGCCGAUCUUAAGGUCAU | 12333 | CGTAGCCGATCTTAAGGTCAT |
| 6669 | CGUCAAACGAUCCAGACUGGA | 12334 | CGTCAAACGATCCAGACTGGA |
| 6670 | CGUCAGAUUUGUCCUUGCAGU | 12335 | CGTCAGATTTGTCCTTGCAGT |
| 6671 | CGUCAGGGCGACAGGUGGCCA | 12336 | CGTCAGGGCGACAGGTGGCCA |
| 6672 | CGUCAUCCUCCAGACUGACCA | 12337 | CGTCATCCTCCAGACTGACCA |
| 6673 | CGUCAUCUUCCUGACCUCGUG | 12338 | CGTCATCTTCCTGACCTCGTG |
| 6674 | CGUCCAAGCAGUCCCGGUCUG | 12339 | CGTCCAAGCAGTCCCGGTCTG |
| 6675 | CGUCCGGGCAGGCGCAGGUAA | 12340 | CGTCCGGGCAGGCGCAGGTAA |
| 6676 | CGUCCUGGGAGCACGUCUUGG | 12341 | CGTCCTGGGAGCACGTCTTGG |
| 6677 | CGUCCUGGUUGUGGCAAAUGU | 12342 | CGTCCTGGTTGTGGCAAATGT |
| 6678 | CGUCGCCCAGAGCUUGGUGAG | 12343 | CGTCGCCCAGAGCTTGGTGAG |
| 6679 | CGUCGCCUUUGUGACAGGAAC | 12344 | CGTCGCCTTTGTGACAGGAAC |
| 6680 | CGUCGGGGCACAGGCACUCGU | 12345 | CGTCGGGGCACAGGCACTCGT |
| 6681 | CGUCGGGGGCCUGGAUGUCUC | 12346 | CGTCGGGGGCCTGGATGTCTC |
| 6682 | CGUCUCCUGGGACUCAUCAGA | 12347 | CGTCTCCTGGGACTCATCAGA |
| 6683 | CGUCUGAGCCGUCCAAGCAGU | 12348 | CGTCTGAGCCGTCCAAGCAGT |
| 6684 | CGUCUGAGCCGUUGUCGCAGU | 12349 | CGTCTGAGCCGTTGTCGCAGT |
| 6685 | CGUCUUGGCACUGGAACUCGU | 12350 | CGTCTTGGCACTGGAACTCGT |
| 6686 | CGUCUUGGGGGAGCACGUCUC | 12351 | CGTCTTGGGGGAGCACGTCTC |
| 6687 | CGUCUUGGGGGGACAGCCUUG | 12352 | CGTCTTGGGGGGACAGCCTTG |
| 6688 | CGUGAGCCACUGCGCCCGGCC | 12353 | CGTGAGCCACTGCGCCCGGCC |
| 6689 | CGUGAGGCACCUGUGUCUGGC | 12354 | CGTGAGGCACCTGTGTCTGGC |
| 6690 | CGUGCAAAUAAUCUUUGUACA | 12355 | CGTGCAAATAATCTTTGTACA |
| 6691 | CGUGCAUGUUUUAAACACAUA | 12356 | CGTGCATGTTTTAAACACATA |
| 6692 | CGUGCCACCGUACCCAGCUGA | 12357 | CGTGCCACCGTACCCAGCTGA |
| 6693 | CGUGCCGGUUGGUGAAGAAGA | 12358 | CGTGCCGGTTGGTGAAGAAGA |
| 6694 | CGUGCUCCGAAACCAGAAAGG | 12359 | CGTGCTCCGAAACCAGAAAGG |
| 6695 | CGUGGCAGCGAAACUCGUCCU | 12360 | CGTGGCAGCGAAACTCGTCCT |
| 6696 | CGUGGCGAUGGGGAGGACAAU | 12361 | CGTGGCGATGGGGAGGACAAT |
| 6697 | CGUGGCUGAAGAUCUCGGCUG | 12362 | CGTGGCTGAAGATCTCGGCTG |
| 6698 | CGUGGGAACAGCCGCCGUUGU | 12363 | CGTGGGAACAGCCGCCGTTGT |
| 6699 | CGUGGGCUCUGUCAAGCUGGG | 12364 | CGTGGGCTCTGTCAAGCTGGG |
| 6700 | CGUGGUGAGCCCAGGGGUGGC | 12365 | CGTGGTGAGCCCAGGGGTGGC |
| 6701 | CGUGUAUGUCGCGGAGGCGGC | 12366 | CGTGTATGTCGCGGAGGCGGC |
| 6702 | CGUGUCCAGAGCGACCACGUU | 12367 | CGTGTCCAGAGCGACCACGTT |
| 6703 | CGUGUCCUCACGGCCAAGGUA | 12368 | CGTGTCCTCACGGCCAAGGTA |
| 6704 | CGUGUGGGGGUCCAGCUGGAA | 12369 | CGTGTGGGGGTCCAGCTGGAA |
| 6705 | CGUGUUAGCCAGGAUGGUCUC | 12370 | CGTGTTAGCCAGGATGGTCTC |
| 6706 | CGUUCCUCAGGUUGGGGAUGA | 12371 | CGTTCCTCAGGTTGGGGATGA |
| 6707 | CGUUCUCCCUGAAUAACGUUU | 12372 | CGTTCTCCCTGAATAACGTTT |
| 6708 | CGUUGACAUCGAUGCUUGAGA | 12373 | CGTTGACATCGATGCTTGAGA |
| 6709 | CGUUGACUCCUGAGUUUAGGG | 12374 | CGTTGACTCCTGAGTTTAGGG |
| 6710 | CGUUGAUGAUAUCUGUCCAAA | 12375 | CGTTGATGATATCTGTCCAAA |
| 6711 | CGUUGCACACAGUCCAGUUCG | 12376 | CGTTGCACACAGTCCAGTTCG |
| 6712 | CGUUGGUCCCGCACUCUUUGA | 12377 | CGTTGGTCCCGCACTCTTTGA |
| 6713 | CGUUGGUGCCAUCUGCUGUUG | 12378 | CGTTGGTGCCATCTGCTGTTG |
| 6714 | CGUUGUCGCAGGCCCACAGCU | 12379 | CGTTGTCGCAGGCCCACAGCT |
| 6715 | CGUUGUCGCAGUCCACUUGGC | 12380 | CGTTGTCGCAGTCCACTTGGC |
| 6716 | CGUUGUUGUCCAAGCAUUCGU | 12381 | CGTTGTTGTCCAAGCATTCGT |
| 6717 | CGUUUCAACCUCGUAAGGAAC | 12382 | CGTTTCAACCTCGTAAGGAAC |
| 6718 | CGUUUCUUUCGCAUCUGUCGC | 12383 | CGTTTCTTTCGCATCTGTCGC |
| 6719 | CGUUUUCCUCUUCACGCCCUU | 12384 | CGTTTTCCTCTTCACGCCCTT |
| 6720 | CUAACCUGGCUGUCUAGCAAG | 12385 | CTAACCTGGCTGTCTAGCAAG |
| 6721 | CUAAUUUUUUGUAUUUUUAGU | 12386 | CTAATTTTTTGTATTTTTAGT |
| 6722 | CUACAAAAACCUUCUAUACAG | 12387 | CTACAAAAACCTTCTATACAG |
| 6723 | CUACACAGGGCCCCGAGAAUA | 12388 | CTACACAGGGCCCCGAGAATA |
| 6724 | CUACAGCGCUGCGGCCACUCA | 12389 | CTACAGCGCTGCGGCCACTCA |
| 6725 | CUACAGGUGCCCGCCACCACG | 12390 | CTACAGGTGCCCGCCACCACG |
| 6726 | CUACUGGGCUUCUUCUCAUUU | 12391 | CTACTGGGCTTCTTCTCATTT |
| 6727 | CUACUGUCCCCUUGGAACACG | 12392 | CTACTGTCCCCTTGGAACACG |
| 6728 | CUAGCAAGGCUUGCAUAGAAG | 12393 | CTAGCAAGGCTTGCATAGAAG |
| 6729 | CUAGCCAUGUUGCAGACUUUG | 12394 | CTAGCCATGTTGCAGACTTTG |
| 6730 | CUAGCUGGAAACCCUGGCUUC | 12395 | CTAGCTGGAAACCCTGGCTTC |
| 6731 | CUAGGGUGAUGCCAUUGGGCC | 12396 | CTAGGGTGATGCCATTGGGCC |
| 6732 | CUAGGUGGGCGGACAUAAAGA | 12397 | CTAGGTGGGCGGACATAAAGA |
| 6733 | CUAUACAGUGGUUUAAAAAGU | 12398 | CTATACAGTGGTTTAAAAAGT |
| 6734 | CUAUCUCCACCGUGGUGAGCC | 12399 | CTATCTCCACCGTGGTGAGCC |
| 6735 | CUAUCUGUACAGGGACGCAUU | 12400 | CTATCTGTACAGGGACGCATT |
| 6736 | CUAUGUUGCCCAGGCUGGAAA | 12401 | CTATGTTGCCCAGGCTGGAAA |
| 6737 | CUAUUGCUGGCCACCUCCGUG | 12402 | CTATTGCTGGCCACCTCCGTG |
| 6738 | CUCAAAACUUCCUGGAGAGAA | 12403 | CTCAAAACTTCCTGGAGAGAA |
| 6739 | CUCAAACCAUCCUCCCACCUC | 12404 | CTCAAACCATCCTCCCACCTC |
| 6740 | CUCAAAGACGGCCAAGGAGAA | 12405 | CTCAAAGACGGCCAAGGAGAA |
| 6741 | CUCAAAUGCCUGAGCUCAAAC | 12406 | CTCAAATGCCTGAGCTCAAAC |
| 6742 | CUCAAGUGAUCCAGCUCAGCC | 12407 | CTCAAGTGATCCAGCTCAGCC |
| 6743 | CUCACACCAGUUCACUCCUCU | 12408 | CTCACACCAGTTCACTCCTCT |
| 6744 | CUCACACUGGCACUUGUAGCC | 12409 | CTCACACTGGCACTTGTAGCC |
| 6745 | CUCACCGUGCAUGUUUUAAAC | 12410 | CTCACCGTGCATGTTTTAAAC |
| 6746 | CUCACGACAGCCUCAAAUGCC | 12411 | CTCACGACAGCCTCAAATGCC |
| 6747 | CUCACGCUACUGGGCUUCUUC | 12412 | CTCACGCTACTGGGCTTCTTC |
| 6748 | CUCACGGCCAAGGUAACCGGG | 12413 | CTCACGGCCAAGGTAACCGGG |
| 6749 | CUCACUGCAACCUCCACCUCC | 12414 | CTCACTGCAACCTCCACCTCC |
| 6750 | CUCACUGCAAGCUCCGCUUCC | 12415 | CTCACTGCAAGCTCCGCTTCC |
| 6751 | CUCAGCCUCCCAAAGUGCUGG | 12416 | CTCAGCCTCCCAAAGTGCTGG |
| 6752 | CUCAGCCUCCCAAGUAGCUGG | 12417 | CTCAGCCTCCCAAGTAGCTGG |
| 6753 | CUCAGCCUCCCGAGUAGCUGG | 12418 | CTCAGCCTCCCGAGTAGCTGG |
| 6754 | CUCAGCCUCGCCAUGCCUGGC | 12419 | CTCAGCCTCGCCATGCCTGGC |
| 6755 | CUCAGCCUCUCUUGGCUGGGU | 12420 | CTCAGCCTCTCTTGGCTGGGT |
| 6756 | CUCAGCCUCUGUGAGGCAGCU | 12421 | CTCAGCCTCTGTGAGGCAGCT |
| 6757 | CUCAGCGCUGCCAUCGCAGAC | 12422 | CTCAGCGCTGCCATCGCAGAC |
| 6758 | CUCAGGAAGGGUUCUGGGCAG | 12423 | CTCAGGAAGGGTTCTGGGCAG |
| 6759 | CUCAGGCACUUAAUAAAUAUU | 12424 | CTCAGGCACTTAATAAATATT |
| 6760 | CUCAGGCCUGGUCUCCAACUC | 12425 | CTCAGGCCTGGTCTCCAACTC |
| 6761 | CUCAGGCGAUCCUCCUGCCUC | 12426 | CTCAGGCGATCCTCCTGCCTC |
| 6762 | CUCAGGCUCUGCCUCUGGGAA | 12427 | CTCAGGCTCTGCCTCTGGGAA |
| 6763 | CUCAGGGUGGUCCUCUCACAC | 12428 | CTCAGGGTGGTCCTCTCACAC |
| 6764 | CUCAGGUUGGGGAUGAGGCUG | 12429 | CTCAGGTTGGGGATGAGGCTG |
| 6765 | CUCAUACAUACAACGGGGACA | 12430 | CTCATACATACAACGGGGACA |
| 6766 | CUCAUCAGAGCCAUCCUGGCA | 12431 | CTCATCAGAGCCATCCTGGCA |
| 6767 | CUCAUCCGAGCCAUCUUCGCA | 12432 | CTCATCCGAGCCATCTTCGCA |
| 6768 | CUCAUCCUCUGUGGUCUUCUG | 12433 | CTCATCCTCTGTGGTCTTCTG |
| 6769 | CUCAUCGAUAUCGCACUCUUU | 12434 | CTCATCGATATCGCACTCTTT |
| 6770 | CUCAUCGAUAUCUUCGCAUCU | 12435 | CTCATCGATATCTTCGCATCT |
| 6771 | CUCAUGAUCCACCCGCCUCGG | 12436 | CTCATGATCCACCCGCCTCGG |
| 6772 | CUCAUGUCCCUGGCCAGCAGC | 12437 | CTCATGTCCCTGGCCAGCAGC |
| 6773 | CUCAUGUCCUUGCAGUCAUAU | 12438 | CTCATGTCCTTGCAGTCATAT |
| 6774 | CUCAUUUCCUCUGCCAGCAAC | 12439 | CTCATTTCCTCTGCCAGCAAC |
| 6775 | CUCCAACUCCUGAACUCAAGU | 12440 | CTCCAACTCCTGAACTCAAGT |
| 6776 | CUCCAAGAUGGUCUUCCGGUU | 12441 | CTCCAAGATGGTCTTCCGGTT |
| 6777 | CUCCACCGUGGUGAGCCCAGG | 12442 | CTCCACCGTGGTGAGCCCAGG |
| 6778 | CUCCACCUCCUGGGCUCAGGC | 12443 | CTCCACCTCCTGGGCTCAGGC |
| 6779 | CUCCAGAACUGAGGAAUGCAG | 12444 | CTCCAGAACTGAGGAATGCAG |
| 6780 | CUCCAGACUGACCAUCGAGGG | 12445 | CTCCAGACTGACCATCGAGGG |
| 6781 | CUCCAGACUGACCAUCUGUCU | 12446 | CTCCAGACTGACCATCTGTCT |
| 6782 | CUCCAGGCAGAUGUUCACGCC | 12447 | CTCCAGGCAGATGTTCACGCC |
| 6783 | CUCCAGGUUCACGCAGAGCUG | 12448 | CTCCAGGTTCACGCAGAGCTG |
| 6784 | CUCCCAAAAAGCGUUGCACAC | 12449 | CTCCCAAAAAGCGTTGCACAC |
| 6785 | CUCCCAAAGUGCUGGGAUUAC | 12450 | CTCCCAAAGTGCTGGGATTAC |
| 6786 | CUCCCAAGUAGCUGGGAUCAC | 12451 | CTCCCAAGTAGCTGGGATCAC |
| 6787 | CUCCCACCUCAGCCUCGCCAU | 12452 | CTCCCACCTCAGCCTCGCCAT |
| 6788 | CUCCCAGAAGCCACUCAUACA | 12453 | CTCCCAGAAGCCACTCATACA |
| 6789 | CUCCCAGAUGAAUAAAUAUAU | 12454 | CTCCCAGATGAATAAATATAT |
| 6790 | CUCCCCUUUCAUCUUCCAUGU | 12455 | CTCCCCTTTCATCTTCCATGT |
| 6791 | CUCCCGAGUAGCUGGGACUAC | 12456 | CTCCCGAGTAGCTGGGACTAC |
| 6792 | CUCCCUGAAUAACGUUUUCCU | 12457 | CTCCCTGAATAACGTTTTCCT |
| 6793 | CUCCGAAACCAGAAAGGCUUA | 12458 | CTCCGAAACCAGAAAGGCTTA |
| 6794 | CUCCGCUUCCCGGGUUCACAC | 12459 | CTCCGCTTCCCGGGTTCACAC |
| 6795 | CUCCGGACAUCAGUGCAACAG | 12460 | CTCCGGACATCAGTGCAACAG |
| 6796 | CUCCGGGGACCCGGGACUUGU | 12461 | CTCCGGGGACCCGGGACTTGT |
| 6797 | CUCCGGUCCAGCGUCAUCUUC | 12462 | CTCCGGTCCAGCGTCATCTTC |
| 6798 | CUCCGUGGCGAUGGGGAGGAC | 12463 | CTCCGTGGCGATGGGGAGGAC |
| 6799 | CUCCGUGUCCAGAGCGACCAC | 12464 | CTCCGTGTCCAGAGCGACCAC |
| 6800 | CUCCUCAUGUCCCUGGCCAGC | 12465 | CTCCTCATGTCCCTGGCCAGC |
| 6801 | CUCCUCUUGGCUGGGUGAGGU | 12466 | CTCCTCTTGGCTGGGTGAGGT |
| 6802 | CUCCUGAACUCAAGUGAUCCA | 12467 | CTCCTGAACTCAAGTGATCCA |
| 6803 | CUCCUGACCUCAUGAUCCACC | 12468 | CTCCTGACCTCATGATCCACC |
| 6804 | CUCCUGAGUUUAGGGGUUUGG | 12469 | CTCCTGAGTTTAGGGGTTTGG |
| 6805 | CUCCUGCCUCAGCCUCCCAAG | 12470 | CTCCTGCCTCAGCCTCCCAAG |
| 6806 | CUCCUGCCUCAGCCUCCCGAG | 12471 | CTCCTGCCTCAGCCTCCCGAG |
| 6807 | CUCCUGGGACUCAUCAGAGCC | 12472 | CTCCTGGGACTCATCAGAGCC |
| 6808 | CUCCUGGGCUCAGGCGAUCCU | 12473 | CTCCTGGGCTCAGGCGATCCT |
| 6809 | CUCCUGGGUGGCCACUGCAGC | 12474 | CTCCTGGGTGGCCACTGCAGC |
| 6810 | CUCGAAAGACACUGGGUGGGU | 12475 | CTCGAAAGACACTGGGTGGGT |
| 6811 | CUCGAAGGCCGAGCAGGGGCU | 12476 | CTCGAAGGCCGAGCAGGGGCT |
| 6812 | CUCGAGGGGUAGCUGUAGCCG | 12477 | CTCGAGGGGTAGCTGTAGCCG |
| 6813 | CUCGAUAUUCACGAUCACGGC | 12478 | CTCGATATTCACGATCACGGC |
| 6814 | CUCGAUCAUGGCUCACUGCAA | 12479 | CTCGATCATGGCTCACTGCAA |
| 6815 | CUCGAUCUCCUGACCUCAUGA | 12480 | CTCGATCTCCTGACCTCATGA |
| 6816 | CUCGCAGAGUGUCACAUUAAC | 12481 | CTCGCAGAGTGTCACATTAAC |
| 6817 | CUCGCAGAGUGUCAGACAGCC | 12482 | CTCGCAGAGTGTCAGACAGCC |
| 6818 | CUCGCAGCCUCUGCCAGGCAG | 12483 | CTCGCAGCCTCTGCCAGGCAG |
| 6819 | CUCGCCACUUAGGCAGUGGAA | 12484 | CTCGCCACTTAGGCAGTGGAA |
| 6820 | CUCGCCAUGCCUGGCCAGAGG | 12485 | CTCGCCATGCCTGGCCAGAGG |
| 6821 | CUCGCUCCGGUCCAGCGUCAU | 12486 | CTCGCTCCGGTCCAGCGTCAT |
| 6822 | CUCGCUCUGUCGCCCAGGCCA | 12487 | CTCGCTCTGTCGCCCAGGCCA |
| 6823 | CUCGCUUUGUUGCUCAGGCCU | 12488 | CTCGCTTTGTTGCTCAGGCCT |
| 6824 | CUCGGCCUCCCAAAGUGCUGG | 12489 | CTCGGCCTCCCAAAGTGCTGG |
| 6825 | CUCGGCUCACUGCAAGCUCCG | 12490 | CTCGGCTCACTGCAAGCTCCG |
| 6826 | CUCGGCUGCAGACCAAGGAGG | 12491 | CTCGGCTGCAGACCAAGGAGG |
| 6827 | CUCGGUGCCACCACGGAUUCA | 12492 | CTCGGTGCCACCACGGATTCA |
| 6828 | CUCGUAAGGAACCGAGGCCUU | 12493 | CTCGTAAGGAACCGAGGCCTT |
| 6829 | CUCGUAGCCGAUCUUAAGGUC | 12494 | CTCGTAGCCGATCTTAAGGTC |
| 6830 | CUCGUCAGAUUUGUCCUUGCA | 12495 | CTCGTCAGATTTGTCCTTGCA |
| 6831 | CUCGUCCUGGGAGCACGUCUU | 12496 | CTCGTCCTGGGAGCACGTCTT |
| 6832 | CUCGUCUGAGCCGUCCAAGCA | 12497 | CTCGTCTGAGCCGTCCAAGCA |
| 6833 | CUCGUCUGAGCCGUUGUCGCA | 12498 | CTCGTCTGAGCCGTTGTCGCA |
| 6834 | CUCGUGCCGGUUGGUGAAGAA | 12499 | CTCGTGCCGGTTGGTGAAGAA |
| 6835 | CUCGUGUAUGUCGCGGAGGCG | 12500 | CTCGTGTATGTCGCGGAGGCG |
| 6836 | CUCGUUUCUUUCGCAUCUGUC | 12501 | CTCGTTTCTTTCGCATCTGTC |
| 6837 | CUCUAGCCAUGUUGCAGACUU | 12502 | CTCTAGCCATGTTGCAGACTT |
| 6838 | CUCUCACACCAGUUCACUCCU | 12503 | CTCTCACACCAGTTCACTCCT |
| 6839 | CUCUCCGGACAUCAGUGCAAC | 12504 | CTCTCCGGACATCAGTGCAAC |
| 6840 | CUCUCUGUCCCCGGCAUUCAU | 12505 | CTCTCTGTCCCCGGCATTCAT |
| 6841 | CUCUCUUGGCUGGGUGAGGUU | 12506 | CTCTCTTGGCTGGGTGAGGTT |
| 6842 | CUCUGAACUGAGAAAGUGCAA | 12507 | CTCTGAACTGAGAAAGTGCAA |
| 6843 | CUCUGCCAGCAACGUCGCCCA | 12508 | CTCTGCCAGCAACGTCGCCCA |
| 6844 | CUCUGCCAGGCAGUGUCCCGA | 12509 | CTCTGCCAGGCAGTGTCCCGA |
| 6845 | CUCUGCCUCUGGGAAGUGGCA | 12510 | CTCTGCCTCTGGGAAGTGGCA |
| 6846 | CUCUGCUGAUGACGGUGUCAU | 12511 | CTCTGCTGATGACGGTGTCAT |
| 6847 | CUCUGGCAGGCAAUGCUUUGG | 12512 | CTCTGGCAGGCAATGCTTTGG |
| 6848 | CUCUGGGAAGUGGCAUCAUUU | 12513 | CTCTGGGAAGTGGCATCATTT |
| 6849 | CUCUGGGACAGGUCAGACCAG | 12514 | CTCTGGGACAGGTCAGACCAG |
| 6850 | CUCUGGGCCUGACAUCCCCUC | 12515 | CTCTGGGCCTGACATCCCCTC |
| 6851 | CUCUGGGGACAGUAGGUUUUC | 12516 | CTCTGGGGACAGTAGGTTTTC |
| 6852 | CUCUGUCAAGCUGGGUGCUGC | 12517 | CTCTGTCAAGCTGGGTGCTGC |
| 6853 | CUCUGUCACACAGCUGCCCCC | 12518 | CTCTGTCACACAGCTGCCCCC |
| 6854 | CUCUGUCCCCGGCAUUCAUUG | 12519 | CTCTGTCCCCGGCATTCATTG |
| 6855 | CUCUGUCGCCCAGGCCAGACU | 12520 | CTCTGTCGCCCAGGCCAGACT |
| 6856 | CUCUGUCUUUGAAUAAAACAA | 12521 | CTCTGTCTTTGAATAAAACAA |
| 6857 | CUCUGUGAACUUGAUCGGGGC | 12522 | CTCTGTGAACTTGATCGGGGC |
| 6858 | CUCUGUGAGGCAGCUCCUCAU | 12523 | CTCTGTGAGGCAGCTCCTCAT |
| 6859 | CUCUGUGGUCUUCUGAUAGAC | 12524 | CTCTGTGGTCTTCTGATAGAC |
| 6860 | CUCUUAUCCUUCACGAGGAAA | 12525 | CTCTTATCCTTCACGAGGAAA |
| 6861 | CUCUUCACGCCCUUGGUAUCC | 12526 | CTCTTCACGCCCTTGGTATCC |
| 6862 | CUCUUGGCUGGGUGAGGUUGU | 12527 | CTCTTGGCTGGGTGAGGTTGT |
| 6863 | CUCUUUGAUGGGUUCAUCUGA | 12528 | CTCTTTGATGGGTTCATCTGA |
| 6864 | CUCUUUUCAUCCUCCAAGAUG | 12529 | CTCTTTTCATCCTCCAAGATG |
| 6865 | CUGAAAAUGGCUUCGUUGAUG | 12530 | CTGAAAATGGCTTCGTTGATG |
| 6866 | CUGAAAUAGGGUCUUCAUCUG | 12531 | CTGAAATAGGGTCTTCATCTG |
| 6867 | CUGAACAAAUACAGCAACCAG | 12532 | CTGAACAAATACAGCAACCAG |
| 6868 | CUGAACUCAAGUGAUCCAGCU | 12533 | CTGAACTCAAGTGATCCAGCT |
| 6869 | CUGAACUGAGAAAGUGCAAGG | 12534 | CTGAACTGAGAAAGTGCAAGG |
| 6870 | CUGAAGAUCUCGGCUGCAGAC | 12535 | CTGAAGATCTCGGCTGCAGAC |
| 6871 | CUGAAGUCCCCGGAUUUGCAG | 12536 | CTGAAGTCCCCGGATTTGCAG |
| 6872 | CUGAAGUCCCGUCAAACGAUC | 12537 | CTGAAGTCCCGTCAAACGATC |
| 6873 | CUGAAUAACGUUUUCCUCUUC | 12538 | CTGAATAACGTTTTCCTCTTC |
| 6874 | CUGAAUGUUUUCAGUCACCAG | 12539 | CTGAATGTTTTCAGTCACCAG |
| 6875 | CUGACACUCAUCGAUAUCGCA | 12540 | CTGACACTCATCGATATCGCA |
| 6876 | CUGACACUCAUCGAUAUCUUC | 12541 | CTGACACTCATCGATATCTTC |
| 6877 | CUGACAGCCACACCUGGGUGC | 12542 | CTGACAGCCACACCTGGGTGC |
| 6878 | CUGACAUCCCCUCCCCUUUCA | 12543 | CTGACATCCCCTCCCCTTTCA |
| 6879 | CUGACCAGUCCCGGCAGUCUC | 12544 | CTGACCAGTCCCGGCAGTCTC |
| 6880 | CUGACCAUCGAGGGGUAGCUG | 12545 | CTGACCATCGAGGGGTAGCTG |
| 6881 | CUGACCAUCUGUCUCGAGGGG | 12546 | CTGACCATCTGTCTCGAGGGG |
| 6882 | CUGACCUCAUGAUCCACCCGC | 12547 | CTGACCTCATGATCCACCCGC |
| 6883 | CUGACCUCGUGCCGGUUGGUG | 12548 | CTGACCTCGTGCCGGTTGGTG |
| 6884 | CUGACCUUUAGCCUGACGGUG | 12549 | CTGACCTTTAGCCTGACGGTG |
| 6885 | CUGACGGAGGCUGUCACUGUC | 12550 | CTGACGGAGGCTGTCACTGTC |
| 6886 | CUGACGGUGGAUGUCUCCUGG | 12551 | CTGACGGTGGATGTCTCCTGG |
| 6887 | CUGAGAAAGUGCAAGGAGACC | 12552 | CTGAGAAAGTGCAAGGAGACC |
| 6888 | CUGAGCCGUCCAAGCAGUCCC | 12553 | CTGAGCCGTCCAAGCAGTCCC |
| 6889 | CUGAGCCGUUGUCGCAGUCCA | 12554 | CTGAGCCGTTGTCGCAGTCCA |
| 6890 | CUGAGCUCAAACCAUCCUCCC | 12555 | CTGAGCTCAAACCATCCTCCC |
| 6891 | CUGAGGAAUGCAGCGGUUGAC | 12556 | CTGAGGAATGCAGCGGTTGAC |
| 6892 | CUGAGGAGAUCUAGGGUGAUG | 12557 | CTGAGGAGATCTAGGGTGATG |
| 6893 | CUGAGGCAACGUUUCAACCUC | 12558 | CTGAGGCAACGTTTCAACCTC |
| 6894 | CUGAGUCACAGACGAACUGCC | 12559 | CTGAGTCACAGACGAACTGCC |
| 6895 | CUGAGUUUAGGGGUUUGGCUU | 12560 | CTGAGTTTAGGGGTTTGGCTT |
| 6896 | CUGAUAGACGGGGUUGUCAAA | 12561 | CTGATAGACGGGGTTGTCAAA |
| 6897 | CUGAUGACGGUGUCAUAGGAA | 12562 | CTGATGACGGTGTCATAGGAA |
| 6898 | CUGAUGCACCAUGCAUGGUUU | 12563 | CTGATGCACCATGCATGGTTT |
| 6899 | CUGAUUUUUAACUUUUUGGUU | 12564 | CTGATTTTTAACTTTTTGGTT |
| 6900 | CUGCAACCUCCACCUCCUGGG | 12565 | CTGCAACCTCCACCTCCTGGG |
| 6901 | CUGCAAGCUCCGCUUCCCGGG | 12566 | CTGCAAGCTCCGCTTCCCGGG |
| 6902 | CUGCACUGGGGGGGACACAGU | 12567 | CTGCACTGGGGGGGACACAGT |
| 6903 | CUGCAGACCAAGGAGGGGCGG | 12568 | CTGCAGACCAAGGAGGGGCGG |
| 6904 | CUGCAGAUCAUUCUCUGGGAC | 12569 | CTGCAGATCATTCTCTGGGAC |
| 6905 | CUGCAGCCUCAGCCUCUCUUG | 12570 | CTGCAGCCTCAGCCTCTCTTG |
| 6906 | CUGCAGCCUCAGCCUCUGUGA | 12571 | CTGCAGCCTCAGCCTCTGTGA |
| 6907 | CUGCAGGUGUCGGGAUCCUGA | 12572 | CTGCAGGTGTCGGGATCCTGA |
| 6908 | CUGCAGUCCCCGCCGCGGCGA | 12573 | CTGCAGTCCCCGCCGCGGCGA |
| 6909 | CUGCAGUGGCGCAAUCUCGGC | 12574 | CTGCAGTGGCGCAATCTCGGC |
| 6910 | CUGCCAGCAACGUCGCCCAGA | 12575 | CTGCCAGCAACGTCGCCCAGA |
| 6911 | CUGCCAGGCAGUGUCCCGACC | 12576 | CTGCCAGGCAGTGTCCCGACC |
| 6912 | CUGCCAUCGCAGACCCACUUG | 12577 | CTGCCATCGCAGACCCACTTG |
| 6913 | CUGCCAUGGAUGCAGUUUCCA | 12578 | CTGCCATGGATGCAGTTTCCA |
| 6914 | CUGCCCCCGAGACCAGAGACC | 12579 | CTGCCCCCGAGACCAGAGACC |
| 6915 | CUGCCCCUCUCUGUCCCCGGC | 12580 | CTGCCCCTCTCTGTCCCCGGC |
| 6916 | CUGCCGAGAGAUGCACUUCCC | 12581 | CTGCCGAGAGATGCACTTCCC |
| 6917 | CUGCCGGCUGCCAUGGAUGCA | 12582 | CTGCCGGCTGCCATGGATGCA |
| 6918 | CUGCCUCAGCCUCCCAAGUAG | 12583 | CTGCCTCAGCCTCCCAAGTAG |
| 6919 | CUGCCUCAGCCUCCCGAGUAG | 12584 | CTGCCTCAGCCTCCCGAGTAG |
| 6920 | CUGCCUCCCAGAUGAAUAAAU | 12585 | CTGCCTCCCAGATGAATAAAT |
| 6921 | CUGCCUCUGGGAAGUGGCAUC | 12586 | CTGCCTCTGGGAAGTGGCATC |
| 6922 | CUGCGCCCGGCCUCACCGUGC | 12587 | CTGCGCCCGGCCTCACCGTGC |
| 6923 | CUGCGGACUGCAGUGGCGCAA | 12588 | CTGCGGACTGCAGTGGCGCAA |
| 6924 | CUGCGGCCACUCAUCCGAGCC | 12589 | CTGCGGCCACTCATCCGAGCC |
| 6925 | CUGCGGGGCAGGGAGGCACAG | 12590 | CTGCGGGGCAGGGAGGCACAG |
| 6926 | CUGCUGAUGACGGUGUCAUAG | 12591 | CTGCTGATGACGGTGTCATAG |
| 6927 | CUGCUGAUGCACCAUGCAUGG | 12592 | CTGCTGATGCACCATGCATGG |
| 6928 | CUGCUGUGUCCUAGCUGGAAA | 12593 | CTGCTGTGTCCTAGCTGGAAA |
| 6929 | CUGCUGUUGUGUGUUGAAAGC | 12594 | CTGCTGTTGTGTGTTGAAAGC |
| 6930 | CUGGAAACCCUGGCUUCCCGC | 12595 | CTGGAAACCCTGGCTTCCCGC |
| 6931 | CUGGAAAGCAGUGGCAUAAUC | 12596 | CTGGAAAGCAGTGGCATAATC |
| 6932 | CUGGAACUCGUUUCUUUCGCA | 12597 | CTGGAACTCGTTTCTTTCGCA |
| 6933 | CUGGAAGCCGUCGGGGCACAG | 12598 | CTGGAAGCCGTCGGGGCACAG |
| 6934 | CUGGAAGCCUUCCUCACACUG | 12599 | CTGGAAGCCTTCCTCACACTG |
| 6935 | CUGGAAGCUGGCGGGACCACA | 12600 | CTGGAAGCTGGCGGGACCACA |
| 6936 | CUGGAAUUCGUCAGGGCGACA | 12601 | CTGGAATTCGTCAGGGCGACA |
| 6937 | CUGGAGAGAAAUGGAGGUGUC | 12602 | CTGGAGAGAAATGGAGGTGTC |
| 6938 | CUGGAGGAUUUAUUUAUUUUU | 12603 | CTGGAGGATTTATTTATTTTT |
| 6939 | CUGGAGUGCAGUGGCUCGAUC | 12604 | CTGGAGTGCAGTGGCTCGATC |
| 6940 | CUGGAGUGGAUGCACUCGCCA | 12605 | CTGGAGTGGATGCACTCGCCA |
| 6941 | CUGGAUGUCUCUGCUGAUGAC | 12606 | CTGGATGTCTCTGCTGATGAC |
| 6942 | CUGGCACUCAGCGCUGCCAUC | 12607 | CTGGCACTCAGCGCTGCCATC |
| 6943 | CUGGCACUUGUAGCCACCCUC | 12608 | CTGGCACTTGTAGCCACCCTC |
| 6944 | CUGGCAGCCGCCAUUGCUCAG | 12609 | CTGGCAGCCGCCATTGCTCAG |
| 6945 | CUGGCAGGCAAUGCUUUGGUC | 12610 | CTGGCAGGCAATGCTTTGGTC |
| 6946 | CUGGCCAAAUUCAUUUUUUAA | 12611 | CTGGCCAAATTCATTTTTTAA |
| 6947 | CUGGCCACCUCCGUGUCCAGA | 12612 | CTGGCCACCTCCGTGTCCAGA |
| 6948 | CUGGCCAGAGGCCAUGGGCUG | 12613 | CTGGCCAGAGGCCATGGGCTG |
| 6949 | CUGGCCAGCAGCAUGCCGUCC | 12614 | CTGGCCAGCAGCATGCCGTCC |
| 6950 | CUGGCCUGGGAUCCCAUCCCA | 12615 | CTGGCCTGGGATCCCATCCCA |
| 6951 | CUGGCGGGACCACAGGUGAGC | 12616 | CTGGCGGGACCACAGGTGAGC |
| 6952 | CUGGCUGCAGGUGUCGGGAUC | 12617 | CTGGCTGCAGGTGTCGGGATC |
| 6953 | CUGGCUGUCUAGCAAGGCUUG | 12618 | CTGGCTGTCTAGCAAGGCTTG |
| 6954 | CUGGCUUAAGGAAGGUACAAA | 12619 | CTGGCTTAAGGAAGGTACAAA |
| 6955 | CUGGCUUCCCGCGAUUGCACU | 12620 | CTGGCTTCCCGCGATTGCACT |
| 6956 | CUGGGAAGUGGCAUCAUUUGG | 12621 | CTGGGAAGTGGCATCATTTGG |
| 6957 | CUGGGACAGGUCAGACCAGUA | 12622 | CTGGGACAGGTCAGACCAGTA |
| 6958 | CUGGGACUACAGGUGCCCGCC | 12623 | CTGGGACTACAGGTGCCCGCC |
| 6959 | CUGGGACUCAUCAGAGCCAUC | 12624 | CTGGGACTCATCAGAGCCATC |
| 6960 | CUGGGAGCACGUCUUGGGGGA | 12625 | CTGGGAGCACGTCTTGGGGGA |
| 6961 | CUGGGAGCACGUCUUGGGGGG | 12626 | CTGGGAGCACGTCTTGGGGGG |
| 6962 | CUGGGAUCACAGGCACGUGCC | 12627 | CTGGGATCACAGGCACGTGCC |
| 6963 | CUGGGAUCCCAUCCCAACACA | 12628 | CTGGGATCCCATCCCAACACA |
| 6964 | CUGGGAUUACAGGCGUGAGCC | 12629 | CTGGGATTACAGGCGTGAGCC |
| 6965 | CUGGGAUUACAGGCGUGAGGC | 12630 | CTGGGATTACAGGCGTGAGGC |
| 6966 | CUGGGCAGGGACGGGACUCCA | 12631 | CTGGGCAGGGACGGGACTCCA |
| 6967 | CUGGGCCACCAGCUGGAAGCC | 12632 | CTGGGCCACCAGCTGGAAGCC |
| 6968 | CUGGGCCUGACAUCCCCUCCC | 12633 | CTGGGCCTGACATCCCCTCCC |
| 6969 | CUGGGCUCAGGCGAUCCUCCU | 12634 | CTGGGCTCAGGCGATCCTCCT |
| 6970 | CUGGGCUUCUUCUCAUUUCCU | 12635 | CTGGGCTTCTTCTCATTTCCT |
| 6971 | CUGGGGACAAUUGCCAAUCCC | 12636 | CTGGGGACAATTGCCAATCCC |
| 6972 | CUGGGGACAGUAGGUUUUCAG | 12637 | CTGGGGACAGTAGGTTTTCAG |
| 6973 | CUGGGGGAUGCAGGUGGAGCU | 12638 | CTGGGGGATGCAGGTGGAGCT |
| 6974 | CUGGGGGGGACACAGUGUUUU | 12639 | CTGGGGGGGACACAGTGTTTT |
| 6975 | CUGGGUGAGGUUGUGGAAGAG | 12640 | CTGGGTGAGGTTGTGGAAGAG |
| 6976 | CUGGGUGCAGGCCACGUGUCC | 12641 | CTGGGTGCAGGCCACGTGTCC |
| 6977 | CUGGGUGCUGCAGAUCAUUCU | 12642 | CTGGGTGCTGCAGATCATTCT |
| 6978 | CUGGGUGGCCACUGCAGCCUC | 12643 | CTGGGTGGCCACTGCAGCCTC |
| 6979 | CUGGGUGGGUCAUGAUUCGGG | 12644 | CTGGGTGGGTCATGATTCGGG |
| 6980 | CUGGUCCCCCCGGGCCUGUUU | 12645 | CTGGTCCCCCCGGGCCTGTTT |
| 6981 | CUGGUCUCCAACUCCUGAACU | 12646 | CTGGTCTCCAACTCCTGAACT |
| 6982 | CUGGUGGGGCGAGGGCGCCCC | 12647 | CTGGTGGGGCGAGGGCGCCCC |
| 6983 | CUGGUGUACUCGCUCCGGUCC | 12648 | CTGGTGTACTCGCTCCGGTCC |
| 6984 | CUGGUGUCCUGACAGCCACAC | 12649 | CTGGTGTCCTGACAGCCACAC |
| 6985 | CUGGUUGUGGCAAAUGUGGAC | 12650 | CTGGTTGTGGCAAATGTGGAC |
| 6986 | CUGUACAGGGACGCAUUUACG | 12651 | CTGTACAGGGACGCATTTACG |
| 6987 | CUGUAGCCGUCCUGGUUGUGG | 12652 | CTGTAGCCGTCCTGGTTGTGG |
| 6988 | CUGUAGGGAGAGGCACGCUCU | 12653 | CTGTAGGGAGAGGCACGCTCT |
| 6989 | CUGUCAAGCUGGGUGCUGCAG | 12654 | CTGTCAAGCTGGGTGCTGCAG |
| 6990 | CUGUCACACAGCUGCCCCCGA | 12655 | CTGTCACACAGCTGCCCCCGA |
| 6991 | CUGUCACUGUCUCUCCGGACA | 12656 | CTGTCACTGTCTCTCCGGACA |
| 6992 | CUGUCCAAAAUACUUUGUCCU | 12657 | CTGTCCAAAATACTTTGTCCT |
| 6993 | CUGUCCCCGGCAUUCAUUGAC | 12658 | CTGTCCCCGGCATTCATTGAC |
| 6994 | CUGUCCCCUUGGAACACGUAA | 12659 | CTGTCCCCTTGGAACACGTAA |
| 6995 | CUGUCCGAAGCCUGUUCUGCC | 12660 | CTGTCCGAAGCCTGTTCTGCC |
| 6996 | CUGUCGCCCACGUGGCUGAAG | 12661 | CTGTCGCCCACGTGGCTGAAG |
| 6997 | CUGUCGCCCACUGCAGUCCCC | 12662 | CTGTCGCCCACTGCAGTCCCC |
| 6998 | CUGUCGCCCAGGCCAGACUGC | 12663 | CTGTCGCCCAGGCCAGACTGC |
| 6999 | CUGUCUAGCAAGGCUUGCAUA | 12664 | CTGTCTAGCAAGGCTTGCATA |
| 7000 | CUGUCUCGAGGGGUAGCUGUA | 12665 | CTGTCTCGAGGGGTAGCTGTA |
| 7001 | CUGUCUCUCCGGACAUCAGUG | 12666 | CTGTCTCTCCGGACATCAGTG |
| 7002 | CUGUCUUUGAAUAAAACAAGG | 12667 | CTGTCTTTGAATAAAACAAGG |
| 7003 | CUGUGAACUUGAUCGGGGCUG | 12668 | CTGTGAACTTGATCGGGGCTG |
| 7004 | CUGUGACACUUGAACUUGUUG | 12669 | CTGTGACACTTGAACTTGTTG |
| 7005 | CUGUGAGGCAGCUCCUCAUGU | 12670 | CTGTGAGGCAGCTCCTCATGT |
| 7006 | CUGUGAGGCGGUUGGCACUGA | 12671 | CTGTGAGGCGGTTGGCACTGA |
| 7007 | CUGUGGAGCUGACCUUUAGCC | 12672 | CTGTGGAGCTGACCTTTAGCC |
| 7008 | CUGUGGAUCCAGUCCACAGCC | 12673 | CTGTGGATCCAGTCCACAGCC |
| 7009 | CUGUGGUCUUCUGAUAGACGG | 12674 | CTGTGGTCTTCTGATAGACGG |
| 7010 | CUGUGUCCUAGCUGGAAACCC | 12675 | CTGTGTCCTAGCTGGAAACCC |
| 7011 | CUGUGUCCUUACGGCUGUGGA | 12676 | CTGTGTCCTTACGGCTGTGGA |
| 7012 | CUGUGUCUGGCCAAAUUCAUU | 12677 | CTGTGTCTGGCCAAATTCATT |
| 7013 | CUGUUCUGCCUCCCAGAUGAA | 12678 | CTGTTCTGCCTCCCAGATGAA |
| 7014 | CUGUUGAUGUUCUUAAGCCGC | 12679 | CTGTTGATGTTCTTAAGCCGC |
| 7015 | CUGUUGCACUGGAAGCUGGCG | 12680 | CTGTTGCACTGGAAGCTGGCG |
| 7016 | CUGUUGCCCAGGCUGGAGUGC | 12681 | CTGTTGCCCAGGCTGGAGTGC |
| 7017 | CUGUUGUGUGUUGAAAGCCAC | 12682 | CTGTTGTGTGTTGAAAGCCAC |
| 7018 | CUGUUUCUCUUAUCCUUCACG | 12683 | CTGTTTCTCTTATCCTTCACG |
| 7019 | CUUAAGCCGCCAGUUCUUCCA | 12684 | CTTAAGCCGCCAGTTCTTCCA |
| 7020 | CUUAAGGAAGGUACAAACCCA | 12685 | CTTAAGGAAGGTACAAACCCA |
| 7021 | CUUAAGGUCAUUGCAGACGUG | 12686 | CTTAAGGTCATTGCAGACGTG |
| 7022 | CUUAAUAAAUAUUAAGGGUGA | 12687 | CTTAATAAATATTAAGGGTGA |
| 7023 | CUUACGGCUGUGGAGCUGACC | 12688 | CTTACGGCTGTGGAGCTGACC |
| 7024 | CUUACUAUGUUGCCCAGGCUG | 12689 | CTTACTATGTTGCCCAGGCTG |
| 7025 | CUUAGAGAUUGGUGGAUGAGG | 12690 | CTTAGAGATTGGTGGATGAGG |
| 7026 | CUUAGGCAGUGGAACUCGAAG | 12691 | CTTAGGCAGTGGAACTCGAAG |
| 7027 | CUUAUCACGGUUCCCUGCACU | 12692 | CTTATCACGGTTCCCTGCACT |
| 7028 | CUUAUCCUUCACGAGGAAAGG | 12693 | CTTATCCTTCACGAGGAAAGG |
| 7029 | CUUCACGAGGAAAGGAAGAAA | 12694 | CTTCACGAGGAAAGGAAGAAA |
| 7030 | CUUCACGCCCUUGGUAUCCGC | 12695 | CTTCACGCCCTTGGTATCCGC |
| 7031 | CUUCACGCGGGAGUCUGACGG | 12696 | CTTCACGCGGGAGTCTGACGG |
| 7032 | CUUCAUCGCUCAUGUCCUUGC | 12697 | CTTCATCGCTCATGTCCTTGC |
| 7033 | CUUCAUCUGUUGCCCAGGCUG | 12698 | CTTCATCTGTTGCCCAGGCTG |
| 7034 | CUUCCAUAGAAGGAAGACCCC | 12699 | CTTCCATAGAAGGAAGACCCC |
| 7035 | CUUCCAUGUUUGAGGAUUGUG | 12700 | CTTCCATGTTTGAGGATTGTG |
| 7036 | CUUCCCAUCGUGGCAGCGAAA | 12701 | CTTCCCATCGTGGCAGCGAAA |
| 7037 | CUUCCCGGGUUCACACCAUUC | 12702 | CTTCCCGGGTTCACACCATTC |
| 7038 | CUUCCGGUUGCCCCCGUUGAC | 12703 | CTTCCGGTTGCCCCCGTTGAC |
| 7039 | CUUCCUCACACUGGCACUUGU | 12704 | CTTCCTCACACTGGCACTTGT |
| 7040 | CUUCCUGACCUCGUGCCGGUU | 12705 | CTTCCTGACCTCGTGCCGGTT |
| 7041 | CUUCCUGGAGAGAAAUGGAGG | 12706 | CTTCCTGGAGAGAAATGGAGG |
| 7042 | CUUCGCAGUCGGGGUCGUUGU | 12707 | CTTCGCAGTCGGGGTCGTTGT |
| 7043 | CUUCGCAUCUUCGCUGGGCCA | 12708 | CTTCGCATCTTCGCTGGGCCA |
| 7044 | CUUCGCCAGGAGGGCCCAGGA | 12709 | CTTCGCCAGGAGGGCCCAGGA |
| 7045 | CUUCGCUGGGCCACCAGCUGG | 12710 | CTTCGCTGGGCCACCAGCTGG |
| 7046 | CUUCGUGUGGGGGUCCAGCUG | 12711 | CTTCGTGTGGGGGTCCAGCTG |
| 7047 | CUUCGUUGAUGAUAUCUGUCC | 12712 | CTTCGTTGATGATATCTGTCC |
| 7048 | CUUCUAUACAGUGGUUUAAAA | 12713 | CTTCTATACAGTGGTTTAAAA |
| 7049 | CUUCUCAUUUCCUCUGCCAGC | 12714 | CTTCTCATTTCCTCTGCCAGC |
| 7050 | CUUCUCUGGGCCUGACAUCCC | 12715 | CTTCTCTGGGCCTGACATCCC |
| 7051 | CUUCUCUGUCUUUGAAUAAAA | 12716 | CTTCTCTGTCTTTGAATAAAA |
| 7052 | CUUCUGAUAGACGGGGUUGUC | 12717 | CTTCTGATAGACGGGGTTGTC |
| 7053 | CUUCUUCUCAUUUCCUCUGCC | 12718 | CTTCTTCTCATTTCCTCTGCC |
| 7054 | CUUGAACUUGUUGGGUCCCUC | 12719 | CTTGAACTTGTTGGGTCCCTC |
| 7055 | CUUGAGAUGGAGUGAAGUUUG | 12720 | CTTGAGATGGAGTGAAGTTTG |
| 7056 | CUUGAGGGUGGCCGGGGGCCC | 12721 | CTTGAGGGTGGCCGGGGGCCC |
| 7057 | CUUGAUCGGGGCUGGUGGGGC | 12722 | CTTGATCGGGGCTGGTGGGGC |
| 7058 | CUUGAUCUUGGCGGGAGUUCC | 12723 | CTTGATCTTGGCGGGAGTTCC |
| 7059 | CUUGCAGGCCUUCGUGUGGGG | 12724 | CTTGCAGGCCTTCGTGTGGGG |
| 7060 | CUUGCAGUCAUAUUCCCGGUC | 12725 | CTTGCAGTCATATTCCCGGTC |
| 7061 | CUUGCAGUCGGGGCCACCAUC | 12726 | CTTGCAGTCGGGGCCACCATC |
| 7062 | CUUGCAUAGAAGAGGUAAACA | 12727 | CTTGCATAGAAGAGGTAAACA |
| 7063 | CUUGCUCGUCUGAGCCGUUGU | 12728 | CTTGCTCGTCTGAGCCGTTGT |
| 7064 | CUUGCUUCUCUGGGCCUGACA | 12729 | CTTGCTTCTCTGGGCCTGACA |
| 7065 | CUUGGAACACGUAAAGACCCC | 12730 | CTTGGAACACGTAAAGACCCC |
| 7066 | CUUGGAGCCGUUCUCCCUGAA | 12731 | CTTGGAGCCGTTCTCCCTGAA |
| 7067 | CUUGGAGGCAUUUGCAUAGAA | 12732 | CTTGGAGGCATTTGCATAGAA |
| 7068 | CUUGGCACUGGAACUCGUUUC | 12733 | CTTGGCACTGGAACTCGTTTC |
| 7069 | CUUGGCCAUCGCACCUCCAGA | 12734 | CTTGGCCATCGCACCTCCAGA |
| 7070 | CUUGGCGGGAGUUCCCCAGUC | 12735 | CTTGGCGGGAGTTCCCCAGTC |
| 7071 | CUUGGCUGGGUGAGGUUGUGG | 12736 | CTTGGCTGGGTGAGGTTGTGG |
| 7072 | CUUGGCUUGGAGCCGUUCUCC | 12737 | CTTGGCTTGGAGCCGTTCTCC |
| 7073 | CUUGGGCGAGUGGGGGUUGAU | 12738 | CTTGGGCGAGTGGGGGTTGAT |
| 7074 | CUUGGGGGAGCACGUCUCCUG | 12739 | CTTGGGGGAGCACGTCTCCTG |
| 7075 | CUUGGGGGGACAGCCUUGCUC | 12740 | CTTGGGGGGACAGCCTTGCTC |
| 7076 | CUUGGUAUCCGCAACAGAGAC | 12741 | CTTGGTATCCGCAACAGAGAC |
| 7077 | CUUGGUGAGACAUUGUCACUA | 12742 | CTTGGTGAGACATTGTCACTA |
| 7078 | CUUGUAGCCACCCUCCAGGUU | 12743 | CTTGTAGCCACCCTCCAGGTT |
| 7079 | CUUGUAGGAGAUGCAUUUCCC | 12744 | CTTGTAGGAGATGCATTTCCC |
| 7080 | CUUGUCGUCGCCUUUGUGACA | 12745 | CTTGTCGTCGCCTTTGTGACA |
| 7081 | CUUGUGACAUCUUCACGCGGG | 12746 | CTTGTGACATCTTCACGCGGG |
| 7082 | CUUGUUGGGUCCCUCGCAGAG | 12747 | CTTGTTGGGTCCCTCGCAGAG |
| 7083 | CUUUAGCCUGACGGUGGAUGU | 12748 | CTTTAGCCTGACGGTGGATGT |
| 7084 | CUUUCAUCUUCCAUGUUUGAG | 12749 | CTTTCATCTTCCATGTTTGAG |
| 7085 | CUUUCCCUGGCCUGGGAUCCC | 12750 | CTTTCCCTGGCCTGGGATCCC |
| 7086 | CUUUCCUGGCUUAAGGAAGGU | 12751 | CTTTCCTGGCTTAAGGAAGGT |
| 7087 | CUUUCGCAUCUGUCGCCCACG | 12752 | CTTTCGCATCTGTCGCCCACG |
| 7088 | CUUUCGCAUCUGUCGCCCACU | 12753 | CTTTCGCATCTGTCGCCCACT |
| 7089 | CUUUCUUGAUCUUGGCGGGAG | 12754 | CTTTCTTGATCTTGGCGGGAG |
| 7090 | CUUUGAAGUCCCGGUCAACCU | 12755 | CTTTGAAGTCCCGGTCAACCT |
| 7091 | CUUUGAAUAAAACAAGGCCGG | 12756 | CTTTGAATAAAACAAGGCCGG |
| 7092 | CUUUGAUGGGUUCAUCUGACC | 12757 | CTTTGATGGGTTCATCTGACC |
| 7093 | CUUUGGUCUUCUCUGUCUUUG | 12758 | CTTTGGTCTTCTCTGTCTTTG |
| 7094 | CUUUGUACAUAAAGAAGAGGC | 12759 | CTTTGTACATAAAGAAGAGGC |
| 7095 | CUUUGUCCAGGGUGAUGCAUU | 12760 | CTTTGTCCAGGGTGATGCATT |
| 7096 | CUUUGUCCUCAAAGACGGCCA | 12761 | CTTTGTCCTCAAAGACGGCCA |
| 7097 | CUUUGUGACAGGAACCGCGGA | 12762 | CTTTGTGACAGGAACCGCGGA |
| 7098 | CUUUGUUGCUCAGGCCUGGUC | 12763 | CTTTGTTGCTCAGGCCTGGTC |
| 7099 | CUUUUCAUCCUCCAAGAUGGU | 12764 | CTTTTCATCCTCCAAGATGGT |
| 7100 | CUUUUUGGUUUUUGUAGAGAU | 12765 | CTTTTTGGTTTTTGTAGAGAT |
| 7101 | GAAAACAGUGCAAAAUAAUAA | 12766 | GAAAACAGTGCAAAATAATAA |
| 7102 | GAAAAUGGCUUCGUUGAUGAU | 12767 | GAAAATGGCTTCGTTGATGAT |
| 7103 | GAAACCAAAAUCCCAACCCAA | 12768 | GAAACCAAAATCCCAACCCAA |
| 7104 | GAAACCAGAAAGGCUUAUCAC | 12769 | GAAACCAGAAAGGCTTATCAC |
| 7105 | GAAACCCUGGCUUCCCGCGAU | 12770 | GAAACCCTGGCTTCCCGCGAT |
| 7106 | GAAACCGGGGACUUCGCCAGG | 12771 | GAAACCGGGGACTTCGCCAGG |
| 7107 | GAAACUCAAAACUUCCUGGAG | 12772 | GAAACTCAAAACTTCCTGGAG |
| 7108 | GAAACUCGUCCUGGGAGCACG | 12773 | GAAACTCGTCCTGGGAGCACG |
| 7109 | GAAAGAACCUGAAGUCCCGUC | 12774 | GAAAGAACCTGAAGTCCCGTC |
| 7110 | GAAAGACACUGGGUGGGUCAU | 12775 | GAAAGACACTGGGTGGGTCAT |
| 7111 | GAAAGCAGUGGCAUAAUCAUA | 12776 | GAAAGCAGTGGCATAATCATA |
| 7112 | GAAAGCCACUUGCUUCUCUGG | 12777 | GAAAGCCACTTGCTTCTCTGG |
| 7113 | GAAAGGAAGAAACCAAAAUCC | 12778 | GAAAGGAAGAAACCAAAATCC |
| 7114 | GAAAGGCUUAUCACGGUUCCC | 12779 | GAAAGGCTTATCACGGTTCCC |
| 7115 | GAAAGUGCAAGGAGACCACGG | 12780 | GAAAGTGCAAGGAGACCACGG |
| 7116 | GAAAUAGGGUCUUCAUCUGUU | 12781 | GAAATAGGGTCTTCATCTGTT |
| 7117 | GAAAUGGAGGUGUCAUCCUGG | 12782 | GAAATGGAGGTGTCATCCTGG |
| 7118 | GAAAUUUAAUUGAUCACAGUA | 12783 | GAAATTTAATTGATCACAGTA |
| 7119 | GAACAAAUACAGCAACCAGGG | 12784 | GAACAAATACAGCAACCAGGG |
| 7120 | GAACACGUAAAGACCCCUACA | 12785 | GAACACGTAAAGACCCCTACA |
| 7121 | GAACAGCCGCCGUUGUUGUCC | 12786 | GAACAGCCGCCGTTGTTGTCC |
| 7122 | GAACAGGAUCCACCACGAUGG | 12787 | GAACAGGATCCACCACGATGG |
| 7123 | GAACAGGUCGGGUGGUUGUGU | 12788 | GAACAGGTCGGGTGGTTGTGT |
| 7124 | GAACCAUAUCCUCUGGGGACA | 12789 | GAACCATATCCTCTGGGGACA |
| 7125 | GAACCGAGGCCUUGAGGGUGG | 12790 | GAACCGAGGCCTTGAGGGTGG |
| 7126 | GAACCGCGGAGGAACGCCAGG | 12791 | GAACCGCGGAGGAACGCCAGG |
| 7127 | GAACCUGAAGUCCCGUCAAAC | 12792 | GAACCTGAAGTCCCGTCAAAC |
| 7128 | GAACCUGUGAGGCGGUUGGCA | 12793 | GAACCTGTGAGGCGGTTGGCA |
| 7129 | GAACUCAAGUGAUCCAGCUCA | 12794 | GAACTCAAGTGATCCAGCTCA |
| 7130 | GAACUCGAAGGCCGAGCAGGG | 12795 | GAACTCGAAGGCCGAGCAGGG |
| 7131 | GAACUCGUUUCUUUCGCAUCU | 12796 | GAACTCGTTTCTTTCGCATCT |
| 7132 | GAACUGAGAAAGUGCAAGGAG | 12797 | GAACTGAGAAAGTGCAAGGAG |
| 7133 | GAACUGAGGAAUGCAGCGGUU | 12798 | GAACTGAGGAATGCAGCGGTT |
| 7134 | GAACUGCCGAGAGAUGCACUU | 12799 | GAACTGCCGAGAGATGCACTT |
| 7135 | GAACUUGAUCGGGGCUGGUGG | 12800 | GAACTTGATCGGGGCTGGTGG |
| 7136 | GAACUUGUUGGGUCCCUCGCA | 12801 | GAACTTGTTGGGTCCCTCGCA |
| 7137 | GAAGAAACCAAAAUCCCAACC | 12802 | GAAGAAACCAAAATCCCAACC |
| 7138 | GAAGAAGAGGUAGGCGAUGGA | 12803 | GAAGAAGAGGTAGGCGATGGA |
| 7139 | GAAGACCCCCAGGCAAAGGAA | 12804 | GAAGACCCCCAGGCAAAGGAA |
| 7140 | GAAGACGAGGAGCACGAUGGG | 12805 | GAAGACGAGGAGCACGATGGG |
| 7141 | GAAGACGAGGAGCACUGUAGG | 12806 | GAAGACGAGGAGCACTGTAGG |
| 7142 | GAAGAGAACCAUAUCCUCUGG | 12807 | GAAGAGAACCATATCCTCTGG |
| 7143 | GAAGAGACGCCGUGGGCUCUG | 12808 | GAAGAGACGCCGTGGGCTCTG |
| 7144 | GAAGAGGCAUUUCAGAGGCAA | 12809 | GAAGAGGCATTTCAGAGGCAA |
| 7145 | GAAGAGGUAAACACGUUGACU | 12810 | GAAGAGGTAAACACGTTGACT |
| 7146 | GAAGAGGUAGGCGAUGGAGCC | 12811 | GAAGAGGTAGGCGATGGAGCC |
| 7147 | GAAGAUCUCGGCUGCAGACCA | 12812 | GAAGATCTCGGCTGCAGACCA |
| 7148 | GAAGCACUAGGUGGGCGGACA | 12813 | GAAGCACTAGGTGGGCGGACA |
| 7149 | GAAGCCACUCAUACAUACAAC | 12814 | GAAGCCACTCATACATACAAC |
| 7150 | GAAGCCAUGAACAGGAUCCAC | 12815 | GAAGCCATGAACAGGATCCAC |
| 7151 | GAAGCCGUCGGGGCACAGGCA | 12816 | GAAGCCGTCGGGGCACAGGCA |
| 7152 | GAAGCCUGUUCUGCCUCCCAG | 12817 | GAAGCCTGTTCTGCCTCCCAG |
| 7153 | GAAGCCUUCCUCACACUGGCA | 12818 | GAAGCCTTCCTCACACTGGCA |
| 7154 | GAAGCUGGCGGGACCACAGGU | 12819 | GAAGCTGGCGGGACCACAGGT |
| 7155 | GAAGGAAGACCCCCAGGCAAA | 12820 | GAAGGAAGACCCCCAGGCAAA |
| 7156 | GAAGGCCGAGCAGGGGCUACU | 12821 | GAAGGCCGAGCAGGGGCTACT |
| 7157 | GAAGGGGUGGGCCAGCCUCUU | 12822 | GAAGGGGTGGGCCAGCCTCTT |
| 7158 | GAAGGGUUCUGGGCAGGGACG | 12823 | GAAGGGTTCTGGGCAGGGACG |
| 7159 | GAAGGUACAAACCCACCUCGA | 12824 | GAAGGTACAAACCCACCTCGA |
| 7160 | GAAGUCCCCGGAUUUGCAGGU | 12825 | GAAGTCCCCGGATTTGCAGGT |
| 7161 | GAAGUCCCGGUCAACCUGCCC | 12826 | GAAGTCCCGGTCAACCTGCCC |
| 7162 | GAAGUCCCGUCAAACGAUCCA | 12827 | GAAGTCCCGTCAAACGATCCA |
| 7163 | GAAGUGAAUGGCUUGGAGGCA | 12828 | GAAGTGAATGGCTTGGAGGCA |
| 7164 | GAAGUGGAAGCACUAGGUGGG | 12829 | GAAGTGGAAGCACTAGGTGGG |
| 7165 | GAAGUGGCAUCAUUUGGUGAA | 12830 | GAAGTGGCATCATTTGGTGAA |
| 7166 | GAAGUGGGUAGGGGUCGGGAG | 12831 | GAAGTGGGTAGGGGTCGGGAG |
| 7167 | GAAGUUUGGAGUCAACCCAGU | 12832 | GAAGTTTGGAGTCAACCCAGT |
| 7168 | GAAUAAAACAAGGCCGGCGAG | 12833 | GAATAAAACAAGGCCGGCGAG |
| 7169 | GAAUAAAUAUAUAAAACAAAG | 12834 | GAATAAATATATAAAACAAAG |
| 7170 | GAAUAACGUUUUCCUCUUCAC | 12835 | GAATAACGTTTTCCTCTTCAC |
| 7171 | GAAUAGUCACUGAACAAAUAC | 12836 | GAATAGTCACTGAACAAATAC |
| 7172 | GAAUCCCUUUCCUGGCUUAAG | 12837 | GAATCCCTTTCCTGGCTTAAG |
| 7173 | GAAUGAGUUUGGUCUCGGUGC | 12838 | GAATGAGTTTGGTCTCGGTGC |
| 7174 | GAAUGCAGCGGUUGACACGGC | 12839 | GAATGCAGCGGTTGACACGGC |
| 7175 | GAAUGGAAGUGGGUAGGGGUC | 12840 | GAATGGAAGTGGGTAGGGGTC |
| 7176 | GAAUGGCUUGGAGGCAUUUGC | 12841 | GAATGGCTTGGAGGCATTTGC |
| 7177 | GAAUGUUUUCAGUCACCAGCG | 12842 | GAATGTTTTCAGTCACCAGCG |
| 7178 | GAAUUCGUCAGGGCGACAGGU | 12843 | GAATTCGTCAGGGCGACAGGT |
| 7179 | GACAAGAUUGGGGAAGUGAAU | 12844 | GACAAGATTGGGGAAGTGAAT |
| 7180 | GACAAGCACGUCUCCUGGGAC | 12845 | GACAAGCACGTCTCCTGGGAC |
| 7181 | GACAAGGUGGGGACGGAGGUG | 12846 | GACAAGGTGGGGACGGAGGTG |
| 7182 | GACAAUGGACAGAGCCCUCAC | 12847 | GACAATGGACAGAGCCCTCAC |
| 7183 | GACAAUGGCAGUUCUCGAUAU | 12848 | GACAATGGCAGTTCTCGATAT |
| 7184 | GACAAUUGCCAAUCCCUUGUG | 12849 | GACAATTGCCAATCCCTTGTG |
| 7185 | GACACAGUGUUUUGUCCCUGG | 12850 | GACACAGTGTTTTGTCCCTGG |
| 7186 | GACACCCAUCUCCCAGAAGCC | 12851 | GACACCCATCTCCCAGAAGCC |
| 7187 | GACACGGCCCCCACAGCUGAA | 12852 | GACACGGCCCCCACAGCTGAA |
| 7188 | GACACGGGCUUUCCCUGGCCU | 12853 | GACACGGGCTTTCCCTGGCCT |
| 7189 | GACACUCAUCGAUAUCGCACU | 12854 | GACACTCATCGATATCGCACT |
| 7190 | GACACUCAUCGAUAUCUUCGC | 12855 | GACACTCATCGATATCTTCGC |
| 7191 | GACACUGGGUGGGUCAUGAUU | 12856 | GACACTGGGTGGGTCATGATT |
| 7192 | GACACUUGAACUUGUUGGGUC | 12857 | GACACTTGAACTTGTTGGGTC |
| 7193 | GACAGAAAACAGUGCAAAAUA | 12858 | GACAGAAAACAGTGCAAAATA |
| 7194 | GACAGACAAGCACGUCUCCUG | 12859 | GACAGACAAGCACGTCTCCTG |
| 7195 | GACAGAGCCCUCACGCUACUG | 12860 | GACAGAGCCCTCACGCTACTG |
| 7196 | GACAGAGUCGGUCCAGUAGAU | 12861 | GACAGAGTCGGTCCAGTAGAT |
| 7197 | GACAGCCACACCUGGGUGCAG | 12862 | GACAGCCACACCTGGGTGCAG |
| 7198 | GACAGCCUCAAAUGCCUGAGC | 12863 | GACAGCCTCAAATGCCTGAGC |
| 7199 | GACAGCCUUGCUCGUCUGAGC | 12864 | GACAGCCTTGCTCGTCTGAGC |
| 7200 | GACAGGAACCGCGGAGGAACG | 12865 | GACAGGAACCGCGGAGGAACG |
| 7201 | GACAGGGCAAAGGCUAACCUG | 12866 | GACAGGGCAAAGGCTAACCTG |
| 7202 | GACAGGUCAGACCAGUAGAUU | 12867 | GACAGGTCAGACCAGTAGATT |
| 7203 | GACAGGUGGCCACAGCGCAGU | 12868 | GACAGGTGGCCACAGCGCAGT |
| 7204 | GACAGGUGGCCACAGGACAGC | 12869 | GACAGGTGGCCACAGGACAGC |
| 7205 | GACAGUAGGUUUUCAGCCAAC | 12870 | GACAGTAGGTTTTCAGCCAAC |
| 7206 | GACAGUGCCCAGGACAGAGUC | 12871 | GACAGTGCCCAGGACAGAGTC |
| 7207 | GACAUAAAGACGACAAUGGCA | 12872 | GACATAAAGACGACAATGGCA |
| 7208 | GACAUCAGUGCAACAGUAACA | 12873 | GACATCAGTGCAACAGTAACA |
| 7209 | GACAUCAUUCUCCCAAAAAGC | 12874 | GACATCATTCTCCCAAAAAGC |
| 7210 | GACAUCCCCUCCCCUUUCAUC | 12875 | GACATCCCCTCCCCTTTCATC |
| 7211 | GACAUCGAUGCUUGAGAUGGA | 12876 | GACATCGATGCTTGAGATGGA |
| 7212 | GACAUCGGAACCUGUGAGGCG | 12877 | GACATCGGAACCTGTGAGGCG |
| 7213 | GACAUCUUCACGCGGGAGUCU | 12878 | GACATCTTCACGCGGGAGTCT |
| 7214 | GACAUUCAGGCUACAAAAACC | 12879 | GACATTCAGGCTACAAAAACC |
| 7215 | GACAUUGUCACUAUCUCCACC | 12880 | GACATTGTCACTATCTCCACC |
| 7216 | GACCAAGGAGGGGGGGGGAGA | 12881 | GACCAAGGAGGGGGGGGAGA |
| 7217 | GACCACAGGUGAGCACCGGGC | 12882 | GACCACAGGTGAGCACCGGGC |
| 7218 | GACCACGGGAAUGGAAGUGGG | 12883 | GACCACGGGAATGGAAGTGGG |
| 7219 | GACCACGUUCCUCAGGUUGGG | 12884 | GACCACGTTCCTCAGGTTGGG |
| 7220 | GACCAGAGACCCAGCUCCGUG | 12885 | GACCAGAGACCCAGCTCCGTG |
| 7221 | GACCAGUAGAUUCUAUUGCUG | 12886 | GACCAGTAGATTCTATTGCTG |
| 7222 | GACCAGUCCCGGCAGUCUCUA | 12887 | GACCAGTCCCGGCAGTCTCTA |
| 7223 | GACCAGUGACUCAGGCUCUGC | 12888 | GACCAGTGACTCAGGCTCTGC |
| 7224 | GACCAUCGAGGGGUAGCUGUA | 12889 | GACCATCGAGGGGTAGCTGTA |
| 7225 | GACCAUCUGUCUCGAGGGGUA | 12890 | GACCATCTGTCTCGAGGGGTA |
| 7226 | GACCCACUUGUAGGAGAUGCA | 12891 | GACCCACTTGTAGGAGATGCA |
| 7227 | GACCCAGCUCCGUGGCGAUGG | 12892 | GACCCAGCTCCGTGGCGATGG |
| 7228 | GACCCCCAGGCAAAGGAAGAC | 12893 | GACCCCCAGGCAAAGGAAGAC |
| 7229 | GACCCCUACAGCGCUGCGGCC | 12894 | GACCCCTACAGCGCTGCGGCC |
| 7230 | GACCCGGAUCACGACCUGCUG | 12895 | GACCCGGATCACGACCTGCTG |
| 7231 | GACCCGGGACUUGUCGUCGCC | 12896 | GACCCGGGACTTGTCGTCGCC |
| 7232 | GACCUCAUCCUCUGUGGUCUU | 12897 | GACCTCATCCTCTGTGGTCTT |
| 7233 | GACCUCAUGAUCCACCCGCCU | 12898 | GACCTCATGATCCACCCGCCT |
| 7234 | GACCUCGUGCCGGUUGGUGAA | 12899 | GACCTCGTGCCGGTTGGTGAA |
| 7235 | GACCUGCUGUGUCCUAGCUGG | 12900 | GACCTGCTGTGTCCTAGCTGG |
| 7236 | GACCUUUAGCCUGACGGUGGA | 12901 | GACCTTTAGCCTGACGGTGGA |
| 7237 | GACGAACUGCCGAGAGAUGCA | 12902 | GACGAACTGCCGAGAGATGCA |
| 7238 | GACGACAAUGGCAGUUCUCGA | 12903 | GACGACAATGGCAGTTCTCGA |
| 7239 | GACGAGGAGCACGAUGGGGAG | 12904 | GACGAGGAGCACGATGGGGAG |
| 7240 | GACGAGGAGCACUGUAGGGAG | 12905 | GACGAGGAGCACTGTAGGGAG |
| 7241 | GACGCAUUUACGUGCUCCGAA | 12906 | GACGCATTTACGTGCTCCGAA |
| 7242 | GACGCCAUGAAUCCCUUUCCU | 12907 | GACGCCATGAATCCCTTTCCT |
| 7243 | GACGCCGUGGGCUCUGUCAAG | 12908 | GACGCCGTGGGCTCTGTCAAG |
| 7244 | GACGGAGGCUGUCACUGUCUC | 12909 | GACGGAGGCTGTCACTGTCTC |
| 7245 | GACGGAGGUGUCGCCACAGAG | 12910 | GACGGAGGTGTCGCCACAGAG |
| 7246 | GACGGAGUCUCGCUCUGUCGC | 12911 | GACGGAGTCTCGCTCTGTCGC |
| 7247 | GACGGCCAAGGAGAAGGGGUG | 12912 | GACGGCCAAGGAGAAGGGGTG |
| 7248 | GACGGGACUCCAGGCAGAUGU | 12913 | GACGGGACTCCAGGCAGATGT |
| 7249 | GACGGGGUUGUCAAAGUUGAU | 12914 | GACGGGGTTGTCAAAGTTGAT |
| 7250 | GACGGGGUUUCACGUGUUAGC | 12915 | GACGGGGTTTCACGTGTTAGC |
| 7251 | GACGGUCCAGCGCAAUUUCCA | 12916 | GACGGTCCAGCGCAATTTCCA |
| 7252 | GACGGUGGAUGUCUCCUGGGU | 12917 | GACGGTGGATGTCTCCTGGGT |
| 7253 | GACGGUGUCAUAGGAAGAGAC | 12918 | GACGGTGTCATAGGAAGAGAC |
| 7254 | GACGUGGGAACAGCCGCCGUU | 12919 | GACGTGGGAACAGCCGCCGTT |
| 7255 | GACUACAGGUGCCCGCCACCA | 12920 | GACTACAGGTGCCCGCCACCA |
| 7256 | GACUCAGGCUCUGCCUCUGGG | 12921 | GACTCAGGCTCTGCCTCTGGG |
| 7257 | GACUCAUCAGAGCCAUCCUGG | 12922 | GACTCATCAGAGCCATCCTGG |
| 7258 | GACUCCAGGCAGAUGUUCACG | 12923 | GACTCCAGGCAGATGTTCACG |
| 7259 | GACUCCUGAGUUUAGGGGUUU | 12924 | GACTCCTGAGTTTAGGGGTTT |
| 7260 | GACUCGUGUAUGUCGCGGAGG | 12925 | GACTCGTGTATGTCGCGGAGG |
| 7261 | GACUGACCAUCGAGGGGUAGC | 12926 | GACTGACCATCGAGGGGTAGC |
| 7262 | GACUGACCAUCUGUCUCGAGG | 12927 | GACTGACCATCTGTCTCGAGG |
| 7263 | GACUGCAGUGGCGCAAUCUCG | 12928 | GACTGCAGTGGCGCAATCTCG |
| 7264 | GACUGCGGACUGCAGUGGCGC | 12929 | GACTGCGGACTGCAGTGGCGC |
| 7265 | GACUGGAGGAUUUAUUUAUUU | 12930 | GACTGGAGGATTTATTTATTT |
| 7266 | GACUUCGCCAGGAGGGCCCAG | 12931 | GACTTCGCCAGGAGGGCCCAG |
| 7267 | GACUUGUCGUCGCCUUUGUGA | 12932 | GACTTGTCGTCGCCTTTGTGA |
| 7268 | GACUUUGUCCAGGGUGAUGCA | 12933 | GACTTTGTCCAGGGTGATGCA |
| 7269 | GAGAAACUCAAAACUUCCUGG | 12934 | GAGAAACTCAAAACTTCCTGG |
| 7270 | GAGAAAGUGCAAGGAGACCAC | 12935 | GAGAAAGTGCAAGGAGACCAC |
| 7271 | GAGAAAUGGAGGUGUCAUCCU | 12936 | GAGAAATGGAGGTGTCATCCT |
| 7272 | GAGAACCAUAUCCUCUGGGGA | 12937 | GAGAACCATATCCTCTGGGGA |
| 7273 | GAGAAGGGGUGGGCCAGCCUC | 12938 | GAGAAGGGGTGGGCCAGCCTC |
| 7274 | GAGAAUAGUCACUGAACAAAU | 12939 | GAGAATAGTCACTGAACAAAT |
| 7275 | GAGACAGUGCCCAGGACAGAG | 12940 | GAGACAGTGCCCAGGACAGAG |
| 7276 | GAGACAUUGUCACUAUCUCCA | 12941 | GAGACATTGTCACTATCTCCA |
| 7277 | GAGACCACGGGAAUGGAAGUG | 12942 | GAGACCACGGGAATGGAAGTG |
| 7278 | GAGACCAGAGACCCAGCUCCG | 12943 | GAGACCAGAGACCCAGCTCCG |
| 7279 | GAGACCCAGCUCCGUGGCGAU | 12944 | GAGACCCAGCTCCGTGGCGAT |
| 7280 | GAGACGCCGUGGGCUCUGUCA | 12945 | GAGACGCCGTGGGCTCTGTCA |
| 7281 | GAGACGGAGUCUCGCUCUGUC | 12946 | GAGACGGAGTCTCGCTCTGTC |
| 7282 | GAGACGGGGUUUCACGUGUUA | 12947 | GAGACGGGGTTTCACGTGTTA |
| 7283 | GAGAGAAACUCAAAACUUCCU | 12948 | GAGAGAAACTCAAAACTTCCT |
| 7284 | GAGAGAAAUGGAGGUGUCAUC | 12949 | GAGAGAAATGGAGGTGTCATC |
| 7285 | GAGAGAUGCACUUCCCAUCGU | 12950 | GAGAGATGCACTTCCCATCGT |
| 7286 | GAGAGGCACGCUCUGUCACAC | 12951 | GAGAGGCACGCTCTGTCACAC |
| 7287 | GAGAUCUAGGGUGAUGCCAUU | 12952 | GAGATCTAGGGTGATGCCATT |
| 7288 | GAGAUGCACUUCCCAUCGUGG | 12953 | GAGATGCACTTCCCATCGTGG |
| 7289 | GAGAUGCAUUUCCCGUCUUGG | 12954 | GAGATGCATTTCCCGTCTTGG |
| 7290 | GAGAUGGAGUGAAGUUUGGAG | 12955 | GAGATGGAGTGAAGTTTGGAG |
| 7291 | GAGAUGGGAUCUCGCUUUGUU | 12956 | GAGATGGGATCTCGCTTTGTT |
| 7292 | GAGAUGGGGUCUUACUAUGUU | 12957 | GAGATGGGGTCTTACTATGTT |
| 7293 | GAGAUUCUGAGGCAACGUUUC | 12958 | GAGATTCTGAGGCAACGTTTC |
| 7294 | GAGAUUGGUGGAUGAGGCAGG | 12959 | GAGATTGGTGGATGAGGCAGG |
| 7295 | GAGCAAGGCGACGGUCCAGCG | 12960 | GAGCAAGGCGACGGTCCAGCG |
| 7296 | GAGCACAGCGGAAACCGGGGA | 12961 | GAGCACAGCGGAAACCGGGGA |
| 7297 | GAGCACCGGGCAGGAGGCCUC | 12962 | GAGCACCGGGCAGGAGGCCTC |
| 7298 | GAGCACGAUGGGGAGGACAAU | 12963 | GAGCACGATGGGGAGGACAAT |
| 7299 | GAGCACGUCUCCUGGGACUCA | 12964 | GAGCACGTCTCCTGGGACTCA |
| 7300 | GAGCACGUCUUGGGGGAGCAC | 12965 | GAGCACGTCTTGGGGGAGCAC |
| 7301 | GAGCACGUCUUGGGGGGACAG | 12966 | GAGCACGTCTTGGGGGGACAG |
| 7302 | GAGCACUGGAAUUCGUCAGGG | 12967 | GAGCACTGGAATTCGTCAGGG |
| 7303 | GAGCACUGUAGGGAGAGGCAC | 12968 | GAGCACTGTAGGGAGAGGCAC |
| 7304 | GAGCAGGGGCUACUGUCCCCU | 12969 | GAGCAGGGGCTACTGTCCCCT |
| 7305 | GAGCCACUGCGCCCGGCCUCA | 12970 | GAGCCACTGCGCCCGGCCTCA |
| 7306 | GAGCCAUCCUGGCACUCAGCG | 12971 | GAGCCATCCTGGCACTCAGCG |
| 7307 | GAGCCAUCUUCGCAGUCGGGG | 12972 | GAGCCATCTTCGCAGTCGGGG |
| 7308 | GAGCCCACAGCCUUGCAGGCC | 12973 | GAGCCCACAGCCTTGCAGGCC |
| 7309 | GAGCCCAGGGGUGGCCCCAGG | 12974 | GAGCCCAGGGGTGGCCCCAGG |
| 7310 | GAGCCCUCACGCUACUGGGCU | 12975 | GAGCCCTCACGCTACTGGGCT |
| 7311 | GAGCCGUCCAAGCAGUCCCGG | 12976 | GAGCCGTCCAAGCAGTCCCGG |
| 7312 | GAGCCGUUCUCCCUGAAUAAC | 12977 | GAGCCGTTCTCCCTGAATAAC |
| 7313 | GAGCCGUUGUCGCAGUCCACU | 12978 | GAGCCGTTGTCGCAGTCCACT |
| 7314 | GAGCGACCACGUUCCUCAGGU | 12979 | GAGCGACCACGTTCCTCAGGT |
| 7315 | GAGCUCAAACCAUCCUCCCAC | 12980 | GAGCTCAAACCATCCTCCCAC |
| 7316 | GAGCUGACCUUUAGCCUGACG | 12981 | GAGCTGACCTTTAGCCTGACG |
| 7317 | GAGCUGGCUGCAGGUGUCGGG | 12982 | GAGCTGGCTGCAGGTGTCGGG |
| 7318 | GAGCUGUUGCACUGGAAGCUG | 12983 | GAGCTGTTGCACTGGAAGCTG |
| 7319 | GAGCUUGGUGAGACAUUGUCA | 12984 | GAGCTTGGTGAGACATTGTCA |
| 7320 | GAGGAAAGGAAGAAACCAAAA | 12985 | GAGGAAAGGAAGAAACCAAAA |
| 7321 | GAGGAACGCCAGGCCUCCCCU | 12986 | GAGGAACGCCAGGCCTCCCCT |
| 7322 | GAGGAAUGCAGCGGUUGACAC | 12987 | GAGGAATGCAGCGGTTGACAC |
| 7323 | GAGGACAAUGGACAGAGCCCU | 12988 | GAGGACAATGGACAGAGCCCT |
| 7324 | GAGGAGAUCUAGGGUGAUGCC | 12989 | GAGGAGATCTAGGGTGATGCC |
| 7325 | GAGGAGCAAGGCGACGGUCCA | 12990 | GAGGAGCAAGGCGACGGTCCA |
| 7326 | GAGGAGCACGAUGGGGAGGAC | 12991 | GAGGAGCACGATGGGGAGGAC |
| 7327 | GAGGAGCACUGUAGGGAGAGG | 12992 | GAGGAGCACTGTAGGGAGAGG |
| 7328 | GAGGAUGGGCACCAGGCUGGU | 12993 | GAGGATGGGCACCAGGCTGGT |
| 7329 | GAGGAUUGUGUCACGGUGGAG | 12994 | GAGGATTGTGTCACGGTGGAG |
| 7330 | GAGGAUUUAUUUAUUUUUUUA | 12995 | GAGGATTTATTTATTTTTTTA |
| 7331 | GAGGCAACGUUUCAACCUCGU | 12996 | GAGGCAACGTTTCAACCTCGT |
| 7332 | GAGGCAAUAACCCCCUACACA | 12997 | GAGGCAATAACCCCCTACACA |
| 7333 | GAGGCACAGAUACUGGCAGCC | 12998 | GAGGCACAGATACTGGCAGCC |
| 7334 | GAGGCACCUGUGUCUGGCCAA | 12999 | GAGGCACCTGTGTCTGGCCAA |
| 7335 | GAGGCACGCUCUGUCACACAG | 13000 | GAGGCACGCTCTGTCACACAG |
| 7336 | GAGGCAGCUCCUCAUGUCCCU | 13001 | GAGGCAGCTCCTCATGTCCCT |
| 7337 | GAGGCAGGGCCAGGGGGUCCC | 13002 | GAGGCAGGGCCAGGGGGTCCC |
| 7338 | GAGGCAUUUCAGAGGCAAUAA | 13003 | GAGGCATTTCAGAGGCAATAA |
| 7339 | GAGGCAUUUGCAUAGAAGUGG | 13004 | GAGGCATTTGCATAGAAGTGG |
| 7340 | GAGGCCAUGGGCUGCUGAUGC | 13005 | GAGGCCATGGGCTGCTGATGC |
| 7341 | GAGGCCUCGUCUGAGCCGUCC | 13006 | GAGGCCTCGTCTGAGCCGTCC |
| 7342 | GAGGCCUUGAGGGUGGCCGGG | 13007 | GAGGCCTTGAGGGTGGCCGGG |
| 7343 | GAGGCGGCCACUGAGGAGAUC | 13008 | GAGGCGGCCACTGAGGAGATC |
| 7344 | GAGGCGGCUCCGGGGACCCGG | 13009 | GAGGCGGCTCCGGGGACCCGG |
| 7345 | GAGGCGGUUGGCACUGAAAAU | 13010 | GAGGCGGTTGGCACTGAAAAT |
| 7346 | GAGGCUGGUGUACUCGCUCCG | 13011 | GAGGCTGGTGTACTCGCTCCG |
| 7347 | GAGGCUGUCACUGUCUCUCCG | 13012 | GAGGCTGTCACTGTCTCTCCG |
| 7348 | GAGGGCCCAGGAUAACGGAGG | 13013 | GAGGGCCCAGGATAACGGAGG |
| 7349 | GAGGGCGACUCGUGUAUGUCG | 13014 | GAGGGCGACTCGTGTATGTCG |
| 7350 | GAGGGCGCCCCCCAGGACAAG | 13015 | GAGGGCGCCCCCCAGGACAAG |
| 7351 | GAGGGGGGGGGAGAUUCUGAG | 13016 | GAGGGGGGGGGAGATTCTGAG |
| 7352 | GAGGGGUAGCUGUAGCCGUCC | 13017 | GAGGGGTAGCTGTAGCCGTCC |
| 7353 | GAGGGUGGCCGGGGGCCCCUC | 13018 | GAGGGTGGCCGGGGGCCCCTC |
| 7354 | GAGGUAAACACGUUGACUCCU | 13019 | GAGGTAAACACGTTGACTCCT |
| 7355 | GAGGUAGGCGAUGGAGCCCAC | 13020 | GAGGTAGGCGATGGAGCCCAC |
| 7356 | GAGGUCUCAGGAAGGGUUCUG | 13021 | GAGGTCTCAGGAAGGGTTCTG |
| 7357 | GAGGUGUCAUCCUGGUCCCCC | 13022 | GAGGTGTCATCCTGGTCCCCC |
| 7358 | GAGGUGUCGCCACAGAGCACA | 13023 | GAGGTGTCGCCACAGAGCACA |
| 7359 | GAGGUGUCGGGAACAGGUCGG | 13024 | GAGGTGTCGGGAACAGGTCGG |
| 7360 | GAGGUUGUGGAAGAGAACCAU | 13025 | GAGGTTGTGGAAGAGAACCAT |
| 7361 | GAGUAGAUGUCCACACCAUUC | 13026 | GAGTAGATGTCCACACCATTC |
| 7362 | GAGUAGCUGGGACUACAGGUG | 13027 | GAGTAGCTGGGACTACAGGTG |
| 7363 | GAGUCAACCCAGUAGAGGCGG | 13028 | GAGTCAACCCAGTAGAGGCGG |
| 7364 | GAGUCACAGACGAACUGCCGA | 13029 | GAGTCACAGACGAACTGCCGA |
| 7365 | GAGUCGGUCCAGUAGAUGUUG | 13030 | GAGTCGGTCCAGTAGATGTTG |
| 7366 | GAGUCUCGCUCUGUCGCCCAG | 13031 | GAGTCTCGCTCTGTCGCCCAG |
| 7367 | GAGUCUGACGGAGGCUGUCAC | 13032 | GAGTCTGACGGAGGCTGTCAC |
| 7368 | GAGUGAAGUUUGGAGUCAACC | 13033 | GAGTGAAGTTTGGAGTCAACC |
| 7369 | GAGUGCAGUGGCUCGAUCAUG | 13034 | GAGTGCAGTGGCTCGATCATG |
| 7370 | GAGUGGAUGCACUCGCCACUU | 13035 | GAGTGGATGCACTCGCCACTT |
| 7371 | GAGUGGGGGUUGAUCUGCGGG | 13036 | GAGTGGGGGTTGATCTGCGGG |
| 7372 | GAGUGUCACAUUAACGCAGCC | 13037 | GAGTGTCACATTAACGCAGCC |
| 7373 | GAGUGUCAGACAGCCUUGCUC | 13038 | GAGTGTCAGACAGCCTTGCTC |
| 7374 | GAGUUCCCCAGUCAGUCCAGU | 13039 | GAGTTCCCCAGTCAGTCCAGT |
| 7375 | GAGUUUAGGGGUUUGGCUUAG | 13040 | GAGTTTAGGGGTTTGGCTTAG |
| 7376 | GAGUUUGGUCUCGGUGCCACC | 13041 | GAGTTTGGTCTCGGTGCCACC |
| 7377 | GAUAACAGUUUCAACCAAUAU | 13042 | GATAACAGTTTCAACCAATAT |
| 7378 | GAUAACGGAGGGCGACUCGUG | 13043 | GATAACGGAGGGCGACTCGTG |
| 7379 | GAUACUGGCAGCCGCCAUUGC | 13044 | GATACTGGCAGCCGCCATTGC |
| 7380 | GAUAGACGGGGUUGUCAAAGU | 13045 | GATAGACGGGGTTGTCAAAGT |
| 7381 | GAUAUCGCACUCUUUGAUGGG | 13046 | GATATCGCACTCTTTGATGGG |
| 7382 | GAUAUCUGUCCAAAAUACUUU | 13047 | GATATCTGTCCAAAATACTTT |
| 7383 | GAUAUCUUCGCAUCUUCGCUG | 13048 | GATATCTTCGCATCTTCGCTG |
| 7384 | GAUAUUCACGAUCACGGCUUU | 13049 | GATATTCACGATCACGGCTTT |
| 7385 | GAUCACAGGCACGUGCCACCG | 13050 | GATCACAGGCACGTGCCACCG |
| 7386 | GAUCACAGUAAGACAUUCAGG | 13051 | GATCACAGTAAGACATTCAGG |
| 7387 | GAUCACGACCUGCUGUGUCCU | 13052 | GATCACGACCTGCTGTGTCCT |
| 7388 | GAUCACGGCUUUGAAGUCCCG | 13053 | GATCACGGCTTTGAAGTCCCG |
| 7389 | GAUCAUGGCUCACUGCAACCU | 13054 | GATCATGGCTCACTGCAACCT |
| 7390 | GAUCAUUCUCUGGGACAGGUC | 13055 | GATCATTCTCTGGGACAGGTC |
| 7391 | GAUCAUUUCCGACGCCAUGAA | 13056 | GATCATTTCCGACGCCATGAA |
| 7392 | GAUCCACCACGAUGGCCCUUG | 13057 | GATCCACCACGATGGCCCTTG |
| 7393 | GAUCCACCCGCCUCGGCCUCC | 13058 | GATCCACCCGCCTCGGCCTCC |
| 7394 | GAUCCAGACUGGAGGAUUUAU | 13059 | GATCCAGACTGGAGGATTTAT |
| 7395 | GAUCCAGCUCAGCCUCCCAAA | 13060 | GATCCAGCTCAGCCTCCCAAA |
| 7396 | GAUCCAGUCCACAGCCAGCCC | 13061 | GATCCAGTCCACAGCCAGCCC |
| 7397 | GAUCCCAUCCCAACACACACG | 13062 | GATCCCATCCCAACACACACG |
| 7398 | GAUCCUCCUGCCUCAGCCUCC | 13063 | GATCCTCCTGCCTCAGCCTCC |
| 7399 | GAUCCUGACACUCAUCGAUAU | 13064 | GATCCTGACACTCATCGATAT |
| 7400 | GAUCGGGGCUGGUGGGGCGAG | 13065 | GATCGGGGCTGGTGGGGCGAG |
| 7401 | GAUCUAGGGUGAUGCCAUUGG | 13066 | GATCTAGGGTGATGCCATTGG |
| 7402 | GAUCUCCUGACCUCAUGAUCC | 13067 | GATCTCCTGACCTCATGATCC |
| 7403 | GAUCUCGCUUUGUUGCUCAGG | 13068 | GATCTCGCTTTGTTGCTCAGG |
| 7404 | GAUCUCGGCUGCAGACCAAGG | 13069 | GATCTCGGCTGCAGACCAAGG |
| 7405 | GAUCUGCGGGGCAGGGAGGCA | 13070 | GATCTGCGGGGCAGGGAGGCA |
| 7406 | GAUCUUAAGGUCAUUGCAGAC | 13071 | GATCTTAAGGTCATTGCAGAC |
| 7407 | GAUCUUGGCGGGAGUUCCCCA | 13072 | GATCTTGGCGGGAGTTCCCCA |
| 7408 | GAUGAAUAAAUAUAUAAAACA | 13073 | GATGAATAAATATATAAAACA |
| 7409 | GAUGACGGUGUCAUAGGAAGA | 13074 | GATGACGGTGTCATAGGAAGA |
| 7410 | GAUGAGGCAGGGCCAGGGGGU | 13075 | GATGAGGCAGGGCCAGGGGGT |
| 7411 | GAUGAGGCUGGUGUACUCGCU | 13076 | GATGAGGCTGGTGTACTCGCT |
| 7412 | GAUGAUAUCUGUCCAAAAUAC | 13077 | GATGATATCTGTCCAAAATAC |
| 7413 | GAUGCACCAUGCAUGGUUUUU | 13078 | GATGCACCATGCATGGTTTTT |
| 7414 | GAUGCACUCGCCACUUAGGCA | 13079 | GATGCACTCGCCACTTAGGCA |
| 7415 | GAUGCACUUCCCAUCGUGGCA | 13080 | GATGCACTTCCCATCGTGGCA |
| 7416 | GAUGCAGGUGGAGCUGUUGCA | 13081 | GATGCAGGTGGAGCTGTTGCA |
| 7417 | GAUGCAGUUUCCAUCAGAGCA | 13082 | GATGCAGTTTCCATCAGAGCA |
| 7418 | GAUGCAUUCGCCGCUGUGACA | 13083 | GATGCATTCGCCGCTGTGACA |
| 7419 | GAUGCAUUUCCCGUCUUGGCA | 13084 | GATGCATTTCCCGTCTTGGCA |
| 7420 | GAUGCCAUUGGGCCACUGAAU | 13085 | GATGCCATTGGGCCACTGAAT |
| 7421 | GAUGCUGUUGAUGUUCUUAAG | 13086 | GATGCTGTTGATGTTCTTAAG |
| 7422 | GAUGCUUGAGAUGGAGUGAAG | 13087 | GATGCTTGAGATGGAGTGAAG |
| 7423 | GAUGGAGCCCACAGCCUUGCA | 13088 | GATGGAGCCCACAGCCTTGCA |
| 7424 | GAUGGAGUGAAGUUUGGAGUC | 13089 | GATGGAGTGAAGTTTGGAGTC |
| 7425 | GAUGGCCCUUGGCUUGGAGCC | 13090 | GATGGCCCTTGGCTTGGAGCC |
| 7426 | GAUGGGAUCUCGCUUUGUUGC | 13091 | GATGGGATCTCGCTTTGTTGC |
| 7427 | GAUGGGCACCAGGCUGGUGUC | 13092 | GATGGGCACCAGGCTGGTGTC |
| 7428 | GAUGGGGAGGACAAUGGACAG | 13093 | GATGGGGAGGACAATGGACAG |
| 7429 | GAUGGGGUCUUACUAUGUUGC | 13094 | GATGGGGTCTTACTATGTTGC |
| 7430 | GAUGGGUUCAUCUGACCAGUC | 13095 | GATGGGTTCATCTGACCAGTC |
| 7431 | GAUGGUCUCGAUCUCCUGACC | 13096 | GATGGTCTCGATCTCCTGACC |
| 7432 | GAUGGUCUUCCGGUUGCCCCC | 13097 | GATGGTCTTCCGGTTGCCCCC |
| 7433 | GAUGUCCACACCAUUCAGGCC | 13098 | GATGTCCACACCATTCAGGCC |
| 7434 | GAUGUCUCCUGGGUGGCCACU | 13099 | GATGTCTCCTGGGTGGCCACT |
| 7435 | GAUGUCUCUGCUGAUGACGGU | 13100 | GATGTCTCTGCTGATGACGGT |
| 7436 | GAUGUUCACGCCACGUCAUCC | 13101 | GATGTTCACGCCACGTCATCC |
| 7437 | GAUGUUCUUAAGCCGCCAGUU | 13102 | GATGTTCTTAAGCCGCCAGTT |
| 7438 | GAUGUUGCUGUGGAUCCAGUC | 13103 | GATGTTGCTGTGGATCCAGTC |
| 7439 | GAUUACAGGCGUGAGCCACUG | 13104 | GATTACAGGCGTGAGCCACTG |
| 7440 | GAUUACAGGCGUGAGGCACCU | 13105 | GATTACAGGCGTGAGGCACCT |
| 7441 | GAUUCAGCCAGAUCAUUUCCG | 13106 | GATTCAGCCAGATCATTTCCG |
| 7442 | GAUUCGGGGGUGACAGGGCAA | 13107 | GATTCGGGGGTGACAGGGCAA |
| 7443 | GAUUCUAUUGCUGGCCACCUC | 13108 | GATTCTATTGCTGGCCACCTC |
| 7444 | GAUUCUGAGGCAACGUUUCAA | 13109 | GATTCTGAGGCAACGTTTCAA |
| 7445 | GAUUGGGGAAGUGAAUGGCUU | 13110 | GATTGGGGAAGTGAATGGCTT |
| 7446 | GAUUGGUGGAUGAGGCAGGGC | 13111 | GATTGGTGGATGAGGCAGGGC |
| 7447 | GAUUGUGUCACGGUGGAGAGA | 13112 | GATTGTGTCACGGTGGAGAGA |
| 7448 | GAUUUAUUUAUUUUUUUAUAG | 13113 | GATTTATTTATTTTTTTATAG |
| 7449 | GAUUUCAGAAAGAACCUGAAG | 13114 | GATTTCAGAAAGAACCTGAAG |
| 7450 | GAUUUGCAGGUGACAGACAAG | 13115 | GATTTGCAGGTGACAGACAAG |
| 7451 | GAUUUGUCCUUGCAGUCGGGG | 13116 | GATTTGTCCTTGCAGTCGGGG |
| 7452 | GAUUUUAUAUAUAUGUGUGUA | 13117 | GATTTTATATATATGTGTGTA |
| 7453 | GAUUUUUAACUUUUUGGUUUU | 13118 | GATTTTTAACTTTTTGGTTTT |
| 7454 | GCAAAAAUAAAUAGAUUUUAU | 13119 | GCAAAAATAAATAGATTTTAT |
| 7455 | GCAAAAUAAUAACACAAAUGC | 13120 | GCAAAATAATAACACAAATGC |
| 7456 | GCAAACAUAACAAAAAAUCCC | 13121 | GCAAACATAACAAAAAATCCC |
| 7457 | GCAAAGGAAGACGAGGAGCAC | 13122 | GCAAAGGAAGACGAGGAGCAC |
| 7458 | GCAAAGGCUAACCUGGCUGUC | 13123 | GCAAAGGCTAACCTGGCTGTC |
| 7459 | GCAAAUAAUCUUUGUACAUAA | 13124 | GCAAATAATCTTTGTACATAA |
| 7460 | GCAAAUGUGGACCUCAUCCUC | 13125 | GCAAATGTGGACCTCATCCTC |
| 7461 | GCAACAGAGACAGUGCCCAGG | 13126 | GCAACAGAGACAGTGCCCAGG |
| 7462 | GCAACAGUAACACGGCGAUUU | 13127 | GCAACAGTAACACGGCGATTT |
| 7463 | GCAACCAGGGUUUGCAAAAAU | 13128 | GCAACCAGGGTTTGCAAAAAT |
| 7464 | GCAACCUCCACCUCCUGGGCU | 13129 | GCAACCTCCACCTCCTGGGCT |
| 7465 | GCAACGUCGCCCAGAGCUUGG | 13130 | GCAACGTCGCCCAGAGCTTGG |
| 7466 | GCAACGUUUCAACCUCGUAAG | 13131 | GCAACGTTTCAACCTCGTAAG |
| 7467 | GCAAGCUCCGCUUCCCGGGUU | 13132 | GCAAGCTCCGCTTCCCGGGTT |
| 7468 | GCAAGGAGACCACGGGAAUGG | 13133 | GCAAGGAGACCACGGGAATGG |
| 7469 | GCAAGGCGACGGUCCAGCGCA | 13134 | GCAAGGCGACGGTCCAGCGCA |
| 7470 | GCAAGGCUUGCAUAGAAGAGG | 13135 | GCAAGGCTTGCATAGAAGAGG |
| 7471 | GCAAUAACCCCCUACACAGGG | 13136 | GCAATAACCCCCTACACAGGG |
| 7472 | GCAAUCUCGGCUCACUGCAAG | 13137 | GCAATCTCGGCTCACTGCAAG |
| 7473 | GCAAUGCUUUGGUCUUCUCUG | 13138 | GCAATGCTTTGGTCTTCTCTG |
| 7474 | GCAAUUUCCAGCCCCAGGGCC | 13139 | GCAATTTCCAGCCCCAGGGCC |
| 7475 | GCACACAGUCCAGUUCGUGCA | 13140 | GCACACAGTCCAGTTCGTGCA |
| 7476 | GCACAGAUACUGGCAGCCGCC | 13141 | GCACAGATACTGGCAGCCGCC |
| 7477 | GCACAGCGGAAACCGGGGACU | 13142 | GCACAGCGGAAACCGGGGACT |
| 7478 | GCACAGGCACUCGUAGCCGAU | 13143 | GCACAGGCACTCGTAGCCGAT |
| 7479 | GCACCAGGCUGGUGUCCUGAC | 13144 | GCACCAGGCTGGTGTCCTGAC |
| 7480 | GCACCAUGCAUGGUUUUUUUU | 13145 | GCACCATGCATGGTTTTTTTT |
| 7481 | GCACCGGGCAGGAGGCCUCGU | 13146 | GCACCGGGCAGGAGGCCTCGT |
| 7482 | GCACCUCCAGAACUGAGGAAU | 13147 | GCACCTCCAGAACTGAGGAAT |
| 7483 | GCACCUGUGUCUGGCCAAAUU | 13148 | GCACCTGTGTCTGGCCAAATT |
| 7484 | GCACGAUGGGGAGGACAAUGG | 13149 | GCACGATGGGGAGGACAATGG |
| 7485 | GCACGCUCUGUCACACAGCUG | 13150 | GCACGCTCTGTCACACAGCTG |
| 7486 | GCACGUCUCCUGGGACUCAUC | 13151 | GCACGTCTCCTGGGACTCATC |
| 7487 | GCACGUCUUGGGGGAGCACGU | 13152 | GCACGTCTTGGGGGAGCACGT |
| 7488 | GCACGUCUUGGGGGGACAGCC | 13153 | GCACGTCTTGGGGGGACAGCC |
| 7489 | GCACGUGCCACCGUACCCAGC | 13154 | GCACGTGCCACCGTACCCAGC |
| 7490 | GCACUAGGUGGGGGACAUAA | 13155 | GCACTAGGTGGGCGGACATAA |
| 7491 | GCACUCAGCGCUGCCAUCGCA | 13156 | GCACTCAGCGCTGCCATCGCA |
| 7492 | GCACUCGCCACUUAGGCAGUG | 13157 | GCACTCGCCACTTAGGCAGTG |
| 7493 | GCACUCGUAGCCGAUCUUAAG | 13158 | GCACTCGTAGCCGATCTTAAG |
| 7494 | GCACUCUUUGAUGGGUUCAUC | 13159 | GCACTCTTTGATGGGTTCATC |
| 7495 | GCACUGAAAAUGGCUUCGUUG | 13160 | GCACTGAAAATGGCTTCGTTG |
| 7496 | GCACUGGAACUCGUUUCUUUC | 13161 | GCACTGGAACTCGTTTCTTTC |
| 7497 | GCACUGGAAGCUGGCGGGACC | 13162 | GCACTGGAAGCTGGCGGGACC |
| 7498 | GCACUGGAAUUCGUCAGGGCG | 13163 | GCACTGGAATTCGTCAGGGCG |
| 7499 | GCACUGGGGGGGACACAGUGU | 13164 | GCACTGGGGGGGACACAGTGT |
| 7500 | GCACUGUAGGGAGAGGCACGC | 13165 | GCACTGTAGGGAGAGGCACGC |
| 7501 | GCACUGUCCGAAGCCUGUUCU | 13166 | GCACTGTCCGAAGCCTGTTCT |
| 7502 | GCACUUAAUAAAUAUUAAGGG | 13167 | GCACTTAATAAATATTAAGGG |
| 7503 | GCACUUCCCAUCGUGGCAGCG | 13168 | GCACTTCCCATCGTGGCAGCG |
| 7504 | GCACUUGUAGCCACCCUCCAG | 13169 | GCACTTGTAGCCACCCTCCAG |
| 7505 | GCAGACCAAGGAGGGGCGGGG | 13170 | GCAGACCAAGGAGGGGCGGGG |
| 7506 | GCAGACCCACUUGUAGGAGAU | 13171 | GCAGACCCACTTGTAGGAGAT |
| 7507 | GCAGACGUGGGAACAGCCGCC | 13172 | GCAGACGTGGGAACAGCCGCC |
| 7508 | GCAGACUUUGUCCAGGGUGAU | 13173 | GCAGACTTTGTCCAGGGTGAT |
| 7509 | GCAGAGCUGGCUGCAGGUGUC | 13174 | GCAGAGCTGGCTGCAGGTGTC |
| 7510 | GCAGAGUGUCACAUUAACGCA | 13175 | GCAGAGTGTCACATTAACGCA |
| 7511 | GCAGAGUGUCAGACAGCCUUG | 13176 | GCAGAGTGTCAGACAGCCTTG |
| 7512 | GCAGAUCAUUCUCUGGGACAG | 13177 | GCAGATCATTCTCTGGGACAG |
| 7513 | GCAGAUGUUCACGCCACGUCA | 13178 | GCAGATGTTCACGCCACGTCA |
| 7514 | GCAGCAUGCCGUCCGGGCAGG | 13179 | GCAGCATGCCGTCCGGGCAGG |
| 7515 | GCAGCCAACUUCAUCGCUCAU | 13180 | GCAGCCAACTTCATCGCTCAT |
| 7516 | GCAGCCGCCAUUGCUCAGGGU | 13181 | GCAGCCGCCATTGCTCAGGGT |
| 7517 | GCAGCCGGGAGGUGUCGGGAA | 13182 | GCAGCCGGGAGGTGTCGGGAA |
| 7518 | GCAGCCUCAGCCUCUCUUGGC | 13183 | GCAGCCTCAGCCTCTCTTGGC |
| 7519 | GCAGCCUCAGCCUCUGUGAGG | 13184 | GCAGCCTCAGCCTCTGTGAGG |
| 7520 | GCAGCCUCUGCCAGGCAGUGU | 13185 | GCAGCCTCTGCCAGGCAGTGT |
| 7521 | GCAGCGAAACUCGUCCUGGGA | 13186 | GCAGCGAAACTCGTCCTGGGA |
| 7522 | GCAGCGGUUGACACGGCCCCC | 13187 | GCAGCGGTTGACACGGCCCCC |
| 7523 | GCAGCUCCUCAUGUCCCUGGC | 13188 | GCAGCTCCTCATGTCCCTGGC |
| 7524 | GCAGGAGGCCUCGUCUGAGCC | 13189 | GCAGGAGGCCTCGTCTGAGCC |
| 7525 | GCAGGCAAUGCUUUGGUCUUC | 13190 | GCAGGCAATGCTTTGGTCTTC |
| 7526 | GCAGGCCACGUGUCCUCACGG | 13191 | GCAGGCCACGTGTCCTCACGG |
| 7527 | GCAGGCCCACAGCUGGGGGAU | 13192 | GCAGGCCCACAGCTGGGGGAT |
| 7528 | GCAGGCCUUCGUGUGGGGGUC | 13193 | GCAGGCCTTCGTGTGGGGGTC |
| 7529 | GCAGGCGCAGGUAAACUUGGG | 13194 | GCAGGCGCAGGTAAACTTGGG |
| 7530 | GCAGGGACGGGACUCCAGGCA | 13195 | GCAGGGACGGGACTCCAGGCA |
| 7531 | GCAGGGAGGCACAGAUACUGG | 13196 | GCAGGGAGGCACAGATACTGG |
| 7532 | GCAGGGCCAGGGGGUCCCGUU | 13197 | GCAGGGCCAGGGGGTCCCGTT |
| 7533 | GCAGGGGCUACUGUCCCCUUG | 13198 | GCAGGGGCTACTGTCCCCTTG |
| 7534 | GCAGGUAAACUUGGGCGAGUG | 13199 | GCAGGTAAACTTGGGCGAGTG |
| 7535 | GCAGGUGACAGACAAGCACGU | 13200 | GCAGGTGACAGACAAGCACGT |
| 7536 | GCAGGUGGAGCUGUUGCACUG | 13201 | GCAGGTGGAGCTGTTGCACTG |
| 7537 | GCAGGUGUCGGGAUCCUGACA | 13202 | GCAGGTGTCGGGATCCTGACA |
| 7538 | GCAGUCAUAUUCCCGGUCACA | 13203 | GCAGTCATATTCCCGGTCACA |
| 7539 | GCAGUCCACUUGGCCAUCGCA | 13204 | GCAGTCCACTTGGCCATCGCA |
| 7540 | GCAGUCCCCGCCGCGGCGAGG | 13205 | GCAGTCCCCGCCGCGGCGAGG |
| 7541 | GCAGUCCCGGUCUGAGUCACA | 13206 | GCAGTCCCGGTCTGAGTCACA |
| 7542 | GCAGUCGGGGCCACCAUCACA | 13207 | GCAGTCGGGGCCACCATCACA |
| 7543 | GCAGUCGGGGUCGUUGUCGCA | 13208 | GCAGTCGGGGTCGTTGTCGCA |
| 7544 | GCAGUCUCUAGCCAUGUUGCA | 13209 | GCAGTCTCTAGCCATGTTGCA |
| 7545 | GCAGUGGAACUCGAAGGCCGA | 13210 | GCAGTGGAACTCGAAGGCCGA |
| 7546 | GCAGUGGCAUAAUCAUAGCUC | 13211 | GCAGTGGCATAATCATAGCTC |
| 7547 | GCAGUGGCGCAAUCUCGGCUC | 13212 | GCAGTGGCGCAATCTCGGCTC |
| 7548 | GCAGUGGCUCGAUCAUGGCUC | 13213 | GCAGTGGCTCGATCATGGCTC |
| 7549 | GCAGUGUCCCGACCCGGAUCA | 13214 | GCAGTGTCCCGACCCGGATCA |
| 7550 | GCAGUUCUCGAUAUUCACGAU | 13215 | GCAGTTCTCGATATTCACGAT |
| 7551 | GCAGUUUCCAUCAGAGCACUG | 13216 | GCAGTTTCCATCAGAGCACTG |
| 7552 | GCAGUUUUCCUCGUCAGAUUU | 13217 | GCAGTTTTCCTCGTCAGATTT |
| 7553 | GCAUAAUCAUAGCUCACGACA | 13218 | GCATAATCATAGCTCACGACA |
| 7554 | GCAUAGAAGAGGUAAACACGU | 13219 | GCATAGAAGAGGTAAACACGT |
| 7555 | GCAUAGAAGUGGAAGCACUAG | 13220 | GCATAGAAGTGGAAGCACTAG |
| 7556 | GCAUCAUUUGGUGAAUGAGUU | 13221 | GCATCATTTGGTGAATGAGTT |
| 7557 | GCAUCUGUCGCCCACGUGGCU | 13222 | GCATCTGTCGCCCACGTGGCT |
| 7558 | GCAUCUGUCGCCCACUGCAGU | 13223 | GCATCTGTCGCCCACTGCAGT |
| 7559 | GCAUCUUCGCUGGGCCACCAG | 13224 | GCATCTTCGCTGGGCCACCAG |
| 7560 | GCAUGCCGUCCGGGCAGGCGC | 13225 | GCATGCCGTCCGGGCAGGCGC |
| 7561 | GCAUGGGCACUGUCCGAAGCC | 13226 | GCATGGGCACTGTCCGAAGCC |
| 7562 | GCAUGGUUUUUUUUUGUUUUU | 13227 | GCATGGTTTTTTTTTGTTTTT |
| 7563 | GCAUGUUUUAAACACAUACCC | 13228 | GCATGTTTTAAACACATACCC |
| 7564 | GCAUUCAUUGACACGGGCUUU | 13229 | GCATTCATTGACACGGGCTTT |
| 7565 | GCAUUCGCCGCUGUGACACUU | 13230 | GCATTCGCCGCTGTGACACTT |
| 7566 | GCAUUCGUUGGUCCCGCACUC | 13231 | GCATTCGTTGGTCCCGCACTC |
| 7567 | GCAUUUACGUGCUCCGAAACC | 13232 | GCATTTACGTGCTCCGAAACC |
| 7568 | GCAUUUCAGAGGCAAUAACCC | 13233 | GCATTTCAGAGGCAATAACCC |
| 7569 | GCAUUUCCCGUCUUGGCACUG | 13234 | GCATTTCCCGTCTTGGCACTG |
| 7570 | GCAUUUGCAUAGAAGUGGAAG | 13235 | GCATTTGCATAGAAGTGGAAG |
| 7571 | GCCAAAUGUACACAGUGUACA | 13236 | GCCAAATGTACACAGTGTACA |
| 7572 | GCCAAAUUCAUUUUUUAAGAG | 13237 | GCCAAATTCATTTTTTAAGAG |
| 7573 | GCCAACAAGUUGACAUCGGAA | 13238 | GCCAACAAGTTGACATCGGAA |
| 7574 | GCCAACUUCAUCGCUCAUGUC | 13239 | GCCAACTTCATCGCTCATGTC |
| 7575 | GCCAAGGAGAAGGGGUGGGCC | 13240 | GCCAAGGAGAAGGGGTGGGCC |
| 7576 | GCCAAGGUAACCGGGUGUCUC | 13241 | GCCAAGGTAACCGGGTGTCTC |
| 7577 | GCCAAUCCCUUGUGACAUCUU | 13242 | GCCAATCCCTTGTGACATCTT |
| 7578 | GCCACACCUGGGUGCAGGCCA | 13243 | GCCACACCTGGGTGCAGGCCA |
| 7579 | GCCACAGAGCACAGCGGAAAC | 13244 | GCCACAGAGCACAGCGGAAAC |
| 7580 | GCCACAGCGCAGUUUUCCUCG | 13245 | GCCACAGCGCAGTTTTCCTCG |
| 7581 | GCCACAGGACAGCCUUGCUCG | 13246 | GCCACAGGACAGCCTTGCTCG |
| 7582 | GCCACCACGCCCGGCUAAUUU | 13247 | GCCACCACGCCCGGCTAATTT |
| 7583 | GCCACCACGGAUUCAGCCAGA | 13248 | GCCACCACGGATTCAGCCAGA |
| 7584 | GCCACCAGCUGGAAGCCGUCG | 13249 | GCCACCAGCTGGAAGCCGTCG |
| 7585 | GCCACCAUCACAGCGCCAGCU | 13250 | GCCACCATCACAGCGCCAGCT |
| 7586 | GCCACCCUCCAGGUUCACGCA | 13251 | GCCACCCTCCAGGTTCACGCA |
| 7587 | GCCACCGUACCCAGCUGAUUU | 13252 | GCCACCGTACCCAGCTGATTT |
| 7588 | GCCACCUCCGUGUCCAGAGCG | 13253 | GCCACCTCCGTGTCCAGAGCG |
| 7589 | GCCACGUCAUCCUCCAGACUG | 13254 | GCCACGTCATCCTCCAGACTG |
| 7590 | GCCACGUGUCCUCACGGCCAA | 13255 | GCCACGTGTCCTCACGGCCAA |
| 7591 | GCCACUCAUACAUACAACGGG | 13256 | GCCACTCATACATACAACGGG |
| 7592 | GCCACUCAUCCGAGCCAUCUU | 13257 | GCCACTCATCCGAGCCATCTT |
| 7593 | GCCACUGAAUGUUUUCAGUCA | 13258 | GCCACTGAATGTTTTCAGTCA |
| 7594 | GCCACUGAGGAGAUCUAGGGU | 13259 | GCCACTGAGGAGATCTAGGGT |
| 7595 | GCCACUGCAGCCUCAGCCUCU | 13260 | GCCACTGCAGCCTCAGCCTCT |
| 7596 | GCCACUGCGCCCGGCCUCACC | 13261 | GCCACTGCGCCCGGCCTCACC |
| 7597 | GCCACUUAGGCAGUGGAACUC | 13262 | GCCACTTAGGCAGTGGAACTC |
| 7598 | GCCACUUGCUUCUCUGGGCCU | 13263 | GCCACTTGCTTCTCTGGGCCT |
| 7599 | GCCAGACUGCGGACUGCAGUG | 13264 | GCCAGACTGCGGACTGCAGTG |
| 7600 | GCCAGAGGCCAUGGGCUGCUG | 13265 | GCCAGAGGCCATGGGCTGCTG |
| 7601 | GCCAGAUCAUUUCCGACGCCA | 13266 | GCCAGATCATTTCCGACGCCA |
| 7602 | GCCAGCAACGUCGCCCAGAGC | 13267 | GCCAGCAACGTCGCCCAGAGC |
| 7603 | GCCAGCAGCAUGCCGUCCGGG | 13268 | GCCAGCAGCATGCCGTCCGGG |
| 7604 | GCCAGCCCGUCGGGGGCCUGG | 13269 | GCCAGCCCGTCGGGGGCCTGG |
| 7605 | GCCAGCCUCUUUUCAUCCUCC | 13270 | GCCAGCCTCTTTTCATCCTCC |
| 7606 | GCCAGCUGGAGUGGAUGCACU | 13271 | GCCAGCTGGAGTGGATGCACT |
| 7607 | GCCAGGAGGGCCCAGGAUAAC | 13272 | GCCAGGAGGGCCCAGGATAAC |
| 7608 | GCCAGGAUGGUCUCGAUCUCC | 13273 | GCCAGGATGGTCTCGATCTCC |
| 7609 | GCCAGGCAGUGUCCCGACCCG | 13274 | GCCAGGCAGTGTCCCGACCCG |
| 7610 | GCCAGGGGGUCCCGUUGGUGC | 13275 | GCCAGGGGGTCCCGTTGGTGC |
| 7611 | GCCAGUUCUUCCAUAGAAGGA | 13276 | GCCAGTTCTTCCATAGAAGGA |
| 7612 | GCCAUCCUGGCACUCAGCGCU | 13277 | GCCATCCTGGCACTCAGCGCT |
| 7613 | GCCAUCGCACCUCCAGAACUG | 13278 | GCCATCGCACCTCCAGAACTG |
| 7614 | GCCAUCGCAGACCCACUUGUA | 13279 | GCCATCGCAGACCCACTTGTA |
| 7615 | GCCAUCUGCUGUUGUGUGUUG | 13280 | GCCATCTGCTGTTGTGTGTTG |
| 7616 | GCCAUCUUCGCAGUCGGGGUC | 13281 | GCCATCTTCGCAGTCGGGGTC |
| 7617 | GCCAUGAACAGGAUCCACCAC | 13282 | GCCATGAACAGGATCCACCAC |
| 7618 | GCCAUGAAUCCCUUUCCUGGC | 13283 | GCCATGAATCCCTTTCCTGGC |
| 7619 | GCCAUGCCUGGCCAGAGGCCA | 13284 | GCCATGCCTGGCCAGAGGCCA |
| 7620 | GCCAUGGAUGCAGUUUCCAUC | 13285 | GCCATGGATGCAGTTTCCATC |
| 7621 | GCCAUGGGCUGCUGAUGCACC | 13286 | GCCATGGGCTGCTGATGCACC |
| 7622 | GCCAUGUUGCAGACUUUGUCC | 13287 | GCCATGTTGCAGACTTTGTCC |
| 7623 | GCCAUUGCAUGGGCACUGUCC | 13288 | GCCATTGCATGGGCACTGTCC |
| 7624 | GCCAUUGCUCAGGGUGGUCCU | 13289 | GCCATTGCTCAGGGTGGTCCT |
| 7625 | GCCAUUGGGCCACUGAAUGUU | 13290 | GCCATTGGGCCACTGAATGTT |
| 7626 | GCCCACAGCCUUGCAGGCCUU | 13291 | GCCCACAGCCTTGCAGGCCTT |
| 7627 | GCCCACAGCUGGGGGAUGCAG | 13292 | GCCCACAGCTGGGGGATGCAG |
| 7628 | GCCCACGUGGCUGAAGAUCUC | 13293 | GCCCACGTGGCTGAAGATCTC |
| 7629 | GCCCACUGCAGUCCCCGCCGC | 13294 | GCCCACTGCAGTCCCCGCCGC |
| 7630 | GCCCAGAGCUUGGUGAGACAU | 13295 | GCCCAGAGCTTGGTGAGACAT |
| 7631 | GCCCAGGACAGAGUCGGUCCA | 13296 | GCCCAGGACAGAGTCGGTCCA |
| 7632 | GCCCAGGAUAACGGAGGGCGA | 13297 | GCCCAGGATAACGGAGGGCGA |
| 7633 | GCCCAGGCCAGACUGCGGACU | 13298 | GCCCAGGCCAGACTGCGGACT |
| 7634 | GCCCAGGCUGGAAAGCAGUGG | 13299 | GCCCAGGCTGGAAAGCAGTGG |
| 7635 | GCCCAGGCUGGAGUGCAGUGG | 13300 | GCCCAGGCTGGAGTGCAGTGG |
| 7636 | GCCCAGGGGUGGCCCCAGGCA | 13301 | GCCCAGGGGTGGCCCCAGGCA |
| 7637 | GCCCCAGGCAGCCGGGAGGUG | 13302 | GCCCCAGGCAGCCGGGAGGTG |
| 7638 | GCCCCAGGGCCCCAUGCUCGC | 13303 | GCCCCAGGGCCCCATGCTCGC |
| 7639 | GCCCCAUGCUCGCAGCCUCUG | 13304 | GCCCCATGCTCGCAGCCTCTG |
| 7640 | GCCCCCACAGCUGAAGUCCCC | 13305 | GCCCCCACAGCTGAAGTCCCC |
| 7641 | GCCCCCCAGGACAAGGUGGGG | 13306 | GCCCCCCAGGACAAGGTGGGG |
| 7642 | GCCCCCGAGACCAGAGACCCA | 13307 | GCCCCCGAGACCAGAGACCCA |
| 7643 | GCCCCCGUUGACAUCGAUGCU | 13308 | GCCCCCGTTGACATCGATGCT |
| 7644 | GCCCCCUUUCUUGAUCUUGGC | 13309 | GCCCCCTTTCTTGATCTTGGC |
| 7645 | GCCCCGAGAAUAGUCACUGAA | 13310 | GCCCCGAGAATAGTCACTGAA |
| 7646 | GCCCCUCUCUGUCCCCGGCAU | 13311 | GCCCCTCTCTGTCCCCGGCAT |
| 7647 | GCCCCUCUGUGAACUUGAUCG | 13312 | GCCCCTCTGTGAACTTGATCG |
| 7648 | GCCCGCCACCACGCCCGGCUA | 13313 | GCCCGCCACCACGCCCGGCTA |
| 7649 | GCCCGGCCUCACCGUGCAUGU | 13314 | GCCCGGCCTCACCGTGCATGT |
| 7650 | GCCCGGCUAAUUUUUUGUAUU | 13315 | GCCCGGCTAATTTTTTGTATT |
| 7651 | GCCCGUCGGGGGCCUGGAUGU | 13316 | GCCCGTCGGGGGCCTGGATGT |
| 7652 | GCCCUCACGCUACUGGGCUUC | 13317 | GCCCTCACGCTACTGGGCTTC |
| 7653 | GCCCUUGGCUUGGAGCCGUUC | 13318 | GCCCTTGGCTTGGAGCCGTTC |
| 7654 | GCCCUUGGUAUCCGCAACAGA | 13319 | GCCCTTGGTATCCGCAACAGA |
| 7655 | GCCGAGAGAUGCACUUCCCAU | 13320 | GCCGAGAGATGCACTTCCCAT |
| 7656 | GCCGAGCAGGGGCUACUGUCC | 13321 | GCCGAGCAGGGGCTACTGTCC |
| 7657 | GCCGAUCUUAAGGUCAUUGCA | 13322 | GCCGATCTTAAGGTCATTGCA |
| 7658 | GCCGCCAGUUCUUCCAUAGAA | 13323 | GCCGCCAGTTCTTCCATAGAA |
| 7659 | GCCGCCAUUGCUCAGGGUGGU | 13324 | GCCGCCATTGCTCAGGGTGGT |
| 7660 | GCCGCCGUUGUUGUCCAAGCA | 13325 | GCCGCCGTTGTTGTCCAAGCA |
| 7661 | GCCGCGGCGAGGAGCAAGGCG | 13326 | GCCGCGGCGAGGAGCAAGGCG |
| 7662 | GCCGCUGUGACACUUGAACUU | 13327 | GCCGCTGTGACACTTGAACTT |
| 7663 | GCCGGCGAGGUCUCAGGAAGG | 13328 | GCCGGCGAGGTCTCAGGAAGG |
| 7664 | GCCGGCUGCCAUGGAUGCAGU | 13329 | GCCGGCTGCCATGGATGCAGT |
| 7665 | GCCGGGAGGUGUCGGGAACAG | 13330 | GCCGGGAGGTGTCGGGAACAG |
| 7666 | GCCGGGGGCCCCUCUGUGAAC | 13331 | GCCGGGGGCCCCTCTGTGAAC |
| 7667 | GCCGGUUGGUGAAGAAGAGGU | 13332 | GCCGGTTGGTGAAGAAGAGGT |
| 7668 | GCCGUCCAAGCAGUCCCGGUC | 13333 | GCCGTCCAAGCAGTCCCGGTC |
| 7669 | GCCGUCCGGGCAGGCGCAGGU | 13334 | GCCGTCCGGGCAGGCGCAGGT |
| 7670 | GCCGUCCUGGUUGUGGCAAAU | 13335 | GCCGTCCTGGTTGTGGCAAAT |
| 7671 | GCCGUCGGGGCACAGGCACUC | 13336 | GCCGTCGGGGCACAGGCACTC |
| 7672 | GCCGUGGGCUCUGUCAAGCUG | 13337 | GCCGTGGGCTCTGTCAAGCTG |
| 7673 | GCCGUUCUCCCUGAAUAACGU | 13338 | GCCGTTCTCCCTGAATAACGT |
| 7674 | GCCGUUGUCGCAGUCCACUUG | 13339 | GCCGTTGTCGCAGTCCACTTG |
| 7675 | GCCGUUGUUGUCCAAGCAUUC | 13340 | GCCGTTGTTGTCCAAGCATTC |
| 7676 | GCCUCAAAUGCCUGAGCUCAA | 13341 | GCCTCAAATGCCTGAGCTCAA |
| 7677 | GCCUCACCGUGCAUGUUUUAA | 13342 | GCCTCACCGTGCATGTTTTAA |
| 7678 | GCCUCAGCCUCCCAAGUAGCU | 13343 | GCCTCAGCCTCCCAAGTAGCT |
| 7679 | GCCUCAGCCUCCCGAGUAGCU | 13344 | GCCTCAGCCTCCCGAGTAGCT |
| 7680 | GCCUCAGCCUCUCUUGGCUGG | 13345 | GCCTCAGCCTCTCTTGGCTGG |
| 7681 | GCCUCAGCCUCUGUGAGGCAG | 13346 | GCCTCAGCCTCTGTGAGGCAG |
| 7682 | GCCUCCCAAAGUGCUGGGAUU | 13347 | GCCTCCCAAAGTGCTGGGATT |
| 7683 | GCCUCCCAAGUAGCUGGGAUC | 13348 | GCCTCCCAAGTAGCTGGGATC |
| 7684 | GCCUCCCAGAUGAAUAAAUAU | 13349 | GCCTCCCAGATGAATAAATAT |
| 7685 | GCCUCCCGAGUAGCUGGGACU | 13350 | GCCTCCCGAGTAGCTGGGACT |
| 7686 | GCCUCGCCAUGCCUGGCCAGA | 13351 | GCCTCGCCATGCCTGGCCAGA |
| 7687 | GCCUCGGCCUCCCAAAGUGCU | 13352 | GCCTCGGCCTCCCAAAGTGCT |
| 7688 | GCCUCGUCUGAGCCGUCCAAG | 13353 | GCCTCGTCTGAGCCGTCCAAG |
| 7689 | GCCUCUCUUGGCUGGGUGAGG | 13354 | GCCTCTCTTGGCTGGGTGAGG |
| 7690 | GCCUCUGCCAGGCAGUGUCCC | 13355 | GCCTCTGCCAGGCAGTGTCCC |
| 7691 | GCCUCUGGGAAGUGGCAUCAU | 13356 | GCCTCTGGGAAGTGGCATCAT |
| 7692 | GCCUCUGUGAGGCAGCUCCUC | 13357 | GCCTCTGTGAGGCAGCTCCTC |
| 7693 | GCCUCUUUUCAUCCUCCAAGA | 13358 | GCCTCTTTTCATCCTCCAAGA |
| 7694 | GCCUGACAUCCCCUCCCCUUU | 13359 | GCCTGACATCCCCTCCCCTTT |
| 7695 | GCCUGACGGUGGAUGUCUCCU | 13360 | GCCTGACGGTGGATGTCTCCT |
| 7696 | GCCUGAGCUCAAACCAUCCUC | 13361 | GCCTGAGCTCAAACCATCCTC |
| 7697 | GCCUGGAUGUCUCUGCUGAUG | 13362 | GCCTGGATGTCTCTGCTGATG |
| 7698 | GCCUGGCCAGAGGCCAUGGGC | 13363 | GCCTGGCCAGAGGCCATGGGC |
| 7699 | GCCUGGGAUCCCAUCCCAACA | 13364 | GCCTGGGATCCCATCCCAACA |
| 7700 | GCCUGGUCUCCAACUCCUGAA | 13365 | GCCTGGTCTCCAACTCCTGAA |
| 7701 | GCCUGUUCUGCCUCCCAGAUG | 13366 | GCCTGTTCTGCCTCCCAGATG |
| 7702 | GCCUGUUUCUCUUAUCCUUCA | 13367 | GCCTGTTTCTCTTATCCTTCA |
| 7703 | GCCUUCCUCACACUGGCACUU | 13368 | GCCTTCCTCACACTGGCACTT |
| 7704 | GCCUUCGUGUGGGGGUCCAGC | 13369 | GCCTTCGTGTGGGGGTCCAGC |
| 7705 | GCCUUGAGGGUGGCCGGGGGC | 13370 | GCCTTGAGGGTGGCCGGGGGC |
| 7706 | GCCUUGCAGGCCUUCGUGUGG | 13371 | GCCTTGCAGGCCTTCGTGTGG |
| 7707 | GCCUUGCUCGUCUGAGCCGUU | 13372 | GCCTTGCTCGTCTGAGCCGTT |
| 7708 | GCCUUUGUGACAGGAACCGCG | 13373 | GCCTTTGTGACAGGAACCGCG |
| 7709 | GCGAAACUCGUCCUGGGAGCA | 13374 | GCGAAACTCGTCCTGGGAGCA |
| 7710 | GCGACAGGUGGCCACAGCGCA | 13375 | GCGACAGGTGGCCACAGCGCA |
| 7711 | GCGACAGGUGGCCACAGGACA | 13376 | GCGACAGGTGGCCACAGGACA |
| 7712 | GCGACCACGUUCCUCAGGUUG | 13377 | GCGACCACGTTCCTCAGGTTG |
| 7713 | GCGACGGUCCAGCGCAAUUUC | 13378 | GCGACGGTCCAGCGCAATTTC |
| 7714 | GCGACUCGUGUAUGUCGCGGA | 13379 | GCGACTCGTGTATGTCGCGGA |
| 7715 | GCGAGGAGCAAGGCGACGGUC | 13380 | GCGAGGAGCAAGGCGACGGTC |
| 7716 | GCGAGGGCGCCCCCCAGGACA | 13381 | GCGAGGGCGCCCCCCAGGACA |
| 7717 | GCGAGGUCUCAGGAAGGGUUC | 13382 | GCGAGGTCTCAGGAAGGGTTC |
| 7718 | GCGAGUAGAUGUCCACACCAU | 13383 | GCGAGTAGATGTCCACACCAT |
| 7719 | GCGAGUGGGGGUUGAUCUGCG | 13384 | GCGAGTGGGGGTTGATCTGCG |
| 7720 | GCGAUCCUCCUGCCUCAGCCU | 13385 | GCGATCCTCCTGCCTCAGCCT |
| 7721 | GCGAUGGAGCCCACAGCCUUG | 13386 | GCGATGGAGCCCACAGCCTTG |
| 7722 | GCGAUGGGGAGGACAAUGGAC | 13387 | GCGATGGGGAGGACAATGGAC |
| 7723 | GCGAUUUCAGAAAGAACCUGA | 13388 | GCGATTTCAGAAAGAACCTGA |
| 7724 | GCGCAAUCUCGGCUCACUGCA | 13389 | GCGCAATCTCGGCTCACTGCA |
| 7725 | GCGCAAUUUCCAGCCCCAGGG | 13390 | GCGCAATTTCCAGCCCCAGGG |
| 7726 | GCGCAGGUAAACUUGGGCGAG | 13391 | GCGCAGGTAAACTTGGGCGAG |
| 7727 | GCGCAGUUUUCCUCGUCAGAU | 13392 | GCGCAGTTTTCCTCGTCAGAT |
| 7728 | GCGCCAGCUGGAGUGGAUGCA | 13393 | GCGCCAGCTGGAGTGGATGCA |
| 7729 | GCGCCCCCCAGGACAAGGUGG | 13394 | GCGCCCCCCAGGACAAGGTGG |
| 7730 | GCGCCCGGCCUCACCGUGCAU | 13395 | GCGCCCGGCCTCACCGTGCAT |
| 7731 | GCGCUGCCAUCGCAGACCCAC | 13396 | GCGCTGCCATCGCAGACCCAC |
| 7732 | GCGCUGCGGCCACUCAUCCGA | 13397 | GCGCTGCGGCCACTCATCCGA |
| 7733 | GCGGAAACCGGGGACUUCGCC | 13398 | GCGGAAACCGGGGACTTCGCC |
| 7734 | GCGGACAUAAAGACGACAAUG | 13399 | GCGGACATAAAGACGACAATG |
| 7735 | GCGGACUGCAGUGGCGCAAUC | 13400 | GCGGACTGCAGTGGCGCAATC |
| 7736 | GCGGAGGAACGCCAGGCCUCC | 13401 | GCGGAGGAACGCCAGGCCTCC |
| 7737 | GCGGAGGCGGCUCCGGGGACC | 13402 | GCGGAGGCGGCTCCGGGGACC |
| 7738 | GCGGCCACUCAUCCGAGCCAU | 13403 | GCGGCCACTCATCCGAGCCAT |
| 7739 | GCGGCCACUGAGGAGAUCUAG | 13404 | GCGGCCACTGAGGAGATCTAG |
| 7740 | GCGGCGAGGAGCAAGGCGACG | 13405 | GCGGCGAGGAGCAAGGCGACG |
| 7741 | GCGGCUCCGGGGACCCGGGAC | 13406 | GCGGCTCCGGGGACCCGGGAC |
| 7742 | GCGGGACCACAGGUGAGCACC | 13407 | GCGGGACCACAGGTGAGCACC |
| 7743 | GCGGGAGUCUGACGGAGGCUG | 13408 | GCGGGAGTCTGACGGAGGCTG |
| 7744 | GCGGGAGUUCCCCAGUCAGUC | 13409 | GCGGGAGTTCCCCAGTCAGTC |
| 7745 | GCGGGGAGAUUCUGAGGCAAC | 13410 | GCGGGGAGATTCTGAGGCAAC |
| 7746 | GCGGGGCAGGGAGGCACAGAU | 13411 | GCGGGGCAGGGAGGCACAGAT |
| 7747 | GCGGUUGACACGGCCCCCACA | 13412 | GCGGTTGACACGGCCCCCACA |
| 7748 | GCGGUUGGCACUGAAAAUGGC | 13413 | GCGGTTGGCACTGAAAATGGC |
| 7749 | GCGUCAUCUUCCUGACCUCGU | 13414 | GCGTCATCTTCCTGACCTCGT |
| 7750 | GCGUGAGCCACUGCGCCCGGC | 13415 | GCGTGAGCCACTGCGCCCGGC |
| 7751 | GCGUGAGGCACCUGUGUCUGG | 13416 | GCGTGAGGCACCTGTGTCTGG |
| 7752 | GCGUUGCACACAGUCCAGUUC | 13417 | GCGTTGCACACAGTCCAGTTC |
| 7753 | GCUAACCUGGCUGUCUAGCAA | 13418 | GCTAACCTGGCTGTCTAGCAA |
| 7754 | GCUAAUUUUUUGUAUUUUUAG | 13419 | GCTAATTTTTTGTATTTTTAG |
| 7755 | GCUACAAAAACCUUCUAUACA | 13420 | GCTACAAAAACCTTCTATACA |
| 7756 | GCUACUGGGCUUCUUCUCAUU | 13421 | GCTACTGGGCTTCTTCTCATT |
| 7757 | GCUACUGUCCCCUUGGAACAC | 13422 | GCTACTGTCCCCTTGGAACAC |
| 7758 | GCUCAAACCAUCCUCCCACCU | 13423 | GCTCAAACCATCCTCCCACCT |
| 7759 | GCUCACGACAGCCUCAAAUGC | 13424 | GCTCACGACAGCCTCAAATGC |
| 7760 | GCUCACUGCAACCUCCACCUC | 13425 | GCTCACTGCAACCTCCACCTC |
| 7761 | GCUCACUGCAAGCUCCGCUUC | 13426 | GCTCACTGCAAGCTCCGCTTC |
| 7762 | GCUCAGCCUCCCAAAGUGCUG | 13427 | GCTCAGCCTCCCAAAGTGCTG |
| 7763 | GCUCAGGCCUGGUCUCCAACU | 13428 | GCTCAGGCCTGGTCTCCAACT |
| 7764 | GCUCAGGCGAUCCUCCUGCCU | 13429 | GCTCAGGCGATCCTCCTGCCT |
| 7765 | GCUCAGGGUGGUCCUCUCACA | 13430 | GCTCAGGGTGGTCCTCTCACA |
| 7766 | GCUCAUGUCCUUGCAGUCAUA | 13431 | GCTCATGTCCTTGCAGTCATA |
| 7767 | GCUCCGAAACCAGAAAGGCUU | 13432 | GCTCCGAAACCAGAAAGGCTT |
| 7768 | GCUCCGCUUCCCGGGUUCACA | 13433 | GCTCCGCTTCCCGGGTTCACA |
| 7769 | GCUCCGGGGACCCGGGACUUG | 13434 | GCTCCGGGGACCCGGGACTTG |
| 7770 | GCUCCGGUCCAGCGUCAUCUU | 13435 | GCTCCGGTCCAGCGTCATCTT |
| 7771 | GCUCCGUGGCGAUGGGGAGGA | 13436 | GCTCCGTGGCGATGGGGAGGA |
| 7772 | GCUCCUCAUGUCCCUGGCCAG | 13437 | GCTCCTCATGTCCCTGGCCAG |
| 7773 | GCUCGAUCAUGGCUCACUGCA | 13438 | GCTCGATCATGGCTCACTGCA |
| 7774 | GCUCGCAGCCUCUGCCAGGCA | 13439 | GCTCGCAGCCTCTGCCAGGCA |
| 7775 | GCUCGUCUGAGCCGUUGUCGC | 13440 | GCTCGTCTGAGCCGTTGTCGC |
| 7776 | GCUCUGCCUCUGGGAAGUGGC | 13441 | GCTCTGCCTCTGGGAAGTGGC |
| 7777 | GCUCUGGCAGGCAAUGCUUUG | 13442 | GCTCTGGCAGGCAATGCTTTG |
| 7778 | GCUCUGUCAAGCUGGGUGCUG | 13443 | GCTCTGTCAAGCTGGGTGCTG |
| 7779 | GCUCUGUCACACAGCUGCCCC | 13444 | GCTCTGTCACACAGCTGCCCC |
| 7780 | GCUCUGUCGCCCAGGCCAGAC | 13445 | GCTCTGTCGCCCAGGCCAGAC |
| 7781 | GCUGAAGAUCUCGGCUGCAGA | 13446 | GCTGAAGATCTCGGCTGCAGA |
| 7782 | GCUGAAGUCCCCGGAUUUGCA | 13447 | GCTGAAGTCCCCGGATTTGCA |
| 7783 | GCUGACCUUUAGCCUGACGGU | 13448 | GCTGACCTTTAGCCTGACGGT |
| 7784 | GCUGAUGACGGUGUCAUAGGA | 13449 | GCTGATGACGGTGTCATAGGA |
| 7785 | GCUGAUGCACCAUGCAUGGUU | 13450 | GCTGATGCACCATGCATGGTT |
| 7786 | GCUGAUUUUUAACUUUUUGGU | 13451 | GCTGATTTTTAACTTTTTGGT |
| 7787 | GCUGCAGACCAAGGAGGGGCG | 13452 | GCTGCAGACCAAGGAGGGGCG |
| 7788 | GCUGCAGAUCAUUCUCUGGGA | 13453 | GCTGCAGATCATTCTCTGGGA |
| 7789 | GCUGCAGGUGUCGGGAUCCUG | 13454 | GCTGCAGGTGTCGGGATCCTG |
| 7790 | GCUGCCAUCGCAGACCCACUU | 13455 | GCTGCCATCGCAGACCCACTT |
| 7791 | GCUGCCAUGGAUGCAGUUUCC | 13456 | GCTGCCATGGATGCAGTTTCC |
| 7792 | GCUGCCCCCGAGACCAGAGAC | 13457 | GCTGCCCCCGAGACCAGAGAC |
| 7793 | GCUGCGGCCACUCAUCCGAGC | 13458 | GCTGCGGCCACTCATCCGAGC |
| 7794 | GCUGCUGAUGCACCAUGCAUG | 13459 | GCTGCTGATGCACCATGCATG |
| 7795 | GCUGGAAACCCUGGCUUCCCG | 13460 | GCTGGAAACCCTGGCTTCCCG |
| 7796 | GCUGGAAAGCAGUGGCAUAAU | 13461 | GCTGGAAAGCAGTGGCATAAT |
| 7797 | GCUGGAAGCCGUCGGGGCACA | 13462 | GCTGGAAGCCGTCGGGGCACA |
| 7798 | GCUGGAAGCCUUCCUCACACU | 13463 | GCTGGAAGCCTTCCTCACACT |
| 7799 | GCUGGAGUGCAGUGGCUCGAU | 13464 | GCTGGAGTGCAGTGGCTCGAT |
| 7800 | GCUGGAGUGGAUGCACUCGCC | 13465 | GCTGGAGTGGATGCACTCGCC |
| 7801 | GCUGGCCACCUCCGUGUCCAG | 13466 | GCTGGCCACCTCCGTGTCCAG |
| 7802 | GCUGGCGGGACCACAGGUGAG | 13467 | GCTGGCGGGACCACAGGTGAG |
| 7803 | GCUGGCUGCAGGUGUCGGGAU | 13468 | GCTGGCTGCAGGTGTCGGGAT |
| 7804 | GCUGGGACUACAGGUGCCCGC | 13469 | GCTGGGACTACAGGTGCCCGC |
| 7805 | GCUGGGAUCACAGGCACGUGC | 13470 | GCTGGGATCACAGGCACGTGC |
| 7806 | GCUGGGAUUACAGGCGUGAGC | 13471 | GCTGGGATTACAGGCGTGAGC |
| 7807 | GCUGGGAUUACAGGCGUGAGG | 13472 | GCTGGGATTACAGGCGTGAGG |
| 7808 | GCUGGGCCACCAGCUGGAAGC | 13473 | GCTGGGCCACCAGCTGGAAGC |
| 7809 | GCUGGGGGAUGCAGGUGGAGC | 13474 | GCTGGGGGATGCAGGTGGAGC |
| 7810 | GCUGGGUGAGGUUGUGGAAGA | 13475 | GCTGGGTGAGGTTGTGGAAGA |
| 7811 | GCUGGGUGCUGCAGAUCAUUC | 13476 | GCTGGGTGCTGCAGATCATTC |
| 7812 | GCUGGUGGGGCGAGGGCGCCC | 13477 | GCTGGTGGGGCGAGGGCGCCC |
| 7813 | GCUGGUGUACUCGCUCCGGUC | 13478 | GCTGGTGTACTCGCTCCGGTC |
| 7814 | GCUGGUGUCCUGACAGCCACA | 13479 | GCTGGTGTCCTGACAGCCACA |
| 7815 | GCUGUAGCCGUCCUGGUUGUG | 13480 | GCTGTAGCCGTCCTGGTTGTG |
| 7816 | GCUGUCACUGUCUCUCCGGAC | 13481 | GCTGTCACTGTCTCTCCGGAC |
| 7817 | GCUGUCUAGCAAGGCUUGCAU | 13482 | GCTGTCTAGCAAGGCTTGCAT |
| 7818 | GCUGUGACACUUGAACUUGUU | 13483 | GCTGTGACACTTGAACTTGTT |
| 7819 | GCUGUGGAGCUGACCUUUAGC | 13484 | GCTGTGGAGCTGACCTTTAGC |
| 7820 | GCUGUGGAUCCAGUCCACAGC | 13485 | GCTGTGGATCCAGTCCACAGC |
| 7821 | GCUGUGUCCUAGCUGGAAACC | 13486 | GCTGTGTCCTAGCTGGAAACC |
| 7822 | GCUGUGUCCUUACGGCUGUGG | 13487 | GCTGTGTCCTTACGGCTGTGG |
| 7823 | GCUGUUGAUGUUCUUAAGCCG | 13488 | GCTGTTGATGTTCTTAAGCCG |
| 7824 | GCUGUUGCACUGGAAGCUGGC | 13489 | GCTGTTGCACTGGAAGCTGGC |
| 7825 | GCUGUUGUGUGUUGAAAGCCA | 13490 | GCTGTTGTGTGTTGAAAGCCA |
| 7826 | GCUUAAGGAAGGUACAAACCC | 13491 | GCTTAAGGAAGGTACAAACCC |
| 7827 | GCUUAGAGAUUGGUGGAUGAG | 13492 | GCTTAGAGATTGGTGGATGAG |
| 7828 | GCUUAUCACGGUUCCCUGCAC | 13493 | GCTTATCACGGTTCCCTGCAC |
| 7829 | GCUUCCCGGGUUCACACCAUU | 13494 | GCTTCCCGGGTTCACACCATT |
| 7830 | GCUUCGUUGAUGAUAUCUGUC | 13495 | GCTTCGTTGATGATATCTGTC |
| 7831 | GCUUCUCUGGGCCUGACAUCC | 13496 | GCTTCTCTGGGCCTGACATCC |
| 7832 | GCUUCUUCUCAUUUCCUCUGC | 13497 | GCTTCTTCTCATTTCCTCTGC |
| 7833 | GCUUGAGAUGGAGUGAAGUUU | 13498 | GCTTGAGATGGAGTGAAGTTT |
| 7834 | GCUUGCAUAGAAGAGGUAAAC | 13499 | GCTTGCATAGAAGAGGTAAAC |
| 7835 | GCUUGGAGCCGUUCUCCCUGA | 13500 | GCTTGGAGCCGTTCTCCCTGA |
| 7836 | GCUUGGAGGCAUUUGCAUAGA | 13501 | GCTTGGAGGCATTTGCATAGA |
| 7837 | GCUUGGUGAGACAUUGUCACU | 13502 | GCTTGGTGAGACATTGTCACT |
| 7838 | GCUUUCCCUGGCCUGGGAUCC | 13503 | GCTTTCCCTGGCCTGGGATCC |
| 7839 | GCUUUGAAGUCCCGGUCAACC | 13504 | GCTTTGAAGTCCCGGTCAACC |
| 7840 | GCUUUGGUCUUCUCUGUCUUU | 13505 | GCTTTGGTCTTCTCTGTCTTT |
| 7841 | GCUUUGUUGCUCAGGCCUGGU | 13506 | GCTTTGTTGCTCAGGCCTGGT |
| 7842 | GGAAACCCUGGCUUCCCGCGA | 13507 | GGAAACCCTGGCTTCCCGCGA |
| 7843 | GGAAACCGGGGACUUCGCCAG | 13508 | GGAAACCGGGGACTTCGCCAG |
| 7844 | GGAAAGCAGUGGCAUAAUCAU | 13509 | GGAAAGCAGTGGCATAATCAT |
| 7845 | GGAAAGGAAGAAACCAAAAUC | 13510 | GGAAAGGAAGAAACCAAAATC |
| 7846 | GGAACACGUAAAGACCCCUAC | 13511 | GGAACACGTAAAGACCCCTAC |
| 7847 | GGAACAGCCGCCGUUGUUGUC | 13512 | GGAACAGCCGCCGTTGTTGTC |
| 7848 | GGAACAGGUCGGGUGGUUGUG | 13513 | GGAACAGGTCGGGTGGTTGTG |
| 7849 | GGAACCGAGGCCUUGAGGGUG | 13514 | GGAACCGAGGCCTTGAGGGTG |
| 7850 | GGAACCGCGGAGGAACGCCAG | 13515 | GGAACCGCGGAGGAACGCCAG |
| 7851 | GGAACCUGUGAGGCGGUUGGC | 13516 | GGAACCTGTGAGGCGGTTGGC |
| 7852 | GGAACUCGAAGGCCGAGCAGG | 13517 | GGAACTCGAAGGCCGAGCAGG |
| 7853 | GGAACUCGUUUCUUUCGCAUC | 13518 | GGAACTCGTTTCTTTCGCATC |
| 7854 | GGAAGAAACCAAAAUCCCAAC | 13519 | GGAAGAAACCAAAATCCCAAC |
| 7855 | GGAAGACCCCCAGGCAAAGGA | 13520 | GGAAGACCCCCAGGCAAAGGA |
| 7856 | GGAAGACGAGGAGCACGAUGG | 13521 | GGAAGACGAGGAGCACGATGG |
| 7857 | GGAAGACGAGGAGCACUGUAG | 13522 | GGAAGACGAGGAGCACTGTAG |
| 7858 | GGAAGAGAACCAUAUCCUCUG | 13523 | GGAAGAGAACCATATCCTCTG |
| 7859 | GGAAGAGACGCCGUGGGCUCU | 13524 | GGAAGAGACGCCGTGGGCTCT |
| 7860 | GGAAGCACUAGGUGGGCGGAC | 13525 | GGAAGCACTAGGTGGGCGGAC |
| 7861 | GGAAGCCGUCGGGGCACAGGC | 13526 | GGAAGCCGTCGGGGCACAGGC |
| 7862 | GGAAGCCUUCCUCACACUGGC | 13527 | GGAAGCCTTCCTCACACTGGC |
| 7863 | GGAAGCUGGCGGGACCACAGG | 13528 | GGAAGCTGGCGGGACCACAGG |
| 7864 | GGAAGGGUUCUGGGCAGGGAC | 13529 | GGAAGGGTTCTGGGCAGGGAC |
| 7865 | GGAAGGUACAAACCCACCUCG | 13530 | GGAAGGTACAAACCCACCTCG |
| 7866 | GGAAGUGAAUGGCUUGGAGGC | 13531 | GGAAGTGAATGGCTTGGAGGC |
| 7867 | GGAAGUGGCAUCAUUUGGUGA | 13532 | GGAAGTGGCATCATTTGGTGA |
| 7868 | GGAAGUGGGUAGGGGUCGGGA | 13533 | GGAAGTGGGTAGGGGTCGGGA |
| 7869 | GGAAUGCAGCGGUUGACACGG | 13534 | GGAATGCAGCGGTTGACACGG |
| 7870 | GGAAUGGAAGUGGGUAGGGGU | 13535 | GGAATGGAAGTGGGTAGGGGT |
| 7871 | GGAAUUCGUCAGGGCGACAGG | 13536 | GGAATTCGTCAGGGCGACAGG |
| 7872 | GGACAAGGUGGGGACGGAGGU | 13537 | GGACAAGGTGGGGACGGAGGT |
| 7873 | GGACAAUGGACAGAGCCCUCA | 13538 | GGACAATGGACAGAGCCCTCA |
| 7874 | GGACAAUUGCCAAUCCCUUGU | 13539 | GGACAATTGCCAATCCCTTGT |
| 7875 | GGACACAGUGUUUUGUCCCUG | 13540 | GGACACAGTGTTTTGTCCCTG |
| 7876 | GGACAGAGCCCUCACGCUACU | 13541 | GGACAGAGCCCTCACGCTACT |
| 7877 | GGACAGAGUCGGUCCAGUAGA | 13542 | GGACAGAGTCGGTCCAGTAGA |
| 7878 | GGACAGCCUUGCUCGUCUGAG | 13543 | GGACAGCCTTGCTCGTCTGAG |
| 7879 | GGACAGGUCAGACCAGUAGAU | 13544 | GGACAGGTCAGACCAGTAGAT |
| 7880 | GGACAGUAGGUUUUCAGCCAA | 13545 | GGACAGTAGGTTTTCAGCCAA |
| 7881 | GGACAUAAAGACGACAAUGGC | 13546 | GGACATAAAGACGACAATGGC |
| 7882 | GGACAUCAGUGCAACAGUAAC | 13547 | GGACATCAGTGCAACAGTAAC |
| 7883 | GGACAUCAUUCUCCCAAAAAG | 13548 | GGACATCATTCTCCCAAAAAG |
| 7884 | GGACCACAGGUGAGCACCGGG | 13549 | GGACCACAGGTGAGCACCGGG |
| 7885 | GGACCCGGGACUUGUCGUCGC | 13550 | GGACCCGGGACTTGTCGTCGC |
| 7886 | GGACCUCAUCCUCUGUGGUCU | 13551 | GGACCTCATCCTCTGTGGTCT |
| 7887 | GGACGCAUUUACGUGCUCCGA | 13552 | GGACGCATTTACGTGCTCCGA |
| 7888 | GGACGGAGGUGUCGCCACAGA | 13553 | GGACGGAGGTGTCGCCACAGA |
| 7889 | GGACGGGACUCCAGGCAGAUG | 13554 | GGACGGGACTCCAGGCAGATG |
| 7890 | GGACUACAGGUGCCCGCCACC | 13555 | GGACTACAGGTGCCCGCCACC |
| 7891 | GGACUCAUCAGAGCCAUCCUG | 13556 | GGACTCATCAGAGCCATCCTG |
| 7892 | GGACUCCAGGCAGAUGUUCAC | 13557 | GGACTCCAGGCAGATGTTCAC |
| 7893 | GGACUGCAGUGGCGCAAUCUC | 13558 | GGACTGCAGTGGCGCAATCTC |
| 7894 | GGACUUCGCCAGGAGGGCCCA | 13559 | GGACTTCGCCAGGAGGGCCCA |
| 7895 | GGACUUGUCGUCGCCUUUGUG | 13560 | GGACTTGTCGTCGCCTTTGTG |
| 7896 | GGAGAAGGGGUGGGCCAGCCU | 13561 | GGAGAAGGGGTGGGCCAGCCT |
| 7897 | GGAGACCACGGGAAUGGAAGU | 13562 | GGAGACCACGGGAATGGAAGT |
| 7898 | GGAGAGAAACUCAAAACUUCC | 13563 | GGAGAGAAACTCAAAACTTCC |
| 7899 | GGAGAGAAAUGGAGGUGUCAU | 13564 | GGAGAGAAATGGAGGTGTCAT |
| 7900 | GGAGAGGCACGCUCUGUCACA | 13565 | GGAGAGGCACGCTCTGTCACA |
| 7901 | GGAGAUCUAGGGUGAUGCCAU | 13566 | GGAGATCTAGGGTGATGCCAT |
| 7902 | GGAGAUGCAUUUCCCGUCUUG | 13567 | GGAGATGCATTTCCCGTCTTG |
| 7903 | GGAGAUUCUGAGGCAACGUUU | 13568 | GGAGATTCTGAGGCAACGTTT |
| 7904 | GGAGCAAGGCGACGGUCCAGC | 13569 | GGAGCAAGGCGACGGTCCAGC |
| 7905 | GGAGCACGAUGGGGAGGACAA | 13570 | GGAGCACGATGGGGAGGACAA |
| 7906 | GGAGCACGUCUCCUGGGACUC | 13571 | GGAGCACGTCTCCTGGGACTC |
| 7907 | GGAGCACGUCUUGGGGGAGCA | 13572 | GGAGCACGTCTTGGGGGAGCA |
| 7908 | GGAGCACGUCUUGGGGGGACA | 13573 | GGAGCACGTCTTGGGGGGACA |
| 7909 | GGAGCACUGUAGGGAGAGGCA | 13574 | GGAGCACTGTAGGGAGAGGCA |
| 7910 | GGAGCCCACAGCCUUGCAGGC | 13575 | GGAGCCCACAGCCTTGCAGGC |
| 7911 | GGAGCCGUUCUCCCUGAAUAA | 13576 | GGAGCCGTTCTCCCTGAATAA |
| 7912 | GGAGCUGACCUUUAGCCUGAC | 13577 | GGAGCTGACCTTTAGCCTGAC |
| 7913 | GGAGCUGUUGCACUGGAAGCU | 13578 | GGAGCTGTTGCACTGGAAGCT |
| 7914 | GGAGGAACGCCAGGCCUCCCC | 13579 | GGAGGAACGCCAGGCCTCCCC |
| 7915 | GGAGGACAAUGGACAGAGCCC | 13580 | GGAGGACAATGGACAGAGCCC |
| 7916 | GGAGGAUGGGCACCAGGCUGG | 13581 | GGAGGATGGGCACCAGGCTGG |
| 7917 | GGAGGAUUUAUUUAUUUUUUU | 13582 | GGAGGATTTATTTATTTTTTT |
| 7918 | GGAGGCACAGAUACUGGCAGC | 13583 | GGAGGCACAGATACTGGCAGC |
| 7919 | GGAGGCAUUUGCAUAGAAGUG | 13584 | GGAGGCATTTGCATAGAAGTG |
| 7920 | GGAGGCCUCGUCUGAGCCGUC | 13585 | GGAGGCCTCGTCTGAGCCGTC |
| 7921 | GGAGGCGGCUCCGGGGACCCG | 13586 | GGAGGCGGCTCCGGGGACCCG |
| 7922 | GGAGGCUGUCACUGUCUCUCC | 13587 | GGAGGCTGTCACTGTCTCTCC |
| 7923 | GGAGGGCCCAGGAUAACGGAG | 13588 | GGAGGGCCCAGGATAACGGAG |
| 7924 | GGAGGGCGACUCGUGUAUGUC | 13589 | GGAGGGCGACTCGTGTATGTC |
| 7925 | GGAGGGGCGGGGAGAUUCUGA | 13590 | GGAGGGGGGGGGAGATTCTGA |
| 7926 | GGAGGUGUCAUCCUGGUCCCC | 13591 | GGAGGTGTCATCCTGGTCCCC |
| 7927 | GGAGGUGUCGCCACAGAGCAC | 13592 | GGAGGTGTCGCCACAGAGCAC |
| 7928 | GGAGGUGUCGGGAACAGGUCG | 13593 | GGAGGTGTCGGGAACAGGTCG |
| 7929 | GGAGUCAACCCAGUAGAGGCG | 13594 | GGAGTCAACCCAGTAGAGGCG |
| 7930 | GGAGUCUCGCUCUGUCGCCCA | 13595 | GGAGTCTCGCTCTGTCGCCCA |
| 7931 | GGAGUCUGACGGAGGCUGUCA | 13596 | GGAGTCTGACGGAGGCTGTCA |
| 7932 | GGAGUGAAGUUUGGAGUCAAC | 13597 | GGAGTGAAGTTTGGAGTCAAC |
| 7933 | GGAGUGCAGUGGCUCGAUCAU | 13598 | GGAGTGCAGTGGCTCGATCAT |
| 7934 | GGAGUGGAUGCACUCGCCACU | 13599 | GGAGTGGATGCACTCGCCACT |
| 7935 | GGAGUUCCCCAGUCAGUCCAG | 13600 | GGAGTTCCCCAGTCAGTCCAG |
| 7936 | GGAUAACGGAGGGCGACUCGU | 13601 | GGATAACGGAGGGCGACTCGT |
| 7937 | GGAUCACAGGCACGUGCCACC | 13602 | GGATCACAGGCACGTGCCACC |
| 7938 | GGAUCACGACCUGCUGUGUCC | 13603 | GGATCACGACCTGCTGTGTCC |
| 7939 | GGAUCCACCACGAUGGCCCUU | 13604 | GGATCCACCACGATGGCCCTT |
| 7940 | GGAUCCAGUCCACAGCCAGCC | 13605 | GGATCCAGTCCACAGCCAGCC |
| 7941 | GGAUCCCAUCCCAACACACAC | 13606 | GGATCCCATCCCAACACACAC |
| 7942 | GGAUCCUGACACUCAUCGAUA | 13607 | GGATCCTGACACTCATCGATA |
| 7943 | GGAUCUCGCUUUGUUGCUCAG | 13608 | GGATCTCGCTTTGTTGCTCAG |
| 7944 | GGAUGAGGCAGGGCCAGGGGG | 13609 | GGATGAGGCAGGGCCAGGGGG |
| 7945 | GGAUGAGGCUGGUGUACUCGC | 13610 | GGATGAGGCTGGTGTACTCGC |
| 7946 | GGAUGCACUCGCCACUUAGGC | 13611 | GGATGCACTCGCCACTTAGGC |
| 7947 | GGAUGCAGGUGGAGCUGUUGC | 13612 | GGATGCAGGTGGAGCTGTTGC |
| 7948 | GGAUGCAGUUUCCAUCAGAGC | 13613 | GGATGCAGTTTCCATCAGAGC |
| 7949 | GGAUGGGCACCAGGCUGGUGU | 13614 | GGATGGGCACCAGGCTGGTGT |
| 7950 | GGAUGGUCUCGAUCUCCUGAC | 13615 | GGATGGTCTCGATCTCCTGAC |
| 7951 | GGAUGUCUCCUGGGUGGCCAC | 13616 | GGATGTCTCCTGGGTGGCCAC |
| 7952 | GGAUGUCUCUGCUGAUGACGG | 13617 | GGATGTCTCTGCTGATGACGG |
| 7953 | GGAUUACAGGCGUGAGCCACU | 13618 | GGATTACAGGCGTGAGCCACT |
| 7954 | GGAUUACAGGCGUGAGGCACC | 13619 | GGATTACAGGCGTGAGGCACC |
| 7955 | GGAUUCAGCCAGAUCAUUUCC | 13620 | GGATTCAGCCAGATCATTTCC |
| 7956 | GGAUUGUGUCACGGUGGAGAG | 13621 | GGATTGTGTCACGGTGGAGAG |
| 7957 | GGAUUUAUUUAUUUUUUUAUA | 13622 | GGATTTATTTATTTTTTTATA |
| 7958 | GGAUUUGCAGGUGACAGACAA | 13623 | GGATTTGCAGGTGACAGACAA |
| 7959 | GGCAAAGGAAGACGAGGAGCA | 13624 | GGCAAAGGAAGACGAGGAGCA |
| 7960 | GGCAAAGGCUAACCUGGCUGU | 13625 | GGCAAAGGCTAACCTGGCTGT |
| 7961 | GGCAAAUGUGGACCUCAUCCU | 13626 | GGCAAATGTGGACCTCATCCT |
| 7962 | GGCAACGUUUCAACCUCGUAA | 13627 | GGCAACGTTTCAACCTCGTAA |
| 7963 | GGCAAUAACCCCCUACACAGG | 13628 | GGCAATAACCCCCTACACAGG |
| 7964 | GGCAAUGCUUUGGUCUUCUCU | 13629 | GGCAATGCTTTGGTCTTCTCT |
| 7965 | GGCACAGAUACUGGCAGCCGC | 13630 | GGCACAGATACTGGCAGCCGC |
| 7966 | GGCACAGGCACUCGUAGCCGA | 13631 | GGCACAGGCACTCGTAGCCGA |
| 7967 | GGCACCAGGCUGGUGUCCUGA | 13632 | GGCACCAGGCTGGTGTCCTGA |
| 7968 | GGCACCUGUGUCUGGCCAAAU | 13633 | GGCACCTGTGTCTGGCCAAAT |
| 7969 | GGCACGCUCUGUCACACAGCU | 13634 | GGCACGCTCTGTCACACAGCT |
| 7970 | GGCACGUGCCACCGUACCCAG | 13635 | GGCACGTGCCACCGTACCCAG |
| 7971 | GGCACUCAGCGCUGCCAUCGC | 13636 | GGCACTCAGCGCTGCCATCGC |
| 7972 | GGCACUCGUAGCCGAUCUUAA | 13637 | GGCACTCGTAGCCGATCTTAA |
| 7973 | GGCACUGAAAAUGGCUUCGUU | 13638 | GGCACTGAAAATGGCTTCGTT |
| 7974 | GGCACUGGAACUCGUUUCUUU | 13639 | GGCACTGGAACTCGTTTCTTT |
| 7975 | GGCACUGUCCGAAGCCUGUUC | 13640 | GGCACTGTCCGAAGCCTGTTC |
| 7976 | GGCACUUAAUAAAUAUUAAGG | 13641 | GGCACTTAATAAATATTAAGG |
| 7977 | GGCACUUGUAGCCACCCUCCA | 13642 | GGCACTTGTAGCCACCCTCCA |
| 7978 | GGCAGAUGUUCACGCCACGUC | 13643 | GGCAGATGTTCACGCCACGTC |
| 7979 | GGCAGCCGCCAUUGCUCAGGG | 13644 | GGCAGCCGCCATTGCTCAGGG |
| 7980 | GGCAGCCGGGAGGUGUCGGGA | 13645 | GGCAGCCGGGAGGTGTCGGGA |
| 7981 | GGCAGCGAAACUCGUCCUGGG | 13646 | GGCAGCGAAACTCGTCCTGGG |
| 7982 | GGCAGCUCCUCAUGUCCCUGG | 13647 | GGCAGCTCCTCATGTCCCTGG |
| 7983 | GGCAGGAGGCCUCGUCUGAGC | 13648 | GGCAGGAGGCCTCGTCTGAGC |
| 7984 | GGCAGGCAAUGCUUUGGUCUU | 13649 | GGCAGGCAATGCTTTGGTCTT |
| 7985 | GGCAGGCGCAGGUAAACUUGG | 13650 | GGCAGGCGCAGGTAAACTTGG |
| 7986 | GGCAGGGACGGGACUCCAGGC | 13651 | GGCAGGGACGGGACTCCAGGC |
| 7987 | GGCAGGGAGGCACAGAUACUG | 13652 | GGCAGGGAGGCACAGATACTG |
| 7988 | GGCAGGGCCAGGGGGUCCCGU | 13653 | GGCAGGGCCAGGGGGTCCCGT |
| 7989 | GGCAGUCUCUAGCCAUGUUGC | 13654 | GGCAGTCTCTAGCCATGTTGC |
| 7990 | GGCAGUGGAACUCGAAGGCCG | 13655 | GGCAGTGGAACTCGAAGGCCG |
| 7991 | GGCAGUGUCCCGACCCGGAUC | 13656 | GGCAGTGTCCCGACCCGGATC |
| 7992 | GGCAGUUCUCGAUAUUCACGA | 13657 | GGCAGTTCTCGATATTCACGA |
| 7993 | GGCAUAAUCAUAGCUCACGAC | 13658 | GGCATAATCATAGCTCACGAC |
| 7994 | GGCAUCAUUUGGUGAAUGAGU | 13659 | GGCATCATTTGGTGAATGAGT |
| 7995 | GGCAUUCAUUGACACGGGCUU | 13660 | GGCATTCATTGACACGGGCTT |
| 7996 | GGCAUUUCAGAGGCAAUAACC | 13661 | GGCATTTCAGAGGCAATAACC |
| 7997 | GGCAUUUGCAUAGAAGUGGAA | 13662 | GGCATTTGCATAGAAGTGGAA |
| 7998 | GGCCAAAUUCAUUUUUUAAGA | 13663 | GGCCAAATTCATTTTTTAAGA |
| 7999 | GGCCAAGGAGAAGGGGUGGGC | 13664 | GGCCAAGGAGAAGGGGTGGGC |
| 8000 | GGCCAAGGUAACCGGGUGUCU | 13665 | GGCCAAGGTAACCGGGTGTCT |
| 8001 | GGCCACAGCGCAGUUUUCCUC | 13666 | GGCCACAGCGCAGTTTTCCTC |
| 8002 | GGCCACAGGACAGCCUUGCUC | 13667 | GGCCACAGGACAGCCTTGCTC |
| 8003 | GGCCACCAGCUGGAAGCCGUC | 13668 | GGCCACCAGCTGGAAGCCGTC |
| 8004 | GGCCACCAUCACAGCGCCAGC | 13669 | GGCCACCATCACAGCGCCAGC |
| 8005 | GGCCACCUCCGUGUCCAGAGC | 13670 | GGCCACCTCCGTGTCCAGAGC |
| 8006 | GGCCACGUGUCCUCACGGCCA | 13671 | GGCCACGTGTCCTCACGGCCA |
| 8007 | GGCCACUCAUCCGAGCCAUCU | 13672 | GGCCACTCATCCGAGCCATCT |
| 8008 | GGCCACUGAAUGUUUUCAGUC | 13673 | GGCCACTGAATGTTTTCAGTC |
| 8009 | GGCCACUGAGGAGAUCUAGGG | 13674 | GGCCACTGAGGAGATCTAGGG |
| 8010 | GGCCACUGCAGCCUCAGCCUC | 13675 | GGCCACTGCAGCCTCAGCCTC |
| 8011 | GGCCAGACUGCGGACUGCAGU | 13676 | GGCCAGACTGCGGACTGCAGT |
| 8012 | GGCCAGAGGCCAUGGGCUGCU | 13677 | GGCCAGAGGCCATGGGCTGCT |
| 8013 | GGCCAGCAGCAUGCCGUCCGG | 13678 | GGCCAGCAGCATGCCGTCCGG |
| 8014 | GGCCAGCCUCUUUUCAUCCUC | 13679 | GGCCAGCCTCTTTTCATCCTC |
| 8015 | GGCCAGGGGGUCCCGUUGGUG | 13680 | GGCCAGGGGGTCCCGTTGGTG |
| 8016 | GGCCAUCGCACCUCCAGAACU | 13681 | GGCCATCGCACCTCCAGAACT |
| 8017 | GGCCAUGGGCUGCUGAUGCAC | 13682 | GGCCATGGGCTGCTGATGCAC |
| 8018 | GGCCCACAGCUGGGGGAUGCA | 13683 | GGCCCACAGCTGGGGGATGCA |
| 8019 | GGCCCAGGAUAACGGAGGGCG | 13684 | GGCCCAGGATAACGGAGGGCG |
| 8020 | GGCCCCAGGCAGCCGGGAGGU | 13685 | GGCCCCAGGCAGCCGGGAGGT |
| 8021 | GGCCCCAUGCUCGCAGCCUCU | 13686 | GGCCCCATGCTCGCAGCCTCT |
| 8022 | GGCCCCCACAGCUGAAGUCCC | 13687 | GGCCCCCACAGCTGAAGTCCC |
| 8023 | GGCCCCCUUUCUUGAUCUUGG | 13688 | GGCCCCCTTTCTTGATCTTGG |
| 8024 | GGCCCCGAGAAUAGUCACUGA | 13689 | GGCCCCGAGAATAGTCACTGA |
| 8025 | GGCCCCUCUGUGAACUUGAUC | 13690 | GGCCCCTCTGTGAACTTGATC |
| 8026 | GGCCCUUGGCUUGGAGCCGUU | 13691 | GGCCCTTGGCTTGGAGCCGTT |
| 8027 | GGCCGAGCAGGGGCUACUGUC | 13692 | GGCCGAGCAGGGGCTACTGTC |
| 8028 | GGCCGGCGAGGUCUCAGGAAG | 13693 | GGCCGGCGAGGTCTCAGGAAG |
| 8029 | GGCCGGGGGCCCCUCUGUGAA | 13694 | GGCCGGGGGCCCCTCTGTGAA |
| 8030 | GGCCUCACCGUGCAUGUUUUA | 13695 | GGCCTCACCGTGCATGTTTTA |
| 8031 | GGCCUCCCAAAGUGCUGGGAU | 13696 | GGCCTCCCAAAGTGCTGGGAT |
| 8032 | GGCCUCGUCUGAGCCGUCCAA | 13697 | GGCCTCGTCTGAGCCGTCCAA |
| 8033 | GGCCUGACAUCCCCUCCCCUU | 13698 | GGCCTGACATCCCCTCCCCTT |
| 8034 | GGCCUGGAUGUCUCUGCUGAU | 13699 | GGCCTGGATGTCTCTGCTGAT |
| 8035 | GGCCUGGGAUCCCAUCCCAAC | 13700 | GGCCTGGGATCCCATCCCAAC |
| 8036 | GGCCUGGUCUCCAACUCCUGA | 13701 | GGCCTGGTCTCCAACTCCTGA |
| 8037 | GGCCUGUUUCUCUUAUCCUUC | 13702 | GGCCTGTTTCTCTTATCCTTC |
| 8038 | GGCCUUCGUGUGGGGGUCCAG | 13703 | GGCCTTCGTGTGGGGGTCCAG |
| 8039 | GGCCUUGAGGGUGGCCGGGGG | 13704 | GGCCTTGAGGGTGGCCGGGGG |
| 8040 | GGCGACAGGUGGCCACAGCGC | 13705 | GGCGACAGGTGGCCACAGCGC |
| 8041 | GGCGACAGGUGGCCACAGGAC | 13706 | GGCGACAGGTGGCCACAGGAC |
| 8042 | GGCGACGGUCCAGCGCAAUUU | 13707 | GGCGACGGTCCAGCGCAATTT |
| 8043 | GGCGACUCGUGUAUGUCGCGG | 13708 | GGCGACTCGTGTATGTCGCGG |
| 8044 | GGCGAGGAGCAAGGCGACGGU | 13709 | GGCGAGGAGCAAGGCGACGGT |
| 8045 | GGCGAGGGCGCCCCCCAGGAC | 13710 | GGCGAGGGCGCCCCCCAGGAC |
| 8046 | GGCGAGGUCUCAGGAAGGGUU | 13711 | GGCGAGGTCTCAGGAAGGGTT |
| 8047 | GGCGAGUGGGGGUUGAUCUGC | 13712 | GGCGAGTGGGGGTTGATCTGC |
| 8048 | GGCGAUCCUCCUGCCUCAGCC | 13713 | GGCGATCCTCCTGCCTCAGCC |
| 8049 | GGCGAUGGAGCCCACAGCCUU | 13714 | GGCGATGGAGCCCACAGCCTT |
| 8050 | GGCGAUGGGGAGGACAAUGGA | 13715 | GGCGATGGGGAGGACAATGGA |
| 8051 | GGCGAUUUCAGAAAGAACCUG | 13716 | GGCGATTTCAGAAAGAACCTG |
| 8052 | GGCGCAAUCUCGGCUCACUGC | 13717 | GGCGCAATCTCGGCTCACTGC |
| 8053 | GGCGCAGGUAAACUUGGGCGA | 13718 | GGCGCAGGTAAACTTGGGCGA |
| 8054 | GGCGCCCCCCAGGACAAGGUG | 13719 | GGCGCCCCCCAGGACAAGGTG |
| 8055 | GGCGGACAUAAAGACGACAAU | 13720 | GGCGGACATAAAGACGACAAT |
| 8056 | GGCGGCCACUGAGGAGAUCUA | 13721 | GGCGGCCACTGAGGAGATCTA |
| 8057 | GGCGGCUCCGGGGACCCGGGA | 13722 | GGCGGCTCCGGGGACCCGGGA |
| 8058 | GGCGGGACCACAGGUGAGCAC | 13723 | GGCGGGACCACAGGTGAGCAC |
| 8059 | GGCGGGAGUUCCCCAGUCAGU | 13724 | GGCGGGAGTTCCCCAGTCAGT |
| 8060 | GGCGGGGAGAUUCUGAGGCAA | 13725 | GGCGGGGAGATTCTGAGGCAA |
| 8061 | GGCGGUUGGCACUGAAAAUGG | 13726 | GGCGGTTGGCACTGAAAATGG |
| 8062 | GGCGUGAGCCACUGCGCCCGG | 13727 | GGCGTGAGCCACTGCGCCCGG |
| 8063 | GGCGUGAGGCACCUGUGUCUG | 13728 | GGCGTGAGGCACCTGTGTCTG |
| 8064 | GGCUAACCUGGCUGUCUAGCA | 13729 | GGCTAACCTGGCTGTCTAGCA |
| 8065 | GGCUAAUUUUUUGUAUUUUUA | 13730 | GGCTAATTTTTTGTATTTTTA |
| 8066 | GGCUACAAAAACCUUCUAUAC | 13731 | GGCTACAAAAACCTTCTATAC |
| 8067 | GGCUACUGUCCCCUUGGAACA | 13732 | GGCTACTGTCCCCTTGGAACA |
| 8068 | GGCUCACUGCAACCUCCACCU | 13733 | GGCTCACTGCAACCTCCACCT |
| 8069 | GGCUCACUGCAAGCUCCGCUU | 13734 | GGCTCACTGCAAGCTCCGCTT |
| 8070 | GGCUCAGGCGAUCCUCCUGCC | 13735 | GGCTCAGGCGATCCTCCTGCC |
| 8071 | GGCUCCGGGGACCCGGGACUU | 13736 | GGCTCCGGGGACCCGGGACTT |
| 8072 | GGCUCGAUCAUGGCUCACUGC | 13737 | GGCTCGATCATGGCTCACTGC |
| 8073 | GGCUCUGCCUCUGGGAAGUGG | 13738 | GGCTCTGCCTCTGGGAAGTGG |
| 8074 | GGCUCUGUCAAGCUGGGUGCU | 13739 | GGCTCTGTCAAGCTGGGTGCT |
| 8075 | GGCUGAAGAUCUCGGCUGCAG | 13740 | GGCTGAAGATCTCGGCTGCAG |
| 8076 | GGCUGCAGACCAAGGAGGGGC | 13741 | GGCTGCAGACCAAGGAGGGGC |
| 8077 | GGCUGCAGGUGUCGGGAUCCU | 13742 | GGCTGCAGGTGTCGGGATCCT |
| 8078 | GGCUGCCAUGGAUGCAGUUUC | 13743 | GGCTGCCATGGATGCAGTTTC |
| 8079 | GGCUGCUGAUGCACCAUGCAU | 13744 | GGCTGCTGATGCACCATGCAT |
| 8080 | GGCUGGAAAGCAGUGGCAUAA | 13745 | GGCTGGAAAGCAGTGGCATAA |
| 8081 | GGCUGGAGUGCAGUGGCUCGA | 13746 | GGCTGGAGTGCAGTGGCTCGA |
| 8082 | GGCUGGGUGAGGUUGUGGAAG | 13747 | GGCTGGGTGAGGTTGTGGAAG |
| 8083 | GGCUGGUGGGGCGAGGGCGCC | 13748 | GGCTGGTGGGGCGAGGGCGCC |
| 8084 | GGCUGGUGUACUCGCUCCGGU | 13749 | GGCTGGTGTACTCGCTCCGGT |
| 8085 | GGCUGGUGUCCUGACAGCCAC | 13750 | GGCTGGTGTCCTGACAGCCAC |
| 8086 | GGCUGUCACUGUCUCUCCGGA | 13751 | GGCTGTCACTGTCTCTCCGGA |
| 8087 | GGCUGUCUAGCAAGGCUUGCA | 13752 | GGCTGTCTAGCAAGGCTTGCA |
| 8088 | GGCUGUGGAGCUGACCUUUAG | 13753 | GGCTGTGGAGCTGACCTTTAG |
| 8089 | GGCUUAAGGAAGGUACAAACC | 13754 | GGCTTAAGGAAGGTACAAACC |
| 8090 | GGCUUAGAGAUUGGUGGAUGA | 13755 | GGCTTAGAGATTGGTGGATGA |
| 8091 | GGCUUAUCACGGUUCCCUGCA | 13756 | GGCTTATCACGGTTCCCTGCA |
| 8092 | GGCUUCGUUGAUGAUAUCUGU | 13757 | GGCTTCGTTGATGATATCTGT |
| 8093 | GGCUUCUUCUCAUUUCCUCUG | 13758 | GGCTTCTTCTCATTTCCTCTG |
| 8094 | GGCUUGCAUAGAAGAGGUAAA | 13759 | GGCTTGCATAGAAGAGGTAAA |
| 8095 | GGCUUGGAGCCGUUCUCCCUG | 13760 | GGCTTGGAGCCGTTCTCCCTG |
| 8096 | GGCUUGGAGGCAUUUGCAUAG | 13761 | GGCTTGGAGGCATTTGCATAG |
| 8097 | GGCUUUCCCUGGCCUGGGAUC | 13762 | GGCTTTCCCTGGCCTGGGATC |
| 8098 | GGCUUUGAAGUCCCGGUCAAC | 13763 | GGCTTTGAAGTCCCGGTCAAC |
| 8099 | GGGAACAGCCGCCGUUGUUGU | 13764 | GGGAACAGCCGCCGTTGTTGT |
| 8100 | GGGAACAGGUCGGGUGGUUGU | 13765 | GGGAACAGGTCGGGTGGTTGT |
| 8101 | GGGAAGUGAAUGGCUUGGAGG | 13766 | GGGAAGTGAATGGCTTGGAGG |
| 8102 | GGGAAGUGGCAUCAUUUGGUG | 13767 | GGGAAGTGGCATCATTTGGTG |
| 8103 | GGGAAUGGAAGUGGGUAGGGG | 13768 | GGGAATGGAAGTGGGTAGGGG |
| 8104 | GGGACAAUUGCCAAUCCCUUG | 13769 | GGGACAATTGCCAATCCCTTG |
| 8105 | GGGACACAGUGUUUUGUCCCU | 13770 | GGGACACAGTGTTTTGTCCCT |
| 8106 | GGGACAGCCUUGCUCGUCUGA | 13771 | GGGACAGCCTTGCTCGTCTGA |
| 8107 | GGGACAGGUCAGACCAGUAGA | 13772 | GGGACAGGTCAGACCAGTAGA |
| 8108 | GGGACAGUAGGUUUUCAGCCA | 13773 | GGGACAGTAGGTTTTCAGCCA |
| 8109 | GGGACAUCAUUCUCCCAAAAA | 13774 | GGGACATCATTCTCCCAAAAA |
| 8110 | GGGACCACAGGUGAGCACCGG | 13775 | GGGACCACAGGTGAGCACCGG |
| 8111 | GGGACCCGGGACUUGUCGUCG | 13776 | GGGACCCGGGACTTGTCGTCG |
| 8112 | GGGACGCAUUUACGUGCUCCG | 13777 | GGGACGCATTTACGTGCTCCG |
| 8113 | GGGACGGAGGUGUCGCCACAG | 13778 | GGGACGGAGGTGTCGCCACAG |
| 8114 | GGGACGGGACUCCAGGCAGAU | 13779 | GGGACGGGACTCCAGGCAGAT |
| 8115 | GGGACUACAGGUGCCCGCCAC | 13780 | GGGACTACAGGTGCCCGCCAC |
| 8116 | GGGACUCAUCAGAGCCAUCCU | 13781 | GGGACTCATCAGAGCCATCCT |
| 8117 | GGGACUCCAGGCAGAUGUUCA | 13782 | GGGACTCCAGGCAGATGTTCA |
| 8118 | GGGACUUCGCCAGGAGGGCCC | 13783 | GGGACTTCGCCAGGAGGGCCC |
| 8119 | GGGACUUGUCGUCGCCUUUGU | 13784 | GGGACTTGTCGTCGCCTTTGT |
| 8120 | GGGAGAGGCACGCUCUGUCAC | 13785 | GGGAGAGGCACGCTCTGTCAC |
| 8121 | GGGAGAUUCUGAGGCAACGUU | 13786 | GGGAGATTCTGAGGCAACGTT |
| 8122 | GGGAGCACGUCUCCUGGGACU | 13787 | GGGAGCACGTCTCCTGGGACT |
| 8123 | GGGAGCACGUCUUGGGGGAGC | 13788 | GGGAGCACGTCTTGGGGGAGC |
| 8124 | GGGAGCACGUCUUGGGGGGAC | 13789 | GGGAGCACGTCTTGGGGGGAC |
| 8125 | GGGAGGACAAUGGACAGAGCC | 13790 | GGGAGGACAATGGACAGAGCC |
| 8126 | GGGAGGAUGGGCACCAGGCUG | 13791 | GGGAGGATGGGCACCAGGCTG |
| 8127 | GGGAGGCACAGAUACUGGCAG | 13792 | GGGAGGCACAGATACTGGCAG |
| 8128 | GGGAGGUGUCGGGAACAGGUC | 13793 | GGGAGGTGTCGGGAACAGGTC |
| 8129 | GGGAGUCUGACGGAGGCUGUC | 13794 | GGGAGTCTGACGGAGGCTGTC |
| 8130 | GGGAGUUCCCCAGUCAGUCCA | 13795 | GGGAGTTCCCCAGTCAGTCCA |
| 8131 | GGGAUCACAGGCACGUGCCAC | 13796 | GGGATCACAGGCACGTGCCAC |
| 8132 | GGGAUCCCAUCCCAACACACA | 13797 | GGGATCCCATCCCAACACACA |
| 8133 | GGGAUCCUGACACUCAUCGAU | 13798 | GGGATCCTGACACTCATCGAT |
| 8134 | GGGAUCUCGCUUUGUUGCUCA | 13799 | GGGATCTCGCTTTGTTGCTCA |
| 8135 | GGGAUGAGGCUGGUGUACUCG | 13800 | GGGATGAGGCTGGTGTACTCG |
| 8136 | GGGAUGCAGGUGGAGCUGUUG | 13801 | GGGATGCAGGTGGAGCTGTTG |
| 8137 | GGGAUUACAGGCGUGAGCCAC | 13802 | GGGATTACAGGCGTGAGCCAC |
| 8138 | GGGAUUACAGGCGUGAGGCAC | 13803 | GGGATTACAGGCGTGAGGCAC |
| 8139 | GGGCAAAGGCUAACCUGGCUG | 13804 | GGGCAAAGGCTAACCTGGCTG |
| 8140 | GGGCACAGGCACUCGUAGCCG | 13805 | GGGCACAGGCACTCGTAGCCG |
| 8141 | GGGCACCAGGCUGGUGUCCUG | 13806 | GGGCACCAGGCTGGTGTCCTG |
| 8142 | GGGCACUGUCCGAAGCCUGUU | 13807 | GGGCACTGTCCGAAGCCTGTT |
| 8143 | GGGCAGGAGGCCUCGUCUGAG | 13808 | GGGCAGGAGGCCTCGTCTGAG |
| 8144 | GGGCAGGCGCAGGUAAACUUG | 13809 | GGGCAGGCGCAGGTAAACTTG |
| 8145 | GGGCAGGGACGGGACUCCAGG | 13810 | GGGCAGGGACGGGACTCCAGG |
| 8146 | GGGCAGGGAGGCACAGAUACU | 13811 | GGGCAGGGAGGCACAGATACT |
| 8147 | GGGCCACCAGCUGGAAGCCGU | 13812 | GGGCCACCAGCTGGAAGCCGT |
| 8148 | GGGCCACCAUCACAGCGCCAG | 13813 | GGGCCACCATCACAGCGCCAG |
| 8149 | GGGCCACUGAAUGUUUUCAGU | 13814 | GGGCCACTGAATGTTTTCAGT |
| 8150 | GGGCCAGCCUCUUUUCAUCCU | 13815 | GGGCCAGCCTCTTTTCATCCT |
| 8151 | GGGCCAGGGGGUCCCGUUGGU | 13816 | GGGCCAGGGGGTCCCGTTGGT |
| 8152 | GGGCCCAGGAUAACGGAGGGC | 13817 | GGGCCCAGGATAACGGAGGGC |
| 8153 | GGGCCCCAUGCUCGCAGCCUC | 13818 | GGGCCCCATGCTCGCAGCCTC |
| 8154 | GGGCCCCGAGAAUAGUCACUG | 13819 | GGGCCCCGAGAATAGTCACTG |
| 8155 | GGGCCCCUCUGUGAACUUGAU | 13820 | GGGCCCCTCTGTGAACTTGAT |
| 8156 | GGGCCUGACAUCCCCUCCCCU | 13821 | GGGCCTGACATCCCCTCCCCT |
| 8157 | GGGCCUGGAUGUCUCUGCUGA | 13822 | GGGCCTGGATGTCTCTGCTGA |
| 8158 | GGGCCUGUUUCUCUUAUCCUU | 13823 | GGGCCTGTTTCTCTTATCCTT |
| 8159 | GGGCGACAGGUGGCCACAGCG | 13824 | GGGCGACAGGTGGCCACAGCG |
| 8160 | GGGCGACAGGUGGCCACAGGA | 13825 | GGGCGACAGGTGGCCACAGGA |
| 8161 | GGGCGACUCGUGUAUGUCGCG | 13826 | GGGCGACTCGTGTATGTCGCG |
| 8162 | GGGCGAGGGCGCCCCCCAGGA | 13827 | GGGCGAGGGCGCCCCCCAGGA |
| 8163 | GGGCGAGUGGGGGUUGAUCUG | 13828 | GGGCGAGTGGGGGTTGATCTG |
| 8164 | GGGCGCCCCCCAGGACAAGGU | 13829 | GGGCGCCCCCCAGGACAAGGT |
| 8165 | GGGCGGACAUAAAGACGACAA | 13830 | GGGCGGACATAAAGACGACAA |
| 8166 | GGGCGGGGAGAUUCUGAGGCA | 13831 | GGGCGGGGAGATTCTGAGGCA |
| 8167 | GGGCUACUGUCCCCUUGGAAC | 13832 | GGGCTACTGTCCCCTTGGAAC |
| 8168 | GGGCUCAGGCGAUCCUCCUGC | 13833 | GGGCTCAGGCGATCCTCCTGC |
| 8169 | GGGCUCUGUCAAGCUGGGUGC | 13834 | GGGCTCTGTCAAGCTGGGTGC |
| 8170 | GGGCUGCUGAUGCACCAUGCA | 13835 | GGGCTGCTGATGCACCATGCA |
| 8171 | GGGCUGGUGGGGCGAGGGCGC | 13836 | GGGCTGGTGGGGCGAGGGCGC |
| 8172 | GGGCUUCUUCUCAUUUCCUCU | 13837 | GGGCTTCTTCTCATTTCCTCT |
| 8173 | GGGCUUUCCCUGGCCUGGGAU | 13838 | GGGCTTTCCCTGGCCTGGGAT |
| 8174 | GGGGAAGUGAAUGGCUUGGAG | 13839 | GGGGAAGTGAATGGCTTGGAG |
| 8175 | GGGGACAAUUGCCAAUCCCUU | 13840 | GGGGACAATTGCCAATCCCTT |
| 8176 | GGGGACACAGUGUUUUGUCCC | 13841 | GGGGACACAGTGTTTTGTCCC |
| 8177 | GGGGACAGCCUUGCUCGUCUG | 13842 | GGGGACAGCCTTGCTCGTCTG |
| 8178 | GGGGACAGUAGGUUUUCAGCC | 13843 | GGGGACAGTAGGTTTTCAGCC |
| 8179 | GGGGACAUCAUUCUCCCAAAA | 13844 | GGGGACATCATTCTCCCAAAA |
| 8180 | GGGGACCCGGGACUUGUCGUC | 13845 | GGGGACCCGGGACTTGTCGTC |
| 8181 | GGGGACGGAGGUGUCGCCACA | 13846 | GGGGACGGAGGTGTCGCCACA |
| 8182 | GGGGACUUCGCCAGGAGGGCC | 13847 | GGGGACTTCGCCAGGAGGGCC |
| 8183 | GGGGAGAUUCUGAGGCAACGU | 13848 | GGGGAGATTCTGAGGCAACGT |
| 8184 | GGGGAGCACGUCUCCUGGGAC | 13849 | GGGGAGCACGTCTCCTGGGAC |
| 8185 | GGGGAGGACAAUGGACAGAGC | 13850 | GGGGAGGACAATGGACAGAGC |
| 8186 | GGGGAUGAGGCUGGUGUACUC | 13851 | GGGGATGAGGCTGGTGTACTC |
| 8187 | GGGGAUGCAGGUGGAGCUGUU | 13852 | GGGGATGCAGGTGGAGCTGTT |
| 8188 | GGGGCACAGGCACUCGUAGCC | 13853 | GGGGCACAGGCACTCGTAGCC |
| 8189 | GGGGCAGGGAGGCACAGAUAC | 13854 | GGGGCAGGGAGGCACAGATAC |
| 8190 | GGGGCCACCAUCACAGCGCCA | 13855 | GGGGCCACCATCACAGCGCCA |
| 8191 | GGGGCCCCUCUGUGAACUUGA | 13856 | GGGGCCCCTCTGTGAACTTGA |
| 8192 | GGGGCCUGGAUGUCUCUGCUG | 13857 | GGGGCCTGGATGTCTCTGCTG |
| 8193 | GGGGCGAGGGCGCCCCCCAGG | 13858 | GGGGCGAGGGCGCCCCCCAGG |
| 8194 | GGGGGGGGGAGAUUCUGAGGC | 13859 | GGGGGGGGGAGATTCTGAGGC |
| 8195 | GGGGCUACUGUCCCCUUGGAA | 13860 | GGGGCTACTGTCCCCTTGGAA |
| 8196 | GGGGCUGGUGGGGCGAGGGCG | 13861 | GGGGCTGGTGGGGCGAGGGCG |
| 8197 | GGGGGACACAGUGUUUUGUCC | 13862 | GGGGGACACAGTGTTTTGTCC |
| 8198 | GGGGGACAGCCUUGCUCGUCU | 13863 | GGGGGACAGCCTTGCTCGTCT |
| 8199 | GGGGGAGCACGUCUCCUGGGA | 13864 | GGGGGAGCACGTCTCCTGGGA |
| 8200 | GGGGGAUGCAGGUGGAGCUGU | 13865 | GGGGGATGCAGGTGGAGCTGT |
| 8201 | GGGGGCCCCUCUGUGAACUUG | 13866 | GGGGGCCCCTCTGTGAACTTG |
| 8202 | GGGGGCCUGGAUGUCUCUGCU | 13867 | GGGGGCCTGGATGTCTCTGCT |
| 8203 | GGGGGGACACAGUGUUUUGUC | 13868 | GGGGGGACACAGTGTTTTGTC |
| 8204 | GGGGGGACAGCCUUGCUCGUC | 13869 | GGGGGGACAGCCTTGCTCGTC |
| 8205 | GGGGGGGACACAGUGUUUUGU | 13870 | GGGGGGGACACAGTGTTTTGT |
| 8206 | GGGGGUCCAGCUGGAAGCCUU | 13871 | GGGGGTCCAGCTGGAAGCCTT |
| 8207 | GGGGGUCCCGUUGGUGCCAUC | 13872 | GGGGGTCCCGTTGGTGCCATC |
| 8208 | GGGGGUGACAGGGCAAAGGCU | 13873 | GGGGGTGACAGGGCAAAGGCT |
| 8209 | GGGGGUUGAUCUGCGGGGCAG | 13874 | GGGGGTTGATCTGCGGGGCAG |
| 8210 | GGGGUAGCUGUAGCCGUCCUG | 13875 | GGGGTAGCTGTAGCCGTCCTG |
| 8211 | GGGGUCCAGCUGGAAGCCUUC | 13876 | GGGGTCCAGCTGGAAGCCTTC |
| 8212 | GGGGUCCCGUUGGUGCCAUCU | 13877 | GGGGTCCCGTTGGTGCCATCT |
| 8213 | GGGGUCGGGAGGAUGGGCACC | 13878 | GGGGTCGGGAGGATGGGCACC |
| 8214 | GGGGUCGUUGUCGCAGGCCCA | 13879 | GGGGTCGTTGTCGCAGGCCCA |
| 8215 | GGGGUCUUACUAUGUUGCCCA | 13880 | GGGGTCTTACTATGTTGCCCA |
| 8216 | GGGGUGACAGGGCAAAGGCUA | 13881 | GGGGTGACAGGGCAAAGGCTA |
| 8217 | GGGGUGGCCCCAGGCAGCCGG | 13882 | GGGGTGGCCCCAGGCAGCCGG |
| 8218 | GGGGUGGGCCAGCCUCUUUUC | 13883 | GGGGTGGGCCAGCCTCTTTTC |
| 8219 | GGGGUUGAUCUGCGGGGCAGG | 13884 | GGGGTTGATCTGCGGGGCAGG |
| 8220 | GGGGUUGUCAAAGUUGAUGCU | 13885 | GGGGTTGTCAAAGTTGATGCT |
| 8221 | GGGGUUUCACGUGUUAGCCAG | 13886 | GGGGTTTCACGTGTTAGCCAG |
| 8222 | GGGGUUUGGCUUAGAGAUUGG | 13887 | GGGGTTTGGCTTAGAGATTGG |
| 8223 | GGGUAGCUGUAGCCGUCCUGG | 13888 | GGGTAGCTGTAGCCGTCCTGG |
| 8224 | GGGUAGGGGUCGGGAGGAUGG | 13889 | GGGTAGGGGTCGGGAGGATGG |
| 8225 | GGGUCAUGAUUCGGGGGUGAC | 13890 | GGGTCATGATTCGGGGGTGAC |
| 8226 | GGGUCCAGCUGGAAGCCUUCC | 13891 | GGGTCCAGCTGGAAGCCTTCC |
| 8227 | GGGUCCCGUUGGUGCCAUCUG | 13892 | GGGTCCCGTTGGTGCCATCTG |
| 8228 | GGGUCCCUCGCAGAGUGUCAC | 13893 | GGGTCCCTCGCAGAGTGTCAC |
| 8229 | GGGUCCCUCGCAGAGUGUCAG | 13894 | GGGTCCCTCGCAGAGTGTCAG |
| 8230 | GGGUCGGGAGGAUGGGCACCA | 13895 | GGGTCGGGAGGATGGGCACCA |
| 8231 | GGGUCGUUGUCGCAGGCCCAC | 13896 | GGGTCGTTGTCGCAGGCCCAC |
| 8232 | GGGUCUUACUAUGUUGCCCAG | 13897 | GGGTCTTACTATGTTGCCCAG |
| 8233 | GGGUCUUCAUCUGUUGCCCAG | 13898 | GGGTCTTCATCTGTTGCCCAG |
| 8234 | GGGUGACAGGGCAAAGGCUAA | 13899 | GGGTGACAGGGCAAAGGCTAA |
| 8235 | GGGUGACCAGUGACUCAGGCU | 13900 | GGGTGACCAGTGACTCAGGCT |
| 8236 | GGGUGAGGUUGUGGAAGAGAA | 13901 | GGGTGAGGTTGTGGAAGAGAA |
| 8237 | GGGUGAUGCAUUCGCCGCUGU | 13902 | GGGTGATGCATTCGCCGCTGT |
| 8238 | GGGUGAUGCCAUUGGGCCACU | 13903 | GGGTGATGCCATTGGGCCACT |
| 8239 | GGGUGCAGGCCACGUGUCCUC | 13904 | GGGTGCAGGCCACGTGTCCTC |
| 8240 | GGGUGCUGCAGAUCAUUCUCU | 13905 | GGGTGCTGCAGATCATTCTCT |
| 8241 | GGGUGGCCACUGCAGCCUCAG | 13906 | GGGTGGCCACTGCAGCCTCAG |
| 8242 | GGGUGGCCCCAGGCAGCCGGG | 13907 | GGGTGGCCCCAGGCAGCCGGG |
| 8243 | GGGUGGCCGGGGGCCCCUCUG | 13908 | GGGTGGCCGGGGGCCCCTCTG |
| 8244 | GGGUGGGCCAGCCUCUUUUCA | 13909 | GGGTGGGCCAGCCTCTTTTCA |
| 8245 | GGGUGGGUCAUGAUUCGGGGG | 13910 | GGGTGGGTCATGATTCGGGGG |
| 8246 | GGGUGGUCCUCUCACACCAGU | 13911 | GGGTGGTCCTCTCACACCAGT |
| 8247 | GGGUGGUUGUGUGCUGUGUCC | 13912 | GGGTGGTTGTGTGCTGTGTCC |
| 8248 | GGGUGUCUCAGGCACUUAAUA | 13913 | GGGTGTCTCAGGCACTTAATA |
| 8249 | GGGUUCACACCAUUCUCCUGC | 13914 | GGGTTCACACCATTCTCCTGC |
| 8250 | GGGUUCAUCUGACCAGUCCCG | 13915 | GGGTTCATCTGACCAGTCCCG |
| 8251 | GGGUUCUGGGCAGGGACGGGA | 13916 | GGGTTCTGGGCAGGGACGGGA |
| 8252 | GGGUUGAUCUGCGGGGCAGGG | 13917 | GGGTTGATCTGCGGGGCAGGG |
| 8253 | GGGUUGUCAAAGUUGAUGCUG | 13918 | GGGTTGTCAAAGTTGATGCTG |
| 8254 | GGGUUUCACGUGUUAGCCAGG | 13919 | GGGTTTCACGTGTTAGCCAGG |
| 8255 | GGGUUUGCAAAAAUAAAUAGA | 13920 | GGGTTTGCAAAAATAAATAGA |
| 8256 | GGGUUUGGCUUAGAGAUUGGU | 13921 | GGGTTTGGCTTAGAGATTGGT |
| 8257 | GGUAAACACGUUGACUCCUGA | 13922 | GGTAAACACGTTGACTCCTGA |
| 8258 | GGUAAACUUGGGCGAGUGGGG | 13923 | GGTAAACTTGGGCGAGTGGGG |
| 8259 | GGUAACCGGGUGUCUCAGGCA | 13924 | GGTAACCGGGTGTCTCAGGCA |
| 8260 | GGUACAAACCCACCUCGAAAG | 13925 | GGTACAAACCCACCTCGAAAG |
| 8261 | GGUAGCUGUAGCCGUCCUGGU | 13926 | GGTAGCTGTAGCCGTCCTGGT |
| 8262 | GGUAGGCGAUGGAGCCCACAG | 13927 | GGTAGGCGATGGAGCCCACAG |
| 8263 | GGUAGGGGUCGGGAGGAUGGG | 13928 | GGTAGGGGTCGGGAGGATGGG |
| 8264 | GGUAUCCGCAACAGAGACAGU | 13929 | GGTATCCGCAACAGAGACAGT |
| 8265 | GGUCAACCUGCCCCUCUCUGU | 13930 | GGTCAACCTGCCCCTCTCTGT |
| 8266 | GGUCACACUGCCGGCUGCCAU | 13931 | GGTCACACTGCCGGCTGCCAT |
| 8267 | GGUCAGACCAGUAGAUUCUAU | 13932 | GGTCAGACCAGTAGATTCTAT |
| 8268 | GGUCAUGAUUCGGGGGUGACA | 13933 | GGTCATGATTCGGGGGTGACA |
| 8269 | GGUCAUUGCAGACGUGGGAAC | 13934 | GGTCATTGCAGACGTGGGAAC |
| 8270 | GGUCCAGCGCAAUUUCCAGCC | 13935 | GGTCCAGCGCAATTTCCAGCC |
| 8271 | GGUCCAGCGUCAUCUUCCUGA | 13936 | GGTCCAGCGTCATCTTCCTGA |
| 8272 | GGUCCAGCUGGAAGCCUUCCU | 13937 | GGTCCAGCTGGAAGCCTTCCT |
| 8273 | GGUCCAGUAGAUGUUGCUGUG | 13938 | GGTCCAGTAGATGTTGCTGTG |
| 8274 | GGUCCCCCCGGGCCUGUUUCU | 13939 | GGTCCCCCCGGGCCTGTTTCT |
| 8275 | GGUCCCGCACUCUUUGAUGGG | 13940 | GGTCCCGCACTCTTTGATGGG |
| 8276 | GGUCCCGUUGGUGCCAUCUGC | 13941 | GGTCCCGTTGGTGCCATCTGC |
| 8277 | GGUCCCUCGCAGAGUGUCACA | 13942 | GGTCCCTCGCAGAGTGTCACA |
| 8278 | GGUCCCUCGCAGAGUGUCAGA | 13943 | GGTCCCTCGCAGAGTGTCAGA |
| 8279 | GGUCCUCUCACACCAGUUCAC | 13944 | GGTCCTCTCACACCAGTTCAC |
| 8280 | GGUCGGGAGGAUGGGCACCAG | 13945 | GGTCGGGAGGATGGGCACCAG |
| 8281 | GGUCGGGUGGUUGUGUGCUGU | 13946 | GGTCGGGTGGTTGTGTGCTGT |
| 8282 | GGUCGUUGUCGCAGGCCCACA | 13947 | GGTCGTTGTCGCAGGCCCACA |
| 8283 | GGUCUCAGGAAGGGUUCUGGG | 13948 | GGTCTCAGGAAGGGTTCTGGG |
| 8284 | GGUCUCCAACUCCUGAACUCA | 13949 | GGTCTCCAACTCCTGAACTCA |
| 8285 | GGUCUCGAUCUCCUGACCUCA | 13950 | GGTCTCGATCTCCTGACCTCA |
| 8286 | GGUCUCGGUGCCACCACGGAU | 13951 | GGTCTCGGTGCCACCACGGAT |
| 8287 | GGUCUGAGUCACAGACGAACU | 13952 | GGTCTGAGTCACAGACGAACT |
| 8288 | GGUCUUACUAUGUUGCCCAGG | 13953 | GGTCTTACTATGTTGCCCAGG |
| 8289 | GGUCUUCAUCUGUUGCCCAGG | 13954 | GGTCTTCATCTGTTGCCCAGG |
| 8290 | GGUCUUCCGGUUGCCCCCGUU | 13955 | GGTCTTCCGGTTGCCCCCGTT |
| 8291 | GGUCUUCUCUGUCUUUGAAUA | 13956 | GGTCTTCTCTGTCTTTGAATA |
| 8292 | GGUCUUCUGAUAGACGGGGUU | 13957 | GGTCTTCTGATAGACGGGGTT |
| 8293 | GGUGAAGAAGAGGUAGGCGAU | 13958 | GGTGAAGAAGAGGTAGGCGAT |
| 8294 | GGUGAAUGAGUUUGGUCUCGG | 13959 | GGTGAATGAGTTTGGTCTCGG |
| 8295 | GGUGACAGACAAGCACGUCUC | 13960 | GGTGACAGACAAGCACGTCTC |
| 8296 | GGUGACAGGGCAAAGGCUAAC | 13961 | GGTGACAGGGCAAAGGCTAAC |
| 8297 | GGUGACCAGUGACUCAGGCUC | 13962 | GGTGACCAGTGACTCAGGCTC |
| 8298 | GGUGAGACAUUGUCACUAUCU | 13963 | GGTGAGACATTGTCACTATCT |
| 8299 | GGUGAGCACCGGGCAGGAGGC | 13964 | GGTGAGCACCGGGCAGGAGGC |
| 8300 | GGUGAGCCCAGGGGUGGCCCC | 13965 | GGTGAGCCCAGGGGTGGCCCC |
| 8301 | GGUGAGGUUGUGGAAGAGAAC | 13966 | GGTGAGGTTGTGGAAGAGAAC |
| 8302 | GGUGAUGCAUUCGCCGCUGUG | 13967 | GGTGATGCATTCGCCGCTGTG |
| 8303 | GGUGAUGCCAUUGGGCCACUG | 13968 | GGTGATGCCATTGGGCCACTG |
| 8304 | GGUGCAGGCCACGUGUCCUCA | 13969 | GGTGCAGGCCACGTGTCCTCA |
| 8305 | GGUGCCACCACGGAUUCAGCC | 13970 | GGTGCCACCACGGATTCAGCC |
| 8306 | GGUGCCAUCUGCUGUUGUGUG | 13971 | GGTGCCATCTGCTGTTGTGTG |
| 8307 | GGUGCCCGCCACCACGCCCGG | 13972 | GGTGCCCGCCACCACGCCCGG |
| 8308 | GGUGCUGCAGAUCAUUCUCUG | 13973 | GGTGCTGCAGATCATTCTCTG |
| 8309 | GGUGGAGAGAAACUCAAAACU | 13974 | GGTGGAGAGAAACTCAAAACT |
| 8310 | GGUGGAGCUGUUGCACUGGAA | 13975 | GGTGGAGCTGTTGCACTGGAA |
| 8311 | GGUGGAUGAGGCAGGGCCAGG | 13976 | GGTGGATGAGGCAGGGCCAGG |
| 8312 | GGUGGAUGUCUCCUGGGUGGC | 13977 | GGTGGATGTCTCCTGGGTGGC |
| 8313 | GGUGGCCACAGCGCAGUUUUC | 13978 | GGTGGCCACAGCGCAGTTTTC |
| 8314 | GGUGGCCACAGGACAGCCUUG | 13979 | GGTGGCCACAGGACAGCCTTG |
| 8315 | GGUGGCCACUGCAGCCUCAGC | 13980 | GGTGGCCACTGCAGCCTCAGC |
| 8316 | GGUGGCCCCAGGCAGCCGGGA | 13981 | GGTGGCCCCAGGCAGCCGGGA |
| 8317 | GGUGGCCGGGGGCCCCUCUGU | 13982 | GGTGGCCGGGGGCCCCTCTGT |
| 8318 | GGUGGGCCAGCCUCUUUUCAU | 13983 | GGTGGGCCAGCCTCTTTTCAT |
| 8319 | GGUGGGCGGACAUAAAGACGA | 13984 | GGTGGGCGGACATAAAGACGA |
| 8320 | GGUGGGGACGGAGGUGUCGCC | 13985 | GGTGGGGACGGAGGTGTCGCC |
| 8321 | GGUGGGGCGAGGGCGCCCCCC | 13986 | GGTGGGGCGAGGGCGCCCCCC |
| 8322 | GGUGGGUCAUGAUUCGGGGGU | 13987 | GGTGGGTCATGATTCGGGGGT |
| 8323 | GGUGGUCCUCUCACACCAGUU | 13988 | GGTGGTCCTCTCACACCAGTT |
| 8324 | GGUGGUUGUGUGCUGUGUCCU | 13989 | GGTGGTTGTGTGCTGTGTCCT |
| 8325 | GGUGUACUCGCUCCGGUCCAG | 13990 | GGTGTACTCGCTCCGGTCCAG |
| 8326 | GGUGUCAUAGGAAGAGACGCC | 13991 | GGTGTCATAGGAAGAGACGCC |
| 8327 | GGUGUCAUCCUGGUCCCCCCG | 13992 | GGTGTCATCCTGGTCCCCCCG |
| 8328 | GGUGUCCUGACAGCCACACCU | 13993 | GGTGTCCTGACAGCCACACCT |
| 8329 | GGUGUCGCCACAGAGCACAGC | 13994 | GGTGTCGCCACAGAGCACAGC |
| 8330 | GGUGUCGGGAACAGGUCGGGU | 13995 | GGTGTCGGGAACAGGTCGGGT |
| 8331 | GGUGUCGGGAUCCUGACACUC | 13996 | GGTGTCGGGATCCTGACACTC |
| 8332 | GGUGUCUCAGGCACUUAAUAA | 13997 | GGTGTCTCAGGCACTTAATAA |
| 8333 | GGUUCACACCAUUCUCCUGCC | 13998 | GGTTCACACCATTCTCCTGCC |
| 8334 | GGUUCACGCAGAGCUGGCUGC | 13999 | GGTTCACGCAGAGCTGGCTGC |
| 8335 | GGUUCAUCUGACCAGUCCCGG | 14000 | GGTTCATCTGACCAGTCCCGG |
| 8336 | GGUUCAUUUAAGAAAUUUAAU | 14001 | GGTTCATTTAAGAAATTTAAT |
| 8337 | GGUUCCCUGCACUGGGGGGGA | 14002 | GGTTCCCTGCACTGGGGGGGA |
| 8338 | GGUUCUGGGCAGGGACGGGAC | 14003 | GGTTCTGGGCAGGGACGGGAC |
| 8339 | GGUUGACACGGCCCCCACAGC | 14004 | GGTTGACACGGCCCCCACAGC |
| 8340 | GGUUGAUCUGCGGGGCAGGGA | 14005 | GGTTGATCTGCGGGGCAGGGA |
| 8341 | GGUUGCCCCCGUUGACAUCGA | 14006 | GGTTGCCCCCGTTGACATCGA |
| 8342 | GGUUGGCACUGAAAAUGGCUU | 14007 | GGTTGGCACTGAAAATGGCTT |
| 8343 | GGUUGGGGAUGAGGCUGGUGU | 14008 | GGTTGGGGATGAGGCTGGTGT |
| 8344 | GGUUGGUGAAGAAGAGGUAGG | 14009 | GGTTGGTGAAGAAGAGGTAGG |
| 8345 | GGUUGUCAAAGUUGAUGCUGU | 14010 | GGTTGTCAAAGTTGATGCTGT |
| 8346 | GGUUGUGGAAGAGAACCAUAU | 14011 | GGTTGTGGAAGAGAACCATAT |
| 8347 | GGUUGUGGCAAAUGUGGACCU | 14012 | GGTTGTGGCAAATGTGGACCT |
| 8348 | GGUUGUGUGCUGUGUCCUUAC | 14013 | GGTTGTGTGCTGTGTCCTTAC |
| 8349 | GGUUUAAAAAGUGACACCCAU | 14014 | GGTTTAAAAAGTGACACCCAT |
| 8350 | GGUUUCACGUGUUAGCCAGGA | 14015 | GGTTTCACGTGTTAGCCAGGA |
| 8351 | GGUUUGCAAAAAUAAAUAGAU | 14016 | GGTTTGCAAAAATAAATAGAT |
| 8352 | GGUUUGGCUUAGAGAUUGGUG | 14017 | GGTTTGGCTTAGAGATTGGTG |
| 8353 | GGUUUUCAGCCAACAAGUUGA | 14018 | GGTTTTCAGCCAACAAGTIGA |
| 8354 | GGUUUUUGUAGAGAUGGGAUC | 14019 | GGTTTTTGTAGAGATGGGATC |
| 8355 | GGUUUUUUUUUGUUUUUUUUU | 14020 | GGTTTTTTTTTGTTTTTTTTT |
| 8356 | GUAAACACGUUGACUCCUGAG | 14021 | GTAAACACGTTGACTCCTGAG |
| 8357 | GUAAACUUGGGCGAGUGGGGG | 14022 | GTAAACTTGGGCGAGTGGGGG |
| 8358 | GUAAAGACCCCUACAGCGCUG | 14023 | GTAAAGACCCCTACAGCGCTG |
| 8359 | GUAACACGGCGAUUUCAGAAA | 14024 | GTAACACGGCGATTTCAGAAA |
| 8360 | GUAACCGGGUGUCUCAGGCAC | 14025 | GTAACCGGGTGTCTCAGGCAC |
| 8361 | GUAAGACAUUCAGGCUACAAA | 14026 | GTAAGACATTCAGGCTACAAA |
| 8362 | GUAAGGAACCGAGGCCUUGAG | 14027 | GTAAGGAACCGAGGCCTTGAG |
| 8363 | GUACAAACCCACCUCGAAAGA | 14028 | GTACAAACCCACCTCGAAAGA |
| 8364 | GUACAACUCUGAACUGAGAAA | 14029 | GTACAACTCTGAACTGAGAAA |
| 8365 | GUACACAGUGUACAACUCUGA | 14030 | GTACACAGTGTACAACTCTGA |
| 8366 | GUACAGGGACGCAUUUACGUG | 14031 | GTACAGGGACGCATTTACGTG |
| 8367 | GUACAUAAAGAAGAGGCAUUU | 14032 | GTACATAAAGAAGAGGCATTT |
| 8368 | GUACAUGAAGCCAUGAACAGG | 14033 | GTACATGAAGCCATGAACAGG |
| 8369 | GUACCCAGCUGAUUUUUAACU | 14034 | GTACCCAGCTGATTTTTAACT |
| 8370 | GUACUCGCUCCGGUCCAGCGU | 14035 | GTACTCGCTCCGGTCCAGCGT |
| 8371 | GUAGAGACGGGGUUUCACGUG | 14036 | GTAGAGACGGGGTTTCACGTG |
| 8372 | GUAGAGAUGGGAUCUCGCUUU | 14037 | GTAGAGATGGGATCTCGCTTT |
| 8373 | GUAGAGGCGGCCACUGAGGAG | 14038 | GTAGAGGCGGCCACTGAGGAG |
| 8374 | GUAGAUGUCCACACCAUUCAG | 14039 | GTAGATGTCCACACCATTCAG |
| 8375 | GUAGAUGUUGCUGUGGAUCCA | 14040 | GTAGATGTTGCTGTGGATCCA |
| 8376 | GUAGAUUCUAUUGCUGGCCAC | 14041 | GTAGATTCTATTGCTGGCCAC |
| 8377 | GUAGCCACCCUCCAGGUUCAC | 14042 | GTAGCCACCCTCCAGGTTCAC |
| 8378 | GUAGCCGAUCUUAAGGUCAUU | 14043 | GTAGCCGATCTTAAGGTCATT |
| 8379 | GUAGCCGUCCUGGUUGUGGCA | 14044 | GTAGCCGTCCTGGTTGTGGCA |
| 8380 | GUAGCUGGGACUACAGGUGCC | 14045 | GTAGCTGGGACTACAGGTGCC |
| 8381 | GUAGCUGGGAUCACAGGCACG | 14046 | GTAGCTGGGATCACAGGCACG |
| 8382 | GUAGCUGUAGCCGUCCUGGUU | 14047 | GTAGCTGTAGCCGTCCTGGTT |
| 8383 | GUAGGAGAUGCAUUUCCCGUC | 14048 | GTAGGAGATGCATTTCCCGTC |
| 8384 | GUAGGCGAUGGAGCCCACAGC | 14049 | GTAGGCGATGGAGCCCACAGC |
| 8385 | GUAGGGAGAGGCACGCUCUGU | 14050 | GTAGGGAGAGGCACGCTCTGT |
| 8386 | GUAGGGGUCGGGAGGAUGGGC | 14051 | GTAGGGGTCGGGAGGATGGGC |
| 8387 | GUAGGUUUUCAGCCAACAAGU | 14052 | GTAGGTTTTCAGCCAACAAGT |
| 8388 | GUAUAUAUAUAUAUAUAAGUG | 14053 | GTATATATATATATATAAGTG |
| 8389 | GUAUCCGCAACAGAGACAGUG | 14054 | GTATCCGCAACAGAGACAGTG |
| 8390 | GUAUGUCGCGGAGGCGGCUCC | 14055 | GTATGTCGCGGAGGCGGCTCC |
| 8391 | GUAUUUCUGAAAUAGGGUCUU | 14056 | GTATTTCTGAAATAGGGTCTT |
| 8392 | GUAUUUUUAGUAGAGACGGGG | 14057 | GTATTTTTAGTAGAGACGGGG |
| 8393 | GUCAAACGAUCCAGACUGGAG | 14058 | GTCAAACGATCCAGACTGGAG |
| 8394 | GUCAAAGUUGAUGCUGUUGAU | 14059 | GTCAAAGTTGATGCTGTTGAT |
| 8395 | GUCAACCCAGUAGAGGCGGCC | 14060 | GTCAACCCAGTAGAGGCGGCC |
| 8396 | GUCAACCUGCCCCUCUCUGUC | 14061 | GTCAACCTGCCCCTCTCTGTC |
| 8397 | GUCAAGCUGGGUGCUGCAGAU | 14062 | GTCAAGCTGGGTGCTGCAGAT |
| 8398 | GUCACACAGCUGCCCCCGAGA | 14063 | GTCACACAGCTGCCCCCGAGA |
| 8399 | GUCACACUGCCGGCUGCCAUG | 14064 | GTCACACTGCCGGCTGCCATG |
| 8400 | GUCACAGACGAACUGCCGAGA | 14065 | GTCACAGACGAACTGCCGAGA |
| 8401 | GUCACAUUAACGCAGCCAACU | 14066 | GTCACATTAACGCAGCCAACT |
| 8402 | GUCACCAGCGAGUAGAUGUCC | 14067 | GTCACCAGCGAGTAGATGTCC |
| 8403 | GUCACGGUGGAGAGAAACUCA | 14068 | GTCACGGTGGAGAGAAACTCA |
| 8404 | GUCACUAUCUCCACCGUGGUG | 14069 | GTCACTATCTCCACCGTGGTG |
| 8405 | GUCACUGAACAAAUACAGCAA | 14070 | GTCACTGAACAAATACAGCAA |
| 8406 | GUCACUGUCUCUCCGGACAUC | 14071 | GTCACTGTCTCTCCGGACATC |
| 8407 | GUCAGACAGCCUUGCUCGUCU | 14072 | GTCAGACAGCCTTGCTCGTCT |
| 8408 | GUCAGACCAGUAGAUUCUAUU | 14073 | GTCAGACCAGTAGATTCTATT |
| 8409 | GUCAGAUUUGUCCUUGCAGUC | 14074 | GTCAGATTTGTCCTTGCAGTC |
| 8410 | GUCAGGGCGACAGGUGGCCAC | 14075 | GTCAGGGCGACAGGTGGCCAC |
| 8411 | GUCAGUCCAGUACAUGAAGCC | 14076 | GTCAGTCCAGTACATGAAGCC |
| 8412 | GUCAUAGGAAGAGACGCCGUG | 14077 | GTCATAGGAAGAGACGCCGTG |
| 8413 | GUCAUAUUCCCGGUCACACUG | 14078 | GTCATATTCCCGGTCACACTG |
| 8414 | GUCAUCCUCCAGACUGACCAU | 14079 | GTCATCCTCCAGACTGACCAT |
| 8415 | GUCAUCCUGGUCCCCCCGGGC | 14080 | GTCATCCTGGTCCCCCCGGGC |
| 8416 | GUCAUCUUCCUGACCUCGUGC | 14081 | GTCATCTTCCTGACCTCGTGC |
| 8417 | GUCAUGAUUCGGGGGUGACAG | 14082 | GTCATGATTCGGGGGTGACAG |
| 8418 | GUCAUUGCAGACGUGGGAACA | 14083 | GTCATTGCAGACGTGGGAACA |
| 8419 | GUCCAAAAUACUUUGUCCUCA | 14084 | GTCCAAAATACTTTGTCCTCA |
| 8420 | GUCCAAGCAGUCCCGGUCUGA | 14085 | GTCCAAGCAGTCCCGGTCTGA |
| 8421 | GUCCAAGCAUUCGUUGGUCCC | 14086 | GTCCAAGCATTCGTTGGTCCC |
| 8422 | GUCCACACCAUUCAGGCCCCC | 14087 | GTCCACACCATTCAGGCCCCC |
| 8423 | GUCCACAGCCAGCCCGUCGGG | 14088 | GTCCACAGCCAGCCCGTCGGG |
| 8424 | GUCCACUUGGCCAUCGCACCU | 14089 | GTCCACTTGGCCATCGCACCT |
| 8425 | GUCCAGAGCGACCACGUUCCU | 14090 | GTCCAGAGCGACCACGTTCCT |
| 8426 | GUCCAGCGCAAUUUCCAGCCC | 14091 | GTCCAGCGCAATTTCCAGCCC |
| 8427 | GUCCAGCGUCAUCUUCCUGAC | 14092 | GTCCAGCGTCATCTTCCTGAC |
| 8428 | GUCCAGCUGGAAGCCUUCCUC | 14093 | GTCCAGCTGGAAGCCTTCCTC |
| 8429 | GUCCAGGGUGAUGCAUUCGCC | 14094 | GTCCAGGGTGATGCATTCGCC |
| 8430 | GUCCAGUACAUGAAGCCAUGA | 14095 | GTCCAGTACATGAAGCCATGA |
| 8431 | GUCCAGUAGAUGUUGCUGUGG | 14096 | GTCCAGTAGATGTTGCTGTGG |
| 8432 | GUCCAGUUCGUGCAAAUAAUC | 14097 | GTCCAGTTCGTGCAAATAATC |
| 8433 | GUCCCCCCGGGCCUGUUUCUC | 14098 | GTCCCCCCGGGCCTGTTTCTC |
| 8434 | GUCCCCGCCGCGGCGAGGAGC | 14099 | GTCCCCGCCGCGGCGAGGAGC |
| 8435 | GUCCCCGGAUUUGCAGGUGAC | 14100 | GTCCCCGGATTTGCAGGTGAC |
| 8436 | GUCCCCGGCAUUCAUUGACAC | 14101 | GTCCCCGGCATTCATTGACAC |
| 8437 | GUCCCCUUGGAACACGUAAAG | 14102 | GTCCCCTTGGAACACGTAAAG |
| 8438 | GUCCCGACCCGGAUCACGACC | 14103 | GTCCCGACCCGGATCACGACC |
| 8439 | GUCCCGCACUCUUUGAUGGGU | 14104 | GTCCCGCACTCTTTGATGGGT |
| 8440 | GUCCCGGCAGUCUCUAGCCAU | 14105 | GTCCCGGCAGTCTCTAGCCAT |
| 8441 | GUCCCGGUCAACCUGCCCCUC | 14106 | GTCCCGGTCAACCTGCCCCTC |
| 8442 | GUCCCGGUCUGAGUCACAGAC | 14107 | GTCCCGGTCTGAGTCACAGAC |
| 8443 | GUCCCGUCAAACGAUCCAGAC | 14108 | GTCCCGTCAAACGATCCAGAC |
| 8444 | GUCCCGUUGGUGCCAUCUGCU | 14109 | GTCCCGTTGGTGCCATCTGCT |
| 8445 | GUCCCUCGCAGAGUGUCACAU | 14110 | GTCCCTCGCAGAGTGTCACAT |
| 8446 | GUCCCUCGCAGAGUGUCAGAC | 14111 | GTCCCTCGCAGAGTGTCAGAC |
| 8447 | GUCCCUGGCCAGCAGCAUGCC | 14112 | GTCCCTGGCCAGCAGCATGCC |
| 8448 | GUCCCUGGGGACAAUUGCCAA | 14113 | GTCCCTGGGGACAATTGCCAA |
| 8449 | GUCCGAAGCCUGUUCUGCCUC | 14114 | GTCCGAAGCCTGTTCTGCCTC |
| 8450 | GUCCGGGCAGGCGCAGGUAAA | 14115 | GTCCGGGCAGGCGCAGGTAAA |
| 8451 | GUCCUAGCUGGAAACCCUGGC | 14116 | GTCCTAGCTGGAAACCCTGGC |
| 8452 | GUCCUCAAAGACGGCCAAGGA | 14117 | GTCCTCAAAGACGGCCAAGGA |
| 8453 | GUCCUCACGGCCAAGGUAACC | 14118 | GTCCTCACGGCCAAGGTAACC |
| 8454 | GUCCUCUCACACCAGUUCACU | 14119 | GTCCTCTCACACCAGTTCACT |
| 8455 | GUCCUGACAGCCACACCUGGG | 14120 | GTCCTGACAGCCACACCTGGG |
| 8456 | GUCCUGGGAGCACGUCUUGGG | 14121 | GTCCTGGGAGCACGTCTTGGG |
| 8457 | GUCCUGGUUGUGGCAAAUGUG | 14122 | GTCCTGGTTGTGGCAAATGTG |
| 8458 | GUCCUUACGGCUGUGGAGCUG | 14123 | GTCCTTACGGCTGTGGAGCTG |
| 8459 | GUCCUUGCAGUCAUAUUCCCG | 14124 | GTCCTTGCAGTCATATTCCCG |
| 8460 | GUCCUUGCAGUCGGGGCCACC | 14125 | GTCCTTGCAGTCGGGGCCACC |
| 8461 | GUCGCAGGCCCACAGCUGGGG | 14126 | GTCGCAGGCCCACAGCTGGGG |
| 8462 | GUCGCAGUCCACUUGGCCAUC | 14127 | GTCGCAGTCCACTTGGCCATC |
| 8463 | GUCGCCACAGAGCACAGCGGA | 14128 | GTCGCCACAGAGCACAGCGGA |
| 8464 | GUCGCCCACGUGGCUGAAGAU | 14129 | GTCGCCCACGTGGCTGAAGAT |
| 8465 | GUCGCCCACUGCAGUCCCCGC | 14130 | GTCGCCCACTGCAGTCCCCGC |
| 8466 | GUCGCCCAGAGCUUGGUGAGA | 14131 | GTCGCCCAGAGCTTGGTGAGA |
| 8467 | GUCGCCCAGGCCAGACUGCGG | 14132 | GTCGCCCAGGCCAGACTGCGG |
| 8468 | GUCGCCUUUGUGACAGGAACC | 14133 | GTCGCCTTTGTGACAGGAACC |
| 8469 | GUCGCGGAGGCGGCUCCGGGG | 14134 | GTCGCGGAGGCGGCTCCGGGG |
| 8470 | GUCGGGAACAGGUCGGGUGGU | 14135 | GTCGGGAACAGGTCGGGTGGT |
| 8471 | GUCGGGAGGAUGGGCACCAGG | 14136 | GTCGGGAGGATGGGCACCAGG |
| 8472 | GUCGGGAUCCUGACACUCAUC | 14137 | GTCGGGATCCTGACACTCATC |
| 8473 | GUCGGGGCACAGGCACUCGUA | 14138 | GTCGGGGCACAGGCACTCGTA |
| 8474 | GUCGGGGCCACCAUCACAGCG | 14139 | GTCGGGGCCACCATCACAGCG |
| 8475 | GUCGGGGGCCUGGAUGUCUCU | 14140 | GTCGGGGGCCTGGATGTCTCT |
| 8476 | GUCGGGGUCGUUGUCGCAGGC | 14141 | GTCGGGGTCGTTGTCGCAGGC |
| 8477 | GUCGGGUGGUUGUGUGCUGUG | 14142 | GTCGGGTGGTTGTGTGCTGTG |
| 8478 | GUCGGUCCAGUAGAUGUUGCU | 14143 | GTCGGTCCAGTAGATGTTGCT |
| 8479 | GUCGUCGCCUUUGUGACAGGA | 14144 | GTCGTCGCCTTTGTGACAGGA |
| 8480 | GUCGUUGUCGCAGGCCCACAG | 14145 | GTCGTTGTCGCAGGCCCACAG |
| 8481 | GUCUAGCAAGGCUUGCAUAGA | 14146 | GTCTAGCAAGGCTTGCATAGA |
| 8482 | GUCUCAGGAAGGGUUCUGGGC | 14147 | GTCTCAGGAAGGGTTCTGGGC |
| 8483 | GUCUCAGGCACUUAAUAAAUA | 14148 | GTCTCAGGCACTTAATAAATA |
| 8484 | GUCUCCAACUCCUGAACUCAA | 14149 | GTCTCCAACTCCTGAACTCAA |
| 8485 | GUCUCCUGGGACUCAUCAGAG | 14150 | GTCTCCTGGGACTCATCAGAG |
| 8486 | GUCUCCUGGGUGGCCACUGCA | 14151 | GTCTCCTGGGTGGCCACTGCA |
| 8487 | GUCUCGAGGGGUAGCUGUAGC | 14152 | GTCTCGAGGGGTAGCTGTAGC |
| 8488 | GUCUCGAUCUCCUGACCUCAU | 14153 | GTCTCGATCTCCTGACCTCAT |
| 8489 | GUCUCGCUCUGUCGCCCAGGC | 14154 | GTCTCGCTCTGTCGCCCAGGC |
| 8490 | GUCUCGGUGCCACCACGGAUU | 14155 | GTCTCGGTGCCACCACGGATT |
| 8491 | GUCUCUAGCCAUGUUGCAGAC | 14156 | GTCTCTAGCCATGTTGCAGAC |
| 8492 | GUCUCUCCGGACAUCAGUGCA | 14157 | GTCTCTCCGGACATCAGTGCA |
| 8493 | GUCUCUGCUGAUGACGGUGUC | 14158 | GTCTCTGCTGATGACGGTGTC |
| 8494 | GUCUGACGGAGGCUGUCACUG | 14159 | GTCTGACGGAGGCTGTCACTG |
| 8495 | GUCUGAGCCGUCCAAGCAGUC | 14160 | GTCTGAGCCGTCCAAGCAGTC |
| 8496 | GUCUGAGCCGUUGUCGCAGUC | 14161 | GTCTGAGCCGTTGTCGCAGTC |
| 8497 | GUCUGAGUCACAGACGAACUG | 14162 | GTCTGAGTCACAGACGAACTG |
| 8498 | GUCUGGCCAAAUUCAUUUUUU | 14163 | GTCTGGCCAAATTCATTTTTT |
| 8499 | GUCUUACUAUGUUGCCCAGGC | 14164 | GTCTTACTATGTTGCCCAGGC |
| 8500 | GUCUUCAUCUGUUGCCCAGGC | 14165 | GTCTTCATCTGTTGCCCAGGC |
| 8501 | GUCUUCCGGUUGCCCCCGUUG | 14166 | GTCTTCCGGTTGCCCCCGTTG |
| 8502 | GUCUUCUCUGUCUUUGAAUAA | 14167 | GTCTTCTCTGTCTTTGAATAA |
| 8503 | GUCUUCUGAUAGACGGGGUUG | 14168 | GTCTTCTGATAGACGGGGTTG |
| 8504 | GUCUUGGCACUGGAACUCGUU | 14169 | GTCTTGGCACTGGAACTCGTT |
| 8505 | GUCUUGGGGGAGCACGUCUCC | 14170 | GTCTTGGGGGAGCACGTCTCC |
| 8506 | GUCUUGGGGGGACAGCCUUGC | 14171 | GTCTTGGGGGGACAGCCTTGC |
| 8507 | GUCUUUGAAUAAAACAAGGCC | 14172 | GTCTTTGAATAAAACAAGGCC |
| 8508 | GUGAACUUGAUCGGGGCUGGU | 14173 | GTGAACTTGATCGGGGCTGGT |
| 8509 | GUGAAGAAGAGGUAGGCGAUG | 14174 | GTGAAGAAGAGGTAGGCGATG |
| 8510 | GUGAAGUUUGGAGUCAACCCA | 14175 | GTGAAGTTTGGAGTCAACCCA |
| 8511 | GUGAAUGAGUUUGGUCUCGGU | 14176 | GTGAATGAGTTTGGTCTCGGT |
| 8512 | GUGAAUGGCUUGGAGGCAUUU | 14177 | GTGAATGGCTTGGAGGCATTT |
| 8513 | GUGACACCCAUCUCCCAGAAG | 14178 | GTGACACCCATCTCCCAGAAG |
| 8514 | GUGACACUUGAACUUGUUGGG | 14179 | GTGACACTTGAACTTGTTGGG |
| 8515 | GUGACAGACAAGCACGUCUCC | 14180 | GTGACAGACAAGCACGTCTCC |
| 8516 | GUGACAGGAACCGCGGAGGAA | 14181 | GTGACAGGAACCGCGGAGGAA |
| 8517 | GUGACAGGGCAAAGGCUAACC | 14182 | GTGACAGGGCAAAGGCTAACC |
| 8518 | GUGACAUCUUCACGCGGGAGU | 14183 | GTGACATCTTCACGCGGGAGT |
| 8519 | GUGACCAGUGACUCAGGCUCU | 14184 | GTGACCAGTGACTCAGGCTCT |
| 8520 | GUGACUCAGGCUCUGCCUCUG | 14185 | GTGACTCAGGCTCTGCCTCTG |
| 8521 | GUGAGACAUUGUCACUAUCUC | 14186 | GTGAGACATTGTCACTATCTC |
| 8522 | GUGAGCACCGGGCAGGAGGCC | 14187 | GTGAGCACCGGGCAGGAGGCC |
| 8523 | GUGAGCCACUGCGCCCGGCCU | 14188 | GTGAGCCACTGCGCCCGGCCT |
| 8524 | GUGAGCCCAGGGGUGGCCCCA | 14189 | GTGAGCCCAGGGGTGGCCCCA |
| 8525 | GUGAGGCACCUGUGUCUGGCC | 14190 | GTGAGGCACCTGTGTCTGGCC |
| 8526 | GUGAGGCAGCUCCUCAUGUCC | 14191 | GTGAGGCAGCTCCTCATGTCC |
| 8527 | GUGAGGCGGUUGGCACUGAAA | 14192 | GTGAGGCGGTTGGCACTGAAA |
| 8528 | GUGAGGUUGUGGAAGAGAACC | 14193 | GTGAGGTTGTGGAAGAGAACC |
| 8529 | GUGAUAACAGUUUCAACCAAU | 14194 | GTGATAACAGTTTCAACCAAT |
| 8530 | GUGAUCCAGCUCAGCCUCCCA | 14195 | GTGATCCAGCTCAGCCTCCCA |
| 8531 | GUGAUGCAUUCGCCGCUGUGA | 14196 | GTGATGCATTCGCCGCTGTGA |
| 8532 | GUGAUGCCAUUGGGCCACUGA | 14197 | GTGATGCCATTGGGCCACTGA |
| 8533 | GUGCAAAAUAAUAACACAAAU | 14198 | GTGCAAAATAATAACACAAAT |
| 8534 | GUGCAAACAUAACAAAAAAUC | 14199 | GTGCAAACATAACAAAAAATC |
| 8535 | GUGCAAAUAAUCUUUGUACAU | 14200 | GTGCAAATAATCTTTGTACAT |
| 8536 | GUGCAACAGUAACACGGCGAU | 14201 | GTGCAACAGTAACACGGCGAT |
| 8537 | GUGCAAGGAGACCACGGGAAU | 14202 | GTGCAAGGAGACCACGGGAAT |
| 8538 | GUGCAGGCCACGUGUCCUCAC | 14203 | GTGCAGGCCACGTGTCCTCAC |
| 8539 | GUGCAGUGGCUCGAUCAUGGC | 14204 | GTGCAGTGGCTCGATCATGGC |
| 8540 | GUGCAUGUUUUAAACACAUAC | 14205 | GTGCATGTTTTAAACACATAC |
| 8541 | GUGCCACCACGGAUUCAGCCA | 14206 | GTGCCACCACGGATTCAGCCA |
| 8542 | GUGCCACCGUACCCAGCUGAU | 14207 | GTGCCACCGTACCCAGCTGAT |
| 8543 | GUGCCAUCUGCUGUUGUGUGU | 14208 | GTGCCATCTGCTGTTGTGTGT |
| 8544 | GUGCCCAGGACAGAGUCGGUC | 14209 | GTGCCCAGGACAGAGTCGGTC |
| 8545 | GUGCCCGCCACCACGCCCGGC | 14210 | GTGCCCGCCACCACGCCCGGC |
| 8546 | GUGCCGGUUGGUGAAGAAGAG | 14211 | GTGCCGGTTGGTGAAGAAGAG |
| 8547 | GUGCUCCGAAACCAGAAAGGC | 14212 | GTGCTCCGAAACCAGAAAGGC |
| 8548 | GUGCUGCAGAUCAUUCUCUGG | 14213 | GTGCTGCAGATCATTCTCTGG |
| 8549 | GUGCUGGGAUUACAGGCGUGA | 14214 | GTGCTGGGATTACAGGCGTGA |
| 8550 | GUGCUGUGUCCUUACGGCUGU | 14215 | GTGCTGTGTCCTTACGGCTGT |
| 8551 | GUGGAACUCGAAGGCCGAGCA | 14216 | GTGGAACTCGAAGGCCGAGCA |
| 8552 | GUGGAAGAGAACCAUAUCCUC | 14217 | GTGGAAGAGAACCATATCCTC |
| 8553 | GUGGAAGCACUAGGUGGGGG | 14218 | GTGGAAGCACTAGGTGGGCGG |
| 8554 | GUGGACCUCAUCCUCUGUGGU | 14219 | GTGGACCTCATCCTCTGTGGT |
| 8555 | GUGGAGAGAAACUCAAAACUU | 14220 | GTGGAGAGAAACTCAAAACTT |
| 8556 | GUGGAGCUGACCUUUAGCCUG | 14221 | GTGGAGCTGACCTTTAGCCTG |
| 8557 | GUGGAGCUGUUGCACUGGAAG | 14222 | GTGGAGCTGTTGCACTGGAAG |
| 8558 | GUGGAUCCAGUCCACAGCCAG | 14223 | GTGGATCCAGTCCACAGCCAG |
| 8559 | GUGGAUGAGGCAGGGCCAGGG | 14224 | GTGGATGAGGCAGGGCCAGGG |
| 8560 | GUGGAUGCACUCGCCACUUAG | 14225 | GTGGATGCACTCGCCACTTAG |
| 8561 | GUGGAUGUCUCCUGGGUGGCC | 14226 | GTGGATGTCTCCTGGGTGGCC |
| 8562 | GUGGCAAAUGUGGACCUCAUC | 14227 | GTGGCAAATGTGGACCTCATC |
| 8563 | GUGGCAGCGAAACUCGUCCUG | 14228 | GTGGCAGCGAAACTCGTCCTG |
| 8564 | GUGGCAUAAUCAUAGCUCACG | 14229 | GTGGCATAATCATAGCTCACG |
| 8565 | GUGGCAUCAUUUGGUGAAUGA | 14230 | GTGGCATCATTTGGTGAATGA |
| 8566 | GUGGCCACAGCGCAGUUUUCC | 14231 | GTGGCCACAGCGCAGTTTTCC |
| 8567 | GUGGCCACAGGACAGCCUUGC | 14232 | GTGGCCACAGGACAGCCTTGC |
| 8568 | GUGGCCACUGCAGCCUCAGCC | 14233 | GTGGCCACTGCAGCCTCAGCC |
| 8569 | GUGGCCCCAGGCAGCCGGGAG | 14234 | GTGGCCCCAGGCAGCCGGGAG |
| 8570 | GUGGCCGGGGGCCCCUCUGUG | 14235 | GTGGCCGGGGGCCCCTCTGTG |
| 8571 | GUGGCGAUGGGGAGGACAAUG | 14236 | GTGGCGATGGGGAGGACAATG |
| 8572 | GUGGCGCAAUCUCGGCUCACU | 14237 | GTGGCGCAATCTCGGCTCACT |
| 8573 | GUGGCUCGAUCAUGGCUCACU | 14238 | GTGGCTCGATCATGGCTCACT |
| 8574 | GUGGCUGAAGAUCUCGGCUGC | 14239 | GTGGCTGAAGATCTCGGCTGC |
| 8575 | GUGGGAACAGCCGCCGUUGUU | 14240 | GTGGGAACAGCCGCCGTTGTT |
| 8576 | GUGGGCCAGCCUCUUUUCAUC | 14241 | GTGGGCCAGCCTCTTTTCATC |
| 8577 | GUGGGCGGACAUAAAGACGAC | 14242 | GTGGGCGGACATAAAGACGAC |
| 8578 | GUGGGCUCUGUCAAGCUGGGU | 14243 | GTGGGCTCTGTCAAGCTGGGT |
| 8579 | GUGGGGACGGAGGUGUCGCCA | 14244 | GTGGGGACGGAGGTGTCGCCA |
| 8580 | GUGGGGCGAGGGCGCCCCCCA | 14245 | GTGGGGCGAGGGCGCCCCCCA |
| 8581 | GUGGGGGUCCAGCUGGAAGCC | 14246 | GTGGGGGTCCAGCTGGAAGCC |
| 8582 | GUGGGGGUUGAUCUGCGGGGC | 14247 | GTGGGGGTTGATCTGCGGGGC |
| 8583 | GUGGGUAGGGGUCGGGAGGAU | 14248 | GTGGGTAGGGGTCGGGAGGAT |
| 8584 | GUGGGUCAUGAUUCGGGGGUG | 14249 | GTGGGTCATGATTCGGGGGTG |
| 8585 | GUGGUCCUCUCACACCAGUUC | 14250 | GTGGTCCTCTCACACCAGTTC |
| 8586 | GUGGUCUUCUGAUAGACGGGG | 14251 | GTGGTCTTCTGATAGACGGGG |
| 8587 | GUGGUGAGCCCAGGGGUGGCC | 14252 | GTGGTGAGCCCAGGGGTGGCC |
| 8588 | GUGGUUGUGUGCUGUGUCCUU | 14253 | GTGGTTGTGTGCTGTGTCCTT |
| 8589 | GUGGUUUAAAAAGUGACACCC | 14254 | GTGGTTTAAAAAGTGACACCC |
| 8590 | GUGUACAACUCUGAACUGAGA | 14255 | GTGTACAACTCTGAACTGAGA |
| 8591 | GUGUACUCGCUCCGGUCCAGC | 14256 | GTGTACTCGCTCCGGTCCAGC |
| 8592 | GUGUAUAUAUAUAUAUAUAAG | 14257 | GTGTATATATATATATATAAG |
| 8593 | GUGUAUGUCGCGGAGGCGGCU | 14258 | GTGTATGTCGCGGAGGCGGCT |
| 8594 | GUGUCACAUUAACGCAGCCAA | 14259 | GTGTCACATTAACGCAGCCAA |
| 8595 | GUGUCACGGUGGAGAGAAACU | 14260 | GTGTCACGGTGGAGAGAAACT |
| 8596 | GUGUCAGACAGCCUUGCUCGU | 14261 | GTGTCAGACAGCCTTGCTCGT |
| 8597 | GUGUCAUAGGAAGAGACGCCG | 14262 | GTGTCATAGGAAGAGACGCCG |
| 8598 | GUGUCAUCCUGGUCCCCCCGG | 14263 | GTGTCATCCTGGTCCCCCCGG |
| 8599 | GUGUCCAGAGCGACCACGUUC | 14264 | GTGTCCAGAGCGACCACGTTC |
| 8600 | GUGUCCCGACCCGGAUCACGA | 14265 | GTGTCCCGACCCGGATCACGA |
| 8601 | GUGUCCUAGCUGGAAACCCUG | 14266 | GTGTCCTAGCTGGAAACCCTG |
| 8602 | GUGUCCUCACGGCCAAGGUAA | 14267 | GTGTCCTCACGGCCAAGGTAA |
| 8603 | GUGUCCUGACAGCCACACCUG | 14268 | GTGTCCTGACAGCCACACCTG |
| 8604 | GUGUCCUUACGGCUGUGGAGC | 14269 | GTGTCCTTACGGCTGTGGAGC |
| 8605 | GUGUCGCCACAGAGCACAGCG | 14270 | GTGTCGCCACAGAGCACAGCG |
| 8606 | GUGUCGGGAACAGGUCGGGUG | 14271 | GTGTCGGGAACAGGTCGGGTG |
| 8607 | GUGUCGGGAUCCUGACACUCA | 14272 | GTGTCGGGATCCTGACACTCA |
| 8608 | GUGUCUCAGGCACUUAAUAAA | 14273 | GTGTCTCAGGCACTTAATAAA |
| 8609 | GUGUCUGGCCAAAUUCAUUUU | 14274 | GTGTCTGGCCAAATTCATTTT |
| 8610 | GUGUGCUGUGUCCUUACGGCU | 14275 | GTGTGCTGTGTCCTTACGGCT |
| 8611 | GUGUGGGGGUCCAGCUGGAAG | 14276 | GTGTGGGGGTCCAGCTGGAAG |
| 8612 | GUGUGUAUAUAUAUAUAUAUA | 14277 | GTGTGTATATATATATATATA |
| 8613 | GUGUGUUGAAAGCCACUUGCU | 14278 | GTGTGTTGAAAGCCACTTGCT |
| 8614 | GUGUUAGCCAGGAUGGUCUCG | 14279 | GTGTTAGCCAGGATGGTCTCG |
| 8615 | GUGUUGAAAGCCACUUGCUUC | 14280 | GTGTTGAAAGCCACTTGCTTC |
| 8616 | GUGUUUUGUCCCUGGGGACAA | 14281 | GTGTTTTGTCCCTGGGGACAA |
| 8617 | GUUAGCCAGGAUGGUCUCGAU | 14282 | GTTAGCCAGGATGGTCTCGAT |
| 8618 | GUUCACACCAUUCUCCUGCCU | 14283 | GTTCACACCATTCTCCTGCCT |
| 8619 | GUUCACGCAGAGCUGGCUGCA | 14284 | GTTCACGCAGAGCTGGCTGCA |
| 8620 | GUUCACGCCACGUCAUCCUCC | 14285 | GTTCACGCCACGTCATCCTCC |
| 8621 | GUUCACUCCUCUUGGCUGGGU | 14286 | GTTCACTCCTCTTGGCTGGGT |
| 8622 | GUUCAUCUGACCAGUCCCGGC | 14287 | GTTCATCTGACCAGTCCCGGC |
| 8623 | GUUCAUUUAAGAAAUUUAAUU | 14288 | GTTCATTTAAGAAATTTAATT |
| 8624 | GUUCCCCAGUCAGUCCAGUAC | 14289 | GTTCCCCAGTCAGTCCAGTAC |
| 8625 | GUUCCCUGCACUGGGGGGGAC | 14290 | GTTCCCTGCACTGGGGGGGAC |
| 8626 | GUUCCUCAGGUUGGGGAUGAG | 14291 | GTTCCTCAGGTTGGGGATGAG |
| 8627 | GUUCGUGCAAAUAAUCUUUGU | 14292 | GTTCGTGCAAATAATCTTTGT |
| 8628 | GUUCUCCCUGAAUAACGUUUU | 14293 | GTTCTCCCTGAATAACGTTTT |
| 8629 | GUUCUCGAUAUUCACGAUCAC | 14294 | GTTCTCGATATTCACGATCAC |
| 8630 | GUUCUGCCUCCCAGAUGAAUA | 14295 | GTTCTGCCTCCCAGATGAATA |
| 8631 | GUUCUGGGCAGGGACGGGACU | 14296 | GTTCTGGGCAGGGACGGGACT |
| 8632 | GUUCUUAAGCCGCCAGUUCUU | 14297 | GTTCTTAAGCCGCCAGTTCTT |
| 8633 | GUUCUUCCAUAGAAGGAAGAC | 14298 | GTTCTTCCATAGAAGGAAGAC |
| 8634 | GUUGAAAGCCACUUGCUUCUC | 14299 | GTTGAAAGCCACTTGCTTCTC |
| 8635 | GUUGACACGGCCCCCACAGCU | 14300 | GTTGACACGGCCCCCACAGCT |
| 8636 | GUUGACAUCGAUGCUUGAGAU | 14301 | GTTGACATCGATGCTTGAGAT |
| 8637 | GUUGACAUCGGAACCUGUGAG | 14302 | GTTGACATCGGAACCTGTGAG |
| 8638 | GUUGACUCCUGAGUUUAGGGG | 14303 | GTTGACTCCTGAGTTTAGGGG |
| 8639 | GUUGAUCUGCGGGGCAGGGAG | 14304 | GTTGATCTGCGGGGCAGGGAG |
| 8640 | GUUGAUGAUAUCUGUCCAAAA | 14305 | GTTGATGATATCTGTCCAAAA |
| 8641 | GUUGAUGCUGUUGAUGUUCUU | 14306 | GTTGATGCTGTTGATGTTCTT |
| 8642 | GUUGAUGUUCUUAAGCCGCCA | 14307 | GTTGATGTTCTTAAGCCGCCA |
| 8643 | GUUGCACACAGUCCAGUUCGU | 14308 | GTTGCACACAGTCCAGTTCGT |
| 8644 | GUUGCACUGGAAGCUGGCGGG | 14309 | GTTGCACTGGAAGCTGGCGGG |
| 8645 | GUUGCAGACUUUGUCCAGGGU | 14310 | GTTGCAGACTTTGTCCAGGGT |
| 8646 | GUUGCCCAGGCUGGAAAGCAG | 14311 | GTTGCCCAGGCTGGAAAGCAG |
| 8647 | GUUGCCCAGGCUGGAGUGCAG | 14312 | GTTGCCCAGGCTGGAGTGCAG |
| 8648 | GUUGCCCCCGUUGACAUCGAU | 14313 | GTTGCCCCCGTTGACATCGAT |
| 8649 | GUUGCUCAGGCCUGGUCUCCA | 14314 | GTTGCTCAGGCCTGGTCTCCA |
| 8650 | GUUGCUGUGGAUCCAGUCCAC | 14315 | GTTGCTGTGGATCCAGTCCAC |
| 8651 | GUUGGCACUGAAAAUGGCUUC | 14316 | GTTGGCACTGAAAATGGCTTC |
| 8652 | GUUGGGGAUGAGGCUGGUGUA | 14317 | GTTGGGGATGAGGCTGGTGTA |
| 8653 | GUUGGGUCCCUCGCAGAGUGU | 14318 | GTTGGGTCCCTCGCAGAGTGT |
| 8654 | GUUGGUCCCGCACUCUUUGAU | 14319 | GTTGGTCCCGCACTCTTTGAT |
| 8655 | GUUGGUGAAGAAGAGGUAGGC | 14320 | GTTGGTGAAGAAGAGGTAGGC |
| 8656 | GUUGGUGCCAUCUGCUGUUGU | 14321 | GTTGGTGCCATCTGCTGTTGT |
| 8657 | GUUGUAUUUCUGAAAUAGGGU | 14322 | GTTGTATTTCTGAAATAGGGT |
| 8658 | GUUGUCAAAGUUGAUGCUGUU | 14323 | GTTGTCAAAGTTGATGCTGTT |
| 8659 | GUUGUCCAAGCAUUCGUUGGU | 14324 | GTTGTCCAAGCATTCGTTGGT |
| 8660 | GUUGUCGCAGGCCCACAGCUG | 14325 | GTTGTCGCAGGCCCACAGCTG |
| 8661 | GUUGUCGCAGUCCACUUGGCC | 14326 | GTTGTCGCAGTCCACTTGGCC |
| 8662 | GUUGUGGAAGAGAACCAUAUC | 14327 | GTTGTGGAAGAGAACCATATC |
| 8663 | GUUGUGGCAAAUGUGGACCUC | 14328 | GTTGTGGCAAATGTGGACCTC |
| 8664 | GUUGUGUGCUGUGUCCUUACG | 14329 | GTTGTGTGCTGTGTCCTTACG |
| 8665 | GUUGUGUGUUGAAAGCCACUU | 14330 | GTTGTGTGTTGAAAGCCACTT |
| 8666 | GUUGUUGUCCAAGCAUUCGUU | 14331 | GTTGTTGTCCAAGCATTCGTT |
| 8667 | GUUUAAAAAGUGACACCCAUC | 14332 | GTTTAAAAAGTGACACCCATC |
| 8668 | GUUUAGGGGUUUGGCUUAGAG | 14333 | GTTTAGGGGTTTGGCTTAGAG |
| 8669 | GUUUCAACCAAUAUACAAAGU | 14334 | GTTTCAACCAATATACAAAGT |
| 8670 | GUUUCAACCUCGUAAGGAACC | 14335 | GTTTCAACCTCGTAAGGAACC |
| 8671 | GUUUCACGUGUUAGCCAGGAU | 14336 | GTTTCACGTGTTAGCCAGGAT |
| 8672 | GUUUCCAUCAGAGCACUGGAA | 14337 | GTTTCCATCAGAGCACTGGAA |
| 8673 | GUUUCUCUUAUCCUUCACGAG | 14338 | GTTTCTCTTATCCTTCACGAG |
| 8674 | GUUUCUUUCGCAUCUGUCGCC | 14339 | GTTTCTTTCGCATCTGTCGCC |
| 8675 | GUUUGAGGAUUGUGUCACGGU | 14340 | GTTTGAGGATTGTGTCACGGT |
| 8676 | GUUUGCAAAAAUAAAUAGAUU | 14341 | GTTTGCAAAAATAAATAGATT |
| 8677 | GUUUGGAGUCAACCCAGUAGA | 14342 | GTTTGGAGTCAACCCAGTAGA |
| 8678 | GUUUGGCUUAGAGAUUGGUGG | 14343 | GTTTGGCTTAGAGATTGGTGG |
| 8679 | GUUUGGUCUCGGUGCCACCAC | 14344 | GTTTGGTCTCGGTGCCACCAC |
| 8680 | GUUUUAAACACAUACCCAUCA | 14345 | GTTTTAAACACATACCCATCA |
| 8681 | GUUUUCAGCCAACAAGUUGAC | 14346 | GTTTTCAGCCAACAAGTTGAC |
| 8682 | GUUUUCAGUCACCAGCGAGUA | 14347 | GTTTTCAGTCACCAGCGAGTA |
| 8683 | GUUUUCCUCGUCAGAUUUGUC | 14348 | GTTTTCCTCGTCAGATTTGTC |
| 8684 | GUUUUCCUCUUCACGCCCUUG | 14349 | GTTTTCCTCTTCACGCCCTTG |
| 8685 | GUUUUGUCCCUGGGGACAAUU | 14350 | GTTTTGTCCCTGGGGACAATT |
| 8686 | GUUUUUGUAGAGAUGGGAUCU | 14351 | GTTTTTGTAGAGATGGGATCT |
| 8687 | GUUUUUUUUUGUUUUUUUUUU | 14352 | GTTTTTTTTTGTTTTTTTTTT |
| 8688 | GUUUUUUUUUUUUGAGACGGA | 14353 | GTTTTTTTTTTTTGAGACGGA |
| 8689 | UAAAAAGUGACACCCAUCUCC | 14354 | TAAAAAGTGACACCCATCTCC |
| 8690 | UAAAACAAAGCUCUGGCAGGC | 14355 | TAAAACAAAGCTCTGGCAGGC |
| 8691 | UAAAACAAGGCCGGCGAGGUC | 14356 | TAAAACAAGGCCGGCGAGGTC |
| 8692 | UAAACACAUACCCAUCAACGA | 14357 | TAAACACATACCCATCAACGA |
| 8693 | UAAACACGUUGACUCCUGAGU | 14358 | TAAACACGTTGACTCCTGAGT |
| 8694 | UAAACUUGGGCGAGUGGGGGU | 14359 | TAAACTTGGGCGAGTGGGGGT |
| 8695 | UAAAGAAGAGGCAUUUCAGAG | 14360 | TAAAGAAGAGGCATTTCAGAG |
| 8696 | UAAAGACCCCUACAGCGCUGC | 14361 | TAAAGACCCCTACAGCGCTGC |
| 8697 | UAAAGACGACAAUGGCAGUUC | 14362 | TAAAGACGACAATGGCAGTTC |
| 8698 | UAAAUAGAUUUUAUAUAUAUG | 14363 | TAAATAGATTTTATATATATG |
| 8699 | UAAAUAUAUAAAACAAAGCUC | 14364 | TAAATATATAAAACAAAGCTC |
| 8700 | UAAAUAUUAAGGGUGACCAGU | 14365 | TAAATATTAAGGGTGACCAGT |
| 8701 | UAACAAAAAAUCCCCACUAUC | 14366 | TAACAAAAAATCCCCACTATC |
| 8702 | UAACACAAAUGCCAAAUGUAC | 14367 | TAACACAAATGCCAAATGTAC |
| 8703 | UAACACGGCGAUUUCAGAAAG | 14368 | TAACACGGCGATTTCAGAAAG |
| 8704 | UAACAGUUUCAACCAAUAUAC | 14369 | TAACAGTTTCAACCAATATAC |
| 8705 | UAACCCCCUACACAGGGCCCC | 14370 | TAACCCCCTACACAGGGCCCC |
| 8706 | UAACCGGGUGUCUCAGGCACU | 14371 | TAACCGGGTGTCTCAGGCACT |
| 8707 | UAACCUGGCUGUCUAGCAAGG | 14372 | TAACCTGGCTGTCTAGCAAGG |
| 8708 | UAACGCAGCCAACUUCAUCGC | 14373 | TAACGCAGCCAACTTCATCGC |
| 8709 | UAACGGAGGGCGACUCGUGUA | 14374 | TAACGGAGGGCGACTCGTGTA |
| 8710 | UAACGUUUUCCUCUUCACGCC | 14375 | TAACGTTTTCCTCTTCACGCC |
| 8711 | UAACUUUUUGGUUUUUGUAGA | 14376 | TAACTTTTTGGTTTTTGTAGA |
| 8712 | UAAGAAAUUUAAUUGAUCACA | 14377 | TAAGAAATTTAATTGATCACA |
| 8713 | UAAGACAUUCAGGCUACAAAA | 14378 | TAAGACATTCAGGCTACAAAA |
| 8714 | UAAGAGAUGGGGUCUUACUAU | 14379 | TAAGAGATGGGGTCTTACTAT |
| 8715 | UAAGCCGCCAGUUCUUCCAUA | 14380 | TAAGCCGCCAGTTCTTCCATA |
| 8716 | UAAGGAACCGAGGCCUUGAGG | 14381 | TAAGGAACCGAGGCCTTGAGG |
| 8717 | UAAGGAAGGUACAAACCCACC | 14382 | TAAGGAAGGTACAAACCCACC |
| 8718 | UAAGGGUGACCAGUGACUCAG | 14383 | TAAGGGTGACCAGTGACTCAG |
| 8719 | UAAGGUCAUUGCAGACGUGGG | 14384 | TAAGGTCATTGCAGACGTGGG |
| 8720 | UAAGUGAUAACAGUUUCAACC | 14385 | TAAGTGATAACAGTTTCAACC |
| 8721 | UAAUAAAUAUUAAGGGUGACC | 14386 | TAATAAATATTAAGGGTGACC |
| 8722 | UAAUAACACAAAUGCCAAAUG | 14387 | TAATAACACAAATGCCAAATG |
| 8723 | UAAUCAUAGCUCACGACAGCC | 14388 | TAATCATAGCTCACGACAGCC |
| 8724 | UAAUCUUUGUACAUAAAGAAG | 14389 | TAATCTTTGTACATAAAGAAG |
| 8725 | UAAUUGAUCACAGUAAGACAU | 14390 | TAATTGATCACAGTAAGACAT |
| 8726 | UAAUUUUUUGUAUUUUUAGUA | 14391 | TAATTTTTTGTATTTTTAGTA |
| 8727 | UACAAAAACCUUCUAUACAGU | 14392 | TACAAAAACCTTCTATACAGT |
| 8728 | UACAAACCCACCUCGAAAGAC | 14393 | TACAAACCCACCTCGAAAGAC |
| 8729 | UACAAAGUGCAAACAUAACAA | 14394 | TACAAAGTGCAAACATAACAA |
| 8730 | UACAACGGGGACAUCAUUCUC | 14395 | TACAACGGGGACATCATTCTC |
| 8731 | UACAACUCUGAACUGAGAAAG | 14396 | TACAACTCTGAACTGAGAAAG |
| 8732 | UACACAGGGCCCCGAGAAUAG | 14397 | TACACAGGGCCCCGAGAATAG |
| 8733 | UACACAGUGUACAACUCUGAA | 14398 | TACACAGTGTACAACTCTGAA |
| 8734 | UACAGCAACCAGGGUUUGCAA | 14399 | TACAGCAACCAGGGTTTGCAA |
| 8735 | UACAGCGCUGCGGCCACUCAU | 14400 | TACAGCGCTGCGGCCACTCAT |
| 8736 | UACAGGCGUGAGCCACUGCGC | 14401 | TACAGGCGTGAGCCACTGCGC |
| 8737 | UACAGGCGUGAGGCACCUGUG | 14402 | TACAGGCGTGAGGCACCTGTG |
| 8738 | UACAGGGACGCAUUUACGUGC | 14403 | TACAGGGACGCATTTACGTGC |
| 8739 | UACAGGUGCCCGCCACCACGC | 14404 | TACAGGTGCCCGCCACCACGC |
| 8740 | UACAGUGGUUUAAAAAGUGAC | 14405 | TACAGTGGTTTAAAAAGTGAC |
| 8741 | UACAUAAAGAAGAGGCAUUUC | 14406 | TACATAAAGAAGAGGCATTTC |
| 8742 | UACAUACAACGGGGACAUCAU | 14407 | TACATACAACGGGGACATCAT |
| 8743 | UACAUGAAGCCAUGAACAGGA | 14408 | TACATGAAGCCATGAACAGGA |
| 8744 | UACCCAGCUGAUUUUUAACUU | 14409 | TACCCAGCTGATTTTTAACTT |
| 8745 | UACCCAUCAACGACAAGAUUG | 14410 | TACCCATCAACGACAAGATTG |
| 8746 | UACGGCUGUGGAGCUGACCUU | 14411 | TACGGCTGTGGAGCTGACCTT |
| 8747 | UACGUGCUCCGAAACCAGAAA | 14412 | TACGTGCTCCGAAACCAGAAA |
| 8748 | UACUAUGUUGCCCAGGCUGGA | 14413 | TACTATGTTGCCCAGGCTGGA |
| 8749 | UACUCGCUCCGGUCCAGCGUC | 14414 | TACTCGCTCCGGTCCAGCGTC |
| 8750 | UACUGGCAGCCGCCAUUGCUC | 14415 | TACTGGCAGCCGCCATTGCTC |
| 8751 | UACUGGGCUUCUUCUCAUUUC | 14416 | TACTGGGCTTCTTCTCATTTC |
| 8752 | UACUGUCCCCUUGGAACACGU | 14417 | TACTGTCCCCTTGGAACACGT |
| 8753 | UACUUUGUCCUCAAAGACGGC | 14418 | TACTTTGTCCTCAAAGACGGC |
| 8754 | UAGAAGAGGUAAACACGUUGA | 14419 | TAGAAGAGGTAAACACGTTGA |
| 8755 | UAGAAGGAAGACCCCCAGGCA | 14420 | TAGAAGGAAGACCCCCAGGCA |
| 8756 | UAGAAGUGGAAGCACUAGGUG | 14421 | TAGAAGTGGAAGCACTAGGTG |
| 8757 | UAGACGGGGUUGUCAAAGUUG | 14422 | TAGACGGGGTTGTCAAAGTTG |
| 8758 | UAGAGACGGGGUUUCACGUGU | 14423 | TAGAGACGGGGTTTCACGTGT |
| 8759 | UAGAGAUGGGAUCUCGCUUUG | 14424 | TAGAGATGGGATCTCGCTTTG |
| 8760 | UAGAGAUUGGUGGAUGAGGCA | 14425 | TAGAGATTGGTGGATGAGGCA |
| 8761 | UAGAGGCGGCCACUGAGGAGA | 14426 | TAGAGGCGGCCACTGAGGAGA |
| 8762 | UAGAUGUCCACACCAUUCAGG | 14427 | TAGATGTCCACACCATTCAGG |
| 8763 | UAGAUGUUGCUGUGGAUCCAG | 14428 | TAGATGTTGCTGTGGATCCAG |
| 8764 | UAGAUUCUAUUGCUGGCCACC | 14429 | TAGATTCTATTGCTGGCCACC |
| 8765 | UAGAUUUUAUAUAUAUGUGUG | 14430 | TAGATTTTATATATATGTGTG |
| 8766 | UAGCAAGGCUUGCAUAGAAGA | 14431 | TAGCAAGGCTTGCATAGAAGA |
| 8767 | UAGCCACCCUCCAGGUUCACG | 14432 | TAGCCACCCTCCAGGTTCACG |
| 8768 | UAGCCAGGAUGGUCUCGAUCU | 14433 | TAGCCAGGATGGTCTCGATCT |
| 8769 | UAGCCAUGUUGCAGACUUUGU | 14434 | TAGCCATGTTGCAGACTTTGT |
| 8770 | UAGCCGAUCUUAAGGUCAUUG | 14435 | TAGCCGATCTTAAGGTCATTG |
| 8771 | UAGCCGUCCUGGUUGUGGCAA | 14436 | TAGCCGTCCTGGTTGTGGCAA |
| 8772 | UAGCCUGACGGUGGAUGUCUC | 14437 | TAGCCTGACGGTGGATGTCTC |
| 8773 | UAGCUCACGACAGCCUCAAAU | 14438 | TAGCTCACGACAGCCTCAAAT |
| 8774 | UAGCUGGAAACCCUGGCUUCC | 14439 | TAGCTGGAAACCCTGGCTTCC |
| 8775 | UAGCUGGGACUACAGGUGCCC | 14440 | TAGCTGGGACTACAGGTGCCC |
| 8776 | UAGCUGGGAUCACAGGCACGU | 14441 | TAGCTGGGATCACAGGCACGT |
| 8777 | UAGCUGUAGCCGUCCUGGUUG | 14442 | TAGCTGTAGCCGTCCTGGTTG |
| 8778 | UAGGAAGAGACGCCGUGGGCU | 14443 | TAGGAAGAGACGCCGTGGGCT |
| 8779 | UAGGAGAUGCAUUUCCCGUCU | 14444 | TAGGAGATGCATTTCCCGTCT |
| 8780 | UAGGCAGUGGAACUCGAAGGC | 14445 | TAGGCAGTGGAACTCGAAGGC |
| 8781 | UAGGCGAUGGAGCCCACAGCC | 14446 | TAGGCGATGGAGCCCACAGCC |
| 8782 | UAGGGAGAGGCACGCUCUGUC | 14447 | TAGGGAGAGGCACGCTCTGTC |
| 8783 | UAGGGGUCGGGAGGAUGGGCA | 14448 | TAGGGGTCGGGAGGATGGGCA |
| 8784 | UAGGGGUUUGGCUUAGAGAUU | 14449 | TAGGGGTTTGGCTTAGAGATT |
| 8785 | UAGGGUCUUCAUCUGUUGCCC | 14450 | TAGGGTCTTCATCTGTTGCCC |
| 8786 | UAGGGUGAUGCCAUUGGGCCA | 14451 | TAGGGTGATGCCATTGGGCCA |
| 8787 | UAGGUGGGCGGACAUAAAGAC | 14452 | TAGGTGGGCGGACATAAAGAC |
| 8788 | UAGGUUUUCAGCCAACAAGUU | 14453 | TAGGTTTTCAGCCAACAAGTT |
| 8789 | UAGUAGAGACGGGGUUUCACG | 14454 | TAGTAGAGACGGGGTTTCACG |
| 8790 | UAGUCACUGAACAAAUACAGC | 14455 | TAGTCACTGAACAAATACAGC |
| 8791 | UAGUUGUAUUUCUGAAAUAGG | 14456 | TAGTTGTATTTCTGAAATAGG |
| 8792 | UAUAAAACAAAGCUCUGGCAG | 14457 | TATAAAACAAAGCTCTGGCAG |
| 8793 | UAUAAGUGAUAACAGUUUCAA | 14458 | TATAAGTGATAACAGTTTCAA |
| 8794 | UAUACAAAGUGCAAACAUAAC | 14459 | TATACAAAGTGCAAACATAAC |
| 8795 | UAUACAGUGGUUUAAAAAGUG | 14460 | TATACAGTGGTTTAAAAAGTG |
| 8796 | UAUAGUUGUAUUUCUGAAAUA | 14461 | TATAGTTGTATTTCTGAAATA |
| 8797 | UAUAUAAAACAAAGCUCUGGC | 14462 | TATATAAAACAAAGCTCTGGC |
| 8798 | UAUAUAAGUGAUAACAGUUUC | 14463 | TATATAAGTGATAACAGTTTC |
| 8799 | UAUAUAUAAGUGAUAACAGUU | 14464 | TATATATAAGTGATAACAGTT |
| 8800 | UAUAUAUAUAAGUGAUAACAG | 14465 | TATATATATAAGTGATAACAG |
| 8801 | UAUAUAUAUAUAAGUGAUAAC | 14466 | TATATATATATAAGTGATAAC |
| 8802 | UAUAUAUAUAUAUAAGUGAUA | 14467 | TATATATATATATAAGTGATA |
| 8803 | UAUAUAUAUAUAUAUAAGUGA | 14468 | TATATATATATATATAAGTGA |
| 8804 | UAUAUAUAUGUGUGUAUAUAU | 14469 | TATATATATGTGTGTATATAT |
| 8805 | UAUAUAUGUGUGUAUAUAUAU | 14470 | TATATATGTGTGTATATATAT |
| 8806 | UAUAUGUGUGUAUAUAUAUAU | 14471 | TATATGTGTGTATATATATAT |
| 8807 | UAUCACGGUUCCCUGCACUGG | 14472 | TATCACGGTTCCCTGCACTGG |
| 8808 | UAUCCGCAACAGAGACAGUGC | 14473 | TATCCGCAACAGAGACAGTGC |
| 8809 | UAUCCUCUGGGGACAGUAGGU | 14474 | TATCCTCTGGGGACAGTAGGT |
| 8810 | UAUCCUUCACGAGGAAAGGAA | 14475 | TATCCTTCACGAGGAAAGGAA |
| 8811 | UAUCGCACUCUUUGAUGGGUU | 14476 | TATCGCACTCTTTGATGGGTT |
| 8812 | UAUCUCCACCGUGGUGAGCCC | 14477 | TATCTCCACCGTGGTGAGCCC |
| 8813 | UAUCUGUACAGGGACGCAUUU | 14478 | TATCTGTACAGGGACGCATTT |
| 8814 | UAUCUGUCCAAAAUACUUUGU | 14479 | TATCTGTCCAAAATACTTTGT |
| 8815 | UAUCUUCGCAUCUUCGCUGGG | 14480 | TATCTTCGCATCTTCGCTGGG |
| 8816 | UAUGUCGCGGAGGCGGCUCCG | 14481 | TATGTCGCGGAGGCGGCTCCG |
| 8817 | UAUGUGUGUAUAUAUAUAUAU | 14482 | TATGTGTGTATATATATATAT |
| 8818 | UAUGUUGCCCAGGCUGGAAAG | 14483 | TATGTTGCCCAGGCTGGAAAG |
| 8819 | UAUUAAGGGUGACCAGUGACU | 14484 | TATTAAGGGTGACCAGTGACT |
| 8820 | UAUUCACGAUCACGGCUUUGA | 14485 | TATTCACGATCACGGCTTTGA |
| 8821 | UAUUCCCGGUCACACUGCCGG | 14486 | TATTCCCGGTCACACTGCCGG |
| 8822 | UAUUGCUGGCCACCUCCGUGU | 14487 | TATTGCTGGCCACCTCCGTGT |
| 8823 | UAUUUAUUUUUUUAUAGUUGU | 14488 | TATTTATTTTTTTATAGTTGT |
| 8824 | UAUUUCUGAAAUAGGGUCUUC | 14489 | TATTTCTGAAATAGGGTCTTC |
| 8825 | UAUUUUUAGUAGAGACGGGGU | 14490 | TATTTTTAGTAGAGACGGGGT |
| 8826 | UAUUUUUUUAUAGUUGUAUUU | 14491 | TATTTTTTTATAGTTGTATTT |
| 8827 | UCAAAACUUCCUGGAGAGAAA | 14492 | TCAAAACTTCCTGGAGAGAAA |
| 8828 | UCAAACCAUCCUCCCACCUCA | 14493 | TCAAACCATCCTCCCACCTCA |
| 8829 | UCAAACGAUCCAGACUGGAGG | 14494 | TCAAACGATCCAGACTGGAGG |
| 8830 | UCAAAGACGGCCAAGGAGAAG | 14495 | TCAAAGACGGCCAAGGAGAAG |
| 8831 | UCAAAGUUGAUGCUGUUGAUG | 14496 | TCAAAGTTGATGCTGTTGATG |
| 8832 | UCAAAUGCCUGAGCUCAAACC | 14497 | TCAAATGCCTGAGCTCAAACC |
| 8833 | UCAACCAAUAUACAAAGUGCA | 14498 | TCAACCAATATACAAAGTGCA |
| 8834 | UCAACCCAGUAGAGGCGGCCA | 14499 | TCAACCCAGTAGAGGCGGCCA |
| 8835 | UCAACCUCGUAAGGAACCGAG | 14500 | TCAACCTCGTAAGGAACCGAG |
| 8836 | UCAACCUGCCCCUCUCUGUCC | 14501 | TCAACCTGCCCCTCTCTGTCC |
| 8837 | UCAACGACAAGAUUGGGGAAG | 14502 | TCAACGACAAGATTGGGGAAG |
| 8838 | UCAAGCUGGGUGCUGCAGAUC | 14503 | TCAAGCTGGGTGCTGCAGATC |
| 8839 | UCAAGUGAUCCAGCUCAGCCU | 14504 | TCAAGTGATCCAGCTCAGCCT |
| 8840 | UCACACAGCUGCCCCCGAGAC | 14505 | TCACACAGCTGCCCCCGAGAC |
| 8841 | UCACACCAGUUCACUCCUCUU | 14506 | TCACACCAGTTCACTCCTCTT |
| 8842 | UCACACCAUUCUCCUGCCUCA | 14507 | TCACACCATTCTCCTGCCTCA |
| 8843 | UCACACUGCCGGCUGCCAUGG | 14508 | TCACACTGCCGGCTGCCATGG |
| 8844 | UCACACUGGCACUUGUAGCCA | 14509 | TCACACTGGCACTTGTAGCCA |
| 8845 | UCACAGACGAACUGCCGAGAG | 14510 | TCACAGACGAACTGCCGAGAG |
| 8846 | UCACAGCGCCAGCUGGAGUGG | 14511 | TCACAGCGCCAGCTGGAGTGG |
| 8847 | UCACAGGCACGUGCCACCGUA | 14512 | TCACAGGCACGTGCCACCGTA |
| 8848 | UCACAGUAAGACAUUCAGGCU | 14513 | TCACAGTAAGACATTCAGGCT |
| 8849 | UCACAUUAACGCAGCCAACUU | 14514 | TCACATTAACGCAGCCAACTT |
| 8850 | UCACCAGCGAGUAGAUGUCCA | 14515 | TCACCAGCGAGTAGATGTCCA |
| 8851 | UCACCGUGCAUGUUUUAAACA | 14516 | TCACCGTGCATGTTTTAAACA |
| 8852 | UCACGACAGCCUCAAAUGCCU | 14517 | TCACGACAGCCTCAAATGCCT |
| 8853 | UCACGACCUGCUGUGUCCUAG | 14518 | TCACGACCTGCTGTGTCCTAG |
| 8854 | UCACGAGGAAAGGAAGAAACC | 14519 | TCACGAGGAAAGGAAGAAACC |
| 8855 | UCACGAUCACGGCUUUGAAGU | 14520 | TCACGATCACGGCTTTGAAGT |
| 8856 | UCACGCAGAGCUGGCUGCAGG | 14521 | TCACGCAGAGCTGGCTGCAGG |
| 8857 | UCACGCCACGUCAUCCUCCAG | 14522 | TCACGCCACGTCATCCTCCAG |
| 8858 | UCACGCCCUUGGUAUCCGCAA | 14523 | TCACGCCCTTGGTATCCGCAA |
| 8859 | UCACGCGGGAGUCUGACGGAG | 14524 | TCACGCGGGAGTCTGACGGAG |
| 8860 | UCACGCUACUGGGCUUCUUCU | 14525 | TCACGCTACTGGGCTTCTTCT |
| 8861 | UCACGGCCAAGGUAACCGGGU | 14526 | TCACGGCCAAGGTAACCGGGT |
| 8862 | UCACGGCUUUGAAGUCCCGGU | 14527 | TCACGGCTTTGAAGTCCCGGT |
| 8863 | UCACGGUGGAGAGAAACUCAA | 14528 | TCACGGTGGAGAGAAACTCAA |
| 8864 | UCACGGUUCCCUGCACUGGGG | 14529 | TCACGGTTCCCTGCACTGGGG |
| 8865 | UCACGUGUUAGCCAGGAUGGU | 14530 | TCACGTGTTAGCCAGGATGGT |
| 8866 | UCACUAUCUCCACCGUGGUGA | 14531 | TCACTATCTCCACCGTGGTGA |
| 8867 | UCACUCCUCUUGGCUGGGUGA | 14532 | TCACTCCTCTTGGCTGGGTGA |
| 8868 | UCACUGAACAAAUACAGCAAC | 14533 | TCACTGAACAAATACAGCAAC |
| 8869 | UCACUGCAACCUCCACCUCCU | 14534 | TCACTGCAACCTCCACCTCCT |
| 8870 | UCACUGCAAGCUCCGCUUCCC | 14535 | TCACTGCAAGCTCCGCTTCCC |
| 8871 | UCACUGUCUCUCCGGACAUCA | 14536 | TCACTGTCTCTCCGGACATCA |
| 8872 | UCAGAAAGAACCUGAAGUCCC | 14537 | TCAGAAAGAACCTGAAGTCCC |
| 8873 | UCAGACAGCCUUGCUCGUCUG | 14538 | TCAGACAGCCTTGCTCGTCTG |
| 8874 | UCAGACCAGUAGAUUCUAUUG | 14539 | TCAGACCAGTAGATTCTATTG |
| 8875 | UCAGAGCACUGGAAUUCGUCA | 14540 | TCAGAGCACTGGAATTCGTCA |
| 8876 | UCAGAGCCAUCCUGGCACUCA | 14541 | TCAGAGCCATCCTGGCACTCA |
| 8877 | UCAGAGGCAAUAACCCCCUAC | 14542 | TCAGAGGCAATAACCCCCTAC |
| 8878 | UCAGAUUUGUCCUUGCAGUCG | 14543 | TCAGATTTGTCCTTGCAGTCG |
| 8879 | UCAGCCAACAAGUUGACAUCG | 14544 | TCAGCCAACAAGTTGACATCG |
| 8880 | UCAGCCAGAUCAUUUCCGACG | 14545 | TCAGCCAGATCATTTCCGACG |
| 8881 | UCAGCCUCCCAAAGUGCUGGG | 14546 | TCAGCCTCCCAAAGTGCTGGG |
| 8882 | UCAGCCUCCCAAGUAGCUGGG | 14547 | TCAGCCTCCCAAGTAGCTGGG |
| 8883 | UCAGCCUCCCGAGUAGCUGGG | 14548 | TCAGCCTCCCGAGTAGCTGGG |
| 8884 | UCAGCCUCGCCAUGCCUGGCC | 14549 | TCAGCCTCGCCATGCCTGGCC |
| 8885 | UCAGCCUCUCUUGGCUGGGUG | 14550 | TCAGCCTCTCTTGGCTGGGTG |
| 8886 | UCAGCCUCUGUGAGGCAGCUC | 14551 | TCAGCCTCTGTGAGGCAGCTC |
| 8887 | UCAGCGCUGCCAUCGCAGACC | 14552 | TCAGCGCTGCCATCGCAGACC |
| 8888 | UCAGGAAGGGUUCUGGGCAGG | 14553 | TCAGGAAGGGTTCTGGGCAGG |
| 8889 | UCAGGCACUUAAUAAAUAUUA | 14554 | TCAGGCACTTAATAAATATTA |
| 8890 | UCAGGCCCCCUUUCUUGAUCU | 14555 | TCAGGCCCCCTTTCTTGATCT |
| 8891 | UCAGGCCUGGUCUCCAACUCC | 14556 | TCAGGCCTGGTCTCCAACTCC |
| 8892 | UCAGGCGAUCCUCCUGCCUCA | 14557 | TCAGGCGATCCTCCTGCCTCA |
| 8893 | UCAGGCUACAAAAACCUUCUA | 14558 | TCAGGCTACAAAAACCTTCTA |
| 8894 | UCAGGCUCUGCCUCUGGGAAG | 14559 | TCAGGCTCTGCCTCTGGGAAG |
| 8895 | UCAGGGCGACAGGUGGCCACA | 14560 | TCAGGGCGACAGGTGGCCACA |
| 8896 | UCAGGGUGGUCCUCUCACACC | 14561 | TCAGGGTGGTCCTCTCACACC |
| 8897 | UCAGGUUGGGGAUGAGGCUGG | 14562 | TCAGGTTGGGGATGAGGCTGG |
| 8898 | UCAGUCACCAGCGAGUAGAUG | 14563 | TCAGTCACCAGCGAGTAGATG |
| 8899 | UCAGUCCAGUACAUGAAGCCA | 14564 | TCAGTCCAGTACATGAAGCCA |
| 8900 | UCAGUGCAACAGUAACACGGC | 14565 | TCAGTGCAACAGTAACACGGC |
| 8901 | UCAUACAUACAACGGGGACAU | 14566 | TCATACATACAACGGGGACAT |
| 8902 | UCAUAGCUCACGACAGCCUCA | 14567 | TCATAGCTCACGACAGCCTCA |
| 8903 | UCAUAGGAAGAGACGCCGUGG | 14568 | TCATAGGAAGAGACGCCGTGG |
| 8904 | UCAUAUUCCCGGUCACACUGC | 14569 | TCATATTCCCGGTCACACTGC |
| 8905 | UCAUCAGAGCCAUCCUGGCAC | 14570 | TCATCAGAGCCATCCTGGCAC |
| 8906 | UCAUCCGAGCCAUCUUCGCAG | 14571 | TCATCCGAGCCATCTTCGCAG |
| 8907 | UCAUCCUCCAAGAUGGUCUUC | 14572 | TCATCCTCCAAGATGGTCTTC |
| 8908 | UCAUCCUCCAGACUGACCAUC | 14573 | TCATCCTCCAGACTGACCATC |
| 8909 | UCAUCCUCUGUGGUCUUCUGA | 14574 | TCATCCTCTGTGGTCTTCTGA |
| 8910 | UCAUCCUGGUCCCCCCGGGCC | 14575 | TCATCCTGGTCCCCCCGGGCC |
| 8911 | UCAUCGAUAUCGCACUCUUUG | 14576 | TCATCGATATCGCACTCTTTG |
| 8912 | UCAUCGAUAUCUUCGCAUCUU | 14577 | TCATCGATATCTTCGCATCTT |
| 8913 | UCAUCGCUCAUGUCCUUGCAG | 14578 | TCATCGCTCATGTCCTTGCAG |
| 8914 | UCAUCUGACCAGUCCCGGCAG | 14579 | TCATCTGACCAGTCCCGGCAG |
| 8915 | UCAUCUGUUGCCCAGGCUGGA | 14580 | TCATCTGTTGCCCAGGCTGGA |
| 8916 | UCAUCUUCCAUGUUUGAGGAU | 14581 | TCATCTTCCATGTTTGAGGAT |
| 8917 | UCAUCUUCCUGACCUCGUGCC | 14582 | TCATCTTCCTGACCTCGTGCC |
| 8918 | UCAUGAUCCACCCGCCUCGGC | 14583 | TCATGATCCACCCGCCTCGGC |
| 8919 | UCAUGAUUCGGGGGUGACAGG | 14584 | TCATGATTCGGGGGTGACAGG |
| 8920 | UCAUGGCUCACUGCAACCUCC | 14585 | TCATGGCTCACTGCAACCTCC |
| 8921 | UCAUGUCCCUGGCCAGCAGCA | 14586 | TCATGTCCCTGGCCAGCAGCA |
| 8922 | UCAUGUCCUUGCAGUCAUAUU | 14587 | TCATGTCCTTGCAGTCATATT |
| 8923 | UCAUUCUCCCAAAAAGCGUUG | 14588 | TCATTCTCCCAAAAAGCGTTG |
| 8924 | UCAUUCUCUGGGACAGGUCAG | 14589 | TCATTCTCTGGGACAGGTCAG |
| 8925 | UCAUUGACACGGGCUUUCCCU | 14590 | TCATTGACACGGGCTTTCCCT |
| 8926 | UCAUUGCAGACGUGGGAACAG | 14591 | TCATTGCAGACGTGGGAACAG |
| 8927 | UCAUUUAAGAAAUUUAAUUGA | 14592 | TCATTTAAGAAATTTAATTGA |
| 8928 | UCAUUUCCGACGCCAUGAAUC | 14593 | TCATTTCCGACGCCATGAATC |
| 8929 | UCAUUUCCUCUGCCAGCAACG | 14594 | TCATTTCCTCTGCCAGCAACG |
| 8930 | UCAUUUGGUGAAUGAGUUUGG | 14595 | TCATTTGGTGAATGAGTTTGG |
| 8931 | UCAUUUUUUAAGAGAUGGGGU | 14596 | TCATTTTTTAAGAGATGGGGT |
| 8932 | UCCAAAAUACUUUGUCCUCAA | 14597 | TCCAAAATACTTTGTCCTCAA |
| 8933 | UCCAACUCCUGAACUCAAGUG | 14598 | TCCAACTCCTGAACTCAAGTG |
| 8934 | UCCAAGAUGGUCUUCCGGUUG | 14599 | TCCAAGATGGTCTTCCGGTTG |
| 8935 | UCCAAGCAGUCCCGGUCUGAG | 14600 | TCCAAGCAGTCCCGGTCTGAG |
| 8936 | UCCAAGCAUUCGUUGGUCCCG | 14601 | TCCAAGCATTCGTTGGTCCCG |
| 8937 | UCCACACCAUUCAGGCCCCCU | 14602 | TCCACACCATTCAGGCCCCCT |
| 8938 | UCCACAGCCAGCCCGUCGGGG | 14603 | TCCACAGCCAGCCCGTCGGGG |
| 8939 | UCCACCACGAUGGCCCUUGGC | 14604 | TCCACCACGATGGCCCTTGGC |
| 8940 | UCCACCCGCCUCGGCCUCCCA | 14605 | TCCACCCGCCTCGGCCTCCCA |
| 8941 | UCCACCGUGGUGAGCCCAGGG | 14606 | TCCACCGTGGTGAGCCCAGGG |
| 8942 | UCCACCUCCUGGGCUCAGGCG | 14607 | TCCACCTCCTGGGCTCAGGCG |
| 8943 | UCCACUUGGCCAUCGCACCUC | 14608 | TCCACTTGGCCATCGCACCTC |
| 8944 | UCCAGAACUGAGGAAUGCAGC | 14609 | TCCAGAACTGAGGAATGCAGC |
| 8945 | UCCAGACUGACCAUCGAGGGG | 14610 | TCCAGACTGACCATCGAGGGG |
| 8946 | UCCAGACUGACCAUCUGUCUC | 14611 | TCCAGACTGACCATCTGTCTC |
| 8947 | UCCAGACUGGAGGAUUUAUUU | 14612 | TCCAGACTGGAGGATTTATTT |
| 8948 | UCCAGAGCGACCACGUUCCUC | 14613 | TCCAGAGCGACCACGTTCCTC |
| 8949 | UCCAGCCCCAGGGCCCCAUGC | 14614 | TCCAGCCCCAGGGCCCCATGC |
| 8950 | UCCAGCGCAAUUUCCAGCCCC | 14615 | TCCAGCGCAATTTCCAGCCCC |
| 8951 | UCCAGCGUCAUCUUCCUGACC | 14616 | TCCAGCGTCATCTTCCTGACC |
| 8952 | UCCAGCUCAGCCUCCCAAAGU | 14617 | TCCAGCTCAGCCTCCCAAAGT |
| 8953 | UCCAGCUGGAAGCCUUCCUCA | 14618 | TCCAGCTGGAAGCCTTCCTCA |
| 8954 | UCCAGGCAGAUGUUCACGCCA | 14619 | TCCAGGCAGATGTTCACGCCA |
| 8955 | UCCAGGGUGAUGCAUUCGCCG | 14620 | TCCAGGGTGATGCATTCGCCG |
| 8956 | UCCAGGUUCACGCAGAGCUGG | 14621 | TCCAGGTTCACGCAGAGCTGG |
| 8957 | UCCAGUACAUGAAGCCAUGAA | 14622 | TCCAGTACATGAAGCCATGAA |
| 8958 | UCCAGUAGAUGUUGCUGUGGA | 14623 | TCCAGTAGATGTTGCTGTGGA |
| 8959 | UCCAGUCCACAGCCAGCCCGU | 14624 | TCCAGTCCACAGCCAGCCCGT |
| 8960 | UCCAGUUCGUGCAAAUAAUCU | 14625 | TCCAGTTCGTGCAAATAATCT |
| 8961 | UCCAUAGAAGGAAGACCCCCA | 14626 | TCCATAGAAGGAAGACCCCCA |
| 8962 | UCCAUCAGAGCACUGGAAUUC | 14627 | TCCATCAGAGCACTGGAATTC |
| 8963 | UCCAUGUUUGAGGAUUGUGUC | 14628 | TCCATGTTTGAGGATTGTGTC |
| 8964 | UCCCAAAAAGCGUUGCACACA | 14629 | TCCCAAAAAGCGTTGCACACA |
| 8965 | UCCCAAAGUGCUGGGAUUACA | 14630 | TCCCAAAGTGCTGGGATTACA |
| 8966 | UCCCAACACACACGACAGAAA | 14631 | TCCCAACACACACGACAGAAA |
| 8967 | UCCCAACCCAAGCCAUUGCAU | 14632 | TCCCAACCCAAGCCATTGCAT |
| 8968 | UCCCAAGUAGCUGGGAUCACA | 14633 | TCCCAAGTAGCTGGGATCACA |
| 8969 | UCCCACCUCAGCCUCGCCAUG | 14634 | TCCCACCTCAGCCTCGCCATG |
| 8970 | UCCCAGAAGCCACUCAUACAU | 14635 | TCCCAGAAGCCACTCATACAT |
| 8971 | UCCCAGAUGAAUAAAUAUAUA | 14636 | TCCCAGATGAATAAATATATA |
| 8972 | UCCCAUCCCAACACACACGAC | 14637 | TCCCATCCCAACACACACGAC |
| 8973 | UCCCAUCGUGGCAGCGAAACU | 14638 | TCCCATCGTGGCAGCGAAACT |
| 8974 | UCCCCACUAUCUGUACAGGGA | 14639 | TCCCCACTATCTGTACAGGGA |
| 8975 | UCCCCAGUCAGUCCAGUACAU | 14640 | TCCCCAGTCAGTCCAGTACAT |
| 8976 | UCCCCCCGGGCCUGUUUCUCU | 14641 | TCCCCCCGGGCCTGTTTCTCT |
| 8977 | UCCCCGCCGCGGCGAGGAGCA | 14642 | TCCCCGCCGCGGCGAGGAGCA |
| 8978 | UCCCCGGAUUUGCAGGUGACA | 14643 | TCCCCGGATTTGCAGGTGACA |
| 8979 | UCCCCGGCAUUCAUUGACACG | 14644 | TCCCCGGCATTCATTGACACG |
| 8980 | UCCCCUCCCCUUUCAUCUUCC | 14645 | TCCCCTCCCCTTTCATCTTCC |
| 8981 | UCCCCUUGGAACACGUAAAGA | 14646 | TCCCCTTGGAACACGTAAAGA |
| 8982 | UCCCCUUUCAUCUUCCAUGUU | 14647 | TCCCCTTTCATCTTCCATGTT |
| 8983 | UCCCGACCCGGAUCACGACCU | 14648 | TCCCGACCCGGATCACGACCT |
| 8984 | UCCCGAGUAGCUGGGACUACA | 14649 | TCCCGAGTAGCTGGGACTACA |
| 8985 | UCCCGCACUCUUUGAUGGGUU | 14650 | TCCCGCACTCTTTGATGGGTT |
| 8986 | UCCCGGCAGUCUCUAGCCAUG | 14651 | TCCCGGCAGTCTCTAGCCATG |
| 8987 | UCCCGGGUUCACACCAUUCUC | 14652 | TCCCGGGTTCACACCATTCTC |
| 8988 | UCCCGGUCAACCUGCCCCUCU | 14653 | TCCCGGTCAACCTGCCCCTCT |
| 8989 | UCCCGGUCACACUGCCGGCUG | 14654 | TCCCGGTCACACTGCCGGCTG |
| 8990 | UCCCGGUCUGAGUCACAGACG | 14655 | TCCCGGTCTGAGTCACAGACG |
| 8991 | UCCCGUCAAACGAUCCAGACU | 14656 | TCCCGTCAAACGATCCAGACT |
| 8992 | UCCCGUCUUGGCACUGGAACU | 14657 | TCCCGTCTTGGCACTGGAACT |
| 8993 | UCCCGUUGGUGCCAUCUGCUG | 14658 | TCCCGTTGGTGCCATCTGCTG |
| 8994 | UCCCUCGCAGAGUGUCACAUU | 14659 | TCCCTCGCAGAGTGTCACATT |
| 8995 | UCCCUCGCAGAGUGUCAGACA | 14660 | TCCCTCGCAGAGTGTCAGACA |
| 8996 | UCCCUGAAUAACGUUUUCCUC | 14661 | TCCCTGAATAACGTTTTCCTC |
| 8997 | UCCCUGCACUGGGGGGGACAC | 14662 | TCCCTGCACTGGGGGGGACAC |
| 8998 | UCCCUGGCCAGCAGCAUGCCG | 14663 | TCCCTGGCCAGCAGCATGCCG |
| 8999 | UCCCUGGCCUGGGAUCCCAUC | 14664 | TCCCTGGCCTGGGATCCCATC |
| 9000 | UCCCUGGGGACAAUUGCCAAU | 14665 | TCCCTGGGGACAATTGCCAAT |
| 9001 | UCCCUUGUGACAUCUUCACGC | 14666 | TCCCTTGTGACATCTTCACGC |
| 9002 | UCCCUUUCCUGGCUUAAGGAA | 14667 | TCCCTTTCCTGGCTTAAGGAA |
| 9003 | UCCGAAACCAGAAAGGCUUAU | 14668 | TCCGAAACCAGAAAGGCTTAT |
| 9004 | UCCGAAGCCUGUUCUGCCUCC | 14669 | TCCGAAGCCTGTTCTGCCTCC |
| 9005 | UCCGACGCCAUGAAUCCCUUU | 14670 | TCCGACGCCATGAATCCCTTT |
| 9006 | UCCGAGCCAUCUUCGCAGUCG | 14671 | TCCGAGCCATCTTCGCAGTCG |
| 9007 | UCCGCAACAGAGACAGUGCCC | 14672 | TCCGCAACAGAGACAGTGCCC |
| 9008 | UCCGCUUCCCGGGUUCACACC | 14673 | TCCGCTTCCCGGGTTCACACC |
| 9009 | UCCGGACAUCAGUGCAACAGU | 14674 | TCCGGACATCAGTGCAACAGT |
| 9010 | UCCGGGCAGGCGCAGGUAAAC | 14675 | TCCGGGCAGGCGCAGGTAAAC |
| 9011 | UCCGGGGACCCGGGACUUGUC | 14676 | TCCGGGGACCCGGGACTTGTC |
| 9012 | UCCGGUCCAGCGUCAUCUUCC | 14677 | TCCGGTCCAGCGTCATCTTCC |
| 9013 | UCCGGUUGCCCCCGUUGACAU | 14678 | TCCGGTTGCCCCCGTTGACAT |
| 9014 | UCCGUGGCGAUGGGGAGGACA | 14679 | TCCGTGGCGATGGGGAGGACA |
| 9015 | UCCGUGUCCAGAGCGACCACG | 14680 | TCCGTGTCCAGAGCGACCACG |
| 9016 | UCCUAGCUGGAAACCCUGGCU | 14681 | TCCTAGCTGGAAACCCTGGCT |
| 9017 | UCCUCAAAGACGGCCAAGGAG | 14682 | TCCTCAAAGACGGCCAAGGAG |
| 9018 | UCCUCACACUGGCACUUGUAG | 14683 | TCCTCACACTGGCACTTGTAG |
| 9019 | UCCUCACGGCCAAGGUAACCG | 14684 | TCCTCACGGCCAAGGTAACCG |
| 9020 | UCCUCAGGUUGGGGAUGAGGC | 14685 | TCCTCAGGTTGGGGATGAGGC |
| 9021 | UCCUCAUGUCCCUGGCCAGCA | 14686 | TCCTCATGTCCCTGGCCAGCA |
| 9022 | UCCUCCAAGAUGGUCUUCCGG | 14687 | TCCTCCAAGATGGTCTTCCGG |
| 9023 | UCCUCCAGACUGACCAUCGAG | 14688 | TCCTCCAGACTGACCATCGAG |
| 9024 | UCCUCCAGACUGACCAUCUGU | 14689 | TCCTCCAGACTGACCATCTGT |
| 9025 | UCCUCCCACCUCAGCCUCGCC | 14690 | TCCTCCCACCTCAGCCTCGCC |
| 9026 | UCCUCCUGCCUCAGCCUCCCA | 14691 | TCCTCCTGCCTCAGCCTCCCA |
| 9027 | UCCUCGUCAGAUUUGUCCUUG | 14692 | TCCTCGTCAGATTTGTCCTTG |
| 9028 | UCCUCUCACACCAGUUCACUC | 14693 | TCCTCTCACACCAGTTCACTC |
| 9029 | UCCUCUGCCAGCAACGUCGCC | 14694 | TCCTCTGCCAGCAACGTCGCC |
| 9030 | UCCUCUGGGGACAGUAGGUUU | 14695 | TCCTCTGGGGACAGTAGGTTT |
| 9031 | UCCUCUGUGGUCUUCUGAUAG | 14696 | TCCTCTGTGGTCTTCTGATAG |
| 9032 | UCCUCUUCACGCCCUUGGUAU | 14697 | TCCTCTTCACGCCCTTGGTAT |
| 9033 | UCCUCUUGGCUGGGUGAGGUU | 14698 | TCCTCTTGGCTGGGTGAGGTT |
| 9034 | UCCUGAACUCAAGUGAUCCAG | 14699 | TCCTGAACTCAAGTGATCCAG |
| 9035 | UCCUGACACUCAUCGAUAUCG | 14700 | TCCTGACACTCATCGATATCG |
| 9036 | UCCUGACACUCAUCGAUAUCU | 14701 | TCCTGACACTCATCGATATCT |
| 9037 | UCCUGACAGCCACACCUGGGU | 14702 | TCCTGACAGCCACACCTGGGT |
| 9038 | UCCUGACCUCAUGAUCCACCC | 14703 | TCCTGACCTCATGATCCACCC |
| 9039 | UCCUGACCUCGUGCCGGUUGG | 14704 | TCCTGACCTCGTGCCGGTTGG |
| 9040 | UCCUGAGUUUAGGGGUUUGGC | 14705 | TCCTGAGTTTAGGGGTTTGGC |
| 9041 | UCCUGCCUCAGCCUCCCAAGU | 14706 | TCCTGCCTCAGCCTCCCAAGT |
| 9042 | UCCUGCCUCAGCCUCCCGAGU | 14707 | TCCTGCCTCAGCCTCCCGAGT |
| 9043 | UCCUGGAGAGAAAUGGAGGUG | 14708 | TCCTGGAGAGAAATGGAGGTG |
| 9044 | UCCUGGCACUCAGCGCUGCCA | 14709 | TCCTGGCACTCAGCGCTGCCA |
| 9045 | UCCUGGCUUAAGGAAGGUACA | 14710 | TCCTGGCTTAAGGAAGGTACA |
| 9046 | UCCUGGGACUCAUCAGAGCCA | 14711 | TCCTGGGACTCATCAGAGCCA |
| 9047 | UCCUGGGAGCACGUCUUGGGG | 14712 | TCCTGGGAGCACGTCTTGGGG |
| 9048 | UCCUGGGCUCAGGCGAUCCUC | 14713 | TCCTGGGCTCAGGCGATCCTC |
| 9049 | UCCUGGGUGGCCACUGCAGCC | 14714 | TCCTGGGTGGCCACTGCAGCC |
| 9050 | UCCUGGUCCCCCCGGGCCUGU | 14715 | TCCTGGTCCCCCCGGGCCTGT |
| 9051 | UCCUGGUUGUGGCAAAUGUGG | 14716 | TCCTGGTTGTGGCAAATGTGG |
| 9052 | UCCUUACGGCUGUGGAGCUGA | 14717 | TCCTTACGGCTGTGGAGCTGA |
| 9053 | UCCUUCACGAGGAAAGGAAGA | 14718 | TCCTTCACGAGGAAAGGAAGA |
| 9054 | UCCUUGCAGUCAUAUUCCCGG | 14719 | TCCTTGCAGTCATATTCCCGG |
| 9055 | UCCUUGCAGUCGGGGCCACCA | 14720 | TCCTTGCAGTCGGGGCCACCA |
| 9056 | UCGAAAGACACUGGGUGGGUC | 14721 | TCGAAAGACACTGGGTGGGTC |
| 9057 | UCGAAGGCCGAGCAGGGGCUA | 14722 | TCGAAGGCCGAGCAGGGGCTA |
| 9058 | UCGAGGGGUAGCUGUAGCCGU | 14723 | TCGAGGGGTAGCTGTAGCCGT |
| 9059 | UCGAUAUCGCACUCUUUGAUG | 14724 | TCGATATCGCACTCTTTGATG |
| 9060 | UCGAUAUCUUCGCAUCUUCGC | 14725 | TCGATATCTTCGCATCTTCGC |
| 9061 | UCGAUAUUCACGAUCACGGCU | 14726 | TCGATATTCACGATCACGGCT |
| 9062 | UCGAUCAUGGCUCACUGCAAC | 14727 | TCGATCATGGCTCACTGCAAC |
| 9063 | UCGAUCUCCUGACCUCAUGAU | 14728 | TCGATCTCCTGACCTCATGAT |
| 9064 | UCGAUGCUUGAGAUGGAGUGA | 14729 | TCGATGCTTGAGATGGAGTGA |
| 9065 | UCGCACCUCCAGAACUGAGGA | 14730 | TCGCACCTCCAGAACTGAGGA |
| 9066 | UCGCACUCUUUGAUGGGUUCA | 14731 | TCGCACTCTTTGATGGGTTCA |
| 9067 | UCGCAGACCCACUUGUAGGAG | 14732 | TCGCAGACCCACTTGTAGGAG |
| 9068 | UCGCAGAGUGUCACAUUAACG | 14733 | TCGCAGAGTGTCACATTAACG |
| 9069 | UCGCAGAGUGUCAGACAGCCU | 14734 | TCGCAGAGTGTCAGACAGCCT |
| 9070 | UCGCAGCCUCUGCCAGGCAGU | 14735 | TCGCAGCCTCTGCCAGGCAGT |
| 9071 | UCGCAGGCCCACAGCUGGGGG | 14736 | TCGCAGGCCCACAGCTGGGGG |
| 9072 | UCGCAGUCCACUUGGCCAUCG | 14737 | TCGCAGTCCACTTGGCCATCG |
| 9073 | UCGCAGUCGGGGUCGUUGUCG | 14738 | TCGCAGTCGGGGTCGTTGTCG |
| 9074 | UCGCAUCUGUCGCCCACGUGG | 14739 | TCGCATCTGTCGCCCACGTGG |
| 9075 | UCGCAUCUGUCGCCCACUGCA | 14740 | TCGCATCTGTCGCCCACTGCA |
| 9076 | UCGCAUCUUCGCUGGGCCACC | 14741 | TCGCATCTTCGCTGGGCCACC |
| 9077 | UCGCCACAGAGCACAGCGGAA | 14742 | TCGCCACAGAGCACAGCGGAA |
| 9078 | UCGCCACUUAGGCAGUGGAAC | 14743 | TCGCCACTTAGGCAGTGGAAC |
| 9079 | UCGCCAGGAGGGCCCAGGAUA | 14744 | TCGCCAGGAGGGCCCAGGATA |
| 9080 | UCGCCAUGCCUGGCCAGAGGC | 14745 | TCGCCATGCCTGGCCAGAGGC |
| 9081 | UCGCCCACGUGGCUGAAGAUC | 14746 | TCGCCCACGTGGCTGAAGATC |
| 9082 | UCGCCCACUGCAGUCCCCGCC | 14747 | TCGCCCACTGCAGTCCCCGCC |
| 9083 | UCGCCCAGAGCUUGGUGAGAC | 14748 | TCGCCCAGAGCTTGGTGAGAC |
| 9084 | UCGCCCAGGCCAGACUGCGGA | 14749 | TCGCCCAGGCCAGACTGCGGA |
| 9085 | UCGCCGCUGUGACACUUGAAC | 14750 | TCGCCGCTGTGACACTTGAAC |
| 9086 | UCGCCUUUGUGACAGGAACCG | 14751 | TCGCCTTTGTGACAGGAACCG |
| 9087 | UCGCGGAGGCGGCUCCGGGGA | 14752 | TCGCGGAGGCGGCTCCGGGGA |
| 9088 | UCGCUCAUGUCCUUGCAGUCA | 14753 | TCGCTCATGTCCTTGCAGTCA |
| 9089 | UCGCUCCGGUCCAGCGUCAUC | 14754 | TCGCTCCGGTCCAGCGTCATC |
| 9090 | UCGCUCUGUCGCCCAGGCCAG | 14755 | TCGCTCTGTCGCCCAGGCCAG |
| 9091 | UCGCUGGGCCACCAGCUGGAA | 14756 | TCGCTGGGCCACCAGCTGGAA |
| 9092 | UCGCUUUGUUGCUCAGGCCUG | 14757 | TCGCTTTGTTGCTCAGGCCTG |
| 9093 | UCGGAACCUGUGAGGCGGUUG | 14758 | TCGGAACCTGTGAGGCGGTTG |
| 9094 | UCGGCCUCCCAAAGUGCUGGG | 14759 | TCGGCCTCCCAAAGTGCTGGG |
| 9095 | UCGGCUCACUGCAAGCUCCGC | 14760 | TCGGCTCACTGCAAGCTCCGC |
| 9096 | UCGGCUGCAGACCAAGGAGGG | 14761 | TCGGCTGCAGACCAAGGAGGG |
| 9097 | UCGGGAACAGGUCGGGUGGUU | 14762 | TCGGGAACAGGTCGGGTGGTT |
| 9098 | UCGGGAGGAUGGGCACCAGGC | 14763 | TCGGGAGGATGGGCACCAGGC |
| 9099 | UCGGGAUCCUGACACUCAUCG | 14764 | TCGGGATCCTGACACTCATCG |
| 9100 | UCGGGGCACAGGCACUCGUAG | 14765 | TCGGGGCACAGGCACTCGTAG |
| 9101 | UCGGGGCCACCAUCACAGCGC | 14766 | TCGGGGCCACCATCACAGCGC |
| 9102 | UCGGGGCUGGUGGGGCGAGGG | 14767 | TCGGGGCTGGTGGGGCGAGGG |
| 9103 | UCGGGGGCCUGGAUGUCUCUG | 14768 | TCGGGGGCCTGGATGTCTCTG |
| 9104 | UCGGGGGUGACAGGGCAAAGG | 14769 | TCGGGGGTGACAGGGCAAAGG |
| 9105 | UCGGGGUCGUUGUCGCAGGCC | 14770 | TCGGGGTCGTTGTCGCAGGCC |
| 9106 | UCGGGUGGUUGUGUGCUGUGU | 14771 | TCGGGTGGTTGTGTGCTGTGT |
| 9107 | UCGGUCCAGUAGAUGUUGCUG | 14772 | TCGGTCCAGTAGATGTTGCTG |
| 9108 | UCGGUGCCACCACGGAUUCAG | 14773 | TCGGTGCCACCACGGATTCAG |
| 9109 | UCGUAAGGAACCGAGGCCUUG | 14774 | TCGTAAGGAACCGAGGCCTTG |
| 9110 | UCGUAGCCGAUCUUAAGGUCA | 14775 | TCGTAGCCGATCTTAAGGTCA |
| 9111 | UCGUCAGAUUUGUCCUUGCAG | 14776 | TCGTCAGATTTGTCCTTGCAG |
| 9112 | UCGUCAGGGCGACAGGUGGCC | 14777 | TCGTCAGGGCGACAGGTGGCC |
| 9113 | UCGUCCUGGGAGCACGUCUUG | 14778 | TCGTCCTGGGAGCACGTCTTG |
| 9114 | UCGUCGCCUUUGUGACAGGAA | 14779 | TCGTCGCCTTTGTGACAGGAA |
| 9115 | UCGUCUGAGCCGUCCAAGCAG | 14780 | TCGTCTGAGCCGTCCAAGCAG |
| 9116 | UCGUCUGAGCCGUUGUCGCAG | 14781 | TCGTCTGAGCCGTTGTCGCAG |
| 9117 | UCGUGCAAAUAAUCUUUGUAC | 14782 | TCGTGCAAATAATCTTTGTAC |
| 9118 | UCGUGCCGGUUGGUGAAGAAG | 14783 | TCGTGCCGGTTGGTGAAGAAG |
| 9119 | UCGUGGCAGCGAAACUCGUCC | 14784 | TCGTGGCAGCGAAACTCGTCC |
| 9120 | UCGUGUAUGUCGCGGAGGCGG | 14785 | TCGTGTATGTCGCGGAGGCGG |
| 9121 | UCGUGUGGGGGUCCAGCUGGA | 14786 | TCGTGTGGGGGTCCAGCTGGA |
| 9122 | UCGUUGAUGAUAUCUGUCCAA | 14787 | TCGTTGATGATATCTGTCCAA |
| 9123 | UCGUUGGUCCCGCACUCUUUG | 14788 | TCGTTGGTCCCGCACTCTTTG |
| 9124 | UCGUUGUCGCAGGCCCACAGC | 14789 | TCGTTGTCGCAGGCCCACAGC |
| 9125 | UCGUUUCUUUCGCAUCUGUCG | 14790 | TCGTTTCTTTCGCATCTGTCG |
| 9126 | UCUAGCAAGGCUUGCAUAGAA | 14791 | TCTAGCAAGGCTTGCATAGAA |
| 9127 | UCUAGCCAUGUUGCAGACUUU | 14792 | TCTAGCCATGTTGCAGACTTT |
| 9128 | UCUAGGGUGAUGCCAUUGGGC | 14793 | TCTAGGGTGATGCCATTGGGC |
| 9129 | UCUAUACAGUGGUUUAAAAAG | 14794 | TCTATACAGTGGTTTAAAAAG |
| 9130 | UCUAUUGCUGGCCACCUCCGU | 14795 | TCTATTGCTGGCCACCTCCGT |
| 9131 | UCUCACACCAGUUCACUCCUC | 14796 | TCTCACACCAGTTCACTCCTC |
| 9132 | UCUCAGGAAGGGUUCUGGGCA | 14797 | TCTCAGGAAGGGTTCTGGGCA |
| 9133 | UCUCAGGCACUUAAUAAAUAU | 14798 | TCTCAGGCACTTAATAAATAT |
| 9134 | UCUCAUUUCCUCUGCCAGCAA | 14799 | TCTCATTTCCTCTGCCAGCAA |
| 9135 | UCUCCAACUCCUGAACUCAAG | 14800 | TCTCCAACTCCTGAACTCAAG |
| 9136 | UCUCCACCGUGGUGAGCCCAG | 14801 | TCTCCACCGTGGTGAGCCCAG |
| 9137 | UCUCCCAAAAAGCGUUGCACA | 14802 | TCTCCCAAAAAGCGTTGCACA |
| 9138 | UCUCCCAGAAGCCACUCAUAC | 14803 | TCTCCCAGAAGCCACTCATAC |
| 9139 | UCUCCCUGAAUAACGUUUUCC | 14804 | TCTCCCTGAATAACGTTTTCC |
| 9140 | UCUCCGGACAUCAGUGCAACA | 14805 | TCTCCGGACATCAGTGCAACA |
| 9141 | UCUCCUGACCUCAUGAUCCAC | 14806 | TCTCCTGACCTCATGATCCAC |
| 9142 | UCUCCUGCCUCAGCCUCCCGA | 14807 | TCTCCTGCCTCAGCCTCCCGA |
| 9143 | UCUCCUGGGACUCAUCAGAGC | 14808 | TCTCCTGGGACTCATCAGAGC |
| 9144 | UCUCCUGGGUGGCCACUGCAG | 14809 | TCTCCTGGGTGGCCACTGCAG |
| 9145 | UCUCGAGGGGUAGCUGUAGCC | 14810 | TCTCGAGGGGTAGCTGTAGCC |
| 9146 | UCUCGAUAUUCACGAUCACGG | 14811 | TCTCGATATTCACGATCACGG |
| 9147 | UCUCGAUCUCCUGACCUCAUG | 14812 | TCTCGATCTCCTGACCTCATG |
| 9148 | UCUCGCUCUGUCGCCCAGGCC | 14813 | TCTCGCTCTGTCGCCCAGGCC |
| 9149 | UCUCGCUUUGUUGCUCAGGCC | 14814 | TCTCGCTTTGTTGCTCAGGCC |
| 9150 | UCUCGGCUCACUGCAAGCUCC | 14815 | TCTCGGCTCACTGCAAGCTCC |
| 9151 | UCUCGGCUGCAGACCAAGGAG | 14816 | TCTCGGCTGCAGACCAAGGAG |
| 9152 | UCUCGGUGCCACCACGGAUUC | 14817 | TCTCGGTGCCACCACGGATTC |
| 9153 | UCUCUAGCCAUGUUGCAGACU | 14818 | TCTCTAGCCATGTTGCAGACT |
| 9154 | UCUCUCCGGACAUCAGUGCAA | 14819 | TCTCTCCGGACATCAGTGCAA |
| 9155 | UCUCUGCUGAUGACGGUGUCA | 14820 | TCTCTGCTGATGACGGTGTCA |
| 9156 | UCUCUGGGACAGGUCAGACCA | 14821 | TCTCTGGGACAGGTCAGACCA |
| 9157 | UCUCUGGGCCUGACAUCCCCU | 14822 | TCTCTGGGCCTGACATCCCCT |
| 9158 | UCUCUGUCCCCGGCAUUCAUU | 14823 | TCTCTGTCCCCGGCATTCATT |
| 9159 | UCUCUGUCUUUGAAUAAAACA | 14824 | TCTCTGTCTTTGAATAAAACA |
| 9160 | UCUCUUAUCCUUCACGAGGAA | 14825 | TCTCTTATCCTTCACGAGGAA |
| 9161 | UCUCUUGGCUGGGUGAGGUUG | 14826 | TCTCTTGGCTGGGTGAGGTTG |
| 9162 | UCUGAAAUAGGGUCUUCAUCU | 14827 | TCTGAAATAGGGTCTTCATCT |
| 9163 | UCUGAACUGAGAAAGUGCAAG | 14828 | TCTGAACTGAGAAAGTGCAAG |
| 9164 | UCUGACCAGUCCCGGCAGUCU | 14829 | TCTGACCAGTCCCGGCAGTCT |
| 9165 | UCUGACGGAGGCUGUCACUGU | 14830 | TCTGACGGAGGCTGTCACTGT |
| 9166 | UCUGAGCCGUCCAAGCAGUCC | 14831 | TCTGAGCCGTCCAAGCAGTCC |
| 9167 | UCUGAGCCGUUGUCGCAGUCC | 14832 | TCTGAGCCGTTGTCGCAGTCC |
| 9168 | UCUGAGGCAACGUUUCAACCU | 14833 | TCTGAGGCAACGTTTCAACCT |
| 9169 | UCUGAGUCACAGACGAACUGC | 14834 | TCTGAGTCACAGACGAACTGC |
| 9170 | UCUGAUAGACGGGGUUGUCAA | 14835 | TCTGATAGACGGGGTTGTCAA |
| 9171 | UCUGCCAGCAACGUCGCCCAG | 14836 | TCTGCCAGCAACGTCGCCCAG |
| 9172 | UCUGCCAGGCAGUGUCCCGAC | 14837 | TCTGCCAGGCAGTGTCCCGAC |
| 9173 | UCUGCCUCCCAGAUGAAUAAA | 14838 | TCTGCCTCCCAGATGAATAAA |
| 9174 | UCUGCCUCUGGGAAGUGGCAU | 14839 | TCTGCCTCTGGGAAGTGGCAT |
| 9175 | UCUGCGGGGCAGGGAGGCACA | 14840 | TCTGCGGGGCAGGGAGGCACA |
| 9176 | UCUGCUGAUGACGGUGUCAUA | 14841 | TCTGCTGATGACGGTGTCATA |
| 9177 | UCUGCUGUUGUGUGUUGAAAG | 14842 | TCTGCTGTTGTGTGTTGAAAG |
| 9178 | UCUGGCAGGCAAUGCUUUGGU | 14843 | TCTGGCAGGCAATGCTTTGGT |
| 9179 | UCUGGCCAAAUUCAUUUUUUA | 14844 | TCTGGCCAAATTCATTTTTTA |
| 9180 | UCUGGGAAGUGGCAUCAUUUG | 14845 | TCTGGGAAGTGGCATCATTTG |
| 9181 | UCUGGGACAGGUCAGACCAGU | 14846 | TCTGGGACAGGTCAGACCAGT |
| 9182 | UCUGGGCAGGGACGGGACUCC | 14847 | TCTGGGCAGGGACGGGACTCC |
| 9183 | UCUGGGCCUGACAUCCCCUCC | 14848 | TCTGGGCCTGACATCCCCTCC |
| 9184 | UCUGGGGACAGUAGGUUUUCA | 14849 | TCTGGGGACAGTAGGTTTTCA |
| 9185 | UCUGUACAGGGACGCAUUUAC | 14850 | TCTGTACAGGGACGCATTTAC |
| 9186 | UCUGUCAAGCUGGGUGCUGCA | 14851 | TCTGTCAAGCTGGGTGCTGCA |
| 9187 | UCUGUCACACAGCUGCCCCCG | 14852 | TCTGTCACACAGCTGCCCCCG |
| 9188 | UCUGUCCAAAAUACUUUGUCC | 14853 | TCTGTCCAAAATACTTTGTCC |
| 9189 | UCUGUCCCCGGCAUUCAUUGA | 14854 | TCTGTCCCCGGCATTCATTGA |
| 9190 | UCUGUCGCCCACGUGGCUGAA | 14855 | TCTGTCGCCCACGTGGCTGAA |
| 9191 | UCUGUCGCCCACUGCAGUCCC | 14856 | TCTGTCGCCCACTGCAGTCCC |
| 9192 | UCUGUCGCCCAGGCCAGACUG | 14857 | TCTGTCGCCCAGGCCAGACTG |
| 9193 | UCUGUCUCGAGGGGUAGCUGU | 14858 | TCTGTCTCGAGGGGTAGCTGT |
| 9194 | UCUGUCUUUGAAUAAAACAAG | 14859 | TCTGTCTTTGAATAAAACAAG |
| 9195 | UCUGUGAACUUGAUCGGGGCU | 14860 | TCTGTGAACTTGATCGGGGCT |
| 9196 | UCUGUGAGGCAGCUCCUCAUG | 14861 | TCTGTGAGGCAGCTCCTCATG |
| 9197 | UCUGUGGUCUUCUGAUAGACG | 14862 | TCTGTGGTCTTCTGATAGACG |
| 9198 | UCUGUUGCCCAGGCUGGAGUG | 14863 | TCTGTTGCCCAGGCTGGAGTG |
| 9199 | UCUUAAGCCGCCAGUUCUUCC | 14864 | TCTTAAGCCGCCAGTTCTTCC |
| 9200 | UCUUAAGGUCAUUGCAGACGU | 14865 | TCTTAAGGTCATTGCAGACGT |
| 9201 | UCUUACUAUGUUGCCCAGGCU | 14866 | TCTTACTATGTTGCCCAGGCT |
| 9202 | UCUUAUCCUUCACGAGGAAAG | 14867 | TCTTATCCTTCACGAGGAAAG |
| 9203 | UCUUCACGCCCUUGGUAUCCG | 14868 | TCTTCACGCCCTTGGTATCCG |
| 9204 | UCUUCACGCGGGAGUCUGACG | 14869 | TCTTCACGCGGGAGTCTGACG |
| 9205 | UCUUCAUCUGUUGCCCAGGCU | 14870 | TCTTCATCTGTTGCCCAGGCT |
| 9206 | UCUUCCAUAGAAGGAAGACCC | 14871 | TCTTCCATAGAAGGAAGACCC |
| 9207 | UCUUCCAUGUUUGAGGAUUGU | 14872 | TCTTCCATGTTTGAGGATTGT |
| 9208 | UCUUCCGGUUGCCCCCGUUGA | 14873 | TCTTCCGGTTGCCCCCGTTGA |
| 9209 | UCUUCCUGACCUCGUGCCGGU | 14874 | TCTTCCTGACCTCGTGCCGGT |
| 9210 | UCUUCGCAGUCGGGGUCGUUG | 14875 | TCTTCGCAGTCGGGGTCGTTG |
| 9211 | UCUUCGCAUCUUCGCUGGGCC | 14876 | TCTTCGCATCTTCGCTGGGCC |
| 9212 | UCUUCGCUGGGCCACCAGCUG | 14877 | TCTTCGCTGGGCCACCAGCTG |
| 9213 | UCUUCUCAUUUCCUCUGCCAG | 14878 | TCTTCTCATTTCCTCTGCCAG |
| 9214 | UCUUCUCUGUCUUUGAAUAAA | 14879 | TCTTCTCTGTCTTTGAATAAA |
| 9215 | UCUUCUGAUAGACGGGGUUGU | 14880 | TCTTCTGATAGACGGGGTTGT |
| 9216 | UCUUGAUCUUGGCGGGAGUUC | 14881 | TCTTGATCTTGGCGGGAGTTC |
| 9217 | UCUUGGCACUGGAACUCGUUU | 14882 | TCTTGGCACTGGAACTCGTTT |
| 9218 | UCUUGGCGGGAGUUCCCCAGU | 14883 | TCTTGGCGGGAGTTCCCCAGT |
| 9219 | UCUUGGCUGGGUGAGGUUGUG | 14884 | TCTTGGCTGGGTGAGGTTGTG |
| 9220 | UCUUGGGGGAGCACGUCUCCU | 14885 | TCTTGGGGGAGCACGTCTCCT |
| 9221 | UCUUGGGGGGACAGCCUUGCU | 14886 | TCTTGGGGGGACAGCCTTGCT |
| 9222 | UCUUUCGCAUCUGUCGCCCAC | 14887 | TCTTTCGCATCTGTCGCCCAC |
| 9223 | UCUUUGAAUAAAACAAGGCCG | 14888 | TCTTTGAATAAAACAAGGCCG |
| 9224 | UCUUUGAUGGGUUCAUCUGAC | 14889 | TCTTTGATGGGTTCATCTGAC |
| 9225 | UCUUUGUACAUAAAGAAGAGG | 14890 | TCTTTGTACATAAAGAAGAGG |
| 9226 | UCUUUUCAUCCUCCAAGAUGG | 14891 | TCTTTTCATCCTCCAAGATGG |
| 9227 | UGAAAAUGGCUUCGUUGAUGA | 14892 | TGAAAATGGCTTCGTTGATGA |
| 9228 | UGAAAGCCACUUGCUUCUCUG | 14893 | TGAAAGCCACTTGCTTCTCTG |
| 9229 | UGAAAUAGGGUCUUCAUCUGU | 14894 | TGAAATAGGGTCTTCATCTGT |
| 9230 | UGAACAAAUACAGCAACCAGG | 14895 | TGAACAAATACAGCAACCAGG |
| 9231 | UGAACAGGAUCCACCACGAUG | 14896 | TGAACAGGATCCACCACGATG |
| 9232 | UGAACUCAAGUGAUCCAGCUC | 14897 | TGAACTCAAGTGATCCAGCTC |
| 9233 | UGAACUGAGAAAGUGCAAGGA | 14898 | TGAACTGAGAAAGTGCAAGGA |
| 9234 | UGAACUUGAUCGGGGCUGGUG | 14899 | TGAACTTGATCGGGGCTGGTG |
| 9235 | UGAACUUGUUGGGUCCCUCGC | 14900 | TGAACTTGTTGGGTCCCTCGC |
| 9236 | UGAAGAAGAGGUAGGCGAUGG | 14901 | TGAAGAAGAGGTAGGCGATGG |
| 9237 | UGAAGAUCUCGGCUGCAGACC | 14902 | TGAAGATCTCGGCTGCAGACC |
| 9238 | UGAAGCCAUGAACAGGAUCCA | 14903 | TGAAGCCATGAACAGGATCCA |
| 9239 | UGAAGUCCCCGGAUUUGCAGG | 14904 | TGAAGTCCCCGGATTTGCAGG |
| 9240 | UGAAGUCCCGGUCAACCUGCC | 14905 | TGAAGTCCCGGTCAACCTGCC |
| 9241 | UGAAGUCCCGUCAAACGAUCC | 14906 | TGAAGTCCCGTCAAACGATCC |
| 9242 | UGAAGUUUGGAGUCAACCCAG | 14907 | TGAAGTTTGGAGTCAACCCAG |
| 9243 | UGAAUAAAACAAGGCCGGCGA | 14908 | TGAATAAAACAAGGCCGGCGA |
| 9244 | UGAAUAAAUAUAUAAAACAAA | 14909 | TGAATAAATATATAAAACAAA |
| 9245 | UGAAUAACGUUUUCCUCUUCA | 14910 | TGAATAACGTTTTCCTCTTCA |
| 9246 | UGAAUCCCUUUCCUGGCUUAA | 14911 | TGAATCCCTTTCCTGGCTTAA |
| 9247 | UGAAUGAGUUUGGUCUCGGUG | 14912 | TGAATGAGTTTGGTCTCGGTG |
| 9248 | UGAAUGGCUUGGAGGCAUUUG | 14913 | TGAATGGCTTGGAGGCATTTG |
| 9249 | UGAAUGUUUUCAGUCACCAGC | 14914 | TGAATGTTTTCAGTCACCAGC |
| 9250 | UGACACCCAUCUCCCAGAAGC | 14915 | TGACACCCATCTCCCAGAAGC |
| 9251 | UGACACGGCCCCCACAGCUGA | 14916 | TGACACGGCCCCCACAGCTGA |
| 9252 | UGACACGGGCUUUCCCUGGCC | 14917 | TGACACGGGCTTTCCCTGGCC |
| 9253 | UGACACUCAUCGAUAUCGCAC | 14918 | TGACACTCATCGATATCGCAC |
| 9254 | UGACACUCAUCGAUAUCUUCG | 14919 | TGACACTCATCGATATCTTCG |
| 9255 | UGACACUUGAACUUGUUGGGU | 14920 | TGACACTTGAACTTGTTGGGT |
| 9256 | UGACAGACAAGCACGUCUCCU | 14921 | TGACAGACAAGCACGTCTCCT |
| 9257 | UGACAGCCACACCUGGGUGCA | 14922 | TGACAGCCACACCTGGGTGCA |
| 9258 | UGACAGGAACCGCGGAGGAAC | 14923 | TGACAGGAACCGCGGAGGAAC |
| 9259 | UGACAGGGCAAAGGCUAACCU | 14924 | TGACAGGGCAAAGGCTAACCT |
| 9260 | UGACAUCCCCUCCCCUUUCAU | 14925 | TGACATCCCCTCCCCTTTCAT |
| 9261 | UGACAUCGAUGCUUGAGAUGG | 14926 | TGACATCGATGCTTGAGATGG |
| 9262 | UGACAUCGGAACCUGUGAGGC | 14927 | TGACATCGGAACCTGTGAGGC |
| 9263 | UGACAUCUUCACGCGGGAGUC | 14928 | TGACATCTTCACGCGGGAGTC |
| 9264 | UGACCAGUCCCGGCAGUCUCU | 14929 | TGACCAGTCCCGGCAGTCTCT |
| 9265 | UGACCAGUGACUCAGGCUCUG | 14930 | TGACCAGTGACTCAGGCTCTG |
| 9266 | UGACCAUCGAGGGGUAGCUGU | 14931 | TGACCATCGAGGGGTAGCTGT |
| 9267 | UGACCAUCUGUCUCGAGGGGU | 14932 | TGACCATCTGTCTCGAGGGGT |
| 9268 | UGACCUCAUGAUCCACCCGCC | 14933 | TGACCTCATGATCCACCCGCC |
| 9269 | UGACCUCGUGCCGGUUGGUGA | 14934 | TGACCTCGTGCCGGTTGGTGA |
| 9270 | UGACCUUUAGCCUGACGGUGG | 14935 | TGACCTTTAGCCTGACGGTGG |
| 9271 | UGACGGAGGCUGUCACUGUCU | 14936 | TGACGGAGGCTGTCACTGTCT |
| 9272 | UGACGGUGGAUGUCUCCUGGG | 14937 | TGACGGTGGATGTCTCCTGGG |
| 9273 | UGACGGUGUCAUAGGAAGAGA | 14938 | TGACGGTGTCATAGGAAGAGA |
| 9274 | UGACUCAGGCUCUGCCUCUGG | 14939 | TGACTCAGGCTCTGCCTCTGG |
| 9275 | UGACUCCUGAGUUUAGGGGUU | 14940 | TGACTCCTGAGTTTAGGGGTT |
| 9276 | UGAGAAAGUGCAAGGAGACCA | 14941 | TGAGAAAGTGCAAGGAGACCA |
| 9277 | UGAGACAUUGUCACUAUCUCC | 14942 | TGAGACATTGTCACTATCTCC |
| 9278 | UGAGACGGAGUCUCGCUCUGU | 14943 | TGAGACGGAGTCTCGCTCTGT |
| 9279 | UGAGAUGGAGUGAAGUUUGGA | 14944 | TGAGATGGAGTGAAGTTTGGA |
| 9280 | UGAGCACCGGGCAGGAGGCCU | 14945 | TGAGCACCGGGCAGGAGGCCT |
| 9281 | UGAGCCACUGCGCCCGGCCUC | 14946 | TGAGCCACTGCGCCCGGCCTC |
| 9282 | UGAGCCCAGGGGUGGCCCCAG | 14947 | TGAGCCCAGGGGTGGCCCCAG |
| 9283 | UGAGCCGUCCAAGCAGUCCCG | 14948 | TGAGCCGTCCAAGCAGTCCCG |
| 9284 | UGAGCCGUUGUCGCAGUCCAC | 14949 | TGAGCCGTTGTCGCAGTCCAC |
| 9285 | UGAGCUCAAACCAUCCUCCCA | 14950 | TGAGCTCAAACCATCCTCCCA |
| 9286 | UGAGGAAUGCAGCGGUUGACA | 14951 | TGAGGAATGCAGCGGTTGACA |
| 9287 | UGAGGAGAUCUAGGGUGAUGC | 14952 | TGAGGAGATCTAGGGTGATGC |
| 9288 | UGAGGAUUGUGUCACGGUGGA | 14953 | TGAGGATTGTGTCACGGTGGA |
| 9289 | UGAGGCAACGUUUCAACCUCG | 14954 | TGAGGCAACGTTTCAACCTCG |
| 9290 | UGAGGCACCUGUGUCUGGCCA | 14955 | TGAGGCACCTGTGTCTGGCCA |
| 9291 | UGAGGCAGCUCCUCAUGUCCC | 14956 | TGAGGCAGCTCCTCATGTCCC |
| 9292 | UGAGGCAGGGCCAGGGGGUCC | 14957 | TGAGGCAGGGCCAGGGGGTCC |
| 9293 | UGAGGCGGUUGGCACUGAAAA | 14958 | TGAGGCGGTTGGCACTGAAAA |
| 9294 | UGAGGCUGGUGUACUCGCUCC | 14959 | TGAGGCTGGTGTACTCGCTCC |
| 9295 | UGAGGGUGGCCGGGGGCCCCU | 14960 | TGAGGGTGGCCGGGGGCCCCT |
| 9296 | UGAGGUUGUGGAAGAGAACCA | 14961 | TGAGGTTGTGGAAGAGAACCA |
| 9297 | UGAGUCACAGACGAACUGCCG | 14962 | TGAGTCACAGACGAACTGCCG |
| 9298 | UGAGUUUAGGGGUUUGGCUUA | 14963 | TGAGTTTAGGGGTTTGGCTTA |
| 9299 | UGAGUUUGGUCUCGGUGCCAC | 14964 | TGAGTTTGGTCTCGGTGCCAC |
| 9300 | UGAUAACAGUUUCAACCAAUA | 14965 | TGATAACAGTTTCAACCAATA |
| 9301 | UGAUAGACGGGGUUGUCAAAG | 14966 | TGATAGACGGGGTTGTCAAAG |
| 9302 | UGAUAUCUGUCCAAAAUACUU | 14967 | TGATATCTGTCCAAAATACTT |
| 9303 | UGAUCACAGUAAGACAUUCAG | 14968 | TGATCACAGTAAGACATTCAG |
| 9304 | UGAUCCACCCGCCUCGGCCUC | 14969 | TGATCCACCCGCCTCGGCCTC |
| 9305 | UGAUCCAGCUCAGCCUCCCAA | 14970 | TGATCCAGCTCAGCCTCCCAA |
| 9306 | UGAUCGGGGCUGGUGGGGCGA | 14971 | TGATCGGGGCTGGTGGGGCGA |
| 9307 | UGAUCUGCGGGGCAGGGAGGC | 14972 | TGATCTGCGGGGCAGGGAGGC |
| 9308 | UGAUCUUGGCGGGAGUUCCCC | 14973 | TGATCTTGGCGGGAGTTCCCC |
| 9309 | UGAUGACGGUGUCAUAGGAAG | 14974 | TGATGACGGTGTCATAGGAAG |
| 9310 | UGAUGAUAUCUGUCCAAAAUA | 14975 | TGATGATATCTGTCCAAAATA |
| 9311 | UGAUGCACCAUGCAUGGUUUU | 14976 | TGATGCACCATGCATGGTTTT |
| 9312 | UGAUGCAUUCGCCGCUGUGAC | 14977 | TGATGCATTCGCCGCTGTGAC |
| 9313 | UGAUGCCAUUGGGCCACUGAA | 14978 | TGATGCCATTGGGCCACTGAA |
| 9314 | UGAUGCUGUUGAUGUUCUUAA | 14979 | TGATGCTGTTGATGTTCTTAA |
| 9315 | UGAUGGGUUCAUCUGACCAGU | 14980 | TGATGGGTTCATCTGACCAGT |
| 9316 | UGAUGUUCUUAAGCCGCCAGU | 14981 | TGATGTTCTTAAGCCGCCAGT |
| 9317 | UGAUUCGGGGGUGACAGGGCA | 14982 | TGATTCGGGGGTGACAGGGCA |
| 9318 | UGAUUUUUAACUUUUUGGUUU | 14983 | TGATTTTTAACTTTTTGGTTT |
| 9319 | UGCAAAAAUAAAUAGAUUUUA | 14984 | TGCAAAAATAAATAGATTTTA |
| 9320 | UGCAAAAUAAUAACACAAAUG | 14985 | TGCAAAATAATAACACAAATG |
| 9321 | UGCAAACAUAACAAAAAAUCC | 14986 | TGCAAACATAACAAAAAATCC |
| 9322 | UGCAAAUAAUCUUUGUACAUA | 14987 | TGCAAATAATCTTTGTACATA |
| 9323 | UGCAACAGUAACACGGCGAUU | 14988 | TGCAACAGTAACACGGCGATT |
| 9324 | UGCAACCUCCACCUCCUGGGC | 14989 | TGCAACCTCCACCTCCTGGGC |
| 9325 | UGCAAGCUCCGCUUCCCGGGU | 14990 | TGCAAGCTCCGCTTCCCGGGT |
| 9326 | UGCAAGGAGACCACGGGAAUG | 14991 | TGCAAGGAGACCACGGGAATG |
| 9327 | UGCACACAGUCCAGUUCGUGC | 14992 | TGCACACAGTCCAGTTCGTGC |
| 9328 | UGCACCAUGCAUGGUUUUUUU | 14993 | TGCACCATGCATGGTTTTTTT |
| 9329 | UGCACUCGCCACUUAGGCAGU | 14994 | TGCACTCGCCACTTAGGCAGT |
| 9330 | UGCACUGGAAGCUGGCGGGAC | 14995 | TGCACTGGAAGCTGGCGGGAC |
| 9331 | UGCACUGGGGGGGACACAGUG | 14996 | TGCACTGGGGGGGACACAGTG |
| 9332 | UGCACUUCCCAUCGUGGCAGC | 14997 | TGCACTTCCCATCGTGGCAGC |
| 9333 | UGCAGACCAAGGAGGGGCGGG | 14998 | TGCAGACCAAGGAGGGGCGGG |
| 9334 | UGCAGACGUGGGAACAGCCGC | 14999 | TGCAGACGTGGGAACAGCCGC |
| 9335 | UGCAGACUUUGUCCAGGGUGA | 15000 | TGCAGACTTTGTCCAGGGTGA |
| 9336 | UGCAGAUCAUUCUCUGGGACA | 15001 | TGCAGATCATTCTCTGGGACA |
| 9337 | UGCAGCCUCAGCCUCUCUUGG | 15002 | TGCAGCCTCAGCCTCTCTTGG |
| 9338 | UGCAGCCUCAGCCUCUGUGAG | 15003 | TGCAGCCTCAGCCTCTGTGAG |
| 9339 | UGCAGCGGUUGACACGGCCCC | 15004 | TGCAGCGGTTGACACGGCCCC |
| 9340 | UGCAGGCCACGUGUCCUCACG | 15005 | TGCAGGCCACGTGTCCTCACG |
| 9341 | UGCAGGCCUUCGUGUGGGGGU | 15006 | TGCAGGCCTTCGTGTGGGGGT |
| 9342 | UGCAGGUGACAGACAAGCACG | 15007 | TGCAGGTGACAGACAAGCACG |
| 9343 | UGCAGGUGGAGCUGUUGCACU | 15008 | TGCAGGTGGAGCTGTTGCACT |
| 9344 | UGCAGGUGUCGGGAUCCUGAC | 15009 | TGCAGGTGTCGGGATCCTGAC |
| 9345 | UGCAGUCAUAUUCCCGGUCAC | 15010 | TGCAGTCATATTCCCGGTCAC |
| 9346 | UGCAGUCCCCGCCGCGGCGAG | 15011 | TGCAGTCCCCGCCGCGGCGAG |
| 9347 | UGCAGUCGGGGCCACCAUCAC | 15012 | TGCAGTCGGGGCCACCATCAC |
| 9348 | UGCAGUGGCGCAAUCUCGGCU | 15013 | TGCAGTGGCGCAATCTCGGCT |
| 9349 | UGCAGUGGCUCGAUCAUGGCU | 15014 | TGCAGTGGCTCGATCATGGCT |
| 9350 | UGCAGUUUCCAUCAGAGCACU | 15015 | TGCAGTTTCCATCAGAGCACT |
| 9351 | UGCAUAGAAGAGGUAAACACG | 15016 | TGCATAGAAGAGGTAAACACG |
| 9352 | UGCAUAGAAGUGGAAGCACUA | 15017 | TGCATAGAAGTGGAAGCACTA |
| 9353 | UGCAUGGGCACUGUCCGAAGC | 15018 | TGCATGGGCACTGTCCGAAGC |
| 9354 | UGCAUGGUUUUUUUUUGUUUU | 15019 | TGCATGGTTTTTTTTTGTTTT |
| 9355 | UGCAUGUUUUAAACACAUACC | 15020 | TGCATGTTTTAAACACATACC |
| 9356 | UGCAUUCGCCGCUGUGACACU | 15021 | TGCATTCGCCGCTGTGACACT |
| 9357 | UGCAUUUCCCGUCUUGGCACU | 15022 | TGCATTTCCCGTCTTGGCACT |
| 9358 | UGCCAAAUGUACACAGUGUAC | 15023 | TGCCAAATGTACACAGTGTAC |
| 9359 | UGCCAAUCCCUUGUGACAUCU | 15024 | TGCCAATCCCTTGTGACATCT |
| 9360 | UGCCACCACGGAUUCAGCCAG | 15025 | TGCCACCACGGATTCAGCCAG |
| 9361 | UGCCACCGUACCCAGCUGAUU | 15026 | TGCCACCGTACCCAGCTGATT |
| 9362 | UGCCAGCAACGUCGCCCAGAG | 15027 | TGCCAGCAACGTCGCCCAGAG |
| 9363 | UGCCAGGCAGUGUCCCGACCC | 15028 | TGCCAGGCAGTGTCCCGACCC |
| 9364 | UGCCAUCGCAGACCCACUUGU | 15029 | TGCCATCGCAGACCCACTTGT |
| 9365 | UGCCAUCUGCUGUUGUGUGUU | 15030 | TGCCATCTGCTGTTGTGTGTT |
| 9366 | UGCCAUGGAUGCAGUUUCCAU | 15031 | TGCCATGGATGCAGTTTCCAT |
| 9367 | UGCCAUUGGGCCACUGAAUGU | 15032 | TGCCATTGGGCCACTGAATGT |
| 9368 | UGCCCAGGACAGAGUCGGUCC | 15033 | TGCCCAGGACAGAGTCGGTCC |
| 9369 | UGCCCAGGCUGGAAAGCAGUG | 15034 | TGCCCAGGCTGGAAAGCAGTG |
| 9370 | UGCCCAGGCUGGAGUGCAGUG | 15035 | TGCCCAGGCTGGAGTGCAGTG |
| 9371 | UGCCCCCGAGACCAGAGACCC | 15036 | TGCCCCCGAGACCAGAGACCC |
| 9372 | UGCCCCCGUUGACAUCGAUGC | 15037 | TGCCCCCGTTGACATCGATGC |
| 9373 | UGCCCCUCUCUGUCCCCGGCA | 15038 | TGCCCCTCTCTGTCCCCGGCA |
| 9374 | UGCCCGCCACCACGCCCGGCU | 15039 | TGCCCGCCACCACGCCCGGCT |
| 9375 | UGCCGAGAGAUGCACUUCCCA | 15040 | TGCCGAGAGATGCACTTCCCA |
| 9376 | UGCCGGCUGCCAUGGAUGCAG | 15041 | TGCCGGCTGCCATGGATGCAG |
| 9377 | UGCCGGUUGGUGAAGAAGAGG | 15042 | TGCCGGTTGGTGAAGAAGAGG |
| 9378 | UGCCGUCCGGGCAGGCGCAGG | 15043 | TGCCGTCCGGGCAGGCGCAGG |
| 9379 | UGCCUCAGCCUCCCAAGUAGC | 15044 | TGCCTCAGCCTCCCAAGTAGC |
| 9380 | UGCCUCAGCCUCCCGAGUAGC | 15045 | TGCCTCAGCCTCCCGAGTAGC |
| 9381 | UGCCUCCCAGAUGAAUAAAUA | 15046 | TGCCTCCCAGATGAATAAATA |
| 9382 | UGCCUCUGGGAAGUGGCAUCA | 15047 | TGCCTCTGGGAAGTGGCATCA |
| 9383 | UGCCUGAGCUCAAACCAUCCU | 15048 | TGCCTGAGCTCAAACCATCCT |
| 9384 | UGCCUGGCCAGAGGCCAUGGG | 15049 | TGCCTGGCCAGAGGCCATGGG |
| 9385 | UGCGCCCGGCCUCACCGUGCA | 15050 | TGCGCCCGGCCTCACCGTGCA |
| 9386 | UGCGGACUGCAGUGGCGCAAU | 15051 | TGCGGACTGCAGTGGCGCAAT |
| 9387 | UGCGGCCACUCAUCCGAGCCA | 15052 | TGCGGCCACTCATCCGAGCCA |
| 9388 | UGCGGGGCAGGGAGGCACAGA | 15053 | TGCGGGGCAGGGAGGCACAGA |
| 9389 | UGCUCAGGCCUGGUCUCCAAC | 15054 | TGCTCAGGCCTGGTCTCCAAC |
| 9390 | UGCUCAGGGUGGUCCUCUCAC | 15055 | TGCTCAGGGTGGTCCTCTCAC |
| 9391 | UGCUCCGAAACCAGAAAGGCU | 15056 | TGCTCCGAAACCAGAAAGGCT |
| 9392 | UGCUCGCAGCCUCUGCCAGGC | 15057 | TGCTCGCAGCCTCTGCCAGGC |
| 9393 | UGCUCGUCUGAGCCGUUGUCG | 15058 | TGCTCGTCTGAGCCGTTGTCG |
| 9394 | UGCUGAUGACGGUGUCAUAGG | 15059 | TGCTGATGACGGTGTCATAGG |
| 9395 | UGCUGAUGCACCAUGCAUGGU | 15060 | TGCTGATGCACCATGCATGGT |
| 9396 | UGCUGCAGAUCAUUCUCUGGG | 15061 | TGCTGCAGATCATTCTCTGGG |
| 9397 | UGCUGGCCACCUCCGUGUCCA | 15062 | TGCTGGCCACCTCCGTGTCCA |
| 9398 | UGCUGGGAUUACAGGCGUGAG | 15063 | TGCTGGGATTACAGGCGTGAG |
| 9399 | UGCUGUGGAUCCAGUCCACAG | 15064 | TGCTGTGGATCCAGTCCACAG |
| 9400 | UGCUGUGUCCUAGCUGGAAAC | 15065 | TGCTGTGTCCTAGCTGGAAAC |
| 9401 | UGCUGUGUCCUUACGGCUGUG | 15066 | TGCTGTGTCCTTACGGCTGTG |
| 9402 | UGCUGUUGAUGUUCUUAAGCC | 15067 | TGCTGTTGATGTTCTTAAGCC |
| 9403 | UGCUGUUGUGUGUUGAAAGCC | 15068 | TGCTGTTGTGTGTTGAAAGCC |
| 9404 | UGCUUCUCUGGGCCUGACAUC | 15069 | TGCTTCTCTGGGCCTGACATC |
| 9405 | UGCUUGAGAUGGAGUGAAGUU | 15070 | TGCTTGAGATGGAGTGAAGTT |
| 9406 | UGCUUUGGUCUUCUCUGUCUU | 15071 | TGCTTTGGTCTTCTCTGTCTT |
| 9407 | UGGAAACCCUGGCUUCCCGCG | 15072 | TGGAAACCCTGGCTTCCCGCG |
| 9408 | UGGAAAGCAGUGGCAUAAUCA | 15073 | TGGAAAGCAGTGGCATAATCA |
| 9409 | UGGAACACGUAAAGACCCCUA | 15074 | TGGAACACGTAAAGACCCCTA |
| 9410 | UGGAACUCGAAGGCCGAGCAG | 15075 | TGGAACTCGAAGGCCGAGCAG |
| 9411 | UGGAACUCGUUUCUUUCGCAU | 15076 | TGGAACTCGTTTCTTTCGCAT |
| 9412 | UGGAAGAGAACCAUAUCCUCU | 15077 | TGGAAGAGAACCATATCCTCT |
| 9413 | UGGAAGCACUAGGUGGGCGGA | 15078 | TGGAAGCACTAGGTGGGCGGA |
| 9414 | UGGAAGCCGUCGGGGCACAGG | 15079 | TGGAAGCCGTCGGGGCACAGG |
| 9415 | UGGAAGCCUUCCUCACACUGG | 15080 | TGGAAGCCTTCCTCACACTGG |
| 9416 | UGGAAGCUGGCGGGACCACAG | 15081 | TGGAAGCTGGCGGGACCACAG |
| 9417 | UGGAAGUGGGUAGGGGUCGGG | 15082 | TGGAAGTGGGTAGGGGTCGGG |
| 9418 | UGGAAUUCGUCAGGGCGACAG | 15083 | TGGAATTCGTCAGGGCGACAG |
| 9419 | UGGACAGAGCCCUCACGCUAC | 15084 | TGGACAGAGCCCTCACGCTAC |
| 9420 | UGGACCUCAUCCUCUGUGGUC | 15085 | TGGACCTCATCCTCTGTGGTC |
| 9421 | UGGAGAGAAACUCAAAACUUC | 15086 | TGGAGAGAAACTCAAAACTTC |
| 9422 | UGGAGAGAAAUGGAGGUGUCA | 15087 | TGGAGAGAAATGGAGGTGTCA |
| 9423 | UGGAGCCCACAGCCUUGCAGG | 15088 | TGGAGCCCACAGCCTTGCAGG |
| 9424 | UGGAGCCGUUCUCCCUGAAUA | 15089 | TGGAGCCGTTCTCCCTGAATA |
| 9425 | UGGAGCUGACCUUUAGCCUGA | 15090 | TGGAGCTGACCTTTAGCCTGA |
| 9426 | UGGAGCUGUUGCACUGGAAGC | 15091 | TGGAGCTGTTGCACTGGAAGC |
| 9427 | UGGAGGAUUUAUUUAUUUUUU | 15092 | TGGAGGATTTATTTATTTTTT |
| 9428 | UGGAGGCAUUUGCAUAGAAGU | 15093 | TGGAGGCATTTGCATAGAAGT |
| 9429 | UGGAGGUGUCAUCCUGGUCCC | 15094 | TGGAGGTGTCATCCTGGTCCC |
| 9430 | UGGAGUCAACCCAGUAGAGGC | 15095 | TGGAGTCAACCCAGTAGAGGC |
| 9431 | UGGAGUGAAGUUUGGAGUCAA | 15096 | TGGAGTGAAGTTTGGAGTCAA |
| 9432 | UGGAGUGCAGUGGCUCGAUCA | 15097 | TGGAGTGCAGTGGCTCGATCA |
| 9433 | UGGAGUGGAUGCACUCGCCAC | 15098 | TGGAGTGGATGCACTCGCCAC |
| 9434 | UGGAUCCAGUCCACAGCCAGC | 15099 | TGGATCCAGTCCACAGCCAGC |
| 9435 | UGGAUGAGGCAGGGCCAGGGG | 15100 | TGGATGAGGCAGGGCCAGGGG |
| 9436 | UGGAUGCACUCGCCACUUAGG | 15101 | TGGATGCACTCGCCACTTAGG |
| 9437 | UGGAUGCAGUUUCCAUCAGAG | 15102 | TGGATGCAGTTTCCATCAGAG |
| 9438 | UGGAUGUCUCCUGGGUGGCCA | 15103 | TGGATGTCTCCTGGGTGGCCA |
| 9439 | UGGAUGUCUCUGCUGAUGACG | 15104 | TGGATGTCTCTGCTGATGACG |
| 9440 | UGGCAAAUGUGGACCUCAUCC | 15105 | TGGCAAATGTGGACCTCATCC |
| 9441 | UGGCACUCAGCGCUGCCAUCG | 15106 | TGGCACTCAGCGCTGCCATCG |
| 9442 | UGGCACUGAAAAUGGCUUCGU | 15107 | TGGCACTGAAAATGGCTTCGT |
| 9443 | UGGCACUGGAACUCGUUUCUU | 15108 | TGGCACTGGAACTCGTTTCTT |
| 9444 | UGGCACUUGUAGCCACCCUCC | 15109 | TGGCACTTGTAGCCACCCTCC |
| 9445 | UGGCAGCCGCCAUUGCUCAGG | 15110 | TGGCAGCCGCCATTGCTCAGG |
| 9446 | UGGCAGCGAAACUCGUCCUGG | 15111 | TGGCAGCGAAACTCGTCCTGG |
| 9447 | UGGCAGGCAAUGCUUUGGUCU | 15112 | TGGCAGGCAATGCTTTGGTCT |
| 9448 | UGGCAGUUCUCGAUAUUCACG | 15113 | TGGCAGTTCTCGATATTCACG |
| 9449 | UGGCAUAAUCAUAGCUCACGA | 15114 | TGGCATAATCATAGCTCACGA |
| 9450 | UGGCAUCAUUUGGUGAAUGAG | 15115 | TGGCATCATTTGGTGAATGAG |
| 9451 | UGGCCAAAUUCAUUUUUUAAG | 15116 | TGGCCAAATTCATTTTTTAAG |
| 9452 | UGGCCACAGCGCAGUUUUCCU | 15117 | TGGCCACAGCGCAGTTTTCCT |
| 9453 | UGGCCACAGGACAGCCUUGCU | 15118 | TGGCCACAGGACAGCCTTGCT |
| 9454 | UGGCCACCUCCGUGUCCAGAG | 15119 | TGGCCACCTCCGTGTCCAGAG |
| 9455 | UGGCCACUGCAGCCUCAGCCU | 15120 | TGGCCACTGCAGCCTCAGCCT |
| 9456 | UGGCCAGAGGCCAUGGGCUGC | 15121 | TGGCCAGAGGCCATGGGCTGC |
| 9457 | UGGCCAGCAGCAUGCCGUCCG | 15122 | TGGCCAGCAGCATGCCGTCCG |
| 9458 | UGGCCAUCGCACCUCCAGAAC | 15123 | TGGCCATCGCACCTCCAGAAC |
| 9459 | UGGCCCCAGGCAGCCGGGAGG | 15124 | TGGCCCCAGGCAGCCGGGAGG |
| 9460 | UGGCCCUUGGCUUGGAGCCGU | 15125 | TGGCCCTTGGCTTGGAGCCGT |
| 9461 | UGGCCGGGGGCCCCUCUGUGA | 15126 | TGGCCGGGGGCCCCTCTGTGA |
| 9462 | UGGCCUGGGAUCCCAUCCCAA | 15127 | TGGCCTGGGATCCCATCCCAA |
| 9463 | UGGCGAUGGGGAGGACAAUGG | 15128 | TGGCGATGGGGAGGACAATGG |
| 9464 | UGGCGCAAUCUCGGCUCACUG | 15129 | TGGCGCAATCTCGGCTCACTG |
| 9465 | UGGCGGGACCACAGGUGAGCA | 15130 | TGGCGGGACCACAGGTGAGCA |
| 9466 | UGGCGGGAGUUCCCCAGUCAG | 15131 | TGGCGGGAGTTCCCCAGTCAG |
| 9467 | UGGCUCACUGCAACCUCCACC | 15132 | TGGCTCACTGCAACCTCCACC |
| 9468 | UGGCUCGAUCAUGGCUCACUG | 15133 | TGGCTCGATCATGGCTCACTG |
| 9469 | UGGCUGAAGAUCUCGGCUGCA | 15134 | TGGCTGAAGATCTCGGCTGCA |
| 9470 | UGGCUGCAGGUGUCGGGAUCC | 15135 | TGGCTGCAGGTGTCGGGATCC |
| 9471 | UGGCUGGGUGAGGUUGUGGAA | 15136 | TGGCTGGGTGAGGTTGTGGAA |
| 9472 | UGGCUGUCUAGCAAGGCUUGC | 15137 | TGGCTGTCTAGCAAGGCTTGC |
| 9473 | UGGCUUAAGGAAGGUACAAAC | 15138 | TGGCTTAAGGAAGGTACAAAC |
| 9474 | UGGCUUAGAGAUUGGUGGAUG | 15139 | TGGCTTAGAGATTGGTGGATG |
| 9475 | UGGCUUCGUUGAUGAUAUCUG | 15140 | TGGCTTCGTTGATGATATCTG |
| 9476 | UGGCUUGGAGCCGUUCUCCCU | 15141 | TGGCTTGGAGCCGTTCTCCCT |
| 9477 | UGGCUUGGAGGCAUUUGCAUA | 15142 | TGGCTTGGAGGCATTTGCATA |
| 9478 | UGGGAACAGCCGCCGUUGUUG | 15143 | TGGGAACAGCCGCCGTTGTTG |
| 9479 | UGGGAAGUGGCAUCAUUUGGU | 15144 | TGGGAAGTGGCATCATTTGGT |
| 9480 | UGGGACAGGUCAGACCAGUAG | 15145 | TGGGACAGGTCAGACCAGTAG |
| 9481 | UGGGACUACAGGUGCCCGCCA | 15146 | TGGGACTACAGGTGCCCGCCA |
| 9482 | UGGGACUCAUCAGAGCCAUCC | 15147 | TGGGACTCATCAGAGCCATCC |
| 9483 | UGGGAGCACGUCUUGGGGGAG | 15148 | TGGGAGCACGTCTTGGGGGAG |
| 9484 | UGGGAGCACGUCUUGGGGGGA | 15149 | TGGGAGCACGTCTTGGGGGGA |
| 9485 | UGGGAUCACAGGCACGUGCCA | 15150 | TGGGATCACAGGCACGTGCCA |
| 9486 | UGGGAUCCCAUCCCAACACAC | 15151 | TGGGATCCCATCCCAACACAC |
| 9487 | UGGGAUCUCGCUUUGUUGCUC | 15152 | TGGGATCTCGCTTTGTTGCTC |
| 9488 | UGGGAUUACAGGCGUGAGCCA | 15153 | TGGGATTACAGGCGTGAGCCA |
| 9489 | UGGGAUUACAGGCGUGAGGCA | 15154 | TGGGATTACAGGCGTGAGGCA |
| 9490 | UGGGCACCAGGCUGGUGUCCU | 15155 | TGGGCACCAGGCTGGTGTCCT |
| 9491 | UGGGCACUGUCCGAAGCCUGU | 15156 | TGGGCACTGTCCGAAGCCTGT |
| 9492 | UGGGCAGGGACGGGACUCCAG | 15157 | TGGGCAGGGACGGGACTCCAG |
| 9493 | UGGGCCACCAGCUGGAAGCCG | 15158 | TGGGCCACCAGCTGGAAGCCG |
| 9494 | UGGGCCACUGAAUGUUUUCAG | 15159 | TGGGCCACTGAATGTTTTCAG |
| 9495 | UGGGCCAGCCUCUUUUCAUCC | 15160 | TGGGCCAGCCTCTTTTCATCC |
| 9496 | UGGGCCUGACAUCCCCUCCCC | 15161 | TGGGCCTGACATCCCCTCCCC |
| 9497 | UGGGCGAGUGGGGGUUGAUCU | 15162 | TGGGCGAGTGGGGGTTGATCT |
| 9498 | UGGGCGGACAUAAAGACGACA | 15163 | TGGGCGGACATAAAGACGACA |
| 9499 | UGGGCUCAGGCGAUCCUCCUG | 15164 | TGGGCTCAGGCGATCCTCCTG |
| 9500 | UGGGCUCUGUCAAGCUGGGUG | 15165 | TGGGCTCTGTCAAGCTGGGTG |
| 9501 | UGGGCUGCUGAUGCACCAUGC | 15166 | TGGGCTGCTGATGCACCATGC |
| 9502 | UGGGCUUCUUCUCAUUUCCUC | 15167 | TGGGCTTCTTCTCATTTCCTC |
| 9503 | UGGGGAAGUGAAUGGCUUGGA | 15168 | TGGGGAAGTGAATGGCTTGGA |
| 9504 | UGGGGACAAUUGCCAAUCCCU | 15169 | TGGGGACAATTGCCAATCCCT |
| 9505 | UGGGGACAGUAGGUUUUCAGC | 15170 | TGGGGACAGTAGGTTTTCAGC |
| 9506 | UGGGGACGGAGGUGUCGCCAC | 15171 | TGGGGACGGAGGTGTCGCCAC |
| 9507 | UGGGGAGGACAAUGGACAGAG | 15172 | TGGGGAGGACAATGGACAGAG |
| 9508 | UGGGGAUGAGGCUGGUGUACU | 15173 | TGGGGATGAGGCTGGTGTACT |
| 9509 | UGGGGCGAGGGCGCCCCCCAG | 15174 | TGGGGCGAGGGCGCCCCCCAG |
| 9510 | UGGGGGAGCACGUCUCCUGGG | 15175 | TGGGGGAGCACGTCTCCTGGG |
| 9511 | UGGGGGAUGCAGGUGGAGCUG | 15176 | TGGGGGATGCAGGTGGAGCTG |
| 9512 | UGGGGGGACAGCCUUGCUCGU | 15177 | TGGGGGGACAGCCTTGCTCGT |
| 9513 | UGGGGGGGACACAGUGUUUUG | 15178 | TGGGGGGGACACAGTGTTTTG |
| 9514 | UGGGGGUCCAGCUGGAAGCCU | 15179 | TGGGGGTCCAGCTGGAAGCCT |
| 9515 | UGGGGGUUGAUCUGCGGGGCA | 15180 | TGGGGGTTGATCTGCGGGGCA |
| 9516 | UGGGGUCUUACUAUGUUGCCC | 15181 | TGGGGTCTTACTATGTTGCCC |
| 9517 | UGGGUAGGGGUCGGGAGGAUG | 15182 | TGGGTAGGGGTCGGGAGGATG |
| 9518 | UGGGUCAUGAUUCGGGGGUGA | 15183 | TGGGTCATGATTCGGGGGTGA |
| 9519 | UGGGUCCCUCGCAGAGUGUCA | 15184 | TGGGTCCCTCGCAGAGTGTCA |
| 9520 | UGGGUGAGGUUGUGGAAGAGA | 15185 | TGGGTGAGGTTGTGGAAGAGA |
| 9521 | UGGGUGCAGGCCACGUGUCCU | 15186 | TGGGTGCAGGCCACGTGTCCT |
| 9522 | UGGGUGCUGCAGAUCAUUCUC | 15187 | TGGGTGCTGCAGATCATTCTC |
| 9523 | UGGGUGGCCACUGCAGCCUCA | 15188 | TGGGTGGCCACTGCAGCCTCA |
| 9524 | UGGGUGGGUCAUGAUUCGGGG | 15189 | TGGGTGGGTCATGATTCGGGG |
| 9525 | UGGGUUCAUCUGACCAGUCCC | 15190 | TGGGTTCATCTGACCAGTCCC |
| 9526 | UGGUAUCCGCAACAGAGACAG | 15191 | TGGTATCCGCAACAGAGACAG |
| 9527 | UGGUCCCCCCGGGCCUGUUUC | 15192 | TGGTCCCCCCGGGCCTGTTTC |
| 9528 | UGGUCCCGCACUCUUUGAUGG | 15193 | TGGTCCCGCACTCTTTGATGG |
| 9529 | UGGUCCUCUCACACCAGUUCA | 15194 | TGGTCCTCTCACACCAGTTCA |
| 9530 | UGGUCUCCAACUCCUGAACUC | 15195 | TGGTCTCCAACTCCTGAACTC |
| 9531 | UGGUCUCGAUCUCCUGACCUC | 15196 | TGGTCTCGATCTCCTGACCTC |
| 9532 | UGGUCUCGGUGCCACCACGGA | 15197 | TGGTCTCGGTGCCACCACGGA |
| 9533 | UGGUCUUCCGGUUGCCCCCGU | 15198 | TGGTCTTCCGGTTGCCCCCGT |
| 9534 | UGGUCUUCUCUGUCUUUGAAU | 15199 | TGGTCTTCTCTGTCTTTGAAT |
| 9535 | UGGUCUUCUGAUAGACGGGGU | 15200 | TGGTCTTCTGATAGACGGGGT |
| 9536 | UGGUGAAGAAGAGGUAGGCGA | 15201 | TGGTGAAGAAGAGGTAGGCGA |
| 9537 | UGGUGAAUGAGUUUGGUCUCG | 15202 | TGGTGAATGAGTTTGGTCTCG |
| 9538 | UGGUGAGACAUUGUCACUAUC | 15203 | TGGTGAGACATTGTCACTATC |
| 9539 | UGGUGAGCCCAGGGGUGGCCC | 15204 | TGGTGAGCCCAGGGGTGGCCC |
| 9540 | UGGUGCCAUCUGCUGUUGUGU | 15205 | TGGTGCCATCTGCTGTTGTGT |
| 9541 | UGGUGGAUGAGGCAGGGCCAG | 15206 | TGGTGGATGAGGCAGGGCCAG |
| 9542 | UGGUGGGGCGAGGGCGCCCCC | 15207 | TGGTGGGGCGAGGGCGCCCCC |
| 9543 | UGGUGUACUCGCUCCGGUCCA | 15208 | TGGTGTACTCGCTCCGGTCCA |
| 9544 | UGGUGUCCUGACAGCCACACC | 15209 | TGGTGTCCTGACAGCCACACC |
| 9545 | UGGUUGUGGCAAAUGUGGACC | 15210 | TGGTTGTGGCAAATGTGGACC |
| 9546 | UGGUUGUGUGCUGUGUCCUUA | 15211 | TGGTTGTGTGCTGTGTCCTTA |
| 9547 | UGGUUUAAAAAGUGACACCCA | 15212 | TGGTTTAAAAAGTGACACCCA |
| 9548 | UGGUUUUUGUAGAGAUGGGAU | 15213 | TGGTTTTTGTAGAGATGGGAT |
| 9549 | UGGUUUUUUUUUGUUUUUUUU | 15214 | TGGTTTTTTTTTGTTTTTTTT |
| 9550 | UGUACAACUCUGAACUGAGAA | 15215 | TGTACAACTCTGAACTGAGAA |
| 9551 | UGUACACAGUGUACAACUCUG | 15216 | TGTACACAGTGTACAACTCTG |
| 9552 | UGUACAGGGACGCAUUUACGU | 15217 | TGTACAGGGACGCATTTACGT |
| 9553 | UGUACAUAAAGAAGAGGCAUU | 15218 | TGTACATAAAGAAGAGGCATT |
| 9554 | UGUACUCGCUCCGGUCCAGCG | 15219 | TGTACTCGCTCCGGTCCAGCG |
| 9555 | UGUAGAGAUGGGAUCUCGCUU | 15220 | TGTAGAGATGGGATCTCGCTT |
| 9556 | UGUAGCCACCCUCCAGGUUCA | 15221 | TGTAGCCACCCTCCAGGTTCA |
| 9557 | UGUAGCCGUCCUGGUUGUGGC | 15222 | TGTAGCCGTCCTGGTTGTGGC |
| 9558 | UGUAGGAGAUGCAUUUCCCGU | 15223 | TGTAGGAGATGCATTTCCCGT |
| 9559 | UGUAGGGAGAGGCACGCUCUG | 15224 | TGTAGGGAGAGGCACGCTCTG |
| 9560 | UGUAUAUAUAUAUAUAUAAGU | 15225 | TGTATATATATATATATAAGT |
| 9561 | UGUAUGUCGCGGAGGCGGCUC | 15226 | TGTATGTCGCGGAGGCGGCTC |
| 9562 | UGUAUUUCUGAAAUAGGGUCU | 15227 | TGTATTTCTGAAATAGGGTCT |
| 9563 | UGUAUUUUUAGUAGAGACGGG | 15228 | TGTATTTTTAGTAGAGACGGG |
| 9564 | UGUCAAAGUUGAUGCUGUUGA | 15229 | TGTCAAAGTTGATGCTGTTGA |
| 9565 | UGUCAAGCUGGGUGCUGCAGA | 15230 | TGTCAAGCTGGGTGCTGCAGA |
| 9566 | UGUCACACAGCUGCCCCCGAG | 15231 | TGTCACACAGCTGCCCCCGAG |
| 9567 | UGUCACAUUAACGCAGCCAAC | 15232 | TGTCACATTAACGCAGCCAAC |
| 9568 | UGUCACGGUGGAGAGAAACUC | 15233 | TGTCACGGTGGAGAGAAACTC |
| 9569 | UGUCACUAUCUCCACCGUGGU | 15234 | TGTCACTATCTCCACCGTGGT |
| 9570 | UGUCACUGUCUCUCCGGACAU | 15235 | TGTCACTGTCTCTCCGGACAT |
| 9571 | UGUCAGACAGCCUUGCUCGUC | 15236 | TGTCAGACAGCCTTGCTCGTC |
| 9572 | UGUCAUAGGAAGAGACGCCGU | 15237 | TGTCATAGGAAGAGACGCCGT |
| 9573 | UGUCAUCCUGGUCCCCCCGGG | 15238 | TGTCATCCTGGTCCCCCCGGG |
| 9574 | UGUCCAAAAUACUUUGUCCUC | 15239 | TGTCCAAAATACTTTGTCCTC |
| 9575 | UGUCCAAGCAUUCGUUGGUCC | 15240 | TGTCCAAGCATTCGTTGGTCC |
| 9576 | UGUCCACACCAUUCAGGCCCC | 15241 | TGTCCACACCATTCAGGCCCC |
| 9577 | UGUCCAGAGCGACCACGUUCC | 15242 | TGTCCAGAGCGACCACGTTCC |
| 9578 | UGUCCAGGGUGAUGCAUUCGC | 15243 | TGTCCAGGGTGATGCATTCGC |
| 9579 | UGUCCCCGGCAUUCAUUGACA | 15244 | TGTCCCCGGCATTCATTGACA |
| 9580 | UGUCCCCUUGGAACACGUAAA | 15245 | TGTCCCCTTGGAACACGTAAA |
| 9581 | UGUCCCGACCCGGAUCACGAC | 15246 | TGTCCCGACCCGGATCACGAC |
| 9582 | UGUCCCUGGCCAGCAGCAUGC | 15247 | TGTCCCTGGCCAGCAGCATGC |
| 9583 | UGUCCCUGGGGACAAUUGCCA | 15248 | TGTCCCTGGGGACAATTGCCA |
| 9584 | UGUCCGAAGCCUGUUCUGCCU | 15249 | TGTCCGAAGCCTGTTCTGCCT |
| 9585 | UGUCCUAGCUGGAAACCCUGG | 15250 | TGTCCTAGCTGGAAACCCTGG |
| 9586 | UGUCCUCAAAGACGGCCAAGG | 15251 | TGTCCTCAAAGACGGCCAAGG |
| 9587 | UGUCCUCACGGCCAAGGUAAC | 15252 | TGTCCTCACGGCCAAGGTAAC |
| 9588 | UGUCCUGACAGCCACACCUGG | 15253 | TGTCCTGACAGCCACACCTGG |
| 9589 | UGUCCUUACGGCUGUGGAGCU | 15254 | TGTCCTTACGGCTGTGGAGCT |
| 9590 | UGUCCUUGCAGUCAUAUUCCC | 15255 | TGTCCTTGCAGTCATATTCCC |
| 9591 | UGUCCUUGCAGUCGGGGCCAC | 15256 | TGTCCTTGCAGTCGGGGCCAC |
| 9592 | UGUCGCAGGCCCACAGCUGGG | 15257 | TGTCGCAGGCCCACAGCTGGG |
| 9593 | UGUCGCAGUCCACUUGGCCAU | 15258 | TGTCGCAGTCCACTTGGCCAT |
| 9594 | UGUCGCCACAGAGCACAGCGG | 15259 | TGTCGCCACAGAGCACAGCGG |
| 9595 | UGUCGCCCACGUGGCUGAAGA | 15260 | TGTCGCCCACGTGGCTGAAGA |
| 9596 | UGUCGCCCACUGCAGUCCCCG | 15261 | TGTCGCCCACTGCAGTCCCCG |
| 9597 | UGUCGCCCAGGCCAGACUGCG | 15262 | TGTCGCCCAGGCCAGACTGCG |
| 9598 | UGUCGCGGAGGCGGCUCCGGG | 15263 | TGTCGCGGAGGCGGCTCCGGG |
| 9599 | UGUCGGGAACAGGUCGGGUGG | 15264 | TGTCGGGAACAGGTCGGGTGG |
| 9600 | UGUCGGGAUCCUGACACUCAU | 15265 | TGTCGGGATCCTGACACTCAT |
| 9601 | UGUCGUCGCCUUUGUGACAGG | 15266 | TGTCGTCGCCTTTGTGACAGG |
| 9602 | UGUCUAGCAAGGCUUGCAUAG | 15267 | TGTCTAGCAAGGCTTGCATAG |
| 9603 | UGUCUCAGGCACUUAAUAAAU | 15268 | TGTCTCAGGCACTTAATAAAT |
| 9604 | UGUCUCCUGGGUGGCCACUGC | 15269 | TGTCTCCTGGGTGGCCACTGC |
| 9605 | UGUCUCGAGGGGUAGCUGUAG | 15270 | TGTCTCGAGGGGTAGCTGTAG |
| 9606 | UGUCUCUCCGGACAUCAGUGC | 15271 | TGTCTCTCCGGACATCAGTGC |
| 9607 | UGUCUCUGCUGAUGACGGUGU | 15272 | TGTCTCTGCTGATGACGGTGT |
| 9608 | UGUCUGGCCAAAUUCAUUUUU | 15273 | TGTCTGGCCAAATTCATTTTT |
| 9609 | UGUCUUUGAAUAAAACAAGGC | 15274 | TGTCTTTGAATAAAACAAGGC |
| 9610 | UGUGAACUUGAUCGGGGCUGG | 15275 | TGTGAACTTGATCGGGGCTGG |
| 9611 | UGUGACACUUGAACUUGUUGG | 15276 | TGTGACACTTGAACTTGTTGG |
| 9612 | UGUGACAGGAACCGCGGAGGA | 15277 | TGTGACAGGAACCGCGGAGGA |
| 9613 | UGUGACAUCUUCACGCGGGAG | 15278 | TGTGACATCTTCACGCGGGAG |
| 9614 | UGUGAGGCAGCUCCUCAUGUC | 15279 | TGTGAGGCAGCTCCTCATGTC |
| 9615 | UGUGAGGCGGUUGGCACUGAA | 15280 | TGTGAGGCGGTTGGCACTGAA |
| 9616 | UGUGCUGUGUCCUUACGGCUG | 15281 | TGTGCTGTGTCCTTACGGCTG |
| 9617 | UGUGGAAGAGAACCAUAUCCU | 15282 | TGTGGAAGAGAACCATATCCT |
| 9618 | UGUGGACCUCAUCCUCUGUGG | 15283 | TGTGGACCTCATCCTCTGTGG |
| 9619 | UGUGGAGCUGACCUUUAGCCU | 15284 | TGTGGAGCTGACCTTTAGCCT |
| 9620 | UGUGGAUCCAGUCCACAGCCA | 15285 | TGTGGATCCAGTCCACAGCCA |
| 9621 | UGUGGCAAAUGUGGACCUCAU | 15286 | TGTGGCAAATGTGGACCTCAT |
| 9622 | UGUGGGGGUCCAGCUGGAAGC | 15287 | TGTGGGGGTCCAGCTGGAAGC |
| 9623 | UGUGGUCUUCUGAUAGACGGG | 15288 | TGTGGTCTTCTGATAGACGGG |
| 9624 | UGUGUAUAUAUAUAUAUAUAA | 15289 | TGTGTATATATATATATATAA |
| 9625 | UGUGUCACGGUGGAGAGAAAC | 15290 | TGTGTCACGGTGGAGAGAAAC |
| 9626 | UGUGUCCUAGCUGGAAACCCU | 15291 | TGTGTCCTAGCTGGAAACCCT |
| 9627 | UGUGUCCUUACGGCUGUGGAG | 15292 | TGTGTCCTTACGGCTGTGGAG |
| 9628 | UGUGUCUGGCCAAAUUCAUUU | 15293 | TGTGTCTGGCCAAATTCATTT |
| 9629 | UGUGUGCUGUGUCCUUACGGC | 15294 | TGTGTGCTGTGTCCTTACGGC |
| 9630 | UGUGUGUAUAUAUAUAUAUAU | 15295 | TGTGTGTATATATATATATAT |
| 9631 | UGUGUGUUGAAAGCCACUUGC | 15296 | TGTGTGTTGAAAGCCACTTGC |
| 9632 | UGUGUUGAAAGCCACUUGCUU | 15297 | TGTGTTGAAAGCCACTTGCTT |
| 9633 | UGUUAGCCAGGAUGGUCUCGA | 15298 | TGTTAGCCAGGATGGTCTCGA |
| 9634 | UGUUCACGCCACGUCAUCCUC | 15299 | TGTTCACGCCACGTCATCCTC |
| 9635 | UGUUCUGCCUCCCAGAUGAAU | 15300 | TGTTCTGCCTCCCAGATGAAT |
| 9636 | UGUUCUUAAGCCGCCAGUUCU | 15301 | TGTTCTTAAGCCGCCAGTTCT |
| 9637 | UGUUGAAAGCCACUUGCUUCU | 15302 | TGTTGAAAGCCACTTGCTTCT |
| 9638 | UGUUGAUGUUCUUAAGCCGCC | 15303 | TGTTGATGTTCTTAAGCCGCC |
| 9639 | UGUUGCACUGGAAGCUGGCGG | 15304 | TGTTGCACTGGAAGCTGGCGG |
| 9640 | UGUUGCAGACUUUGUCCAGGG | 15305 | TGTTGCAGACTTTGTCCAGGG |
| 9641 | UGUUGCCCAGGCUGGAAAGCA | 15306 | TGTTGCCCAGGCTGGAAAGCA |
| 9642 | UGUUGCCCAGGCUGGAGUGCA | 15307 | TGTTGCCCAGGCTGGAGTGCA |
| 9643 | UGUUGCUCAGGCCUGGUCUCC | 15308 | TGTTGCTCAGGCCTGGTCTCC |
| 9644 | UGUUGCUGUGGAUCCAGUCCA | 15309 | TGTTGCTGTGGATCCAGTCCA |
| 9645 | UGUUGGGUCCCUCGCAGAGUG | 15310 | TGTTGGGTCCCTCGCAGAGTG |
| 9646 | UGUUGUCCAAGCAUUCGUUGG | 15311 | TGTTGTCCAAGCATTCGTTGG |
| 9647 | UGUUGUGUGUUGAAAGCCACU | 15312 | TGTTGTGTGTTGAAAGCCACT |
| 9648 | UGUUUCUCUUAUCCUUCACGA | 15313 | TGTTTCTCTTATCCTTCACGA |
| 9649 | UGUUUGAGGAUUGUGUCACGG | 15314 | TGTTTGAGGATTGTGTCACGG |
| 9650 | UGUUUUAAACACAUACCCAUC | 15315 | TGTTTTAAACACATACCCATC |
| 9651 | UGUUUUCAGUCACCAGCGAGU | 15316 | TGTTTTCAGTCACCAGCGAGT |
| 9652 | UGUUUUGUCCCUGGGGACAAU | 15317 | TGTTTTGTCCCTGGGGACAAT |
| 9653 | UGUUUUUUUUUUUUGAGACGG | 15318 | TGTTTTTTTTTTTTGAGACGG |
| 9654 | UUAAAAAGUGACACCCAUCUC | 15319 | TTAAAAAGTGACACCCATCTC |
| 9655 | UUAAACACAUACCCAUCAACG | 15320 | TTAAACACATACCCATCAACG |
| 9656 | UUAACGCAGCCAACUUCAUCG | 15321 | TTAACGCAGCCAACTTCATCG |
| 9657 | UUAACUUUUUGGUUUUUGUAG | 15322 | TTAACTTTTTGGTTTTTGTAG |
| 9658 | UUAAGAAAUUUAAUUGAUCAC | 15323 | TTAAGAAATTTAATTGATCAC |
| 9659 | UUAAGAGAUGGGGUCUUACUA | 15324 | TTAAGAGATGGGGTCTTACTA |
| 9660 | UUAAGCCGCCAGUUCUUCCAU | 15325 | TTAAGCCGCCAGTTCTTCCAT |
| 9661 | UUAAGGAAGGUACAAACCCAC | 15326 | TTAAGGAAGGTACAAACCCAC |
| 9662 | UUAAGGGUGACCAGUGACUCA | 15327 | TTAAGGGTGACCAGTGACTCA |
| 9663 | UUAAGGUCAUUGCAGACGUGG | 15328 | TTAAGGTCATTGCAGACGTGG |
| 9664 | UUAAUAAAUAUUAAGGGUGAC | 15329 | TTAATAAATATTAAGGGTGAC |
| 9665 | UUAAUUGAUCACAGUAAGACA | 15330 | TTAATTGATCACAGTAAGACA |
| 9666 | UUACAGGCGUGAGCCACUGCG | 15331 | TTACAGGCGTGAGCCACTGCG |
| 9667 | UUACAGGCGUGAGGCACCUGU | 15332 | TTACAGGCGTGAGGCACCTGT |
| 9668 | UUACGGCUGUGGAGCUGACCU | 15333 | TTACGGCTGTGGAGCTGACCT |
| 9669 | UUACGUGCUCCGAAACCAGAA | 15334 | TTACGTGCTCCGAAACCAGAA |
| 9670 | UUACUAUGUUGCCCAGGCUGG | 15335 | TTACTATGTTGCCCAGGCTGG |
| 9671 | UUAGAGAUUGGUGGAUGAGGC | 15336 | TTAGAGATTGGTGGATGAGGC |
| 9672 | UUAGCCAGGAUGGUCUCGAUC | 15337 | TTAGCCAGGATGGTCTCGATC |
| 9673 | UUAGCCUGACGGUGGAUGUCU | 15338 | TTAGCCTGACGGTGGATGTCT |
| 9674 | UUAGGCAGUGGAACUCGAAGG | 15339 | TTAGGCAGTGGAACTCGAAGG |
| 9675 | UUAGGGGUUUGGCUUAGAGAU | 15340 | TTAGGGGTTTGGCTTAGAGAT |
| 9676 | UUAGUAGAGACGGGGUUUCAC | 15341 | TTAGTAGAGACGGGGTTTCAC |
| 9677 | UUAUAGUUGUAUUUCUGAAAU | 15342 | TTATAGTTGTATTTCTGAAAT |
| 9678 | UUAUAUAUAUGUGUGUAUAUA | 15343 | TTATATATATGTGTGTATATA |
| 9679 | UUAUCACGGUUCCCUGCACUG | 15344 | TTATCACGGTTCCCTGCACTG |
| 9680 | UUAUCCUUCACGAGGAAAGGA | 15345 | TTATCCTTCACGAGGAAAGGA |
| 9681 | UUAUUUAUUUUUUUAUAGUUG | 15346 | TTATTTATTTTTTTATAGTTG |
| 9682 | UUAUUUUUUUAUAGUUGUAUU | 15347 | TTATTTTTTTATAGTTGTATT |
| 9683 | UUCAACCAAUAUACAAAGUGC | 15348 | TTCAACCAATATACAAAGTGC |
| 9684 | UUCAACCUCGUAAGGAACCGA | 15349 | TTCAACCTCGTAAGGAACCGA |
| 9685 | UUCACACCAUUCUCCUGCCUC | 15350 | TTCACACCATTCTCCTGCCTC |
| 9686 | UUCACGAGGAAAGGAAGAAAC | 15351 | TTCACGAGGAAAGGAAGAAAC |
| 9687 | UUCACGAUCACGGCUUUGAAG | 15352 | TTCACGATCACGGCTTTGAAG |
| 9688 | UUCACGCAGAGCUGGCUGCAG | 15353 | TTCACGCAGAGCTGGCTGCAG |
| 9689 | UUCACGCCACGUCAUCCUCCA | 15354 | TTCACGCCACGTCATCCTCCA |
| 9690 | UUCACGCCCUUGGUAUCCGCA | 15355 | TTCACGCCCTTGGTATCCGCA |
| 9691 | UUCACGCGGGAGUCUGACGGA | 15356 | TTCACGCGGGAGTCTGACGGA |
| 9692 | UUCACGUGUUAGCCAGGAUGG | 15357 | TTCACGTGTTAGCCAGGATGG |
| 9693 | UUCACUCCUCUUGGCUGGGUG | 15358 | TTCACTCCTCTTGGCTGGGTG |
| 9694 | UUCAGAAAGAACCUGAAGUCC | 15359 | TTCAGAAAGAACCTGAAGTCC |
| 9695 | UUCAGAGGCAAUAACCCCCUA | 15360 | TTCAGAGGCAATAACCCCCTA |
| 9696 | UUCAGCCAACAAGUUGACAUC | 15361 | TTCAGCCAACAAGTIGACATC |
| 9697 | UUCAGCCAGAUCAUUUCCGAC | 15362 | TTCAGCCAGATCATTTCCGAC |
| 9698 | UUCAGGCCCCCUUUCUUGAUC | 15363 | TTCAGGCCCCCTTTCTTGATC |
| 9699 | UUCAGGCUACAAAAACCUUCU | 15364 | TTCAGGCTACAAAAACCTTCT |
| 9700 | UUCAGUCACCAGCGAGUAGAU | 15365 | TTCAGTCACCAGCGAGTAGAT |
| 9701 | UUCAUCCUCCAAGAUGGUCUU | 15366 | TTCATCCTCCAAGATGGTCTT |
| 9702 | UUCAUCGCUCAUGUCCUUGCA | 15367 | TTCATCGCTCATGTCCTTGCA |
| 9703 | UUCAUCUGACCAGUCCCGGCA | 15368 | TTCATCTGACCAGTCCCGGCA |
| 9704 | UUCAUCUGUUGCCCAGGCUGG | 15369 | TTCATCTGTTGCCCAGGCTGG |
| 9705 | UUCAUCUUCCAUGUUUGAGGA | 15370 | TTCATCTTCCATGTTTGAGGA |
| 9706 | UUCAUUGACACGGGCUUUCCC | 15371 | TTCATTGACACGGGCTTTCCC |
| 9707 | UUCAUUUAAGAAAUUUAAUUG | 15372 | TTCATTTAAGAAATTTAATTG |
| 9708 | UUCAUUUUUUAAGAGAUGGGG | 15373 | TTCATTTTTTAAGAGATGGGG |
| 9709 | UUCCAGCCCCAGGGCCCCAUG | 15374 | TTCCAGCCCCAGGGCCCCATG |
| 9710 | UUCCAUAGAAGGAAGACCCCC | 15375 | TTCCATAGAAGGAAGACCCCC |
| 9711 | UUCCAUCAGAGCACUGGAAUU | 15376 | TTCCATCAGAGCACTGGAATT |
| 9712 | UUCCAUGUUUGAGGAUUGUGU | 15377 | TTCCATGTTTGAGGATTGTGT |
| 9713 | UUCCCAUCGUGGCAGCGAAAC | 15378 | TTCCCATCGTGGCAGCGAAAC |
| 9714 | UUCCCCAGUCAGUCCAGUACA | 15379 | TTCCCCAGTCAGTCCAGTACA |
| 9715 | UUCCCGGGUUCACACCAUUCU | 15380 | TTCCCGGGTTCACACCATTCT |
| 9716 | UUCCCGGUCACACUGCCGGCU | 15381 | TTCCCGGTCACACTGCCGGCT |
| 9717 | UUCCCGUCUUGGCACUGGAAC | 15382 | TTCCCGTCTTGGCACTGGAAC |
| 9718 | UUCCCUGCACUGGGGGGGACA | 15383 | TTCCCTGCACTGGGGGGGACA |
| 9719 | UUCCCUGGCCUGGGAUCCCAU | 15384 | TTCCCTGGCCTGGGATCCCAT |
| 9720 | UUCCGACGCCAUGAAUCCCUU | 15385 | TTCCGACGCCATGAATCCCTT |
| 9721 | UUCCGGUUGCCCCCGUUGACA | 15386 | TTCCGGTTGCCCCCGTTGACA |
| 9722 | UUCCUCACACUGGCACUUGUA | 15387 | TTCCTCACACTGGCACTTGTA |
| 9723 | UUCCUCAGGUUGGGGAUGAGG | 15388 | TTCCTCAGGTTGGGGATGAGG |
| 9724 | UUCCUCGUCAGAUUUGUCCUU | 15389 | TTCCTCGTCAGATTTGTCCTT |
| 9725 | UUCCUCUGCCAGCAACGUCGC | 15390 | TTCCTCTGCCAGCAACGTCGC |
| 9726 | UUCCUCUUCACGCCCUUGGUA | 15391 | TTCCTCTTCACGCCCTTGGTA |
| 9727 | UUCCUGACCUCGUGCCGGUUG | 15392 | TTCCTGACCTCGTGCCGGTTG |
| 9728 | UUCCUGGAGAGAAAUGGAGGU | 15393 | TTCCTGGAGAGAAATGGAGGT |
| 9729 | UUCCUGGCUUAAGGAAGGUAC | 15394 | TTCCTGGCTTAAGGAAGGTAC |
| 9730 | UUCGCAGUCGGGGUCGUUGUC | 15395 | TTCGCAGTCGGGGTCGTTGTC |
| 9731 | UUCGCAUCUGUCGCCCACGUG | 15396 | TTCGCATCTGTCGCCCACGTG |
| 9732 | UUCGCAUCUGUCGCCCACUGC | 15397 | TTCGCATCTGTCGCCCACTGC |
| 9733 | UUCGCAUCUUCGCUGGGCCAC | 15398 | TTCGCATCTTCGCTGGGCCAC |
| 9734 | UUCGCCAGGAGGGCCCAGGAU | 15399 | TTCGCCAGGAGGGCCCAGGAT |
| 9735 | UUCGCCGCUGUGACACUUGAA | 15400 | TTCGCCGCTGTGACACTTGAA |
| 9736 | UUCGCUGGGCCACCAGCUGGA | 15401 | TTCGCTGGGCCACCAGCTGGA |
| 9737 | UUCGGGGGUGACAGGGCAAAG | 15402 | TTCGGGGGTGACAGGGCAAAG |
| 9738 | UUCGUCAGGGCGACAGGUGGC | 15403 | TTCGTCAGGGCGACAGGTGGC |
| 9739 | UUCGUGCAAAUAAUCUUUGUA | 15404 | TTCGTGCAAATAATCTTTGTA |
| 9740 | UUCGUGUGGGGGUCCAGCUGG | 15405 | TTCGTGTGGGGGTCCAGCTGG |
| 9741 | UUCGUUGAUGAUAUCUGUCCA | 15406 | TTCGTTGATGATATCTGTCCA |
| 9742 | UUCGUUGGUCCCGCACUCUUU | 15407 | TTCGTTGGTCCCGCACTCTTT |
| 9743 | UUCUAUACAGUGGUUUAAAAA | 15408 | TTCTATACAGTGGTTTAAAAA |
| 9744 | UUCUAUUGCUGGCCACCUCCG | 15409 | TTCTATTGCTGGCCACCTCCG |
| 9745 | UUCUCAUUUCCUCUGCCAGCA | 15410 | TTCTCATTTCCTCTGCCAGCA |
| 9746 | UUCUCCCAAAAAGCGUUGCAC | 15411 | TTCTCCCAAAAAGCGTTGCAC |
| 9747 | UUCUCCCUGAAUAACGUUUUC | 15412 | TTCTCCCTGAATAACGTTTTC |
| 9748 | UUCUCCUGCCUCAGCCUCCCG | 15413 | TTCTCCTGCCTCAGCCTCCCG |
| 9749 | UUCUCGAUAUUCACGAUCACG | 15414 | TTCTCGATATTCACGATCACG |
| 9750 | UUCUCUGGGACAGGUCAGACC | 15415 | TTCTCTGGGACAGGTCAGACC |
| 9751 | UUCUCUGGGCCUGACAUCCCC | 15416 | TTCTCTGGGCCTGACATCCCC |
| 9752 | UUCUCUGUCUUUGAAUAAAAC | 15417 | TTCTCTGTCTTTGAATAAAAC |
| 9753 | UUCUCUUAUCCUUCACGAGGA | 15418 | TTCTCTTATCCTTCACGAGGA |
| 9754 | UUCUGAAAUAGGGUCUUCAUC | 15419 | TTCTGAAATAGGGTCTTCATC |
| 9755 | UUCUGAGGCAACGUUUCAACC | 15420 | TTCTGAGGCAACGTTTCAACC |
| 9756 | UUCUGAUAGACGGGGUUGUCA | 15421 | TTCTGATAGACGGGGTTGTCA |
| 9757 | UUCUGCCUCCCAGAUGAAUAA | 15422 | TTCTGCCTCCCAGATGAATAA |
| 9758 | UUCUGGGCAGGGACGGGACUC | 15423 | TTCTGGGCAGGGACGGGACTC |
| 9759 | UUCUUAAGCCGCCAGUUCUUC | 15424 | TTCTTAAGCCGCCAGTTCTTC |
| 9760 | UUCUUCCAUAGAAGGAAGACC | 15425 | TTCTTCCATAGAAGGAAGACC |
| 9761 | UUCUUCUCAUUUCCUCUGCCA | 15426 | TTCTTCTCATTTCCTCTGCCA |
| 9762 | UUCUUGAUCUUGGCGGGAGUU | 15427 | TTCTTGATCTTGGCGGGAGTT |
| 9763 | UUCUUUCGCAUCUGUCGCCCA | 15428 | TTCTTTCGCATCTGTCGCCCA |
| 9764 | UUGAAAGCCACUUGCUUCUCU | 15429 | TTGAAAGCCACTTGCTTCTCT |
| 9765 | UUGAACUUGUUGGGUCCCUCG | 15430 | TTGAACTTGTTGGGTCCCTCG |
| 9766 | UUGAAGUCCCGGUCAACCUGC | 15431 | TTGAAGTCCCGGTCAACCTGC |
| 9767 | UUGAAUAAAACAAGGCCGGCG | 15432 | TTGAATAAAACAAGGCCGGCG |
| 9768 | UUGACACGGCCCCCACAGCUG | 15433 | TTGACACGGCCCCCACAGCTG |
| 9769 | UUGACACGGGCUUUCCCUGGC | 15434 | TTGACACGGGCTTTCCCTGGC |
| 9770 | UUGACAUCGAUGCUUGAGAUG | 15435 | TTGACATCGATGCTTGAGATG |
| 9771 | UUGACAUCGGAACCUGUGAGG | 15436 | TTGACATCGGAACCTGTGAGG |
| 9772 | UUGACUCCUGAGUUUAGGGGU | 15437 | TTGACTCCTGAGTTTAGGGGT |
| 9773 | UUGAGACGGAGUCUCGCUCUG | 15438 | TTGAGACGGAGTCTCGCTCTG |
| 9774 | UUGAGAUGGAGUGAAGUUUGG | 15439 | TTGAGATGGAGTGAAGTTTGG |
| 9775 | UUGAGGAUUGUGUCACGGUGG | 15440 | TTGAGGATTGTGTCACGGTGG |
| 9776 | UUGAGGGUGGCCGGGGGCCCC | 15441 | TTGAGGGTGGCCGGGGGCCCC |
| 9777 | UUGAUCACAGUAAGACAUUCA | 15442 | TTGATCACAGTAAGACATTCA |
| 9778 | UUGAUCGGGGCUGGUGGGGCG | 15443 | TTGATCGGGGCTGGTGGGGCG |
| 9779 | UUGAUCUGCGGGGCAGGGAGG | 15444 | TTGATCTGCGGGGCAGGGAGG |
| 9780 | UUGAUCUUGGCGGGAGUUCCC | 15445 | TTGATCTTGGCGGGAGTTCCC |
| 9781 | UUGAUGAUAUCUGUCCAAAAU | 15446 | TTGATGATATCTGTCCAAAAT |
| 9782 | UUGAUGCUGUUGAUGUUCUUA | 15447 | TTGATGCTGTTGATGTTCTTA |
| 9783 | UUGAUGGGUUCAUCUGACCAG | 15448 | TTGATGGGTTCATCTGACCAG |
| 9784 | UUGAUGUUCUUAAGCCGCCAG | 15449 | TTGATGTTCTTAAGCCGCCAG |
| 9785 | UUGCAAAAAUAAAUAGAUUUU | 15450 | TTGCAAAAATAAATAGATTTT |
| 9786 | UUGCACACAGUCCAGUUCGUG | 15451 | TTGCACACAGTCCAGTTCGTG |
| 9787 | UUGCACUGGAAGCUGGCGGGA | 15452 | TTGCACTGGAAGCTGGCGGGA |
| 9788 | UUGCAGACGUGGGAACAGCCG | 15453 | TTGCAGACGTGGGAACAGCCG |
| 9789 | UUGCAGACUUUGUCCAGGGUG | 15454 | TTGCAGACTTTGTCCAGGGTG |
| 9790 | UUGCAGGCCUUCGUGUGGGGG | 15455 | TTGCAGGCCTTCGTGTGGGGG |
| 9791 | UUGCAGGUGACAGACAAGCAC | 15456 | TTGCAGGTGACAGACAAGCAC |
| 9792 | UUGCAGUCAUAUUCCCGGUCA | 15457 | TTGCAGTCATATTCCCGGTCA |
| 9793 | UUGCAGUCGGGGCCACCAUCA | 15458 | TTGCAGTCGGGGCCACCATCA |
| 9794 | UUGCAUAGAAGAGGUAAACAC | 15459 | TTGCATAGAAGAGGTAAACAC |
| 9795 | UUGCAUAGAAGUGGAAGCACU | 15460 | TTGCATAGAAGTGGAAGCACT |
| 9796 | UUGCAUGGGCACUGUCCGAAG | 15461 | TTGCATGGGCACTGTCCGAAG |
| 9797 | UUGCCAAUCCCUUGUGACAUC | 15462 | TTGCCAATCCCTTGTGACATC |
| 9798 | UUGCCCAGGCUGGAAAGCAGU | 15463 | TTGCCCAGGCTGGAAAGCAGT |
| 9799 | UUGCCCAGGCUGGAGUGCAGU | 15464 | TTGCCCAGGCTGGAGTGCAGT |
| 9800 | UUGCCCCCGUUGACAUCGAUG | 15465 | TTGCCCCCGTTGACATCGATG |
| 9801 | UUGCUCAGGCCUGGUCUCCAA | 15466 | TTGCTCAGGCCTGGTCTCCAA |
| 9802 | UUGCUCAGGGUGGUCCUCUCA | 15467 | TTGCTCAGGGTGGTCCTCTCA |
| 9803 | UUGCUCGUCUGAGCCGUUGUC | 15468 | TTGCTCGTCTGAGCCGTTGTC |
| 9804 | UUGCUGGCCACCUCCGUGUCC | 15469 | TTGCTGGCCACCTCCGTGTCC |
| 9805 | UUGCUGUGGAUCCAGUCCACA | 15470 | TTGCTGTGGATCCAGTCCACA |
| 9806 | UUGCUUCUCUGGGCCUGACAU | 15471 | TTGCTTCTCTGGGCCTGACAT |
| 9807 | UUGGAACACGUAAAGACCCCU | 15472 | TTGGAACACGTAAAGACCCCT |
| 9808 | UUGGAGCCGUUCUCCCUGAAU | 15473 | TTGGAGCCGTTCTCCCTGAAT |
| 9809 | UUGGAGGCAUUUGCAUAGAAG | 15474 | TTGGAGGCATTTGCATAGAAG |
| 9810 | UUGGAGUCAACCCAGUAGAGG | 15475 | TTGGAGTCAACCCAGTAGAGG |
| 9811 | UUGGCACUGAAAAUGGCUUCG | 15476 | TTGGCACTGAAAATGGCTTCG |
| 9812 | UUGGCACUGGAACUCGUUUCU | 15477 | TTGGCACTGGAACTCGTTTCT |
| 9813 | UUGGCCAUCGCACCUCCAGAA | 15478 | TTGGCCATCGCACCTCCAGAA |
| 9814 | UUGGCGGGAGUUCCCCAGUCA | 15479 | TTGGCGGGAGTTCCCCAGTCA |
| 9815 | UUGGCUGGGUGAGGUUGUGGA | 15480 | TTGGCTGGGTGAGGTTGTGGA |
| 9816 | UUGGCUUAGAGAUUGGUGGAU | 15481 | TTGGCTTAGAGATTGGTGGAT |
| 9817 | UUGGCUUGGAGCCGUUCUCCC | 15482 | TTGGCTTGGAGCCGTTCTCCC |
| 9818 | UUGGGCCACUGAAUGUUUUCA | 15483 | TTGGGCCACTGAATGTTTTCA |
| 9819 | UUGGGCGAGUGGGGGUUGAUC | 15484 | TTGGGCGAGTGGGGGTTGATC |
| 9820 | UUGGGGAAGUGAAUGGCUUGG | 15485 | TTGGGGAAGTGAATGGCTTGG |
| 9821 | UUGGGGAUGAGGCUGGUGUAC | 15486 | TTGGGGATGAGGCTGGTGTAC |
| 9822 | UUGGGGGAGCACGUCUCCUGG | 15487 | TTGGGGGAGCACGTCTCCTGG |
| 9823 | UUGGGGGGACAGCCUUGCUCG | 15488 | TTGGGGGGACAGCCTTGCTCG |
| 9824 | UUGGGUCCCUCGCAGAGUGUC | 15489 | TTGGGTCCCTCGCAGAGTGTC |
| 9825 | UUGGUAUCCGCAACAGAGACA | 15490 | TTGGTATCCGCAACAGAGACA |
| 9826 | UUGGUCCCGCACUCUUUGAUG | 15491 | TTGGTCCCGCACTCTTTGATG |
| 9827 | UUGGUCUCGGUGCCACCACGG | 15492 | TTGGTCTCGGTGCCACCACGG |
| 9828 | UUGGUCUUCUCUGUCUUUGAA | 15493 | TTGGTCTTCTCTGTCTTTGAA |
| 9829 | UUGGUGAAGAAGAGGUAGGCG | 15494 | TTGGTGAAGAAGAGGTAGGCG |
| 9830 | UUGGUGAAUGAGUUUGGUCUC | 15495 | TTGGTGAATGAGTTTGGTCTC |
| 9831 | UUGGUGAGACAUUGUCACUAU | 15496 | TTGGTGAGACATTGTCACTAT |
| 9832 | UUGGUGCCAUCUGCUGUUGUG | 15497 | TTGGTGCCATCTGCTGTTGTG |
| 9833 | UUGGUGGAUGAGGCAGGGCCA | 15498 | TTGGTGGATGAGGCAGGGCCA |
| 9834 | UUGGUUUUUGUAGAGAUGGGA | 15499 | TTGGTTTTTGTAGAGATGGGA |
| 9835 | UUGUACAUAAAGAAGAGGCAU | 15500 | TTGTACATAAAGAAGAGGCAT |
| 9836 | UUGUAGAGAUGGGAUCUCGCU | 15501 | TTGTAGAGATGGGATCTCGCT |
| 9837 | UUGUAGCCACCCUCCAGGUUC | 15502 | TTGTAGCCACCCTCCAGGTTC |
| 9838 | UUGUAGGAGAUGCAUUUCCCG | 15503 | TTGTAGGAGATGCATTTCCCG |
| 9839 | UUGUAUUUCUGAAAUAGGGUC | 15504 | TTGTATTTCTGAAATAGGGTC |
| 9840 | UUGUAUUUUUAGUAGAGACGG | 15505 | TTGTATTTTTAGTAGAGACGG |
| 9841 | UUGUCAAAGUUGAUGCUGUUG | 15506 | TTGTCAAAGTTGATGCTGTTG |
| 9842 | UUGUCACUAUCUCCACCGUGG | 15507 | TTGTCACTATCTCCACCGTGG |
| 9843 | UUGUCCAAGCAUUCGUUGGUC | 15508 | TTGTCCAAGCATTCGTTGGTC |
| 9844 | UUGUCCAGGGUGAUGCAUUCG | 15509 | TTGTCCAGGGTGATGCATTCG |
| 9845 | UUGUCCCUGGGGACAAUUGCC | 15510 | TTGTCCCTGGGGACAATTGCC |
| 9846 | UUGUCCUCAAAGACGGCCAAG | 15511 | TTGTCCTCAAAGACGGCCAAG |
| 9847 | UUGUCCUUGCAGUCGGGGCCA | 15512 | TTGTCCTTGCAGTCGGGGCCA |
| 9848 | UUGUCGCAGGCCCACAGCUGG | 15513 | TTGTCGCAGGCCCACAGCTGG |
| 9849 | UUGUCGCAGUCCACUUGGCCA | 15514 | TTGTCGCAGTCCACTTGGCCA |
| 9850 | UUGUCGUCGCCUUUGUGACAG | 15515 | TTGTCGTCGCCTTTGTGACAG |
| 9851 | UUGUGACAGGAACCGCGGAGG | 15516 | TTGTGACAGGAACCGCGGAGG |
| 9852 | UUGUGACAUCUUCACGCGGGA | 15517 | TTGTGACATCTTCACGCGGGA |
| 9853 | UUGUGGAAGAGAACCAUAUCC | 15518 | TTGTGGAAGAGAACCATATCC |
| 9854 | UUGUGGCAAAUGUGGACCUCA | 15519 | TTGTGGCAAATGTGGACCTCA |
| 9855 | UUGUGUCACGGUGGAGAGAAA | 15520 | TTGTGTCACGGTGGAGAGAAA |
| 9856 | UUGUGUGCUGUGUCCUUACGG | 15521 | TTGTGTGCTGTGTCCTTACGG |
| 9857 | UUGUGUGUUGAAAGCCACUUG | 15522 | TTGTGTGTTGAAAGCCACTTG |
| 9858 | UUGUUGCUCAGGCCUGGUCUC | 15523 | TTGTTGCTCAGGCCTGGTCTC |
| 9859 | UUGUUGGGUCCCUCGCAGAGU | 15524 | TTGTTGGGTCCCTCGCAGAGT |
| 9860 | UUGUUGUCCAAGCAUUCGUUG | 15525 | TTGTTGTCCAAGCATTCGTTG |
| 9861 | UUGUUUUUUUUUUUUGAGACG | 15526 | TTGTTTTTTTTTTTTGAGACG |
| 9862 | UUUAAAAAGUGACACCCAUCU | 15527 | TTTAAAAAGTGACACCCATCT |
| 9863 | UUUAAACACAUACCCAUCAAC | 15528 | TTTAAACACATACCCATCAAC |
| 9864 | UUUAACUUUUUGGUUUUUGUA | 15529 | TTTAACTTTTTGGTTTTTGTA |
| 9865 | UUUAAGAAAUUUAAUUGAUCA | 15530 | TTTAAGAAATTTAATTGATCA |
| 9866 | UUUAAGAGAUGGGGUCUUACU | 15531 | TTTAAGAGATGGGGTCTTACT |
| 9867 | UUUAAUUGAUCACAGUAAGAC | 15532 | TTTAATTGATCACAGTAAGAC |
| 9868 | UUUACGUGCUCCGAAACCAGA | 15533 | TTTACGTGCTCCGAAACCAGA |
| 9869 | UUUAGCCUGACGGUGGAUGUC | 15534 | TTTAGCCTGACGGTGGATGTC |
| 9870 | UUUAGGGGUUUGGCUUAGAGA | 15535 | TTTAGGGGTTTGGCTTAGAGA |
| 9871 | UUUAGUAGAGACGGGGUUUCA | 15536 | TTTAGTAGAGACGGGGTTTCA |
| 9872 | UUUAUAGUUGUAUUUCUGAAA | 15537 | TTTATAGTTGTATTTCTGAAA |
| 9873 | UUUAUAUAUAUGUGUGUAUAU | 15538 | TTTATATATATGTGTGTATAT |
| 9874 | UUUAUUUAUUUUUUUAUAGUU | 15539 | TTTATTTATTTTTTTATAGTT |
| 9875 | UUUAUUUUUUUAUAGUUGUAU | 15540 | TTTATTTTTTTATAGTTGTAT |
| 9876 | UUUCAACCAAUAUACAAAGUG | 15541 | TTTCAACCAATATACAAAGTG |
| 9877 | UUUCAACCUCGUAAGGAACCG | 15542 | TTTCAACCTCGTAAGGAACCG |
| 9878 | UUUCACGUGUUAGCCAGGAUG | 15543 | TTTCACGTGTTAGCCAGGATG |
| 9879 | UUUCAGAAAGAACCUGAAGUC | 15544 | TTTCAGAAAGAACCTGAAGTC |
| 9880 | UUUCAGAGGCAAUAACCCCCU | 15545 | TTTCAGAGGCAATAACCCCCT |
| 9881 | UUUCAGCCAACAAGUUGACAU | 15546 | TTTCAGCCAACAAGTTGACAT |
| 9882 | UUUCAGUCACCAGCGAGUAGA | 15547 | TTTCAGTCACCAGCGAGTAGA |
| 9883 | UUUCAUCCUCCAAGAUGGUCU | 15548 | TTTCATCCTCCAAGATGGTCT |
| 9884 | UUUCAUCUUCCAUGUUUGAGG | 15549 | TTTCATCTTCCATGTTTGAGG |
| 9885 | UUUCCAGCCCCAGGGCCCCAU | 15550 | TTTCCAGCCCCAGGGCCCCAT |
| 9886 | UUUCCAUCAGAGCACUGGAAU | 15551 | TTTCCATCAGAGCACTGGAAT |
| 9887 | UUUCCCGUCUUGGCACUGGAA | 15552 | TTTCCCGTCTTGGCACTGGAA |
| 9888 | UUUCCCUGGCCUGGGAUCCCA | 15553 | TTTCCCTGGCCTGGGATCCCA |
| 9889 | UUUCCGACGCCAUGAAUCCCU | 15554 | TTTCCGACGCCATGAATCCCT |
| 9890 | UUUCCUCGUCAGAUUUGUCCU | 15555 | TTTCCTCGTCAGATTTGTCCT |
| 9891 | UUUCCUCUGCCAGCAACGUCG | 15556 | TTTCCTCTGCCAGCAACGTCG |
| 9892 | UUUCCUCUUCACGCCCUUGGU | 15557 | TTTCCTCTTCACGCCCTTGGT |
| 9893 | UUUCCUGGCUUAAGGAAGGUA | 15558 | TTTCCTGGCTTAAGGAAGGTA |
| 9894 | UUUCGCAUCUGUCGCCCACGU | 15559 | TTTCGCATCTGTCGCCCACGT |
| 9895 | UUUCGCAUCUGUCGCCCACUG | 15560 | TTTCGCATCTGTCGCCCACTG |
| 9896 | UUUCUCUUAUCCUUCACGAGG | 15561 | TTTCTCTTATCCTTCACGAGG |
| 9897 | UUUCUGAAAUAGGGUCUUCAU | 15562 | TTTCTGAAATAGGGTCTTCAT |
| 9898 | UUUCUUGAUCUUGGCGGGAGU | 15563 | TTTCTTGATCTTGGCGGGAGT |
| 9899 | UUUCUUUCGCAUCUGUCGCCC | 15564 | TTTCTTTCGCATCTGTCGCCC |
| 9900 | UUUGAAGUCCCGGUCAACCUG | 15565 | TTTGAAGTCCCGGTCAACCTG |
| 9901 | UUUGAAUAAAACAAGGCCGGC | 15566 | TTTGAATAAAACAAGGCCGGC |
| 9902 | UUUGAGACGGAGUCUCGCUCU | 15567 | TTTGAGACGGAGTCTCGCTCT |
| 9903 | UUUGAGGAUUGUGUCACGGUG | 15568 | TTTGAGGATTGTGTCACGGTG |
| 9904 | UUUGAUGGGUUCAUCUGACCA | 15569 | TTTGATGGGTTCATCTGACCA |
| 9905 | UUUGCAAAAAUAAAUAGAUUU | 15570 | TTTGCAAAAATAAATAGATTT |
| 9906 | UUUGCAGGUGACAGACAAGCA | 15571 | TTTGCAGGTGACAGACAAGCA |
| 9907 | UUUGCAUAGAAGUGGAAGCAC | 15572 | TTTGCATAGAAGTGGAAGCAC |
| 9908 | UUUGGAGUCAACCCAGUAGAG | 15573 | TTTGGAGTCAACCCAGTAGAG |
| 9909 | UUUGGCUUAGAGAUUGGUGGA | 15574 | TTTGGCTTAGAGATTGGTGGA |
| 9910 | UUUGGUCUCGGUGCCACCACG | 15575 | TTTGGTCTCGGTGCCACCACG |
| 9911 | UUUGGUCUUCUCUGUCUUUGA | 15576 | TTTGGTCTTCTCTGTCTTTGA |
| 9912 | UUUGGUGAAUGAGUUUGGUCU | 15577 | TTTGGTGAATGAGTTTGGTCT |
| 9913 | UUUGGUUUUUGUAGAGAUGGG | 15578 | TTTGGTTTTTGTAGAGATGGG |
| 9914 | UUUGUACAUAAAGAAGAGGCA | 15579 | TTTGTACATAAAGAAGAGGCA |
| 9915 | UUUGUAGAGAUGGGAUCUCGC | 15580 | TTTGTAGAGATGGGATCTCGC |
| 9916 | UUUGUAUUUUUAGUAGAGACG | 15581 | TTTGTATTTTTAGTAGAGACG |
| 9917 | UUUGUCCAGGGUGAUGCAUUC | 15582 | TTTGTCCAGGGTGATGCATTC |
| 9918 | UUUGUCCCUGGGGACAAUUGC | 15583 | TTTGTCCCTGGGGACAATTGC |
| 9919 | UUUGUCCUCAAAGACGGCCAA | 15584 | TTTGTCCTCAAAGACGGCCAA |
| 9920 | UUUGUCCUUGCAGUCGGGGCC | 15585 | TTTGTCCTTGCAGTCGGGGCC |
| 9921 | UUUGUGACAGGAACCGCGGAG | 15586 | TTTGTGACAGGAACCGCGGAG |
| 9922 | UUUGUUGCUCAGGCCUGGUCU | 15587 | TTTGTTGCTCAGGCCTGGTCT |
| 9923 | UUUGUUUUUUUUUUUUGAGAC | 15588 | TTTGTTTTTTTTTTTTGAGAC |
| 9924 | UUUUAAACACAUACCCAUCAA | 15589 | TTTTAAACACATACCCATCAA |
| 9925 | UUUUAACUUUUUGGUUUUUGU | 15590 | TTTTAACTTTTTGGTTTTTGT |
| 9926 | UUUUAAGAGAUGGGGUCUUAC | 15591 | TTTTAAGAGATGGGGTCTTAC |
| 9927 | UUUUAGUAGAGACGGGGUUUC | 15592 | TTTTAGTAGAGACGGGGTTTC |
| 9928 | UUUUAUAGUUGUAUUUCUGAA | 15593 | TTTTATAGTTGTATTTCTGAA |
| 9929 | UUUUAUAUAUAUGUGUGUAUA | 15594 | TTTTATATATATGTGTGTATA |
| 9930 | UUUUCAGCCAACAAGUUGACA | 15595 | TTTTCAGCCAACAAGTTGACA |
| 9931 | UUUUCAGUCACCAGCGAGUAG | 15596 | TTTTCAGTCACCAGCGAGTAG |
| 9932 | UUUUCAUCCUCCAAGAUGGUC | 15597 | TTTTCATCCTCCAAGATGGTC |
| 9933 | UUUUCCUCGUCAGAUUUGUCC | 15598 | TTTTCCTCGTCAGATTTGTCC |
| 9934 | UUUUCCUCUUCACGCCCUUGG | 15599 | TTTTCCTCTTCACGCCCTTGG |
| 9935 | UUUUGAGACGGAGUCUCGCUC | 15600 | TTTTGAGACGGAGTCTCGCTC |
| 9936 | UUUUGGUUUUUGUAGAGAUGG | 15601 | TTTTGGTTTTTGTAGAGATGG |
| 9937 | UUUUGUAGAGAUGGGAUCUCG | 15602 | TTTTGTAGAGATGGGATCTCG |
| 9938 | UUUUGUAUUUUUAGUAGAGAC | 15603 | TTTTGTATTTTTAGTAGAGAC |
| 9939 | UUUUGUCCCUGGGGACAAUUG | 15604 | TTTTGTCCCTGGGGACAATTG |
| 9940 | UUUUGUUUUUUUUUUUUGAGA | 15605 | TTTTGTTTTTTTTTTTTGAGA |
| 9941 | UUUUUAACUUUUUGGUUUUUG | 15606 | TTTTTAACTTTTTGGTTTTTG |
| 9942 | UUUUUAAGAGAUGGGGUCUUA | 15607 | TTTTTAAGAGATGGGGTCTTA |
| 9943 | UUUUUAGUAGAGACGGGGUUU | 15608 | TTTTTAGTAGAGACGGGGTTT |
| 9944 | UUUUUAUAGUUGUAUUUCUGA | 15609 | TTTTTATAGTTGTATTTCTGA |
| 9945 | UUUUUGAGACGGAGUCUCGCU | 15610 | TTTTTGAGACGGAGTCTCGCT |
| 9946 | UUUUUGGUUUUUGUAGAGAUG | 15611 | TTTTTGGTTTTTGTAGAGATG |
| 9947 | UUUUUGUAGAGAUGGGAUCUC | 15612 | TTTTTGTAGAGATGGGATCTC |
| 9948 | UUUUUGUAUUUUUAGUAGAGA | 15613 | TTTTTGTATTTTTAGTAGAGA |
| 9949 | UUUUUGUUUUUUUUUUUUGAG | 15614 | TTTTTGTTTTTTTTTTTTGAG |
| 9950 | UUUUUUAAGAGAUGGGGUCUU | 15615 | TTTTTTAAGAGATGGGGTCTT |
| 9951 | UUUUUUAUAGUUGUAUUUCUG | 15616 | TTTTTTATAGTTGTATTTCTG |
| 9952 | UUUUUUGAGACGGAGUCUCGC | 15617 | TTTTTTGAGACGGAGTCTCGC |
| 9953 | UUUUUUGUAUUUUUAGUAGAG | 15618 | TTTTTTGTATTTTTAGTAGAG |
| 9954 | UUUUUUGUUUUUUUUUUUUGA | 15619 | TTTTTTGTTTTTTTTTTTTGA |
| 9955 | UUUUUUUAUAGUUGUAUUUCU | 15620 | TTTTTTTATAGTTGTATTTCT |
| 9956 | UUUUUUUGAGACGGAGUCUCG | 15621 | TTTTTTTGAGACGGAGTCTCG |
| 9957 | UUUUUUUGUUUUUUUUUUUUG | 15622 | TTTTTTTGTTTTTTTTTTTTG |
| 9958 | UUUUUUUUGAGACGGAGUCUC | 15623 | TTTTTTTTGAGACGGAGTCTC |
| 9959 | UUUUUUUUGUUUUUUUUUUUU | 15624 | TTTTTTTTGTTTTTTTTTTTT |
| 9960 | UUUUUUUUUGAGACGGAGUCU | 15625 | TTTTTTTTTGAGACGGAGTCT |
| 9961 | UUUUUUUUUGUUUUUUUUUUU | 15626 | TTTTTTTTTGTTTTTTTTTTT |
| 9962 | UUUUUUUUUUGAGACGGAGUC | 15627 | TTTTTTTTTTGAGACGGAGTC |
| 9963 | UUUUUUUUUUUGAGACGGAGU | 15628 | TTTTTTTTTTTGAGACGGAGT |
| 9964 | UUUUUUUUUUUUGAGACGGAG | 15629 | TTTTTTTTTTTTGAGACGGAG |

What is claimed is:

1. A composition comprising an agent that blocks low-density lipoprotein (LDL) binding to a low-density lipoprotein receptor (LDLR) and a delivery molecule, wherein the delivery molecule is capable of delivering the agent to the liver.

2. The composition of claim 1, further comprising a payload.

3. The composition of claim 2, wherein the payload comprises a therapeutic agent.

4. The composition of claim 2 or 3, wherein the payload comprises a component of a CRISPR/Cas system or a nucleic acid encoding one or more component of a CRISPR/Cas system, a biologic, or a small molecule, optionally wherein the component of a CRISPR/Cas system comprises a nucleic acid encoding one or more guide RNAs, one or more scaffolds, and/or one or more endonucleases.

5. The composition of any one of claims 2-4, further comprising a lipid nanoparticle (LNP) or lipoplex, wherein the LNP or lipoplex encapsulates the payload.

6. The composition of any one of claims 1-5, wherein the delivery molecule comprises a lipid nanoparticle (LNP) or lipoplex.

7. The composition of claim 6, wherein the delivery molecule comprises an LNP comprising LP-01, cholesterol, DSPC, and/or DMG-PEG2000.

8. The composition of claim 7, wherein the LNP comprises about 50% LP-01, about 39% cholesterol, about 9% DSPC, and about 2% DMG-PEG2000.

9. The composition of any one of claims 1-8, wherein the delivery molecule comprises one or more of the following: lactose, galactose, N-acetylgalactosamine (GalNAc), GalNAc-6, galactosamine, N-formylgalactosamine, N-acetylgalactosamine, N-propionylgalactosamine, N-butanoylgalactosamine, N-isobutanoyl-galactosamine, and cholesterol, or a derivative thereof.

10. The composition of claim 9, wherein the delivery molecule comprises N-acetylgalactosamine (GalNAc).

11. The composition of claim 10, wherein the delivery molecule comprises GalNAc-6.

12. The composition of any one of claims 1-11, wherein the agent comprises an RNAi.

13. The composition of claim 12, wherein the RNAi is a small-interfering RNA (siRNA), an antisense oligonucleotide (ASO), microRNA (miRNA), double-stranded RNA (dsRNA), short hairpin RNA (shRNA) or an expression cassette encoding an RNA.

14. The composition of claim 12, wherein the agent comprises an siRNA.

15. The composition of claim 14, wherein the siRNA binds to a nucleic acid comprising the nucleotide sequence of any one of SEQ ID NOs: 350-352, or the reverse complement thereof.

16. The composition of claim 14, wherein the siRNA comprises at least 13, at least 14, at least 15, at least 16, at least 17, at least 18, at least 19, at least 20, or at least 21 consecutive nucleic acids of any one of the nucleic acid sequences in Table 3B.

17. The composition of claim 14, wherein the siRNA is at least 80%, at least 85%, at least 90%, or at least 95% identical to any one of the nucleic acid sequences in Table 3B.

18. The composition of claim 12, wherein the agent comprises an antisense oligonucleotide (ASO).

19. The composition of any one of claims 1-18, wherein the LDLR comprises an amino acid sequence that is at least 85%, 90%, 91%, 92%, 93%, 94%, 95%, 96%, 97%, 98%, 99% or 100% identical to any one of the sequences of SEQ ID NOs: 353-355.

20. The composition of claim 4, wherein the component of the CRISPR/Cas system comprises a nucleic acid encoding one or more guide RNAs and a Cas9, wherein the nucleic acid molecule comprises:
a. a first nucleic acid encoding one or more guide RNAs selected from any one of SEQ ID NOs: 1-35, 1000-1078, or 3000-3069 and a second nucleic acid encoding a Staphylococcus aureus Cas9 (SaCas9); or
b. a first nucleic acid encoding one or more guide RNAs selected from any one of SEQ ID NOs: 100-225, 2000-2116, or 4000-4251 and a second nucleic acid encoding a Staphylococcus lugdunensis (SluCas9); or
c. a first nucleic acid encoding one or more guide RNAs comprising at least 20 contiguous nucleotides of a guide RNA selected from any one of SEQ ID NOs: 1-35, 1000-1078, or 3000-3069 and a second nucleic acid encoding a Staphylococcus aureus Cas9 (SaCas9); or
d. a first nucleic acid encoding one or more guide RNAs comprising at least 20 contiguous nucleotides of a guide RNA selected from any one of SEQ ID NOs: 100-225, 2000-2116, or 4000-4251 and a second nucleic acid encoding a Staphylococcus lugdunensis (SluCas9); or
e. a first nucleic acid encoding one or more guide RNAs that is at least 90% identical to any one of SEQ ID NOs: 1-35, 1000-1078, or 3000-3069 and a second nucleic acid encoding a Staphylococcus aureus Cas9 (SaCas9); or
f. a first nucleic acid encoding one or more guide RNAs that is at least 90% identical to any one of SEQ ID NOs: 100-225, 2000-2116, or 4000-4251 and a second nucleic acid encoding a Staphylococcus lugdunensis (SluCas9); or
g. a first nucleic acid encoding a pair of guide RNAs comprising a first and second guide RNA selected from any one of the following pairs of guide RNAs: SEQ ID NOs: 1020 and 23; 1023 and 23; 1023 and 1037; 1024 and 1055; 1025 and 23; 1025 and 1055; 1026 and 23; 1028 and 1055; 1029 and 1055; 1029 and 1037; 1031 and 1037; 1032 and 1037; 101029 and 1027; 1037 and 1048; 1037 and 1051; 1037 and 1053; 20 and 23; 1038 and 23; 21 and 23; 1040 and 23; 1042 and 1037; 1043 and 1037; 1044 and 1037; 1045 and 1037; 1046 and 1037; 24 and 1037; 1047 and 1055; or 1055 and 1022; and a second nucleic acid encoding a Staphylococcus aureus Cas9 (SaCas9); or
h. a first nucleic acid encoding a pair of guide RNAs comprising a first and second guide RNA selected from any one of the following pairs of guide RNAs: SEQ ID NOs: 170 and 179; 172 and 179; 179 and 183; 179 and 185; 179 and 187; 179 and 188; 179 and 189; 179 and 193; 179 and 195; 179 and 196; 179 and 197; 200 and 174; or 200 and 176; and a second nucleic acid encoding a Staphylococcus lugdunensis (SluCas9);
i. a first nucleic acid encoding a pair of guide RNAs comprising a first and second guide RNA selected from any one of the following pairs of guide RNAs: SEQ ID NOs: 117 and 121; 117 and 122; 120 and 121; 120 and 123; 120 and 124; 120 and 125; 122 and 126; 122 and 123; 122 and 124; 122 and 125; or 122 and 126; and a second nucleic acid encoding a Staphylococcus lugdunensis (SluCas9); for targeting exon 44;
j. a first nucleic acid encoding a pair of guide RNAs comprising a first and second guide RNA selected from any one of the following pairs of guide RNAs: 155 and 156; 155 and 158; 155 and 162; 155 and 163; 162 and 157; 162 and 159; 162 and 164; 162 and 166; or 162 and 167; and a second nucleic acid encoding a Staphylococcus lugdunensis (SluCas9); for targeting exon 50;
k. a first nucleic acid encoding a pair of guide RNAs comprising a first and second guide RNA selected from any one of the following pairs of guide RNAs: SEQ ID NOs: 211 and 223; 211 and 225; 214 and 224; 216 and 223; 216 and 225; 220 and 224; 204 and 223; 223 and 224; or 204 and 225; and a second nucleic acid encoding a Staphylococcus lugdunensis (SluCas9); for targeting exon 53;
l. a first nucleic acid encoding a pair of guide RNAs comprising a first and second guide RNA selected from any one of the following pairs of guide RNAs: SEQ ID NOs: 1068 and 32; 1069 and 32; 1070 and 1075; 1071 and 32; 29 and 1075; 1072 and 27; 1072 and 28; 1072 and 32; 1072 and 33; 1073 and 1076; 1073 and 35; 221 and 1077; 1074 and 27; 1074 and 28; 1074 and 33; 32 and 1077; 1075 and 1076; 1075 and 35; 1076 and 26; or 35 and 26; and a second nucleic acid encoding a Staphylococcus aureus Cas9 (SaCas9); for targeting exon 53;
m. a first nucleic acid encoding a pair of guide RNAs comprising a first and second guide RNA selected from any one of the following pairs of guide RNAs: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
n. a first nucleic acid encoding a pair of guide RNAs comprising a first and second guide RNA selected from any one of the following pairs of guide RNAs: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9).

21. A method comprising (a) administering to a subject in need thereof an agent that blocks LDL binding to a low-density lipoprotein receptor (LDLR) and concurrently or sequentially (b) administering an LNP or lipoplex comprising a payload to the subject, wherein the agent reduces off-target delivery of the LNP or lipoplex to the liver.

22. A method of increasing the percentage of payload delivered to a non-liver target in a subject, comprising (a) a pre-conditioning step comprising administering to the subject a composition comprising an agent that blocks LDL binding to a low-density lipoprotein receptor (LDLR) in the liver, and then (b) administering an LNP or lipoplex and a payload to the subject.

23. The method of any one of claims 21-22, wherein step (a) comprises administering to the subject the composition of any one of claims 1, and 6-20.

24. A method of decreasing liver tropism of a payload administered in an LNP or lipoplex in a subject comprising (a) administering to the subject a composition of any one of claims 1, and 6-20, and then (b) administering an LNP or lipoplex and a payload to the subject.

25. The method of any of claims 21-24, wherein administering to the subject a composition comprising an agent that blocks LDL binding to a low-density lipoprotein receptor (LDLR) in the liver temporarily blocks the delivery molecule from interacting with the LDLR in the liver.

26. The method of any of claims 21-25, wherein administering to the subject the composition and/or agent that blocks LDL binding to a low-density lipoprotein receptor (LDLR) in the liver occurs about 2 days, about 3 days, about 4 days, about 5 days, about 6 days, about 7 days, about 8 days, about 9 days, about 10 days, about 11 days, about 12 days, about 13 days, about 14 days, about 15 days, about 16, days, about 17 days, about 18 days, about 19 days, about 20 days, or about 21 days prior to administering the LNP or lipoplex, and payload.

27. The method of any one of claims 21-25, wherein administering to the subject the composition and/or agent that blocks LDL binding to a low-density lipoprotein receptor (LDLR) in the liver occurs about 1 day, about 2 days, about 3 days, about 4 days, about 5 days, about 6 days, about 7 days prior to administering the LNP or lipoplex and payload.

28. The method of any of claims 21-25, wherein administering to the subject the composition and/or agent that blocks LDL binding to a low-density lipoprotein receptor (LDLR) in the liver occurs about 8 days, about 9 days, about 10 days, about 11 days, about 12 days, about 13 days, or about 14 days prior to administering the LNP or lipoplex and payload.

29. The method of any of claims 21-25 wherein administering to the subject the composition of any one of claims 1, and 6-20 immediately precedes administering the LNP or lipoplex and payload.

30. The method of any of claims 21-25, wherein the composition of any one of claims 1, and 6-20 and the LNP or lipoplex and payload are co-administered.

31. The method of any of claims 21-30, wherein the delivery molecule is a lipid nanoparticle (LNP).

32. The method of any of claims 21-31, wherein the payload is a component of a CRISPR/Cas system or a nucleic acid encoding one or more components thereof, a biologic, or a small molecule, optionally wherein the component of a CRISPR/Cas system comprises a nucleic acid encoding one or more guide RNAs, one or more scaffolds, and/or one or more endonucleases.

33. The method of claim 32, wherein the component of a CRISPR/Cas system comprises a nucleic acid encoding one or more guide RNAs and a Cas9, wherein the nucleic acid molecule comprises:
a. a first nucleic acid encoding one or more guide RNAs selected from any one of SEQ ID NOs: 1-35, 1000-1078, or 3000-3069 and a second nucleic acid encoding a Staphylococcus aureus Cas9 (SaCas9); or
b. a first nucleic acid encoding one or more guide RNAs selected from any one of SEQ ID NOs: 100-225, 2000-2116, or 4000-4251 and a second nucleic acid encoding a Staphylococcus lugdunensis (SluCas9); or
c. a first nucleic acid encoding one or more guide RNAs comprising at least 20 contiguous nucleotides of a guide RNA selected from any one of SEQ ID NOs: 1-35, 1000-1078, or 3000-3069 and a second nucleic acid encoding a Staphylococcus aureus Cas9 (SaCas9); or
d. a first nucleic acid encoding one or more guide RNAs comprising at least 20 contiguous nucleotides of a guide RNA selected from any one of SEQ ID NOs: 100-225, 2000-2116, or 4000-4251 and a second nucleic acid encoding a Staphylococcus lugdunensis (SluCas9); or
e. a first nucleic acid encoding one or more guide RNAs that is at least 90% identical to any one of SEQ ID NOs: 1-35, 1000-1078, or 3000-3069 and a second nucleic acid encoding a Staphylococcus aureus Cas9 (SaCas9); or
f. a first nucleic acid encoding one or more guide RNAs that is at least 90% identical to any one of SEQ ID NOs: 100-225, 2000-2116, or 4000-4251 and a second nucleic acid encoding a Staphylococcus lugdunensis (SluCas9); or
g. a first nucleic acid encoding a pair of guide RNAs comprising a first and second guide RNA selected from any one of the following pairs of guide RNAs: SEQ ID NOs: 1020 and 23; 1023 and 23; 1023 and 1037; 1024 and 1055; 1025 and 23; 1025 and 1055; 1026 and 23; 1028 and 1055; 1029 and 1055; 1029 and 1037; 1031 and 1037; 1032 and 1037; 101029 and 1027; 1037 and 1048; 1037 and 1051; 1037 and 1053; 20 and 23; 1038 and 23; 21 and 23; 1040 and 23; 1042 and 1037; 1043 and 1037; 1044 and 1037; 1045 and 1037; 1046 and 1037; 24 and 1037; 1047 and 1055; or 1055 and 1022; and a second nucleic acid encoding a Staphylococcus aureus Cas9 (SaCas9); or
h. a first nucleic acid encoding a pair of guide RNAs comprising a first and second guide RNA selected from any one of the following pairs of guide RNAs: SEQ ID NOs: 170 and 179; 172 and 179; 179 and 183; 179 and 185; 179 and 187; 179 and 188; 179 and 189; 179 and 193; 179 and 195; 179 and 196; 179 and 197; 200 and 174; or 200 and 176; and a second nucleic acid encoding a Staphylococcus lugdunensis (SluCas9);
i. a first nucleic acid encoding a pair of guide RNAs comprising a first and second guide RNA selected from any one of the following pairs of guide RNAs: SEQ ID NOs: 117 and 121; 117 and 122; 120 and 121; 120 and 123; 120 and 124; 120 and 125; 122 and 126; 122 and 123; 122 and 124; 122 and 125; or 122 and 126; and a second nucleic acid encoding a Staphylococcus lugdunensis (SluCas9); for targeting exon 44;
j. a first nucleic acid encoding a pair of guide RNAs comprising a first and second guide RNA selected from any one of the following pairs of guide RNAs: 155 and 156; 155 and 158; 155 and 162; 155 and 163; 162 and 157; 162 and 159; 162 and 164; 162 and 166; or 162 and 167; and a second nucleic acid encoding a Staphylococcus lugdunensis (SluCas9); for targeting exon 50;
k. a first nucleic acid encoding a pair of guide RNAs comprising a first and second guide RNA selected from any one of the following pairs of guide RNAs: SEQ ID NOs: 211 and 223; 211 and 225; 214 and 224; 216 and 223; 216 and 225; 220 and 224; 204 and 223; 223 and 224; or 204 and 225; and a second nucleic acid encoding a Staphylococcus lugdunensis (SluCas9); for targeting exon 53;
l. a first nucleic acid encoding a pair of guide RNAs comprising a first and second guide RNA selected from any one of the following pairs of guide RNAs: SEQ ID NOs: 1068 and 32; 1069 and 32; 1070 and 1075; 1071 and 32; 29 and 1075; 1072 and 27; 1072 and 28; 1072 and 32; 1072 and 33; 1073 and 1076; 1073 and 35; 221 and 1077; 1074 and 27; 1074 and 28; 1074 and 33; 32 and 1077; 1075 and 1076; 1075 and 35; 1076 and 26; or 35 and 26; and a second nucleic acid encoding a Staphylococcus aureus Cas9 (SaCas9); for targeting exon 53;
m. a first nucleic acid encoding a pair of guide RNAs comprising a first and second guide RNA selected from any one of the following pairs of guide RNAs: SEQ ID NOs: 148 and 134; 149 and 135; 150 and 135; 131 and 136; 151 and 136; 139 and 131; 139 and 151; 140 and 131; 140 and 151; 141 and 148; 144 and 149; 144 and 150; 145 and 131; 145 and 151; 146 and 148; 134 and 148; 135 and 149; 135 and 150; 136 and 131; 136 and 151; 131 and 139; 151 and 139; 131 and 140; 151 and 140; 148 and 141; 149 and 144; 150 and 144; 131 and 145; 151 and 145; and 148 and 146; and a second nucleic acid encoding a Staphylococcus lugdunensis (SluCas9); or
n. a first nucleic acid encoding a pair of guide RNAs comprising a first and second guide RNA selected from any one of the following pairs of guide RNAs: SEQ ID NOs: 10 and 15; 10 and 16; 12 and 16; 1001 and 1005; 1001 and 15; 1001 and 16; 1003 and 1005; 16 and 1003; 12 and 1010; 12 and 1012; 12 and 1013; 10 and 1016; 1017 and 1005; 1017 and 16; 1018 and 16; 15 and 10; 16 and 10; 16 and 12; 1005 and 1001; 15 and 1001; 16 and 1001; 1005 and 1003; 1003 and 16; 1010 and 12; 1012 and 12; 1013 and 12; 1016 and 10; 1005 and 1017; 16 and 1017; and 16 and 1018; and a second nucleic acid encoding a Staphylococcus aureus Cas9 (SaCas9).

34. The method of any one of claims 21-33, wherein the payload is encapsulated within a lipid nanoparticle (LNP).

35. The method of any of claims 21-34, wherein the method comprises co-administering other drugs that facilitate increased uptake of the agent in the liver.

36. The method of any of any of claims 21-35 wherein the blocking of LDL to a low-density lipoprotein receptor (LDLR) in the liver in step (a) is not temporary.

37. The method of any one of claims 21-36, wherein the payload is within an LNP, and the LNP targets the brain; spinal cord; eye; retina; bone; cardiac muscle, skeletal muscle, smooth muscle; lung; pancreas; heart; and/or kidney.

38. The method of any one of claims 21-37, wherein administering the composition in step (a) increases the percentage of payload delivered to a non-liver target.

39. The method of any one of claims 21-38, wherein the method results in at least a 10%, 30%, 50%, 75%, 100%, 125%, 150%, 200%, 250%, 300%, 350%, 400%, 450%, 500%, 600%, 700%, 800%, 900%, or 1000% increase of payload in a non-liver target as compared to the payload in the corresponding tissue of a control subject that received the payload but did not receive the agent that blocks LDL binding to a low-density lipoprotein receptor (LDLR) in the liver.

40. The method of claim 39, wherein the method results in at least a 10%, 30%, 50%, 75%, 100%, 125%, 150%, 200%, 250%, 300%, 350%, 400%, 450%, 500%, 600%, 700%, 800%, 900%, or 1000% increase of payload in skeletal muscle as compared to the payload in the corresponding muscle of a control subject that received the payload but did not receive the agent that blocks LDL binding to a low-density lipoprotein receptor (LDLR) in the liver.

41. The method of any one of claims 21-40, wherein the agent comprises an RNAi.

42. The method of claim 41, wherein the RNAi is a small-interfering RNA (siRNA), an antisense oligonucleotide (ASO), microRNA (miRNA), double-stranded RNA (dsRNA), short hairpin RNA (shRNA) or an expression cassette encoding an RNA.

43. The method of any one of claims 21-40, wherein the agent comprises an siRNA.

44. The method of claim 43, wherein the siRNA binds to a nucleic acid comprising the nucleotide sequence of any one of SEQ ID NOs: 350-352, or the reverse complement thereof.

45. The method of claim 43, wherein the siRNA comprises at least 13, at least 14, at least 15, at least 16, at least 17, at least 18, at least 19, at least 20, or at least 21 consecutive nucleic acids of any one of the nucleic acid sequences in Table 3B.

46. The method of claim 43, wherein the siRNA is at least 80%, at least 85%, at least 90%, or at least 95% identical to any one of the nucleic acid sequences in Table 3B.

47. The method of any one of claims 21-40, wherein the agent comprises an antisense oligonucleotide (ASO).

48. The method of any of claims 21-47, wherein the subject is a human subject.

49. The method of any one of claims 21-48, wherein the agent is administered intravenously or subcutaneously.

50. A composition comprising an siRNA comprising a nucleotide sequence selected from SEQ ID NOs: 4310-9964 or 9975-15629.
